(19) United States
(12) Patent Application Publication
(10) Pub. No.: US 2023/0243002 A1
(43) Pub. Date: Aug. 3, 2023

(54) ASSAY OT DETECT VIRUS

(71) Applicant: CORNELL UNIVERSITY, Ithaca, NY (US)

(72) Inventors: Roy COHEN, Ithaca, NY (US); Alexander TRAVIS, Ithaca, NY (US)

(21) Appl. No.: 17/917,058

(22) PCT Filed: Apr. 6, 2021
(86) PCT No.: PCT/US2021/025979

§ 371 Date: Oct. 5, 2022

Related U.S. Application Data
(60) Provisional application No. 63/005,785, filed on Apr. 6, 2020.

(51) Int. Cl. C12Q 1/70; C12Q 1/6816

(52) U.S. Cl. CPC C12Q 1/701; C12Q 1/6816

(57) ABSTRACT

The present invention relates to a method of detecting a target at least partially single-stranded viral genome in a sample. Also disclosed are kits and reaction mixtures.

This application claims the priority benefit of U.S. Provisional Patent Application Ser. No. 63/005,785, filed Apr. 6, 2021, which is hereby incorporated by reference in its entirety.

### FIELD

The present application relates to the use of enzymes to detect viruses.

### BACKGROUND

The detection and/or quantitation of specific nucleic acid sequences is an important technique for identifying and classifying microorganisms, diagnosing infectious diseases, detecting and characterizing genetic abnormalities, identifying genetic changes associated with cancer, studying genetic susceptibility to disease, and measuring response to various types of treatment. Such procedures are also useful in detecting and quantitating microorganisms in foodstuffs, water, industrial and environmental samples, seed stocks, and other types of material where the presence of specific microorganisms may need to be monitored.

Advances in the field of molecular biology over the last 20 years have allowed the detection of specific nucleic acid sequences in test samples obtained from patients and other subjects. Such test samples include serum, urine, stool, saliva, amniotic fluid, and other body fluids. Thus, a number of methods to detect and/or quantitate nucleic acid sequences are well known in the art. However, an inherent result of highly sensitive nucleic acid amplification systems is the emergence of side-products. Side-products include molecules which may, in some systems, interfere with the amplification reaction, thereby lowering specificity. This is because limited amplification resources, including primers and enzymes needed in the formation of primer extension and transcription products are diverted to the formation of side-products. In some situations, the appearance of side-products can also complicate the analysis of amplicon production by various molecular techniques. In addition, in many cases of interest, specific nucleic acid sequences are present at very low concentrations in the sample being tested for the required nucleic acid sequence. In such cases, if the assay sensitivity cannot be increased, the presence of the required molecule cannot be detected.

The emergence of severe acute respiratory coronavirus 2 (SARS-CoV-2), which causes coronavirus disease 2019 (COVID-19) in humans, has highlighted the great need for a sensitive and specific assay for the detection of viruses.

The present application is directed to overcoming these and other deficiencies in the art.

### SUMMARY

One aspect of the disclosure is a method of detecting a target at least partially single-stranded viral genome in a sample. The method involves providing a sample containing a target at least partially single-stranded viral genome; providing an oligonucleotide molecule complementary to a selected portion of the target at least partially single-stranded viral genome adjacent to a single-stranded template region of the target at least partially single-stranded viral genome; contacting the sample with the oligonucleotide molecule so that the oligonucleotide molecule hybridizes to the selected portion of the target at least partially single-stranded viral genome and forms a hybridization product comprising a double-stranded nucleic acid start portion in the viral genome at a location corresponding to the selected portion and the single-stranded template region is adjacent to the double-stranded nucleic acid start portion; contacting the hybridization product with a polymerase and a dNTP mixture to form a polymerase extension mixture; subjecting the polymerase extension mixture to conditions under which the hybridization product from the double-stranded nucleic acid start portion is extended by addition of nucleotides complementary to the single-stranded template region, thereby releasing free phosphates; producing adenosine triphosphates from the released free phosphates; and metabolizing the adenosine triphosphates produced from the free phosphates to produce a readout signal, indicating the presence of the target at least partially single-stranded viral genome in the sample.

Another aspect of the present disclosure is a kit for detecting a target at least partially single-stranded viral genome in a sample. The kit includes: a polymerase; a dNTP mixture; one or more enzymes for producing adenosine triphosphates from released free phosphates; and a luciferase for producing a bioluminescent readout signal. In some embodiments, the kit further includes one or more oligonucleotide molecules complementary to a selected portion of a target at least partially single-stranded viral genome adjacent to a single-stranded template region of the target at least partially single-stranded viral genome.

Another aspect of the disclosure is a method of treating a human. The method involves: obtaining a sample from a human; determining a presence or an absence of at least a portion of a single-stranded viral genome in the sample by one or more of the methods of detecting a target described herein; and treating the human based on the presence or absence of at least a portion of the single-stranded genome in the sample.

Yet another aspect of the disclosure is a method of treating a non-human subject. The method involves: obtaining a sample from a non-human subject; determining a presence or an absence of at least a portion of a single-stranded viral genome in the sample by one or more of the methods of detecting a target described herein; and treating the non-human subject based on the presence or absence of at least a portion of the single-stranded genome in the sample.

### BRIEF DESCRIPTION OF THE DRAWINGS

FIGS. 1A-1B show a schematic of an exemplary embodiment of an assay according to methods described herein and a graph showing the theoretical range for PPi release per 1× copy of viral genome. FIG. 1A shows steps 1-9 of the exemplary assay according to methods described herein. Step 1 is the ID-Oligo design process, which in some embodiments involves (i) identifying uracil/thymine-free regions in the target at least partially single-stranded viral genome (ii) identifying virus-specific regions immediately 3′ or 5′ to those uracil/thymine-free regions (depending on whether the target virus has a positive or negative sense genome), where the reverse complement sequences of the virus-specific regions will be the “ID-Oligos”; (iii) selecting, based on (i) and (ii), one or more sequences for use as ID-Oligos that will hybridize with the genomic RNA/DNA (in some embodiments, ID-Oligos are between 6-25 nucleotides long); (iv) determining the number and specificity of ID-Oligos based on the detection goals, and nature of viral targets; (v) analyzing the one or more ID-Oligos for self-dimerization and cross-dimerization; and (iv) analyzing genomic ssRNA/ssDNA for areas of secondary structure that might inhibit ID-Oligo hybridization. Step 2 involves obtaining a sample. Step 3 involves the annealing of full or partial ssRNA/ssDNA (e.g., a viral genome from the sample or sample extract) with one or more ID-Oligos (each pre-designed to have a complementary sequence to the target domain within the viral genome as described herein). Step 4 involves the binding of reverse-transcriptase (RT) and/or DNA-Polymerase to the newly formed double strand RNA/DNA hybrid or dsDNA. Step 5 involves the extension of the complementary RNA/DNA strand from the hybridized ID-Oligos. Step 6 involves the release of phosphate groups (PPi, 2 per nucleotide added to the ssRNA/ssDNA strand) by the extension/polymerization reaction. Step 7 involves the conversion of released phosphates into ATP by downstream enzymatic reactions (e.g., in some embodiments, the enzymes are tethered enzymes). In some embodiments, free phosphates and APS (adenosine 5′-phosphosulfate) are converted into ATP by ATP-sulfurylase. In other embodiments, free phosphates along with NAD/GAP/ADP are converted into ATP by the coupled reaction of GAPDH-PGK. Step 8 involves metabolizing ATP by luciferase (e.g., soluble luciferase or luciferase tethered to nanoparticles) to produce bioluminescence as the readout signal. Step 9 involves quantifying the readout luminescence signal to determine the presence/relative abundance or concentration of the target viral genome. FIG. 1B shows the theoretical range for PPi release per 1× copy of the viral genome, as a function of the uracil/thymine-free region's length and the number of oligonucleotide molecules complementary to a selected portion of a target at least partially single-stranded viral genome (i.e., ID-Oligo or “primer”) included with a the reaction mixture.

FIG. 2 illustrates part of the process for the design of oligonucleotide molecules complementary to a selected portion of a target at least partially single-stranded viral genome (i.e., ID-Oligo or “primer”) shown in Step 2 of FIG. 1A. In the illustrated embodiment, ID-Oligo molecules were designed to bind adjacent to a uracil/thymine-free region in the target at least partially single-stranded viral genome (Table 11). FIG. 2 shows an alignment of sequences from Table 11 against genomes from 7 other coronaviruses. Colored regions (red or darker shaded) indicate misalignment between the sequences. The top panel in FIG. 2 shows the first portion of the sequence alignment. The bottom panel in FIG. 2 shows the second portion of the sequence alignment, with some overlap with the top panel.

FIG. 3 shows the results of analysis of ID-Oligos designed for the human coronavirus 229E using analytical software. FIG. 3 identifies ID-Oligos that self-dimerize and cross-dimerize with each other.

FIG. 4 is a schematic diagram illustrating a method of detecting a target at least partially single-stranded viral genome (“target viral genome”). The method involves providing an ID-Oligo (a “primer” or oligonucleotide molecule complementary to a selected portion of a target at least partially single-stranded viral genome) adjacent to a single-stranded region (“single-stranded template region”) of the target viral genome; contacting the ID-Oligo with the target viral genome so that the ID-Oligo hybridizes to the selected portion of the target viral genome and forms a hybridization product comprising a double-stranded nucleic acid start portion in the viral genome at a location corresponding to the selected portion and the single-stranded template region is adjacent to the double-stranded nucleic acid start portion; contacting the hybridization product with a polymerase (e.g., RT (shown) or DNA polymerase) and a dNTP mixture (comprising, e.g., dGTP, dCTP, dTTP, dATP, and/or dATPαS) to form a polymerase extension mixture; subjecting the polymerase extension mixture to conditions under which the hybridization product from the double-stranded nucleic acid start portion is extended by addition of nucleotides complementary to the single-stranded template region, thereby releasing free phosphates; and producing adenosine triphosphates from the released free phosphates (PPi). In the presence of adenosine 5′-phosphosulfate, the PPi groups are used by the enzyme ATP-sulfurylase to generate adenosine triphosphate (ATP). Luciferase hydrolyses the ATP to generate a bioluminescent signal (a readout signal), which indicates the presence of the target viral genome. NP: nanoparticles; ssDNA: single-strand deoxyribonucleic acid; ssRNA: single-strand ribonucleic acid; RT: reverse transcriptase; PPi/Pi: free phosphates; ATP-sul: ATP sulfurylase; ATP: adenosine triphosphate; APS: adenosine 5′-phosphosulfate; SO⁴⁺: sulfate; Lun: luciferin; Mg²⁺: magnesium; Luc: luciferase; and AMP: adenosine monophosphate.

FIG. 5 is a schematic diagram illustrating a method of detecting a target at least partially single-stranded viral genome (“target viral genome”). The method involves providing an ID-Oligo (a “primer” or oligonucleotide molecule complementary to a selected portion of a target at least partially single-stranded viral genome) adjacent to a single-stranded region (“single-stranded template region”) of the target viral genome; contacting the ID-Oligo with the target viral genome so that the ID-Oligo hybridizes to the selected portion of the target viral genome and forms a hybridization product comprising a double-stranded nucleic acid start portion in the viral genome at a location corresponding to the selected portion and the single-stranded template region is adjacent to the double-stranded nucleic acid start portion; contacting the hybridization product with a polymerase (e.g., RT (shown) or DNA polymerase) and a dNTP mixture (comprising, e.g., dGTP, dCTP, dTTP, and optionally dATP, and/or dATPαS) to form a polymerase extension mixture; subjecting the polymerase extension mixture to conditions under which the hybridization product from the double-stranded nucleic acid start portion is extended by addition of nucleotides complementary to the single-stranded template region, thereby releasing free phosphates; and producing adenosine triphosphates from the released free phosphates (PPi). In the presence of ADP, NAD⁺, and GAP, these PPi groups are used by the coupled GAPDH-PGK enzymatic reaction to generate ATP. Luciferase hydrolyses the ATP to generate a bioluminescence signal (a readout signal), which indicates the presence of the target viral genome. NP: nanoparticles; ssDNA: single-strand deoxyribonucleic acid; ssRNA: single-strand ribonucleic acid; RT: reverse transcriptase; PPi/Pi: free phosphates; NAD⁺/NADH: nicotinamide adenine dinucleotide; GAP: glyceraldehyde 3-phosphate; GAPDH: glyceraldehyde 3-phosphate dehydrogenase; PGK: phosphoglycerate kinase; ATP: adenosine triphosphate; 3PG: 3-phosphoglyceric acid; Lun: luciferin; Mg: magnesium; Luc: luciferase; and AMP: adenosine monophosphate.

FIGS. 6A-6C demonstrate the use of ID-Oligos (i.e., oligonucleotide molecules complementary to a selected portion of a target at least partially single-stranded viral genome (“primer”)) to detect 3 synthetic fragments of the SARS-CoV-Urbani genome using the method described in FIG. 4. FIG. 6A shows the sequence of the three synthetic ssDNA partial sequences of the SARS coronavirus Urbani virus genome (IDT, Iowa USA)

(SEQ ID NO: 276)
(SARS-C-U#1:
(5′)CTAAACAAGAGGAGCCACCAAACACAGAAGATTCCAAAACTGAGGA
GAAATCTGTCGTACA (3′);
(SEQ ID NO: 277)
SARS-C-U#2:
(5′)GGTAAAGGCCAACAACAACAAGGCCAAACTGTCACTAAGAAATCTG
CTGCTGAGG(3′);
(SEQ ID NO: 278)
SARS-C-U#3:
(5′)CTAAAAAGGACAAAAAGAAAAAGACTGATGAAGCTCAGCCTTTGCC
GCAGAG(3′)).

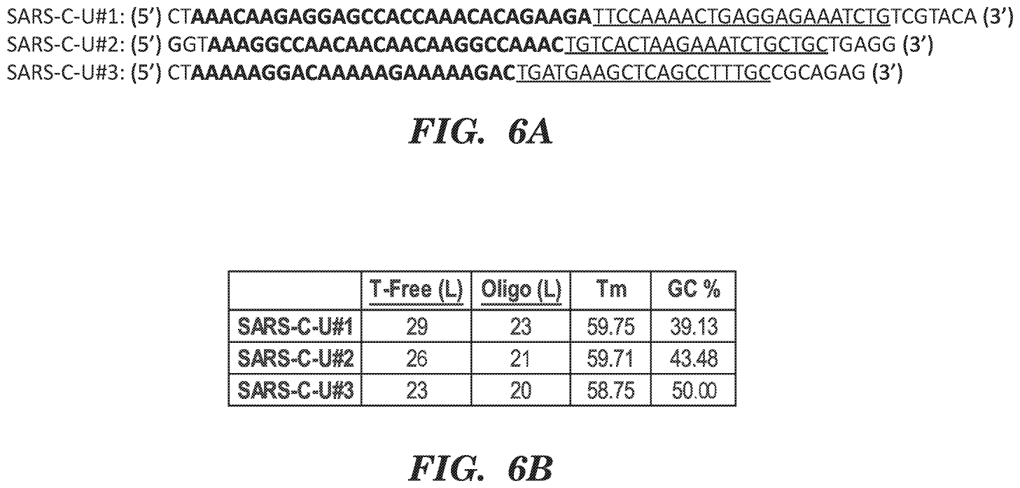

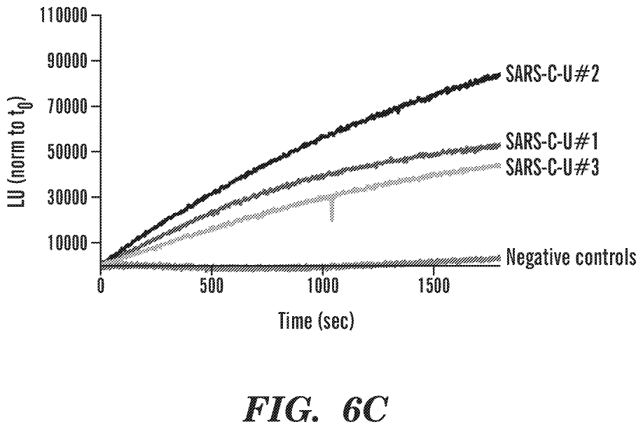

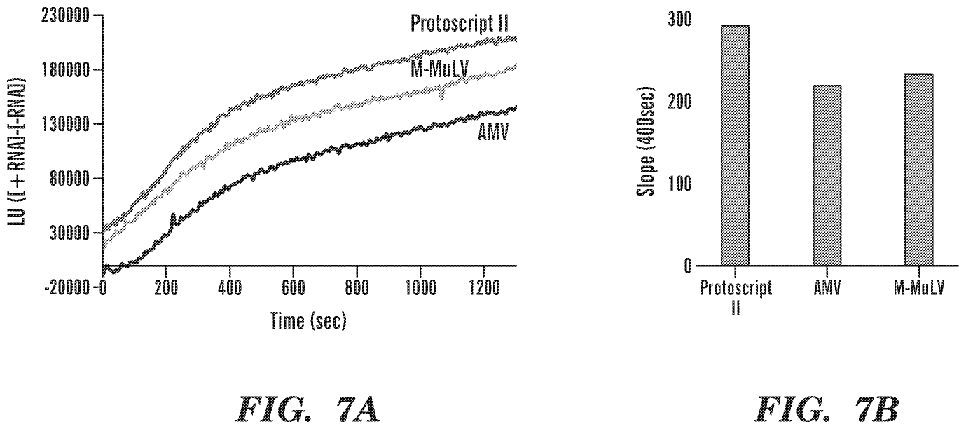

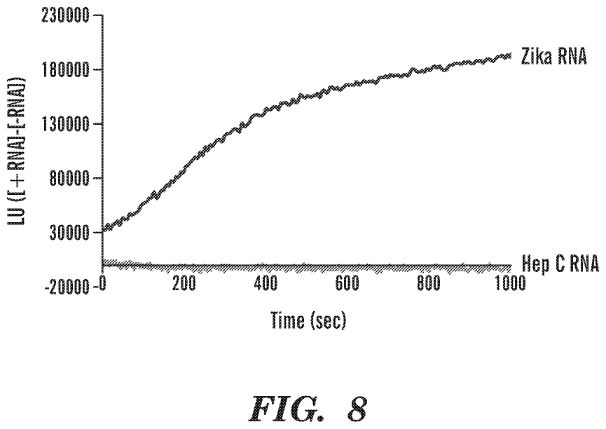

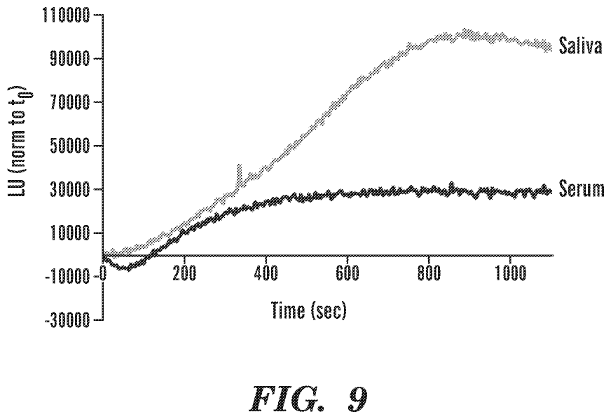

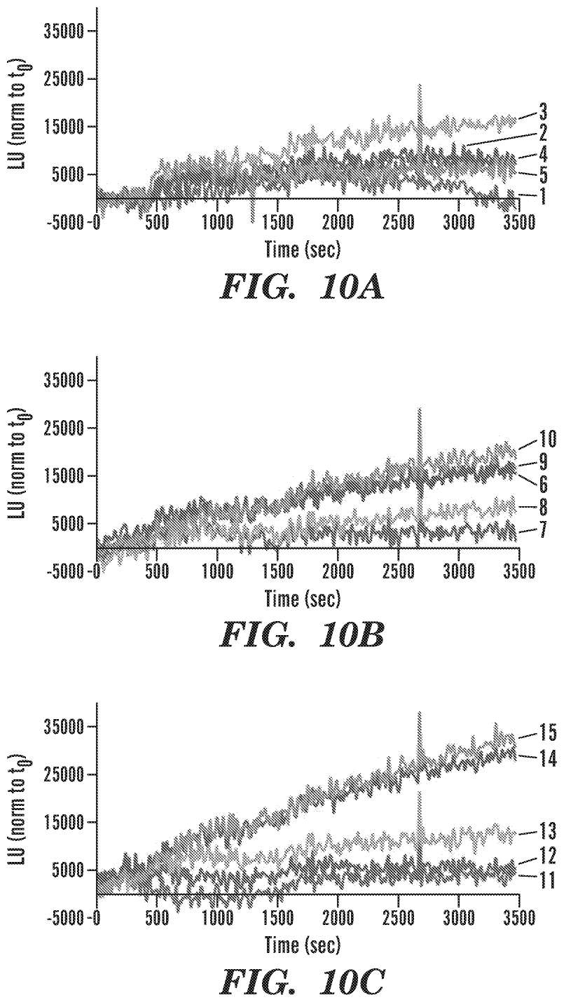

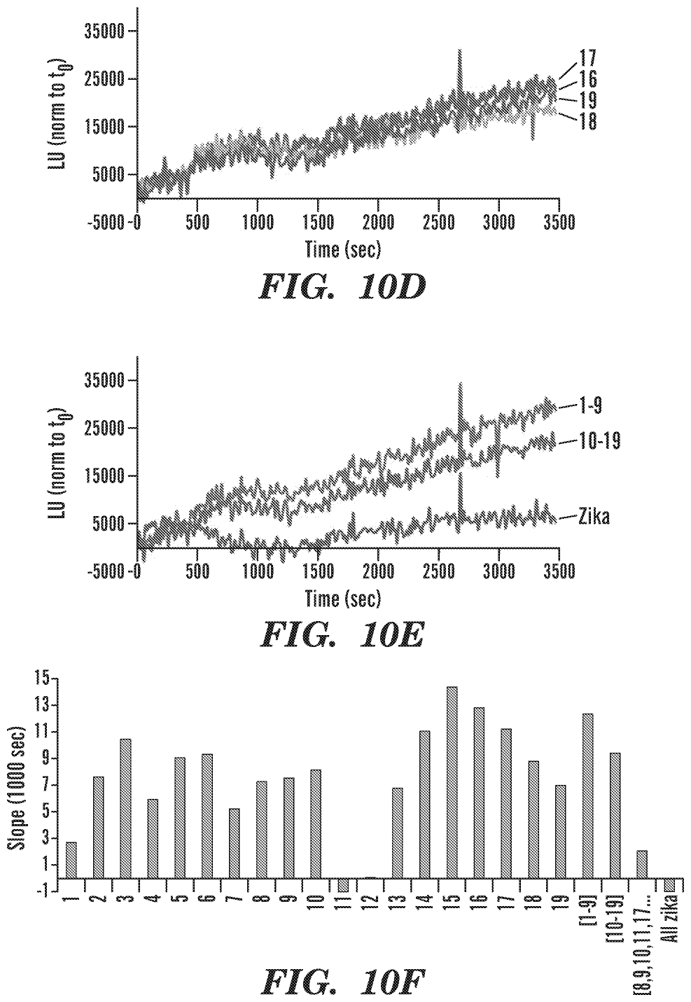

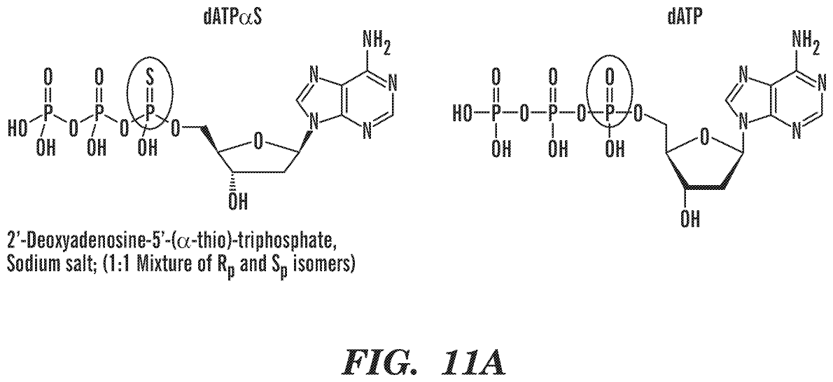

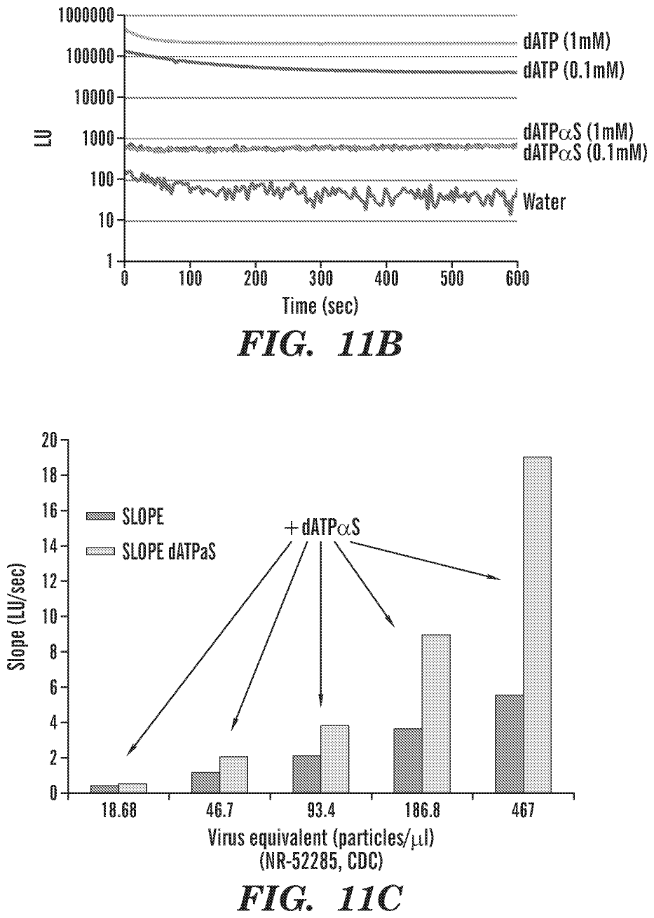

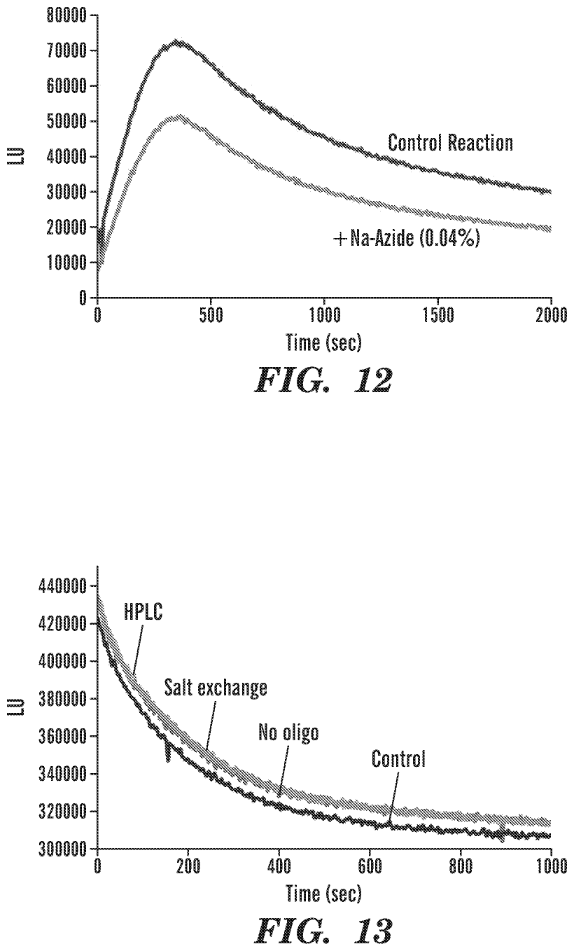

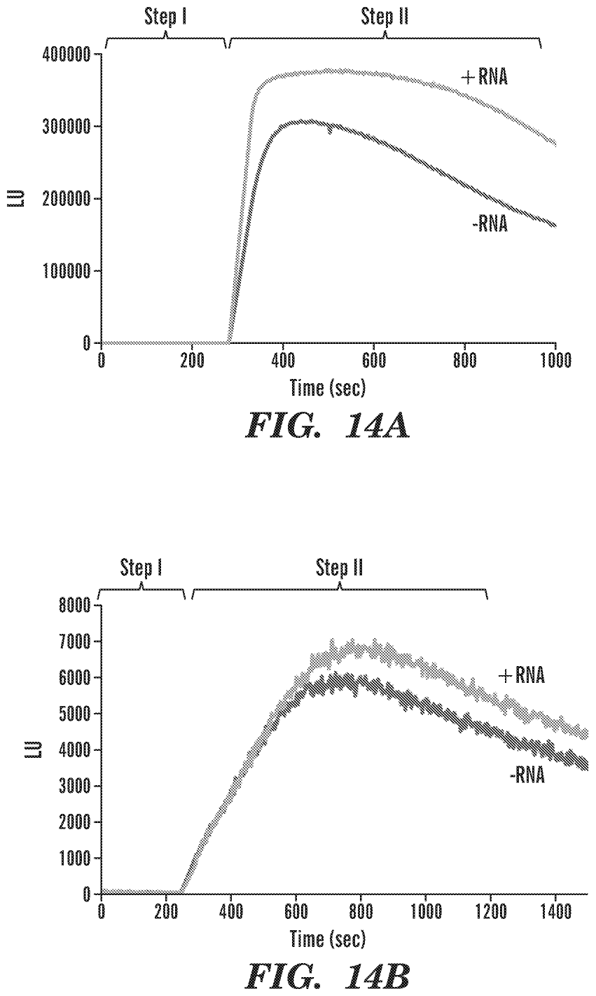

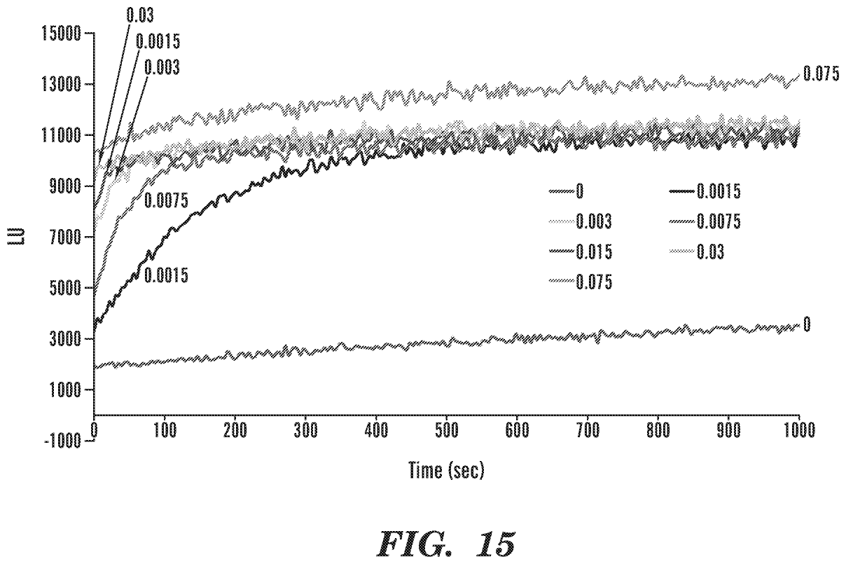

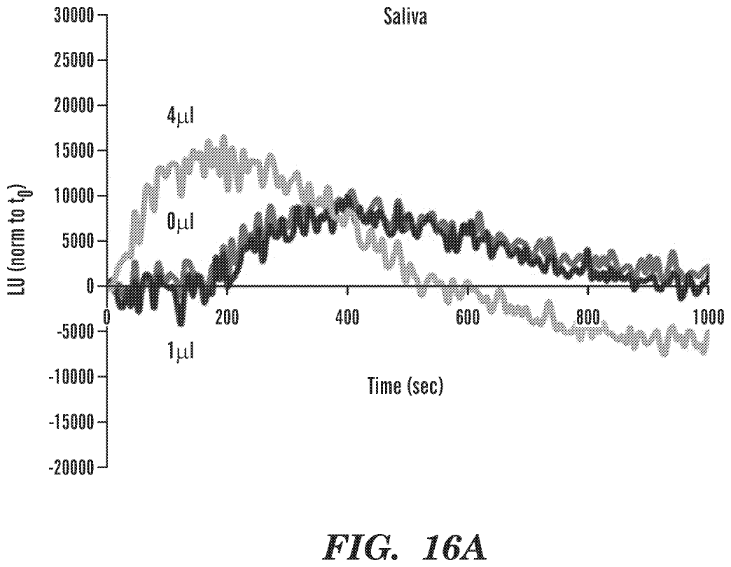

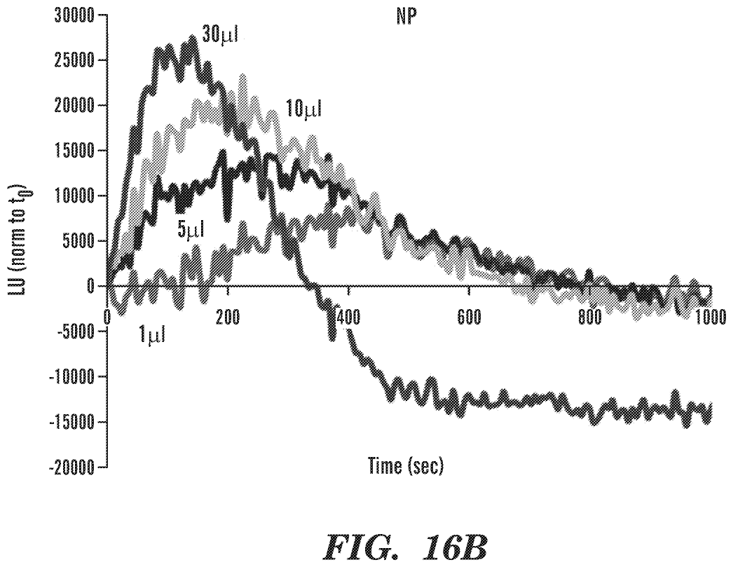

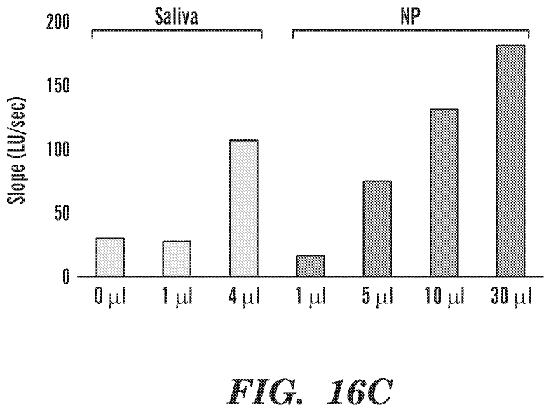

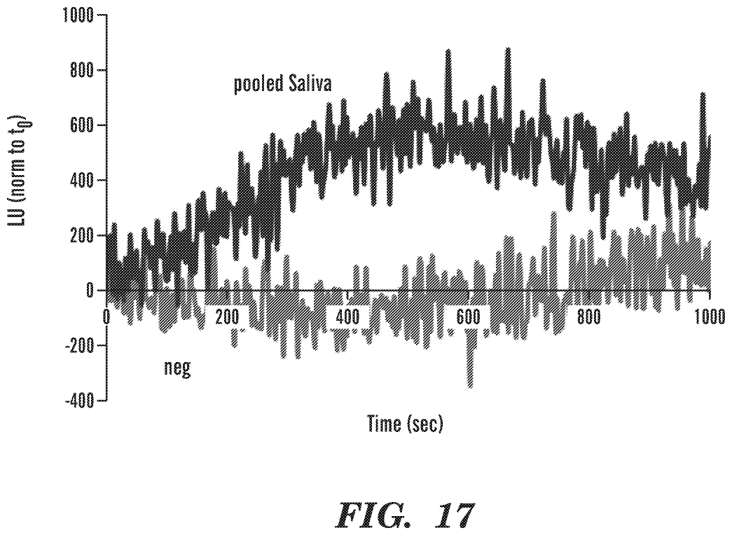

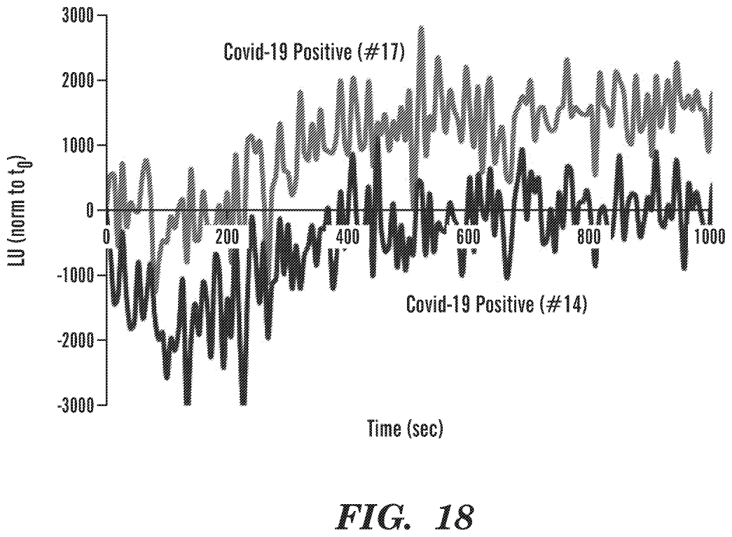

Each synthetic sequence includes a thymine-free region (bold) and a target sequence for the ID-Oligo to bind (underline). FIG. 6B is a table showing the main properties of the synthetic genome fragments shown in FIG. 6A, including the length in nucleotides of the thymine-free region (T-Free (L)), the length in nucleotides of the ID-Oligo (Oligo (L)), the melting temperature of the ID-Oligo (Tm, provided in Celsius), and the GC content of the ID-Oligos (GC %). FIG. 6C is a graph showing the luminescent signal generated by the reaction according to the methods of the disclosure in the presence of each of the 3 synthetic genome fragments and negative controls (reactions lacking the synthetic genome fragments). The master mix included: His-Si-Luc (0.8 μl), ATPS (NEB, 0.8 μl), BST (DNA Polymerase, 0.8 NEB), buffer (HEPES, MgCl₂, NaCl, pH7.4), luciferin (1 mM), APS (0.06 mM), dNTP mix (0.05 mM of each dTTP, dCTP, dGTP). The master mix was added to individual wells in a 96-well microplate containing ID-Oligos, as indicated in FIG. 6A, and a SARS-CoV-Urbani sequence or water. Luminescent signal was integrated every 0.4 seconds and measured for up to 50 minutes at room temperature.

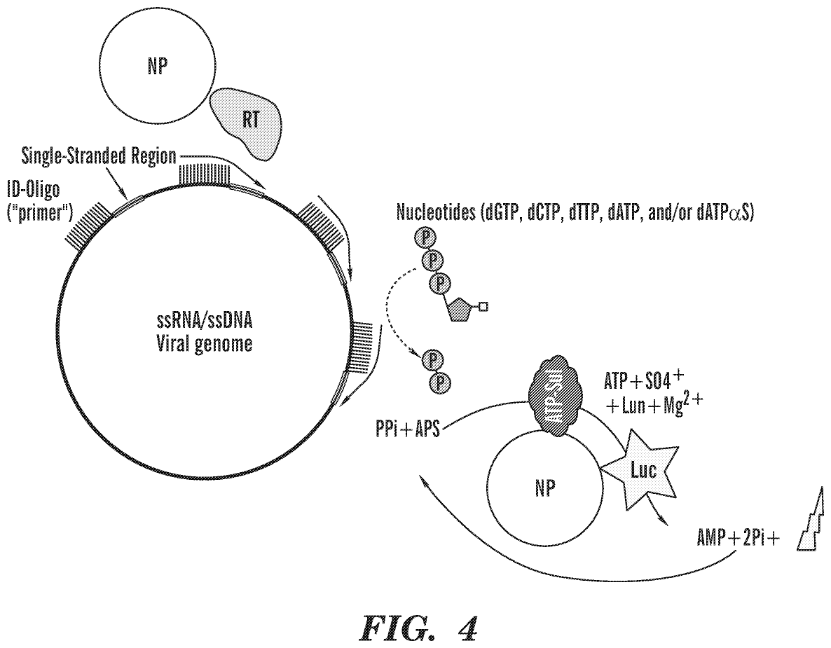

FIGS. 7A-7B shows a comparison of three different reverse transcriptase (RT) enzymes when using the methods described in FIG. 4 to detect RNA isolated from Zika virus. FIG. 7A is a graph showing the results of three reactions, each including one of the following RTs: ProtoScript® II RT, M-MuLV RT, and AMV RT (New England Biolabs, MA, USA). The master mix used for each reaction included His-Si-Luc (0.8 μl), ATPS (NEB, 0.8 μl), buffer (HEPES, MgCl₂, NaCl, pH7.4), luciferin (1 mM), APS (0.06 mM), and dNTP mix (0.0 5 mM of each dTTP, dCTP, dGTP). Master mix was added to individual wells in a 96-well microplate containing RTs as indicated in FIG. 7A (2 all from NEB), and RNA or water. Luminescent signal was integrated every 0.4 seconds and measured for up to 50 minutes at room temperature. FIG. 7B is a graph showing the assay kinetics as measured from the reaction initial slope (calculated for the linear region during 0-400 seconds). Slope (LU/time) is one way to analyze the reaction signal. Other approaches include, for example, calculating the integrated signal over a defined time frame, maximal peak signal, peak signal normalized to the baseline LU, peak to slope ratio, and various combinations two or more of these approaches.

FIG. 8 demonstrates the ability of the ID-Oligos to distinguish between RNA from different viral species. A set of ID-Oligos specific for regions within the Zika virus genome were mixed with the assay reagents, which included His-Si-Luc (20 μl), ATPS (NEB, 1 μl), buffer (HEPES, MgCl₂, NaCl, pH7.4), luciferin (10 mM), APS (0.06 mM), dNTP mix (0.05 mM of each dTTP, dCTP, dGTP). The master mix was then added to individual wells in a 96-well microplate containing RNA from Zika virus (strain KX26887.1), Hepatitis C virus (strain JFH-1), or water, and primers designed to identify the Zika RNA. Luminescent signal was integrated every 0.4 seconds, and measured for up to 50 minutes at room temperature. The data in FIG. 8 show that using Zika-specific ID-Oligos did not result in cross-reactivity with RNA from Hepatitis C virus. Both Zika and Hepatitis C are RNA viruses in the same viral family (Flaviviridae).

FIG. 9 is a graph showing experimental results which demonstrate that ID-Oligos can be used to detect a target single-stranded viral genome in a sample comprising human serum and human saliva. Assay reagent mixtures comprising His-Si-Luc (20 μl), ATPS (NEB, 1 μl), Klenow (1 μl, NEB), buffer (HEPES, MgCl₂, NaCl, pH7.4), luciferin (10 mM), APS (0.06 mM), dNTP mix (0.05 mM of each dTTP, dCTP, dGTP), and oligos (1 μm of each of 3 oligos) were prepared as a master mix. The master mix was added to individual wells in a 96-well microplate containing ssDNA diluted in saliva or serum, and luminescence signal was measured for up to 50 minutes (at room temperature).

FIGS. 10A-10F demonstrate the reaction kinetics for detection of RNA isolated from the common human coronavirus 229E in the presence of various ID-Oligos. In brief, 19 different ID-Oligos (corresponding to SEQ ID NOs:251-269) were added to the reaction mixture in the presence of 0.1 pg RNA isolated from human coronavirus 229E (ATCC). FIGS. 10A-10D show the reaction kinetics in the presence of individual ID-Oligos 1-5 (corresponding to SEQ ID NOs:251-255, respectively) (FIG. 10A), ID-Oligos 6-10 (corresponding to SEQ ID NOs:256-260, respectively) (FIG. 10B), ID-Oligos 11-15 (corresponding to SEQ ID NOs:261-265, respectively) (FIG. 10C), ID-Oligos 17-19 (corresponding to SEQ ID NOs:267-269, respectively) (FIG. 10D). FIG. 10E shows the reaction kinetics in the presence of mixtures of ID-Oligos 1-9 (corresponding to SEQ ID NOs:251-259, respectively) and ID-Oligos 10-19 (corresponding to SEQ ID NOs:260-269, respectively), or in the presence of a set of ID-Oligos designed to detect the Zika virus. FIG. 10F is a summary of the reaction kinetics (slope) in the presence of each ID-Oligo and combinations. The mixture included: His-Si-Luc (20 μl), ATPS (NEB, 1 μl), ProtoScript® II RT (1 μl, NEB), buffer (HEPES, MgCl₂, NaCl, pH7.4), luciferin (10 mM), APS (0.06 mM), dNTP mix (0.05 mM of each dTTP, dCTP, dGTP), and ID-Oligos (as indicated, 1 micromolar of each). Master mix was added to individual wells in a 96-well microplate containing RNA or water (as indicated) and luminescence signal was measured for up to 50 minutes (at room temperature).

FIGS. 11A-11C demonstrate the use of dATPαS to enable inclusion of ID-Oligos targeting non-uracil/thymine-free regions of a target single-stranded viral genome isolated from a SARS-CoV-2, USA-WA1/2020 oropharyngeal swab (BEI Cat #NR-52285). The sequence of this genome corresponds to GenBank Accession No. MN985325, which is hereby incorporated by reference in its entirety (SEQ ID NO:279, see Table 9). FIG. 11A shows the structural differences between dATP and dATPαS. FIG. 11B shows data from a reaction including only the luciferase and no viral RNA. Specifically, FIG. 11B is a graph showing that replacing dATP with dATPαS reduces the background LU in the ID-Oligos assay reaction, if one wishes to use ID-Oligos that are not adjacent to uracil/thymine-free regions. Master mix for 5 reactions included: His-Si-Luc (5 μl), buffer (HEPES, MgCl₂, NaCl, pH7.4), luciferin (10 mM), APS, dNTP mix (0.06 mM of each dTTP, dCTP, dGTP) and either 0.1 mM/1 mM of dATP, 0.1 mM/1 mM of dATPαS or water. The plot shows the reduction in the LU background when using dATPαS versus dATP, which provides a significantly improved signal-to-noise ratio (— 2 orders of magnitude) and thus higher overall sensitivity of the assay. FIG. 11C is a graph showing the comparison of ID-Oligos reaction signal output with only dC/T/G or with dC/T/C+dATPαS. A master mix for reactions included: His-Si-Luc (5 μl), ATPS (NEB, 0.2 μl), ProtoScript® II RT (NEB, 0.2 μl), buffer (HEPES, MgCl₂, NaCl, pH7.4), luciferin (10 mM), APS (0.06 mM), dNTP mix (0.05 mM of each dTTP, dCTP, dGTP), +/−0.1 mM dATPαS, and oligo mix (ID-Oligos corresponding to SEQ ID NOs:30-48 of Table 11 (i.e., ID-Oligos having the sequence of SEQ ID NOs:110-128 of Table 11)+ID-Oligos corresponding to SEQ ID NOs:69-89 of Table 11 (i.e., ID-Oligos having sequence of SEQ ID NOs:149-169 of Table 11)). Blue bars (left bar in each set)—reaction slopes when only dT/G/C's are included. Red bars (right bar in each set)—reaction slopes when supplemented with dATPαS. Higher slope values indicate more dynamic assay kinetics due to the longer polymerization reaction step by the RT.

FIG. 12 shows the inhibitory effect of Na-Azide on the luminescence output of the coupled ATPS-luciferase reaction. Master mix was prepared for 2 reactions by combining His-Si-Luc (5 μl), His-Si-ATPS (5 μl), buffer (HEPES, MgCl₂, NaCl, pH7.4), luciferin (10 mM), APS (0.33 mM), and NaPPi (2.2 mM). One well was supplemented with 0.04% Na-Azide, and LU was measured for 200 seconds.

FIG. 13 demonstrates that different methods of ID-Oligo preparation/purification do not add to background LU. To check if ID-Oligos add any background LU due to carry over of PPi produced during their synthesis process, ID-Oligo purification via HPLC and standard desalting (salt exchange) were evaluated in comparison to non-purified ID-Oligos (control). Master mix included: His-Si-Luc (10 ul), buffer (HEPES, MgCl₂, NaCl, pH7.4), luciferin (10 mM), ATPS (1 μl, NEB), and APS (0.33 mM). 97 μl of master mix was added to wells (96-well microplate) containing 3 μl of 100 μM of ID-Oligos, or water (no oligo-control). LU was measured for up to 50 minutes.

FIGS. 14A-14B demonstrate the detection of synthetic SARS-CoV-2 using an exemplary 2-step assay according to methods described herein. In this version of the assay, the reverse transcriptase (RT) (Mu-MLV) was mixed with ID-Oligos, reagents, and RNA for an initial hybridization/polymerization step (Step I). Then, the luciferase (Luc) and ATPS were added to transduce PPi formation (by the RT) into ATP and luminescence (Step II). Master mix comprising His-Si-Mu-MLV (7 μl), buffer (HEPES, MgCl₂, NaCl, pH7.4), luciferin (10 mM), oligo (CV3-oligo, 1 micromolar), APS (0.33 mM), and RNA (synthetic SARS-CoV-2 (CV3, 0.3 micromolar) or water (“-RNA” control reaction) was prepared for two reactions. 88 μl of master mix was added to wells (96-well microplate), and luminescence (LU) was measured for 250-300 seconds (Step I). At 250/300 second, a mix of His-Si-Luc (5 μl) and His-Si-ATPS (6 μl) was added to both wells to initiate step II of the reaction. FIG. 14A shows the results of the ID-Oligos assay using enzymes in solution. FIG. 14B shows the results of the ID-Oligos assay using enzymes attached to NPs (500 nm, SiO₂). The data (i) confirm that luminescence is not detected until addition of luciferase and ATPS and (ii) demonstrate that the Oligo-ID assay may be carried out using enzymes in solution or enzymes attached to NPs.

FIG. 15 is a graph demonstrating that different ATPS/RT ratios affect reaction kinetics. A reaction master mix for 7 reactions was prepared comprising His-Si-Luc (10 μl), buffer (HEPES, MgCl₂, NaCl, pH7.4), luciferin (10 mM), APS (0.33 mM), dNTP mix (0.05 mM of each dTTP, dCTP, dGTP, and dATPαS), 40× oligo mix, SARS-CoV-2 RNA (#N-52285, 1:500 dilution), Mu-MLV RT (0.2 ul NEB[200 U/μl]), and increasing volumes of ATPS (NEB [3 U/μl]). Master mix was added to wells (96-well microplate) containing ATPS or water (no oligo) to result in the following unit ratios of ATPS/RT: 0, 0.0015, 0.003, 0.0075, 0.015, 0.03, 0.075. LU was measured for up to 50 minutes.

FIGS. 16A-16C show the detection of SARS-CoV-2 RNA in saliva or nasopharyngeal samples from patients diagnosed with COVID-19. Paired saliva (S) and nasopharyngeal (NP) samples were collected from patients known to have confirmed COVID-19 infection (Guthrie Medical Center, Sayre, Pa.). FIG. 16A and FIG. 16B are graphs showing the change in luminescence (LU) over time in the saliva sample (FIG. 16A) and nasopharyngeal sample (FIG. 16B), respectively. FIG. 16C is a bar plot showing the rate of luminescence output (slope) as a function of RNA quantity in individual wells.

FIG. 17 is graph showing the detection of SARS-CoV-2 RNA in pooled saliva from patients diagnosed with COVID-19. RNA extracted from saliva (S) of 4 donors were pooled into a single sample (collected from patients known to have confirmed COVID-19 infection, Guthrie Medical Center, Sayer Pa.). FIG. 17 shows the change in luminescence units (LU, normalized to time point 0) over time.

FIG. 18 is a graph showing the detection of SARS-CoV-2 RNA in nasopharyngeal (NP) samples from COVID-19 positive or negative donors. RNA extracted from NP samples collected from 1 confirmed Covid-19 positive donor (#17) and 1 confirmed negative donor (#14) Guthrie Medical Center, Sayre, Pa.). FIG. 18 shows the change in luminescence units (LU, normalized to time point 0) over time.

### DETAILED DESCRIPTION

Unless otherwise indicated, the definitions and embodiments described in this and other sections are intended to be applicable to all embodiments and aspects of the present application herein described for which they are suitable as would be understood by a person skilled in the art.

As used herein, the singular forms “a,” “an,” and “the” and the like include plural referents unless the context clearly dictates otherwise. Thus, for example, reference to “a compound” includes both a single compound and a plurality of different compounds.

Terms of degree such as “substantially”, “about”, and “approximately” as used herein mean a reasonable amount of deviation of the modified term such that the end result is not significantly changed. These terms of degree should be construed as including a deviation of at least ±1% (and up to ±5% or ±10%) of the modified term if this deviation would not negate the meaning of the word it modifies.

The term “and/or” as used herein means that the listed items are present, or used, individually or in combination. In effect, this term means that “at least one of” or “one or more” of the listed items is used or present.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, and so on. As a non-limiting example, each range discussed herein can be readily broken down into a lower third, middle third and upper third, and so on. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “involving”, “having”, and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. In embodiments or claims where the term comprising (or the like) is used as the transition phrase, such embodiments can also be envisioned with replacement of the term “comprising” with the terms “consisting of” or “consisting essentially of.” The methods, kits, systems, and/or compositions of the present disclosure can comprise, consist essentially of, or consist of, the components disclosed.

In embodiments comprising an “additional” or “second” component, the second component as used herein is different from the other components or first component. A “third” component is different from the other, first, and second components, and further enumerated or “additional” components are similarly different.

Certain terms employed in the specification, examples and claims are collected herein. Unless defined otherwise, all technical and scientific terms used in this disclosure have the same meanings as commonly understood by one of ordinary skill in the art to which this disclosure belongs.

Preferences and options for a given aspect, feature, embodiment, or parameter of the invention should, unless the context indicates otherwise, be regarded as having been disclosed in combination with any and all preferences and options for all other aspects, features, embodiments, and parameters of the invention.

Embodiments herein relate to assays useful in the detection of the presence of absence of viral genomic nucleic acids in a sample, as well as individual components of those assays, combinations of components of those assays, and related kits, devices, compositions, systems, and methods.

One aspect of the disclosure is a method of detecting a target at least partially single-stranded viral genome in a sample. The method involves providing a sample containing a target at least partially single-stranded viral genome; providing an oligonucleotide molecule complementary to a selected portion of the target at least partially single-stranded viral genome adjacent to a single-stranded template region of the target at least partially single-stranded viral genome; contacting the sample with the oligonucleotide molecule so that the oligonucleotide molecule hybridizes to the selected portion of the target at least partially single-stranded viral genome and forms a hybridization product comprising a double-stranded nucleic acid start portion in the viral genome at a location corresponding to the selected portion and the single-stranded template region is adjacent to the double-stranded nucleic acid start portion; contacting the hybridization product with a polymerase and a dNTP mixture to form a polymerase extension mixture; subjecting the polymerase extension mixture to conditions under which the hybridization product from the double-stranded nucleic acid start portion is extended by addition of nucleotides complementary to the single-stranded template region, thereby releasing free phosphates; producing adenosine triphosphates from the released free phosphates; and metabolizing the adenosine triphosphates produced from the free phosphates to produce a readout signal, indicating the presence of the target at least partially single-stranded viral genome in the sample. In some embodiments the single-stranded template region is a uracil/thymine-free region.

Also contemplated are methods of detecting the presence or absence of a target at least partially single-stranded viral genome (target viral genome) in a sample. In some embodiments, the method involves carrying out the steps of the methods described herein, wherein the target viral genome is present in the sample and a readout signal is produced. In some embodiments, the method involves carrying out the steps of the methods described herein, wherein the target viral genome is not present in (or is absent from) the sample and a readout signal is thereby not produced. For example, in some embodiments, the sample is contacted with an oligonucleotide molecule complementary to a selected portion of a target viral genome under conditions whereby the oligonucleotide molecule hybridizes to the selected portion of the target viral genome (if present in the sample) to form a hybridization product. The sample is contacted with a polymerase and a dNTP mixture and subjected to conditions under which the hybridization product (if present) is extended from a double-stranded nucleic acid start portion by addition of nucleotides complementary to a single-stranded template region, as described herein, (if present) thereby releasing free phosphates. Where the target viral genome is absent from the sample, adenosine triphosphates are not produced from free phosphates released as a result of a polymerization extension reaction, the adenosine triphosphates are thereby not metabolized, and a readout signal is not produced; absence of the readout signal indicates the absence of the target viral genome in the sample.

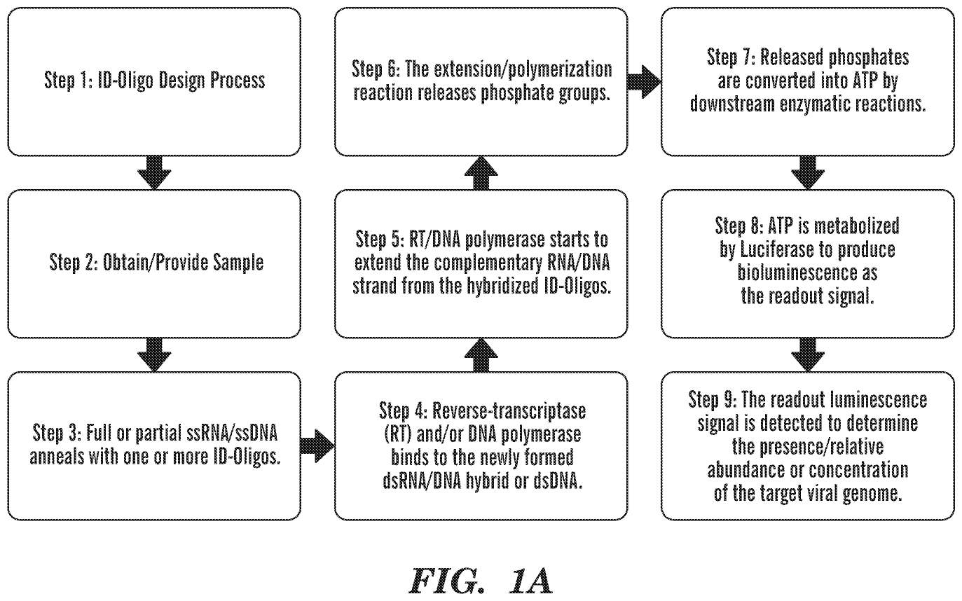

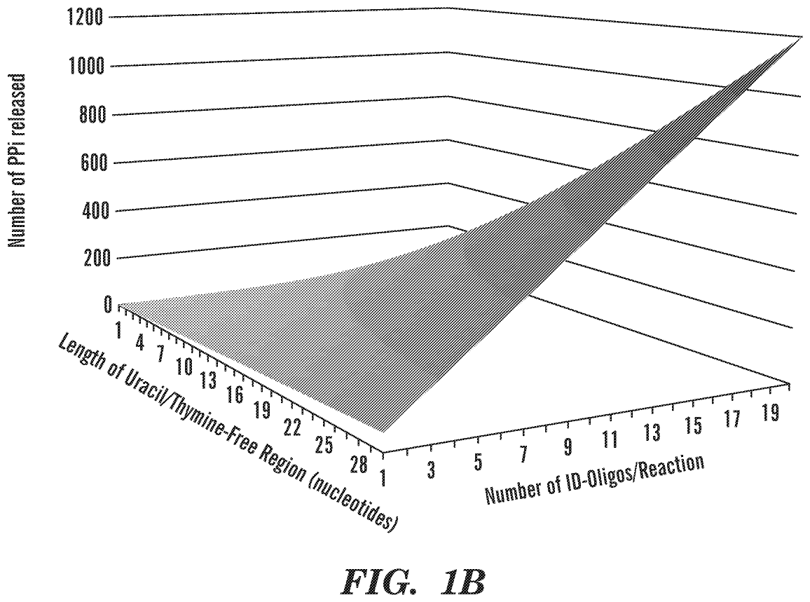

An exemplary embodiment is shown schematically in FIG. 1A. In this embodiment, there are nine steps. It will be understood that although shown and described in connection with this exemplary embodiment, each step (or various combinations thereof) may be used in accordance with any embodiment of the disclosure. As shown in FIG. 1A, step 1 an the ID-Oligo design process, which may involve (i) identifying uracil/thymine-free regions in the target at least partially single-stranded viral genome (ii) identifying virus-specific regions immediately 3′ or 5′ to those uracil/thymine-free regions (depending on whether the target virus has a positive or negative sense genome), where the reverse complement sequences of the virus-specific regions will be the “ID-Oligos”; (iii) selecting, based on (i) and (ii), one or more sequences for use as ID-Oligos that will hybridize with the genomic RNA/DNA in some embodiments, ID-Oligos are between 6-25 nucleotides long); (iv) determining the number and specificity of ID-Oligos based on the detection goals and the nature of the target at least partially single-stranded viral genome; (v) analyzing the one or more ID-Oligos for self-dimerization and cross-dimerization; and (iv) analyzing genomic ssRNA/ssDNA for areas of secondary structure that might inhibit ID-Oligo hybridization.

Depending on the objectives, and as described herein, ID-Oligos may be designed to hybridize with regions conserved within a family or genus of viruses (e.g., to identify any betacoronavirus, if that is the target) or to one or more regions specific to an individual species of virus (e.g., SARS-CoV), or to one or more regions specific to an individual strain of virus (e.g., SARS-CoV-2), or to one or more regions specific to an individual variant of a strain of virus (e.g., SARS-CoV-2 variant B.1.1.7, B.1.351, P.1, B.1427, B.1.429, B.1.526, B.1.525, or P.2).

One example of considering the detection goals and nature of the target at least partially single-stranded viral genome as outlined in Step 1 of FIG. 1A is the design of a general screen for influenza viruses to include multiple ID-Oligos (e.g., an array) that would be robust to the rapid evolution of influenza virus strains, due to mutation and re-assortment. Similarly, a general screen for HIV could involve multiple ID-Oligos that would be robust to the rapid evolution by point mutations and natural selection. In contrast, a number of highly specific ID-Oligos could be chosen to detect a specific strain of influenza that might help pinpoint vaccine priorities or might help identify emergence of drug resistance in strains of HIV. In an exemplary embodiment, FIG. 1B shows the theoretical range for PPi release per 1× copy of the target single-stranded viral genome, as a function of the template regions length (e.g., the length of a uracil/thymine-free region) and the number of oligonucleotide molecules complementary to a selected portion of a target at least partially single-stranded viral genome (i.e., ID-Oligo or “primer”) included with a the reaction mixture. In some embodiments of the methods and kits described herein, uracil/thymine-free regions are selected with their length in mind, because longer stretches will produce a stronger signal. This guiding process simplifies, streamlines, and accelerates the selection of regions to serve as the oligonucleotide molecules complementary to a selected portion of a target at least partially single-stranded viral genome (i.e., ID-Oligos).

In the exemplary embodiment of FIG. 1A, step 2 involves obtaining a sample (FIG. 1A). If a sample (e.g., a patient sample, water sample, food sample, animal product, etc.) contains free single-strand nucleic acid targets (e.g., ssRNA or ssDNA), it can be used directly. If a sample contains nucleic acid targets that are enclosed within full virions or capsids, then the nucleic acid targets would need to be extracted by one or more means, including those currently commercially available or those engineered into a ID-Oligo detection system. Thus, the assay according to methods described herein may be carried out using various biological fluids including, for example, plasma, serum, and saliva. In some embodiments, the assay according to methods described herein is carried out using tissue, cell, semen, vaginal fluid, sputum, and mucus samples. The biological samples may, for example, be collected on a swab (e.g., a nasal swab). Depending on the viral load/titer and the availability/concentrations of free nucleic acids in a given sample, the assay according to methods described herein described herein can be carried out using non-processed samples (e.g., detecting free/circulating RNA/DNA in the biological fluid) or isolated RNA/DNA (e.g., isolation can be achieved by any means of nucleic acid isolation including, without limitation, commercial kits, off-the-shelf reagents, and the like).

In some embodiments the sample is contacted with one or more reagents selected from the group consisting of phospholipid-disrupting detergents, enzymes that break down the viral membrane envelope (e.g., phospholipases, α-Hemolysin), and proteases which break open the viral capsid (e.g., Proteinase K). Such reagents may aid in the release of DNA/RNA from the viral envelope and/or virion. In accordance with such embodiments, tethering the enzymes to solid supports such as nanoparticles enables physical and spatial separation between the virus-lysing enzymes/reagents and the enzymes producing the bioluminescent readout. Thus, a built-in virus lysis step could be performed in different ways: (i) lysis and polymerization/luminescence may occur simultaneously and in the same space (e.g., in a common well or region of a device); (ii) lysis and polymerization/luminescence may occur in-line serially in a microfluidic or paper chromatography system (or similar system in which the sample containing viral RNA/DNA is delivered at an upstream site and then moved through the system via capillary action or flow generated by a pump or other system), with the lytic enzymes/reagents localized upstream of the ID-Oligos and enzymes producing luminescence; or (iii) lysis and polymerization/luminescence may occur separately and sequentially, where the lysis occurs in one step, and the isolated RNA/DNA is then added to the polymerization/luminescence reaction mixture in a separate device/container.

In the exemplary embodiment of FIG. 1A, step 3 involves the annealing of full or partial ssRNA/ssDNA (e.g., a viral genome from the sample or sample extract) with one or more ID-Oligos (each pre-designed to have a complementary sequence to the target domain within the viral genome as described herein) (FIG. 1A).

In the exemplary embodiment of FIG. 1A, step 4 involves the binding of reverse-transcriptase (RT) and/or DNA-Polymerase (depending on whether the template region is RNA or DNA) to the newly formed double strand RNA/DNA hybrid or dsDNA (FIG. 1A). Various polymerases can be used. To improve efficiency, kinetics, stability and/or positioning within a detection device, the polymerase may be tethered to a nanoparticle.

In the exemplary embodiment of FIG. 1A, step 5 involves the extension of complementary RNA/DNA strand from the hybridized ID-Oligos (FIG. 1A). The polymerization extension reaction may occur using available nucleotides provided in a dNTP mix added to the reaction buffer (dCTP/dGTP/dTTP). In this exemplary embodiment, lack of inclusion of dATP is made possible by earlier selection of the uracil/thymine-free regions. Alternatively, to generate longer regions for extension (increasing signal per genome), dATP analogs with lower ability to be hydrolyzed by luciferase can be used (e.g., thio-derivatives of dATP, dATPαS).

In the exemplary embodiment of FIG. 1A, step 6 involves the release of phosphate groups (PPi, 2 per nucleotide added to the ssRNA/ssDNA strand) by the polymerization extension reaction (FIG. 1A). A longer template region (e.g., a uracil/thymine-free region) will produce more PPi and a stronger signal.

In the exemplary embodiment of FIG. 1A, step 7 involves the conversion of released phosphates into ATP by downstream enzymatic reactions (e.g., in some embodiments, the enzymes are enzymes tethered to a support (e.g., a nanoparticle)) (FIG. 1A). In some embodiments, free phosphates and APS (adenosine 5′-phosphosulfate) are converted into ATP by ATP-sulfurylase. In some embodiments, free phosphates along with NAD/GAP/ADP are converted into ATP by the coupled reaction of GAPDH-PGK.

In the exemplary embodiment of FIG. 1A, step 8 involves luciferase (e.g., soluble luciferase or luciferase tethered to nanoparticles) metabolizing ATP to produce bioluminescence as the readout signal (FIG. 1A).

In the exemplary embodiment of FIG. 1A, step 9 involves quantifying the readout luminescence signal to determine the presence/relative abundance or concentration of the target viral genome (FIG. 1A).

Another embodiment is shown schematically in FIG. 4. In this embodiment, a polymerase (in this case, reverse transcriptase (RT)) is tethered to one nanoparticle (NP), and ATP sulfurylase (ATP-sul) and luciferase (Luc) are tethered to another nanoparticle. One or more virus-specific primer(s) (ID-Oligo(s)) anneal to the complementary sequence (single-strand ribonucleic acid (ssRNA) or single-strand deoxyribonucleic acid (ssDNA)) within a target viral genome (or portion or fragment thereof), and a double-strand (i.e., hybridization product) is generated. This allows the polymerase to bind and initiate the polymerization extension reaction, thereby releasing free phosphates (PPi) through nucleotide incorporation into the growing strand. Adenosine 5′-phosphosulfate (APS) is provided for the assay. In the presence of APS, the free phosphates are used by the tethered ATP-sul to generate ATP. In conjunction with luciferin, the ATP will then be used by tethered Luc to generate a bioluminescent signal. The amount of light emitted is directly proportional to the amount of ATP in the system, thereby corresponding to the amount of target viral genomic nucleic acid in the system. Light emitted may, in one embodiment, be read quantitatively and/or qualitatively by a photodetector positioned to capture that emitted signal. In certain embodiments described herein, the assay may be carried out within, e.g., a microfluidic channel of a device. Such a device may include a plurality of microfluidic channels, each channel containing components for an assay as described herein.

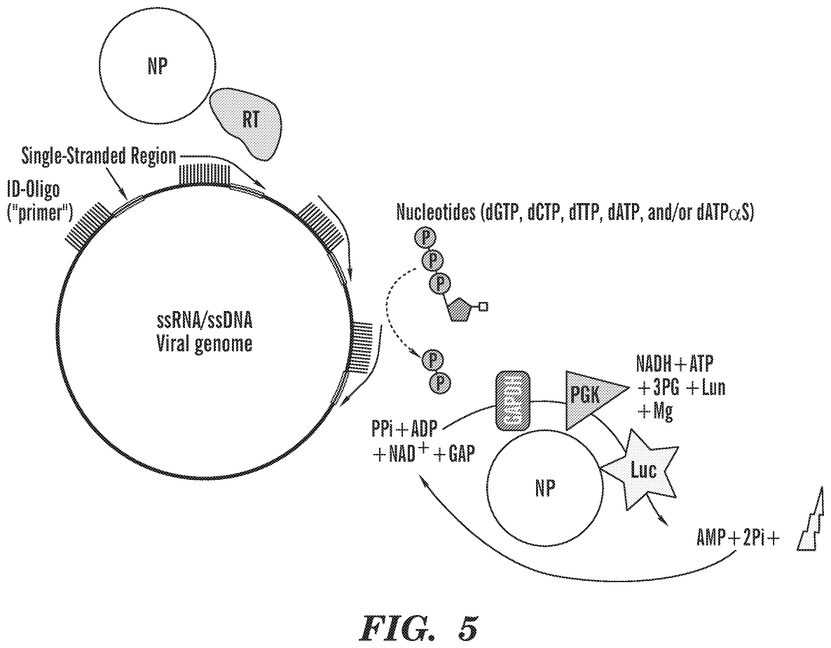

In a further embodiment, as shown in FIG. 5, a polymerase (in this case, reverse transcriptase (RT)) is tethered to one nanoparticle (NP) and glyceraldehyde 3-phosphate dehydrogenase (GAPDH), phosphoglycerate kinase (PGK), and luciferase (Luc) are tethered to another nanoparticle. One or more virus-specific primer(s) (ID-oligo(s)) anneal to the complementary sequence (single-strand ribonucleic acid (ssRNA) or single-strand deoxyribonucleic acid (ssDNA)) within a target viral genome (or portion or fragment thereof), and a double-strand (i.e., hybridization product) is generated. This allows the polymerase to bind and initiate the polymerization extension reaction, thereby releasing free phosphates (PPi) through nucleotide incorporation into the growing strand. ADP, NAD⁺, and glyceraldehyde 3-phosphate (GAP) are provided for the assay. In the presence of these components, the free phosphates are used by the tethered GAPDH and PGK in a coupled reaction to generate ATP. In conjunction with luciferin, the ATP will then be used by tethered Luc to generate a bioluminescent signal. The amount of light emitted is directly proportional to the amount of ATP in the system, thereby corresponding to the amount of target viral genomic nucleic acid in the system. Light emitted may, in one embodiment, be read quantitatively and/or qualitatively by a photodetector positioned to capture that emitted signal. In certain embodiments described herein, the assay may be carried out within, e.g., a microfluidic channel of a device. Such a device may include a plurality of microfluidic channels, each channel containing components for an assay as described herein.

The term “nucleic acid” refers to polymers of nucleotides (e.g., ribonucleotides and/or deoxyribonucleotides, both natural and non-natural) including DNA, RNA, and their subcategories, such as cDNA, mRNA, miRNA etc. A nucleic acid may be single-stranded and will generally contain 5′-3′ phosphodiester bonds, although in some cases, nucleotide analogs may have other linkages. Nucleic acids may include naturally occurring bases (adenine, guanine, cytosine, uracil, thymine) as well as non-natural bases.

As used herein, the terms “target at least partially single-stranded viral genome” or “target” refer to the nucleic acid sequence or portion or fragment thereof in a viral genome which is to be detected or analyzed. The term target includes all variants of the target at least partially single-stranded viral genome or target sequence, e.g., one or more viral variants and the wild type viral genome.

The target at least partially single-stranded viral genome (also referred to herein as the target, the target viral genome, or the like) may be any portion or fragment of a viral genome. In some embodiments, the target at least partially single-stranded viral genome is entirely single-stranded. In some embodiments, the target at least partially single-stranded viral genome is partially single-stranded.

The target viral genome may be from a virus that naturally possesses at least a partially single-stranded (ss) genome. The target viral genome may be a single-stranded RNA (positive or negative) viral genome or single-stranded DNA (positive or negative) viral genome.

The target viral genome may also be from a virus that naturally possesses a double-stranded RNA or DNA genome, which is subjected to a treatment to separate the strands of the double-stranded DNA to produce a single-strand from the double-stranded viral genome. The treatment used to separate the strands of the double-stranded DNA or RNA to produce a single-strand from the double-stranded viral genome may include, for example, contacting the double-stranded DNA or RNA with one or more a reagents (e.g., a helicase, topoisomerase, DNA polymerase, and endonuclease), thereby displacing one of the strands to produce a single-strand from the double-stranded viral genome. Such reagents may be provided and contacted with the sample prior to the methods described herein, or as part of the methods described herein. For example, in some embodiments, the step of processing the sample to, e.g., separate nucleic acids from other components of the sample may be carried out prior to providing the sample according to the methods described herein. In some embodiments, the step of processing the sample to, e.g., separate nucleic acids from other components of the sample may be carried out as part of the methods described herein. Such reagents may also be provided tethered to a solid support. Suitable solid supports are described herein. See FIGS. 4-5.

In some embodiments, the target viral genome is from a single-stranded positive-sense RNA virus (e.g., a virus from the family Arteriviridae, Coronaviridae, Flaviviridae, Togaviridae, Matonaviridae, Retroviridae, Caliciviridae, Hepeviridae, or Picornaviridae) or single-stranded negative-sense RNA virus (e.g., a virus from the family Arenaviridae, Deltavirus, Filoviridae, Orthomyxoviridae, Nairoviridae, Paramyxoviridae, Peribunyaviridae, Pneumoviridae, or Rhabdoviridae) (see, e.g., Payne S, “Introduction to RNA Viruses,” Viruses 97-105(2017), which is hereby incorporated by reference in its entirety). The RNA virus may be an enveloped or non-enveloped virus. Exemplary single-stranded RNA viruses include, without limitation, those identified in Table 1 below.

TABLE 1
Exemplary Single-Stranded RNA Viruses

| | | |
|---|---|---|
| Family | Genus/Genera | Exemplary Viruses |

Single-Stranded Positive-Sense Enveloped RNA Viruses

| | | |
|---|---|---|
| Coronaviridae | Alphacoronavirus | Human coronavirus 229E, Human coronavirus |
| | | NL63, Human coronavirus 2JL14 |
| | Betacoronavirus | Human coronavirus OC43, Human coronavirus |
| | | HKU1, Severe Acute Respiratory Syndrome |
| | | Coronavirus (SARS-CoV), Severe Acute |
| | | Respiratory Syndrome Coronavirus 2 (SARS-CoV- |
| | | 2), Severe Acute Respiratory Syndrome |
| | | Coronavirus Urbani (SARS-CoV-Urbani), Middle |
| | | East Respiratory Syndrome Coronavirus (MERS- |
| | | CoV) |
| Flaviviridae | Flavivirus | Yellow Fever virus, Dengue virus 1, Dengue virus |
| | | 2, Dengue virus 3, Dengue virus 4, Japanese |
| | | Encephalitis virus, West Nile virus, Kunjin virus, |
| | | St. Louis encephalitis virus, Murray Valley |
| | | encephalitis virus, Usutu virus, Koutango virus, |
| | | Zika virus, Tick-borne encephalitis, Kyasanur |
| | | Forest disease virus, Alkhurma hemorrhagic fever |
| | | virus, Omsk hemorrhagic fever virus, Powassan |
| | | virus, Aroa virus, Kadam virus, Gadgets Gully |
| | | virus, Langat virus, Louping ill virus, Koutango |
| | | virus, Alfuy virus, Usutu virus, Stratford virus, |
| | | Kokobera virus, Cacipacoré virus, Yaoundé virus, |
| | | Tembusu virus, Ntaya virus, Bagaza virus, Ilhéus |
| | | virus, Sepik virus, Wesselsbron virus, Kédougou |
| | | virus, Banzi virus, Bouboui virus, Edge Hill virus, |
| | | Jugra virus, Potiskum virus, Saboya virus, Ugando |
| | | S virus |
| | Hepacivirus | Hepatitis C virus |
| | Pegivirus | Human pegivirus genotype 1, Human pegivirus |
| | | genotype 2, Human pegivirus genotype 3, Human |
| | | pegivirus genotype 4, Human pegivirus genotype |
| | | 5, Human pegivirus genotype 6 |
| Togaviridae | Alphavirus | Aura Virus, Chikungunya virus, Eastern equine |
| | | encephalitis virus, Western equine encephalitis |
| | | virus, Venezuelan equine encephalitis virus, |
| | | Semliki Forest virus, Sindbis virus, Ross River |
| | | virus, Rio Negro virus, Everglades virus, |
| | | Middelburg virus, Barmah Forest virus, Una virus, |
| | | Sagiyama virus, Ndumu virus |
| Matonaviridae | Rubivirus | Rubella virus |
| Retroviridae | Deltaretrovirus | Human T-lymphotropic virus (HTLV) (e.g., |
| | | HTLV1, HTLV2, HTLV3, HTLV4) |
| | Lentivirus | Human immunodeficiency virus (HIV) (e.g., HIV1, |
| | | HIV2) |
| | Spumavirus* | Human foamy virus |

Single-Stranded Positive-Sense Non-Enveloped RNA Viruses

| | | |
|---|---|---|
| Caliciviridae | Norovirus | Norwalk virus, Lordsdale virus, Maryland virus, |
| | | Jena virus, Alphatorn virus |
| | Sapovirus | Sapporo virus, Manchester virus, Bristol virus, |
| | | Cowden I virus, Ehime virus, Nongkhai virus |
| Hepeviridae | Orthohepevirus | Hepatitis E virus |
| Picornaviridae | Aphthovirus | Foot-and-mouth disease virus |
| | Cardiovirus | Scaffold virus |
| | Cosavirus | Cosavirus |
| | Enterovirus | Coxsackievirus, Enterovirus, Rhinovirus, |
| | | Poliovirus |
| | Hepatovirus | Hepatovirus, Hepatitis A |
| | Parechovirus | Parechovirus |

Single-Stranded Negative-Sense Enveloped RNA Viruses

| | | |
|---|---|---|
| Arenaviridae | Mammarenavirus | Lassa virus, Lujo virus, Junín virus, Machupo |
| | | virus, Guanarito virus, Sabiá virus, Chapare virus, |
| | | Flexal virus, lymphocytic choriomeningitis virus |
| Deltavirus | Deltavirus | Hepatitis delta virus |
| Filoviridae | Ebolavirus | Bombali virus, Bundibugyo virus, Reston virus, |
| | | Sudan virus, Taï Forest virus, Ebola virus |
| | Marburgvirus | Marburg virus, Ravn virus |
| Orthomyxoviridae | Influenzavirus A | Influenza A virus (e.g, H1N1, H2N2, H3N2) |
| | Influenzavirus B | Influenza B virus |
| | Influenzavirus C | Influenza C virus |
| | Thogotovirus | Thogoto virus, Dhori virus |
| Nairoviridae | Orthonairovirus | Artashat virus, Chim virus, Crimean-Congo |
| | | Hemorrhagic Fever virus, Dera Ghazi Khan virus, |
| | | Abu Hammad virus, Abu Mina virus, Sapphire II |
| | | virus, Dugbe virus, Estero Real virus, Hazara virus, |
| | | Farallon virus, Hughes virus, Keterah virus |
| Paramyxoviridae | Respirovirus | Human Parainfluenza virus 1, Human |
| | | Parainfluenza virus 3 |
| | Orthorubulavirus | Human Parainfluenza virus 2, Human |
| | | Parainfluenza virus 4, Mumps virus, Alston virus |
| | Henipavirus | Hendravirus, Nipavirus |
| | Orthoavulavirus | Newcastle disease virus |
| | Morbillivirus | Measles virus |
| Peribunyaviridae | Orthobunyavirus | La Crosse virus, Bunyamwera virus, Ngari virus, |
| | | Keystone virus, South River virus, Jerry Slough |
| | | virus, Kaeng Khoi virus, Kairi virus, Leanyer virus, |
| | | Lumbo virus, Madrid virus, Playas virus, Main |
| | | Drain virus, Manzanila virus, Inini virus, Marituba |
| | | virus, Jamestown Canyon virus, Ingwavuma virus, |
| | | Ilesha virus, laco virus, Guaroa virus, Guamá virus, |
| | | Guajará virus, Cát Qu [symbol] virus, Oya virus, Catú |
| | | virus, Enseada virus, Capim virus, California |
| | | encephalitis virus, Cache Valley virus, Bwamba |
| | | virus, Buttonwillow virus, Bushbush virus, Bimti |
| | | virus, Birao virus, Botambi virus, Bozo virus, Batai |
| | | virus, Batama virus, Bellavista virus, Benevides |
| | | virus, Bertioga virus, Anhembi virus, Lukuni virus, |
| | | Aino virus, Akabane virus, Acará virus |
| Pneumoviridae | Metapneumoivirus | Human metapneumovirus |
| | Orthopneumovirus | Human orthopneumovirus, Human respiratory |
| | | syncytial virus |
| Rhabdoviridae | Lyssavirus | Aravan virus, Rabies virus, Mokola virus, Khujand |
| | | virus, Irkut virus, Ikoyama lyssavirus, Duvenhage |
| | | virus |
| | Barhavirus | Bahia Grande virus, Muir Springs virus |
| | Ledantevirus | Le Dantec virus, Nkolbisson virus |
| | Vesiculovirus | Isfahan virus, Piry virus, Vesicular stomatitis virus, |
| | | Chandipura virus, Malpais Spring virus, Maraba |
| | | virus, Morreton virus, Perinet virus, Radi virus, |
| | | Yug Bogdanovac virus |

*Spumaviruses may contain a RNA and/or DNA genome within the virion (see, e.g., Strauss & Strauss, “Chapter 6—Viruses Whose Life Cycle Uses Reverse Transcriptase,” in Viruses and Human Disease (Second Edition), pp. 211-259 (2008), which is hereby incorporated by reference in its entirety).

In some embodiments, the target viral genome is from a single-stranded DNA virus (e.g., a virus from the family Circoviridae, Parvoviridae, or Redondoviridae). Exemplary single-stranded DNA viruses include, without limitation, those identified in Table 2 below.

TABLE 2
Exemplary Single-Stranded DNA Viruses

| Family | Genus | Exemplary Viruses |
|---|---|---|
| Circoviridae | Circovirus | Human associated circovirus |
| | Cyclovirus | Human associated cyclovirus |
| Parvoviridae | Bocaparvovirus | Human bocavirus |
| | Dependoparvovirus | Adeno-associated virus |
| | Erythroparvovirus | Human parvovirus B19 |
| | Protoparvovirus | Bufavirus 1a, Cutavirus |
| | Tetraparvovirus | Human Parvovirus 4 |
| Redondoviridae | Torbevirus | Human respiratory-associated |
| | | brisavirus, Human lung- |
| | | associated brisavirus, |
| | | Human gut-associated |
| | | brisavirus, Human oral- |
| | | associated brisavirus |

Thus, in some embodiments, the target viral genome is from a virus selected from the group consisting of an alphacoronavirus, a betacoronavirus, a flavivirus, a hepacivirus, a pegivirus, an alphavirus, a rubivirus, a deltaretrovirus, a lentivirus, a spumavirus, a norovirus, a sapovirus, an orthohepevirus a aphthovirus, a cardiovirus, a cosavirus, an enterovirus, a hepatovirus, a parechovirus, a mammarenavirus, a deltavirus, an ebolavirus, a marburgvirus, an influenzavirus A, an influenzavirus B, an influenzavirus C, a thogotovirus, an orthonairovirus, a respirovirus, an orthorubulavirus, a henipavirus, an orthoavulavirus, a morbillivirus, an orthobunyavirus, an orthobunyavirus, a metapneumoivirus, an orthopneumovirus, a lyssavirus, a barhavirus, a ledantevirus, a vesiculovirus, a circovirus, a cyclovirus, a bocaparvovirus, a dependoparvovirus, an erythroparvovirus, a protoparvovirus, a tetraparvovirus, and/or a torbevirus. For example, the target viral genome may be from a virus selected from the group consisting of Severe Acute Respiratory Syndrome Coronavirus 2 (SARS-CoV-2), Severe Acute Respiratory Syndrome Coronavirus Urbani (SARS-CoV-Urbani), Middle East Respiratory Syndrome Coronavirus (MERS-CoV), Yellow Fever virus, Dengue virus 1, Dengue virus 2, Dengue virus 3, Dengue virus 4, Japanese Encephalitis virus, West Nile virus, Zika virus, Hepatitis C virus, Chikungunya virus, Ross River virus, Sindbis virus, Rubella virus, Norwalk virus, Coxsackievirus, Enterovirus, Rhinovirus, Poliovirus, Ebola virus, Lassa virus, Marburg virus, Hendravirus, Nipavirus, Newcastle disease virus, Parainfluenza virus, Mumps virus, Measles virus, Rabies virus, Human respiratory syncytial virus, Human parvovirus B19, Foot-and-mouth disease virus, Vesicular stomatitis virus, Human Immunodeficiency virus, Influenzavirus, and/or Hepatitis A virus.

In some embodiments, the target viral genome is from a double-stranded RNA virus (e.g., a virus from the family Picobirnaviridae). The double-stranded RNA virus may be an enveloped or non-enveloped (e.g., a virus from the family Picobirnaviridae) virus. Exemplary double-stranded RNA viruses include, without limitation, those identified in Table 3 below.

In some embodiments, the target viral genome is from a double-stranded DNA virus (e.g., a virus from the family Papillomaviridae). The double-stranded DNA virus may be an enveloped (e.g., a virus from the family Herpesviridae or Hepadnaviridae) or a non-enveloped (e.g., a virus from the family Papillomaviridae or Polyomaviridae) virus. Exemplary double-stranded DNA viruses include, without limitation, those identified in Table 3 below.

TABLE 3
Exemplary Double-Stranded Viruses

| | | |
|---|---|---|
| Family | Genus/Genera | Exemplary Viruses |

Double-Stranded RNA Viruses

| | | |
|---|---|---|
| Picobirnaviridae | Picobirnavirus | Human picobirnavirus |

Double-Stranded DNA Viruses

| | | |
|---|---|---|
| Papillomaviridae | Alphapapillomavirus | Human papillomavirus (HPV) (e.g., HPV 32, HPV |
| | | 42, HPV 6, HPV 10, HPV 11, HPV 13, HPV 16, |
| | | HPV 160, HPV 94, HPV 125, HPV 117) |
| | Betapapillomavirus | Human papillomavirus (HPV) (e.g, HPV 5, |
| | | HPV8, HPV 93, HPV 98, HPV 99, HPV 105, |
| | | HPV 118) |
| Polyomaviridae | Alphapolyomavirus | Merkel cell polyomavirus (e.g., Human |
| | | polyomavirus 5), Trichodysplasia spinulosa- |
| | | associated polyomavirus, Human polyomavirus 9, |
| | | New Jersey polyomavirus, LI polyomavirus) |
| | Betapolyomavirus | BK polyomavirus, JC polyomavirus, KI |
| | | polyomavirus, WU polyomavirus) |
| | Deltapolyomavirus | human polyomavirus 6, human polyomavirus 7, |
| | | MW polyomavirus, STL polyomavirus |
| Herpesviridae | Cytomegalovirus | Human cytomegalovirus (HCMV, Human |
| | | herpesvirus 5) |
| | Rhadinovirus | Kaposi’s sarcoma-associated herpesvirus (KSHV, |
| | | Human gammaherpesvirus 8, HHV-8) |
| | Roseolovirus | Human herpesvirus 6 (e.g, HHV6A, HHV6A, |
| | | HHV6B), Human herpesvirus 7 (HHV7) |
| | Simplexvirus | Herpes simplex virus (HSV) (e.g., HSV1, HSV2) |
| | Lymphocryptovirus | Epstein-Barr virus (EBV, Human herpesvirus 4, |
| | | HHV4) |
| | Varicellovirus | Human herpesvirus 3 (HHV3, Varicella-zoster |
| | | virus, VZV) |
| Hepadnaviridae* | Orthohepadnavirus | Hepatitis B virus |

*The hepadnavirus genome consists of a partially dsDNA held in a circular conformation by base pairing in a cohesive overlap between the 5′-ends of the two DNA strands (Seeger and Mason, “Molecular Biology of Hepatitis B Virus Infection,” Virology (479-480): 672-686 (2015), which is hereby incorporated by reference in its entirety).

Thus, in some embodiments, the target viral genome is from a virus selected from the group consisting of a picobirnavirus, an alphapapillomavirus, a betapapillomavirus, an alphapolyomavirus, a betapolyomavirus, a deltapolyomavirus, a cytomegalovirus, a rhadinovirus, a roseolovirus, a simplexvirus, a lymphocryptovirus, a varicellovirus, and/or an orthohepadnavirus. For example, the target viral genome may be from a virus selected from the group consisting of Human papillomavirus, Merkel cell polyomavirus, Hyman cytomegalovirus, Kaposi's sarcoma virus, Human herpesvirus 6, Human herpesvirus 7, Herpes simplex virus, Epstein-Barr virus, Human herpesvirus 3, and/or Hepatitis B virus.

In one embodiment, the target viral genome is from SARS-CoV-2. SARS-CoV-2 is the positive-sense RNA virus that causes coronavirus disease 2019 (COVID-19). The genomic structure of SARS-CoV-2 is characteristic of other known coronaviruses. In particular, more than two-thirds of the genome comprises the ORF lab region (comprising ORF1a and ORF1b), which is located at the 5′ end of the genome and encodes ORF1ab polyproteins. The remaining one third of the genome, located 3′ to the ORF1ab region, consists of genes encoding structural proteins including surface (S), envelope (E), membrane (M), and nucleocapsid (N) proteins. Additionally, the SARS-CoV-2 contains six accessory proteins, encoded by ORF3a, ORF6, ORF7a, ORF7b, and ORF8 regions. (Khailany et al., “Genomic characterization of a novel SARS-CoV-2,” Gene Rep. Apr. 16, 2020, which is hereby incorporated by reference in its entirety.)

The first sequence of the SARS-CoV-2 genome isolated from Wuhan was deposited as NCBI Reference Sequence No. NC_045512.2 and GenBank Ref. No. MN908947, which are hereby incorporated by reference in their entirety (see Table 9). Thus, in some embodiments, the target viral genome has the sequence corresponding to that of SEQ ID NO: 1, which is shown in Table 9 (Wu et al., “A New Coronavirus Associated with Human Respiratory Disease in China,” Nature 579(7798):265-269 (2020), which are hereby incorporated by reference in its entirety). It will be understood that RNA sequences provided herein, referenced herein, or reported in public databases, e.g., GenBank, will list or report thymine (i.e., T) rather than uracil (i.e., U), which is based on RNA sequencing techniques that involve sequencing of, e.g., cDNA. Thus, it will be understood that any RNA genome sequence (or portion thereof) provided herein or reported in referenced public databases is contemplated as possessing uracil(s) in place of the identified thymine(s).

The primers (or ID-Oligos) described herein may be directed to any portion of the target viral genome or a fragment thereof. For instance, the primer or multiple primers (or ID-Oligos) described herein may be directed to one or more of the nucleic acid sequences encoding structural proteins including surface protein, envelope protein, membrane protein, and nucleocapsid protein, and/or the six accessory proteins, encoded by ORF3a, ORF6, ORF7a, ORF7b and ORF8 regions of a SARS-CoV-2 target viral genome. For example, the primers (or ID-Oligos) described herein may be directed to a portion of the SARS-CoV-2 target viral genome of SEQ ID NO:1 (or a variant thereof) encoding a structural protein such as surface protein (e.g., nucleic acid residues 21,563-25,384 of SEQ ID NO:1), envelope protein (e.g., nucleic acid residues 26,245-26,472 of SEQ ID NO:1), membrane protein (e.g., nucleic acid residues 26,523-27,191 of SEQ ID NO:1), and nucleocapsid protein (e.g., nucleic acid residues 28,274-29,533 of SEQ ID NO:1), and/or one or more of the six accessory proteins, encoded by ORF3a (e.g., nucleic acid residues 25,393-26,220 of SEQ ID NO:1), ORF6 (e.g., nucleic acid residues 27,202-27,387 of SEQ ID NO:1), ORF7a (e.g., nucleic acid residues 27,394-27,759 of SEQ ID NO:1), ORF7b (e.g., nucleic acid residues 27,756-27,887 of SEQ ID NO:1), and ORF8 regions (e.g., nucleic acid residues 27,894-28,259 of SEQ ID NO:1).

The encoded proteins for the SARS-CoV-2 target viral genome of SEQ ID NO:1 are also identified in Table 4 below.

TABLE 4
Exemplary SARS-Cov-2 Protein Sequences (GenBank Ref. No. MN908947, which is
hereby incorporated by reference in its entirety)

| | | SEQ |
|---|---|---|
| Protein Name | Amino Acid Sequence | ID NO: |
| Spike Protein | MFVFLVLLPLVSSQCVNLTTRTQLPPAYTNSFTRGVYYPDKVFR | 280 |
| | SSVLHSTQDLFLPFFSNVTWFHAIHVSGTNGTKRFDNPVLPFND | |
| | GVYFASTEKSNIIRGWIFGTTLDSKTQSLLIVNNATNVVIKVCE | |
| | FQFCNDPFLGVYYHKNNKSWMESEFRVYSSANNCTFEYVSQPFL | |
| | MDLEGKQGNFKNLREFVFKNIDGYFKIYSKHTPINLVRDLPQGF | |
| | SALEPLVDLPIGINITRFQTLLALHRSYLTPGDSSSGWTAGAAA | |
| | YYVGYLQPRTFLLKYNENGTITDAVDCALDPLSETKCTLKSFTV | |
| | EKGIYQTSNFRVQPTESIVRFPNITNLCPFGEVFNATRFASVYA | |
| | WNRKRISNCVADYSVLYNSASFSTFKCYGVSPTKLNDLCFTNVY | |
| | ADSFVIRGDEVRQIAPGQTGKIADYNYKLPDDFTGCVIAWNSNN | |
| | LDSKVGGNYNYLYRLFRKSNLKPFERDISTEIYQAGSTPCNGVE | |
| | GFNCYFPLQSYGFQPTNGVGYQPYRVVVLSFELLHAPATVCGPK | |
| | KSTNLVKNKCVNFNFNGLTGTGVLTESNKKFLPFQQFGRDIADT | |
| | TDAVRDPQTLEILDITPCSFGGVSVITPGTNTSNQVAVLYQDVN | |
| | CTEVPVAIHADQLTPTWRVYSTGSNVFQTRAGCLIGAEHVNNSY | |
| | ECDIPIGAGICASYQTQTNSPRRARSVASQSIIAYTMSLGAENS | |
| | VAYSNNSIAIPTNFTISVTTEILPVSMTKTSVDCTMYICGDSTE | |
| | CSNLLLQYGSFCTQLNRALTGIAVEQDKNTQEVFAQVKQIYKTP | |
| | PIKDFGGFNFSQILPDPSKPSKRSFIEDLLFNKVTLADAGFIKQ | |
| | YGDCLGDIAARDLICAQKFNGLTVLPPLLTDEMIAQYTSALLAG | |
| | TITSGWTFGAGAALQIPFAMQMAYRFNGIGVTQNVLYENQKLIA | |
| | NQFNSAIGKIQDSLSSTASALGKLQDVVNQNAQALNTLVKQLSS | |
| | NFGAISSVLNDILSRLDKVEAEVQIDRLITGRLQSLQTYVTQQL | |
| | IRAAEIRASANLAATKMSECVLGQSKRVDFCGKGYHLMSFPQSA | |
| | PHGVVFLHVTYVPAQEKNFTTAPAICHDGKAHFPREGVFVSNGT | |
| | HWFVTQRNFYEPQIITTDNTFVSGNCDVVIGIVNNTVYDPLQPE | |
| | LDSFKEELDKYFKNHTSPDVDLGDISGINASVVNIQKEIDRLNE | |
| | VAKNLNESLIDLQELGKYEQYIKWPWYIWLGFIAGLIAIVMVTI | |
| | MLCCMTSCCSCLKGCCSCGSCCKFDEDDSEPVLKGVKLHYT | |
| Nucleocapsid | MSDNGPQNQRNAPRITFGGPSDSTGSNQNGERSGARSKQRRPQG | 281 |
| Protein | LPNNTASWFTALTQHGKEDLKFPRGQGVPINTNSSPDDQIGYYR | |
| | RATRRIRGGDGKMKDLSPRWYFYYLGTGPEAGLPYGANKDGIIW | |
| | VATEGALNTPKDHIGTRNPANNAAIVLQLPQGTTLPKGFYAEGS | |
| | RGGSQASSRSSSRSRNSSRNSTPGSSRGTSPARMAGNGGDAALA | |
| | LLLLDRLNQLESKMSGKGQQQQGQTVTKKSAAEASKKPRQKRTA | |
| | TKAYNVTQAFGRRGPEQTQGNFGDQELIRQGTDYKHWPQIAQFA | |
| | PSASAFFGMSRIGMEVTPSGTWLTYTGAIKLDDKDPNFKDQVIL | |
| | LNKHIDAYKTFPPTEPKKDKKKKADETQALPQRQKKQQTVTLLP | |
| | AADLDDFSKQLQQSMSSADSTQA | |
| Envelope | MYSFVSEETGTLIVNSVLLFLAFVVFLLVTLAILTALRLCAYCC | 282 |
| Protein | NIVNVSLVKPSFYVYSRVKNLNSSRVPDLLV | |
| Membrane | MADSNGTITVEELKKLLEQWNLVIGFLFLTWICLLQFAYANRNR | 283 |
| Protein | FLYIIKLIFLWLLWPVTLACFVLAAVYRINWITGGIAIAMACLV | |
| | GLMWLSYFIASFRLFARTRSMWSFNPETNILLNVPLHGTILTRP | |
| | LLESELVIGAVILRGHLRIAGHHLGRCDIKDLPKEITVATSRTL | |
| | SYYKLGASQRVAGDSGFAAYSRYRIGNYKLNTDHSSSSDNIALL | |
| | VQ | |
| ORF lab | MESLVPGFNEKTHVQLSLPVLQVRDVLVRGFGDSVEEVLSEARQ | 284 |
| Polyprotein | HLKDGTCGLVEVEKGVLPQLEQPYVFIKRSDARTAPHGHVMVEL | |
| | VAELEGIQYGRSGETLGVLVPHVGEIPVAYRKVLLRKNGNKGAG | |
| | GHSYGADLKSFDLGDELGTDPYEDFQENWNTKHSSGVTRELMRE | |
| | LNGGAYTRYVDNNFCGPDGYPLECIKDLLARAGKASCTLSEQLD | |
| | FIDTKRGVYCCREHEHEIAWYTERSEKSYELQTPFEIKLAKKFD | |
| | TFNGECPNFVFPLNSIIKTIQPRVEKKKLDGFMGRIRSVYPVAS | |
| | PNECNQMCLSTLMKCDHCGETSWQTGDFVKATCEFCGTENLTKE | |
| | GATTCGYLPQNAVVKIYCPACHNSEVGPEHSLAEYHNESGLKTI | |
| | LRKGGRTIAFGGCVFSYVGCHNKCAYWVPRASANIGCNHTGVVG | |
| | EGSEGLNDNLLEILQKEKVNINIVGDFKLNEEIAIILASESAST | |
| | SAFVETVKGLDYKAFKQIVESCGNFKVTKGKAKKGAWNIGEQKS | |
| | ILSPLYAFASEAARVVRSIFSRTLETAQNSVRVLQKAAITILDG | |
| | ISQYSLRLIDAMMFTSDLATNNLVVMAYITGGVVQLTSQWLTNI | |
| | FGTVYEKLKPVLDWLEEKFKEGVEFLRDGWEIVKEISTCAGEIV | |
| | GGQIVTCAKEIKESVQTFFKLVNKFLALCADSIIIGGAKLKALN | |
| | LGETFVTHSKGLYRKCVKSREETGLLMPLKAPKEIIFLEGETLP | |
| | TEVLTEEVVLKTGDLQPLEQPTSEAVEAPLVGTPVCINGLMLLE | |
| | IKDTEKYCALAPNMMVTNNTFTLKGGAPTKVTFGDDTVIEVQGY | |
| | KSVNITFELDERIDKVLNEKCSAYTVELGTEVNEFACVVADAVI | |
| | KTLQPVSELLTPLGIDLDEWSMATYYLFDESGEFKLASHMYCSF | |
| | YPPDEDEEEGDCEEEEFEPSTQYEYGTEDDYQGKPLEFGATSAA | |
| | LQPEEEQEEDWLDDDSQQTVGQQDGSEDNQTTTIQTIVEVQPQL | |
| | EMELTPVVQTIEVNSFSGYLKLTDNVYIKNADIVEEAKKVKPTV | |
| | VVNAANVYLKHGGGVAGALNKATNNAMQVESDDYIATNGPLKVG | |
| | GSCVLSGHNLAKHCLHVVGPNVNKGEDIQLLKSAYENFNQHEVL | |
| | LAPLLSAGIFGADPIHSLRVCVDTVRTNVYLAVFDKNLYDKLVS | |
| | SFLEMKSEKQVEQKIAEIPKEEVKPFITESKPSVEQRKQDDKKI | |
| | KACVEEVTTTLEETKFLTENLLLYIDINGNLHPDSATLVSDIDI | |
| | TFLKKDAPYIVGDVVQEGVLTAVVIPTKKAGGTTEMLAKALRKV | |
| | PTDNYITTYPGQGLNGYTVEEAKTVLKKCKSAFYILPSIISNEK | |
| | QEILGTVSWNLREMLAHAEETRKLMPVCVETKAIVSTIQRKYKG | |
| | IKIQEGVVDYGARFYFYTSKTTVASLINTLNDLNETLVTMPLGY | |
| | VTHGLNLEEAARYMRSLKVPATVSVSSPDAVTAYNGYLTSSSKT | |
| | PEEHFIETISLAGSYKDWSYSGQSTQLGIEFLKRGDKSVYYTSN | |
| | PTTFHLDGEVITFDNLKTLLSLREVRTIKVFTTVDNINLHTQVV | |
| | DMSMTYGQQFGPTYLDGADVTKIKPHNSHEGKTFYVLPNDDTLR | |
| | VEAFEYYHTTDPSFLGRYMSALNHTKKWKYPQVNGLTSIKWADN | |
| | NCYLATALLTLQQIELKFNPPALQDAYYRARAGEAANFCALILA | |
| | YCNKTVGELGDVRETMSYLFQHANLDSCKRVLNVVCKTCGQQQT | |
| | TLKGVEAVMYMGTLSYEQFKKGVQIPCTCGKQATKYLVQQESPF | |
| | VMMSAPPAQYELKHGTFTCASEYTGNYQCGHYKHITSKETLYCI | |
| | DGALLTKSSEYKGPITDVFYKENSYTTTIKPVTYKLDGVVCTEI | |
| | DPKLDNYYKKDNSYFTEQPIDLVPNQPYPNASFDNFKFVCDNIK | |
| | FADDLNQLTGYKKPASRELKVTFFPDLNGDVVAIDYKHYTPSFK | |
| | KGAKLLHKPIVWHVNNATNKATYKPNTWCIRCLWSTKPVETSNS | |
| | FDVLKSEDAQGMDNLACEDLKPVSEEVVENPTIQKDVLECNVKT | |
| | TEVVGDIILKPANNSLKITEEVGHTDLMAAYVDNSSLTIKKPNE | |
| | LSRVLGLKTLATHGLAAVNSVPWDTIANYAKPFLNKVVSTTTNI | |
| | VTRCLNRVCTNYMPYFFTLLLQLCTFTRSTNSRIKASMPTTIAK | |
| | NTVKSVGKFCLEASFNYLKSPNFSKLINIIIWFLLLSVCLGSLI | |
| | YSTAALGVLMSNLGMPSYCTGYREGYLNSTNVTIATYCTGSIPC | |
| | SVCLSGLDSLDTYPSLETIQITISSFKWDLTAFGLVAEWFLAYI | |
| | LFTRFFYVLGLAAIMQLFFSYFAVHFISNSWLMWLIINLVQMAP | |
| | ISAMVRMYIFFASFYYVWKSYVHVVDGCNSSTCMMCYKRNRATR | |
| | VECTTIVNGVRRSFYVYANGGKGFCKLHNWNCVNCDTFCAGSTF | |
| | ISDEVARDLSLQFKRPINPTDQSSYIVDSVTVKNGSIHLYFDKA | |
| | GQKTYERHSLSHFVNLDNLRANNTKGSLPINVIVFDGKSKCEES | |
| | SAKSASVYYSQLMCQPILLLDQALVSDVGDSAEVAVKMFDAYVN | |
| | TFSSTFNVPMEKLKTLVATAEAELAKNVSLDNVLSTFISAARQG | |
| | FVDSDVETKDVVECLKLSHQSDIEVTGDSCNNYMLTYNKVENMT | |
| | PRDLGACIDCSARHINAQVAKSHNIALIWNVKDFMSLSEQLRKQ | |
| | IRSAAKKNNLPFKLTCATTRQVVNVVTTKIALKGGKIVNNWLKQ | |
| | LIKVTLVFLFVAAIFYLITPVHVMSKHTDFSSEIIGYKAIDGGV | |
| | TRDIASTDTCFANKHADFDTWFSQRGGSYTNDKACPLIAAVITR | |
| | EVGFVVPGLPGTILRTTNGDFLHFLPRVFSAVGNICYTPSKLIE | |
| | YTDFATSACVLAAECTIFKDASGKPVPYCYDTNVLEGSVAYESL | |
| | RPDTRYVLMDGSIIQFPNTYLEGSVRVVTTFDSEYCRHGTCERS | |
| | EAGVCVSTSGRWVLNNDYYRSLPGVFCGVDAVNLLTNMFTPLIQ | |
| | PIGALDISASIVAGGIVAIVVTCLAYYFMRFRRAFGEYSHVVAF | |
| | NTLLFLMSFTVLCLTPVYSFLPGVYSVIYLYLTFYLTNDVSFLA | |
| | HIQWMVMFTPLVPFWITIAYIICISTKHFYWFFSNYLKRRVVFN | |
| | GVSFSTFEEAALCTFLLNKEMYLKLRSDVLLPLTQYNRYLALYN | |
| | KYKYFSGAMDTTSYREAACCHLAKALNDFSNSGSDVLYQPPQTS | |
| | ITSAVLQSGFRKMAFPSGKVEGCMVQVTCGTTTLNGLWLDDVVY | |
| | CPRHVICTSEDMLNPNYEDLLIRKSNHNFLVQAGNVQLRVIGHS | |
| | MQNCVLKLKVDTANPKTPKYKFVRIQPGQTFSVLACYNGSPSGV | |
| | YQCAMRPNFTIKGSFLNGSCGSVGFNIDYDCVSFCYMHHMELPT | |
| | GVHAGTDLEGNFYGPFVDRQTAQAAGTDTTITVNVLAWLYAAVI | |
| | NGDRWFLNRFTTTLNDFNLVAMKYNYEPLTQDHVDILGPLSAQT | |
| | GIAVLDMCASLKELLQNGMNGRTILGSALLEDEFTPFDVVRQCS | |
| | GVTFQSAVKRTIKGTHHWLLLTILTSLLVLVQSTQWSLFFFLYE | |
| | NAFLPFAMGIIAMSAFAMMFVKHKHAFLCLFLLPSLATVAYFNM | |
| | VYMPASWVMRIMTWLDMVDTSLSGFKLKDCVMYASAVVLLILMT | |
| | ARTVYDDGARRVWTLMNVLTLVYKVYYGNALDQAISMWALIISV | |
| | TSNYSGVVTTVMFLARGIVFMCVEYCPIFFITGNTLQCIMLVYC | |
| | FLGYFCTCYFGLFCLLNRYFRLTLGVYDYLVSTQEFRYMNSQGL | |
| | LPPKNSIDAFKLNIKLLGVGGKPCIKVATVQSKMSDVKCTSVVL | |
| | LSVLQQLRVESSSKLWAQCVQLHNDILLAKDTTEAFEKMVSLLS | |
| | VLLSMQGAVDINKLCEEMLDNRATLQAIASEFSSLPSYAAFATA | |
| | QEAYEQAVANGDSEVVLKKLKKSLNVAKSEFDRDAAMQRKLEKM | |
| | ADQAMTQMYKQARSEDKRAKVTSAMQTMLFTMLRKLDNDALNNI | |
| | INNARDGCVPLNIIPLTTAAKLMVVIPDYNTYKNTCDGTTFTYA | |
| | SALWEIQQVVDADSKIVQLSEISMDNSPNLAWPLIVTALRANSA | |
| | VKLQNNELSPVALRQMSCAAGTTQTACTDDNALAYYNTTKGGRF | |
| | VLALLSDLQDLKWARFPKSDGTGTIYTELEPPCRFVTDTPKGPK | |
| | VKYLYFIKGLNNLNRGMVLGSLAATVRLQAGNATEVPANSTVLS | |
| | FCAFAVDAAKAYKDYLASGGQPITNCVKMLCTHTGTGQAITVTP | |
| | EANMDQESFGGASCCLYCRCHIDHPNPKGFCDLKGKYVQIPTTC | |
| | ANDPVGFTLKNTVCTVCGMWKGYGCSCDQLREPMLQSADAQSFL | |
| | NRVCGVSAARLTPCGTGTSTDVVYRAFDIYNDKVAGFAKFLKTN | |
| | CCRFQEKDEDDNLIDSYFVVKRHTFSNYQHEETIYNLLKDCPAV | |
| | AKHDFFKFRIDGDMVPHISRQRLTKYTMADLVYALRHFDEGNCD | |
| | TLKEILVTYNCCDDDYFNKKDWYDFVENPDILRVYANLGERVRQ | |
| | ALLKTVQFCDAMRNAGIVGVLTLDNQDLNGNWYDFGDFIQTTPG | |
| | SGVPVVDSYYSLLMPILTLTRALTAESHVDTDLTKPYIKWDLLK | |
| | YDFTEERLKLFDRYFKYWDQTYHPNCVNCLDDRCILHCANFNVL | |
| | FSTVFPPTSFGPLVRKIFVDGVPFVVSTGYHFRELGVVHNQDVN | |
| | LHSSRLSFKELLVYAADPAMHAASGNLLLDKRTTCFSVAALTNN | |
| | VAFQTVKPGNFNKDFYDFAVSKGFFKEGSSVELKHFFFAQDGNA | |
| | AISDYDYYRYNLPTMCDIRQLLFVVEVVDKYFDCYDGGCINANQ | |
| | VIVNNLDKSAGFPFNKWGKARLYYDSMSYEDQDALFAYTKRNVI | |
| | PTITQMNLKYAISAKNRARTVAGVSICSTMTNRQFHQKLLKSIA | |
| | ATRGATVVIGTSKFYGGWHNMLKTVYSDVENPHLMGWDYPKCDR | |
| | AMPNMLRIMASLVLARKHTTCCSLSHRFYRLANECAQVLSEMVM | |
| | CGGSLYVKPGGTSSGDATTAYANSVFNICQAVTANVNALLSTDG | |
| | NKIADKYVRNLQHRLYECLYRNRDVDTDFVNEFYAYLRKHFSMM | |
| | ILSDDAVVCFNSTYASQGLVASIKNFKSVLYYQNNVFMSEAKCW | |
| | TETDLTKGPHEFCSQHTMLVKQGDDYVYLPYPDPSRILGAGCFV | |
| | DDIVKTDGTLMIERFVSLAIDAYPLTKHPNQEYADVFHLYLQYI | |
| | RKLHDELTGHMLDMYSVMLTNDNTSRYWEPEFYEAMYTPHTVLQ | |
| | AVGACVLCNSQTSLRCGACIRRPFLCCKCCYDHVISTSHKLVLS | |
| | VNPYVCNAPGCDVTDVTQLYLGGMSYYCKSHKPPISFPLCANGQ | |
| | VFGLYKNTCVGSDNVTDFNAIATCDWTNAGDYILANTCTERLKL | |
| | FAAETLKATEETFKLSYGIATVREVLSDRELHLSWEVGKPRPPL | |
| | NRNYVFTGYRVTKNSKVQIGEYTFEKGDYGDAVVYRGTTTYKLN | |
| | VGDYFVLTSHTVMPLSAPTLVPQEHYVRITGLYPTLNISDEFSS | |
| | NVANYQKVGMQKYSTLQGPPGTGKSHFAIGLALYYPSARIVYTA | |
| | CSHAAVDALCEKALKYLPIDKCSRIIPARARVECFDKFKVNSTL | |
| | EQYVFCTVNALPETTADIVVFDEISMATNYDLSVVNARLRAKHY | |
| | VYIGDPAQLPAPRTLLTKGTLEPEYFNSVCRLMKTIGPDMFLGT | |
| | CRRCPAEIVDTVSALVYDNKLKAHKDKSAQCFKMFYKGVITHDV | |
| | SSAINRPQIGVVREFLTRNPAWRKAVFISPYNSQNAVASKILGL | |
| | PTQTVDSSQGSEYDYVIFTQTTETAHSCNVNRFNVAITRAKVGI | |
| | LCIMSDRDLYDKLQFTSLEIPRRNVATLQAENVTGLFKDCSKVI | |
| | TGLHPTQAPTHLSVDTKFKTEGLCVDIPGIPKDMTYRRLISMMG | |
| | FKMNYQVNGYPNMFITREEAIRHVRAWIGEDVEGCHATREAVGT | |
| | NLPLQLGFSTGVNLVAVPTGYVDTPNNTDFSRVSAKPPPGDQFK | |
| | HLIPLMYKGLPWNVVRIKIVQMLSDTLKNLSDRVVFVLWAHGFE | |
| | LTSMKYFVKIGPERTCCLCDRRATCFSTASDTYACWHHSIGFDY | |
| | VYNPFMIDVQQWGFTGNLQSNHDLYCQVHGNAHVASCDAIMTRC | |
| | LAVHECFVKRVDWTIEYPIIGDELKINAACRKVQHMVVKAALLA | |
| | DKFPVLHDIGNPKAIKCVPQADVEWKFYDAQPCSDKAYKIEELF | |
| | YSYATHSDKFTDGVCLFWNCNVDRYPANSIVCRFDTRVLSNLNL | |
| | PGCDGGSLYVNKHAFHTPAFDKSAFVNLKQLPFFYYSDSPCESH | |
| | GKQVVSDIDYVPLKSATCITRCNLGGAVCRHHANEYRLYLDAYN | |
| | MMISAGFSLWVYKQFDTYNLWNTFTRLQSLENVAFNVVNKGHFD | |
| | GQQGEVPVSIINNTVYTKVDGVDVELFENKTTLPVNVAFELWAK | |
| | RNIKPVPEVKILNNLGVDIAANTVIWDYKRDAPAHISTIGVCSM | |
| | TDIAKKPTETICAPLTVFFDGRVDGQVDLFRNARNGVLITEGSV | |
| | KGLQPSVGPKQASLNGVTLIGEAVKTQFNYYKKVDGVVQQLPET | |
| | YFTQSRNLQEFKPRSQMEIDFLELAMDEFIERYKLEGYAFEHIV | |
| | YGDFSHSQLGGLHLLIGLAKRFKESPFELEDFIPMDSTVKNYFI | |
| | TDAQTGSSKCVCSVIDLLLDDFVEIIKSQDLSVVSKVVKVTIDY | |
| | TEISFMLWCKDGHVETFYPKLQSSQAWQPGVAMPNLYKMQRMLL | |
| | EKCDLQNYGDSATLPKGIMMNVAKYTQLCQYLNTLTLAVPYNMR | |
| | VIHFGAGSDKGVAPGTAVLRQWLPTGTLLVDSDLNDFVSDADST | |
| | LIGDCATVHTANKWDLIISDMYDPKTKNVTKENDSKEGFFTYIC | |
| | GFIQQKLALGGSVAIKITEHSWNADLYKLMGHFAWWTAFVTNVN | |
| | ASSSEAFLIGCNYLGKPREQIDGYVMHANYIFWRNTNPIQLSSY | |
| | SLFDMSKFPLKLRGTAVMSLKEGQINDMILSLLSKGRLIIRENN | |
| | RVVISSDVLVNN | |
| ORF3a | MDLFMRIFTIGTVTLKQGEIKDATPSDFVRATATIPIQASLPFG | 285 |
| | WLIVGVALLAVFQSASKIITLKKRWQLALSKGVHFVCNLLLLFV | |
| | TVYSHLLLVAAGLEAPFLYLYALVYFLQSINFVRIIMRLWLCWK | |
| | CRSKNPLLYDANYFLCWHTNCYDYCIPYNSVTSSIVITSGDGTT | |
| | SPISEHDYQIGGYTEKWESGVKDCVVLHSYFTSDYYQLYSTQLS | |
| | TDTGVEHVTFFIYNKIVDEPEEHVQIHTIDGSSGVVNPVMEPIY | |
| | DEPTTTTSVPL | |
| ORF6 | MFHLVDFQVTIAEILLIIMRTFKVSIWNLDYIINLIIKNLSKSL | 286 |
| | TENKYSQLDEEQPMEID | |
| ORF7a | MKIILFLALITLATCELYHYQECVRGTTVLLKEPCSSGTYEGNS | 287 |
| | PFHPLADNKFALTCFSTQFAFACPDGVKHVYQLRARSVSPKLFI | |
| | RQEEVQELYSPIFLIVAAIVFITLCFTLKRKTE | |
| ORF8 | MKFLVFLGIITTVAAFHQECSLQSCTQHQPYVVDDPCPIHFYSK | 288 |
| | WYIRVGARKSAPLIELCVDEAGSKSPIQYIDIGNYTVSCLPFTI | |
| | NCQEPKLGSLVVRCSFYEDFLEYHDVRVVLDFI | |
| ORF10 | MGYINVFAFPFTIYSLLLCRMNSRNYIAQVDVVNFNLT | 289 |

In certain examples herein, the primers (or ID-Oligos) described herein were identified and designed based on the SARS-CoV-Urbani virus (GenBank Accession No. AY278741.1 (SEQ ID NO: 2), which is hereby incorporated by reference in its entirety), but it will be readily understood that embodiments described herein are not limited to this isolate and encompass detection of other isolates and variants of other coronaviruses. Table 5 identifies suitable coronavirus sequences for use in accordance with embodiments of the present disclosure (see also sequence information in Table 9).

TABLE 5
Exemplary Coronavirus Genome Sequences

| | GenBank | | |
|---|---|---|---|
| | Accession | SEQ | |
| Coronavirus Name | No.* | ID No. | Description |
| Severe acute respiratory syndrome | NC_045512.2 | 1 | Complete genome; 29,903 bp |
| coronavirus 2 isolate Wuhan-Hu-1 | | | ss-RNA (18-JUL.-2020) |
| SARS coronavirus Urbani | AY278741.1 | 2 | Complete genome; 29,727 bp |
| | | | RNA (25-JUL.-2016) |
| Betacoronavirus England 1 isolate | NC_038294.1 | 3 | Complete genome; 30,111 bp |
| H123990006 | | | RNA linear (22-NOV.-2020) |
| Middle East Respiratory | KX154693.1 | 4 | Complete genome; 30,096 bp |
| Syndrome Coronavirus Isolate | | | RNA linear (30-APR.-2017) |
| Hu/Riyadh-KSA-13127/2016 | | | |
| Severe acute respiratory syndrome | MT263402.1 | 5 | Complete genome; 29,844 bp |
| coronavirus 2 isolate SARS-CoV- | | | RNA (01-OCT.-2020) |
| 2/human/USA/WA-UW322/2020 | | | |
| Human coronavirus HKU1, | NC_006577.2 | 6 | Complete genome; 29,926 bp |
| complete genome | | | ss-RNA (20-NOV.-2020) |
| Human coronavirus OC43 strain | MN306036.1 | 7 | Complete genome; 30,681 bp |
| HCoV_OC43/Seattle/USA/SC0682/ | | | RNA (21-FEB.-2020) |
| 2019 | | | |
| Human coronavirus NL63 strain | MN306018.1 | 8 | Complete genome; 27,484 bp |
| HCoV_NL63/Seattle/USA/SCO179/ | | | RNA (21-FEB.-2020) |
| 2018 | | | |
| Human coronavirus 229E strain | MN369046.1 | 9 | Complete genome; 27,075 bp |
| HCoV_229E/Seattle/USA/SC9724/ | | | RNA (21-FEB.-2020) |
| 2018 | | | |

*Each of which is hereby incorporated by reference in its entirety.

As of January 2021, there were more than 414,575 complete genomic sequences of SARS-CoV-2 that have been shared globally through publicly accessible databases (see Pan American Health Organization/World Health Organization. Occurrence of variants of SARS-CoV-2 in the Americas. 26 Jan. 2021, which is hereby incorporated by reference in its entirety) and the methods disclosed herein are suitable for detecting the presence of each of these genomic sequences in a sample. In some embodiments, the target viral genome is a variant SARS-CoV-2 genome, as compared to a reference sequence (e.g., the reference sequence of SEQ ID NO: 1). In some embodiments, the primers (or ID-Oligos) described herein may be directed to a portion of a variant SARS-CoV-2 target viral genome corresponding to the portions of SEQ ID NO:1 identified herein above as encoding a structural protein such as surface protein (e.g., corresponding to nucleic acid residues 21,563-25,384 of SEQ ID NO:1), envelope protein (e.g., corresponding to nucleic acid residues 26,245-26,472 of SEQ ID NO:1), membrane protein (e.g., corresponding to nucleic acid residues 26,523-27,191 of SEQ ID NO:1), and nucleocapsid protein (e.g., corresponding to nucleic acid residues 28,274-29,533 of SEQ ID NO:1), and/or one or more of the six accessory proteins, encoded by ORF3a (e.g., corresponding to nucleic acid residues 25,393-26,220 of SEQ ID NO:1), ORF6 (e.g., corresponding to nucleic acid residues 27,202-27,387 of SEQ ID NO:1), ORF7a (e.g., corresponding to nucleic acid residues 27,394-27,759 of SEQ ID NO:1), ORF7b (e.g., corresponding to nucleic acid residues 27,756-27,887 of SEQ ID NO:1), and ORF8 regions (e.g., corresponding to nucleic acid residues 27,894-28,259 of SEQ ID NO:1).

Exemplary SARS-CoV-2 variants are identified in Table 6 below (see also sequence information in Table 9) together with characteristic mutations. As described herein, one or more ID-Oligos may be designed to be complementary to variant-specific regions of the target viral genome and, in certain embodiments, are complementary (or hybridize) to the region of the target viral genome corresponding to (or encoding) the mutations identified in Table 6 below. Such variant-specific regions corresponding to, e.g., the characteristic mutations identified in Table 6 may be determined by examining the corresponding (or encoding) portions of the exemplary accession or GISAID sequences identified in Table 6 or by comparing the characteristic mutation with the corresponding position(s) of the reference sequence (i.e., unmutated, e.g., SEQ ID NO:1 and the corresponding encoded amino acid sequences shown in Table 4) to determine variant-specific sequences that would result in the mutated amino acid identified.

TABLE 6
SARS-CoV-2 Variants*

| Variant Designation | Characteristic Mutations (Protein: Mutation)** |
|---|---|
| B.1.1.7 | ORF1ab: T1001I, A1708D, I2230T, Δ3675-3677 |
| (20I/501Y.V1) | SGF |
| (see, e.g., GenBank Accession Nos. | Spike (S): Δ69-70 HV, A144 Y, N501Y, A570D, |
| MW735442.1 (SEQ ID NO: 290), | D614G, P681H, T761I, S982A, D1118H |
| MW692118.1 (SEQ ID NO: 291), | ORF8: Q27stop, R52I, Y73C |
| MW531680.1 (SEQ ID NO: 292)***) | Nucleocapsid (N): D3L, S235F |
| B.1.351 (20H/501Y.V2) | ORF1ab: K1655N |
| (see, e.g., GISAID number | Envelope (E): P71L |
| EPL_ISL_6785974***) | Nucleocapsid (N): T205I |
| | Spike (S): K417N, E484K, N501Y, D614G, A701V |
| P.1 | ORF1ab: F681L, I760T, S1188L, K1795Q, Δ3675- |
| (20J/501Y.V3)(see, e.g., GenBank | 3677 SGF, E5662D |
| Accession No. MW642250 (SEQ ID | Spike (S): L18F, T20N, P26S, D138Y, R190S, |
| NO: 293)***) | K417T, E484K, N501Y, D614G, H655Y, T1027I |
| | ORF3a: C174G |
| | ORF8: E92K |
| | ORF9: Q77E |
| | ORF14: V49L |
| | Nucleocapsid (N): P80R |
| B.1.526 (see, e.g., GISAID number | Spike: (L5F*), T95I, D253G, (S477N*), (E484K*), |
| EPL_ISL_962493***) | D614G, (A701V*) |
| | ORF1a: L3201P, T265I, Δ3675/3677 |
| | ORF1b: P314L, Q1011H |
| | ORF3a: P42L, Q57H |
| | ORF8: T11I, |
| | 5′UTR: R81C |
| P.2 | Spike: E484K, D614G, V1176F |
| | ORF1a: L3468V, L3930F |
| | ORF1b: P314L |
| | N: A119S, R203K, G204R, M234I, |
| | 5′UTR: R81C |
| B.1.427 | Spike: L452R, D614G |
| B.1.429 (see, e.g., GISAID number | Spike: S13I, W152C, L452R, D614G |
| EPI_ISL_847590***) | |

*See, e.g. Centers for Disease Control and Prevention, Galloway et al., “Emergence of SARS-CoV-2 B.1.1.7 Lineage - United States, Dec. 29, 2020 - Jan. 12, 2021,” Morbidity and Mortality Report 70(3): 95-99 (2021), which is hereby incorporated by reference in its entirety). **CDC, Scientific Brief on Emerging Variants. Each sequence identified is hereby incorporated by reference in its entirety. *** Each GenBank Accession No. or GISAID reference no. is hereby incorporated by reference in its entirety (see also Table 9).

In some embodiments, the target viral genome is from Zika virus. Zika infection in humans was first identified in Uganda and the United Republic of Tanzania in 1952, and human Zika infections have been sporadically detected in Africa and Asia for >50 years since the initial isolation (Kawai et al., “Increased Growth Ability and Pathogenicity of American and Pacific-Subtype Zika Virus (ZIKV) Strains Compared with a Southeast Asian-Subtype ZIKV Strain,” PLoS 13(6):e0007387 (2019), which is hereby incorporated by reference in its entirety). The clinical symptoms caused by Zika infection are generally self-limiting and include fever, rash, headache, joint and muscle pain, and conjunctivitis. Approximately 60-70% and 90% of symptomatic Zika-infected patients develop fever and rash, respectively.

The Zika genome is comprised of a single-stranded, positive-sense RNA that encodes three structural proteins (C, prM, and E) and seven non-structural proteins (NS1, NS2A, NS2B, NS3, NS4A, NS4B, and NS5) in one open reading frame. Zika is classified into two lineages, African and Asian/American. Phylogenetic analyses performed using complete Zika genomes indicate that ZIKV strains in the Asian/American lineage can be divided into three subtypes, American, Pacific, and Southeast Asian, which present several differences in their amino acid sequences. In some embodiments, the at least partially single-stranded viral genome is a Zika virus genome having one of the sequences identified in Table 7 below (see also sequence information in Table 9).

TABLE 7
Exemplary Zika Genome Sequences

| | GenBank | SEQ ID | |
|---|---|---|---|
| Zika Virus Name | Accession No.* | No. | Description |
| Zika virus isolate 1034511 | KX262887.1 | 296 | Complete genome; 10,806 bp |
| | | | RNA (20-MAY 2016) |
| Zika virus | AY632535.2 | 297 | Complete genome; 10,794 bp |
| | | | RNA (01-AUG.-2019) |
| Zika virus strain H/PF/2013 | KJ776791.2 | 298 | Complete genome; 10,807 bp |
| | | | RNA (31-AUG.-2016) |
| Zika virus strain PRVABC- | KX377337.1 | 299 | Complete genome; 10,807 bp |
| 59 | | | RNA (22-JUN.-2016) |
| Zika virus isolate | KY766069.1 | 294 | Complete genome; 11,155 bp |
| Pf13/251013-18 | | | ss-RNA(14-DEC.-2017) |
| Zika virus isolate ZIKV/H. | NC_035889.1/ | 295 | Complete genome; 10,808 bp |
| sapiens/Brazil/Natal/2015 | KU527068.1 | | RNA (24-MAY 2019) |

*Each of which is hereby incorporated by reference in its entirety.

In some embodiments, the target viral genome is from a Hepatitis C virus. Hepatitis C virus (HCV) infection afflicts more than 170 million people worldwide, with the great majority of patients with acute hepatitis C developing chronic HCV infection. It can ultimately result in liver cirrhosis, hepatic failure or hepatocellular carcinoma, which are responsible for hundreds of thousands of deaths each year. HCV has a narrow host specificity and tissue tropism. HCV is transmitted exclusively through direct blood-to-blood contacts between humans.

In some embodiments, the target viral genome is a Hepatitis C virus genome having one of the sequences identified in Table 8 below (see also sequence information in Table 9).

TABLE 8
Exemplary HCV Genome Sequences

| | GenBank | SEQ ID | |
|---|---|---|---|
| Coronavirus Name | Accession No.* | No. | Description |
| Hepatitis C virus | AB047639.1 | 300 | Hepatitis C virus (isolate |
| strain JFH-1 | (polyprotein | | JFH-1) genomic RNA, |
| | BAB32872.1) | | complete genome |
| Hepatitis C virus | NC_004102.1 | 301 | Complete genome; |
| genotype 1 | | | 9,646 bp ss-RNA |
| | | | (11 JUL. 2019) |
| Hepatitis C virus | NC_030791.1 | 302 | Complete genome; |
| genotype 7 | | | 9,443 bp RNA |
| | | | (28 MAY 2019) |

*Each of which is hereby incorporated by reference in its entirety.

A single-stranded template region to the selected portions of the target to which the primer (or ID-Oligo) is complementary (or hybridizes) may be any portion of the target that is of interest for detection according to the methods described herein. In some embodiments, the methods described herein are carried out using one or more oligonucleotide molecules complementary to different portions of the target viral genome (i.e., one or more ID-Oligos, each targeting a different portion of the target viral genome).

In some embodiments, the one or more ID-Oligos are designed to target portions of the target viral genome that are strain-specific, variant-specific, or/or conserved across all target viral genome strains and variants. As used herein, an ID-Oligo is said to be “specific” for the target viral genome if it can be used in the methods described herein under conditions that permit it to detect the target viral genome without exhibiting cross-reactivity to a single-stranded nucleic acid sequence that is not from the target viral genome (e.g., a non-viral nucleic acid or a nucleic acid from another virus). For example, the one or more ID-Oligos may be designed to target portions of the SARS-CoV-2 genome that are SARS-CoV-specific, SARS-CoV-2 strain-specific (e.g., specific to SARS-CoV-2), SARS-CoV-2 variant-specific, or conserved across all SARS-CoV-2 strains and variants. In accordance with such embodiments, the one or more SARS-CoV-specific ID-Oligos do not cross-react with other at least partially single stranded virus genomes (e.g., Hepatitis C Virus, Zika Virus, Influenza Virus, etc.).

In some embodiments, the methods described herein are carried out to perform a general screen for the presence of one or more target viral genomes. In accordance with such embodiments, the method includes a population of ID-Oligos (or primers) comprising subpopulations of ID-Oligos, where each sub-population of ID-Oligos is designed to be complementary to a different target viral genome. In one example, a general screen for influenza viruses may be carried out using multiple ID-Oligos (e.g., an array) that would be robust to the rapid evolution of influenza virus strains, due to mutation and reassortment. Similarly, a general screen for HIV could involve multiple ID-Oligos that are robust to the rapid evolution by point mutations and natural selection. In contrast, a number of highly specific ID-Oligos could be chosen to detect a specific strain of influenza that might help pinpoint vaccine priorities or might help identify emergence of drug resistance in strains of HIV.

In some embodiments, the methods described herein are carried out to perform a general screen for the presence of one or more target viral genomes. In accordance with such embodiments, the method includes providing a population of ID-Oligos comprising subpopulations of ID-Oligos, where each sub-population of ID-Oligos is designed to be complementary to a different portion of one or more target viral genomes.

Oligonucleotides that are the reverse complement of selected portions of the target are referred to herein as oligonucleotide primers, ID-oligonucleotides, ID-Oligos, or primers. Primers described herein refer to a nucleic acid molecule that hybridizes in a sequence specific manner to a complementary nucleic acid molecule, whereby the primer is capable of extension from its 3′-end through incorporation of dNTPs by a polymerase. The primers of embodiments of the disclosure comprise a nucleotide sequence that is complementary or substantially complementary to a selected portion of a target sequence. The terms “complementary” and “substantially complementary” refer to base pairing between nucleotides such as, for instance, between an oligonucleotide primer and the target or selected portion of the target. Complementary nucleotides are, generally, adenine and thymine, adenine and uracil, and guanine and cytosine. Primers do not require complete complementarity in order to hybridize to their target nucleotide sequence. Primer sequences of the disclosure may be modified to some extent without loss of utility as specific primers. In some embodiments, the primer(s) disclosed herein are at least 80%, at least 85%, at least 90%, at least 91%, at least 92%, at least 93%, at least 94%, at least 95%, at least 96%, at least 97%, at least 98%, at least 99% or 100% complementary to the target sequence or selected portion thereof.

The oligonucleotide molecule complementary to a selected portion of the target viral genome may be designed by first identifying a target single-stranded region within the viral genome. In some embodiments, the target viral genome is analyzed for areas of secondary structure that might inhibit binding of a complementary oligonucleotide molecule. Accordingly, in some embodiments, the target-single-stranded region within the viral genome does not form secondary-structures that inhibit hybridization of a complementary oligonucleotide. Next, the target at least partially single-stranded viral genome immediately 3′ or 5′ to the identified target single-stranded region (depending whether the target virus has a positive or negative sense genome) is identified and the reverse complement sequences of the virus-specific regions are synthesized as ID-Oligos. Depending on the objectives, ID-Oligos can be designed to hybridize with regions conserved within a family or genus of viruses (e.g., to identify any betacoronavirus, if that is the target) or to one or more regions specific to an individual species of virus (e.g., SARS-CoV), or to one or more regions specific to an individual strain of virus (e.g., SARS-CoV-2), or to one or more regions specific to an individual variant of a strain of virus (e.g., SARS-CoV-2 variant B.1.1.7, SARS-CoV-2 variant B.1.351, SARS-CoV-2 variant P.1, SARS-CoV-2 variant B.1427, SARS-CoV-2 variant B.1.429, SARS-CoV-2 variant B.1.526, SARS-CoV-2 variant B.1.525, and/or SARS-CoV-2 variant P.2). One or more sequences may be selected for use as an ID-Oligo that will hybridize to the target viral genome (e.g., a target at least partially single-stranded RNA genome or a target at least partially single-stranded DNA genome).

In some embodiments, multiple primers (ID-Oligos) are provided for hybridizing to different complementary portions of one or more target partially single-stranded viral genomes. The multiple portions of the one or more target single-stranded viral genome(s) may be adjacent to regions that are uracil/thymine-free. In some embodiments, all of the different single-stranded template regions are uracil/thymine-free regions. A contemplated method in accordance with such an embodiment is a multiplex assay in which a plurality of ID-Oligos or “primers” are utilized to determine the presence or absence of one or more of a plurality of predetermined target single-stranded viral genome sequences (single-stranded template regions) in a sample. A particularly useful area for such multiplex assays is in screening assays.

In a multiplexed embodiment of the above method, the sample is admixed with a plurality of different ID-Oligos or “primers”. In this embodiment, the analytical output for a certain result with one of the capture oligonucleotides is distinguishable from the analytical output from the opposite result with all of the primers.

In accordance with this embodiment, for example, a solid support may contain multiple primers specific for multiple target nucleic acids. Each primer can be localized at defined positions or regions of the solid support, or synthesized on the surface at defined positions or regions of the solid support in situ. Such support facilitates parallel analysis of multiple primer-bound target nucleic acids. Such supports are also appropriate for high throughput screening.

In some embodiments, the ID-Oligos are analyzed for self-dimerization and/or cross-dimerization. In some embodiments, the ID-Oligos do not self-dimerize and/or cross-dimerize (e.g., with each other). In some embodiments, the guanine/cytosine (G/C) content (as a percent of the total bases of the primer) of the primer sequence is greater than 35%, 40%, 45%, 50%, 55%, or 60% to prevent hairpin formation of the primer. The primer melting temperature (Tm) is the temperature at which one half of the primer-target duplex will dissociate to become single stranded and indicates the duplex stability. In some embodiments, the primer has a melting temperature in the range of 50-65, 50-60, 55-65, or 55-60 degrees Celsius.

The oligonucleotide primers can be in the form of ribonucleotides, deoxyribonucleotides, modified ribonucleotides, modified deoxyribonucleotides, modified phosphate-sugar-backbone oligonucleotides, nucleotide analogs, and mixtures thereof. In some embodiments, the oligonucleotide primers are single-stranded deoxyribonucleic acid (DNA) molecules. The primers utilized in accordance with any embodiment of the disclosure may be prepared using any suitable method, such as conventional phosphotriester and phosphodiester methods or automated embodiments thereof so long as the primers are capable of hybridizing to their target nucleotide sequences of interest. The primers utilized in the methods described herein to detect the presence or absence of the target sequence are each at least 6 nucleotides in length. In some embodiments, the ID-Oligonucleotide is (or are at least) 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. Where more than one primer is used, each primer may be (or at least), independently, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. Likewise, where an ID-Oligo (or primer) described herein has a nucleotide sequence that is the reverse complement (or hybridizes) to a portion of a genomic sequence, in some embodiments, the portion is (or is at least) 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, the primer has the sequence of one of SEQ ID NOs:90-169, SEQ ID NOs:251-269, and SEQ ID NOs:273-275, or a fragment thereof at least 6 nucleotides in length. In certain embodiments, the primer has the nucleotide sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleotide sequence of one of SEQ ID NOs: 90-169, SEQ ID NOs:251-269, and SEQ ID NOs:273-275, or a fragment thereof at least 6 nucleotides in length.

In some embodiments, the ID-Oligo (or primer) has a nucleotide sequence of at least 6 nucleotides in length corresponding to the reverse complement of the SARS-CoV-2 genome of SEQ ID NO:1, which is shown in Table 9 as SEQ ID NO:250.

In some embodiments, the ID-Oligo (or primer) has a nucleotide sequence that is the reverse complement to a portion of a sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of the target viral genome sequences identified herein. In some embodiments, the ID-Oligo (or primer) has a nucleotide sequence that is the reverse complement to a portion of a genomic sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of SEQ ID NOs:1-9, as identified in Table 5 above.

In some embodiments, the primer has a nucleotide sequence that is the reverse complement to a portion of the SARS-CoV-2 reference sequence (i.e., SEQ ID NO:1, Table 5). In some embodiments, the primer has a nucleotide sequence that is the reverse complement to a portion of a variant genomic sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to the SARS-CoV-2 reference sequence (i.e., SEQ ID NO:1, Table 5). In some embodiments, the primer described herein is the reverse complement of a portion of the SARS-CoV-2 genomic sequence encoding the surface protein (i.e., nucleic acid residues 21,563-25,384 of SEQ ID NO:1), the envelope protein (i.e., nucleic acid residues 26,245-26,472 of SEQ ID NO:1), the membrane protein (i.e., nucleic acid residues 26,523-27,191 of SEQ ID NO:1), the nucleocapsid protein (i.e., nucleic acid residues 28,274-29,533 of SEQ ID NO:1), and/or the accessory proteins, encoded by ORF3a (i.e., nucleic acid residues 25,393-26,220 of SEQ ID NO:1), ORF6 (i.e., nucleic acid residues 27,202-27,387 of SEQ ID NO:1), ORF7a (i.e., nucleic acid residues 27,394-27,759 of SEQ ID NO:1), ORF7b (i.e., nucleic acid residues 27,756-27,887 of SEQ ID NO:1), and ORF8 regions (i.e., nucleic acid residues 27,894-28,259 of SEQ ID NO:1). See SEQ ID NO: 1, Table 5. In some embodiments, the ID-Oligo (or primer) has a nucleotide sequence that is the reverse complement to a portion of a genomic sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of SEQ ID NOs:10-89 and/or 270-272.

As used herein, “luciferase” refers to an oxygenase that catalyzes a luminescence reaction as follows:

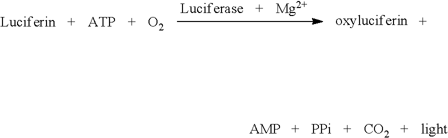

Thus, luciferase refers to an enzyme or photoprotein that catalyzes a bioluminescent reaction (a reaction that produces bioluminescence) and is a naturally occurring, recombinant, or mutated luciferase unless otherwise specified. When present in nature, luciferase can be readily obtained from an organism by one of ordinary skill in the art. If the luciferase is a naturally occurring luciferase, or a recombinant or mutant luciferase (e.g., a luciferase that retains activity in a luciferase-luciferin reaction of a naturally occurring luciferase), the nucleic acid encoding the luciferase is expressed. It can be easily obtained from transformed cultures of bacteria, yeast, mammalian cells, insect cells, plant cells and the like. Furthermore, recombinant or mutant luciferases can be easily obtained from in vitro cell-free systems that use nucleic acids encoding luciferases. Luciferase is commercially available from, e.g., Promega Corporation (Madison, Wis.). Luciferases, modified mutants or variants thereof are also known in the art and described in, for example, Thorne et al, “Illuminating Insights into Firefly Luciferase and Other Bioluminescent Reporters Used in Chemical Biology,” Chemistry & Biology 17(6):646-657 (2010), which is hereby incorporated by reference in its entirety.

The “polymerase extension” reaction according to the application includes all forms of template-directed polymerase catalyzed nucleic acid synthesis reactions. As described above, methods herein involve subjecting a polymerase extension mixture to conditions under which a hybridization product from a double-stranded nucleic acid start portion is extended by addition of nucleotides complementary to the single-stranded template region, which is a polymerase extension reaction. Conditions and reagents for primer extension reactions are known in the art, and any of the standard methods, reagents and enzymes, etc. can be used at this stage (see, for example, Green & Sambrook, Molecular Cloning: A Laboratory Manual (4^{th} ed. Cold Spring Harbor Laboratory Press, 2012) which is hereby incorporated by reference in its entirety). Thus, the extension reaction in its most basic form is performed in the presence of the primer (or ID-oligo), deoxynucleotides (dNTP), and a suitable polymerase enzyme, which depends on whether the target is DNA or RNA.

Polymerases suitable for use in the methods of the present application are well known in the art. In some embodiments, the target at least partially single-stranded viral genome comprises DNA. The polymerase may be a DNA polymerase, e.g., full length BST DNA polymerase, large fragment BST DNA polymerase, BST 2.0 DNA Polymerase, Klenow fragment (3′ to 5′ exo), and DNA Polymerase I (large Klenow fragment). In some embodiments, the target at least partially single-stranded viral genome comprises RNA. Suitable reverse transcriptase enzymes for use in accordance with the present disclosure include for example ProtoScript® II RT, M-MuLV RT, and AMV RT. In some embodiments, the polymerase is stable at temperature ranges from about 15 to about 100° C. The conditions can be selected according to the choice, according to the procedures known in the art.

Polymerase extension techniques for use in the methods of the present application are isothermal techniques (i.e., those that are performed at a single temperature or where the major aspect of the amplification process is performed at a single temperature). Such techniques rely on the ability of a polymerase to copy the template strand being extended to form a bound duplex.

Briefly, as used in the methods of the present application, the polymerase extension occurs when the polymerase (e.g., DNA polymerase or reverse transcriptase) binds to a primer-target hybrid (i.e., double-stranded DNA, which is a hybridization product comprising a double-stranded nucleic acid start portion) and extends the complementary DNA strand based on the single-stranded oligonucleotide sequence adjacent to the primer-target hybrid. The extension reaction occurs using available nucleotides provided in a deoxynucleotide (dNTP) mixture added to the reaction.

Deoxynucleotides (dNTPs) include deoxy-adenosine triphosphate (dATP), deoxy-thymidine triphosphate (dTTP), deoxy-cytosine triphosphate (dCTP), and deoxy-guanosine triphosphate (dGTP). An unmodified form of dATP, if present in the polymerase extension mixture or other dNTP mixtures described herein, may bind to and be hydrolyzed by, luciferase. This reaction of luciferase with the dATP provided with the dNTP mixture may cause unwanted decay high in luminescent signal background, as well unwanted rapid hydrolysis of other reagents including luciferin and magnesium. Accordingly, in some embodiments, unmodified deoxyadenosine triphosphate (dATP) is excluded from the polymerase extension mixture (and other dNTP mixtures described herein). In some embodiments, the polymerase extension mixture (and other dNTP mixtures described herein) is substantially free of unmodified deoxyadenosine triphosphate (dATP). In some embodiments, the polymerase extension mixture (and other dNTP mixtures described herein) comprises a modified form of dATP that does not react with luciferase or does not react with luciferase to cause significant increased background in luminescent signal. In some embodiments, the polymerase extension mixture (and other dNTP mixtures described herein) comprises an alpha phosphate-modified form of dATP. In some embodiments, the polymerase extension mixture (and other dNTP mixtures described herein) comprises dATPaphaS (dATPαS), having the following structural formula:

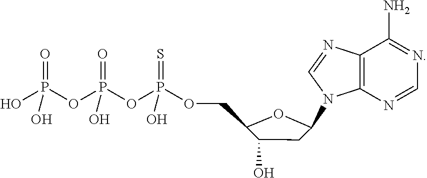 [SMILES: Nc1ncnc2c1ncn2[C@H]1C[C@H](O)[C@@H](COP(O)(=S)OP(=O)(O)OP(=O)(O)O)O1]

In some embodiments, the single-stranded template region of the target sequence is free of thymine (in the case of a target DNA sequence) or uracil (in the case of a target RNA sequence). In this case, the primer (ID-oligo) is designed to hybridize to a selected portion of the target that is adjacent to a single-stranded template region of the target sequence that is free of thymine (in the case of a target DNA sequence) or uracil (in the case of a target RNA sequence). The single-stranded template region of the target may be at least 5, 10, 15, 20, 25, 30, 25, 40, 45, 50, 55, 60, or more nucleotides in length. As would be readily appreciated, in such embodiments, the polymerase extension reaction by addition of complementary nucleotides to single-stranded template region would not require dATP. Accordingly, in some embodiments, the polymerase extension mixture (and other dNTP mixtures described herein) may be free of modified and unmodified dATP.

As described above, polymerase extension techniques for use in the methods of the present application are isothermal techniques (i.e., those that are performed at a single temperature or where the major aspect of the amplification process is performed at a single temperature). Accordingly, in certain embodiments, the polymerase extension reaction is carried out at a temperature of 0, 5, 10, 15, 20, 25, 30, 35, 40, 45, 50, 55, 60, 65, 70, 75, 80, 85, 90, or 95 to 100° C. In some embodiments, the polymerase extension reaction is carried out at a temperature of 15 to 45° C. In some embodiments, the polymerase extension reaction is carried out at a temperature of 25 to 40° C. In some embodiments, the polymerase extension reaction is carried out at a temperature of 25 to 45° C.

The polymerase extension reaction releases two phosphate groups (PPi) per nucleotide added to the nucleic acid strand. In the methods of the present application, the release of these free phosphates (PPi) can then be used to facilitate detection of the target nucleic acid molecule in the sample. Specifically, through the conversion of PPi to ATP via enzymatic reaction and the subsequent bioluminometric detection of ATP using the signal-transducing molecule luciferase, the presence or absence of a target nucleic acid molecule can be detected.

Luciferase and luciferin are used, in combination, to identify the target nucleic acid since the amount of light generated is substantially proportional to the amount of ATP generated, and, in turn, is directly proportional to the amount of nucleotide incorporated and target nucleic acid present. Thus, the method also includes providing luciferin and O₂, where the luciferin and O₂ are added to the reaction.

As described above, the method described herein involves subjecting a polymerase extension mixture to conditions under which the target nucleic acid molecule is extended and releases free phosphates. Adenosine triphosphates (ATP) are then produced from the released free phosphates via an enzymatic reaction, which is then metabolized with a luciferase to produce the bioluminescent readout signal. In accordance with this, in one embodiment, producing adenosine triphosphates comprises subjecting the released free phosphates to a coupled glyceraldehyde 3-phosphate dehydrogenase-phosphoglycerate kinase (GAPDH-PGK) enzymatic reaction to produce adenosine triphosphate.

In this embodiment, the enzymatic reaction involves the GAPDH reacting with PGK in the presence of PPi, adenosine diphosphate (ADP), nicotinamide adenine dinucleotide (NAD+), and glyceraldehyde 3-phosphate (GAP) to produce ATP. ATP then reacts with luciferase to produce a measurable signal. The reaction scheme is shown below:

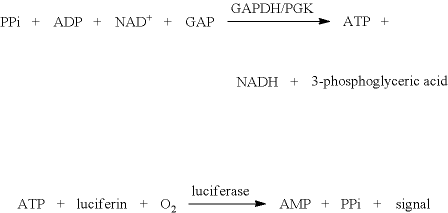

In another embodiment, adenosine triphosphates may be produced by contacting the released free phosphates with adenosine 5′-phosphosulfate (APS) in the presence of adenosine triphosphate sulfurylase (ATP-sul) to produce adenosine triphosphate. ATP then reacts with luciferase to produce a measurable signal. The reaction scheme is shown below:

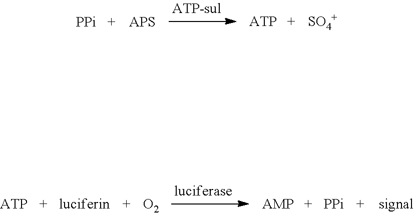

The amount of light produced can be easily determined using a suitable reader, e.g., a sensitive device in the appropriate wavelength such as a luminometer. Examples of luminometers include, but are not limited to, SpectraMax L, GloMax® 96-well microplate luminometer, GloMax®-20/20 single-tube luminometer, GloMax® Multi- with Instinct™ software, GloMax®-Multi Jr single tube multimode reader, LUMIstar OPTIMA, LEADER HC′ luminometer, LEADER 450i luminometer, and LEADER 50i luminometer. Thus, luminometric methods offer the advantage of being capable of quantitation.

In one embodiment, the bioluminescent readout signal is quantified to determine the presence and/or concentration of the target nucleic acid molecule in the sample. The amount of target nucleic acid can be determined from the peak amplitude of the luminescent signal, and/or the time it takes the signal to reach its peak amplitude, and/or the integrated amount of signal emitted over a period of time, and/or from the rate in which luminescence is produced.

In one embodiment, the target nucleic acid molecule is present in the sample in a concentration of less than 10⁻⁵ moles per liter.

In one embodiment, the target nucleic acid molecule or fragment thereof has been amplified or subjected to an amplification process. Amplification techniques are well known in the art. Any suitable RNA or DNA amplification technique may be used. In certain example embodiments, the RNA or DNA amplification is an isothermal amplification (e.g., nucleic acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR (including all PCR-based amplification techniques), multiple displacement amplification (MDA), rolling circle amplification (RCA), ligase chain reaction (LCR), or ramification amplification method (RAM). In such embodiments, the target may be (or the sample containing the target may include) an amplification product. In some embodiments, the target is not amplified (using, e.g., PCR) and the target is not (or the sample containing the target does not contain or is substantially free of) an amplification product.

In one embodiment, the presence of the target nucleic acid molecule in the sample is determined by a procedure comprising calculating an initial rate of bioluminescent signal production, calculating what time period is needed to achieve peak bioluminescence, and calculating bioluminescent signal peak amplitude or integrated bioluminescent signal from time zero to peak bioluminescence.

These procedures can be used individually or as part of an analytical method that incorporates two or more procedures for better quantitation. For each procedure or combination of procedures, threshold values can be pre-determined and calibrated to known amounts of nucleic acid targets. Predetermined values can also be useful in identifying similar but non-identical sequences (i.e., mutations) to the desired target. Additionally, the values provided by these analytical procedures can be evaluated against a cut-off value to provide a present/absent measurement, or as a scale to enable quantitative readout.

In accordance with any of the embodiments described herein, the enzymes may be coupled to a support (e.g., a gel, matrix, nanoparticle, a micro-titer plate well, a membrane, a filter, a microchip, sepharose, thiopropyl-sepharose, sephadex, agarose, silica, magnetic beads, methacrylate beads, or other solid or part-solid (e.g., gel) support).

In certain embodiments, the reactions of the present application are performed in solution. Accordingly one or more enzymes according to embodiments described herein may remain in solution. The term “in solution” refers to any assay in which the target nucleic acid is detected while in solution or in suspension. The term “in solution” may also refer to any assay in which one or more enzymes of the reaction are in solution or suspension. For example, a reaction according to the present application may be performed with a target at least partially single-stranded viral genome that is in solution and/or an enzyme (or enzymes) that are in solution. In some embodiments, where multiple target at least partially single-stranded viral genomes or in which multiple portions of the target at least partially single-stranded viral genome are detected, a first hybridization to a target single-stranded viral genome (or portion thereof) can be performed with a first ID-Oligo, and a second hybridization to the target nucleic acid can be performed with a second ID-Oligo. Such multiple hybridizations can include a washing step to remove any undesirable (e.g., non-hybridizing sequences) components.

In some embodiments, the reactions of the present application are carried out with one or more target at least partially single-stranded viral genome(s) and/or one or more enzymes, where the one or more target at least partially single-stranded viral genome(s) and/or the one or more enzymes are coupled to a solid support (e.g., a gel, matrix, nanoparticle, a micro-titer plate well, a membrane, a filter, a microchip, sepharose, thiopropyl-sepharose, sephadex, agarose, silica, magnetic beads, polystyrene, methacrylate beads, or other solid or part-solid (e.g., gel) support). For example, one or more enzymes according to embodiments described herein may be tethered to a nanoparticle (see, e.g., FIG. 4 and FIG. 5). In another example, one or more target single-stranded viral genomes (or portions thereof) are tethered to a solid support (e.g., a gel, matrix, nanoparticle, a micro-titer plate well, a membrane, a filter, a microchip, sepharose, thiopropyl-sepharose, sephadex, agarose, silica, magnetic beads, polystyrene, methacrylate beads, or other solid or part-solid (gel) support). The one or more target single-stranded viral genomes (or portions thereof) may be tethered to a solid support in the form of an array.

One or more enzymes according to embodiments of the present application may be coupled to a solid support. For example, in accordance with the above embodiments, the glyceraldehyde 3-phosphate dehydrogenase, the phosphoglycerate kinase, and/or the adenosine triphosphate sulfurylase may be coupled to a solid support as described above. In some embodiments, the polymerase is coupled to a solid support. In some embodiments, the luciferase is coupled to a solid support.

Suitable supports include organic or inorganic materials and may be of any suitable size or shape (e.g., scaffolds sheets, platforms, and/or nanoparticles). Tethering or immobilizing the components of the assays according to the present application serves to, for example, confine them spatially as well as to enhance their stability and/or function in carrying out, for example, a cascading or sequential reaction as part of the particular assay. In certain embodiments, the support materials include, e.g., nucleotide sequences or gels. In certain embodiments, the enzymes or components of the assays according to the present application may be immobilized on or tethered to, for example, a nanoparticle or the luminal surface of a channel (e.g., a microfluidic channel) of a support material such as a platform.

Several techniques can be used to immobilize components of an assay according to the present application (e.g., enzymes) on surfaces. For example, components may be attached non-specifically or be bound through specific, though non-oriented, chemical reactions (such as carboxy-amide binding). Oriented enzyme immobilization may also be used in accordance with methods of the present application. Oriented enzyme immobilization confers several advantages including, for example, positioning a binding tag (e.g., an affinity tag) so that the activity and stability of the tethered enzyme is optimized (see Mukai et al., “Sequential Reactions of Surface-Tethered Glycolytic Enzymes,” Chem. Biol. 16(9):1013-20 (2009), which is hereby incorporated by reference in its entirety).

One example of how an enzyme involved in nucleic acid detection would be tethered to a surface is the use of oriented immobilization. In certain embodiments of the assays according to the present application, recombinant enzymes or assay components which are involved in the assay's reactions are engineered with an affinity tag, enabling them to bind to a surface such as silica or nickel, or a component of a surface such as nickel-nitrilotriacetic acid. For example, an affinity tag could be attached at the amino or carboxy terminus of a protein to be immobilized, or be embedded within the protein to be immobilized. The optimal location of the tethering domain will depend upon the nature and location of the enzyme's catalytic domain(s), substrate binding domain(s), and any conformational changes the enzyme must make.

The use of affinity tagged proteins is especially convenient as the proteins (i.e., DNA polymerase, luciferase, etc.) used in the methods of the present application can readily be expressed as fusions with a suitable binding tag to facilitate immobilization to solid support containing the corresponding capture binding moiety. Suitable capture moieties and binding tag partners that can be used in accordance with this embodiment of the present application include, without limitation, His-Si, His, Si, biotin, streptavidin, Pt, Au, Ag, His-Pt, His-Au, His-Ag, GST, antibody, and epitope tag. Methods of covalently attaching oligonucleotides to a solid support are well known in the art, see e.g., Gosh et al., “Covalent Attachment of Oligonucleotides to Solid Supports,” Nucleic Acids Res. 15(13): 5353-5372 (1987), Joos et al., “Covalent Attachment of Hybridizable Oligonucleotides to Glass Supports,” Anal. Biochem. 247(1):96-101 (1997); Lund et al., “Assessment of Methods for Covalent Binding of Nucleic Acids to Magnetic Beads, Dynabeads, and the Characteristics of the Bound Nucleic Acids in Hybridization Reactions,” Nucleic Acids Res. 16(22):10861-80 (1988), which are hereby incorporated by reference in their entirety.

In certain embodiments, the polymerase and/or the luciferase is coupled to a solid support.

In accordance with any aspect of the present application, the polymerase and/or the luciferase may be coupled to the solid support with a linker selected from the group consisting of His-Si, His, Si, biotin, streptavidin, Pt, Au, Ag, His-Pt, His-Au, His-Ag, GST, antibody, and epitope tag.

The surfaces acting as a support, platform, or scaffold can take multiple forms, including, for example, various nanoparticles, or strands of nucleic acids, and may include various geometries.

In certain embodiments according to the present application, the support is a nanoparticle. As used herein, the term “nanoparticle” refers to any particle the average diameter of which is in the nanometer range, i.e., having an average diameter up to 1 μm. The nanoparticle used can be made of any suitable organic or inorganic matter that will be known to those of ordinary skill in the art. For example, nanoparticles may be composed of any polymer, iron (KIM oxide, gold, silver, carbon, silica, CdSe and/or CdS. In one embodiment, the nanoparticle is a magnetic nanoparticle. In another embodiment, the nanoparticle is a magnetic, silica-coated nanoparticle (MSP).

In addition to nanoparticles (NP), supports or scaffolds of different materials can be in the form of rods, planar surfaces, graphene sheets, nanotubes, DNA scaffolds, gels, microspheres, or inner channel walls of a microchannel of a larger support. Quantum dots are also contemplated for use as a support in accordance with the present application. Enzyme immobilization can be attained via non-specific binding, chemical modifications, affinity tags, or other conjugation techniques.

In one embodiment according to the present application, the methods comprise carrying out a positive and/or negative control. Detection of a diagnostic or prognostic amount of a target nucleic acid is carried out by comparison with a control amount. A control amount of a target nucleic acid can be any amount or a range of amount which is to be compared against a test amount of a target nucleic acid. A control amount may be the amount of a target nucleic acid in a positive or negative control sample carried out as part of the assay according to the present application. A control amount can be either an absolute amount (e.g., μg/ml) or a relative amount (e.g., relative intensity of signals).

Exemplary negative controls for use in the methods of the present application include a reaction where one or more enzymes and/or ID-Oligos are excluded. In some embodiments the negative control is a blocking oligonucleotide which targets the ID-Oligo or “primer” (modified at the 3′/5′ end to inhibit extension), ribonuclease (RNase) or DNase added to the reaction mixture, a reaction mixture lacking the primer(s), a reaction mixture lacking nucleotides (dNTPs), and a reaction mixture lacking any of the substrates or enzymes. Exemplary positive controls for use in the methods of the present application include various concentrations (including saturating amounts) of target viral genome or fragment thereof (single-stranded DNA or single-stranded RNA oligonucleotide having (i) an identical or substantially identical sequence to at least the portion of the viral genome that is complementary (or capable of hybridizing) to primer(s) provided in connection with embodiments described herein and (ii) a further single-stranded sequence to serve as a template enabling extension by the polymerase); and pre-annealed double-stranded DNA or RNA/DNA with overhanging single-stranded sequence enabling extension by the polymerase. Accordingly, in some embodiments, the method further involves comparing the readout signal to that of a control reaction that lacks the target viral genome. In some embodiments, the concentration of the target viral genome in the sample is determined.

Accordingly, in some embodiments of the methods of the disclosure, the presence or absence of a target is determined by comparison to a positive and/or negative control. For example, absence of a target viral genome in a provided sample is indicated where a readout signal is not produced/detected for the provided sample according to the methods described herein and (i) a readout signal is produced/detected for a positive control and/or (ii) a readout signal is not produced/detected for a negative control. Alternatively, for example, presence of a target viral genome in a provided sample is indicated where a readout signal is produced/detected for the provided sample according to the methods described herein and (i) a readout signal is produced/detected for a positive control and/or (ii) a readout signal is not produced/detected for a negative control.

In some embodiments, the sample is a biological sample. In some embodiments the sample comprises or consists of blood, serum, plasma, blood products (e.g., red blood cells, white blood cells, or other blood products), urine, cerebrospinal fluid, saliva, sputum, mucus/cells from nasopharyngeal, oropharyngeal, or nasal-mid-turbinate swabs, tissue, fecal matter or sewage, or a synthetic material.

In some embodiments, the sample contains a free target at least partially single-stranded viral genome. In accordance with such embodiments, the sample can be directly contacted with the oligonucleotide molecule (i.e., the ID-Oligo) so that the oligonucleotide molecule hybridizes to the selected portion of the target at least partially single-stranded viral genome and forms a hybridization product comprising a double-stranded nucleic acid start portion in the viral genome at a location corresponding to the selected portion and the single-stranded template region is adjacent to the double-stranded nucleic acid start portion. Thus, in some embodiments, the provided sample is not subjected to a treatment to isolate or extract nucleic acids from the sample. In such embodiments, genomic viral DNA or RNA present in the sample (e.g., free or circulating genomic viral DNA or RNA) is subjected to the methods of the disclosure.

In some embodiments, the sample contains a target at least partially single-stranded viral genome that is enclosed within or complexed with a viral structure or viral protein (e.g., a viral envelope, a viral capsid, or is complexed with a viral nucleocapsid protein). In accordance with such embodiments, the target at least partially single-stranded viral genome is extracted from the viral structure or viral protein using one or more methods known to those skilled in the art. For example, the target at least partially single-stranded viral genome may be purified from a viral structure or viral protein using Trizol reagent and/or MagMAX Viral/Pathogen II Nucleic Acid Isolation Kit (Applied Biosystems, CA); Maxwell® Viral Total Nucleic Acid Purification Kits; Qiagen QIAamp Viral RNA Mini Kit; Qiagen MinElute Virus Spin Kit; QIAGEN with QIAmp DSP Viral RNA Mini Kit; QIAGEN EZ1 Advanced XL with EZ1 DSP Virus Kit; QIAGEN QIAcube with QIAmp DSP Viral RNA Mini Kit; Roche MagNA Pure LC with Total Nucleic Acid Kit; Roche MagNA Pure Compact with Nucleic Acid Isolation Kit; Roche MagNA Pure 96 with DNA and Viral NA Small Volume Kit; QIAGEN RNeasy Mini KIts. Likewise, the sample may also be subjected to steps to extract or isolate the genomic viral DNA or RNA from other components of the sample (e.g., viral membrane envelope). For example, nucleic acids may be isolated or separated from other sample components according to known techniques for nucleic acid isolation (e.g., phenol-chloroform extraction, guanidinium-acid-phenol extraction, density gradient centrifugation using cesium chloride or cesium trifluoroacetate, glass fiber filtration, and magnetic bead separation). In certain embodiments, the sample is contacted with one or more reagents to, e.g., break down the viral membrane. Such reagents include phospholipid-disrupting enzymes and/or detergents (e.g., phospholipases and/or alpha-hemolysin) to break down the viral membrane, as well as proteases (e.g., proteinase K) to break open the viral capsid.

In some embodiments, providing a sample containing a target at least partially single-stranded viral genome involves collecting the sample from a subject. In some embodiments, the subject is a human, non-human primate, cat, dog, ferret, pig, goat, sheep, cattle, chicken, turkey, duck, camelid, reptile, tiger, bat, pangolin, or amphibian.

In some embodiments, providing a sample containing a target at least partially single-stranded viral genome involves collecting the sample ex vivo from a surface, material, or inanimate object.

Various compounds and components may interfere with the methods described herein. For example, FIG. 12 demonstrates the inhibitory effect of sodium azide on the luminescence output of the coupled ATPS-luciferase reaction shown in FIG. 4. Thus, in some embodiments, the sample is free of compounds and components that inhibit the luminescence output of luciferase. Accordingly, the sample may be free of sodium-azide, sodium-citrate, heparin, EDTA, EGTA, proteases, phosphates, 2,2′-azino-di-(3-ethylbenzothiazoline)-6-sulfonic acid (ABTS), and/or butylated hydroxytoluene (BHT). In various related aspects, the present application also relates to devices and kits for performing the methods described herein. Such kits contain monitors, reagents and procedures that can be utilized in a clinical or research setting or adapted for either the field laboratory or on-site use. In particular, kits comprising the disclosed reagents used in practicing the methods described herein include any of a number of means for detecting the captured target nucleic acid molecule and measuring the bioluminescent signal produced subsequent to target capture, along with appropriate instructions, are contemplated. Suitable kits comprise reagents sufficient for performing an assay, or one or more independent steps of an assay, to detect a target nucleic acid molecule, as described herein above.

It is to be understood that such a kit is useful for any of the methods of the present application. The choice of particular components is dependent upon the particular method the kit is designed to carry out. Additional components can be provided for detection of the analytical output, as measured by the release of ATP and detection of the bioluminescent signal.

As described above, the kit optionally further comprises instructions for detecting the target nucleic acid nucleic acid by the methods described herein. The instructions present in such a kit instruct the user on how to use the components of the kit to perform the various methods of the present application. These instructions can include a description of the detection methods of the present application, including detection by luminescence.

Accordingly, another aspect of the present disclosure is a kit for detecting a target at least partially single-stranded viral genome in a sample. The kit includes: a polymerase; a dNTP mixture; one or more enzymes for producing adenosine triphosphates from released free phosphates; and a luciferase for producing a bioluminescent readout signal. In some embodiments, the kit includes: an oligonucleotide molecule complementary to a selected portion of the target at least partially single-stranded viral genome adjacent to a single-stranded template region of the target at least partially single-stranded viral genome.

Suitable oligonucleotide molecules complementary to a selected portion of the target at least partially single-stranded viral genome (also referred to herein as ID-Oligos or primers), as well as adjacent single-stranded template regions, useful in connection with this aspect of the disclosure are described in connection with the methods of the disclosure.

In some embodiments, the single-stranded template region is a uracil/thymine-free region. Such single-stranded template regions are described herein above.

In some embodiments, the kit comprises multiple oligonucleotide molecules for hybridizing to different complementary portions of the target at least partially single-stranded viral genome adjacent to different single-stranded template regions of the target at least partially single-stranded viral genome. In some embodiments, one or more of the different single-stranded template regions is/are uracil/thymine-free regions. In some embodiments, all of the different single-stranded template regions are uracil/thymine-free regions.

The kits described above may also comprise multiple primers for detecting multiple target nucleic acid molecules and/or multiple fragments thereof. In a contemplated kit for multiplexed primer-mediated specific nucleic acid detection, the kit contains a plurality of primers for nucleic acid targets of interest. Preferably, where the kits contain multiple primers, each of the primers is designed to interrogate a different target nucleic acid sequence or a different fragment thereof.

Suitable polymerases useful in connection with this aspect of the disclosure are described in connection with the methods described herein. In some embodiments, the polymerase is a reverse transcriptase. In some embodiments, the polymerase is a DNA polymerase. The polymerase may be coupled to a support (e.g., a solid support), as described in accordance with the methods of the disclosure.

Suitable enzymes for producing adenosine triphosphates from released free phosphates useful in connection with this aspect of the disclosure are described in connection with the methods described herein. In some embodiments, the one or more enzymes for producing adenosine triphosphates from released free phosphates is/are coupled to one or more supports. In some embodiments, the one or more enzymes for producing adenosine triphosphates from released free phosphates includes glyceraldehyde 3-phosphate dehydrogenase and phosphoglycerate kinase. In some embodiments, glyceraldehyde 3-phosphate dehydrogenase and the phosphoglycerate kinase are coupled to a support (e.g., a solid support). As described above, contacting released free phosphates with adenosine 5′-phosphosulfate in the presence of adenosine triphosphate sulfurylase produces adenosine triphosphate. Accordingly, in some embodiments, the one or more enzymes for producing adenosine triphosphates includes adenosine triphosphate sulfurylase and the kit further includes adenosine 5′-phosphosulfate. In some embodiments, the adenosine triphosphate sulfurylase is coupled to a support (e.g., a solid support).

Suitable luciferase enzymes useful in connection with this aspect of the disclosure are described in connection with the methods described herein. The luciferase may be coupled to a support (e.g., a solid support), as described in accordance with the methods of the disclosure.

In some embodiments, the kit further includes a device. The device may be, for example, a microfluidic device (e.g., a chip, cartridge, or the like) comprising microfluidic channels, partitions, and/or reaction chambers. The device may also be, for example, a solid surface (e.g., plate) comprising one or more wells. The device may also be, for example, a chromatography device (e.g., a paper chromatography or thin-layer chromatography device). In some embodiments, the device includes at least one channel extending from an inlet, the inlet configured to receive a sample as described herein. In some embodiments, the channel includes reagents used in practicing the methods described herein. The sample moves by, e.g., capillary action through the channel, thereby contacting the reagents described herein to (if the target is present in the sample) result in release of free phosphates to produce a readout signal, as described herein. In some embodiments, the device is in the form of a solid support (e.g., plate or the like) comprising one or more wells. In some embodiments, the device includes at least one well having or connected to an inlet or opening, the inlet or opening configured to receive a sample as described herein. In some embodiments, the well includes reagents used in practicing the methods described herein. The well is configured to receive the sample and thereby the sample contacts reagents described herein to (if the target is present in the sample) result in release of free phosphates to produce a readout signal, as described herein. The device may also include an optical window or display for displaying an optical indication of luminescence/presence of the target in the sample. The device may also include an optical window or display for displaying an optical indication of no detected luminescence/absence of the target in the sample.

As discussed herein, in some embodiments, a reader (e.g., a luminometer) is used to detect a readout signal. Accordingly, in some embodiments, the device according to embodiments of the disclosure comprises a reader. In some embodiments, the reader is separate from the device according to embodiments of the disclosure (e.g., a stand-alone reader). In some embodiments, the reader is an integral part of a substrate for use with the device (e.g., a single-use or a reusable substrate such as a card or cartridge). The reader may detect a signal produced by a reaction of the presently claimed invention. In some embodiments, the reader detects luminescence produced by the enzymatic reactions carried out within the device. The detection of the signal by reader may indicate the presence of a target single-stranded viral genome. In some embodiments, the reader compares the signal produced by a control reaction with the signal produced by the sample reaction to generate an indication of luminescence/presence of the target in the sample as compared to the control (e.g., a positive or negative control).

The device may also be configured with more than one channel, well, or the like, each including reagents used in practicing the methods described herein. In this way, each channel may be used to detect presence or absence of a target. The target for detection may be independently selected for each channel and may be directed to, for example, the same or different virus, different variants of a virus, or duplicative targets. In some embodiments, at least one of the channels is utilized for a control as described herein.

Another aspect of the disclosure is a reaction mixture. The reaction mixture includes: a target at least partially single-stranded viral genome; one or more oligonucleotide molecules, each oligonucleotide molecule complementary to a selected portion of the target at least partially single-stranded viral genome adjacent to a single-stranded template region of the target at least partially single-stranded viral genome; a hybridization product comprising a double-stranded nucleic acid start portion in the viral genome at a location corresponding to the selected portion and the single-stranded template region is adjacent to the double-stranded nucleic acid start portion; a polymerase; and a dNTP mixture. In one embodiment, the dNTP mixture is substantially free of an unmodified form of dATP. In one embodiment, the dNTP mixture comprises dATP, where the dATP is an alpha-phosphate modified adenosine triphosphate (e.g., dATPαS). In some embodiments, the single-stranded template region is a uracil/thymine-free region. Such single-stranded template regions are described herein above.

Another aspect of the disclosure is a reaction mixture. The reaction mixture includes: one or more oligonucleotide molecules, each oligonucleotide molecule complementary to a selected portion of a target at least partially single-stranded viral genome adjacent to a single-stranded template region of the target at least partially single-stranded viral genome; a polymerase; and a dNTP mixture. In one embodiment, the dNTP mixture is substantially free of an unmodified form of dATP. In one embodiment, the dNTP mixture comprises dATP, where the dATP is an alpha-phosphate modified adenosine triphosphate (e.g., dATPαS). In some embodiments, the single-stranded template region is a uracil/thymine-free region. Such single-stranded template regions are described herein above.

In some embodiments, the reaction mixtures described herein above include free phosphates. In some embodiments, the reaction mixture includes glyceraldehyde 3-phosphate dehydrogenase and phosphoglycerate kinase. In some embodiments, the reaction mixture includes a luciferase.

Another aspect of the disclosure is a method of treating a human. The method involves: obtaining a sample from a human; determining a presence or an absence of at least a portion of a single-stranded viral genome in the sample by one or more of the methods of detecting a target described herein; and treating the human based on the presence or absence of at least a portion of the single-stranded genome in the sample.

Another aspect of the disclosure is a method of treating a non-human subject. The method involves: obtaining a sample from a non-human subject; determining a presence or an absence of at least a portion of a single-stranded viral genome in the sample by one or more of the methods of detecting a target described herein; and treating the non-human subject based on the presence or absence of at least a portion of the single-stranded genome in the sample.

As used herein the term “therapeutically effective amount” is an amount of an active agent which confers a therapeutic effect on the treated subject, which may be prophylactic or retroactive to the onset of the condition being treated. The therapeutic effect may be objective (e.g., measurable by a quantitative or qualitative test or marker) or subjective (e.g., subject gives an indication of or feels an effect or physician observes a change).

As used herein the terms “treat,” “treated,” and “treating” refer to both therapeutic treatment and prophylactic or preventative measures, having an objective of protecting (partially or wholly) against, reversing, or slowing down (e.g., reduce or postpone the onset of) an undesired physiological condition, disorder or disease, or to obtain beneficial or desired clinical results such as partial or total restoration or inhibition in decline of a parameter, value, function or result that had or would become abnormal. For the purposes of this application, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (partial or total), whether or not it translates to immediate lessening of actual clinical symptoms, or enhancement or improvement of the condition, disorder or disease. Treatment seeks to elicit a clinically significant response without excessive levels of adverse side effects. The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

In certain embodiments, the subject described herein is an animal. In certain embodiments, the subject is a mammal. In certain embodiments, the subject is a human. In certain embodiments, the subject is a non-human animal. In certain embodiments, the subject is a non-human mammal. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In another embodiment, the subject is a research animal such as a rodent, dog, or non-human primate. In certain embodiments, the subject is a non-human transgenic animal such as a transgenic mouse or transgenic pig.

In some embodiments, the treatment involves administering a therapeutically effective amount of a suitable agent (e.g., an antiviral agent, an antiparasitic agent, or an immunomodulatory agent) to the subject to treat (including, e.g., associated signs and/or symptoms of infection) a viral infection in the subject. Examples of suitable antiviral agents include, but are not limited to, oseltamivir (e.g., TAMIFLU™), zanamivir (e.g., RELENZA™) amantadine, rimantadine, remdesivir, chloroquine, ritonavir, lopinavir, ribavirin, penciclovir, nitazoxanide, nafamostat, favipiravir, corticosteroids, and any combination thereof. Examples of suitable antiparasitic agents include avermectins (e.g., ivermectin); benzimidazoles (e.g., fenbendazole, albendazole, mebendazole), praziquantel, metronidazole, tinidazole, Pyrimidines (e.g., pyrantel, morantel); organophosphates (e.g., dichlorvos, trichlorfom, coumaphos); imidazothiazoles (e.g., levamisole, tetramisole, butamisole); chloroquine, doxycycline, piperazine, amprolium, ronidazole (e.g., BELGA®, RIDSOL-S®, RONIDA®, RONIVET®). Suitable immunomodulatory agents include antibody therapies, immune globulins, corticosteroids (dexamethasone); baricitinib (e.g., OLUMIANT®), oclacitinib (e.g., APOQUEL®), azathioprine (e.g., IMURAN®), 6-mercaptopurine, anakinra (e.g., KINERET®), siltuximab (e.g., SYLVANT®), interferon alpha (IFNα), interferon beta (IFNβ), acalabrutinib (e.g., CALQUENCE®), ibrutinib (e.g., IMBRUVICA®), zanubrutinib (e.g., BRUKINSA®), ruxolitinib (e.g., JAKAFI®), tofacitinib (e.g., XELJANZ XR®); and convalescent plasma.

The agent may be administered alone or in combination with other therapies (e.g., second antiviral, antiparasitic, and/or immunomodulatory agents). The treatment may be administered to a subject by any route sufficient to achieve therapeutic systemic circulation levels, for example, orally, intranasally, rectally, topically, transmucosally, intravenously, or other parenteral routes of administration such as subcutaneous, intravenous injection (IV), intramuscular injection (IM), intrathecal injection (IT), or intraperitoneal injection (IP). A therapeutically effective amount of the therapy may be administered in a single dose or in multiple doses at pre-determined intervals over time that when added together provides the therapeutically effective amount.

It will be understood that the specific dose level and frequency of dosage required to provide a therapeutic effect in any particular subject may be varied based upon a variety of factors such as, but not limited to, species, age, body weight, general health, gender, diet, mode of administration, time of administration, rate of metabolism (e.g., half-life), co-administration with one or more other drugs, and severity of the particular condition.

TABLE 9
Sequence Table

| SEQ | Sequence Information (each GenBank Accession No. provided includes additional |
|---|---|
| ID NO: | sequence information and is hereby incorporated by reference in its entirety) |
| 1 | GenBank accession no. NC_045512 (Severe acute respiratory syndrome coronavirus 2 |
| | isolate Wuhan-Hu-1, complete genome) |
| | ATTAAAGGTTTATACCTTCCCAGGTAACAAACCAACCAACTTTCGATCTCTTGTAGATCTGTTCTCT |
| | AAACGAACTTTAAAATCTGTGTGGCTGTCACTCGGCTGCATGCTTAGTGCACTCACGCAGTATAATT |
| | AATAACTAATTACTGTCGTTGACAGGACACGAGTAACTCGTCTATCTTCTGCAGGCTGCTTACGGTT |
| | TCGTCCGTGTTGCAGCCGATCATCAGCACATCTAGGTTTCGTCCGGGTGTGACCGAAAGGTAAGATG |
| | GAGAGCCTTGTCCCTGGTTTCAACGAGAAAACACACGTCCAACTCAGTTTGCCTGTTTTACAGGTTC |
| | GCGACGTGCTCGTACGTGGCTTTGGAGACTCCGTGGAGGAGGTCTTATCAGAGGCACGTCAACATCT |
| | TAAAGATGGCACTTGTGGCTTAGTAGAAGTTGAAAAAGGCGTTTTGCCTCAACTTGAACAGCCCTAT |
| | GTGTTCATCAAACGTTCGGATGCTCGAACTGCACCTCATGGTCATGTTATGGTTGAGCTGGTAGCAG |
| | AACTCGAAGGCATTCAGTACGGTCGTAGTGGTGAGACACTTGGTGTCCTTGTCCCTCATGTGGGCGA |
| | AATACCAGTGGCTTACCGCAAGGTTCTTCTTCGTAAGAACGGTAATAAAGGAGCTGGTGGCCATAGT |
| | TACGGCGCCGATCTAAAGTCATTTGACTTAGGCGACGAGCTTGGCACTGATCCTTATGAAGATTTTC |
| | AAGAAAACTGGAACACTAAACATAGCAGTGGTGTTACCCGTGAACTCATGCGTGAGCTTAACGGAGG |
| | GGCATACACTCGCTATGTCGATAACAACTTCTGTGGCCCTGATGGCTACCCTCTTGAGTGCATTAAA |
| | GACCTTCTAGCACGTGCTGGTAAAGCTTCATGCACTTTGTCCGAACAACTGGACTTTATTGACACTA |
| | AGAGGGGTGTATACTGCTGCCGTGAACATGAGCATGAAATTGCTTGGTACACGGAACGTTCTGAAAA |
| | GAGCTATGAATTGCAGACACCTTTTGAAATTAAATTGGCAAAGAAATTTGACACCTTCAATGGGGAA |
| | TGTCCAAATTTTGTATTTCCCTTAAATTCCATAATCAAGACTATTCAACCAAGGGTTGAAAAGAAAA |
| | AGCTTGATGGCTTTATGGGTAGAATTCGATCTGTCTATCCAGTTGCGTCACCAAATGAATGCAACCA |
| | AATGTGCCTTTCAACTCTCATGAAGTGTGATCATTGTGGTGAAACTTCATGGCAGACGGGCGATTTT |
| | GTTAAAGCCACTTGCGAATTTTGTGGCACTGAGAATTTGACTAAAGAAGGTGCCACTACTTGTGGTT |
| | ACTTACCCCAAAATGCTGTTGTTAAAATTTATTGTCCAGCATGTCACAATTCAGAAGTAGGACCTGA |
| | GCATAGTCTTGCCGAATACCATAATGAATCTGGCTTGAAAACCATTCTTCGTAAGGGTGGTCGCACT |
| | ATTGCCTTTGGAGGCTGTGTGTTCTCTTATGTTGGTTGCCATAACAAGTGTGCCTATTGGGTTCCAC |
| | GTGCTAGCGCTAACATAGGTTGTAACCATACAGGTGTTGTTGGAGAAGGTTCCGAAGGTCTTAATGA |
| | CAACCTTCTTGAAATACTCCAAAAAGAGAAAGTCAACATCAATATTGTTGGTGACTTTAAACTTAAT |
| | GAAGAGATCGCCATTATTTTGGCATCTTTTTCTGCTTCCACAAGTGCTTTTGTGGAAACTGTGAAAG |
| | GTTTGGATTATAAAGCATTCAAACAAATTGTTGAATCCTGTGGTAATTTTAAAGTTACAAAAGGAAA |
| | AGCTAAAAAAGGTGCCTGGAATATTGGTGAACAGAAATCAATACTGAGTCCTCTTTATGCATTTGCA |
| | TCAGAGGCTGCTCGTGTTGTACGATCAATTTTCTCCCGCACTCTTGAAACTGCTCAAAATTCTGTGC |
| | GTGTTTTACAGAAGGCCGCTATAACAATACTAGATGGAATTTCACAGTATTCACTGAGACTCATTGA |
| | TGCTATGATGTTCACATCTGATTTGGCTACTAACAATCTAGTTGTAATGGCCTACATTACAGGTGGT |
| | GTTGTTCAGTTGACTTCGCAGTGGCTAACTAACATCTTTGGCACTGTTTATGAAAAACTCAAACCCG |
| | TCCTTGATTGGCTTGAAGAGAAGTTTAAGGAAGGTGTAGAGTTTCTTAGAGACGGTTGGGAAATTGT |
| | TAAATTTATCTCAACCTGTGCTTGTGAAATTGTCGGTGGACAAATTGTCACCTGTGCAAAGGAAATT |
| | AAGGAGAGTGTTCAGACATTCTTTAAGCTTGTAAATAAATTTTTGGCTTTGTGTGCTGACTCTATCA |
| | TTATTGGTGGAGCTAAACTTAAAGCCTTGAATTTAGGTGAAACATTTGTCACGCACTCAAAGGGATT |
| | GTACAGAAAGTGTGTTAAATCCAGAGAAGAAACTGGCCTACTCATGCCTCTAAAAGCCCCAAAAGAA |
| | ATTATCTTCTTAGAGGGAGAAACACTTCCCACAGAAGTGTTAACAGAGGAAGTTGTCTTGAAAACTG |
| | GTGATTTACAACCATTAGAACAACCTACTAGTGAAGCTGTTGAAGCTCCATTGGTTGGTACACCAGT |
| | TTGTATTAACGGGCTTATGTTGCTCGAAATCAAAGACACAGAAAAGTACTGTGCCCTTGCACCTAAT |
| | ATGATGGTAACAAACAATACCTTCACACTCAAAGGCGGTGCACCAACAAAGGTTACTTTTGGTGATG |
| | ACACTGTGATAGAAGTGCAAGGTTACAAGAGTGTGAATATCACTTTTGAACTTGATGAAAGGATTGA |
| | TAAAGTACTTAATGAGAAGTGCTCTGCCTATACAGTTGAACTCGGTACAGAAGTAAATGAGTTCGCC |
| | TGTGTTGTGGCAGATGCTGTCATAAAAACTTTGCAACCAGTATCTGAATTACTTACACCACTGGGCA |
| | TTGATTTAGATGAGTGGAGTATGGCTACATACTACTTATTTGATGAGTCTGGTGAGTTTAAATTGGC |
| | TTCACATATGTATTGTTCTTTCTACCCTCCAGATGAGGATGAAGAAGAAGGTGATTGTGAAGAAGAA |
| | GAGTTTGAGCCATCAACTCAATATGAGTATGGTACTGAAGATGATTACCAAGGTAAACCTTTGGAAT |
| | TTGGTGCCACTTCTGCTGCTCTTCAACCTGAAGAAGAGCAAGAAGAAGATTGGTTAGATGATGATAG |
| | TCAACAAACTGTTGGTCAACAAGACGGCAGTGAGGACAATCAGACAACTACTATTCAAACAATTGTT |
| | GAGGTTCAACCTCAATTAGAGATGGAACTTACACCAGTTGTTCAGACTATTGAAGTGAATAGTTTTA |
| | GTGGTTATTTAAAACTTACTGACAATGTATACATTAAAAATGCAGACATTGTGGAAGAAGCTAAAAA |
| | GGTAAAACCAACAGTGGTTGTTAATGCAGCCAATGTTTACCTTAAACATGGAGGAGGTGTTGCAGGA |
| | GCCTTAAATAAGGCTACTAACAATGCCATGCAAGTTGAATCTGATGATTACATAGCTACTAATGGAC |
| | CACTTAAAGTGGGTGGTAGTTGTGTTTTAAGCGGACACAATCTTGCTAAACACTGTCTTCATGTTGT |
| | CGGCCCAAATGTTAACAAAGGTGAAGACATTCAACTTCTTAAGAGTGCTTATGAAAATTTTAATCAG |
| | CACGAAGTTCTACTTGCACCATTATTATCAGCTGGTATTTTTGGTGCTGACCCTATACATTCTTTAA |
| | GAGTTTGTGTAGATACTGTTCGCACAAATGTCTACTTAGCTGTCTTTGATAAAAATCTCTATGACAA |
| | ACTTGTTTCAAGCTTTTTGGAAATGAAGAGTGAAAAGCAAGTTGAACAAAAGATCGCTGAGATTCCT |
| | AAAGAGGAAGTTAAGCCATTTATAACTGAAAGTAAACCTTCAGTTGAACAGAGAAAACAAGATGATA |
| | AGAAAATCAAAGCTTGTGTTGAAGAAGTTACAACAACTCTGGAAGAAACTAAGTTCCTCACAGAAAA |
| | CTTGTTACTTTATATTGACATTAATGGCAATCTTCATCCAGATTCTGCCACTCTTGTTAGTGACATT |
| | GACATCACTTTCTTAAAGAAAGATGCTCCATATATAGTGGGTGATGTTGTTCAAGAGGGTGTTTTAA |
| | CTGCTGTGGTTATACCTACTAAAAAGGCTGGTGGCACTACTGAAATGCTAGCGAAAGCTTTGAGAAA |
| | AGTGCCAACAGACAATTATATAACCACTTACCCGGGTCAGGGTTTAAATGGTTACACTGTAGAGGAG |
| | GCAAAGACAGTGCTTAAAAAGTGTAAAAGTGCCTTTTACATTCTACCATCTATTATCTCTAATGAGA |
| | AGCAAGAAATTCTTGGAACTGTTTCTTGGAATTTGCGAGAAATGCTTGCACATGCAGAAGAAACACG |
| | CAAATTAATGCCTGTCTGTGTGGAAACTAAAGCCATAGTTTCAACTATACAGCGTAAATATAAGGGT |
| | ATTAAAATACAAGAGGGTGTGGTTGATTATGGTGCTAGATTTTACTTTTACACCAGTAAAACAACTG |
| | TAGCGTCACTTATCAACACACTTAACGATCTAAATGAAACTCTTGTTACAATGCCACTTGGCTATGT |
| | AACACATGGCTTAAATTTGGAAGAAGCTGCTCGGTATATGAGATCTCTCAAAGTGCCAGCTACAGTT |
| | TCTGTTTCTTCACCTGATGCTGTTACAGCGTATAATGGTTATCTTACTTCTTCTTCTAAAACACCTG |
| | AAGAACATTTTATTGAAACCATCTCACTTGCTGGTTCCTATAAAGATTGGTCCTATTCTGGACAATC |
| | TACACAACTAGGTATAGAATTTCTTAAGAGAGGTGATAAAAGTGTATATTACACTAGTAATCCTACC |
| | ACATTCCACCTAGATGGTGAAGTTATCACCTTTGACAATCTTAAGACACTTCTTTCTTTGAGAGAAG |
| | TGAGGACTATTAAGGTGTTTACAACAGTAGACAACATTAACCTCCACACGCAAGTTGTGGACATGTC |
| | AATGACATATGGACAACAGTTTGGTCCAACTTATTTGGATGGAGCTGATGTTACTAAAATAAAACCT |
| | CATAATTCACATGAAGGTAAAACATTTTATGTTTTACCTAATGATGACACTCTACGTGTTGAGGCTT |
| | TTGAGTACTACCACACAACTGATCCTAGTTTTCTGGGTAGGTACATGTCAGCATTAAATCACACTAA |
| | AAAGTGGAAATACCCACAAGTTAATGGTTTAACTTCTATTAAATGGGCAGATAACAACTGTTATCTT |
| | GCCACTGCATTGTTAACACTCCAACAAATAGAGTTGAAGTTTAATCCACCTGCTCTACAAGATGCTT |
| | ATTACAGAGCAAGGGCTGGTGAAGCTGCTAACTTTTGTGCACTTATCTTAGCCTACTGTAATAAGAC |
| | AGTAGGTGAGTTAGGTGATGTTAGAGAAACAATGAGTTACTTGTTTCAACATGCCAATTTAGATTCT |
| | TGCAAAAGAGTCTTGAACGTGGTGTGTAAAACTTGTGGACAACAGCAGACAACCCTTAAGGGTGTAG |
| | AAGCTGTTATGTACATGGGCACACTTTCTTATGAACAATTTAAGAAAGGTGTTCAGATACCTTGTAC |
| | GTGTGGTAAACAAGCTACAAAATATCTAGTACAACAGGAGTCACCTTTTGTTATGATGTCAGCACCA |
| | CCTGCTCAGTATGAACTTAAGCATGGTACATTTACTTGTGCTAGTGAGTACACTGGTAATTACCAGT |
| | GTGGTCACTATAAACATATAACTTCTAAAGAAACTTTGTATTGCATAGACGGTGCTTTACTTACAAA |
| | GTCCTCAGAATACAAAGGTCCTATTACGGATGTTTTCTACAAAGAAAACAGTTACACAACAACCATA |
| | AAACCAGTTACTTATAAATTGGATGGTGTTGTTTGTACAGAAATTGACCCTAAGTTGGACAATTATT |
| | ATAAGAAAGACAATTCTTATTTCACAGAGCAACCAATTGATCTTGTACCAAACCAACCATATCCAAA |
| | CGCAAGCTTCGATAATTTTAAGTTTGTATGTGATAATATCAAATTTGCTGATGATTTAAACCAGTTA |
| | ACTGGTTATAAGAAACCTGCTTCAAGAGAGCTTAAAGTTACATTTTTCCCTGACTTAAATGGTGATG |
| | TGGTGGCTATTGATTATAAACACTACACACCCTCTTTTAAGAAAGGAGCTAAATTGTTACATAAACC |
| | TATTGTTTGGCATGTTAACAATGCAACTAATAAAGCCACGTATAAACCAAATACCTGGTGTATACGT |
| | TGTCTTTGGAGCACAAAACCAGTTGAAACATCAAATTCGTTTGATGTACTGAAGTCAGAGGACGCGC |
| | AGGGAATGGATAATCTTGCCTGCGAAGATCTAAAACCAGTCTCTGAAGAAGTAGTGGAAAATCCTAC |
| | CATACAGAAAGACGTTCTTGAGTGTAATGTGAAAACTACCGAAGTTGTAGGAGACATTATACTTAAA |
| | CCAGCAAATAATAGTTTAAAAATTACAGAAGAGGTTGGCCACACAGATCTAATGGCTGCTTATGTAG |
| | ACAATTCTAGTCTTACTATTAAGAAACCTAATGAATTATCTAGAGTATTAGGTTTGAAAACCCTTGC |
| | TACTCATGGTTTAGCTGCTGTTAATAGTGTCCCTTGGGATACTATAGCTAATTATGCTAAGCCTTTT |
| | CTTAACAAAGTTGTTAGTACAACTACTAACATAGTTACACGGTGTTTAAACCGTGTTTGTACTAATT |
| | ATATGCCTTATTTCTTTACTTTATTGCTACAATTGTGTACTTTTACTAGAAGTACAAATTCTAGAAT |
| | TAAAGCATCTATGCCGACTACTATAGCAAAGAATACTGTTAAGAGTGTCGGTAAATTTTGTCTAGAG |
| | GCTTCATTTAATTATTTGAAGTCACCTAATTTTTCTAAACTGATAAATATTATAATTTGGTTTTTAC |
| | TATTAAGTGTTTGCCTAGGTTCTTTAATCTACTCAACCGCTGCTTTAGGTGTTTTAATGTCTAATTT |
| | AGGCATGCCTTCTTACTGTACTGGTTACAGAGAAGGCTATTTGAACTCTACTAATGTCACTATTGCA |
| | ACCTACTGTACTGGTTCTATACCTTGTAGTGTTTGTCTTAGTGGTTTAGATTCTTTAGACACCTATC |
| | CTTCTTTAGAAACTATACAAATTACCATTTCATCTTTTAAATGGGATTTAACTGCTTTTGGCTTAGT |
| | TGCAGAGTGGTTTTTGGCATATATTCTTTTCACTAGGTTTTTCTATGTACTTGGATTGGCTGCAATC |
| | ATGCAATTGTTTTTCAGCTATTTTGCAGTACATTTTATTAGTAATTCTTGGCTTATGTGGTTAATAA |
| | TTAATCTTGTACAAATGGCCCCGATTTCAGCTATGGTTAGAATGTACATCTTCTTTGCATCATTTTA |
| | TTATGTATGGAAAAGTTATGTGCATGTTGTAGACGGTTGTAATTCATCAACTTGTATGATGTGTTAG |
| | AAACGTAATAGAGCAACAAGAGTCGAATGTACAACTATTGTTAATGGTGTTAGAAGGTCCTTTTATG |
| | TCTATGCTAATGGAGGTAAAGGCTTTTGCAAACTACACAATTGGAATTGTGTTAATTGTGATACATT |
| | CTGTGCTGGTAGTACATTTATTAGTGATGAAGTTGCGAGAGACTTGTCACTACAGTTTAAAAGACCA |
| | ATAAATCCTACTGACCAGTCTTCTTACATCGTTGATAGTGTTACAGTGAAGAATGGTTCCATCCATC |
| | TTTACTTTGATAAAGCTGGTCAAAAGACTTATGAAAGACATTCTCTCTCTCATTTTGTTAACTTAGA |
| | CAACCTGAGAGCTAATAACACTAAAGGTTCATTGCCTATTAATGTTATAGTTTTTGATGGTAAATCA |
| | AAATGTGAAGAATCATCTGCAAAATCAGCGTCTGTTTACTACAGTCAGCTTATGTGTCAACCTATAC |
| | TGTTACTAGATCAGGCATTAGTGTCTGATGTTGGTGATAGTGCGGAAGTTGCAGTTAAAATGTTTGA |
| | TGCTTACGTTAATACGTTTTCATCAACTTTTAACGTACCAATGGAAAAACTCAAAACACTAGTTGCA |
| | ACTGCAGAAGCTGAACTTGCAAAGAATGTGTCCTTAGACAATGTCTTATCTACTTTTATTTCAGCAG |
| | CTCGGCAAGGGTTTGTTGATTCAGATGTAGAAACTAAAGATGTTGTTGAATGTCTTAAATTGTCACA |
| | TCAATCTGACATAGAAGTTACTGGCGATAGTTGTAATAACTATATGCTCACCTATAACAAAGTTGAA |
| | AACATGACACCCCGTGACCTTGGTGCTTGTATTGACTGTAGTGCGCGTCATATTAATGCGCAGGTAG |
| | CAAAAAGTCACAACATTGCTTTGATATGGAACGTTAAAGATTTCATGTCATTGTCTGAACAACTACG |
| | AAAACAAATACGTAGTGCTGCTAAAAAGAATAACTTACCTTTTAAGTTGACATGTGCAACTACTAGA |
| | CAAGTTGTTAATGTTGTAACAACAAAGATAGCACTTAAGGGTGGTAAAATTGTTAATAATTGGTTGA |
| | AGCAGTTAATTAAAGTTACACTTGTGTTCCTTTTTGTTGCTGCTATTTTCTATTTAATAACACCTGT |
| | TCATGTCATGTCTAAACATACTGACTTTTCAAGTGAAATCATAGGATACAAGGCTATTGATGGTGGT |
| | GTCACTCGTGACATAGCATCTACAGATACTTGTTTTGCTAACAAACATGCTGATTTTGACACATGGT |
| | TTAGCCAGCGTGGTGGTAGTTATACTAATGACAAAGCTTGCCCATTGATTGCTGCAGTCATAACAAG |
| | AGAAGTGGGTTTTGTCGTGCCTGGTTTGCCTGGCACGATATTACGCACAACTAATGGTGACTTTTTG |
| | CATTTCTTACCTAGAGTTTTTAGTGCAGTTGGTAACATCTGTTACACACCATCAAAACTTATAGAGT |
| | ACACTGACTTTGCAACATCAGCTTGTGTTTTGGCTGCTGAATGTACAATTTTTAAAGATGCTTCTGG |
| | TAAGCCAGTACCATATTGTTATGATACCAATGTACTAGAAGGTTCTGTTGCTTATGAAAGTTTACGC |
| | CCTGACACACGTTATGTGCTCATGGATGGCTCTATTATTCAATTTCCTAACACCTACCTTGAAGGTT |
| | CTGTTAGAGTGGTAACAACTTTTGATTCTGAGTACTGTAGGCACGGCACTTGTGAAAGATCAGAAGC |
| | TGGTGTTTGTGTATCTACTAGTGGTAGATGGGTACTTAACAATGATTATTACAGATCTTTACCAGGA |
| | GTTTTCTGTGGTGTAGATGCTGTAAATTTACTTACTAATATGTTTACACCACTAATTCAACCTATTG |
| | GTGCTTTGGACATATCAGCATCTATAGTAGCTGGTGGTATTGTAGCTATCGTAGTAACATGCCTTGC |
| | CTACTATTTTATGAGGTTTAGAAGAGCTTTTGGTGAATACAGTCATGTAGTTGCCTTTAATACTTTA |
| | CTATTCCTTATGTCATTCACTGTACTCTGTTTAACACCAGTTTACTCATTCTTACCTGGTGTTTATT |
| | CTGTTATTTACTTGTACTTGACATTTTATCTTACTAATGATGTTTCTTTTTTAGCACATATTCAGTG |
| | GATGGTTATGTTCACACCTTTAGTACCTTTCTGGATAACAATTGCTTATATCATTTGTATTTCCACA |
| | AAGCATTTCTATTGGTTCTTTAGTAATTACCTAAAGAGACGTGTAGTCTTTAATGGTGTTTCCTTTA |
| | GTACTTTTGAAGAAGCTGCGCTGTGCACCTTTTTGTTAAATAAAGAAATGTATCTAAAGTTGCGTAG |
| | TGATGTGCTATTACCTCTTACGCAATATAATAGATACTTAGCTCTTTATAATAAGTACAAGTATTTT |
| | AGTGGAGCAATGGATACAACTAGCTACAGAGAAGCTGCTTGTTGTCATCTCGCAAAGGCTCTCAATG |
| | ACTTCAGTAACTCAGGTTCTGATGTTCTTTACCAACCACCACAAACCTCTATCACCTCAGCTGTTTT |
| | GCAGAGTGGTTTTAGAAAAATGGCATTCCCATCTGGTAAAGTTGAGGGTTGTATGGTACAAGTAACT |
| | TGTGGTACAACTACACTTAACGGTCTTTGGCTTGATGACGTAGTTTACTGTCCAAGACATGTGATCT |
| | GCACCTCTGAAGACATGCTTAACCCTAATTATGAAGATTTACTCATTCGTAAGTCTAATCATAATTT |
| | CTTGGTACAGGCTGGTAATGTTCAACTCAGGGTTATTGGACATTCTATGCAAAATTGTGTACTTAAG |
| | CTTAAGGTTGATACAGCCAATCCTAAGACACCTAAGTATAAGTTTGTTCGCATTCAACCAGGACAGA |
| | CTTTTTCAGTGTTAGCTTGTTACAATGGTTCACCATCTGGTGTTTACCAATGTGCTATGAGGCCCAA |
| | TTTCACTATTAAGGGTTCATTCCTTAATGGTTCATGTGGTAGTGTTGGTTTTAACATAGATTATGAC |
| | TGTGTCTCTTTTTGTTACATGCACCATATGGAATTACCAACTGGAGTTCATGCTGGCACAGACTTAG |
| | AAGGTAACTTTTATGGACCTTTTGTTGACAGGCAAACAGCACAAGCAGCTGGTACGGACACAACTAT |
| | TACAGTTAATGTTTTAGCTTGGTTGTACGCTGCTGTTATAAATGGAGACAGGTGGTTTCTCAATCGA |
| | TTTACCACAACTCTTAATGACTTTAACCTTGTGGCTATGAAGTACAATTATGAACCTCTAACACAAG |
| | ACCATGTTGACATACTAGGACCTCTTTCTGCTCAAACTGGAATTGCCGTTTTAGATATGTGTGCTTC |
| | ATTAAAAGAATTACTGCAAAATGGTATGAATGGACGTACCATATTGGGTAGTGCTTTATTAGAAGAT |
| | GAATTTACACCTTTTGATGTTGTTAGACAATGCTCAGGTGTTACTTTCCAAAGTGCAGTGAAAAGAA |
| | CAATCAAGGGTACACACCACTGGTTGTTACTCACAATTTTGACTTCACTTTTAGTTTTAGTCCAGAG |
| | TACTCAATGGTCTTTGTTCTTTTTTTTGTATGAAAATGCCTTTTTACCTTTTGCTATGGGTATTATT |
| | GCTATGTCTGCTTTTGCAATGATGTTTGTCAAACATAAGCATGCATTTCTCTGTTTGTTTTTGTTAC |
| | CTTCTCTTGCCACTGTAGCTTATTTTAATATGGTCTATATGCCTGCTAGTTGGGTGATGCGTATTAT |
| | GACATGGTTGGATATGGTTGATACTAGTTTGTCTGGTTTTAAGCTAAAAGACTGTGTTATGTATGCA |
| | TCAGCTGTAGTGTTACTAATCCTTATGACAGCAAGAACTGTGTATGATGATGGTGCTAGGAGAGTGT |
| | GGACACTTATGAATGTCTTGACACTCGTTTATAAAGTTTATTATGGTAATGCTTTAGATCAAGCCAT |
| | TTCCATGTGGGCTCTTATAATCTCTGTTACTTCTAACTACTCAGGTGTAGTTACAACTGTCATGTTT |
| | TTGGCCAGAGGTATTGTTTTTATGTGTGTTGAGTATTGCCCTATTTTCTTCATAACTGGTAATACAC |
| | TTCAGTGTATAATGCTAGTTTATTGTTTCTTAGGCTATTTTTGTACTTGTTACTTTGGCCTCTTTTG |
| | TTTACTCAACCGCTACTTTAGACTGACTCTTGGTGTTTATGATTACTTAGTTTCTACACAGGAGTTT |
| | AGATATATGAATTCACAGGGACTACTCCCACCCAAGAATAGCATAGATGCCTTCAAACTCAACATTA |
| | AATTGTTGGGTGTTGGTGGCAAACCTTGTATCAAAGTAGCCACTGTACAGTCTAAAATGTCAGATGT |
| | AAAGTGCACATCAGTAGTCTTACTCTCAGTTTTGCAACAACTCAGAGTAGAATCATCATCTAAATTG |
| | TGGGCTCAATGTGTCCAGTTACACAATGACATTCTCTTAGCTAAAGATACTACTGAAGCCTTTGAAA |
| | AAATGGTTTCACTACTTTCTGTTTTGCTTTCCATGCAGGGTGCTGTAGACATAAACAAGCTTTGTGA |
| | AGAAATGCTGGACAACAGGGCAACCTTACAAGCTATAGCCTCAGAGTTTAGTTCCCTTCCATCATAT |
| | GCAGCTTTTGCTACTGCTCAAGAAGCTTATGAGCAGGCTGTTGCTAATGGTGATTCTGAAGTTGTTC |
| | TTAAAAAGTTGAAGAAGTCTTTGAATGTGGCTAAATCTGAATTTGACCGTGATGCAGCCATGCAACG |
| | TAAGTTGGAAAAGATGGCTGATCAAGCTATGACCCAAATGTATAAACAGGCTAGATCTGAGGACAAG |
| | AGGGCAAAAGTTACTAGTGCTATGCAGACAATGCTTTTCACTATGCTTAGAAAGTTGGATAATGATG |
| | CACTCAACAACATTATCAACAATGCAAGAGATGGTTGTGTTCCCTTGAACATAATACCTCTTACAAC |
| | AGCAGCCAAACTAATGGTTGTCATACCAGACTATAACACATATAAAAATACGTGTGATGGTACAACA |
| | TTTACTTATGCATCAGCATTGTGGGAAATCCAACAGGTTGTAGATGCAGATAGTAAAATTGTTCAAC |
| | TTAGTGAAATTAGTATGGACAATTCACCTAATTTAGCATGGCCTCTTATTGTAACAGCTTTAAGGGC |
| | CAATTCTGCTGTCAAATTACAGAATAATGAGCTTAGTCCTGTTGCACTACGACAGATGTCTTGTGCT |
| | GCCGGTACTACACAAACTGCTTGCACTGATGACAATGCGTTAGCTTACTACAACACAACAAAGGGAG |
| | GTAGGTTTGTACTTGCACTGTTATCCGATTTACAGGATTTGAAATGGGCTAGATTCCCTAAGAGTGA |
| | TGGAACTGGTACTATCTATACAGAACTGGAACCACCTTGTAGGTTTGTTACAGACACACCTAAAGGT |
| | CCTAAAGTGAAGTATTTATACTTTATTAAAGGATTAAACAACCTAAATAGAGGTATGGTACTTGGTA |
| | GTTTAGCTGCCACAGTACGTCTACAAGCTGGTAATGCAACAGAAGTGCCTGCCAATTCAACTGTATT |
| | ATCTTTCTGTGCTTTTGCTGTAGATGCTGCTAAAGCTTACAAAGATTATCTAGCTAGTGGGGGACAA |
| | CCAATCACTAATTGTGTTAAGATGTTGTGTACACACACTGGTACTGGTCAGGCAATAACAGTTACAC |
| | CGGAAGCCAATATGGATCAAGAATCCTTTGGTGGTGCATCGTGTTGTCTGTACTGCCGTTGCCACAT |
| | AGATCATCCAAATCCTAAAGGATTTTGTGACTTAAAAGGTAAGTATGTACAAATACCTACAACTTGT |
| | GCTAATGACCCTGTGGGTTTTACACTTAAAAACACAGTCTGTACCGTCTGCGGTATGTGGAAAGGTT |
| | ATGGCTGTAGTTGTGATCAACTCCGCGAACCCATGCTTCAGTCAGCTGATGCACAATCGTTTTTAAA |
| | CGGGTTTGCGGTGTAAGTGCAGCCCGTCTTACACCGTGCGGCACAGGCACTAGTACTGATGTCGTAT |
| | ACAGGGCTTTTGACATCTACAATGATAAAGTAGCTGGTTTTGCTAAATTCCTAAAAACTAATTGTTG |
| | TCGCTTCCAAGAAAAGGACGAAGATGACAATTTAATTGATTCTTACTTTGTAGTTAAGAGACACACT |
| | TTCTCTAACTACCAACATGAAGAAACAATTTATAATTTACTTAAGGATTGTCCAGCTGTTGCTAAAC |
| | ATGACTTCTTTAAGTTTAGAATAGACGGTGACATGGTACCACATATATCACGTCAACGTCTTACTAA |
| | ATACACAATGGCAGACCTCGTCTATGCTTTAAGGCATTTTGATGAAGGTAATTGTGACACATTAAAA |
| | GAAATACTTGTCACATACAATTGTTGTGATGATGATTATTTCAATAAAAAGGACTGGTATGATTTTG |
| | TAGAAAACCCAGATATATTACGCGTATACGCCAACTTAGGTGAACGTGTACGCCAAGCTTTGTTAAA |
| | AACAGTACAATTCTGTGATGCCATGCGAAATGCTGGTATTGTTGGTGTACTGACATTAGATAATCAA |
| | GATCTCAATGGTAACTGGTATGATTTCGGTGATTTCATACAAACCACGCCAGGTAGTGGAGTTCCTG |
| | TTGTAGATTCTTATTATTCATTGTTAATGCCTATATTAACCTTGACCAGGGCTTTAACTGCAGAGTC |
| | ACATGTTGACACTGACTTAACAAAGCCTTACATTAAGTGGGATTTGTTAAAATATGACTTCACGGAA |
| | GAGAGGTTAAAACTCTTTGACCGTTATTTTAAATATTGGGATCAGACATACCACCCAAATTGTGTTA |
| | ACTGTTTGGATGACAGATGCATTCTGCATTGTGCAAACTTTAATGTTTTATTCTCTACAGTGTTCCC |
| | ACCTACAAGTTTTGGACCACTAGTGAGAAAAATATTTGTTGATGGTGTTCCATTTGTAGTTTCAACT |
| | GGATACCACTTCAGAGAGCTAGGTGTTGTACATAATCAGGATGTAAACTTACATAGCTCTAGACTTA |
| | GTTTTAAGGAATTACTTGTGTATGCTGCTGACCCTGCTATGCACGCTGCTTCTGGTAATCTATTACT |
| | AGATAAACGCACTACGTGCTTTTCAGTAGCTGCACTTACTAACAATGTTGCTTTTCAAACTGTCAAA |
| | CCCGGTAATTTTAACAAAGACTTCTATGACTTTGCTGTGTCTAAGGGTTTCTTTAAGGAAGGAAGTT |
| | CTGTTGAATTAAAACACTTCTTCTTTGCTCAGGATGGTAATGCTGCTATCAGCGATTATGACTACTA |
| | TCGTTATAATCTACCAACAATGTGTGATATCAGACAACTACTATTTGTAGTTGAAGTTGTTGATAAG |
| | TACTTTGATTGTTACGATGGTGGCTGTATTAATGCTAACCAAGTCATCGTCAACAACCTAGACAAAT |
| | CAGCTGGTTTTCCATTTAATAAATGGGGTAAGGCTAGACTTTATTATGATTCAATGAGTTATGAGGA |
| | TCAAGATGCACTTTTCGCATATACAAAACGTAATGTCATCCCTACTATAACTCAAATGAATCTTAAG |
| | TATGCCATTAGTGCAAAGAATAGAGCTCGCACCGTAGCTGGTGTCTCTATCTGTAGTACTATGACCA |
| | ATAGACAGTTTCATCAAAAATTATTGAAATCAATAGCCGCCACTAGAGGAGCTACTGTAGTAATTGG |
| | AACAAGCAAATTCTATGGTGGTTGGCACAACATGTTAAAAACTGTTTATAGTGATGTAGAAAACCCT |
| | CACCTTATGGGTTGGGATTATCCTAAATGTGATAGAGCCATGCCTAACATGCTTAGAATTATGGCCT |
| | CACTTGTTCTTGCTCGCAAACATACAACGTGTTGTAGCTTGTCACACCGTTTCTATAGATTAGCTAA |
| | TGAGTGTGCTCAAGTATTGAGTGAAATGGTCATGTGTGGCGGTTCACTATATGTTAAACCAGGTGGA |
| | ACCTCATCAGGAGATGCCACAACTGCTTATGCTAATAGTGTTTTTAACATTTGTCAAGCTGTCACGG |
| | CCAATGTTAATGCACTTTTATCTACTGATGGTAACAAAATTGCCGATAAGTATGTCCGCAATTTACA |
| | ACACAGACTTTATGAGTGTCTCTATAGAAATAGAGATGTTGACACAGACTTTGTGAATGAGTTTTAG |
| | GCATATTTGCGTAAACATTTCTCAATGATGATACTCTCTGACGATGCTGTTGTGTGTTTCAATAGCA |
| | CTTATGCATCTCAAGGTCTAGTGGCTAGCATAAAGAACTTTAAGTCAGTTCTTTATTATCAAAACAA |
| | TGTTTTTATGTCTGAAGCAAAATGTTGGACTGAGACTGACCTTACTAAAGGACCTCATGAATTTTGC |
| | TCTCAACATACAATGCTAGTTAAACAGGGTGATGATTATGTGTACCTTCCTTACCCAGATCCATCAA |
| | GAATCCTAGGGGCCGGCTGTTTTGTAGATGATATCGTAAAAACAGATGGTACACTTATGATTGAACG |
| | GTTCGTGTCTTTAGCTATAGATGCTTACCCACTTACTAAACATCCTAATCAGGAGTATGCTGATGTC |
| | TTTCATTTGTACTTACAATACATAAGAAAGCTAGATGATGAGTTAACAGGACACATGTTAGACATGT |
| | ATTCTGTTATGCTTACTAATGATAACACTTCAAGGTATTGGGAACCTGAGTTTTATGAGGCTATGTA |
| | CACACCGCATACAGTCTTACAGGCTGTTGGGGCTTGTGTTCTTTGCAATTCACAGACTTCATTAAGA |
| | TGTGGTGCTTGCATACGTAGACCATTCTTATGTTGTAAATGCTGTTACGACCATGTCATATCAACAT |
| | CACATAAATTAGTCTTGTCTGTTAATCCGTATGTTTGCAATGCTCCAGGTTGTGATGTCACAGATGT |
| | GACTCAACTTTACTTAGGAGGTATGAGCTATTATTGTAAATCACATAAACCACCCATTAGTTTTCCA |
| | TTGTGTGCTAATGGACAAGTTTTTGGTTTATATAAAAATACATGTGTTGGTAGCGATAATGTTACTG |
| | ACTTTAATGCAATTGCAACATGTGACTGGACAAATGCTGGTGATTACATTTTAGCTAACACCTGTAC |
| | TGAAAGACTCAAGCTTTTTGCAGCAGAAACGCTCAAAGCTACTGAGGAGACATTTAAACTGTCTTAT |
| | GGTATTGCTACTGTACGTGAAGTGCTGTCTGACAGAGAATTACATCTTTCATGGGAAGTTGGTAAAC |
| | CTAGACCACCACTTAACCGAAATTATGTCTTTACTGGTTATCGTGTAACTAAAAACAGTAAAGTACA |
| | AATAGGAGAGTACACCTTTGAAAAAGGTGACTATGGTGATGCTGTTGTTTACCGAGGTACAACAACT |
| | TACAAATTAAATGTTGGTGATTATTTTGTGCTGACATCACATACAGTAATGCCATTAAGTGCACCTA |
| | CACTAGTGCCACAAGAGCACTATGTTAGAATTACTGGCTTATACCCAACACTCAATATCTCAGATGA |
| | GTTTTCTAGCAATGTTGCAAATTATCAAAAGGTTGGTATGCAAAAGTATTCTACACTCCAGGGACCA |
| | CCTGGTACTGGTAAGAGTCATTTTGCTATTGGCCTAGCTCTCTACTACCCTTCTGCTCGCATAGTGT |
| | ATACAGCTTGCTCTCATGCCGCTGTTGATGCACTATGTGAGAAGGCATTAAAATATTTGCCTATAGA |
| | TAAATGTAGTAGAATTATACCTGCACGTGCTCGTGTAGAGTGTTTTGATAAATTCAAAGTGAATTCA |
| | ACATTAGAACAGTATGTCTTTTGTACTGTAAATGCATTGCCTGAGACGACAGCAGATATAGTTGTCT |
| | TTGATGAAATTTCAATGGCCACAAATTATGATTTGAGTGTTGTCAATGCCAGATTACGTGCTAAGCA |
| | CTATGTGTACATTGGCGACCCTGCTCAATTACCTGCACCACGCACATTGCTAACTAAGGGCACACTA |
| | GAACCAGAATATTTCAATTCAGTGTGTAGACTTATGAAAACTATAGGTCCAGACATGTTCCTCGGAA |
| | CTTGTCGGCGTTGTCCTGCTGAAATTGTTGACACTGTGAGTGCTTTGGTTTATGATAATAAGCTTAA |
| | AGCACATAAAGACAAATCAGCTCAATGCTTTAAAATGTTTTATAAGGGTGTTATCACGCATGATGTT |
| | TCATCTGCAATTAACAGGCCACAAATAGGCGTGGTAAGAGAATTCCTTACACGTAACCCTGCTTGGA |
| | GAAAAGCTGTCTTTATTTCACCTTATAATTCACAGAATGCTGTAGCCTCAAAGATTTTGGGACTACC |
| | AACTCAAACTGTTGATTCATCACAGGGCTCAGAATATGACTATGTCATATTCACTCAAACCACTGAA |
| | ACAGCTCACTCTTGTAATGTAAACAGATTTAATGTTGCTATTACCAGAGCAAAAGTAGGCATACTTT |
| | GCATAATGTCTGATAGAGACCTTTATGACAAGTTGCAATTTACAAGTCTTGAAATTCCACGTAGGAA |
| | TGTGGCAACTTTACAAGCTGAAAATGTAACAGGACTCTTTAAAGATTGTAGTAAGGTAATCACTGGG |
| | TTACATCCTACACAGGCACCTACACACCTCAGTGTTGACACTAAATTCAAAACTGAAGGTTTATGTG |
| | TTGACATACCTGGCATACCTAAGGACATGACCTATAGAAGACTCATCTCTATGATGGGTTTTAAAAT |
| | GAATTATCAAGTTAATGGTTACCCTAACATGTTTATCACCCGCGAAGAAGCTATAAGACATGTACGT |
| | GCATGGATTGGCTTCGATGTCGAGGGGTGTCATGCTACTAGAGAAGCTGTTGGTACCAATTTACCTT |
| | TACAGCTAGGTTTTTCTACAGGTGTTAACCTAGTTGCTGTACCTACAGGTTATGTTGATACACCTAA |
| | TAATACAGATTTTTCCAGAGTTAGTGCTAAACCACCGCCTGGAGATCAATTTAAACACCTCATACCA |
| | CTTATGTACAAAGGACTTCCTTGGAATGTAGTGCGTATAAAGATTGTACAAATGTTAAGTGACACAC |
| | TTAAAAATCTCTCTGACAGAGTCGTATTTGTCTTATGGGCACATGGCTTTGAGTTGACATCTATGAA |
| | GTATTTTGTGAAAATAGGACCTGAGCGCACCTGTTGTCTATGTGATAGACGTGCCACATGCTTTTCC |
| | ACTGCTTCAGACACTTATGCCTGTTGGCATCATTCTATTGGATTTGATTACGTCTATAATCCGTTTA |
| | TGATTGATGTTCAACAATGGGGTTTTACAGGTAACCTACAAAGCAACCATGATCTGTATTGTCAAGT |
| | CCATGGTAATGCACATGTAGCTAGTTGTGATGCAATCATGACTAGGTGTCTAGCTGTCCACGAGTGC |
| | TTTGTTAAGCGTGTTGACTGGACTATTGAATATCCTATAATTGGTGATGAACTGAAGATTAATGCGG |
| | CTTGTAGAAAGGTTCAACACATGGTTGTTAAAGCTGCATTATTAGCAGACAAATTCCCAGTTCTTCA |
| | CGACATTGGTAACCCTAAAGCTATTAAGTGTGTACCTCAAGCTGATGTAGAATGGAAGTTCTATGAT |
| | GCACAGCCTTGTAGTGACAAAGCTTATAAAATAGAAGAATTATTCTATTCTTATGCCACACATTCTG |
| | ACAAATTCACAGATGGTGTATGCCTATTTTGGAATTGCAATGTCGATAGATATCCTGCTAATTCCAT |
| | TGTTTGTAGATTTGACACTAGAGTGCTATCTAACCTTAACTTGCCTGGTTGTGATGGTGGCAGTTTG |
| | TATGTAAATAAACATGCATTCCACACACCAGCTTTTGATAAAAGTGCTTTTGTTAATTTAAAACAAT |
| | TACCATTTTTCTATTACTCTGACAGTCCATGTGAGTCTCATGGAAAACAAGTAGTGTCAGATATAGA |
| | TTATGTACCACTAAAGTCTGCTACGTGTATAACACGTTGCAATTTAGGTGGTGCTGTCTGTAGACAT |
| | CATGCTAATGAGTACAGATTGTATCTCGATGCTTATAACATGATGATCTCAGCTGGCTTTAGCTTGT |
| | GGGTTTACAAACAATTTGATACTTATAACCTCTGGAACACTTTTACAAGACTTCAGAGTTTAGAAAA |
| | TGTGGCTTTTAATGTTGTAAATAAGGGACACTTTGATGGACAACAGGGTGAAGTACCAGTTTCTATC |
| | ATTAATAACACTGTTTACACAAAAGTTGATGGTGTTGATGTAGAATTGTTTGAAAATAAAACAACAT |
| | TACCTGTTAATGTAGCATTTGAGCTTTGGGCTAAGCGCAACATTAAACCAGTACCAGAGGTGAAAAT |
| | ACTCAATAATTTGGGTGTGGACATTGCTGCTAATACTGTGATCTGGGACTACAAAAGAGATGCTCCA |
| | GCACATATATCTACTATTGGTGTTTGTTCTATGACTGACATAGCCAAGAAACCAACTGAAACGATTT |
| | GTGCACCACTCACTGTCTTTTTTGATGGTAGAGTTGATGGTCAAGTAGACTTATTTAGAAATGCCCG |
| | TAATGGTGTTCTTATTACAGAAGGTAGTGTTAAAGGTTTACAACCATCTGTAGGTCCCAAACAAGCT |
| | AGTCTTAATGGAGTCACATTAATTGGAGAAGCCGTAAAAACACAGTTCAATTATTATAAGAAAGTTG |
| | ATGGTGTTGTCCAACAATTACCTGAAACTTACTTTACTCAGAGTAGAAATTTACAAGAATTTAAACC |
| | CAGGAGTCAAATGGAAATTGATTTCTTAGAATTAGCTATGGATGAATTCATTGAACGGTATAAATTA |
| | GAAGGCTATGCCTTCGAACATATCGTTTATGGAGATTTTAGTCATAGTCAGTTAGGTGGTTTACATC |
| | TACTGATTGGACTAGCTAAACGTTTTAAGGAATCACCTTTTGAATTAGAAGATTTTATTCCTATGGA |
| | CAGTACAGTTAAAAACTATTTCATAACAGATGCGCAAACAGGTTCATCTAAGTGTGTGTGTTCTGTT |
| | ATTGATTTATTACTTGATGATTTTGTTGAAATAATAAAATCCCAAGATTTATCTGTAGTTTCTAAGG |
| | TTGTCAAAGTGACTATTGACTATACAGAAATTTCATTTATGCTTTGGTGTAAAGATGGCCATGTAGA |
| | AACATTTTACCCAAAATTACAATCTAGTCAAGCGTGGCAACCGGGTGTTGCTATGCCTAATCTTTAC |
| | AAAATGCAAAGAATGCTATTAGAAAAGTGTGACCTTCAAAATTATGGTGATAGTGCAACATTACCTA |
| | AAGGCATAATGATGAATGTCGCAAAATATACTCAACTGTGTCAATATTTAAACACATTAACATTAGC |
| | TGTACCCTATAATATGAGAGTTATACATTTTGGTGCTGGTTCTGATAAAGGAGTTGCACCAGGTACA |
| | GCTGTTTTAAGACAGTGGTTGCCTACGGGTACGCTGCTTGTCGATTCAGATCTTAATGACTTTGTCT |
| | CTGATGCAGATTCAACTTTGATTGGTGATTGTGCAACTGTACATACAGCTAATAAATGGGATCTCAT |
| | TATTAGTGATATGTACGACCCTAAGACTAAAAATGTTACAAAAGAAAATGACTCTAAAGAGGGTTTT |
| | TTCACTTACATTTGTGGGTTTATACAACAAAAGCTAGCTCTTGGAGGTTCCGTGGCTATAAAGATAA |
| | CAGAACATTCTTGGAATGCTGATCTTTATAAGCTCATGGGACACTTCGCATGGTGGACAGCCTTTGT |
| | TACTAATGTGAATGCGTCATCATCTGAAGCATTTTTAATTGGATGTAATTATCTTGGCAAACCACGC |
| | GAACAAATAGATGGTTATGTCATGCATGCAAATTACATATTTTGGAGGAATACAAATCCAATTCAGT |
| | TGTCTTCCTATTCTTTATTTGACATGAGTAAATTTCCCCTTAAATTAAGGGGTACTGCTGTTATGTC |
| | TTTAAAAGAAGGTCAAATCAATGATATGATTTTATCTCTTCTTAGTAAAGGTAGACTTATAATTAGA |
| | GAAAACAACAGAGTTGTTATTTCTAGTGATGTTCTTGTTAACAACTAAACGAACAATGTTTGTTTTT |
| | CTTGTTTTATTGCCACTAGTCTCTAGTCAGTGTGTTAATCTTACAACCAGAACTCAATTACCCCCTG |
| | CATACACTAATTCTTTCACACGTGGTGTTTATTACCCTGACAAAGTTTTCAGATCCTCAGTTTTACA |
| | TTCAACTCAGGACTTGTTCTTACCTTTCTTTTCCAATGTTACTTGGTTCCATGCTATACATGTCTCT |
| | GGGACCAATGGTACTAAGAGGTTTGATAACCCTGTCCTACCATTTAATGATGGTGTTTATTTTGCTT |
| | CCACTGAGAAGTCTAACATAATAAGAGGCTGGATTTTTGGTACTACTTTAGATTCGAAGACCCAGTC |
| | CCTACTTATTGTTAATAACGCTACTAATGTTGTTATTAAAGTCTGTGAATTTCAATTTTGTAATGAT |
| | CCATTTTTGGGTGTTTATTACCACAAAAACAACAAAAGTTGGATGGAAAGTGAGTTCAGAGTTTATT |
| | CTAGTGCGAATAATTGCACTTTTGAATATGTCTCTCAGCCTTTTCTTATGGACCTTGAAGGAAAACA |
| | GGGTAATTTCAAAAATCTTAGGGAATTTGTGTTTAAGAATATTGATGGTTATTTTAAAATATATTCT |
| | AAGCACACGCCTATTAATTTAGTGCGTGATCTCCCTCAGGGTTTTTCGGCTTTAGAACCATTGGTAG |
| | ATTTGCCAATAGGTATTAACATCACTAGGTTTCAAACTTTACTTGCTTTACATAGAAGTTATTTGAC |
| | TCCTGGTGATTCTTCTTCAGGTTGGACAGCTGGTGCTGCAGCTTATTATGTGGGTTATCTTCAACCT |
| | AGGACTTTTCTATTAAAATATAATGAAAATGGAACCATTACAGATGCTGTAGACTGTGCACTTGACC |
| | CTCTCTCAGAAACAAAGTGTACGTTGAAATCCTTCACTGTAGAAAAAGGAATCTATCAAACTTCTAA |
| | CTTTAGAGTCCAACCAACAGAATCTATTGTTAGATTTCCTAATATTACAAACTTGTGCCCTTTTGGT |
| | GAAGTTTTTAACGCCACCAGATTTGCATCTGTTTATGCTTGGAACAGGAAGAGAATCAGCAACTGTG |
| | TTGCTGATTATTCTGTCCTATATAATTCCGCATCATTTTCCACTTTTAAGTGTTATGGAGTGTCTCC |
| | TACTAAATTAAATGATCTCTGCTTTACTAATGTCTATGCAGATTCATTTGTAATTAGAGGTGATGAA |
| | GTCAGACAAATCGCTCCAGGGCAAACTGGAAAGATTGCTGATTATAATTATAAATTACCAGATGATT |
| | TTACAGGCTGCGTTATAGCTTGGAATTCTAACAATCTTGATTCTAAGGTTGGTGGTAATTATAATTA |
| | CCTGTATAGATTGTTTAGGAAGTCTAATCTCAAACCTTTTGAGAGAGATATTTCAACTGAAATCTAT |
| | CAGGCCGGTAGCACACCTTGTAATGGTGTTGAAGGTTTTAATTGTTACTTTCCTTTACAATCATATG |
| | GTTTCCAACCCACTAATGGTGTTGGTTACCAACCATACAGAGTAGTAGTACTTTCTTTTGAACTTCT |
| | ACATGCACCAGCAACTGTTTGTGGACCTAAAAAGTCTACTAATTTGGTTAAAAACAAATGTGTCAAT |
| | TTCAACTTCAATGGTTTAACAGGCACAGGTGTTCTTACTGAGTCTAACAAAAAGTTTCTGCCTTTCC |
| | AACAATTTGGCAGAGACATTGCTGACACTACTGATGCTGTCCGTGATCCACAGACACTTGAGATTCT |
| | TGACATTACACCATGTTCTTTTGGTGGTGTCAGTGTTATAACACCAGGAACAAATACTTCTAACCAG |
| | GTTGCTGTTCTTTATCAGGATGTTAACTGCACAGAAGTCCCTGTTGCTATTCATGCAGATCAACTTA |
| | CTCCTACTTGGCGTGTTTATTCTACAGGTTCTAATGTTTTTCAAACACGTGCAGGCTGTTTAATAGG |
| | GGCTGAACATGTCAACAACTCATATGAGTGTGACATACCCATTGGTGCAGGTATATGCGCTAGTTAT |
| | CAGACTCAGACTAATTCTCCTCGGCGGGCACGTAGTGTAGCTAGTCAATCCATCATTGCCTACACTA |
| | TGTCACTTGGTGCAGAAAATTCAGTTGCTTACTCTAATAACTCTATTGCCATACCCACAAATTTTAC |
| | TATTAGTGTTACCACAGAAATTCTACCAGTGTCTATGACCAAGACATCAGTAGATTGTACAATGTAC |
| | ATTTGTGGTGATTCAACTGAATGCAGCAATCTTTTGTTGCAATATGGCAGTTTTTGTACACAATTAA |
| | ACCGTGCTTTAACTGGAATAGCTGTTGAACAAGACAAAAACACCCAAGAAGTTTTTGCACAAGTCAA |
| | ACAAATTTACAAAACACCACCAATTAAAGATTTTGGTGGTTTTAATTTTTCACAAATATTACCAGAT |
| | CCATCAAAACCAAGCAAGAGGTCATTTATTGAAGATCTACTTTTCAACAAAGTGACACTTGCAGATG |
| | CTGGCTTCATCAAACAATATGGTGATTGCCTTGGTGATATTGCTGCTAGAGACCTCATTTGTGCACA |
| | AAAGTTTAACGGCCTTACTGTTTTGCCACCTTTGCTCACAGATGAAATGATTGCTCAATACACTTCT |
| | GCACTGTTAGCGGGTACAATCACTTCTGGTTGGACCTTTGGTGCAGGTGCTGCATTACAAATACCAT |
| | TTGCTATGCAAATGGCTTATAGGTTTAATGGTATTGGAGTTACACAGAATGTTCTCTATGAGAACCA |
| | AAAATTGATTGCCAACCAATTTAATAGTGCTATTGGCAAAATTCAAGACTCACTTTCTTCCACAGCA |
| | AGTGCACTTGGAAAACTTCAAGATGTGGTCAACCAAAATGCACAAGCTTTAAACACGCTTGTTAAAC |
| | AACTTAGCTCCAATTTTGGTGCAATTTCAAGTGTTTTAAATGATATCCTTTCACGTCTTGACAAAGT |
| | TGAGGCTGAAGTGCAAATTGATAGGTTGATCACAGGCAGACTTCAAAGTTTGCAGACATATGTGACT |
| | CAACAATTAATTAGAGCTGCAGAAATCAGAGCTTCTGCTAATCTTGCTGCTACTAAAATGTCAGAGT |
| | GTGTACTTGGACAATCAAAAAGAGTTGATTTTTGTGGAAAGGGCTATCATCTTATGTCCTTCCCTCA |
| | GTCAGCACCTCATGGTGTAGTCTTCTTGCATGTGACTTATGTCCCTGCACAAGAAAAGAACTTCACA |
| | ACTGCTCCTGCCATTTGTCATGATGGAAAAGCACACTTTCCTCGTGAAGGTGTCTTTGTTTCAAATG |
| | GCACACACTGGTTTGTAACACAAAGGAATTTTTATGAACCACAAATCATTACTACAGACAACACATT |
| | TGTGTCTGGTAACTGTGATGTTGTAATAGGAATTGTCAACAACACAGTTTATGATCCTTTGCAACCT |
| | GAATTAGACTCATTCAAGGAGGAGTTAGATAAATATTTTAAGAATCATACATCACCAGATGTTGATT |
| | TAGGTGACATCTCTGGCATTAATGCTTCAGTTGTAAACATTCAAAAAGAAATTGACCGCCTCAATGA |
| | GGTTGCCAAGAATTTAAATGAATCTCTCATCGATCTCCAAGAACTTGGAAAGTATGAGCAGTATATA |
| | AAATGGCCATGGTACATTTGGCTAGGTTTTATAGCTGGCTTGATTGCCATAGTAATGGTGACAATTA |
| | TGCTTTGCTGTATGACCAGTTGCTGTAGTTGTCTCAAGGGCTGTTGTTCTTGTGGATCCTGCTGCAA |
| | ATTTGATGAAGACGACTCTGAGCCAGTGCTCAAAGGAGTCAAATTACATTACACATAAACGAACTTA |
| | TGGATTTGTTTATGAGAATCTTCACAATTGGAACTGTAACTTTGAAGCAAGGTGAAATCAAGGATGC |
| | TACTCCTTCAGATTTTGTTCGCGCTACTGCAACGATACCGATACAAGCCTCACTCCCTTTCGGATGG |
| | CTTATTGTTGGCGTTGCACTTCTTGCTGTTTTTCAGAGCGCTTCCAAAATCATAACCCTCAAAAAGA |
| | GATGGCAACTAGCACTCTCCAAGGGTGTTCACTTTGTTTGCAACTTGCTGTTGTTGTTTGTAACAGT |
| | TTACTCACACCTTTTGCTCGTTGCTGCTGGCCTTGAAGCCCCTTTTCTCTATCTTTATGCTTTAGTC |
| | TACTTCTTGCAGAGTATAAACTTTGTAAGAATAATAATGAGGCTTTGGCTTTGCTGGAAATGCCGTT |
| | CCAAAAACCCATTACTTTATGATGCCAACTATTTTCTTTGCTGGCATACTAATTGTTACGACTATTG |
| | TATACCTTACAATAGTGTAACTTCTTCAATTGTCATTACTTCAGGTGATGGCACAACAAGTCCTATT |
| | TCTGAACATGACTACCAGATTGGTGGTTATACTGAAAAATGGGAATCTGGAGTAAAAGACTGTGTTG |
| | TATTACACAGTTACTTCACTTCAGACTATTACCAGCTGTACTCAACTCAATTGAGTACAGACACTGG |
| | TGTTGAACATGTTACCTTCTTCATCTACAATAAAATTGTTGATGAGCCTGAAGAACATGTCCAAATT |
| | CACACAATCGACGGTTCATCCGGAGTTGTTAATCCAGTAATGGAACCAATTTATGATGAACCGACGA |
| | CGACTACTAGCGTGCCTTTGTAAGCACAAGCTGATGAGTACGAACTTATGTACTCATTCGTTTCGGA |
| | AGAGACAGGTACGTTAATAGTTAATAGCGTACTTCTTTTTCTTGCTTTCGTGGTATTCTTGCTAGTT |
| | ACACTAGCCATCCTTACTGCGCTTCGATTGTGTGCGTACTGCTGCAATATTGTTAACGTGAGTCTTG |
| | TAAAACCTTCTTTTTACGTTTACTCTCGTGTTAAAAATCTGAATTCTTCTAGAGTTCCTGATCTTCT |
| | GGTCTAAACGAACTAAATATTATATTAGTTTTTCTGTTTGGAACTTTAATTTTAGCCATGGCAGATT |
| | CCAACGGTACTATTACCGTTGAAGAGCTTAAAAAGCTCCTTGAACAATGGAACCTAGTAATAGGTTT |
| | CCTATTCCTTACATGGATTTGTCTTCTACAATTTGCCTATGCCAACAGGAATAGGTTTTTGTATATA |
| | ATTAAGTTAATTTTCCTCTGGCTGTTATGGCCAGTAACTTTAGCTTGTTTTGTGCTTGCTGCTGTTT |
| | ACAGAATAAATTGGATCACCGGTGGAATTGCTATCGCAATGGCTTGTCTTGTAGGCTTGATGTGGCT |
| | CAGCTACTTCATTGCTTCTTTCAGACTGTTTGCGCGTACGCGTTCCATGTGGTCATTCAATCCAGAA |
| | ACTAACATTCTTCTCAACGTGCCACTCCATGGCACTATTCTGACCAGACCGCTTCTAGAAAGTGAAC |
| | TCGTAATCGGAGCTGTGATCCTTCGTGGACATCTTCGTATTGCTGGACACCATCTAGGACGCTGTGA |
| | CATCAAGGACCTGCCTAAAGAAATCACTGTTGCTACATCACGAACGCTTTCTTATTACAAATTGGGA |
| | GCTTCGCAGCGTGTAGCAGGTGACTCAGGTTTTGCTGCATACAGTCGCTACAGGATTGGCAACTATA |
| | AATTAAACACAGACCATTCCAGTAGCAGTGACAATATTGCTTTGCTTGTACAGTAAGTGACAACAGA |
| | TGTTTCATCTCGTTGACTTTCAGGTTACTATAGCAGAGATATTACTAATTATTATGAGGACTTTTAA |
| | AGTTTCCATTTGGAATCTTGATTACATCATAAACCTCATAATTAAAAATTTATCTAAGTCACTAACT |
| | GAGAATAAATATTCTCAATTAGATGAAGAGCAACCAATGGAGATTGATTAAACGAACATGAAAATTA |
| | TTCTTTTCTTGGCACTGATAACACTCGCTACTTGTGAGCTTTATCACTACCAAGAGTGTGTTAGAGG |
| | TACAACAGTACTTTTAAAAGAACCTTGCTCTTCTGGAACATACGAGGGCAATTCACCATTTCATCCT |
| | CTAGCTGATAACAAATTTGCACTGACTTGCTTTAGCACTCAATTTGCTTTTGCTTGTCCTGACGGCG |
| | TAAAACACGTCTATCAGTTACGTGCCAGATCAGTTTCACCTAAACTGTTCATCAGACAAGAGGAAGT |
| | TCAAGAACTTTACTCTCCAATTTTTCTTATTGTTGCGGCAATAGTGTTTATAACACTTTGCTTCACA |
| | CTCAAAAGAAAGACAGAATGATTGAACTTTCATTAATTGACTTCTATTTGTGCTTTTTAGCCTTTCT |
| | GCTATTCCTTGTTTTAATTATGCTTATTATCTTTTGGTTCTCACTTGAACTGCAAGATCATAATGAA |
| | ACTTGTCACGCCTAAACGAACATGAAATTTCTTGTTTTCTTAGGAATCATCACAACTGTAGCTGCAT |
| | TTCACCAAGAATGTAGTTTACAGTCATGTACTCAACATCAACCATATGTAGTTGATGACCCGTGTCC |
| | TATTCACTTCTATTCTAAATGGTATATTAGAGTAGGAGCTAGAAAATCAGCACCTTTAATTGAATTG |
| | TGCGTGGATGAGGCTGGTTCTAAATCACCCATTCAGTACATCGATATCGGTAATTATACAGTTTCCT |
| | GTTTACCTTTTACAATTAATTGCCAGGAACCTAAATTGGGTAGTCTTGTAGTGCGTTGTTCGTTCTA |
| | TGAAGACTTTTTAGAGTATCATGACGTTCGTGTTGTTTTAGATTTCATCTAAACGAACAAACTAAAA |
| | TGTCTGATAATGGACCCCAAAATCAGCGAAATGCACCCCGCATTACGTTTGGTGGACCCTCAGATTC |
| | AACTGGCAGTAACCAGAATGGAGAACGCAGTGGGGCGCGATCAAAACAACGTCGGCCCCAAGGTTTA |
| | CCCAATAATACTGCGTCTTGGTTCACCGCTCTCACTCAACATGGCAAGGAAGACCTTAAATTCCCTC |
| | GAGGACAAGGCGTTCCAATTAACACCAATAGCAGTCCAGATGACCAAATTGGCTACTACCGAAGAGC |
| | TACCAGACGAATTCGTGGTGGTGACGGTAAAATGAAAGATCTCAGTCCAAGATGGTATTTCTACTAC |
| | CTAGGAACTGGGCCAGAAGCTGGACTTCCCTATGGTGCTAACAAAGACGGCATCATATGGGTTGCAA |
| | CTGAGGGAGCCTTGAATACACCAAAAGATCACATTGGCACCCGCAATCCTGCTAACAATGCTGCAAT |
| | CGTGCTACAACTTCCTCAAGGAACAACATTGCCAAAAGGCTTCTACGCAGAAGGGAGCAGAGGCGGC |
| | AGTCAAGCCTCTTCTCGTTCCTCATCACGTAGTCGCAACAGTTCAAGAAATTCAACTCCAGGCAGCA |
| | GTAGGGGAACTTCTCCTGCTAGAATGGCTGGCAATGGCGGTGATGCTGCTCTTGCTTTGCTGCTGCT |
| | TGACAGATTGAACCAGCTTGAGAGCAAAATGTCTGGTAAAGGCCAACAACAACAAGGCCAAACTGTC |
| | ACTAAGAAATCTGCTGCTGAGGCTTCTAAGAAGCCTCGGCAAAAACGTACTGCCACTAAAGCATACA |
| | ATGTAACACAAGCTTTCGGCAGACGTGGTCCAGAACAAACCCAAGGAAATTTTGGGGACCAGGAACT |
| | AATCAGACAAGGAACTGATTACAAACATTGGCCGCAAATTGCACAATTTGCCCCCAGCGCTTCAGCG |
| | TTCTTCGGAATGTCGCGCATTGGCATGGAAGTCACACCTTCGGGAACGTGGTTGACCTACACAGGTG |
| | CCATCAAATTGGATGACAAAGATCCAAATTTCAAAGATCAAGTCATTTTGCTGAATAAGCATATTGA |
| | CGCATACAAAACATTCCCACCAACAGAGCCTAAAAAGGACAAAAAGAAGAAGGCTGATGAAACTCAA |
| | GCCTTACCGCAGAGACAGAAGAAACAGCAAACTGTGACTCTTCTTCCTGCTGCAGATTTGGATGATT |
| | TCTCCAAACAATTGCAACAATCCATGAGCAGTGCTGACTCAACTCAGGCCTAAACTCATGCAGACCA |
| | CACAAGGCAGATGGGCTATATAAACGTTTTCGCTTTTCCGTTTACGATATATAGTCTACTCTTGTGC |
| | AGAATGAATTCTCGTAACTACATAGCACAAGTAGATGTAGTTAACTTTAATCTCACATAGCAATCTT |
| | TAATCAGTGTGTAACATTAGGGAGGACTTGAAAGAGCCACCACATTTTCACCGAGGCCACGCGGAGT |
| | ACGATCGAGTGTACAGTGAACAATGCTAGGGAGAGCTGCCTATATGGAAGAGCCCTAATGTGTAAAA |
| | TTAATTTTAGTAGTGCTATCCCCATGTGATTTTAATAGCTTCTTAGGAGAATGACAAAAAAAAAAAA |
| | AAAAAAAAAAAAAAAAAAAAA |
| 250 | Reverse Complement of GenBank accession no. NC_045512 (Severe acute respiratory |
| | syndrome coronavirus 2 isolate Wuhan-Hu-1, complete genome) |
| | TTTTTTTTTTTTTTTTTTTTTTTTTTTTTTTTTGTCATTCTCCTAAGAAGCTATTAAAATCACATGG |
| | GGATAGCACTACTAAAATTAATTTTACACATTAGGGCTCTTCCATATAGGCAGCTCTCCCTAGCATT |
| | GTTCACTGTACACTCGATCGTACTCCGCGTGGCCTCGGTGAAAATGTGGTGGCTCTTTCAAGTCCTC |
| | CCTAATGTTACACACTGATTAAAGATTGCTATGTGAGATTAAAGTTAACTACATCTACTTGTGCTAT |
| | GTAGTTAGGAGAATTCATTCTGCACAAGAGTAGACTATATATCGTAAACGGAAAAGCGAAAACGTTT |
| | ATATAGCCCATCTGCCTTGTGTGGTCTGCATGAGTTTAGGCCTGAGTTGAGTCAGCACTGCTCATGG |
| | ATTGTTGCAATTGTTTGGAGAAATCATCCAAATCTGCAGCAGGAAGAAGAGTCACAGTTTGCTGTTT |
| | CTTCTGTCTCTGCGGTAAGGCTTGAGTTTCATCAGCCTTCTTCTTTTTGTCCTTTTTAGGCTCTGTT |
| | GGTGGGAATGTTTTGTATGCGTCAATATGCTTATTCAGCAAAATGACTTGATCTTTGAAATTTGGAT |
| | CTTTGTCATCCAATTTGATGGCACCTGTGTAGGTCAACCACGTTCCCGAAGGTGTGACTTCCATGCC |
| | AATGCGCGACATTCCGAAGAACGCTGAAGCGCTGGGGGCAAATTGTGCAATTTGCGGCCAATGTTTG |
| | TAATCAGTTCCTTGTCTGATTAGTTCCTGGTCCCCAAAATTTCCTTGGGTTTGTTCTGGACCACGTC |
| | TGCCGAAAGCTTGTGTTACATTGTATGCTTTAGTGGCAGTACGTTTTTGCCGAGGCTTCTTAGAAGC |
| | CTCAGCAGCAGATTTCTTAGTGACAGTTTGGCCTTGTTGTTGTTGGCCTTTACCAGACATTTTGCTC |
| | TCAAGCTGGTTCAATCTGTCAAGCAGCAGCAAAGCAAGAGCAGCATCACCGCCATTGCCAGCCATTC |
| | TAGCAGGAGAAGTTCCCCTACTGCTGCCTGGAGTTGAATTTCTTGAACTGTTGCGACTACGTGATGA |
| | GGAACGAGAAGAGGCTTGACTGCCGCCTCTGCTCCCTTCTGCGTAGAAGCCTTTTGGCAATGTTGTT |
| | CCTTGAGGAAGTTGTAGCACGATTGCAGCATTGTTAGCAGGATTGCGGGTGCCAATGTGATCTTTTG |
| | GTGTATTCAAGGCTCCCTCAGTTGCAACCCATATGATGCCGTCTTTGTTAGCACCATAGGGAAGTCC |
| | AGCTTCTGGCCCAGTTCCTAGGTAGTAGAAATACCATCTTGGACTGAGATCTTTCATTTTACCGTCA |
| | CCACCACGAATTCGTCTGGTAGCTCTTCGGTAGTAGCCAATTTGGTCATCTGGACTGCTATTGGTGT |
| | TAATTGGAACGCCTTGTCCTCGAGGGAATTTAAGGTCTTCCTTGCCATGTTGAGTGAGAGCGGTGAA |
| | CCAAGACGCAGTATTATTGGGTAAACCTTGGGGCCGACGTTGTTTTGATCGCGCCCCACTGCGTTCT |
| | CCATTCTGGTTACTGCCAGTTGAATCTGAGGGTCCACCAAACGTAATGCGGGGTGCATTTCGCTGAT |
| | TTTGGGGTCCATTATCAGACATTTTAGTTTGTTCGTTTAGATGAAATCTAAAACAACACGAACGTCA |
| | TGATACTCTAAAAAGTCTTCATAGAACGAACAACGCACTACAAGACTACCCAATTTAGGTTCCTGGC |
| | AATTAATTGTAAAAGGTAAACAGGAAACTGTATAATTACCGATATCGATGTACTGAATGGGTGATTT |
| | AGAACCAGCCTCATCCACGCACAATTCAATTAAAGGTGCTGATTTTCTAGCTCCTACTCTAATATAC |
| | CATTTAGAATAGAAGTGAATAGGACACGGGTCATCAACTACATATGGTTGATGTTGAGTACATGACT |
| | GTAAACTACATTCTTGGTGAAATGCAGCTACAGTTGTGATGATTCCTAAGAAAACAAGAAATTTCAT |
| | GTTCGTTTAGGCGTGACAAGTTTCATTATGATCTTGCAGTTCAAGTGAGAACCAAAAGATAATAAGC |
| | ATAATTAAAACAAGGAATAGCAGAAAGGCTAAAAAGCACAAATAGAAGTCAATTAATGAAAGTTCAA |
| | TCATTCTGTCTTTCTTTTGAGTGTGAAGCAAAGTGTTATAAACACTATTGCCGCAACAATAAGAAAA |
| | ATTGGAGAGTAAAGTTCTTGAACTTCCTCTTGTCTGATGAACAGTTTAGGTGAAACTGATCTGGCAC |
| | GTAACTGATAGACGTGTTTTACGCCGTCAGGACAAGCAAAAGCAAATTGAGTGCTAAAGCAAGTCAG |
| | TGCAAATTTGTTATCAGCTAGAGGATGAAATGGTGAATTGCCCTCGTATGTTCCAGAAGAGCAAGGT |
| | TCTTTTAAAAGTACTGTTGTACCTCTAACACACTCTTGGTAGTGATAAAGCTCACAAGTAGCGAGTG |
| | TTATCAGTGCCAAGAAAAGAATAATTTTCATGTTCGTTTAATCAATCTCCATTGGTTGCTCTTCATC |
| | TAATTGAGAATATTTATTCTCAGTTAGTGACTTAGATAAATTTTTAATTATGAGGTTTATGATGTAA |
| | TCAAGATTCCAAATGGAAACTTTAAAAGTCCTCATAATAATTAGTAATATCTCTGCTATAGTAACCT |
| | GAAAGTCAACGAGATGAAACATCTGTTGTCACTTACTGTACAAGCAAAGCAATATTGTCACTGCTAC |
| | TGGAATGGTCTGTGTTTAATTTATAGTTGCCAATCCTGTAGCGACTGTATGCAGCAAAACCTGAGTC |
| | ACCTGCTACACGCTGCGAAGCTCCCAATTTGTAATAAGAAAGCGTTCGTGATGTAGCAACAGTGATT |
| | TCTTTAGGCAGGTCCTTGATGTCACAGCGTCCTAGATGGTGTCCAGCAATACGAAGATGTCCACGAA |
| | GGATCACAGCTCCGATTACGAGTTCACTTTCTAGAAGCGGTCTGGTCAGAATAGTGCCATGGAGTGG |
| | CACGTTGAGAAGAATGTTAGTTTCTGGATTGAATGACCACATGGAACGCGTACGCGCAAACAGTCTG |
| | AAAGAAGCAATGAAGTAGCTGAGCCACATCAAGCCTACAAGACAAGCCATTGCGATAGCAATTCCAC |
| | CGGTGATCCAATTTATTCTGTAAACAGCAGCAAGCACAAAACAAGCTAAAGTTACTGGCCATAACAG |
| | CCAGAGGAAAATTAACTTAATTATATACAAAAACCTATTCCTGTTGGCATAGGCAAATTGTAGAAGA |
| | CAAATCCATGTAAGGAATAGGAAACCTATTACTAGGTTCCATTGTTCAAGGAGCTTTTTAAGCTCTT |
| | CAACGGTAATAGTACCGTTGGAATCTGCCATGGCTAAAATTAAAGTTCCAAACAGAAAAACTAATAT |
| | AATATTTAGTTCGTTTAGACCAGAAGATCAGGAACTCTAGAAGAATTCAGATTTTTAACACGAGAGT |
| | AAACGTAAAAAGAAGGTTTTACAAGACTCACGTTAACAATATTGCAGCAGTACGCACACAATCGAAG |
| | CGCAGTAAGGATGGCTAGTGTAACTAGCAAGAATACCACGAAAGCAAGAAAAAGAAGTACGCTATTA |
| | ACTATTAACGTACCTGTCTCTTCCGAAACGAATGAGTACATAAGTTCGTACTCATCAGCTTGTGCTT |
| | ACAAAGGCACGCTAGTAGTCGTCGTCGGTTCATCATAAATTGGTTCCATTACTGGATTAACAACTCC |
| | GGATGAACCGTCGATTGTGTGAATTTGGACATGTTCTTCAGGCTCATCAACAATTTTATTGTAGATG |
| | AAGAAGGTAACATGTTCAACACCAGTGTCTGTACTCAATTGAGTTGAGTACAGCTGGTAATAGTCTG |
| | AAGTGAAGTAACTGTGTAATACAACACAGTCTTTTACTCCAGATTCCCATTTTTCAGTATAACCACC |
| | AATCTGGTAGTCATGTTCAGAAATAGGACTTGTTGTGCCATCACCTGAAGTAATGACAATTGAAGAA |
| | GTTACACTATTGTAAGGTATACAATAGTCGTAACAATTAGTATGCCAGCAAAGAAAATAGTTGGCAT |
| | CATAAAGTAATGGGTTTTTGGAACGGCATTTCCAGCAAAGCCAAAGCCTCATTATTATTCTTACAAA |
| | GTTTATACTCTGCAAGAAGTAGACTAAAGCATAAAGATAGAGAAAAGGGGCTTCAAGGCCAGCAGCA |
| | ACGAGCAAAAGGTGTGAGTAAACTGTTACAAACAACAACAGCAAGTTGCAAACAAAGTGAACACCCT |
| | TGGAGAGTGCTAGTTGCCATCTCTTTTTGAGGGTTATGATTTTGGAAGCGCTCTGAAAAACAGCAAG |
| | AAGTGCAACGCCAACAATAAGCCATCCGAAAGGGAGTGAGGCTTGTATCGGTATCGTTGCAGTAGCG |
| | CGAACAAAATCTGAAGGAGTAGCATCCTTGATTTCACCTTGCTTCAAAGTTACAGTTCCAATTGTGA |
| | AGATTCTCATAAACAAATCCATAAGTTCGTTTATGTGTAATGTAATTTGACTCCTTTGAGCACTGGC |
| | TCAGAGTCGTCTTCATCAAATTTGCAGCAGGATCCACAAGAACAACAGCCCTTGAGACAACTACAGC |
| | AACTGGTCATACAGCAAAGCATAATTGTCACCATTACTATGGCAATCAAGCCAGCTATAAAACCTAG |
| | CCAAATGTACCATGGCCATTTTATATACTGCTCATACTTTCCAAGTTCTTGGAGATCGATGAGAGAT |
| | TCATTTAAATTCTTGGCAACCTCATTGAGGCGGTCAATTTCTTTTTGAATGTTTACAACTGAAGCAT |
| | TAATGCCAGAGATGTCACCTAAATCAACATCTGGTGATGTATGATTCTTAAAATATTTATCTAACTC |
| | CTCCTTGAATGAGTCTAATTCAGGTTGCAAAGGATCATAAACTGTGTTGTTGACAATTCCTATTACA |
| | ACATCACAGTTAGCAGACACAAATGTGTTGTCTGTAGTAATGATTTGTGGTTCATAAAAATTCCTTT |
| | GTGTTACAAACCAGTGTGTGCCATTTGAAACAAAGACACCTTCACGAGGAAAGTGTGCTTTTCCATC |
| | ATGACAAATGGCAGGAGCAGTTGTGAAGTTCTTTTCTTGTGCAGGGACATAAGTCACATGCAAGAAG |
| | ACTACACCATGAGGTGCTGACTGAGGGAAGGACATAAGATGATAGCCCTTTCCACAAAAATCAACTC |
| | TTTTTGATTGTCCAAGTACACACTCTGACATTTTAGTAGCAGCAAGATTAGCAGAAGCTCTGATTTC |
| | TGCAGCTCTAATTAATTGTTGAGTCACATATGTCTGCAAACTTTGAAGTCTGCCTGTGATCAACCTA |
| | TCAATTTGCACTTCAGCCTCAACTTTGTCAAGACGTGAAAGGATATCATTTAAAACACTTGAAATTG |
| | CACCAAAATTGGAGCTAAGTTGTTTAACAAGCGTGTTTAAAGCTTGTGCATTTTGGTTGACCACATC |
| | TTGAAGTTTTCCAAGTGCACTTGCTGTGGAAGAAAGTGAGTCTTGAATTTTGCCAATAGCACTATTA |
| | AATTGGTTGGCAATCAATTTTTGGTTCTCATAGAGAACATTCTGTGTAACTCCAATACCATTAAACC |
| | TATAAGCCATTTGCATAGCAAATGGTATTTGTAATGCAGCACCTGCACCAAAGGTCCAACCAGAAGT |
| | GATTGTACCCGCTAACAGTGCAGAAGTGTATTGAGCAATCATTTCATCTGTGAGCAAAGGTGGCAAA |
| | ACAGTAAGGCCGTTAAACTTTTGTGCACAAATGAGGTCTCTAGCAGCAATATCACCAAGGCAATCAC |
| | CATATTGTTTGATGAAGCCAGCATCTGCAAGTGTCACTTTGTTGAAAAGTAGATCTTCAATAAATGA |
| | CCTCTTGCTTGGTTTTGATGGATCTGGTAATATTTGTGAAAAATTAAAACCACCAAAATCTTTAATT |
| | GGTGGTGTTTTGTAAATTTGTTTGACTTGTGCAAAAACTTCTTGGGTGTTTTTGTCTTGTTCAACAG |
| | CTATTCCAGTTAAAGCACGGTTTAATTGTGTACAAAAACTGCCATATTGCAACAAAAGATTGCTGCA |
| | TTCAGTTGAATCACCACAAATGTACATTGTACAATCTACTGATGTCTTGGTCATAGACACTGGTAGA |
| | ATTTCTGTGGTAACACTAATAGTAAAATTTGTGGGTATGGCAATAGAGTTATTAGAGTAAGCAACTG |
| | AATTTTCTGCACCAAGTGACATAGTGTAGGCAATGATGGATTGACTAGCTACACTACGTGCCCGCCG |
| | AGGAGAATTAGTCTGAGTCTGATAACTAGCGCATATACCTGCACCAATGGGTATGTCACACTCATAT |
| | GAGTTGTTGACATGTTCAGCCCCTATTAAACAGCCTGCACGTGTTTGAAAAACATTAGAACCTGTAG |
| | AATAAACACGCCAAGTAGGAGTAAGTTGATCTGCATGAATAGCAACAGGGACTTCTGTGCAGTTAAC |
| | ATCCTGATAAAGAACAGCAACCTGGTTAGAAGTATTTGTTCCTGGTGTTATAACACTGACACCACCA |
| | AAAGAACATGGTGTAATGTCAAGAATCTCAAGTGTCTGTGGATCACGGACAGCATCAGTAGTGTCAG |
| | CAATGTCTCTGCCAAATTGTTGGAAAGGCAGAAACTTTTTGTTAGACTCAGTAAGAACACCTGTGCC |
| | TGTTAAACCATTGAAGTTGAAATTGACACATTTGTTTTTAACCAAATTAGTAGACTTTTTAGGTCCA |
| | CAAACAGTTGCTGGTGCATGTAGAAGTTCAAAAGAAAGTACTACTACTCTGTATGGTTGGTAACCAA |
| | CACCATTAGTGGGTTGGAAACCATATGATTGTAAAGGAAAGTAACAATTAAAACCTTCAACACCATT |
| | ACAAGGTGTGCTACCGGCCTGATAGATTTCAGTTGAAATATCTCTCTCAAAAGGTTTGAGATTAGAC |
| | TTCCTAAACAATCTATACAGGTAATTATAATTACCACCAACCTTAGAATCAAGATTGTTAGAATTCC |
| | AAGCTATAACGCAGCCTGTAAAATCATCTGGTAATTTATAATTATAATCAGCAATCTTTCCAGTTTG |
| | CCCTGGAGCGATTTGTCTGACTTCATCACCTCTAATTACAAATGAATCTGCATAGACATTAGTAAAG |
| | CAGAGATCATTTAATTTAGTAGGAGACACTCCATAACACTTAAAAGTGGAAAATGATGCGGAATTAT |
| | ATAGGACAGAATAATCAGCAACACAGTTGCTGATTCTCTTCCTGTTCCAAGCATAAACAGATGCAAA |
| | TCTGGTGGCGTTAAAAACTTCACCAAAAGGGCACAAGTTTGTAATATTAGGAAATCTAACAATAGAT |
| | TCTGTTGGTTGGACTCTAAAGTTAGAAGTTTGATAGATTCCTTTTTCTACAGTGAAGGATTTCAACG |
| | TACACTTTGTTTCTGAGAGAGGGTCAAGTGCACAGTCTACAGCATCTGTAATGGTTCCATTTTCATT |
| | ATATTTTAATAGAAAAGTCCTAGGTTGAAGATAACCCACATAATAAGCTGCAGCACCAGCTGTCCAA |
| | CCTGAAGAAGAATCACCAGGAGTCAAATAACTTCTATGTAAAGCAAGTAAAGTTTGAAACCTAGTGA |
| | TGTTAATACCTATTGGCAAATCTACCAATGGTTCTAAAGCCGAAAAACCCTGAGGGAGATCACGCAC |
| | TAAATTAATAGGCGTGTGCTTAGAATATATTTTAAAATAACCATCAATATTCTTAAACACAAATTCC |
| | CTAAGATTTTTGAAATTACCCTGTTTTCCTTCAAGGTCCATAAGAAAAGGCTGAGAGACATATTCAA |
| | AAGTGCAATTATTCGCACTAGAATAAACTCTGAACTCACTTTCCATCCAACTTTTGTTGTTTTTGTG |
| | GTAATAAACACCCAAAAATGGATCATTACAAAATTGAAATTCACAGACTTTAATAACAACATTAGTA |
| | GCGTTATTAACAATAAGTAGGGACTGGGTCTTCGAATCTAAAGTAGTACCAAAAATCCAGCCTCTTA |
| | TTATGTTAGACTTCTCAGTGGAAGCAAAATAAACACCATCATTAAATGGTAGGACAGGGTTATCAAA |
| | CCTCTTAGTACCATTGGTCCCAGAGACATGTATAGCATGGAACCAAGTAACATTGGAAAAGAAAGGT |
| | AAGAACAAGTCCTGAGTTGAATGTAAAACTGAGGATCTGAAAACTTTGTCAGGGTAATAAACACCAC |
| | GTGTGAAAGAATTAGTGTATGCAGGGGGTAATTGAGTTCTGGTTGTAAGATTAACACACTGACTAGA |
| | GACTAGTGGCAATAAAACAAGAAAAACAAACATTGTTCGTTTAGTTGTTAACAAGAACATGAGTAGA |
| | AATAACAACTCTGTTGTTTTCTCTAATTATAAGTCTACCTTTACTAAGAAGAGATAAAATCATATCA |
| | TTGATTTGACCTTCTTTTAAAGACATAACAGCAGTACCCCTTAATTTAAGGGGAAATTTACTCATGT |
| | CAAATAAAGAATAGGAAGACAACTGAATTGGATTTGTATTCCTCCAAAATATGTAATTTGCATGCAT |
| | GACATAACCATCTATTTGTTCGCGTGGTTTGCCAAGATAATTACATCCAATTAAAAATGCTTCAGAT |
| | GATGACGCATTCACATTAGTAACAAAGGCTGTCCACCATGCGAAGTGTCCCATGAGCTTATAAAGAT |
| | CAGCATTCCAAGAATGTTCTGTTATCTTTATAGCCACGGAACCTCCAAGAGCTAGCTTTTGTTGTAT |
| | AAACCCACAAATGTAAGTGAAAAAACCCTCTTTAGAGTCATTTTCTTTTGTAACATTTTTAGTCTTA |
| | GGGTCGTACATATCACTAATAATGAGATCCCATTTATTAGCTGTATGTACAGTTGCACAATCACCAA |
| | TCAAAGTTGAATCTGCATCAGAGACAAAGTCATTAAGATCTGAATCGACAAGCAGCGTACCCGTAGG |
| | CAACCACTGTCTTAAAACAGCTGTACCTGGTGCAACTCCTTTATCAGAACCAGCACCAAAATGTATA |
| | ACTCTCATATTATAGGGTACAGCTAATGTTAATGTGTTTAAATATTGACACAGTTGAGTATATTTTG |
| | CGACATTCATCATTATGCCTTTAGGTAATGTTGCACTATCACCATAATTTTGAAGGTCACACTTTTC |
| | TAATAGCATTCTTTGCATTTTGTAAAGATTAGGCATAGCAACACCCGGTTGCCACGCTTGACTAGAT |
| | TGTAATTTTGGGTAAAATGTTTCTACATGGCCATCTTTACACCAAAGCATAAATGAAATTTCTGTAT |
| | AGTCAATAGTCACTTTGACAACCTTAGAAACTACAGATAAATCTTGGGATTTTATTATTTCAACAAA |
| | ATCATCAAGTAATAAATCAATAACAGAACACACACACTTAGATGAACCTGTTTGCGCATCTGTTATG |
| | AAATAGTTTTTAACTGTACTGTCCATAGGAATAAAATCTTCTAATTCAAAAGGTGATTCCTTAAAAC |
| | GTTTAGCTAGTCCAATCAGTAGATGTAAACCACCTAACTGACTATGACTAAAATCTCCATAAACGAT |
| | ATGTTCGAAGGCATAGCCTTCTAATTTATACCGTTCAATGAATTCATCCATAGCTAATTCTAAGAAA |
| | TCAATTTCCATTTGACTCCTGGGTTTAAATTCTTGTAAATTTCTACTCTGAGTAAAGTAAGTTTCAG |
| | GTAATTGTTGGACAACACCATCAACTTTCTTATAATAATTGAACTGTGTTTTTACGGCTTCTCCAAT |
| | TAATGTGACTCCATTAAGACTAGCTTGTTTGGGACCTACAGATGGTTGTAAACCTTTAACACTACCT |
| | TCTGTAATAAGAACACCATTACGGGCATTTCTAAATAAGTCTACTTGACCATCAACTCTACCATCAA |
| | AAAAGACAGTGAGTGGTGCACAAATCGTTTCAGTTGGTTTCTTGGCTATGTCAGTCATAGAACAAAC |
| | ACCAATAGTAGATATATGTGCTGGAGCATCTCTTTTGTAGTCCCAGATCACAGTATTAGCAGCAATG |
| | TCCACACCCAAATTATTGAGTATTTTCACCTCTGGTACTGGTTTAATGTTGCGCTTAGCCCAAAGCT |
| | CAAATGCTACATTAACAGGTAATGTTGTTTTATTTTCAAACAATTCTACATCAACACCATCAACTTT |
| | TGTGTAAACAGTGTTATTAATGATAGAAACTGGTACTTCACCCTGTTGTCCATCAAAGTGTCCCTTA |
| | TTTACAACATTAAAAGCCACATTTTCTAAACTCTGAAGTCTTGTAAAAGTGTTCCAGAGGTTATAAG |
| | TATCAAATTGTTTGTAAACCCACAAGCTAAAGCCAGCTGAGATCATCATGTTATAAGCATCGAGATA |
| | CAATCTGTACTCATTAGCATGATGTCTACAGACAGCACCACCTAAATTGCAACGTGTTATACACGTA |
| | GCAGACTTTAGTGGTACATAATCTATATCTGACACTACTTGTTTTCCATGAGACTCACATGGACTGT |
| | CAGAGTAATAGAAAAATGGTAATTGTTTTAAATTAACAAAAGCACTTTTATCAAAAGCTGGTGTGTG |
| | GAATGCATGTTTATTTACATACAAACTGCCACCATCACAACCAGGCAAGTTAAGGTTAGATAGCACT |
| | CTAGTGTCAAATCTACAAACAATGGAATTAGCAGGATATCTATCGACATTGCAATTCCAAAATAGGC |
| | ATACACCATCTGTGAATTTGTCAGAATGTGTGGCATAAGAATAGAATAATTCTTCTATTTTATAAGC |
| | TTTGTCACTACAAGGCTGTGCATCATAGAACTTCCATTCTACATCAGCTTGAGGTACACACTTAATA |
| | GCTTTAGGGTTACCAATGTCGTGAAGAACTGGGAATTTGTCTGCTAATAATGCAGCTTTAACAACCA |
| | TGTGTTGAACCTTTCTACAAGCCGCATTAATCTTCAGTTCATCACCAATTATAGGATATTCAATAGT |
| | CCAGTCAACACGCTTAACAAAGCACTCGTGGACAGCTAGACACCTAGTCATGATTGCATCACAACTA |
| | GCTACATGTGCATTACCATGGACTTGACAATACAGATCATGGTTGCTTTGTAGGTTACCTGTAAAAC |
| | CCCATTGTTGAACATCAATCATAAACGGATTATAGACGTAATCAAATCCAATAGAATGATGCCAACA |
| | GGCATAAGTGTCTGAAGCAGTGGAAAAGCATGTGGCACGTCTATCACATAGACAACAGGTGCGCTCA |
| | GGTCCTATTTTCACAAAATACTTCATAGATGTCAACTCAAAGCCATGTGCCCATAAGACAAATACGA |
| | CTCTGTCAGAGAGATTTTTAAGTGTGTCACTTAACATTTGTACAATCTTTATACGCACTACATTCCA |
| | AGGAAGTCCTTTGTACATAAGTGGTATGAGGTGTTTAAATTGATCTCCAGGCGGTGGTTTAGCACTA |
| | ACTCTGGAAAAATCTGTATTATTAGGTGTATCAACATAACCTGTAGGTACAGCAACTAGGTTAACAC |
| | CTGTAGAAAAACCTAGCTGTAAAGGTAAATTGGTACCAACAGCTTCTCTAGTAGCATGACACCCCTC |
| | GACATCGAAGCCAATCCATGCACGTACATGTCTTATAGCTTCTTCGCGGGTGATAAACATGTTAGGG |
| | TAACCATTAACTTGATAATTCATTTTAAAACCCATCATAGAGATGAGTCTTCTATAGGTCATGTCCT |
| | TAGGTATGCCAGGTATGTCAACACATAAACCTTCAGTTTTGAATTTAGTGTCAACACTGAGGTGTGT |
| | AGGTGCCTGTGTAGGATGTAACCCAGTGATTACCTTACTACAATCTTTAAAGAGTCCTGTTACATTT |
| | TCAGCTTGTAAAGTTGCCACATTCCTACGTGGAATTTCAAGACTTGTAAATTGCAACTTGTCATAAA |
| | GGTCTCTATCAGACATTATGCAAAGTATGCCTACTTTTGCTCTGGTAATAGCAACATTAAATCTGTT |
| | TACATTACAAGAGTGAGCTGTTTCAGTGGTTTGAGTGAATATGACATAGTCATATTCTGAGCCCTGT |
| | GATGAATCAACAGTTTGAGTTGGTAGTCCCAAAATCTTTGAGGCTACAGCATTCTGTGAATTATAAG |
| | GTGAAATAAAGACAGCTTTTCTCCAAGCAGGGTTACGTGTAAGGAATTCTCTTACCACGCCTATTTG |
| | TGGCCTGTTAATTGCAGATGAAACATCATGCGTGATAACACCCTTATAAAACATTTTAAAGCATTGA |
| | GCTGATTTGTCTTTATGTGCTTTAAGCTTATTATCATAAACCAAAGCACTCACAGTGTCAACAATTT |
| | CAGCAGGACAACGCCGACAAGTTCCGAGGAACATGTCTGGACCTATAGTTTTCATAAGTCTACACAC |
| | TGAATTGAAATATTCTGGTTCTAGTGTGCCCTTAGTTAGCAATGTGCGTGGTGCAGGTAATTGAGCA |
| | GGGTCGCCAATGTACACATAGTGCTTAGCACGTAATCTGGCATTGACAACACTCAAATCATAATTTG |
| | TGGCCATTGAAATTTCATCAAAGACAACTATATCTGCTGTCGTCTCAGGCAATGCATTTACAGTACA |
| | AAAGACATACTGTTCTAATGTTGAATTCACTTTGAATTTATCAAAACACTCTACACGAGCACGTGCA |
| | GGTATAATTCTACTACATTTATCTATAGGCAAATATTTTAATGCCTTCTCACATAGTGCATCAACAG |
| | CGGCATGAGAGCAAGCTGTATACACTATGCGAGCAGAAGGGTAGTAGAGAGCTAGGCCAATAGCAAA |
| | ATGACTCTTACCAGTACCAGGTGGTCCCTGGAGTGTAGAATACTTTTGCATACCAACCTTTTGATAA |
| | TTTGCAACATTGCTAGAAAACTCATCTGAGATATTGAGTGTTGGGTATAAGCCAGTAATTCTAACAT |
| | AGTGCTCTTGTGGCACTAGTGTAGGTGCACTTAATGGCATTACTGTATGTGATGTCAGCACAAAATA |
| | ATCACCAACATTTAATTTGTAAGTTGTTGTACCTCGGTAAACAACAGCATCACCATAGTCACCTTTT |
| | TCAAAGGTGTACTCTCCTATTTGTACTTTACTGTTTTTAGTTACACGATAACCAGTAAAGACATAAT |
| | TTCGGTTAAGTGGTGGTCTAGGTTTACCAACTTCCCATGAAAGATGTAATTCTCTGTCAGACAGCAC |
| | TTCACGTACAGTAGCAATACCATAAGACAGTTTAAATGTCTCCTCAGTAGCTTTGAGCGTTTCTGCT |
| | GCAAAAAGCTTGAGTCTTTCAGTACAGGTGTTAGCTAAAATGTAATCACCAGCATTTGTCCAGTCAC |
| | ATGTTGCAATTGCATTAAAGTCAGTAACATTATCGCTACCAACACATGTATTTTTATATAAACCAAA |
| | AACTTGTCCATTAGCACACAATGGAAAACTAATGGGTGGTTTATGTGATTTACAATAATAGCTCATA |
| | CCTCCTAAGTAAAGTTGAGTCACATCTGTGACATCACAACCTGGAGCATTGCAAACATACGGATTAA |
| | CAGACAAGACTAATTTATGTGATGTTGATATGACATGGTCGTAACAGCATTTACAACATAAGAATGG |
| | TCTACGTATGCAAGCACCACATCTTAATGAAGTCTGTGAATTGCAAAGAACACAAGCCCCAACAGCC |
| | TGTAAGACTGTATGCGGTGTGTACATAGCCTCATAAAACTCAGGTTCCCAATACCTTGAAGTGTTAT |
| | CATTAGTAAGCATAACAGAATACATGTCTAACATGTGTCCTGTTAACTCATCATGTAGCTTTCTTAT |
| | GTATTGTAAGTACAAATGAAAGACATCAGCATACTCCTGATTAGGATGTTTAGTAAGTGGGTAAGCA |
| | TCTATAGCTAAAGACACGAACCGTTCAATCATAAGTGTACCATCTGTTTTTACGATATCATCTACAA |
| | AACAGCCGGCCCCTAGGATTCTTGATGGATCTGGGTAAGGAAGGTACACATAATCATCACCCTGTTT |
| | AACTAGCATTGTATGTTGAGAGCAAAATTCATGAGGTCCTTTAGTAAGGTCAGTCTCAGTCCAACAT |
| | TTTGCTTCAGACATAAAAACATTGTTTTGATAATAAAGAACTGACTTAAAGTTCTTTATGCTAGCCA |
| | CTAGACCTTGAGATGCATAAGTGCTATTGAAACACACAACAGCATCGTCAGAGAGTATCATCATTGA |
| | GAAATGTTTACGCAAATATGCGTAAAACTCATTCACAAAGTCTGTGTCAACATCTCTATTTCTATAG |
| | AGACACTCATAAAGTCTGTGTTGTAAATTGCGGACATACTTATCGGCAATTTTGTTACCATCAGTAG |
| | ATAAAAGTGCATTAACATTGGCCGTGACAGCTTGACAAATGTTAAAAACACTATTAGCATAAGCAGT |
| | TGTGGCATCTCCTGATGAGGTTCCACCTGGTTTAACATATAGTGAACCGCCACACATGACCATTTCA |
| | CTCAATACTTGAGCACACTCATTAGCTAATCTATAGAAACGGTGTGACAAGCTACAACACGTTGTAT |
| | GTTTGCGAGCAAGAACAAGTGAGGCCATAATTCTAAGCATGTTAGGCATGGCTCTATCACATTTAGG |
| | ATAATCCCAACCCATAAGGTGAGGGTTTTCTACATCACTATAAACAGTTTTTAACATGTTGTGCCAA |
| | CCACCATAGAATTTGCTTGTTCCAATTACTACAGTAGCTCCTCTAGTGGCGGCTATTGATTTCAATA |
| | ATTTTTGATGAAACTGTCTATTGGTCATAGTACTACAGATAGAGACACCAGCTACGGTGCGAGCTCT |
| | ATTCTTTGCACTAATGGCATACTTAAGATTCATTTGAGTTATAGTAGGGATGACATTACGTTTTGTA |
| | TATGCGAAAAGTGCATCTTGATCCTCATAACTCATTGAATCATAATAAAGTCTAGCCTTACCCCATT |
| | TATTAAATGGAAAACCAGCTGATTTGTCTAGGTTGTTGACGATGACTTGGTTAGCATTAATACAGCC |
| | ACCATCGTAACAATCAAAGTACTTATCAACAACTTCAACTACAAATAGTAGTTGTCTGATATCACAC |
| | ATTGTTGGTAGATTATAACGATAGTAGTCATAATCGCTGATAGCAGCATTACCATCCTGAGCAAAGA |
| | AGAAGTGTTTTAATTCAACAGAACTTCCTTCCTTAAAGAAACCCTTAGACACAGCAAAGTCATAGAA |
| | GTCTTTGTTAAAATTACCGGGTTTGACAGTTTGAAAAGCAACATTGTTAGTAAGTGCAGCTACTGAA |
| | AAGCACGTAGTGCGTTTATCTAGTAATAGATTACCAGAAGCAGCGTGCATAGCAGGGTCAGCAGCAT |
| | ACACAAGTAATTCCTTAAAACTAAGTCTAGAGCTATGTAAGTTTACATCCTGATTATGTACAACACC |
| | TAGCTCTCTGAAGTGGTATCCAGTTGAAACTACAAATGGAACACCATCAACAAATATTTTTCTCACT |
| | AGTGGTCCAAAACTTGTAGGTGGGAACACTGTAGAGAATAAAACATTAAAGTTTGCACAATGCAGAA |
| | TGCATCTGTCATCCAAACAGTTAACACAATTTGGGTGGTATGTCTGATCCCAATATTTAAAATAACG |
| | GTCAAAGAGTTTTAACCTCTCTTCCGTGAAGTCATATTTTAACAAATCCCACTTAATGTAAGGCTTT |
| | GTTAAGTCAGTGTCAACATGTGACTCTGCAGTTAAAGCCCTGGTCAAGGTTAATATAGGCATTAACA |
| | ATGAATAATAAGAATCTACAACAGGAACTCCACTACCTGGCGTGGTTTGTATGAAATCACCGAAATC |
| | ATACCAGTTACCATTGAGATCTTGATTATCTAATGTCAGTACACCAACAATACCAGCATTTCGCATG |
| | GCATCACAGAATTGTACTGTTTTTAACAAAGCTTGGCGTACACGTTCACCTAAGTTGGCGTATACGC |
| | GTAATATATCTGGGTTTTCTACAAAATCATACCAGTCCTTTTTATTGAAATAATCATCATCACAACA |
| | ATTGTATGTGACAAGTATTTCTTTTAATGTGTCACAATTACCTTCATCAAAATGCCTTAAAGCATAG |
| | ACGAGGTCTGCCATTGTGTATTTAGTAAGACGTTGACGTGATATATGTGGTACCATGTCACCGTCTA |
| | TTCTAAACTTAAAGAAGTCATGTTTAGCAACAGCTGGACAATCCTTAAGTAAATTATAAATTGTTTC |
| | TTCATGTTGGTAGTTAGAGAAAGTGTGTCTCTTAACTACAAAGTAAGAATCAATTAAATTGTCATCT |
| | TCGTCCTTTTCTTGGAAGCGACAACAATTAGTTTTTAGGAATTTAGCAAAACCAGCTACTTTATCAT |
| | TGTAGATGTCAAAAGCCCTGTATACGACATCAGTACTAGTGCCTGTGCCGCACGGTGTAAGACGGGC |
| | TGCACTTACACCGCAAACCCGTTTAAAAACGATTGTGCATCAGCTGACTGAAGCATGGGTTCGCGGA |
| | GTTGATCACAACTACAGCCATAACCTTTCCACATACCGCAGACGGTACAGACTGTGTTTTTAAGTGT |
| | AAAACCCACAGGGTCATTAGCACAAGTTGTAGGTATTTGTACATACTTACCTTTTAAGTCACAAAAT |
| | CCTTTAGGATTTGGATGATCTATGTGGCAACGGCAGTACAGACAACACGATGCACCACCAAAGGATT |
| | CTTGATCCATATTGGCTTCCGGTGTAACTGTTATTGCCTGACCAGTACCAGTGTGTGTACACAACAT |
| | CTTAACACAATTAGTGATTGGTTGTCCCCCACTAGCTAGATAATCTTTGTAAGCTTTAGCAGCATCT |
| | ACAGCAAAAGCACAGAAAGATAATACAGTTGAATTGGCAGGCACTTCTGTTGCATTACCAGCTTGTA |
| | GACGTACTGTGGCAGCTAAACTACCAAGTACCATACCTCTATTTAGGTTGTTTAATCCTTTAATAAA |
| | GTATAAATACTTCACTTTAGGACCTTTAGGTGTGTCTGTAACAAACCTACAAGGTGGTTCCAGTTCT |
| | GTATAGATAGTACCAGTTCCATCACTCTTAGGGAATCTAGCCCATTTCAAATCCTGTAAATCGGATA |
| | ACAGTGCAAGTACAAACCTACCTCCCTTTGTTGTGTTGTAGTAAGCTAACGCATTGTCATCAGTGCA |
| | AGCAGTTTGTGTAGTACCGGCAGCACAAGACATCTGTCGTAGTGCAACAGGACTAAGCTCATTATTC |
| | TGTAATTTGACAGCAGAATTGGCCCTTAAAGCTGTTACAATAAGAGGCCATGCTAAATTAGGTGAAT |
| | TGTCCATACTAATTTCACTAAGTTGAACAATTTTACTATCTGCATCTACAACCTGTTGGATTTCCCA |
| | CAATGCTGATGCATAAGTAAATGTTGTACCATCACACGTATTTTTATATGTGTTATAGTCTGGTATG |
| | ACAACCATTAGTTTGGCTGCTGTTGTAAGAGGTATTATGTTCAAGGGAACACAACCATCTCTTGCAT |
| | TGTTGATAATGTTGTTGAGTGCATCATTATCCAACTTTCTAAGCATAGTGAAAAGCATTGTCTGCAT |
| | AGCACTAGTAACTTTTGCCCTCTTGTCCTCAGATCTAGCCTGTTTATACATTTGGGTCATAGCTTGA |
| | TCAGCCATCTTTTCCAACTTACGTTGCATGGCTGCATCACGGTCAAATTCAGATTTAGCCACATTCA |
| | AAGACTTCTTCAACTTTTTAAGAACAACTTCAGAATCACCATTAGCAACAGCCTGCTCATAAGCTTC |
| | TTGAGCAGTAGCAAAAGCTGCATATGATGGAAGGGAACTAAACTCTGAGGCTATAGCTTGTAAGGTT |
| | GCCCTGTTGTCCAGCATTTCTTCACAAAGCTTGTTTATGTCTACAGCACCCTGCATGGAAAGCAAAA |
| | CAGAAAGTAGTGAAACCATTTTTTCAAAGGCTTCAGTAGTATCTTTAGCTAAGAGAATGTCATTGTG |
| | TAACTGGACACATTGAGCCCACAATTTAGATGATGATTCTACTCTGAGTTGTTGCAAAACTGAGAGT |
| | AAGACTACTGATGTGCACTTTACATCTGACATTTTAGACTGTACAGTGGCTACTTTGATACAAGGTT |
| | TGCCACCAACACCCAACAATTTAATGTTGAGTTTGAAGGCATCTATGCTATTCTTGGGTGGGAGTAG |
| | TCCCTGTGAATTCATATATCTAAACTCCTGTGTAGAAACTAAGTAATCATAAACACCAAGAGTCAGT |
| | CTAAAGTAGCGGTTGAGTAAACAAAAGAGGCCAAAGTAACAAGTACAAAAATAGCCTAAGAAACAAT |
| | AAACTAGCATTATACACTGAAGTGTATTACCAGTTATGAAGAAAATAGGGCAATACTCAACACACAT |
| | AAAAACAATACCTCTGGCCAAAAACATGACAGTTGTAACTACACCTGAGTAGTTAGAAGTAACAGAG |
| | ATTATAAGAGCCCACATGGAAATGGCTTGATCTAAAGCATTACCATAATAAACTTTATAAACGAGTG |
| | TCAAGACATTCATAAGTGTCCACACTCTCCTAGCACCATCATCATACACAGTTCTTGCTGTCATAAG |
| | GATTAGTAACACTACAGCTGATGCATACATAACACAGTCTTTTAGCTTAAAACCAGACAAACTAGTA |
| | TCAACCATATCCAACCATGTCATAATACGCATCACCCAACTAGCAGGCATATAGACCATATTAAAAT |
| | AAGCTACAGTGGCAAGAGAAGGTAACAAAAACAAACAGAGAAATGCATGCTTATGTTTGACAAACAT |
| | CATTGCAAAAGCAGACATAGCAATAATACCCATAGCAAAAGGTAAAAAGGCATTTTCATACAAAAAA |
| | AAGAACAAAGACCATTGAGTACTCTGGACTAAAACTAAAAGTGAAGTCAAAATTGTGAGTAACAACC |
| | AGTGGTGTGTACCCTTGATTGTTCTTTTCACTGCACTTTGGAAAGTAACACCTGAGCATTGTCTAAC |
| | AACATCAAAAGGTGTAAATTCATCTTCTAATAAAGCACTACCCAATATGGTACGTCCATTCATACCA |
| | TTTTGCAGTAATTCTTTTAATGAAGCACACATATCTAAAACGGCAATTCCAGTTTGAGCAGAAAGAG |
| | GTCCTAGTATGTCAACATGGTCTTGTGTTAGAGGTTCATAATTGTACTTCATAGCCACAAGGTTAAA |
| | GTCATTAAGAGTTGTGGTAAATCGATTGAGAAACCACCTGTCTCCATTTATAACAGCAGCGTACAAC |
| | CAAGCTAAAACATTAACTGTAATAGTTGTGTCCGTACCAGCTGCTTGTGCTGTTTGCCTGTCAACAA |
| | AAGGTCCATAAAAGTTACCTTCTAAGTCTGTGCCAGCATGAACTCCAGTTGGTAATTCCATATGGTG |
| | CATGTAACAAAAAGAGACACAGTCATAATCTATGTTAAAACCAACACTACCACATGAACCATTAAGG |
| | AATGAACCCTTAATAGTGAAATTGGGCCTCATAGCACATTGGTAAACACCAGATGGTGAACCATTGT |
| | AACAAGCTAACACTGAAAAAGTCTGTCCTGGTTGAATGCGAACAAACTTATACTTAGGTGTCTTAGG |
| | ATTGGCTGTATCAACCTTAAGCTTAAGTACACAATTTTGCATAGAATGTCCAATAACCCTGAGTTGA |
| | ACATTACCAGCCTGTACCAAGAAATTATGATTAGACTTACGAATGAGTAAATCTTCATAATTAGGGT |
| | TAAGCATGTCTTCAGAGGTGCAGATCACATGTCTTGGACAGTAAACTACGTCATCAAGCCAAAGACC |
| | GTTAAGTGTAGTTGTACCACAAGTTACTTGTACCATACAACCCTCAACTTTACCAGATGGGAATGCC |
| | ATTTTTCTAAAACCACTCTGCAAAACAGCTGAGGTGATAGAGGTTTGTGGTGGTTGGTAAAGAACAT |
| | CAGAACCTGAGTTACTGAAGTCATTGAGAGCCTTTGCGAGATGACAACAAGCAGCTTCTCTGTAGCT |
| | AGTTGTATCCATTGCTCCACTAAAATACTTGTACTTATTATAAAGAGCTAAGTATCTATTATATTGC |
| | GTAAGAGGTAATAGCACATCACTACGCAACTTTAGATACATTTCTTTATTTAACAAAAAGGTGCACA |
| | GCGCAGCTTCTTCAAAAGTACTAAAGGAAACACCATTAAAGACTACACGTCTCTTTAGGTAATTACT |
| | AAAGAACCAATAGAAATGCTTTGTGGAAATACAAATGATATAAGCAATTGTTATCCAGAAAGGTACT |
| | AAAGGTGTGAACATAACCATCCACTGAATATGTGCTAAAAAAGAAACATCATTAGTAAGATAAAATG |
| | TCAAGTACAAGTAAATAACAGAATAAACACCAGGTAAGAATGAGTAAACTGGTGTTAAACAGAGTAC |
| | AGTGAATGACATAAGGAATAGTAAAGTATTAAAGGCAACTACATGACTGTATTCACCAAAAGCTCTT |
| | CTAAACCTCATAAAATAGTAGGCAAGGCATGTTACTACGATAGCTACAATACCACCAGCTACTATAG |
| | ATGCTGATATGTCCAAAGCACCAATAGGTTGAATTAGTGGTGTAAACATATTAGTAAGTAAATTTAC |
| | AGCATCTACACCACAGAAAACTCCTGGTAAAGATCTGTAATAATCATTGTTAAGTACCCATCTACCA |
| | CTAGTAGATACACAAACACCAGCTTCTGATCTTTCACAAGTGCCGTGCCTACAGTACTCAGAATCAA |
| | AAGTTGTTACCACTCTAACAGAACCTTCAAGGTAGGTGTTAGGAAATTGAATAATAGAGCCATCCAT |
| | GAGCACATAACGTGTGTCAGGGCGTAAACTTTCATAAGCAACAGAACCTTCTAGTACATTGGTATCA |
| | TAACAATATGGTACTGGCTTACCAGAAGCATCTTTAAAAATTGTACATTCAGCAGCCAAAACACAAG |
| | CTGATGTTGCAAAGTCAGTGTACTCTATAAGTTTTGATGGTGTGTAACAGATGTTACCAACTGCACT |
| | AAAAACTCTAGGTAAGAAATGCAAAAAGTCACCATTAGTTGTGCGTAATATCGTGCCAGGCAAACCA |
| | GGCACGACAAAACCCACTTCTCTTGTTATGACTGCAGCAATCAATGGGCAAGCTTTGTCATTAGTAT |
| | AACTACCACCACGCTGGCTAAACCATGTGTCAAAATCAGCATGTTTGTTAGCAAAACAAGTATCTGT |
| | AGATGCTATGTCACGAGTGACACCACCATCAATAGCCTTGTATCCTATGATTTCACTTGAAAAGTCA |
| | GTATGTTTAGACATGACATGAACAGGTGTTATTAAATAGAAAATAGCAGCAACAAAAAGGAACACAA |
| | GTGTAACTTTAATTAACTGCTTCAACCAATTATTAACAATTTTACCACCCTTAAGTGCTATCTTTGT |
| | TGTTACAACATTAACAACTTGTCTAGTAGTTGCACATGTCAACTTAAAAGGTAAGTTATTCTTTTTA |
| | GCAGCACTACGTATTTGTTTTCGTAGTTGTTCAGACAATGACATGAAATCTTTAACGTTCCATATCA |
| | AAGCAATGTTGTGACTTTTTGCTACCTGCGCATTAATATGACGCGCACTACAGTCAATACAAGCACC |
| | AAGGTCACGGGGTGTCATGTTTTCAACTTTGTTATAGGTGAGCATATAGTTATTACAACTATCGCCA |
| | GTAACTTCTATGTCAGATTGATGTGACAATTTAAGACATTCAACAACATCTTTAGTTTCTACATCTG |
| | AATCAACAAACCCTTGCCGAGCTGCTGAAATAAAAGTAGATAAGACATTGTCTAAGGACACATTCTT |
| | TGCAAGTTCAGCTTCTGCAGTTGCAACTAGTGTTTTGAGTTTTTCCATTGGTACGTTAAAAGTTGAT |
| | GAAAACGTATTAACGTAAGCATCAAACATTTTAACTGCAACTTCCGCACTATCACCAACATCAGACA |
| | CTAATGCCTGATCTAGTAACAGTATAGGTTGACACATAAGCTGACTGTAGTAAACAGACGCTGATTT |
| | TGCAGATGATTCTTCACATTTTGATTTACCATCAAAAACTATAACATTAATAGGCAATGAACCTTTA |
| | GTGTTATTAGCTCTCAGGTTGTCTAAGTTAACAAAATGAGAGAGAGAATGTCTTTCATAAGTCTTTT |
| | GACCAGCTTTATCAAAGTAAAGATGGATGGAACCATTCTTCACTGTAACACTATCAACGATGTAAGA |
| | AGACTGGTCAGTAGGATTTATTGGTCTTTTAAACTGTAGTGACAAGTCTCTCGCAACTTCATCACTA |
| | ATAAATGTACTACCAGCACAGAATGTATCACAATTAACACAATTCCAATTGTGTAGTTTGCAAAAGC |
| | CTTTACCTCCATTAGCATAGACATAAAAGGACCTTCTAACACCATTAACAATAGTTGTACATTCGAC |
| | TCTTGTTGCTCTATTACGTTTGTAACACATCATACAAGTTGATGAATTACAACCGTCTACAACATGC |
| | ACATAACTTTTCCATACATAATAAAATGATGCAAAGAAGATGTACATTCTAACCATAGCTGAAATCG |
| | GGGCCATTTGTACAAGATTAATTATTAACCACATAAGCCAAGAATTACTAATAAAATGTACTGCAAA |
| | ATAGCTGAAAAACAATTGCATGATTGCAGCCAATCCAAGTACATAGAAAAACCTAGTGAAAAGAATA |
| | TATGCCAAAAACCACTCTGCAACTAAGCCAAAAGCAGTTAAATCCCATTTAAAAGATGAAATGGTAA |
| | TTTGTATAGTTTCTAAAGAAGGATAGGTGTCTAAAGAATCTAAACCACTAAGACAAACACTACAAGG |
| | TATAGAACCAGTACAGTAGGTTGCAATAGTGACATTAGTAGAGTTCAAATAGCCTTCTCTGTAACCA |
| | GTACAGTAAGAAGGCATGCCTAAATTAGACATTAAAACACCTAAAGCAGCGGTTGAGTAGATTAAAG |
| | AACCTAGGCAAACACTTAATAGTAAAAACCAAATTATAATATTTATCAGTTTAGAAAAATTAGGTGA |
| | CTTCAAATAATTAAATGAAGCCTCTAGACAAAATTTACCGACACTCTTAACAGTATTCTTTGCTATA |
| | GTAGTCGGCATAGATGCTTTAATTCTAGAATTTGTACTTCTAGTAAAAGTACACAATTGTAGCAATA |
| | AAGTAAAGAAATAAGGCATATAATTAGTACAAACACGGTTTAAACACCGTGTAACTATGTTAGTAGT |
| | TGTACTAACAACTTTGTTAAGAAAAGGCTTAGCATAATTAGCTATAGTATCCCAAGGGACACTATTA |
| | ACAGCAGCTAAACCATGAGTAGCAAGGGTTTTCAAACCTAATACTCTAGATAATTCATTAGGTTTCT |
| | TAATAGTAAGACTAGAATTGTCTACATAAGCAGCCATTAGATCTGTGTGGCCAACCTCTTCTGTAAT |
| | TTTTAAACTATTATTTGCTGGTTTAAGTATAATGTCTCCTACAACTTCGGTAGTTTTCACATTACAC |
| | TCAAGAACGTCTTTCTGTATGGTAGGATTTTCCACTACTTCTTCAGAGACTGGTTTTAGATCTTCGC |
| | AGGCAAGATTATCCATTCCCTGCGCGTCCTCTGACTTCAGTACATCAAACGAATTTGATGTTTCAAC |
| | TGGTTTTGTGCTCCAAAGACAACGTATACACCAGGTATTTGGTTTATACGTGGCTTTATTAGTTGCA |
| | TTGTTAACATGCCAAACAATAGGTTTATGTAACAATTTAGCTCCTTTCTTAAAAGAGGGTGTGTAGT |
| | GTTTATAATCAATAGCCACCACATCACCATTTAAGTCAGGGAAAAATGTAACTTTAAGCTCTCTTGA |
| | AGCAGGTTTCTTATAACCAGTTAACTGGTTTAAATCATCAGCAAATTTGATATTATCACATACAAAC |
| | TTAAAATTATCGAAGCTTGCGTTTGGATATGGTTGGTTTGGTACAAGATCAATTGGTTGCTCTGTGA |
| | AATAAGAATTGTCTTTCTTATAATAATTGTCCAACTTAGGGTCAATTTCTGTACAAACAACACCATC |
| | CAATTTATAAGTAACTGGTTTTATGGTTGTTGTGTAACTGTTTTCTTTGTAGAAAACATCCGTAATA |
| | GGACCTTTGTATTCTGAGGACTTTGTAAGTAAAGCACCGTCTATGCAATACAAAGTTTCTTTAGAAG |
| | TTATATGTTTATAGTGACCACACTGGTAATTACCAGTGTACTCACTAGCACAAGTAAATGTACCATG |
| | CTTAAGTTCATACTGAGCAGGTGGTGCTGACATCATAACAAAAGGTGACTCCTGTTGTACTAGATAT |
| | TTTGTAGCTTGTTTACCACACGTACAAGGTATCTGAACACCTTTCTTAAATTGTTCATAAGAAAGTG |
| | TGCCCATGTACATAACAGCTTCTACACCCTTAAGGGTTGTCTGCTGTTGTCCACAAGTTTTACACAC |
| | CACGTTCAAGACTCTTTTGCAAGAATCTAAATTGGCATGTTGAAACAAGTAACTCATTGTTTCTCTA |
| | ACATCACCTAACTCACCTACTGTCTTATTACAGTAGGCTAAGATAAGTGCACAAAAGTTAGCAGCTT |
| | CACCAGCCCTTGCTCTGTAATAAGCATCTTGTAGAGCAGGTGGATTAAACTTCAACTCTATTTGTTG |
| | GAGTGTTAACAATGCAGTGGCAAGATAACAGTTGTTATCTGCCCATTTAATAGAAGTTAAACCATTA |
| | ACTTGTGGGTATTTCCACTTTTTAGTGTGATTTAATGCTGACATGTACCTACCCAGAAAACTAGGAT |
| | CAGTTGTGTGGTAGTACTCAAAAGCCTCAACACGTAGAGTGTCATCATTAGGTAAAACATAAAATGT |
| | TTTACCTTCATGTGAATTATGAGGTTTTATTTTAGTAACATCAGCTCCATCCAAATAAGTTGGACCA |
| | AACTGTTGTCCATATGTCATTGACATGTCCACAACTTGCGTGTGGAGGTTAATGTTGTCTACTGTTG |
| | TAAACACCTTAATAGTCCTCACTTCTCTCAAAGAAAGAAGTGTCTTAAGATTGTCAAAGGTGATAAC |
| | TTCACCATCTAGGTGGAATGTGGTAGGATTACTAGTGTAATATACACTTTTATCACCTCTCTTAAGA |
| | AATTCTATACCTAGTTGTGTAGATTGTCCAGAATAGGACCAATCTTTATAGGAACCAGCAAGTGAGA |
| | TGGTTTCAATAAAATGTTCTTCAGGTGTTTTAGAAGAAGAAGTAAGATAACCATTATACGCTGTAAC |
| | AGCATCAGGTGAAGAAACAGAAACTGTAGCTGGCACTTTGAGAGATCTCATATACCGAGCAGCTTCT |
| | TCCAAATTTAAGCCATGTGTTACATAGCCAAGTGGCATTGTAACAAGAGTTTCATTTAGATCGTTAA |
| | GTGTGTTGATAAGTGACGCTACAGTTGTTTTACTGGTGTAAAAGTAAAATCTAGCACCATAATCAAC |
| | CACACCCTCTTGTATTTTAATACCCTTATATTTACGCTGTATAGTTGAAACTATGGCTTTAGTTTCC |
| | ACACAGACAGGCATTAATTTGCGTGTTTCTTCTGCATGTGCAAGCATTTCTCGCAAATTCCAAGAAA |
| | CAGTTCCAAGAATTTCTTGCTTCTCATTAGAGATAATAGATGGTAGAATGTAAAAGGCACTTTTACA |
| | CTTTTTAAGCACTGTCTTTGCCTCCTCTACAGTGTAACCATTTAAACCCTGACCCGGGTAAGTGGTT |
| | ATATAATTGTCTGTTGGCACTTTTCTCAAAGCTTTCGCTAGCATTTCAGTAGTGCCACCAGCCTTTT |
| | TAGTAGGTATAACCACAGCAGTTAAAACACCCTCTTGAACAACATCACCCACTATATATGGAGCATC |
| | TTTCTTTAAGAAAGTGATGTCAATGTCACTAACAAGAGTGGCAGAATCTGGATGAAGATTGCCATTA |
| | ATGTCAATATAAAGTAACAAGTTTTCTGTGAGGAACTTAGTTTCTTCCAGAGTTGTTGTAACTTCTT |
| | CAACACAAGCTTTGATTTTCTTATCATCTTGTTTTCTCTGTTCAACTGAAGGTTTACTTTCAGTTAT |
| | AAATGGCTTAACTTCCTCTTTAGGAATCTCAGCGATCTTTTGTTCAACTTGCTTTTCACTCTTCATT |
| | TCCAAAAAGCTTGAAACAAGTTTGTCATAGAGATTTTTATCAAAGACAGCTAAGTAGACATTTGTGC |
| | GAACAGTATCTACACAAACTCTTAAAGAATGTATAGGGTCAGCACCAAAAATACCAGCTGATAATAA |
| | TGGTGCAAGTAGAACTTCGTGCTGATTAAAATTTTCATAAGCACTCTTAAGAAGTTGAATGTCTTCA |
| | CCTTTGTTAACATTTGGGCCGACAACATGAAGACAGTGTTTAGCAAGATTGTGTCCGCTTAAAACAC |
| | AACTACCACCCACTTTAAGTGGTCCATTAGTAGCTATGTAATCATCAGATTCAACTTGCATGGCATT |
| | GTTAGTAGCCTTATTTAAGGCTCCTGCAACACCTCCTCCATGTTTAAGGTAAACATTGGCTGCATTA |
| | ACAACCACTGTTGGTTTTACCTTTTTAGCTTCTTCCACAATGTCTGCATTTTTAATGTATACATTGT |
| | CAGTAAGTTTTAAATAACCACTAAAACTATTCACTTCAATAGTCTGAACAACTGGTGTAAGTTCCAT |
| | CTCTAATTGAGGTTGAACCTCAACAATTGTTTGAATAGTAGTTGTCTGATTGTCCTCACTGCCGTCT |
| | TGTTGACCAACAGTTTGTTGACTATCATCATCTAACCAATCTTCTTCTTGCTCTTCTTCAGGTTGAA |
| | GAGCAGCAGAAGTGGCACCAAATTCCAAAGGTTTACCTTGGTAATCATCTTCAGTACCATACTCATA |
| | TTGAGTTGATGGCTCAAACTCTTCTTCTTCACAATCACCTTCTTCTTCATCCTCATCTGGAGGGTAG |
| | AAAGAACAATACATATGTGAAGCCAATTTAAACTCACCAGACTCATCAAATAAGTAGTATGTAGCCA |
| | TACTCCACTCATCTAAATCAATGCCCAGTGGTGTAAGTAATTCAGATACTGGTTGCAAAGTTTTTAT |
| | GACAGCATCTGCCACAACACAGGCGAACTCATTTACTTCTGTACCGAGTTCAACTGTATAGGCAGAG |
| | CACTTCTCATTAAGTACTTTATCAATCCTTTCATCAAGTTCAAAAGTGATATTCACACTCTTGTAAC |
| | CTTGCACTTCTATCACAGTGTCATCACCAAAAGTAACCTTTGTTGGTGCACCGCCTTTGAGTGTGAA |
| | GGTATTGTTTGTTACCATCATATTAGGTGCAAGGGCACAGTACTTTTCTGTGTCTTTGATTTCGAGC |
| | AACATAAGCCCGTTAATACAAACTGGTGTACCAACCAATGGAGCTTCAACAGCTTCACTAGTAGGTT |
| | GTTCTAATGGTTGTAAATCACCAGTTTTCAAGACAACTTCCTCTGTTAACACTTCTGTGGGAAGTGT |
| | TTCTCCCTCTAAGAAGATAATTTCTTTTGGGGCTTTTAGAGGCATGAGTAGGCCAGTTTCTTCTCTG |
| | GATTTAACACACTTTCTGTACAATCCCTTTGAGTGCGTGACAAATGTTTCACCTAAATTCAAGGCTT |
| | TAAGTTTAGCTCCACCAATAATGATAGAGTCAGCACACAAAGCCAAAAATTTATTTACAAGCTTAAA |
| | GAATGTCTGAACACTCTCCTTAATTTCCTTTGCACAGGTGACAATTTGTCCACCGACAATTTCACAA |
| | GCACAGGTTGAGATAAATTTAACAATTTCCCAACCGTCTCTAAGAAACTCTACACCTTCCTTAAACT |
| | TCTCTTCAAGCCAATCAAGGACGGGTTTGAGTTTTTCATAAACAGTGCCAAAGATGTTAGTTAGCCA |
| | CTGCGAAGTCAACTGAACAACACCACCTGTAATGTAGGCCATTACAACTAGATTGTTAGTAGCCAAA |
| | TCAGATGTGAACATCATAGCATCAATGAGTCTCAGTGAATACTGTGAAATTCCATCTAGTATTGTTA |
| | TAGCGGCCTTCTGTAAAACACGCACAGAATTTTGAGCAGTTTCAAGAGTGCGGGAGAAAATTGATCG |
| | TACAACACGAGCAGCCTCTGATGCAAATGCATAAAGAGGACTCAGTATTGATTTCTGTTCACCAATA |
| | TTCCAGGCACCTTTTTTAGCTTTTCCTTTTGTAACTTTAAAATTACCACAGGATTCAACAATTTGTT |
| | TGAATGCTTTATAATCCAAACCTTTCACAGTTTCCACAAAAGCACTTGTGGAAGCAGAAAAAGATGC |
| | CAAAATAATGGCGATCTCTTCATTAAGTTTAAAGTCACCAACAATATTGATGTTGACTTTCTCTTTT |
| | TGGAGTATTTCAAGAAGGTTGTCATTAAGACCTTCGGAACCTTCTCCAACAACACCTGTATGGTTAC |
| | AACCTATGTTAGCGCTAGCACGTGGAACCCAATAGGCACACTTGTTATGGCAACCAACATAAGAGAA |
| | CACACAGCCTCCAAAGGCAATAGTGCGACCACCCTTACGAAGAATGGTTTTCAAGCCAGATTCATTA |
| | TGGTATTCGGCAAGACTATGCTCAGGTCCTACTTCTGAATTGTGACATGCTGGACAATAAATTTTAA |
| | CAACAGCATTTTGGGGTAAGTAACCACAAGTAGTGGCACCTTCTTTAGTCAAATTCTCAGTGCCACA |
| | AAATTCGCAAGTGGCTTTAACAAAATCGCCCGTCTGCCATGAAGTTTCACCACAATGATCACACTTC |
| | ATGAGAGTTGAAAGGCACATTTGGTTGCATTCATTTGGTGACGCAACTGGATAGACAGATCGAATTC |
| | TACCCATAAAGCCATCAAGCTTTTTCTTTTCAACCCTTGGTTGAATAGTCTTGATTATGGAATTTAA |
| | GGGAAATACAAAATTTGGACATTCCCCATTGAAGGTGTCAAATTTCTTTGCCAATTTAATTTCAAAA |
| | GGTGTCTGCAATTCATAGCTCTTTTCAGAACGTTCCGTGTACCAAGCAATTTCATGCTCATGTTCAC |
| | GGCAGCAGTATACACCCCTCTTAGTGTCAATAAAGTCCAGTTGTTCGGACAAAGTGCATGAAGCTTT |
| | ACCAGCACGTGCTAGAAGGTCTTTAATGCACTCAAGAGGGTAGCCATCAGGGCCACAGAAGTTGTTA |
| | TCGACATAGCGAGTGTATGCCCCTCCGTTAAGCTCACGCATGAGTTCACGGGTAACACCACTGCTAT |
| | GTTTAGTGTTCCAGTTTTCTTGAAAATCTTCATAAGGATCAGTGCCAAGCTCGTCGCCTAAGTCAAA |
| | TGACTTTAGATCGGCGCCGTAACTATGGCCACCAGCTCCTTTATTACCGTTCTTACGAAGAAGAACC |
| | TTGCGGTAAGCCACTGGTATTTCGCCCACATGAGGGACAAGGACACCAAGTGTCTCACCACTACGAC |
| | CGTACTGAATGCCTTCGAGTTCTGCTACCAGCTCAACCATAACATGACCATGAGGTGCAGTTCGAGC |
| | ATCCGAACGTTTGATGAACACATAGGGCTGTTCAAGTTGAGGCAAAACGCCTTTTTCAACTTCTACT |
| | AAGCCACAAGTGCCATCTTTAAGATGTTGACGTGCCTCTGATAAGACCTCCTCCACGGAGTCTCCAA |
| | AGCCACGTACGAGCACGTCGCGAACCTGTAAAACAGGCAAACTGAGTTGGACGTGTGTTTTCTCGTT |
| | GAAACCAGGGACAAGGCTCTCCATCTTACCTTTCGGTCACACCCGGACGAAACCTAGATGTGCTGAT |
| | GATCGGCTGCAACACGGACGAAACCGTAAGCAGCCTGCAGAAGATAGACGAGTTACTCGTGTCCTGT |
| | CAACGACAGTAATTAGTTATTAATTATACTGCGTGAGTGCACTAAGCATGCAGCCGAGTGACAGCCA |
| | CACAGATTTTAAAGTTCGTTTAGAGAACAGATCTACAAGAGATCGAAAGTTGGTTGGTTTGTTACCT |
| | GGGAAGGTATAAACCTTTAAT |
| 2 | GenBank Accession No. AY278741.1 (SARS coronavirus Urbani, complete genome) |
| | ATATTAGGTTTTTACCTACCCAGGAAAAGCCAACCAACCTCGATCTCTTGTAGATCTGTTCTCTAAA |
| | CGAACTTTAAAATCTGTGTAGCTGTCGCTCGGCTGCATGCCTAGTGCACCTACGCAGTATAAACAAT |
| | AATAAATTTTACTGTCGTTGACAAGAAACGAGTAACTCGTCCCTCTTCTGCAGACTGCTTACGGTTT |
| | CGTCCGTGTTGCAGTCGATCATCAGCATACCTAGGTTTCGTCCGGGTGTGACCGAAAGGTAAGATGG |
| | AGAGCCTTGTTCTTGGTGTCAACGAGAAAACACACGTCCAACTCAGTTTGCCTGTCCTTCAGGTTAG |
| | AGACGTGCTAGTGCGTGGCTTCGGGGACTCTGTGGAAGAGGCCCTATCGGAGGCACGTGAACACCTC |
| | AAAAATGGCACTTGTGGTCTAGTAGAGCTGGAAAAAGGCGTACTGCCCCAGCTTGAACAGCCCTATG |
| | TGTTCATTAAACGTTCTGATGCCTTAAGCACCAATCACGGCCACAAGGTCGTTGAGCTGGTTGCAGA |
| | AATGGACGGCATTCAGTACGGTCGTAGCGGTATAACACTGGGAGTACTCGTGCCACATGTGGGCGAA |
| | ACCCCAATTGCATACCGCAATGTTCTTCTTCGTAAGAACGGTAATAAGGGAGCCGGTGGTCATAGCT |
| | ATGGCATCGATCTAAAGTCTTATGACTTAGGTGACGAGCTTGGCACTGATCCCATTGAAGATTATGA |
| | ACAAAACTGGAACACTAAGCATGGCAGTGGTGCACTCCGTGAACTCACTCGTGAGCTCAATGGAGGT |
| | GCAGTCACTCGCTATGTCGACAACAATTTCTGTGGCCCAGATGGGTACCCTCTTGATTGCATCAAAG |
| | ATTTTCTCGCACGCGCGGGCAAGTCAATGTGCACTCTTTCCGAACAACTTGATTACATCGAGTCGAA |
| | GAGAGGTGTCTACTGCTGCCGTGACCATGAGCATGAAATTGCCTGGTTCACTGAGCGCTCTGATAAG |
| | AGCTACGAGCACCAGACACCCTTCGAAATTAAGAGTGCCAAGAAATTTGACACTTTCAAAGGGGAAT |
| | GCCCAAAGTTTGTGTTTCCTCTTAACTCAAAAGTCAAAGTCATTCAACCACGTGTTGAAAAGAAAAA |
| | GACTGAGGGTTTCATGGGGCGTATACGCTCTGTGTACCCTGTTGCATCTCCACAGGAGTGTAACAAT |
| | ATGCACTTGTCTACCTTGATGAAATGTAATCATTGCGATGAAGTTTCATGGCAGACGTGCGACTTTC |
| | TGAAAGCCACTTGTGAACATTGTGGCACTGAAAATTTAGTTATTGAAGGACCTACTACATGTGGGTA |
| | CCTACCTACTAATGCTGTAGTGAAAATGCCATGTCCTGCCTGTCAAGACCCAGAGATTGGACCTGAG |
| | CATAGTGTTGCAGATTATCACAACCACTCAAACATTGAAACTCGACTCCGCAAGGGAGGTAGGACTA |
| | GATGTTTTGGAGGCTGTGTGTTTGCCTATGTTGGCTGCTATAATAAGCGTGCCTACTGGGTTCCTCG |
| | TGCTAGTGCTGATATTGGCTCAGGCCATACTGGCATTACTGGTGACAATGTGGAGACCTTGAATGAG |
| | GATCTCCTTGAGATACTGAGTCGTGAACGTGTTAACATTAACATTGTTGGCGATTTTCATTTGAATG |
| | AAGAGGTTGCCATCATTTTGGCATCTTTCTCTGCTTCTACAAGTGCCTTTATTGACACTATAAAGAG |
| | TCTTGATTACAAGTCTTTCAAAACCATTGTTGAGTCCTGCGGTAACTATAAAGTTACCAAGGGAAAG |
| | CCCGTAAAAGGTGCTTGGAACATTGGACAACAGAGATCAGTTTTAACACCACTGTGTGGTTTTCCCT |
| | CACAGGCTGCTGGTGTTATCAGATCAATTTTTGCGCGCACACTTGATGCAGCAAACCACTCAATTCC |
| | TGATTTGCAAAGAGCAGCTGTCACCATACTTGATGGTATTTCTGAACAGTCATTACGTCTTGTCGAC |
| | GCCATGGTTTATACTTCAGACCTGCTCACCAACAGTGTCATTATTATGGCATATGTAACTGGTGGTC |
| | TTGTACAACAGACTTCTCAGTGGTTGTCTAATCTTTTGGGCACTACTGTTGAAAAACTCAGGCCTAT |
| | CTTTGAATGGATTGAGGCGAAACTTAGTGCAGGAGTTGAATTTCTCAAGGATGCTTGGGAGATTCTC |
| | AAATTTCTCATTACAGGTGTTTTTGACATCGTCAAGGGTCAAATACAGGTTGCTTCAGATAACATCA |
| | AGGATTGTGTAAAATGCTTCATTGATGTTGTTAACAAGGCACTCGAAATGTGCATTGATCAAGTCAC |
| | TATCGCTGGCGCAAAGTTGCGATCACTCAACTTAGGTGAAGTCTTCATCGCTCAAAGCAAGGGACTT |
| | TACCGTCAGTGTATACGTGGCAAGGAGCAGCTGCAACTACTCATGCCTCTTAAGGCACCAAAAGAAG |
| | TAACCTTTCTTGAAGGTGATTCACATGACACAGTACTTACCTCTGAGGAGGTTGTTCTCAAGAACGG |
| | TGAACTCGAAGCACTCGAGACGCCCGTTGATAGCTTCACAAATGGAGCTATCGTTGGCACACCAGTC |
| | TGTGTAAATGGCCTCATGCTCTTAGAGATTAAGGACAAAGAACAATACTGCGCATTGTCTCCTGGTT |
| | TACTGGCTACAAACAATGTCTTTCGCTTAAAAGGGGGTGCACCAATTAAAGGTGTAACCTTTGGAGA |
| | AGATACTGTTTGGGAAGTTCAAGGTTACAAGAATGTGAGAATCACATTTGAGCTTGATGAACGTGTT |
| | GACAAAGTGCTTAATGAAAAGTGCTCTGTCTACACTGTTGAATCCGGTACCGAAGTTACTGAGTTTG |
| | CATGTGTTGTAGCAGAGGCTGTTGTGAAGACTTTACAACCAGTTTCTGATCTCCTTACCAACATGGG |
| | TATTGATCTTGATGAGTGGAGTGTAGCTACATTCTACTTATTTGATGATGCTGGTGAAGAAAACTTT |
| | TCATCACGTATGTATTGTTCCTTTTACCCTCCAGATGAGGAAGAAGAGGACGATGCAGAGTGTGAGG |
| | AAGAAGAAATTGATGAAACCTGTGAACATGAGTACGGTACAGAGGATGATTATCAAGGTCTCCCTCT |
| | GGAATTTGGTGCCTCAGCTGAAACAGTTCGAGTTGAGGAAGAAGAAGAGGAAGACTGGCTGGATGAT |
| | ACTACTGAGCAATCAGAGATTGAGCCAGAACCAGAACCTACACCTGAAGAACCAGTTAATCAGTTTA |
| | CTGGTTATTTAAAACTTACTGACAATGTTGCCATTAAATGTGTTGACATCGTTAAGGAGGCACAAAG |
| | TGCTAATCCTATGGTGATTGTAAATGCTGCTAACATACACCTGAAACATGGTGGTGGTGTAGCAGGT |
| | GCACTCAACAAGGCAACCAATGGTGCCATGCAAAAGGAGAGTGATGATTACATTAAGCTAAATGGCC |
| | CTCTTACAGTAGGAGGGTCTTGTTTGCTTTCTGGACATAATCTTGCTAAGAAGTGTCTGCATGTTGT |
| | TGGACCTAACCTAAATGCAGGTGAGGACATCCAGCTTCTTAAGGCAGCATATGAAAATTTCAATTCA |
| | CAGGACATCTTACTTGCACCATTGTTGTCAGCAGGCATATTTGGTGCTAAACCACTTCAGTCTTTAC |
| | AAGTGTGCGTGCAGACGGTTCGTACACAGGTTTATATTGCAGTCAATGACAAAGCTCTTTATGAGCA |
| | GGTTGTCATGGATTATCTTGATAACCTGAAGCCTAGAGTGGAAGCACCTAAACAAGAGGAGCCACCA |
| | AACACAGAAGATTCCAAAACTGAGGAGAAATCTGTCGTACAGAAGCCTGTCGATGTGAAGCCAAAAA |
| | TTAAGGCCTGCATTGATGAGGTTACCACAACACTGGAAGAAACTAAGTTTCTTACCAATAAGTTACT |
| | CTTGTTTGCTGATATCAATGGTAAGCTTTACCATGATTCTCAGAACATGCTTAGAGGTGAAGATATG |
| | TCTTTCCTTGAGAAGGATGCACCTTACATGGTAGGTGATGTTATCACTAGTGGTGATATCACTTGTG |
| | TTGTAATACCCTCCAAAAAGGCTGGTGGCACTACTGAGATGCTCTCAAGAGCTTTGAAGAAAGTGCC |
| | AGTTGATGAGTATATAACCACGTACCCTGGACAAGGATGTGCTGGTTATACACTTGAGGAAGCTAAG |
| | ACTGCTCTTAAGAAATGCAAATCTGCATTTTATGTACTACCTTCAGAAGCACCTAATGCTAAGGAAG |
| | AGATTCTAGGAACTGTATCCTGGAATTTGAGAGAAATGCTTGCTCATGCTGAAGAGACAAGAAAATT |
| | AATGCCTATATGCATGGATGTTAGAGCCATAATGGCAACCATCCAACGTAAGTATAAAGGAATTAAA |
| | ATTCAAGAGGGCATCGTTGACTATGGTGTCCGATTCTTCTTTTATACTAGTAAAGAGCCTGTAGCTT |
| | CTATTATTACGAAGCTGAACTCTCTAAATGAGCCGCTTGTCACAATGCCAATTGGTTATGTGACACA |
| | TGGTTTTAATCTTGAAGAGGCTGCGCGCTGTATGCGTTCTCTTAAAGCTCCTGCCGTAGTGTCAGTA |
| | TCATCACCAGATGCTGTTACTACATATAATGGATACCTCACTTCGTCATCAAAGACATCTGAGGAGC |
| | ACTTTGTAGAAACAGTTTCTTTGGCTGGCTCTTACAGAGATTGGTCCTATTCAGGACAGCGTACAGA |
| | GTTAGGTGTTGAATTTCTTAAGCGTGGTGACAAAATTGTGTACCACACTCTGGAGAGCCCCGTCGAG |
| | TTTCATCTTGACGGTGAGGTTCTTTCACTTGACAAACTAAAGAGTCTCTTATCCCTGCGGGAGGTTA |
| | AGACTATAAAAGTGTTCACAACTGTGGACAACACTAATCTCCACACACAGCTTGTGGATATGTCTAT |
| | GACATATGGACAGCAGTTTGGTCCAACATACTTGGATGGTGCTGATGTTACAAAAATTAAACCTCAT |
| | GTAAATCATGAGGGTAAGACTTTCTTTGTACTACCTAGTGATGACACACTACGTAGTGAAGCTTTCG |
| | AGTACTACCATACTCTTGATGAGAGTTTTCTTGGTAGGTACATGTCTGCTTTAAACCACACAAAGAA |
| | ATGGAAATTTCCTCAAGTTGGTGGTTTAACTTCAATTAAATGGGCTGATAACAATTGTTATTTGTCT |
| | AGTGTTTTATTAGCACTTCAACAGCTTGAAGTCAAATTCAATGCACCAGCACTTCAAGAGGCTTATT |
| | ATAGAGCCCGTGCTGGTGATGCTGCTAACTTTTGTGCACTCATACTCGCTTACAGTAATAAAACTGT |
| | TGGCGAGCTTGGTGATGTCAGAGAAACTATGACCCATCTTCTACAGCATGCTAATTTGGAATCTGCA |
| | AAGCGAGTTCTTAATGTGGTGTGTAAACATTGTGGTCAGAAAACTACTACCTTAACGGGTGTAGAAG |
| | CTGTGATGTATATGGGTACTCTATCTTATGATAATCTTAAGACAGGTGTTTCCATTCCATGTGTGTG |
| | TGGTCGTGATGCTACACAATATCTAGTACAACAAGAGTCTTCTTTTGTTATGATGTCTGCACCACCT |
| | GCTGAGTATAAATTACAGCAAGGTACATTCTTATGTGCGAATGAGTACACTGGTAACTATCAGTGTG |
| | GTCATTACACTCATATAACTGCTAAGGAGACCCTCTATCGTATTGACGGAGCTCACCTTACAAAGAT |
| | GTCAGAGTACAAAGGACCAGTGACTGATGTTTTCTACAAGGAAACATCTTACACTACAACCATCAAG |
| | CCTGTGTCGTATAAACTCGATGGAGTTACTTACACAGAGATTGAACCAAAATTGGATGGGTATTATA |
| | AAAAGGATAATGCTTACTATACAGAGCAGCCTATAGACCTTGTACCAACTCAACCATTACCAAATGC |
| | GAGTTTTGATAATTTCAAACTCACATGTTCTAACACAAAATTTGCTGATGATTTAAATCAAATGACA |
| | GGCTTCACAAAGCCAGCTTCACGAGAGCTATCTGTCACATTCTTCCCAGACTTGAATGGCGATGTAG |
| | TGGCTATTGACTATAGACACTATTCAGCGAGTTTCAAGAAAGGTGCTAAATTACTGCATAAGCCAAT |
| | TGTTTGGCACATTAACCAGGCTACAACCAAGACAACGTTCAAACCAAACACTTGGTGTTTACGTTGT |
| | CTTTGGAGTACAAAGCCAGTAGATACTTCAAATTCATTTGAAGTTCTGGCAGTAGAAGACACACAAG |
| | GAATGGACAATCTTGCTTGTGAAAGTCAACAACCCACCTCTGAAGAAGTAGTGGAAAATCCTACCAT |
| | ACAGAAGGAAGTCATAGAGTGTGACGTGAAAACTACCGAAGTTGTAGGCAATGTCATACTTAAACCA |
| | TCAGATGAAGGTGTTAAAGTAACACAAGAGTTAGGTCATGAGGATCTTATGGCTGCTTATGTGGAAA |
| | ACACAAGCATTACCATTAAGAAACCTAATGAGCTTTCACTAGCCTTAGGTTTAAAAACAATTGCCAC |
| | TCATGGTATTGCTGCAATTAATAGTGTTCCTTGGAGTAAAATTTTGGCTTATGTCAAACCATTCTTA |
| | GGACAAGCAGCAATTACAACATCAAATTGCGCTAAGAGATTAGCACAACGTGTGTTTAACAATTATA |
| | TGCCTTATGTGTTTACATTATTGTTCCAATTGTGTACTTTTACTAAAAGTACCAATTCTAGAATTAG |
| | AGCTTCACTACCTACAACTATTGCTAAAAATAGTGTTAAGAGTGTTGCTAAATTATGTTTGGATGCC |
| | GGCATTAATTATGTGAAGTCACCCAAATTTTCTAAATTGTTCACAATCGCTATGTGGCTATTGTTGT |
| | TAAGTATTTGCTTAGGTTCTCTAATCTGTGTAACTGCTGCTTTTGGTGTACTCTTATCTAATTTTGG |
| | TGCTCCTTCTTATTGTAATGGCGTTAGAGAATTGTATCTTAATTCGTCTAACGTTACTACTATGGAT |
| | TTCTGTGAAGGTTCTTTTCCTTGCAGCATTTGTTTAAGTGGATTAGACTCCCTTGATTCTTATCCAG |
| | CTCTTGAAACCATTCAGGTGACGATTTCATCGTACAAGCTAGACTTGACAATTTTAGGTCTGGCCGC |
| | TGAGTGGGTTTTGGCATATATGTTGTTCACAAAATTCTTTTATTTATTAGGTCTTTCAGCTATAATG |
| | CAGGTGTTCTTTGGCTATTTTGCTAGTCATTTCATCAGCAATTCTTGGCTCATGTGGTTTATCATTA |
| | GTATTGTACAAATGGCACCCGTTTCTGCAATGGTTAGGATGTACATCTTCTTTGCTTCTTTCTACTA |
| | CATATGGAAGAGCTATGTTCATATCATGGATGGTTGCACCTCTTCGACTTGCATGATGTGCTATAAG |
| | CGCAATCGTGCCACACGCGTTGAGTGTACAACTATTGTTAATGGCATGAAGAGATCTTTCTATGTCT |
| | ATGCAAATGGAGGCCGTGGCTTCTGCAAGACTCACAATTGGAATTGTCTCAATTGTGACACATTTTG |
| | CACTGGTAGTACATTCATTAGTGATGAAGTTGCTCGTGATTTGTCACTCCAGTTTAAAAGACCAATC |
| | AACCCTACTGACCAGTCATCGTATATTGTTGATAGTGTTGCTGTGAAAAATGGCGCGCTTCACCTCT |
| | ACTTTGACAAGGCTGGTCAAAAGACCTATGAGAGACATCCGCTCTCCCATTTTGTCAATTTAGACAA |
| | TTTGAGAGCTAACAACACTAAAGGTTCACTGCCTATTAATGTCATAGTTTTTGATGGCAAGTCCAAA |
| | TGCGACGAGTCTGCTTCTAAGTCTGCTTCTGTGTACTACAGTCAGCTGATGTGCCAACCTATTCTGT |
| | TGCTTGACCAAGTTCTTGTATCAGACGTTGGAGATAGTACTGAAGTTTCCGTTAAGATGTTTGATGC |
| | TTATGTCGACACCTTTTCAGCAACTTTTAGTGTTCCTATGGAAAAACTTAAGGCACTTGTTGCTACA |
| | GCTCACAGCGAGTTAGCAAAGGGTGTAGCTTTAGATGGTGTCCTTTCTACATTCGTGTCAGCTGCCC |
| | GACAAGGTGTTGTTGATACCGATGTTGACACAAAGGATGTTATTGAATGTCTCAAACTTTCACATCA |
| | CTCTGACTTAGAAGTGACAGGTGACAGTTGTAACAATTTCATGCTCACCTATAATAAGGTTGAAAAC |
| | ATGACGCCCAGAGATCTTGGCGCATGTATTGACTGTAATGCAAGGCATATCAATGCCCAAGTAGCAA |
| | AAAGTCACAATGTTTCACTCATCTGGAATGTAAAAGACTACATGTCTTTATCTGAACAGCTGCGTAA |
| | ACAAATTCGTAGTGCTGCCAAGAAGAACAACATACCTTTTAGACTAACTTGTGCTACAACTAGACAG |
| | GTTGTCAATGTCATAACTACTAAAATCTCACTCAAGGGTGGTAAGATTGTTAGTACTTGTTTTAAAC |
| | TTATGCTTAAGGCCACATTATTGTGCGTTCTTGCTGCATTGGTTTGTTATATCGTTATGCCAGTACA |
| | TACATTGTCAATCCATGATGGTTACACAAATGAAATCATTGGTTACAAAGCCATTCAGGATGGTGTC |
| | ACTCGTGACATCATTTCTACTGATGATTGTTTTGCAAATAAACATGCTGGTTTTGACGCATGGTTTA |
| | GCCAGCGTGGTGGTTCATACAAAAATGACAAAAGCTGCCCTGTAGTAGCTGCTATCATTACAAGAGA |
| | GATTGGTTTCATAGTGCCTGGCTTACCGGGTACTGTGCTGAGAGCAATCAATGGTGACTTCTTGCAT |
| | TTTCTACCTCGTGTTTTTAGTGCTGTTGGCAACATTTGCTACACACCTTCCAAACTCATTGAGTATA |
| | GTGATTTTGCTACCTCTGCTTGCGTTCTTGCTGCTGAGTGTACAATTTTTAAGGATGCTATGGGCAA |
| | ACCTGTGCCATATTGTTATGACACTAATTTGCTAGAGGGTTCTATTTCTTATAGTGAGCTTCGTCCA |
| | GACACTCGTTATGTGCTTATGGATGGTTCCATCATACAGTTTCCTAACACTTACCTGGAGGGTTCTG |
| | TTAGAGTAGTAACAACTTTTGATGCTGAGTACTGTAGACATGGTACATGCGAAAGGTCAGAAGTAGG |
| | TATTTGCCTATCTACCAGTGGTAGATGGGTTCTTAATAATGAGCATTACAGAGCTCTATCAGGAGTT |
| | TTCTGTGGTGTTGATGCGATGAATCTCATAGCTAACATCTTTACTCCTCTTGTGCAACCTGTGGGTG |
| | CTTTAGATGTGTCTGCTTCAGTAGTGGCTGGTGGTATTATTGCCATATTGGTGACTTGTGCTGCCTA |
| | CTACTTTATGAAATTCAGACGTGTTTTTGGTGAGTACAACCATGTTGTTGCTGCTAATGCACTTTTG |
| | TTTTTGATGTCTTTCACTATACTCTGTCTGGTACCAGCTTACAGCTTTCTGCCGGGAGTCTACTCAG |
| | TCTTTTACTTGTACTTGACATTCTATTTCACCAATGATGTTTCATTCTTGGCTCACCTTCAATGGTT |
| | TGCCATGTTTTCTCCTATTGTGCCTTTTTGGATAACAGCAATCTATGTATTCTGTATTTCTCTGAAG |
| | CACTGCCATTGGTTCTTTAACAACTATCTTAGGAAAAGAGTCATGTTTAATGGAGTTACATTTAGTA |
| | CCTTCGAGGAGGCTGCTTTGTGTACCTTTTTGCTCAACAAGGAAATGTACCTAAAATTGCGTAGCGA |
| | GACACTGTTGCCACTTACACAGTATAACAGGTATCTTGCTCTATATAACAAGTACAAGTATTTCAGT |
| | GGAGCCTTAGATACTACCAGCTATCGTGAAGCAGCTTGCTGCCACTTAGCAAAGGCTCTAAATGACT |
| | TTAGCAACTCAGGTGCTGATGTTCTCTACCAACCACCACAGACATCAATCACTTCTGCTGTTCTGCA |
| | GAGTGGTTTTAGGAAAATGGCATTCCCGTCAGGCAAAGTTGAAGGGTGCATGGTACAAGTAACCTGT |
| | GGAACTACAACTCTTAATGGATTGTGGTTGGATGACACAGTATACTGTCCAAGACATGTCATTTGCA |
| | CAGCAGAAGACATGCTTAATCCTAACTATGAAGATCTGCTCATTCGCAAATCCAACCATAGCTTTCT |
| | TGTTCAGGCTGGCAATGTTCAACTTCGTGTTATTGGCCATTCTATGCAAAATTGTCTGCTTAGGCTT |
| | AAAGTTGATACTTCTAACCCTAAGACACCCAAGTATAAATTTGTCCGTATCCAACCTGGTCAAACAT |
| | TTTCAGTTCTAGCATGCTACAATGGTTCACCATCTGGTGTTTATCAGTGTGCCATGAGACCTAATCA |
| | TACCATTAAAGGTTCTTTCCTTAATGGATCATGTGGTAGTGTTGGTTTTAACATTGATTATGATTGC |
| | GTGTCTTTCTGCTATATGCATCATATGGAGCTTCCAACAGGAGTACACGCTGGTACTGACTTAGAAG |
| | GTAAATTCTATGGTCCATTTGTTGACAGACAAACTGCACAGGCTGCAGGTACAGACACAACCATAAC |
| | ATTAAATGTTTTGGCATGGCTGTATGCTGCTGTTATCAATGGTGATAGGTGGTTTCTTAATAGATTC |
| | ACCACTACTTTGAATGACTTTAACCTTGTGGCAATGAAGTACAACTATGAACCTTTGACACAAGATC |
| | ATGTTGACATATTGGGACCTCTTTCTGCTCAAACAGGAATTGCCGTCTTAGATATGTGTGCTGCTTT |
| | GAAAGAGCTGCTGCAGAATGGTATGAATGGTCGTACTATCCTTGGTAGCACTATTTTAGAAGATGAG |
| | TTTACACCATTTGATGTTGTTAGACAATGCTCTGGTGTTACCTTCCAAGGTAAGTTCAAGAAAATTG |
| | TTAAGGGCACTCATCATTGGATGCTTTTAACTTTCTTGACATCACTATTGATTCTTGTTCAAAGTAC |
| | ACAGTGGTCACTGTTTTTCTTTGTTTACGAGAATGCTTTCTTGCCATTTACTCTTGGTATTATGGCA |
| | ATTGCTGCATGTGCTATGCTGCTTGTTAAGCATAAGCACGCATTCTTGTGCTTGTTTCTGTTACCTT |
| | CTCTTGCAACAGTTGCTTACTTTAATATGGTCTACATGCCTGCTAGCTGGGTGATGCGTATCATGAC |
| | ATGGCTTGAATTGGCTGACACTAGCTTGTCTGGTTATAGGCTTAAGGATTGTGTTATGTATGCTTCA |
| | GCTTTAGTTTTGCTTATTCTCATGACAGCTCGCACTGTTTATGATGATGCTGCTAGACGTGTTTGGA |
| | CACTGATGAATGTCATTACACTTGTTTACAAAGTCTACTATGGTAATGCTTTAGATCAAGCTATTTC |
| | CATGTGGGCCTTAGTTATTTCTGTAACCTCTAACTATTCTGGTGTCGTTACGACTATCATGTTTTTA |
| | GCTAGAGCTATAGTGTTTGTGTGTGTTGAGTATTACCCATTGTTATTTATTACTGGCAACACCTTAC |
| | AGTGTATCATGCTTGTTTATTGTTTCTTAGGCTATTGTTGCTGCTGCTACTTTGGCCTTTTCTGTTT |
| | ACTCAACCGTTACTTCAGGCTTACTCTTGGTGTTTATGACTACTTGGTCTCTACACAAGAATTTAGG |
| | TATATGAACTCCCAGGGGCTTTTGCCTCCTAAGAGTAGTATTGATGCTTTCAAGCTTAACATTAAGT |
| | TGTTGGGTATTGGAGGTAAACCATGTATCAAGGTTGCTACTGTACAGTCTAAAATGTCTGACGTAAA |
| | GTGCACATCTGTGGTACTGCTCTCGGTTCTTCAACAACTTAGAGTAGAGTCATCTTCTAAATTGTGG |
| | GCACAATGTGTACAACTCCACAATGATATTCTTCTTGCAAAAGACACAACTGAAGCTTTCGAGAAGA |
| | TGGTTTCTCTTTTGTCTGTTTTGCTATCCATGCAGGGTGCTGTAGACATTAATAGGTTGTGCGAGGA |
| | AATGCTCGATAACCGTGCTACTCTTCAGGCTATTGCTTCAGAATTTAGTTCTTTACCATCATATGCC |
| | GCTTATGCCACTGCCCAGGAGGCCTATGAGCAGGCTGTAGCTAATGGTGATTCTGAAGTCGTTCTCA |
| | AAAAGTTAAAGAAATCTTTGAATGTGGCTAAATCTGAGTTTGACCGTGATGCTGCCATGCAACGCAA |
| | GTTGGAAAAGATGGCAGATCAGGCTATGACCCAAATGTACAAACAGGCAAGATCTGAGGACAAGAGG |
| | GCAAAAGTAACTAGTGCTATGCAAACAATGCTCTTCACTATGCTTAGGAAGCTTGATAATGATGCAC |
| | TTAACAACATTATCAACAATGCGCGTGATGGTTGTGTTCCACTCAACATCATACCATTGACTACAGC |
| | AGCCAAACTCATGGTTGTTGTCCCTGATTATGGTACCTACAAGAACACTTGTGATGGTAACACCTTT |
| | ACATATGCATCTGCACTCTGGGAAATCCAGCAAGTTGTTGATGCGGATAGCAAGATTGTTCAACTTA |
| | GTGAAATTAACATGGACAATTCACCAAATTTGGCTTGGCCTCTTATTGTTACAGCTCTAAGAGCCAA |
| | CTCAGCTGTTAAACTACAGAATAATGAACTGAGTCCAGTAGCACTACGACAGATGTCCTGTGCGGCT |
| | GGTACCACACAAACAGCTTGTACTGATGACAATGCACTTGCCTACTATAACAATTCGAAGGGAGGTA |
| | GGTTTGTGCTGGCATTACTATCAGACCACCAAGATCTCAAATGGGCTAGATTCCCTAAGAGTGATGG |
| | TACAGGTACAATTTACACAGAACTGGAACCACCTTGTAGGTTTGTTACAGACACACCAAAAGGGCCT |
| | AAAGTGAAATACTTGTACTTCATCAAAGGCTTAAACAACCTAAATAGAGGTATGGTGCTGGGCAGTT |
| | TAGCTGCTACAGTACGTCTTCAGGCTGGAAATGCTACAGAAGTACCTGCCAATTCAACTGTGCTTTC |
| | CTTCTGTGCTTTTGCAGTAGACCCTGCTAAAGCATATAAGGATTACCTAGCAAGTGGAGGACAACCA |
| | ATCACCAACTGTGTGAAGATGTTGTGTACACACACTGGTACAGGACAGGCAATTACTGTAACACCAG |
| | AAGCTAACATGGACCAAGAGTCCTTTGGTGGTGCTTCATGTTGTCTGTATTGTAGATGCCACATTGA |
| | CCATCCAAATCCTAAAGGATTCTGTGACTTGAAAGGTAAGTACGTCCAAATACCTACCACTTGTGCT |
| | AATGACCCAGTGGGTTTTACACTTAGAAACACAGTCTGTACCGTCTGCGGAATGTGGAAAGGTTATG |
| | GCTGTAGTTGTGACCAACTCCGCGAACCCTTGATGCAGTCTGCGGATGCATCAACGTTTTTAAACGG |
| | GTTTGCGGTGTAAGTGCAGCCCGTCTTACACCGTGCGGCACAGGCACTAGTACTGATGTCGTCTACA |
| | GGGCTTTTGATATTTACAACGAAAAAGTTGCTGGTTTTGCAAAGTTCCTAAAAACTAATTGCTGTCG |
| | CTTCCAGGAGAAGGATGAGGAAGGCAATTTATTAGACTCTTACTTTGTAGTTAAGAGGCATACTATG |
| | TCTAACTACCAACATGAAGAGACTATTTATAACTTGGTTAAAGATTGTCCAGCGGTTGCTGTCCATG |
| | ACTTTTTCAAGTTTAGAGTAGATGGTGACATGGTACCACATATATCACGTCAGCGTCTAACTAAATA |
| | CACAATGGCTGATTTAGTCTATGCTCTACGTCATTTTGATGAGGGTAATTGTGATACATTAAAAGAA |
| | ATACTCGTCACATACAATTGCTGTGATGATGATTATTTCAATAAGAAGGATTGGTATGACTTCGTAG |
| | AGAATCCTGACATCTTACGCGTATATGCTAACTTAGGTGAGCGTGTACGCCAATCATTATTAAAGAC |
| | TGTACAATTCTGCGATGCTATGCGTGATGCAGGCATTGTAGGCGTACTGACATTAGATAATCAGGAT |
| | CTTAATGGGAACTGGTACGATTTCGGTGATTTCGTACAAGTAGCACCAGGCTGCGGAGTTCCTATTG |
| | TGGATTCATATTACTCATTGCTGATGCCCATCCTCACTTTGACTAGGGCATTGGCTGCTGAGTCCCA |
| | TATGGATGCTGATCTCGCAAAACCACTTATTAAGTGGGATTTGCTGAAATATGATTTTACGGAAGAG |
| | AGACTTTGTCTCTTCGACCGTTATTTTAAATATTGGGACCAGACATACCATCCCAATTGTATTAACT |
| | GTTTGGATGATAGGTGTATCCTTCATTGTGCAAACTTTAATGTGTTATTTTCTACTGTGTTTCCACC |
| | TACAAGTTTTGGACCACTAGTAAGAAAAATATTTGTAGATGGTGTTCCTTTTGTTGTTTCAACTGGA |
| | TACCATTTTCGTGAGTTAGGAGTCGTACATAATCAGGATGTAAACTTACATAGCTCGCGTCTCAGTT |
| | TCAAGGAACTTTTAGTGTATGCTGCTGATCCAGCTATGCATGCAGCTTCTGGCAATTTATTGCTAGA |
| | TAAACGCACTACATGCTTTTCAGTAGCTGCACTAACAAACAATGTTGCTTTTCAAACTGTCAAACCC |
| | GGTAATTTTAATAAAGACTTTTATGACTTTGCTGTGTCTAAAGGTTTCTTTAAGGAAGGAAGTTCTG |
| | TTGAACTAAAACACTTCTTCTTTGCTCAGGATGGCAACGCTGCTATCAGTGATTATGACTATTATCG |
| | TTATAATCTGCCAACAATGTGTGATATCAGACAACTCCTATTCGTAGTTGAAGTTGTTGATAAATAC |
| | TTTGATTGTTACGATGGTGGCTGTATTAATGCCAACCAAGTAATCGTTAACAATCTGGATAAATCAG |
| | CTGGTTTCCCATTTAATAAATGGGGTAAGGCTAGACTTTATTATGACTCAATGAGTTATGAGGATCA |
| | AGATGCACTTTTCGCGTATACTAAGCGTAATGTCATCCCTACTATAACTCAAATGAATCTTAAGTAT |
| | GCCATTAGTGCAAAGAATAGAGCTCGCACCGTAGCTGGTGTCTCTATCTGTAGTACTATGACAAATA |
| | GACAGTTTCATCAGAAATTATTGAAGTCAATAGCCGCCACTAGAGGAGCTACTGTGGTAATTGGAAC |
| | AAGCAAGTTTTACGGTGGCTGGCATAATATGTTAAAAACTGTTTACAGTGATGTAGAAACTCCACAC |
| | CTTATGGGTTGGGATTATCCAAAATGTGACAGAGCCATGCCTAACATGCTTAGGATAATGGCCTCTC |
| | TTGTTCTTGCTCGCAAACATAACACTTGCTGTAACTTATCACACCGTTTCTACAGGTTAGCTAACGA |
| | GTGTGCGCAAGTATTAAGTGAGATGGTCATGTGTGGCGGCTCACTATATGTTAAACCAGGTGGAACA |
| | TCATCCGGTGATGCTACAACTGCTTATGCTAATAGTGTCTTTAACATTTGTCAAGCTGTTACAGCCA |
| | ATGTAAATGCACTTCTTTCAACTGATGGTAATAAGATAGCTGACAAGTATGTCCGCAATCTACAACA |
| | CAGGCTCTATGAGTGTCTCTATAGAAATAGGGATGTTGATCATGAATTCGTGGATGAGTTTTACGCT |
| | TACCTGCGTAAACATTTCTCCATGATGATTCTTTCTGATGATGCCGTTGTGTGCTATAACAGTAACT |
| | ATGCGGCTCAAGGTTTAGTAGCTAGCATTAAGAACTTTAAGGCAGTTCTTTATTATCAAAATAATGT |
| | GTTCATGTCTGAGGCAAAATGTTGGACTGAGACTGACCTTACTAAAGGACCTCACGAATTTTGCTCA |
| | CAGCATACAATGCTAGTTAAACAAGGAGATGATTACGTGTACCTGCCTTACCCAGATCCATCAAGAA |
| | TATTAGGCGCAGGCTGTTTTGTCGATGATATTGTCAAAACAGATGGTACACTTATGATTGAAAGGTT |
| | CGTGTCACTGGCTATTGATGCTTACCCACTTACAAAACATCCTAATCAGGAGTATGCTGATGTCTTT |
| | CACTTGTATTTACAATACATTAGAAAGTTACATGATGAGCTTACTGGCCACATGTTGGACATGTATT |
| | CCGTAATGCTAACTAATGATAACACCTCACGGTACTGGGAACCTGAGTTTTATGAGGCTATGTACAC |
| | ACCACATACAGTCTTGCAGGCTGTAGGTGCTTGTGTATTGTGCAATTCACAGACTTCACTTCGTTGC |
| | GGTGCCTGTATTAGGAGACCATTCCTATGTTGCAAGTGCTGCTATGACCATGTCATTTCAACATCAC |
| | ACAAATTAGTGTTGTCTGTTAATCCCTATGTTTGCAATGCCCCAGGTTGTGATGTCACTGATGTGAC |
| | ACAACTGTATCTAGGAGGTATGAGCTATTATTGCAAGTCACATAAGCCTCCCATTAGTTTTCCATTA |
| | TGTGCTAATGGTCAGGTTTTTGGTTTATACAAAAACACATGTGTAGGCAGTGACAATGTCACTGACT |
| | TCAATGCGATAGCAACATGTGATTGGACTAATGCTGGCGATTACATACTTGCCAACACTTGTACTGA |
| | GAGACTCAAGCTTTTCGCAGCAGAAACGCTCAAAGCCACTGAGGAAACATTTAAGCTGTCATATGGT |
| | ATTGCTACTGTACGCGAAGTACTCTCTGACAGAGAATTGCATCTTTCATGGGAGGTTGGAAAACCTA |
| | GACCACCATTGAACAGAAACTATGTCTTTACTGGTTACCGTGTAACTAAAAATAGTAAAGTACAGAT |
| | TGGAGAGTACACCTTTGAAAAAGGTGACTATGGTGATGCTGTTGTGTACAGAGGTACTACGACATAC |
| | AAGTTGAATGTTGGTGATTACTTTGTGTTGACATCTCACACTGTAATGCCACTTAGTGCACCTACTC |
| | TAGTGCCACAAGAGCACTATGTGAGAATTACTGGCTTGTACCCAACACTCAACATCTCAGATGAGTT |
| | TTCTAGCAATGTTGCAAATTATCAAAAGGTCGGCATGCAAAAGTACTCTACACTCCAAGGACCACCT |
| | GGTACTGGTAAGAGTCATTTTGCCATCGGACTTGCTCTCTATTACCCATCTGCTCGCATAGTGTATA |
| | CGGCATGCTCTCATGCAGCTGTTGATGCCCTATGTGAAAAGGCATTAAAATATTTGCCCATAGATAA |
| | ATGTAGTAGAATCATACCTGCGCGTGCGCGCGTAGAGTGTTTTGATAAATTCAAAGTGAATTCAACA |
| | CTAGAACAGTATGTTTTCTGCACTGTAAATGCATTGCCAGAAACAACTGCTGACATTGTAGTCTTTG |
| | ATGAAATCTCTATGGCTACTAATTATGACTTGAGTGTTGTCAATGCTAGACTTCGTGCAAAACACTA |
| | CGTCTATATTGGCGATCCTGCTCAATTACCAGCCCCCCGCACATTGCTGACTAAAGGCACACTAGAA |
| | CCAGAATATTTTAATTCAGTGTGCAGACTTATGAAAACAATAGGTCCAGACATGTTCCTTGGAACTT |
| | GTCGCCGTTGTCCTGCTGAAATTGTTGACACTGTGAGTGCTTTAGTTTATGACAATAAGCTAAAAGC |
| | ACACAAGGATAAGTCAGCTCAATGCTTCAAAATGTTCTACAAAGGTGTTATTACACATGATGTTTCA |
| | TCTGCAATCAACAGACCTCAAATAGGCGTTGTAAGAGAATTTCTTACACGCAATCCTGCTTGGAGAA |
| | AAGCTGTTTTTATCTCACCTTATAATTCACAGAACGCTGTAGCTTCAAAAATCTTAGGATTGCCTAC |
| | GCAGACTGTTGATTCATCACAGGGTTCTGAATATGACTATGTCATATTCACACAAACTACTGAAACA |
| | GCACACTCTTGTAATGTCAACCGCTTCAATGTGGCTATCACAAGGGCAAAAATTGGCATTTTGTGCA |
| | TAATGTCTGATAGAGATCTTTATGACAAACTGCAATTTACAAGTCTAGAAATACCACGTCGCAATGT |
| | GGCTACATTACAAGCAGAAAATGTAACTGGACTTTTTAAGGACTGTAGTAAGATCATTACTGGTCTT |
| | CATCCTACACAGGCACCTACACACCTCAGCGTTGATATAAAGTTCAAGACTGAAGGATTATGTGTTG |
| | ACATACCAGGCATACCAAAGGACATGACCTACCGTAGACTCATCTCTATGATGGGTTTCAAAATGAA |
| | TTACCAAGTCAATGGTTACCCTAATATGTTTATCACCCGCGAAGAAGCTATTCGTCACGTTCGTGCG |
| | TGGATTGGCTTTGATGTAGAGGGCTGTCATGCAACTAGAGATGCTGTGGGTACTAACCTACCTCTCC |
| | AGCTAGGATTTTCTACAGGTGTTAACTTAGTAGCTGTACCGACTGGTTATGTTGACACTGAAAATAA |
| | CACAGAATTCACCAGAGTTAATGCAAAACCTCCACCAGGTGACCAGTTTAAACATCTTATACCACTC |
| | ATGTATAAAGGCTTGCCCTGGAATGTAGTGCGTATTAAGATAGTACAAATGCTCAGTGATACACTGA |
| | AAGGATTGTCAGACAGAGTCGTGTTCGTCCTTTGGGCGCATGGCTTTGAGCTTACATCAATGAAGTA |
| | CTTTGTCAAGATTGGACCTGAAAGAACGTGTTGTCTGTGTGACAAACGTGCAACTTGCTTTTCTACT |
| | TCATCAGATACTTATGCCTGCTGGAATCATTCTGTGGGTTTTGACTATGTCTATAACCCATTTATGA |
| | TTGATGTTCAGCAGTGGGGCTTTACGGGTAACCTTCAGAGTAACCATGACCAACATTGCCAGGTACA |
| | TGGAAATGCACATGTGGCTAGTTGTGATGCTATCATGACTAGATGTTTAGCAGTCCATGAGTGCTTT |
| | GTTAAGCGCGTTGATTGGTCTGTTGAATACCCTATTATAGGAGATGAACTGAGGGTTAATTCTGCTT |
| | GCAGAAAAGTACAACACATGGTTGTGAAGTCTGCATTGCTTGCTGATAAGTTTCCAGTTCTTCATGA |
| | CATTGGAAATCCAAAGGCTATCAAGTGTGTGCCTCAGGCTGAAGTAGAATGGAAGTTCTACGATGCT |
| | CAGCCATGTAGTGACAAAGCTTACAAAATAGAGGAGCTCTTCTATTCTTATGCTACACATCACGATA |
| | AATTCACTGATGGTGTTTGTTTGTTTTGGAATTGTAACGTTGATCGTTACCCAGCCAATGCAATTGT |
| | GTGTAGGTTTGACACAAGAGTCTTGTCAAACTTGAACTTACCAGGCTGTGATGGTGGTAGTTTGTAT |
| | GTGAATAAGCATGCATTCCACACTCCAGCTTTCGATAAAAGTGCATTTACTAATTTAAAGCAATTGC |
| | CTTTCTTTTACTATTCTGATAGTCCTTGTGAGTCTCATGGCAAACAAGTAGTGTCGGATATTGATTA |
| | TGTTCCACTCAAATCTGCTACGTGTATTACACGATGCAATTTAGGTGGTGCTGTTTGCAGACACCAT |
| | GCAAATGAGTACCGACAGTACTTGGATGCATATAATATGATGATTTCTGCTGGATTTAGCCTATGGA |
| | TTTACAAACAATTTGATACTTATAACCTGTGGAATACATTTACCAGGTTACAGAGTTTAGAAAATGT |
| | GGCTTATAATGTTGTTAATAAAGGACACTTTGATGGACACGCCGGCGAAGCACCTGTTTCCATCATT |
| | AATAATGCTGTTTACACAAAGGTAGATGGTATTGATGTGGAGATCTTTGAAAATAAGACAACACTTC |
| | CTGTTAATGTTGCATTTGAGCTTTGGGCTAAGCGTAACATTAAACCAGTGCCAGAGATTAAGATACT |
| | CAATAATTTGGGTGTTGATATCGCTGCTAATACTGTAATCTGGGACTACAAAAGAGAAGCCCCAGCA |
| | CATGTATCTACAATAGGTGTCTGCACAATGACTGACATTGCCAAGAAACCTACTGAGAGTGCTTGTT |
| | CTTCACTTACTGTCTTGTTTGATGGTAGAGTGGAAGGACAGGTAGACCTTTTTAGAAACGCCCGTAA |
| | TGGTGTTTTAATAACAGAAGGTTCAGTCAAAGGTCTAACACCTTCAAAGGGACCAGCACAAGCTAGC |
| | GTCAATGGAGTCACATTAATTGGAGAATCAGTAAAAACACAGTTTAACTACTTTAAGAAAGTAGACG |
| | GCATTATTCAACAGTTGCCTGAAACCTACTTTACTCAGAGCAGAGACTTAGAGGATTTTAAGCCCAG |
| | ATCACAAATGGAAACTGACTTTCTCGAGCTCGCTATGGATGAATTCATACAGCGATATAAGCTCGAG |
| | GGCTATGCCTTCGAACACATCGTTTATGGAGATTTCAGTCATGGACAACTTGGCGGTCTTCATTTAA |
| | TGATAGGCTTAGCCAAGCGCTCACAAGATTCACCACTTAAATTAGAGGATTTTATCCCTATGGACAG |
| | CACAGTGAAAAATTACTTCATAACAGATGCGCAAACAGGTTCATCAAAATGTGTGTGTTCTGTGATT |
| | GATCTTTTACTTGATGACTTTGTCGAGATAATAAAGTCACAAGATTTGTCAGTGATTTCAAAAGTGG |
| | TCAAGGTTACAATTGACTATGCTGAAATTTCATTCATGCTTTGGTGTAAGGATGGACATGTTGAAAC |
| | CTTCTACCCAAAACTACAAGCAAGTCAAGCGTGGCAACCAGGTGTTGCGATGCCTAACTTGTACAAG |
| | ATGCAAAGAATGCTTCTTGAAAAGTGTGACCTTCAGAATTATGGTGAAAATGCTGTTATACCAAAAG |
| | GAATAATGATGAATGTCGCAAAGTATACTCAACTGTGTCAATACTTAAATACACTTACTTTAGCTGT |
| | ACCCTACAACATGAGAGTTATTCACTTTGGTGCTGGCTCTGATAAAGGAGTTGCACCAGGTACAGCT |
| | GTGCTCAGACAATGGTTGCCAACTGGCACACTACTTGTCGATTCAGATCTTAATGACTTCGTCTCCG |
| | ACGCAGATTCTACTTTAATTGGAGACTGTGCAACAGTACATACGGCTAATAAATGGGACCTTATTAT |
| | TAGCGATATGTATGACCCTAGGACCAAACATGTGACAAAAGAGAATGACTCTAAAGAAGGGTTTTTC |
| | ACTTATCTGTGTGGATTTATAAAGCAAAAACTAGCCCTGGGTGGTTCTATAGCTGTAAAGATAACAG |
| | AGCATTCTTGGAATGCTGACCTTTACAAGCTTATGGGCCATTTCTCATGGTGGACAGCTTTTGTTAC |
| | AAATGTAAATGCATCATCATCGGAAGCATTTTTAATTGGGGCTAACTATCTTGGCAAGCCGAAGGAA |
| | CAAATTGATGGCTATACCATGCATGCTAACTACATTTTCTGGAGGAACACAAATCCTATCCAGTTGT |
| | CTTCCTATTCACTCTTTGACATGAGCAAATTTCCTCTTAAATTAAGAGGAACTGCTGTAATGTCTCT |
| | TAAGGAGAATCAAATCAATGATATGATTTATTCTCTTCTGGAAAAAGGTAGGCTTATCATTAGAGAA |
| | AACAACAGAGTTGTGGTTTCAAGTGATATTCTTGTTAACAACTAAACGAACATGTTTATTTTCTTAT |
| | TATTTCTTACTCTCACTAGTGGTAGTGACCTTGACCGGTGCACCACTTTTGATGATGTTCAAGCTCC |
| | TAATTACACTCAACATACTTCATCTATGAGGGGGGTTTACTATCCTGATGAAATTTTTAGATCAGAC |
| | ACTCTTTATTTAACTCAGGATTTATTTCTTCCATTTTATTCTAATGTTACAGGGTTTCATACTATTA |
| | ATCATACGTTTGGCAACCCTGTCATACCTTTTAAGGATGGTATTTATTTTGCTGCCACAGAGAAATC |
| | AAATGTTGTCCGTGGTTGGGTTTTTGGTTCTACCATGAACAACAAGTCACAGTCGGTGATTATTATT |
| | AACAATTCTACTAATGTTGTTATACGAGCATGTAACTTTGAATTGTGTGACAACCCTTTCTTTGCTG |
| | TTTCTAAACCCATGGGTACACAGACACATACTATGATATTCGATAATGCATTTAATTGCACTTTCGA |
| | GTACATATCTGATGCCTTTTCGCTTGATGTTTCAGAAAAGTCAGGTAATTTTAAACACTTACGAGAG |
| | TTTGTGTTTAAAAATAAAGATGGGTTTCTCTATGTTTATAAGGGCTATCAACCTATAGATGTAGTTC |
| | GTGATCTACCTTCTGGTTTTAACACTTTGAAACCTATTTTTAAGTTGCCTCTTGGTATTAACATTAC |
| | AAATTTTAGAGCCATTCTTACAGCCTTTTCACCTGCTCAAGACATTTGGGGCACGTCAGCTGCAGCC |
| | TATTTTGTTGGCTATTTAAAGCCAACTACATTTATGCTCAAGTATGATGAAAATGGTACAATCACAG |
| | ATGCTGTTGATTGTTCTCAAAATCCACTTGCTGAACTCAAATGCTCTGTTAAGAGCTTTGAGATTGA |
| | CAAAGGAATTTACCAGACCTCTAATTTCAGGGTTGTTCCCTCAGGAGATGTTGTGAGATTCCCTAAT |
| | ATTACAAACTTGTGTCCTTTTGGAGAGGTTTTTAATGCTACTAAATTCCCTTCTGTCTATGCATGGG |
| | AGAGAAAAAAAATTTCTAATTGTGTTGCTGATTACTCTGTGCTCTACAACTCAACATTTTTTTCAAC |
| | CTTTAAGTGCTATGGCGTTTCTGCCACTAAGTTGAATGATCTTTGCTTCTCCAATGTCTATGCAGAT |
| | TCTTTTGTAGTCAAGGGAGATGATGTAAGACAAATAGCGCCAGGACAAACTGGTGTTATTGCTGATT |
| | ATAATTATAAATTGCCAGATGATTTCATGGGTTGTGTCCTTGCTTGGAATACTAGGAACATTGATGC |
| | TACTTCAACTGGTAATTATAATTATAAATATAGGTATCTTAGACATGGCAAGCTTAGGCCCTTTGAG |
| | AGAGACATATCTAATGTGCCTTTCTCCCCTGATGGCAAACCTTGCACCCCACCTGCTCTTAATTGTT |
| | ATTGGCCATTAAATGATTATGGTTTTTACACCACTACTGGCATTGGCTACCAACCTTACAGAGTTGT |
| | AGTACTTTCTTTTGAACTTTTAAATGCACCGGCCACGGTTTGTGGACCAAAATTATCCACTGACCTT |
| | ATTAAGAACCAGTGTGTCAATTTTAATTTTAATGGACTCACTGGTACTGGTGTGTTAACTCCTTCTT |
| | CAAAGAGATTTCAACCATTTCAACAATTTGGCCGTGATGTTTCTGATTTCACTGATTCCGTTCGAGA |
| | TCCTAAAACATCTGAAATATTAGACATTTCACCTTGCTCTTTTGGGGGTGTAAGTGTAATTACACCT |
| | GGAACAAATGCTTCATCTGAAGTTGCTGTTCTATATCAAGATGTTAACTGCACTGATGTTTCTACAG |
| | CAATTCATGCAGATCAACTCACACCAGCTTGGCGCATATATTCTACTGGAAACAATGTATTCCAGAC |
| | TCAAGCAGGCTGTCTTATAGGAGCTGAGCATGTCGACACTTCTTATGAGTGCGACATTCCTATTGGA |
| | GCTGGCATTTGTGCTAGTTACCATACAGTTTCTTTATTACGTAGTACTAGCCAAAAATCTATTGTGG |
| | CTTATACTATGTCTTTAGGTGCTGATAGTTCAATTGCTTACTCTAATAACACCATTGCTATACCTAC |
| | TAACTTTTCAATTAGCATTACTACAGAAGTAATGCCTGTTTCTATGGCTAAAACCTCCGTAGATTGT |
| | AATATGTACATCTGCGGAGATTCTACTGAATGTGCTAATTTGCTTCTCCAATATGGTAGCTTTTGCA |
| | CACAACTAAATCGTGCACTCTCAGGTATTGCTGCTGAACAGGATCGCAACACACGTGAAGTGTTCGC |
| | TCAAGTCAAACAAATGTACAAAACCCCAACTTTGAAATATTTTGGTGGTTTTAATTTTTCACAAATA |
| | TTACCTGACCCTCTAAAGCCAACTAAGAGGTCTTTTATTGAGGACTTGCTCTTTAATAAGGTGACAC |
| | TCGCTGATGCTGGCTTCATGAAGCAATATGGCGAATGCCTAGGTGATATTAATGCTAGAGATCTCAT |
| | TTGTGCGCAGAAGTTCAATGGACTTACAGTGTTGCCACCTCTGCTCACTGATGATATGATTGCTGCC |
| | TACACTGCTGCTCTAGTTAGTGGTACTGCCACTGCTGGATGGACATTTGGTGCTGGCGCTGCTCTTC |
| | AAATACCTTTTGCTATGCAAATGGCATATAGGTTCAATGGCATTGGAGTTACCCAAAATGTTCTCTA |
| | TGAGAACCAAAAACAAATCGCCAACCAATTTAACAAGGCGATTAGTCAAATTCAAGAATCACTTACA |
| | ACAACATCAACTGCATTGGGCAAGCTGCAAGACGTTGTTAACCAGAATGCTCAAGCATTAAACACAC |
| | TTGTTAAACAACTTAGCTCTAATTTTGGTGCAATTTCAAGTGTGCTAAATGATATCCTTTCGCGACT |
| | TGATAAAGTCGAGGCGGAGGTACAAATTGACAGGTTAATTACAGGCAGACTTCAAAGCCTTCAAACC |
| | TATGTAACACAACAACTAATCAGGGCTGCTGAAATCAGGGCTTCTGCTAATCTTGCTGCTACTAAAA |
| | TGTCTGAGTGTGTTCTTGGACAATCAAAAAGAGTTGACTTTTGTGGAAAGGGCTACCACCTTATGTC |
| | CTTCCCACAAGCAGCCCCGCATGGTGTTGTCTTCCTACATGTCACGTATGTGCCATCCCAGGAGAGG |
| | AACTTCACCACAGCGCCAGCAATTTGTCATGAAGGCAAAGCATACTTCCCTCGTGAAGGTGTTTTTG |
| | TGTTTAATGGCACTTCTTGGTTTATTACACAGAGGAACTTCTTTTCTCCACAAATAATTACTACAGA |
| | CAATACATTTGTCTCAGGAAATTGTGATGTCGTTATTGGCATCATTAACAACACAGTTTATGATCCT |
| | CTGCAACCTGAGCTCGACTCATTCAAAGAAGAGCTGGACAAGTACTTCAAAAATCATACATCACCAG |
| | ATGTTGATCTTGGCGACATTTCAGGCATTAACGCTTCTGTCGTCAACATTCAAAAAGAAATTGACCG |
| | CCTCAATGAGGTCGCTAAAAATTTAAATGAATCACTCATTGACCTTCAAGAATTGGGAAAATATGAG |
| | CAATATATTAAATGGCCTTGGTATGTTTGGCTCGGCTTCATTGCTGGACTAATTGCCATCGTCATGG |
| | TTACAATCTTGCTTTGTTGCATGACTAGTTGTTGCAGTTGCCTCAAGGGTGCATGCTCTTGTGGTTC |
| | TTGCTGCAAGTTTGATGAGGATGACTCTGAGCCAGTTCTCAAGGGTGTCAAATTACATTACACATAA |
| | ACGAACTTATGGATTTGTTTATGAGATTTTTTACTCTTGGATCAATTACTGCACAGCCAGTAAAAAT |
| | TGACAATGCTTCTCCTGCAAGTACTGTTCATGCTACAGCAACGATACCGCTACAAGCCTCACTCCCT |
| | TTCGGATGGCTTGTTATTGGCGTTGCATTTCTTGCTGTTTTTCAGAGCGCTACCAAAATAATTGCGC |
| | TCAATAAAAGATGGCAGCTAGCCCTTTATAAGGGCTTCCAGTTCATTTGCAATTTACTGCTGCTATT |
| | TGTTACCATCTATTCACATCTTTTGCTTGTCGCTGCAGGTATGGAGGCGCAATTTTTGTACCTCTAT |
| | GCCTTGATATATTTTCTACAATGCATCAACGCATGTAGAATTATTATGAGATGTTGGCTTTGTTGGA |
| | AGTGCAAATCCAAGAACCCATTACTTTATGATGCCAACTACTTTGTTTGCTGGCACACACATAACTA |
| | TGACTACTGTATACCATATAACAGTGTCACAGATACAATTGTCGTTACTGAAGGTGACGGCATTTCA |
| | ACACCAAAACTCAAAGAAGACTACCAAATTGGTGGTTATTCTGAGGATAGGCACTCAGGTGTTAAAG |
| | ACTATGTCGTTGTACATGGCTATTTCACCGAAGTTTACTACCAGCTTGAGTCTACACAAATTACTAC |
| | AGACACTGGTATTGAAAATGCTACATTCTTCATCTTTAACAAGCTTGTTAAAGACCCACCGAATGTG |
| | CAAATACACACAATCGACGGCTCTTCAGGAGTTGCTAATCCAGCAATGGATCCAATTTATGATGAGC |
| | CGACGACGACTACTAGCGTGCCTTTGTAAGCACAAGAAAGTGAGTACGAACTTATGTACTCATTCGT |
| | TTCGGAAGAAACAGGTACGTTAATAGTTAATAGCGTACTTCTTTTTCTTGCTTTCGTGGTATTCTTG |
| | CTAGTCACACTAGCCATCCTTACTGCGCTTCGATTGTGTGCGTACTGCTGCAATATTGTTAACGTGA |
| | GTTTAGTAAAACCAACGGTTTACGTCTACTCGCGTGTTAAAAATCTGAACTCTTCTGAAGGAGTTCC |
| | TGATCTTCTGGTCTAAACGAACTAACTATTATTATTATTCTGTTTGGAACTTTAACATTGCTTATCA |
| | TGGCAGACAACGGTACTATTACCGTTGAGGAGCTTAAACAACTCCTGGAACAATGGAACCTAGTAAT |
| | AGGTTTCCTATTCCTAGCCTGGATTATGTTACTACAATTTGCCTATTCTAATCGGAACAGGTTTTTG |
| | TACATAATAAAGCTTGTTTTCCTCTGGCTCTTGTGGCCAGTAACACTTGCTTGTTTTGTGCTTGCTG |
| | CTGTCTACAGAATTAATTGGGTGACTGGCGGGATTGCGATTGCAATGGCTTGTATTGTAGGCTTGAT |
| | GTGGCTTAGCTACTTCGTTGCTTCCTTCAGGCTGTTTGCTCGTACCCGCTCAATGTGGTCATTCAAC |
| | CCAGAAACAAACATTCTTCTCAATGTGCCTCTCCGGGGGACAATTGTGACCAGACCGCTCATGGAAA |
| | GTGAACTTGTCATTGGTGCTGTGATCATTCGTGGTCACTTGCGAATGGCCGGACACCCCCTAGGGCG |
| | CTGTGACATTAAGGACCTGCCAAAAGAGATCACTGTGGCTACATCACGAACGCTTTCTTATTACAAA |
| | TTAGGAGCGTCGCAGCGTGTAGGCACTGATTCAGGTTTTGCTGCATACAACCGCTACCGTATTGGAA |
| | ACTATAAATTAAATACAGACCACGCCGGTAGCAACGACAATATTGCTTTGCTAGTACAGTAAGTGAC |
| | AACAGATGTTTCATCTTGTTGACTTCCAGGTTACAATAGCAGAGATATTGATTATCATTATGAGGAC |
| | TTTCAGGATTGCTATTTGGAATCTTGACGTTATAATAAGTTCAATAGTGAGACAATTATTTAAGCCT |
| | CTAACTAAGAAGAATTATTCGGAGTTAGATGATGAAGAACCTATGGAGTTAGATTATCCATAAAACG |
| | AACATGAAAATTATTCTCTTCCTGACATTGATTGTATTTACATCTTGCGAGCTATATCACTATCAGG |
| | AGTGTGTTAGAGGTACGACTGTACTACTAAAAGAACCTTGCCCATCAGGAACATACGAGGGCAATTC |
| | ACCATTTCACCCTCTTGCTGACAATAAATTTGCACTAACTTGCACTAGCACACACTTTGCTTTTGCT |
| | TGTGCTGACGGTACTCGACATACCTATCAGCTGCGTGCAAGATCAGTTTCACCAAAACTTTTCATCA |
| | GACAAGAGGAGGTTCAACAAGAGCTCTACTCGCCACTTTTTCTCATTGTTGCTGCTCTAGTATTTTT |
| | AATACTTTGCTTCACCATTAAGAGAAAGACAGAATGAATGAGCTCACTTTAATTGACTTCTATTTGT |
| | GCTTTTTAGCCTTTCTGCTATTCCTTGTTTTAATAATGCTTATTATATTTTGGTTTTCACTCGAAAT |
| | CCAGGATCTAGAAGAACCTTGTACCAAAGTCTAAACGAACATGAAACTTCTCATTGTTTTGACTTGT |
| | ATTTCTCTATGCAGTTGCATATGCACTGTAGTACAGCGCTGTGCATCTAATAAACCTCATGTGCTTG |
| | AAGATCCTTGTAAGGTACAACACTAGGGGTAATACTTATAGCACTGCTTGGCTTTGTGCTCTAGGAA |
| | AGGTTTTACCTTTTCATAGATGGCACACTATGGTTCAAACATGCACACCTAATGTTACTATCAACTG |
| | TCAAGATCCAGCTGGTGGTGCGCTTATAGCTAGGTGTTGGTACCTTCATGAAGGTCACCAAACTGCT |
| | GCATTTAGAGACGTACTTGTTGTTTTAAATAAACGAACAAATTAAAATGTCTGATAATGGACCCCAA |
| | TCAAACCAACGTAGTGCCCCCCGCATTACATTTGGTGGACCCACAGATTCAACTGACAATAACCAGA |
| | ATGGAGGACGCAATGGGGCAAGGCCAAAACAGCGCCGACCCCAAGGTTTACCCAATAATACTGCGTC |
| | TTGGTTCACAGCTCTCACTCAGCATGGCAAGGAGGAACTTAGATTCCCTCGAGGCCAGGGCGTTCCA |
| | ATCAACACCAATAGTGGTCCAGATGACCAAATTGGCTACTACCGAAGAGCTACCCGACGAGTTCGTG |
| | GTGGTGACGGCAAAATGAAAGAGCTCAGCCCCAGATGGTACTTCTATTACCTAGGAACTGGCCCAGA |
| | AGCTTCACTTCCCTACGGCGCTAACAAAGAAGGCATCGTATGGGTTGCAACTGAGGGAGCCTTGAAT |
| | ACACCCAAAGACCACATTGGCACCCGCAATCCTAATAACAATGCTGCCACCGTGCTACAACTTCCTC |
| | AAGGAACAACATTGCCAAAAGGCTTCTACGCAGAGGGAAGCAGAGGCGGCAGTCAAGCCTCTTCTCG |
| | CTCCTCATCACGTAGTCGCGGTAATTCAAGAAATTCAACTCCTGGCAGCAGTAGGGGAAATTCTCCT |
| | GCTCGAATGGCTAGCGGAGGTGGTGAAACTGCCCTCGCGCTATTGCTGCTAGACAGATTGAACCAGC |
| | TTGAGAGCAAAGTTTCTGGTAAAGGCCAACAACAACAAGGCCAAACTGTCACTAAGAAATCTGCTGC |
| | TGAGGCATCTAAAAAGCCTCGCCAAAAACGTACTGCCACAAAACAGTACAACGTCACTCAAGCATTT |
| | GGGAGACGTGGTCCAGAACAAACCCAAGGAAATTTCGGGGACCAAGACCTAATCAGACAAGGAACTG |
| | ATTACAAACATTGGCCGCAAATTGCACAATTTGCTCCAAGTGCCTCTGCATTCTTTGGAATGTCACG |
| | CATTGGCATGGAAGTCACACCTTCGGGAACATGGCTGACTTATCATGGAGCCATTAAATTGGATGAC |
| | AAAGATCCACAATTCAAAGACAACGTCATACTGCTGAACAAGCACATTGACGCATACAAAACATTCC |
| | CACCAACAGAGCCTAAAAAGGACAAAAAGAAAAAGACTGATGAAGCTCAGCCTTTGCCGCAGAGACA |
| | AAAGAAGCAGCCCACTGTGACTCTTCTTCCTGCGGCTGACATGGATGATTTCTCCAGACAACTTCAA |
| | AATTCCATGAGTGGAGCTTCTGCTGATTCAACTCAGGCATAAACACTCATGATGACCACACAAGGCA |
| | GATGGGCTATGTAAACGTTTTCGCAATTCCGTTTACGATACATAGTCTACTCTTGTGCAGAATGAAT |
| | TCTCGTAACTAAACAGCACAAGTAGGTTTAGTTAACTTTAATCTCACATAGCAATCTTTAATCAATG |
| | TGTAACATTAGGGAGGACTTGAAAGAGCCACCACATTTTCATCGAGGCCACGCGGAGTACGATCGAG |
| | GGTACAGTGAATAATGCTAGGGAGAGCTGCCTATATGGAAGAGCCCTAATGTGTAAAATTAATTTTA |
| | GTAGTGCTATCCCCATGTGATTTTAATAGCTTCTTAGGAGAATGAC |
| 3 | GenBank Accession No. NC_038294.1 (Betacoronavirus England 1 isolate H123990006, |
| | complete genome) |
| | ATTTAAGTGAATAGCTTGGCTATCTCACTTCCCCTCGTTCTCTTGCAGAACTTTGATTTTAACGAAC |
| | TTAAATAAAAGCCCTGTTGTTTAGCGTATTGTTGCACTTGTCTGGTGGGATTGTGGCATTAATTTGC |
| | CTGCTCATCTAGGCAGTGGACATATGCTCAACACTGGGTATAATTCTAATTGAATACTATTTTTCAG |
| | TTAGAGCGTCGTGTCTCTTGTACGTCTCGGTCACAATACACGGTTTCGTCCGGTGCGTGGCAATTCG |
| | GGGCACATCATGTCTTTCGTGGCTGGTGTGACCGCGCAAGGTGCGCGCGGTACGTATCGAGCAGCGC |
| | TCAACTCTGAAAAACATCAAGACCATGTGTCTCTAACTGTGCCACTCTGTGGTTCAGGAAACCTGGT |
| | TGAAAAACTTTCACCATGGTTCATGGATGGCGAAAATGCCTATGAAGTGGTGAAGGCCATGTTACTT |
| | AAAAAGGAGCCACTTCTCTATGTGCCCATCCGGCTGGCTGGACACACTAGACACCTCCCAGGTCCTC |
| | GTGTATACCTGGTTGAGAGGCTCATTGCTTGTGAAAATCCATTCATGGTTAACCAATTGGCTTATAG |
| | CTCTAGTGCAAATGGCAGCTTGGTTGGCACAACTTTGCAGGGCAAGCCTATTGGTATGTTCTTCCCT |
| | TATGACATCGAACTTGTCACAGGAAAGCAAAATATTCTCCTGCGCAAGTATGGCCGTGGTGGTTATC |
| | ACTACACCCCATTCCACTATGAGCGAGACAACACCTCTTGCCCTGAGTGGATGGACGATTTTGAGGC |
| | GGATCCTAAAGGCAAATATGCCCAGAATCTGCTTAAGAAGTTGATTGGCGGTGATGTCACTCCAGTT |
| | GACCAATACATGTGTGGCGTTGATGGAAAACCCATTAGTGCCTACGCATTTTTAATGGCCAAGGATG |
| | GAATAACCAAACTGGCTGATGTTGAAGCGGACGTCGCAGCACGTGCTGATGACGAAGGCTTCATCAC |
| | ATTAAAGAACAATCTATATAGATTGGTTTGGCATGTTGAGCGTAAAGACGTTCCATATCCTAAGCAA |
| | TCTATTTTTACTATTAATAGTGTGGTCCAAAAGGATGGTGTTGAAAACACTCCTCCTCACTATTTTA |
| | CTCTTGGATGCAAAATTTTAACGCTCACCCCACGCAACAAGTGGAGTGGCGTTTCTGACTTGTCCCT |
| | CAAACAAAAACTCCTTTACACCTTCTATGGTAAGGAGTCACTTGAGAACCCAACCTACATTTACCAC |
| | TCCGCATTCATTGAGTGTGGAAGTTGTGGTAATGATTCCTGGCTTACAGGGAATGCTATCCAAGGGT |
| | TTGCCTGTGGATGTGGGGCATCATATACAGCTAATGATGTCGAAGTCCAATCATCTGGCATGATTAA |
| | GCCAAATGCTCTTCTTTGTGCTACTTGCCCCTTTGCTAAGGGTGATAGCTGTTCTTCTAATTGCAAA |
| | CATTCAGTTGCTCAGTTGGTTAGTTACCTTTCTGAACGTTGTAATGTTATTGCTGATTCTAAGTCCT |
| | TCACACTTATCTTTGGTGGCGTAGCTTACGCCTACTTTGGATGTGAGGAAGGTACTATGTACTTTGT |
| | GCCTAGAGCTAAGTCTGTTGTCTCAAGGATTGGAGACTCCATCTTTACAGGCTGTACTGGCTCTTGG |
| | AACAAGGTCACTCAAATTGCTAACATGTTCTTGGAACAGACTCAGCATTCCCTTAACTTTGTGGGAG |
| | AGTTCGTTGTCAACGATGTTGTCCTCGCAATTCTCTCTGGAACCACAACTAATGTTGACAAAATACG |
| | CCAGCTTCTCAAAGGTGTCACCCTTGACAAGTTGCGTGATTATTTAGCTGACTATGACGTAGCAGTC |
| | ACTGCCGGCCCATTCATGGATAATGCTATTAATGTTGGTGGTACAGGATTACAGTATGCCGCCATTA |
| | CTGCACCTTATGTAGTTCTCACTGGCTTAGGTGAGTCCTTTAAGAAAGTTGCAACCATACCGTACAA |
| | GGTTTGCAACTCTGTTAAGGATACTCTGACTTATTATGCTCACAGCGTGTTGTACAGAGTTTTTCCT |
| | TATGACATGGATTCTGGTGTGTCATCCTTTAGTGAACTACTTTTTGATTGCGTTGATCTTTCAGTAG |
| | CTTCTACCTATTTTTTAGTCCGCCTCTTGCAAGATAAGACTGGCGACTTTATGTCTACAATTATTAC |
| | TTCCTGCCAAACTGCTGTTAGTAAGCTTCTAGATACATGTTTTGAAGCTACAGAAGCAACATTTAAC |
| | TTCTTGTTAGATTTGGCAGGATTGTTCAGAATCTTTCTTCGCAATGCCTATGTGTACACTTCACAAG |
| | GGTTTGTGGTGGTCAATGGCAAAGTTTCTACACTTGTCAAACAAGTGTTAGACTTGCTTAATAAGGG |
| | TATGCAACTTTTGCATACAAAGGTCTCCTGGGCTGGTTCTAATATCAGTGCTGTTATCTACAGCGGC |
| | AGGGAGTCTCTAATATTCCCATCGGGAACCTATTACTGTGTCACCACTAAGGCTAAGTCCGTTCAAC |
| | AAGATCTTGACGTTATTTTGCCTGGTGAGTTTTCCAAGAAGCAGTTAGGACTGCTCCAACCTACTGA |
| | CAATTCTACAACTGTTAGTGTTACTGTATCCAGTAACATGGTTGAAACTGTTGTGGGTCAACTTGAG |
| | CAAACTAATATGCATAGTCCTGATGTTATAGTAGGTGACTATGTCATTATTAGTGAAAAATTGTTTG |
| | TGCGTAGTAAGGAAGAAGACGGATTTGCCTTCTACCCTGCTTGCACTAATGGTCATGCTGTACCGAC |
| | TCTCTTTAGACTTAAGGGAGGTGCACCTGTAAAAAAAGTAGCCTTTGGCGGTGATCAAGTACATGAG |
| | GTTGCTGCTGTAAGAAGTGTTACTGTCGAGTACAACATTCATGCTGTATTAGACACACTACTTGCTT |
| | CTTCTAGTCTTAGAACCTTTGTTGTAGATAAGTCTTTGTCAATTGAGGAGTTTGCTGACGTAGTAAA |
| | GGAACAAGTCTCAGACTTGCTTGTTAAATTACTGCGTGGAATGCCGATTCCAGATTTTGATTTAGAC |
| | GATTTTATTGACGCACCATGCTATTGCTTTAACGCTGAGGGTGATGCATCTTGGTCTTCTACTATGA |
| | TCTTCTCTCTTCACCCCGTCGAGTGTGACGAGGAGTGTTCTGAAGTAGAGGCTTCAGATTTAGAAGA |
| | AGGTGAATCAGAGTGCATTTCTGAGACTTCAACTGAACAAGTTGACGTTTCTCATGAGATTTCTGAC |
| | GACGAGTGGGCTGCTGCAGTTGATGAAGCGTTCCCCCTCGATGAAGCAGAAGATGTTACTGAATCTG |
| | TGCAAGAAGAAGCACAACCAGTAGAAGTACCTGTTGAAGATATTGCGCAGGTTGTCATAGCTGACAC |
| | CTTACAGGAAACTCCTGTTGTGTCTGATACTGTTGAAGTCCCACCGCAAGTGGTGAAACTTCCGTCT |
| | GAACCTCAGACTATCCAGCCCGAGGTAAAAGAAGTTGCACCTGTCTATGAGGCTGATACCGAACAGA |
| | CACAGAGTGTTACTGTTAAACCTAAGAGGTTACGCAAAAAGCGTAATGTTGACCCTTTGTCCAATTT |
| | TGAACATAAGGTTATTACAGAGTGCGTTACCATAGTTTTAGGTGACGCAATTCAAGTAGCCAAGTGC |
| | TATGGGGAGTCTGTGTTAGTTAATGCTGCTAACACACATCTTAAGCATGGCGGTGGTATCGCTGGTG |
| | CTATTAATGCGGCTTCAAAAGGGGCTGTCCAAAAAGAGTCAGATGAGTATATTCTGGCTAAAGGGCC |
| | GTTACAAGTAGGAGATTCAGTTCTCTTGCAAGGCCATTCTCTAGCTAAGAATATCCTGCATGTCGTA |
| | GGCCCAGATGCCCGCGCTAAACAGGATGTTTCTCTCCTTAGTAAGTGCTATAAGGCTATGAATGCAT |
| | ATCCTCTTGTAGTCACTCCTCTTGTTTCAGCAGGCATATTTGGTGTAAAACCAGCTGTGTCTTTTGA |
| | TTATCTTATTAGAGAGGCTAAGACTAGAGTTTTAGTCGTCGTTAATTCCCAAGATGTCTATAAGAGT |
| | CTTACCATAGTTGACATTCCACAGAGTTTGACTTTTTCATATGATGGGTTACGTGGCGCAATACGTA |
| | AAGCTAAAGATTATGGTTTTACTGTTTTTGTGTGCACAGACAACTCTGCTAACACTAAAGTTCTTAG |
| | GAACAAGGGTGTTGATTATACTAAGAAGTTTCTTACAGTTGACGGTGTGCAATATTATTGCTACACG |
| | TCTAAGGACACTTTAGATGATATCTTACAACAGGCTAATAAGTCTGTTGGTATTATATCTATGCCTT |
| | TGGGATATGTGTCTCATGGTTTAGACTTAATTCAAGCAGGGAGTGTCGTGCGTAGAGTTAACGTGCC |
| | CTACGTGTGTCTCCTAGCTAATAAAGAGCAAGAAGCTATTTTGATGTCTGAAGACGTTAAGTTAAAC |
| | CCTTCAGAAGATTTTATAAAGCACGTCCGCACTAATGGTGGTTACAATTCTTGGCATTTAGTCGAGG |
| | GTGAACTATTGGTGCAAGACTTACGCTTAAATAAGCTCCTGCATTGGTCTGATCAAACCATATGCTA |
| | CAAGGATAGTGTGTTTTATGTTGTAAAGAATAGTACAGCTTTTCCATTTGAAACACTTTCAGCATGT |
| | CGTGCGTATTTGGATTCACGCACGACACAGCAGTTAACAATCGAAGTCTTAGTGACTGTCGATGGTG |
| | TAAATTTTAGAACAGTCGTTCTAAATAATAAGAACACTTATAGATCACAGCTTGGATGCGTTTTCTT |
| | TAATGGTGCTGATATTTCTGATACCATTCCTGATGAGAAACAGAATGGTCACAGTTTATATCTAGCA |
| | GACAATTTGACTGCTGATGAAACAAAGGCGCTTAAAGAGTTATATGGCCCCGTTGATCCTACTTTCT |
| | TACACAGATTCTATTCACTTAAGGCTGCAGTCCATAAGTGGAAGATGGTTGTGTGTGATAAGGTACG |
| | TTCTCTCAAATTGAGTGATAATAATTGTTATCTTAATGCAGTTATTATGACACTTGATTTATTGAAG |
| | GACATTAAATTTGTTATACCTGCTCTACAGCATGCATTTATGAAACATAAGGGCGGTGATTCAACTG |
| | ACTTCATAGCCCTCATTATGGCTTATGGCAATTGCACATTTGGTGCTCCAGATGATGCCTCTCGGTT |
| | ACTTCATACCGTGCTTGCAAAGGCTGAGTTATGCTGTTCTGCACGCATGGTTTGGAGAGAGTGGTGC |
| | AATGTCTGTGGCATAAAAGATGTTGTTCTACAAGGCTTAAAAGCTTGTTGTTACGTGGGTGTGCAAA |
| | CTGTTGAAGATCTGCGTGCTCGCATGACATATGTATGCCAGTGTGGTGGTGAACGTCATCGGCAAAT |
| | AGTCGAACACACCACCCCCTGGTTGCTGCTCTCAGGCACACCAAATGAAAAATTGGTGACAACCTCC |
| | ACGGCGCCTGATTTTGTAGCGTTTAATGTCTTTCAGGGCATTGAAACGGCTGTTGGCCATTATGTTC |
| | ATGCTCGCCTGAAGGGTGGTCTTATTTTAAAGTTTGACTCTGGCACCGTTAGCAAGACTTCAGACTG |
| | GAAGTGCAAGGTGACAGATGTACTTTTCCCCGGCCAAAAATACAGTAGCGATTGTAATGTCGTACGG |
| | TATTCTTTGGACGGTAATTTCAGAACAGAGGTTGATCCCGACCTATCTGCTTTCTATGTTAAGGATG |
| | GTAAATACTTTACAAGTGAACCACCCGTAACATATTCACCAGCTACAATTTTAGCTGGTAGTGTCTA |
| | CACTAATAGCTGCCTTGTATCGTCTGATGGACAACCTGGCGGTGATGCTATTAGTTTGAGTTTTAAT |
| | AACCTTTTAGGGTTTGATTCTAGTAAACCAGTCACTAAGAAATACACTTACTCCTTCTTGCCTAAAG |
| | AAGACGGCGATGTGTTGTTGGCTGAGTTTGACACTTATGACCCTATTTATAAGAATGGTGCCATGTA |
| | TAAAGGCAAACCAATTCTTTGGGTCAACAAAGCATCTTATGATACTAATCTTAATAAGTTCAATAGA |
| | GCTAGTTTGCGTCAAATTTTTGACGTAGCCCCCATTGAACTCGAAAATAAATTCACACCTTTGAGTG |
| | TGGAGTCTACACCAGTTGAACCTCCAACTGTAGATGTGGTAGCACTTCAACAGGAAATGACAATTGT |
| | CAAATGTAAGGGTTTAAATAAACCTTTCGTGAAGGACAATGTCAGTTTCGTTGCTGATGACTCAGGT |
| | ACTCCCGTTGTTGAGTATCTGTCTAAAGAAGATCTACATACATTGTATGTAGACCCTAAGTATCAAG |
| | TCATTGTCTTAAAAGACAATGTACTTTCTTCTATGCTTAGATTGCACACCGTTGAGTCAGGTGATAT |
| | TAACGTTGTTGCAGCTTCCGGATCTTTGACACGTAAAGTGAAGTTACTATTTAGGGCTTCATTTTAT |
| | TTCAAAGAATTTGCTACCCGCACTTTCACTGCTACCACTGCTGTAGGTAGTTGTATAAAGAGTGTAG |
| | TGCGGCATCTAGGTGTTACTAAAGGCATATTGACAGGCTGTTTTAGTTTTGTCAAGATGTTATTTAT |
| | GCTTCCACTAGCTTACTTTAGTGATTCAAAACTCGGCACCACAGAGGTTAAAGTGAGTGCTTTGAAA |
| | ACAGCTGGCGTTGTGACAGGTAATGTTGTAAAACAGTGTTGCACTGCTGCTGTTGATTTAAGTATGG |
| | ATAAGTTGCGCCGTGTGGATTGGAAATCAACCCTACGGTTGTTACTTATGTTATGCACAACTATGGT |
| | ATTGTTGTCTTCTGTGTATCACTTGTATGTCTTCAATCAGGTCTTATCAAGTGATGTTATGTTTGAA |
| | GATGCCCAAGGTTTGAAAAAGTTCTACAAAGAAGTTAGAGCTTACCTAGGAATCTCTTCTGCTTGTG |
| | ACGGTCTTGCTTCAGCTTATAGGGCGAATTCCTTTGATGTACCTACATTCTGCGCAAACCGTTCTGC |
| | AATGTGTAATTGGTGCTTGATTAGCCAAGATTCCATAACTCACTACCCAGCTCTTAAGATGGTTCAA |
| | ACACATCTTAGCCACTATGTTCTTAACATAGATTGGTTGTGGTTTGCATTTGAGACTGGTTTGGCAT |
| | ACATGCTCTATACCTCGGCCTTCAACTGGTTGTTGTTGGCAGGTACATTGCATTATTTCTTTGCACA |
| | GACTTCCATATTTGTAGACTGGCGGTCATACAATTATGCTGTGTCTAGTGCCTTCTGGTTATTCACC |
| | CACATTCCAATGGCGGGTTTGGTACGAATGTATAATTTGTTAGCATGCCTTTGGCTTTTACGCAAGT |
| | TTTATCAGCATGTAATCAATGGTTGCAAAGATACGGCATGCTTGCTCTGCTATAAGAGGAACCGACT |
| | TACTAGAGTTGAAGCTTCTACCGTTGTCTGTGGTGGAAAACGTACGTTTTATATCACAGCAAATGGC |
| | GGTATTTCATTCTGTCGTAGGCATAATTGGAATTGTGTGGATTGTGACACTGCAGGTGTGGGGAATA |
| | CCTTCATCTGTGAAGAAGTCGCAAATGACCTCACTACCGCCCTACGCAGGCCTATTAACGCTACGGA |
| | TAGATCACATTATTATGTGGATTCCGTTACAGTTAAAGAGACTGTTGTTCAGTTTAATTATCGTAGA |
| | GACGGTCAACCATTCTACGAGCGGTTTCCCCTCTGCGCTTTTACAAATCTAGATAAGTTGAAGTTCA |
| | AAGAGGTCTGTAAAACTACTACTGGTATACCTGAATACAACTTTATCATCTACGACTCATCAGATCG |
| | TGGCCAGGAAAGTTTAGCTAGGTCTGCATGTGTTTATTATTCTCAAGTCTTGTGTAAATCAATTCTT |
| | TTGGTTGACTCAAGTTTGGTTACTTCTGTTGGTGATTCTAGTGAAATCGCCACTAAAATGTTTGATT |
| | CCTTTGTTAATAGTTTCGTCTCGCTGTATAATGTCACACGCGATAAGTTGGAAAAACTTATCTCTAC |
| | TGCTCGTGATGGCGTAAGGCGAGGCGATAACTTCCATAGTGTCTTAACAACATTCATTGACGCAGCA |
| | CGAGGCCCCGCAGGTGTGGAGTCTGATGTTGAGACCAATGAAATTGTTGACTCTGTGCAGTATGCTC |
| | ATAAACATGACATACAAATTACTAATGAGAGTTACAATAATTATGTACCCTCATATGTTAAACCTGA |
| | TAGTGTGTCTACCAGTGATTTAGGTAGTCTCATTGATTGTAATGCGGCTTCAGTTAACCAAATTGTC |
| | TTGCGTAATTCTAATGGTGCTTGTATTTGGAACGCTGCTGCATATATGAAACTCTCGGATGCACTTA |
| | AACGACAGATTCGCATTGCATGCCGTAAGTGTAATTTAGCTTTCCGGTTAACCACCTCAAAGCTACG |
| | CGCTAATGATAATATCTTATCAGTTAGATTCACTGCTAACAAAATTGTTGGTGGTGCTCCTACATGG |
| | TTTAATGCGTTGCGTGACTTTACGTTAAAGGGTTACGTTCTTGCTACCATTATTGTGTTTCTGTGTG |
| | CTGTACTGATGTATTTGTGTTTACCTACATTTTCTATGGTACCTGTTGAATTTTATGAAGACCGCAT |
| | CTTGGACTTTAAAGTTCTTGATAATGGTATCATTAGGGATGTAAATCCTGATGATAAGTGCTTTGCT |
| | AATAAGCACCGGTCCTTCACACAATGGTATCATGAGCATGTTGGTGGTGTCTATGACAACTCTATCA |
| | CATGCCCATTGACAGTTGCAGTAATTGCTGGAGTTGCTGGTGCTCGCATTCCAGACGTACCTACTAC |
| | ATTGGCTTGGGTGAACAATCAGATAATTTTCTTTGTTTCTCGAGTCTTTGCTAATACAGGCAGTGTT |
| | TGCTACACTCCTATAGATGAGATACCCTATAAGAGTTTCTCTGATAGTGGTTGCATTCTTCCATCTG |
| | AGTGCACTATGTTTAGGGATGCAGAGGGCCGTATGACACCATACTGCCATGATCCTACTGTTTTGCC |
| | TGGGGCTTTTGCGTACAGTCAGATGAGGCCTCATGTTCGTTACGACTTGTATGATGGTAACATGTTT |
| | ATTAAATTTCCTGAAGTAGTATTTGAAAGTACACTTAGGATTACTAGAACTCTGTCAACTCAGTACT |
| | GCCGGTTCGGTAGTTGTGAGTATGCACAAGAGGGTGTTTGTATTACCACAAATGGCTCGTGGGCCAT |
| | TTTTAATGACCACCATCTTAATAGACCTGGTGTCTATTGTGGCTCTGATTTTATTGACATTGTCAGG |
| | CGGTTAGCAGTATCACTGTTCCAGCCTATTACTTATTTCCAATTGACTACCTCATTGGTCTTGGGTA |
| | TAGGTTTGTGTGCGTTCCTGACTTTGCTCTTCTATTATATTAATAAAGTAAAACGTGCTTTTGCAGA |
| | TTACACCCAGTGTGCTGTAATTGCTGTTGTTGCTGCTGTTCTTAATAGCTTGTGCATCTGCTTTGTT |
| | GCCTCTATACCATTGTGTATAGTACCTTACACTGCATTGTACTATTATGCTACATTCTATTTTACTA |
| | ATGAGCCTGCATTTATTATGCATGTTTCTTGGTACATTATGTTCGGGCCTATCGTTCCCATATGGAT |
| | GACCTGCGTCTATACAGTTGCAATGTGCTTTAGACACTTCTTCTGGGTTTTAGCTTATTTTAGTAAG |
| | AAACATGTAGAAGTTTTTACTGATGGTAAGCTTAATTGTAGTTTCCAGGACGCTGCCTCTAATATCT |
| | TTGTTATTAACAAGGACACTTATGCAGCTCTTAGAAACTCTTTAACTAATGATGCCTATTCACGATT |
| | TTTGGGGTTGTTTAACAAGTATAAGTACTTCTCTGGTGCTATGGAAACAGCCGCTTATCGTGAAGCT |
| | GCAGCATGTCATCTTGCTAAAGCCTTACAAACATACAGCGAGACTGGTAGTGATCTTCTTTACCAAC |
| | CACCCAACTGTAGCATAACCTCTGGCGTGTTGCAAAGCGGTTTGGTGAAAATGTCACATCCCAGTGG |
| | AGATGTTGAGGCTTGTATGGTTCAGGTTACCTGCGGTAGCATGACTCTTAATGGTCTTTGGCTTGAC |
| | AACACAGTCTGGTGCCCACGACACGTAATGTGCCCGGCTGACCAGTTGTCTGATCCTAATTATGATG |
| | CCTTGTTGATTTCTATGACTAATCATAGTTTCAGTGTGCAAAAACACATTGGCGCTCCAGCAAACTT |
| | GCGTGTTGTTGGTCATGCCATGCAAGGCACTCTTTTGAAGTTGACTGTCGATGTTGCTAACCCTAGC |
| | ACTCCAGCCTACACTTTTACAACAGTGAAACCTGGCGCAGCATTTAGTGTGTTAGCATGCTATAATG |
| | GTCGTCCGACTGGTACATTCACTGTTGTAATGCGCCCTAACTACACAATTAAGGGTTCCTTTCTGTG |
| | TGGTTCTTGTGGTAGTGTTGGTTACACCAAGGAGGGTAGTGTGATCAATTTTTGTTACATGCATCAA |
| | ATGGAACTTGCTAATGGTACACATACCGGTTCAGCATTTGATGGTACTATGTATGGTGCCTTTATGG |
| | ATAAACAAGTGCACCAAGTTCAGTTAACAGACAAATACTGCAGTGTTAATGTAGTAGCTTGGCTTTA |
| | CGCAGCAATACTTAATGGTTGCGCTTGGTTTGTAAAACCTAATCGCACTAGTGTTGTTTCTTTTAAT |
| | GAATGGGCTCTTGCCAACCAATTCACTGAATTTGTTGGCACTCAATCCGTTGACATGTTAGCTGTCA |
| | AAACAGGCGTTGCTATTGAACAGCTGCTTTATGCGATCCAACAACTTTATACTGGGTTCCAGGGAAA |
| | GCAAATCCTTGGCAGTACCATGTTGGAAGATGAATTCACACCTGAGGATGTTAATATGCAGATTATG |
| | GGTGTGGTTATGCAGAGTGGTGTGAGAAAAGTTACATATGGTACTGCGCATTGGTTGTTCGCGACCC |
| | TTGTCTCAACCTATGTGATAATCTTACAAGCCACTAAATTTACTTTGTGGAACTACTTGTTTGAGAC |
| | TATTCCCACACAGTTGTTCCCACTCTTATTTGTGACTATGGCCTTCGTTATGTTGTTGGTTAAACAC |
| | AAACACACCTTTTTGACACTTTTCTTGTTGCCTGTGGCTATTTGTTTGACTTATGCAAACATAGTCT |
| | ACGAGCCCACTACTCCCATTTCGTCAGCGCTGATTGCAGTTGCAAATTGGCTTGCCCCCACTAATGC |
| | TTATATGCGCACTACACATACTGATATTGGTGTCTACATTAGTATGTCACTTGTATTAGTCATTGTA |
| | GTGAAGAGATTGTACAACCCATCACTTTCTAACTTTGCGTTAGCATTGTGCAGTGGTGTAATGTGGT |
| | TGTACACTTATAGCATTGGAGAAGCCTCAAGCCCCATTGCCTATCTGGTTTTTGTCACTACACTCAC |
| | TAGTGATTATACGATTACAGTCTTTGTTACTGTTAACCTTGCAAAAGTTTGCACTTATGCCATCTTT |
| | GCTTACTCGCCACAGCTTACACTTGTGTTTCCGGAAGTGAAGATGATACTTTTATTATACACATGTT |
| | TAGGTTTCATGTGTACTTGCTATTTTGGTGTCTTCTCTCTTTTGAACCTTAAGCTTAGAGCACCTAT |
| | GGGTGTCTATGACTTTAAGGTCTCAACACAAGAGTTCAGATTCATGACAGCTAACAATCTAACTGCA |
| | CCTAGAAATTCTTGGGAGGCTATGGCTCTGAACTTTAAGTTAATAGGTATTGGCGGTACACCTTGTA |
| | TAAAGGTTGCTGCTATGCAGTCTAAACTTACAGATCTTAAATGCACATCTGTGGTTCTCCTCTCTGT |
| | GCTCCAACAGTTACACTTAGAGGCTAATAGTAGGGCCTGGGCTTTCTGTGTTAAATGCCATAATGAT |
| | ATATTGGCAGCAACAGACCCCAGTGAGGCTTTCGAGAAATTCGTAAGTCTCTTTGCCACTTTAATGA |
| | CTTTTTCTGGTAATGTAGATCTTGATGCGTTAGCTAGTGATATTTTTGACACTCCTAGCGTACTTCA |
| | AGCTACTCTTTCTGAGTTTTCACACTTAGCTACCTTTGCTGAGTTGGAAGCTGCGCAGAAAGCCTAT |
| | CAGGAAGCTATGGACTCTGGTGACACCTCACCACAAGTTCTTAAGGCTTTGCAGAAGGCTGTTAATA |
| | TAGCTAAAAACGCCTATGAGAAGGATAAGGCAGTGGCCCGTAAGTTAGAACGTATGGCTGATCAGGC |
| | TATGACTTCTATGTATAAGCAAGCACGTGCTGAAGACAAGAAAGCAAAAATTGTCAGTGCTATGCAA |
| | ACTATGTTGTTTGGTATGATTAAGAAGCTCGACAACGATGTTCTTAATGGTATCATTTCTAACGCTA |
| | GGAATGGTTGTATACCTCTTAGTGTCATTCCACTGTGTGCTTCAAATAAACTTCGCGTTGTAATTCC |
| | TGACTTCACCGTCTGGAATCAGGTAGTCACATATCCCTCGCTTAACTACGCTGGGGCTTTGTGGGAC |
| | ATTACAGTTATAAACAATGTGGACAATGAAATTGTTAAGTCTTCAGATGTTGTAGACAGCAATGAAA |
| | ATTTAACATGGCCACTTGTTTTAGAATGCACTAGGGCATCCACTTCTGCCGTTAAGTTGCAAAATAA |
| | TGAGATCAAACCTTCAGGTTTAAAAACCATGGTTGTGTCTGCAGGTCAAGAGCAAACTAACTGTAAT |
| | ACTAGTTCCTTAGCTTATTACGAACCTGTGCAGGGTCGTAAAATGCTGATGGCTCTTCTTTCTGATA |
| | ATGCCTATCTCAAATGGGCGCGTGTTGAAGGTAAGGACGGATTTGTTAGTGTAGAGCTACAACCTCC |
| | TTGCAAATTCTTGATTGCGGGACCAAAAGGACCTGAAATCCGATATCTCTATTTTGTTAAAAATCTT |
| | AACAACCTTCATCGCGGGCAAGTGTTAGGGCACATTGCTGCGACTGTTAGATTGCAAGCTGGTTCTA |
| | ACACCGAGTTTGCCTCTAATTCTTCGGTGTTGTCACTTGTTAACTTCACCGTTGATCCTCAAAAAGC |
| | TTATCTCGATTTCGTCAATGCGGGAGGTGCCCCATTGACAAATTGTGTTAAGATGCTTACTCCTAAA |
| | ACTGGTACAGGTATAGCTATATCTGTTAAACCAGAGAGTACAGCTGATCAAGAGACTTATGGTGGAG |
| | CTTCAGTGTGTCTCTATTGCCGTGCGCATATAGAACATCCTGATGTCTCTGGTGTTTGTAAATATAA |
| | GGGTAAGTTTGTCCAAATCCCTGCTCAGTGTGTCCGTGACCCTGTGGGATTTTGTTTGTCAAATACC |
| | CCCTGTAATGTCTGTCAATATTGGATTGGATATGGGTGCAATTGTGACTCGCTTAGGCAAGCAGCAC |
| | TGCCCCAATCTAAAGATTCCAATTTTTTAAACGAGTCCGGGGTTCTATTGTAAATGCCCGAATAGAA |
| | CCCTGTTCAAGTGGTTTGTCCACTGATGTCGTCTTTAGGGCATTTGACATCTGCAACTATAAGGCTA |
| | AGGTTGCTGGTATTGGAAAATACTACAAGACTAATACTTGTAGGTTTGTAGAATTAGATGACCAAGG |
| | GCATCATTTAGACTCCTATTTTGTCGTTAAGAGGCATACTATGGAGAATTATGAACTAGAGAAGCAC |
| | TGTTACGATTTGTTACGTGACTGTGATGCTGTAGCTCCCCATGATTTCTTCATCTTTGATGTAGACA |
| | AAGTTAAAACACCTCATATTGTACGTCAGCGTTTAACTGAGTACACTATGATGGATCTTGTATATGC |
| | CCTGAGGCACTTTGATCAAAATAGCGAAGTGCTTAAGGCTATCTTAGTGAAGTATGGTTGCTGTGAT |
| | GTTACCTACTTTGAAAATAAACTCTGGTTTGATTTTGTTGAAAATCCCAGTGTTATTGGTGTTTATC |
| | ATAAACTTGGAGAACGTGTACGCCAAGCTATCTTAAACACTGTTAAATTTTGTGACCACATGGTCAA |
| | GGCTGGTTTAGTCGGTGTGCTCACACTAGACAACCAGGACCTTAATGGCAAGTGGTATGATTTTGGT |
| | GACTTCGTAATCACTCAACCTGGTTCAGGAGTAGCTATAGTTGATAGCTACTATTCTTATTTGATGC |
| | CTGTGCTCTCAATGACCGATTGTCTGGCCGCTGAGACACATAGGGATTGTGATTTTAATAAACCACT |
| | CATTGAGTGGCCACTTACTGAGTATGATTTTACTGATTATAAGGTACAACTCTTTGAGAAGTACTTT |
| | AAATATTGGGATCAGACGTATCACGCAAATTGCGTTAATTGTACTGATGACCGTTGTGTGTTACATT |
| | GTGCTAATTTCAATGTATTGTTTGCTATGACCATGCCTAAGACTTGTTTCGGACCCATAGTCCGAAA |
| | GATCTTTGTTGATGGCGTGCCATTTGTAGTATCTTGTGGTTATCACTACAAAGAATTAGGTTTAGTC |
| | ATGAATATGGATGTTAGTCTCCATAGACATAGGCTCTCTCTTAAGGAGTTGATGATGTATGCCGCTG |
| | ATCCAGCCATGCACATTGCCTCCTCTAACGCTTTTCTTGATTTGAGGACATCATGTTTTAGTGTCGC |
| | TGCACTTACAACTGGTTTGACTTTTCAAACTGTGCGGCCTGGCAATTTTAACCAAGACTTCTATGAT |
| | TTCGTGGTATCTAAAGGTTTCTTTAAGGAGGGCTCTTCAGTGACGCTCAAACATTTTTTCTTTGCTC |
| | AAGATGGTAATGCTGCTATTACAGATTATAATTACTATTCTTATAATCTGCCTACTATGTGTGACAT |
| | CAAACAAATGTTGTTCTGCATGGAAGTTGTAAACAAGTACTTCGAAATCTATGACGGTGGTTGTCTT |
| | AATGCTTCTGAAGTGGTTGTTAATAATTTAGACAAGAGTGCTGGCCATCCTTTTAATAAGTTTGGCA |
| | AAGCTCGTGTCTATTATGAGAGCATGTCTTACCAGGAGCAAGATGAACTCTTTGCCATGACAAAGCG |
| | TAACGTCATTCCTACCATGACTCAAATGAATCTAAAATATGCTATTAGTGCTAAGAATAGAGCTCGC |
| | ACTGTTGCAGGCGTGTCCATACTTAGCACAATGACTAATCGCCAGTACCATCAGAAAATGCTTAAGT |
| | CCATGGCTGCAACTCGTGGAGCGACTTGCGTCATTGGTACTACAAAGTTCTATGGTGGCTGGGATTT |
| | CATGCTTAAAACATTGTACAAAGATGTTGATAATCCGCATCTTATGGGTTGGGATTACCCTAAGTGT |
| | GATAGAGCTATGCCTAATATGTGTAGAATCTTCGCTTCACTCATATTAGCTCGTAAACATGGCACTT |
| | GTTGTACTACAAGGGACAGATTTTATCGCTTGGCAAATGAGTGTGCTCAGGTGCTAAGCGAATATGT |
| | TCTATGTGGTGGTGGTTACTACGTCAAACCTGGAGGTACCAGTAGCGGAGATGCCACCACTGCATAT |
| | GCCAATAGTGTCTTTAACATTTTGCAGGCGACAACTGCTAATGTCAGTGCACTTATGGGTGCTAATG |
| | GCAACAAGATTGTTGACAAAGAAGTTAAAGACATGCAGTTTGATTTGTATGTCAATGTTTACAGGAG |
| | CACTAGCCCAGACCCCAAATTTGTTGATAAATACTATGCTTTTCTTAATAAGCACTTTTCTATGATG |
| | ATACTGTCTGATGACGGTGTCGTTTGCTATAATAGTGATTATGCAGCTAAGGGTTACATTGCTGGAA |
| | TACAGAATTTCAAGGAAACGCTGTATTATCAGAACAATGTCTTTATGTCTGAAGCTAAATGCTGGGT |
| | GGAAACCGATCTGAAGAAAGGGCCACATGAATTCTGTTCACAGCATACGCTTTATATTAAGGATGGC |
| | GACGATGGTTACTTCCTTCCTTATCCAGACCCTTCAAGAATTTTGTCTGCCGGTTGCTTTGTAGATG |
| | ATATCGTTAAGACTGACGGTACACTCATGGTAGAGCGGTTTGTGTCTTTGGCTATAGATGCTTACCC |
| | TCTCACAAAGCATGAAGATATAGAATACCAGAATGTATTCTGGGTCTACTTACAGTATATAGAAAAA |
| | CTGTATAAAGACCTTACAGGACACATGCTTGACAGTTATTCTGTCATGCTATGTGGTGATAATTCTG |
| | CTAAGTTTTGGGAAGAGGCATTCTATAGAGATCTCTATAGTTCGCCTACCACTTTGCAGGCTGTCGG |
| | TTCATGCGTTGTATGCCATTCACAGACTTCCCTACGCTGTGGGACATGCATCCGTAGACCATTTCTC |
| | TGCTGTAAATGCTGCTATGATCATGTTATAGCAACTCCACATAAGATGGTTTTGTCTGTTTCTCCTT |
| | ACGTTTGTAATGCCCCTGGTTGTGGCGTTTCAGACGTTACTAAGCTATATTTAGGTGGTATGAGCTA |
| | CTTTTGTGTAGATCATAGACCTGTGTGTAGTTTTCCACTTTGCGCTAATGGTCTTGTATTCGGCTTA |
| | TACAAGAATATGTGCACAGGTAGTCCTTCTATAGTTGAATTTAATAGGTTGGCTACCTGTGACTGGA |
| | CTGAAAGTGGTGATTACACCCTTGCCAATACTACAACAGAACCACTCAAACTTTTTGCTGCTGAGAC |
| | TTTACGTGCCACTGAAGAGGCGTCTAAGCAGTCTTATGCTATTGCCACCATCAAAGAAATTGTTGGT |
| | GAGCGCCAACTATTACTTGTGTGGGAGGCTGGCAAGTCCAAACCACCACTCAATCGTAATTATGTTT |
| | TTACTGGTTATCATATAACCAAAAATAGTAAAGTGCAGCTCGGTGAGTACATCTTCGAGCGCATTGA |
| | TTATAGTGATGCTGTATCCTACAAGTCTAGTACAACGTATAAACTGACTGTAGGTGACATCTTCGTA |
| | CTTACCTCTCACTCTGTGGCTACCTTGACGGCGCCCACAATTGTGAATCAAGAGAGGTATGTTAAAA |
| | TTACTGGGTTGTACCCAACCATTACGGTACCTGAAGAGTTCGCAAGTCATGTTGCCAACTTCCAAAA |
| | ATCAGGTTATAGTAAATATGTCACTGTTCAGGGACCACCTGGCACTGGCAAAAGTCATTTTGCTATA |
| | GGGTTAGCGATTTACTACCCTACAGCACGTGTTGTTTATACAGCATGTTCACACGCAGCTGTTGATG |
| | CTTTGTGTGAAAAAGCTTTTAAATATTTGAACATTGCTAAATGTTCCCGTATCATTCCTGCAAAGGC |
| | ACGTGTTGAGTGCTATGACAGGTTTAAAGTTAATGAGACAAATTCTCAATATTTGTTTAGTACTATT |
| | AATGCTCTACCAGAAACTTCTGCCGATATTCTGGTGGTTGATGAGGTTAGTATGTGCACTAATTATG |
| | ATCTTTCAATTATTAATGCACGTATTAAAGCTAAGCACATTGTCTATGTAGGAGATCCAGCACAGTT |
| | GCCAGCTCCTAGGACTTTGTTGACTAGAGGCACATTGGAACCAGAAAATTTCAATAGTGTCACTAGA |
| | TTGATGTGTAACTTAGGTCCTGACATATTTTTAAGTATGTGCTACAGGTGTCCTAAGGAAATAGTAA |
| | GCACTGTGAGCGCTCTTGTCTACAATAATAAATTGTTAGCCAAGAAGGAGCTTTCAGGCCAGTGCTT |
| | TAAAATACTCTATAAGGGCAATGTGACGCATGATGCTAGCTCTGCCATTAATAGACCACAACTCACA |
| | TTTGTGAAGAATTTTATTACTGCCAATCCGGCATGGAGTAAGGCAGTCTTTATTTCGCCTTATAATT |
| | CACAGAATGCTGTGGCTCGTTCAATGCTGGGTCTTACTACTCAGACTGTTGATTCCTCACAGGGTTC |
| | AGAATACCAGTACGTTATCTTCTGTCAAACAGCAGATACGGCACATGCTAACAACATTAACAGATTT |
| | AATGTTGCAATCACTCGTGCCCAAAAAGGTATTCTTTGTGTTATGACATCTCAGGCACTCTTTGAGT |
| | CCTTAGAGTTTACTGAATTGTCTTTTACTAATTACAAGCTCCAGTCTCAGATTGTAACTGGCCTTTT |
| | TAAAGATTGCTCTAGAGAAACTTCTGGCCTCTCACCTGCTTATGCACCAACATACGTTAGTGTTGAT |
| | GACAAGTATAAGACGAGTGATGAGCTTTGCGTGAATCTTAATTTACCCGCAAATGTCCCATACTCTC |
| | GTGTTATTTCCAGGATGGGCTTTAAACTCGATGCAACAGTTCCTGGATATCCTAAGCTTTTCATTAC |
| | TCGTGAAGAGGCTGTAAGGCAAGTTCGAAGCTGGATAGGCTTCGATGTTGAGGGTGCTCATGCTTCC |
| | CGTAATGCATGTGGCACCAATGTGCCTCTACAATTAGGATTTTCAACTGGTGTGAACTTTGTTGTTC |
| | AGCCAGTTGGTGTTGTAGACACTGAGTGGGGTAACATGTTAACGGGCATTGCTGCCCGTCCTCCACC |
| | AGGTGAACAGTTTAAGCACCTCGTGCCTCTTATGCATAAGGGGGCTGCGTGGCCTATTGTTAGACGA |
| | CGTATAGTGCAAATGTTGTCAGACACTTTAGACAAATTGTCTGATTACTGTACGTTTGTTTGTTGGG |
| | CTCATGGCTTTGAATTAACGTCTGCATCATACTTTTGCAAGATAGGTAAGGAACAGAAGTGTTGCAT |
| | GTGCAATAGACGCGCTGCAGCGTACTCTTCACCTCTGCAATCTTATGCCTGCTGGACTCATTCCTGC |
| | GGTTATGATTATGTCTACAACCCTTTCTTTGTCGATGTTCAACAGTGGGGTTATGTAGGCAATCTTG |
| | CTACTAATCACGATCGTTATTGCTCTGTCCATCAAGGAGCTCATGTGGCTTCTAATGATGCAATAAT |
| | GACTCGTTGTTTAGCTATTCATTCTTGTTTTATAGAACGTGTGGATTGGGATATAGAGTATCCTTAT |
| | ATCTCACATGAAAAGAAATTGAATTCCTGTTGTAGAATCGTTGAGCGCAACGTCGTACGTGCTGCTC |
| | TTCTTGCCGGTTCATTTGACAAAGTCTATGATATTGGCAATCCTAAAGGAATTCCTATTGTTGATGA |
| | CCCTGTGGTTGATTGGCATTATTTTGATGCACAGCCCTTGACCAGAAAGGTACAACAGCTTTTCTAT |
| | ACAGAGGACATGGCCTCAAGATTTGCTGATGGGCTCTGCTTATTTTGGAACTGTAATGTACCAAAAT |
| | ATCCTAATAATGCAATTGTATGCAGGTTTGACACACGTGTGCATTCTGAGTTCAATTTGCCAGGTTG |
| | TGATGGCGGTAGTTTGTATGTTAACAAGCACGCTTTTCATACACCAGCATATGATGTGAGTGCATTC |
| | CGTGATCTGAAACCTTTACCATTCTTTTATTATTCTACTACACCATGTGAAGTGCATGGTAATGGTA |
| | GTATGATAGAGGATATTGATTATGTACCCCTAAAATCTGCAGTCTGTATTACAGCTTGTAATTTAGG |
| | GGGCGCTGTTTGTAGGAAGCATGCTACAGAGTACAGAGAGTATATGGAAGCATATAATCTTGTCTCT |
| | GCATCAGGTTTCCGCCTTTGGTGTTATAAGACCTTTGATATTTATAATCTCTGGTCTACTTTTACAA |
| | AAGTTCAAGGTTTGGAAAACATTGCTTTTAATTTTGTTAAACAAGGCCATTTTATTGGTGTTGAGGG |
| | TGAACTACCTGTAGCTGTAGTCAATGATAAGATCTTCACCAAGAGTGGCGTTAATGACATTTGTATG |
| | TTTGAGAATAAAACCACTTTGCCTACTAATATAGCTTTTGAACTCTATGCTAAGCGTGCTGTACGCT |
| | CGCATCCCGATTTCAAATTGCTACACAATTTACAAGCAGACATTTGCTACAAGTTCGTCCTTTGGGA |
| | TTATGAACGTAGCAATATTTATGGTACTGCTACTATTGGTGTATGTAAGTACACTGATATTGATGTT |
| | AATTCAGCTTTGAATATATGTTTTGACATACGCGATAATGGTTCATTGGAGAAGTTCATGTCTACTC |
| | CCAATGCCATCTTTATTTCTGATAGAAAAATCAAGAAATACCCTTGTATGGTAGGTCCTGATTATGC |
| | TTACTTCAATGGTGCTATCATCCGTGATAGTGATGTTGTTAAACAACCAGTGAAGTTCTACTTGTAT |
| | AAGAAAGTCAATAATGAGTTTATTGATCCTACTGAGTGTATTTACACTCAGAGTCGCTCTTGTAGTG |
| | ACTTCCTACCCCTGTCTGACATGGAGAAAGACTTTCTATCTTTTGATAGTGATGTTTTCATTAAGAA |
| | GTATGGCTTGGAAAACTATGCTTTTGAGCACGTAGTCTATGGAGACTTCTCTCATACTACGTTAGGC |
| | GGTCTTCACTTGCTTATTGGTTTATACAAGAAGCAACAGGAAGGTCATATTATTATGGAAGAAATGC |
| | TAAAAGGTAGCTCAACTATTCATAACTATTTTATTACTGAGACTAACACAGCGGCTTTTAAGGCGGT |
| | GTGTTCTGTTATAGATTTAAAGCTTGACGACTTTGTTATGATTTTAAAGAGTCAAGACCTTGGCGTA |
| | GTATCCAAGGTTGTCAAGGTTCCTATTGACTTAACAATGATTGAGTTTATGTTATGGTGTAAGGATG |
| | GACAGGTTCAAACCTTCTACCCTCGACTCCAGGCTTCTGCAGATTGGAAACCTGGTCATGCAATGCC |
| | ATCCCTCTTTAAAGTTCAAAATGTAAACCTTGAACGTTGTGAGCTTGCTAATTACAAGCAATCTATT |
| | CCTATGCCTCGCGGTGTGCACATGAACATCGCTAAATATATGCAATTGTGCCAGTATTTAAATACTT |
| | GCACATTAGCCGTGCCTGCCAATATGCGTGTTATACATTTTGGCGCTGGTTCTGATAAAGGTATCGC |
| | TCCTGGTACATCAGTTTTACGACAGTGGCTTCCTACAGATGCCATTATTATAGATAATGATTTAAAT |
| | GAGTTCGTGTCAGATGCTGACATAACTTTATTTGGAGATTGTGTAACTGTACGTGTCGGCCAACAAG |
| | TGGATCTTGTTATTTCCGACATGTATGATCCTACTACTAAGAATGTAACAGGTAGTAATGAGTCAAA |
| | GGCTTTATTCTTTACTTACCTGTGTAACCTCATTAATAATAATCTTGCTCTTGGTGGGTCTGTTGCT |
| | ATTAAAATAACAGAACACTCTTGGAGCGTTGAACTTTATGAACTTATGGGAAAATTTGCTTGGTGGA |
| | CTGTTTTCTGCACCAATGCAAATGCATCCTCATCTGAAGGATTCCTCTTAGGTATTAATTACTTGGG |
| | TACTATTAAAGAAAATATAGATGGTGGTGCTATGCACGCCAACTATATATTTTGGAGAAATTCCACT |
| | CCTATGAATCTGAGTACTTACTCACTTTTTGATTTATCCAAGTTTCAATTAAAATTAAAAGGAACAC |
| | CAGTTCTTCAATTAAAGGAGAGTCAAATTAACGAACTCGTAATATCTCTCCTGTCGCAGGGTAAGTT |
| | ACTTATCCGTGACAATGATACACTCAGTGTTTCTACTGATGTTCTTGTTAACACCTACAGAAAGTTA |
| | CGTTGATGTAGGGCCAGATTCTGTTAAGTCTGCTTGTATTGAGGTTGATATACAACAGACTTTCTTT |
| | GATAAAACTTGGCCTAGGCCAATTGATGTTTCTAAGGCTGACGGTATTATATACCCTCAAGGCCGTA |
| | CATATTCTAACATAACTATCACTTATCAAGGTCTTTTTCCCTATCAGGGAGACCATGGTGATATGTA |
| | TGTTTACTCTGCAGGACATGCTACAGGCACAACTCCACAAAAGTTGTTTGTAGCTAACTATTCTCAG |
| | GACGTCAAACAGTTTGCTAATGGGTTTGTCGTCCGTATAGGAGCAGCTGCCAATTCCACTGGCACTG |
| | TTATTATTAGCCCATCTACCAGCGCTACTATACGAAAAATTTACCCTGCTTTTATGCTGGGTTCTTC |
| | AGTTGGTAATTTCTCAGATGGTAAAATGGGCCGCTTCTTCAATCATACTCTAGTTCTTTTGCCCGAT |
| | GGATGTGGCACTTTACTTAGAGCTTTTTATTGTATTCTAGAGCCTCGCTCTGGAAATCATTGTCCTG |
| | CTGGCAATTCCTATACTTCTTTTGCCACTTATCACACTCCTGCAACAGATTGTTCTGATGGCAATTA |
| | CAATCGTAATGCCAGTCTGAACTCTTTTAAGGAGTATTTTAATTTACGTAACTGCACCTTTATGTAC |
| | ACTTATAACATTACCGAAGATGAGATTTTAGAGTGGTTTGGCATTACACAAACTGCTCAAGGTGTTC |
| | ACCTCTTCTCATCTCGGTATGTTGATTTGTACGGCGGCAATATGTTTCAATTTGCCACCTTGCCTGT |
| | TTATGATACTATTAAGTATTATTCTATCATTCCTCACAGTATTCGTTCTATCCAAAGTGATAGAAAA |
| | GCTTGGGCTGCCTTCTACGTATATAAACTTCAACCGTTAACTTTCCTGTTGGATTTTTCTGTTGATG |
| | GTTATATACGCAGAGCTATAGACTGTGGTTTTAATGATTTGTCACAACTCCACTGCTCATATGAATC |
| | CTTCGATGTTGAATCTGGAGTTTATTCAGTTTCGTCTTTCGAAGCAAAACCTTCTGGCTCAGTTGTG |
| | GAACAGGCTGAAGGTGTTGAATGTGATTTTTCACCTCTTCTGTCTGGCACACCTCCTCAGGTTTATA |
| | ATTTCAAGCGTTTGGTTTTTACCAATTGCAATTATAATCTTACCAAATTGCTTTCACTTTTTTCTGT |
| | GAATGATTTTACTTGTAGTCAAATATCTCCAGCAGCAATTGCTAGTAACTGTTATTCTTCACTGATT |
| | TTGGATTATTTTTCATACCCACTTAGTATGAAATCCGATCTCAGTGTTAGTTCTGCTGGTCCAATAT |
| | CCCAGTTTAATTATAAACAGTCCTTTTCTAATCCCACATGTTTGATTTTAGCGACTGTTCCTCATAA |
| | CCTTACTACTATTACTAAGCCTCTTAAGTACAGCTATATTAACAAGTGCTCTCGTTTTCTTTCTGAT |
| | GATCGTACTGAAGTACCTCAGTTAGTGAACGCTAATCAATACTCACCCTGTGTATCCATTGTCCCAT |
| | CCACTGTGTGGGAAGACGGTGATTATTATAGGAAACAACTATCTCCACTTGAAGGTGGTGGCTGGCT |
| | TGTTGCTAGTGGCTCAACTGTTGCCATGACTGAGCAATTACAGATGGGCTTTGGTATTACAGTTCAA |
| | TATGGTACAGACACCAATAGTGTTTGCCCCAAGCTTGAATTTGCTAATGACACAAAAATTGCCTCTC |
| | AATTAGGCAATTGCGTGGAATATTCCCTCTATGGTGTTTCGGGCCGTGGTGTTTTTCAGAATTGCAC |
| | AGCTGTAGGTGTTCGACAGCAGCGCTTTGTTTATGATGCGTACCAGAATTTAGTTGGCTATTATTCT |
| | GATGATGGCAACTACTACTGTTTGCGTGCTTGTGTTAGTGTTCCTGTTTCTGTCATCTATGATAAAG |
| | AAACTAAAACCCACGCTACTCTATTTGGTAGTGTTGCATGTGAACACATTTCCTCTACCATGTCTCA |
| | ATACTCCCGTTCTACGCGATCAATGCTTAAACGGCGAGATTCTACATATGGCCCCCTTCAGACACCT |
| | GTTGGTTGTGTCCTAGGACTTGTTAATTCCTCTTTGTTCGTAGAGGACTGCAAGTTGCCTCTTGGTC |
| | AATCTCTCTGTGCTCTTCCTGACACACCTAGTACTCTCACACCTCGCAGTGTGCGCTCTGTTCCAGG |
| | TGAAATGCGCTTGGCATCCATTGCTTTTAATCATCCTATTCAGGTTGATCAACTTAATAGTAGTTAT |
| | TTTAAATTAAGTATACCCACTAATTTTTCCTTTGGTGTGACTCAGGAGTACATTCAGACAACCATTC |
| | AGAAAGTTACTGTTGATTGTAAACAGTACGTTTGCAATGGTTTCCAGAAGTGTGAGCAATTACTGCG |
| | CGAGTATGGCCAGTTTTGTTCCAAAATAAACCAGGCTCTCCATGGTGCCAATTTACGCCAGGATGAT |
| | TCTGTACGTAATTTGTTTGCGAGCGTGAAAAGCTCTCAATCATCTCCTATCATACCAGGTTTTGGAG |
| | GTGACTTTAATTTGACACTTCTAGAACCTGTTTCTATATCTACTGGCAGTCGTAGTGCACGTAGTGC |
| | TATTGAGGATTTGCTATTTGACAAAGTCACTATAGCTGATCCTGGTTATATGCAAGGTTACGATGAT |
| | TGCATGCAGCAAGGTCCAGCATCAGCTCGTGATCTTATTTGTGCTCAATATGTGGCTGGTTACAAAG |
| | TATTACCTCCTCTTATGGATGTTAATATGGAAGCCGCGTATACCTCATCTTTGCTTGGCAGCATAGC |
| | AGGTGTTGGCTGGACTGCTGGCTTATCCTCCTTTGCTGCTATTCCATTTGCACAGAGTATCTTTTAT |
| | AGGTTAAACGGTGTTGGCATTACTCAACAGGTTCTTTCAGAGAACCAAAAGCTTATTGCCAATAAGT |
| | TTAATCAGGCTCTGGGAGCTATGCAAACAGGCTTCACTACAACTAATGAAGCTTTTCACAAGGTTCA |
| | GGATGCTGTGAACAACAATGCACAGGCTCTATCCAAATTAGCTAGCGAGCTATCTAATACTTTTGGT |
| | GCTATTTCCGCCTCTATTGGAGACATCATACAACGTCTTGATGTTCTCGAACAGGACGCCCAAATAG |
| | ACAGACTTATTAATGGCCGTTTGACAACACTAAATGCTTTTGTTGCACAGCAGCTTGTTCGTTCCGA |
| | ATCAGCTGCTCTTTCGGCTCAATTGGCTAAAGATAAAGTCAATGAGTGTGTCAAGGCACAATCCAAG |
| | CGTTCTGGATTTTGCGGTCAAGGCACACATATAGTGTCCTTTGTTGTAAATGCCCCTAATGGCCTTT |
| | ACTTCATGCATGTTGGTTATTACCCTAGCAACCACATTGAGGTTGTTTCTGCTTATGGTCTTTGCGA |
| | TGCAGCTAACCCTACTAATTGTATAGCCCCTGTTAATGGCTACTTTATTAAAACTAATAACACTAGG |
| | ATTGTTGATGAGTGGTCATATACTGGCTCGTCCTTCTATGCACCTGAGCCCATCACCTCTCTTAATA |
| | CTAAGTATGTTGCACCACAGGTGACATACCAAAACATTTCTACTAACCTCCCTCCTCCTCTTCTCGG |
| | CAATTCCACCGGGATTGACTTCCAAGATGAGTTGGATGAGTTTTTCAAAAATGTTAGCACCAGTATA |
| | CCTAATTTTGGTTCTCTAACACAGATTAATACTACATTACTCGATCTTACCTACGAGATGTTGTCTC |
| | TTCAACAAGTTGTTAAAGCCCTTAATGAGTCTTACATAGACCTTAAAGAGCTTGGCAATTATACTTA |
| | TTACAACAAATGGCCGTGGTACATTTGGCTTGGTTTCATTGCTGGGCTTGTTGCCTTAGCTCTATGC |
| | GTCTTCTTCATACTGTGCTGCACTGGTTGTGGCACAAACTGTATGGGAAAACTTAAGTGTAATCGTT |
| | GTTGTGATAGATACGAGGAATACGACCTCGAGCCGCATAAGGTTCATGTTCACTAATTAACGAACTA |
| | TCAATGAGAGTTCAAAGACCACCCACTCTCTTGTTAGTGTTCTCACTCTCTCTTTTGGTCACTGCAT |
| | TTTCAAAACCTCTCTATGTACCTGAGCATTGTCAGAATTATTCTGGTTGCATGCTTAGGGCTTGTAT |
| | TAAAACTGCCCAAGCTGATACAGCTGGTCTTTATACAAATTTTCGAATTGACGTCCCATCTGCAGAA |
| | TCAACTGGTACTCAATCAGTTTCTGTCGATCGTGAGTCAACTTCAACTCATGATGGTCCTACCGAAC |
| | ATGTTACTAGTGTGAATCTTTTTGACGTTGGTTACTCAGTTAATTAACGAACTCTATGGATTACGTG |
| | TCTCTGCTTAATCAAATTTGGCAGAAGTACCTTAATTCACCGTATACTACTTGTTTGTATATCCCTA |
| | AACCCACAGCTAAGTATACACCTTTAGTTGGCACTTCATTGCACCCTGTGCTGTGGAACTGTCAGCT |
| | ATCCTTTGCTGGTTATACTGAATCTGCTGTTAATTCTACAAAAGCTTTGGCCAAACAGGACGCAGCT |
| | CAGCGAATCGCTTGGTTGCTACATAAGGATGGAGGAATCCCTGATGGATGTTCCCTCTACCTCCGGC |
| | ACTCAAGTTTATTCGCGCAAAGCGAGGAAGAGGAGTCATTCTCCAACTAAGAAACTGCGCTACGTTA |
| | AGCGTAGATTTTCTCTTCTGCGCCCTGAAGACCTTAGTGTTATTGTCCAACCAACACACTATGTCAG |
| | GGTTACATTTTCAGACCCCAACATGTGGTATCTACGTTCGGGTCATCATTTACACTCAGTTCACAAT |
| | TGGCTTAAACCTTATGGCGGCCAACCTGTTTCTGAGTACCATATTACTCTAGCTTTGCTAAATCTCA |
| | CTGATGAAGATTTAGCTAGAGATTTTTCACCCATTGCGCTCTTTTTGCGCAATGTCAGATTTGAGCT |
| | ACATGAGTTCGCCTTGCTGCGCAAAACTCTTGTTCTTAATGCATCAGAGATCTACTGTGCTAACATA |
| | CATAGATTTAAGCCTGTGTATAGAGTTAACACGGCAATCCCTACTATTAAGGATTGGCTTCTCGTTC |
| | AGGGATTTTCCCTTTACCATAGTGGCCTCCCTTTACATATGTCAATCTCTAAATTGCATGCACTGGA |
| | TGATGTTACTCGCAATTACATCATTACAATGCCATGCTTTAGAACTTATCCTCAACAAATGTTTGTT |
| | ACTCCTTTGGCCGTAGATGTTGTCTCCATACGGTCTTCCAATCAGGGTAATAAACAAATTGTTCATT |
| | CTTACCCCATTTTACATCATCCAGGATTTTAACGAACTATGGCTTTCTCGGCGTCTTTATTTAAACC |
| | CGTCCAGCTAGTCCCAGTTTCTCCTGCATTTCATCGCATTGAGTCTACTGACTCTATTGTTTTCACA |
| | TACATTCCTGCTAGCGGCTATGTAGCTGCTTTAGCTGTCAATGTGTGTCTCATTCCCCTATTATTAC |
| | TGCTACGTCAAGATACTTGTCGTCGCAGCATTATCAGAACTATGGTTCTCTATTTCCTTGTTCTGTA |
| | TAACTTTTTATTAGCCATTGTACTAGTCAATGGTGTACATTATCCAACTGGAAGTTGCCTGATAGCC |
| | TTCTTAGTTATCCTCATAATACTTTGGTTTGTAGATAGAATTCGTTTCTGTCTCATGCTGAATTCCT |
| | ACATTCCACTGTTTGACATGCGTTCTCACTTTATTCGTGTTAGTACAGTTTCTTCTCATGGTATGGT |
| | CCCTGTCATACACACCAAACCATTATTTATTAGAAACTTCGATCAGCGTTGCAGCTGTTCTCGTTGT |
| | TTTTATTTGCACTCTTCCACTTATATAGAGTGCACTTATATTAGCCGTTTTAGTAAGATTAGCCTAG |
| | TTTCTGTAACTGACTTCTCCTTAAACGGCAATGTTTCCACTGTTTTCGTGCCTGCAACGCGCGATTC |
| | AGTTCCTCTTCACATAATCGCCCCGAGCTCGCTTATCGTTTAAGCAGCTCTGCGCTACTATGGGTCC |
| | CGTGTAGAGGCTAATCCATTAGTCTCTCTTTGGACATATGGAAAACGAACTATGTTACCCTTTGTCC |
| | AAGAACGAATAGGGTTGTTCATAGTAAACTTTTTCATTTTTACCGTAGTATGTGCTATAACACTCTT |
| | GGTGTGTATGGCTTTCCTTACGGCTACTAGATTATGTGTGCAATGTATGACAGGCTTCAATACCCTG |
| | TTAGTTCAGCCCGCATTATACTTGTATAATACTGGACGTTCAGTCTATGTAAAATTCCAGGATAGTA |
| | AACCCCCTCTACCACCTGACGAGTGGGTTTAACGAACTCCTTCATAATGTCTAATATGACGCAACTC |
| | ACTGAGGCGCAGATTATTGCCATTATTAAAGACTGGAACTTTGCATGGTCCCTGATCTTTCTCTTAA |
| | TTACTATCGTACTACAGTATGGATACCCATCCCGTAGTATGACTGTCTATGTCTTTAAAATGTTTGT |
| | TTTATGGCTCCTATGGCCATCTTCCATGGCGCTATCAATATTTAGCGCCGTTTATCCAATTGATCTA |
| | GCTTCCCAGATAATCTCTGGCATTGTAGCAGCTGTTTCAGCTATGATGTGGATTTCCTACTTTGTGC |
| | AGAGTATCCGGCTGTTTATGAGAACTGGATCATGGTGGTCATTCAATCCTGAGACTAATTGCCTTTT |
| | GAACGTTCCATTTGGTGGTACAACTGTCGTACGTCCACTCGTAGAGGACTCTACCAGTGTAACTGCT |
| | GTTGTAACCAATGGCCACCTCAAAATGGCTGGCATGCATTTCGGTGCTTGTGACTACGACAGACTTC |
| | CTAATGAAGTCACCGTGGCCAAACCCAATGTGCTGATTGCTTTAAAAATGGTGAAGCGGCAAAGCTA |
| | CGGAACTAATTCCGGCGTTGCCATTTACCATAGATATAAGGCAGGTAATTACAGGAGTCCGCCTATT |
| | ACGGCGGATATTGAACTTGCATTGCTTCGAGCTTAGGCTCTTTAGTAAGAGTATCTTAATTGATTTT |
| | AACGAATCTCAATTTCATTGTTATGGCATCCCCTGCTGCACCTCGTGCTGTTTCCTTTGCCGATAAC |
| | AATGATATAACAAATACAAACCTGTCTCGAGGTAGAGGACGTAATCCAAAACCACGAGCTGCACCAA |
| | ATAACACTGTCTCTTGGTACACTGGGCTTACCCAACACGGGAAAGTCCCTCTTACCTTTCCACCTGG |
| | GCAGGGTGTACCTCTTAATGCCAATTCCACCCCTGCGCAAAATGCTGGGTATTGGCGGAGACAGGAC |
| | AGAAAAATTAATACCGGGAATGGAATTAAGCAACTGGCTCCCAGGTGGTACTTCTACTACACTGGAA |
| | CTGGACCCGAAGCAGCACTCCCATTCCGGGCTGTTAAGGATGGCATCGTTTGGGTCCATGAAGATGG |
| | CGCCACTGATGCTCCTTCAACTTTTGGGACGCGGAACCCTAACAATGATTCAGCTATTGTTACACAA |
| | TTCGCGCCCGGTACTAAGCTTCCTAAAAACTTCCACATTGAGGGGACTGGAGGCAATAGTCAATCAT |
| | CTTCAAGAGCCTCTAGCGTAAGCAGAAACTCTTCCAGATCTAGTTCACAAGGTTCAAGATCAGGAAA |
| | CTCTACCCGCGGCACTTCTCCAGGTCCATCTGGAATCGGAGCAGTAGGAGGTGATCTACTTTACCTT |
| | GATCTTCTGAACAGACTACAAGCCCTTGAGTCTGGCAAAGTAAAGCAATCGCAGCCAAAAGTAATCA |
| | CTAAGAAAGATGCTGCTGCTGCTAAAAATAAGATGCGCCACAAGCGCACTTCCACCAAAAGTTTCAA |
| | CATGGTGCAAGCTTTTGGTCTTCGCGGACCAGGAGACCTCCAGGGAAACTTTGGTGATCTTCAATTG |
| | AATAAACTCGGCACTGAGGACCCACGTTGGCCCCAAATTGCTGAGCTTGCTCCTACAGCCAGTGCTT |
| | TTATGGGTATGTCGCAATTTAAACTTACCCATCAGAACAATGATGATCATGGCAACCCTGTGTACTT |
| | CCTTCGGTACAGTGGAGCCATTAAACTTGACCCAAAGAATCCCAACTACAATAAGTGGTTGGAGCTT |
| | CTTGAGCAAAATATTGATGCCTACAAAACCTTCCCTAAGAAGGAAAAGAAACAAAAGGCACCAAAAG |
| | AAGAATCAACAGACCAAATGTCTGAACCTCCTAAGGAGCATCGTGTGCAAGGTACTCAGCGCACTCG |
| | CACCCGTCCAAGTGTTCAGCCTGGTCCAATGATTGATGTTAACACTGATTAGTGTCACTCAAAGTAA |
| | CAAGATCGCGGCAATCGTTTGTGTTTGGTAACCCCATCTCACCATCGCTTGTCCACTCTTGCACAGA |
| | ATGGAATCATGTTGTAATTACAGTGCAATAAGGTAATTATAACCCATTTAATTGATAGCTATGCTTT |
| | ATTAAAGTGTGTAGCTGTAGAGAGAATGTTAAAGACTGTCACCTCTGCGTGATTGCAAGTGAACAGT |
| | GCCCCCCGGGAAGAGCTCTACAGTGTGAAATGTAAATAAAAATAGCTATTATTCAATTAGATTAGGC |
| | TAATTAGATGATTTGCAAAAAAAAAAAA |
| 4 | GenBank Accession No. KX154693.1 (Middle East Respiratory Syndrome |
| | Coronavirus Isolate Hu/Riyadh-KSA-13127/2016, complete genome) |
| | ATCTCACTTCCCCTCGTTCTCTTGCAGAACTTTGATTTTAACGAACTTAAATAAAAGCCCTGTTGTT |
| | TAGCGTATTGTTGCACTTGTCTGGTGGGATTGTGGCACTAATCTGCCTGCTCATCTAGGCAGTGGAC |
| | ATATGCTCAACACTGGGTATAATTCTAATTGAATACTATTTTTCAGTTAGAGCGTCGTGTCTCTTGT |
| | ACGTCTCGGTCACAATATACGGTTTCGTCCGGTGCGTGGCAATTCGGGGCACATCATGTCTTTCGTG |
| | GCTGGTGTGATCGCGCAAGGTGCGCGCGGTACGTATCGAGCAGCGCTCAACTCTGAAAAACATCAAG |
| | ACCATGTGTCTCTAACTGTGCCACTCTGTGGTTCAGGAAACCTGGTTGAAAAACTTTCACCATGGTT |
| | CATGGATGGCGAAAATGCCTATGAAGTGGTGAAGGCCATGTTACTTAAAAAAGAGCCACTTCTCTAT |
| | GTGCCCATCCGGCTGGCTGGACACACTAGACACCTCCCAGGTCCTCGTGTGTACCTGGTTGAGAGGC |
| | TCATTGCTTGTGAAAATCCATTCATGGTTAACCAATTGGCTTATAGCTCTAGTGCAAATGGCAGCCT |
| | GGTTGGCACAACTTTGCAGGGCAAGCCTATTGGTATGTTCTTCCCTTATGACATCGAACTTGTCACA |
| | GGAAAGCAAAATATTCTCCTGCGCAAGTATGGCCGTGGTGGTTATCACTACACCCCAGTCCACTATG |
| | AGCGAGACAACACCTCTTGCCCTGAGTGGATGGACGATTTTGAGGCGGATCCTAAAGGCAAATATGC |
| | CCAGAATCTGCTTAAGAAGTTGATTGGCGGTGATGTCACTCCAGTTGACCAATACATGTGTGGCGTT |
| | GATGGAAAACCCATTAGTGCCTACGCATTTTTAATGGCCAAGGATGGAATAACCAAACTGGCTGATG |
| | TTGAAGCGGACGTCGCAGCACGTGCTGATGACGAAGGCTTCATCACATTAAAGAACAATCTATATAG |
| | ATTGGTTTGGCATGTTGAGCGTAAAGACGTTCCATATCCTAAGCAATCTATTTTTACTATTAATAGT |
| | GTGGTCCAAAAGGATGGTGTTGAAAACACTCCTCCTCACTATTTTACTCTTGGATGCAAAATTTTAA |
| | CGCTCACCCCACGTAACAAGTGGAGTGGCGTTTCTGACTTGTCCCTCAAACAAAAACTCCTTTACAC |
| | CTTCTATGGTAAGGAGTCACTTGAGAACCCAACCTACATTTACCACTCCGCATTCATTGAGTGTGGA |
| | AGTTGTGGTAATGATTCCTGGCTTACAGGGAATGCTATCCAAGGGTTTGCCTGTGGATGTGGGGCAT |
| | CATATACAGCTAATGATGTTGAAGTCCAATCATCTGGCATGATTAAGCCAAATGCTCTTCTTTGTGC |
| | TACTTGCCCCTTTGCTAAGGGTGACAGCTGTTCTTCTAATTGCAAACATTCAGTTGCTCAGTTGGTT |
| | AGTTACCTTTCTGAACGCTGTAATGTTATTGCTGATTCTAAGTCCTTCACACTTATCTTTGGTGGCG |
| | TAGCTTACGCCTACTTTGGATGTGAGGAAGGTACTATGTACTTTGTGCCTAGAGCTAAGTCTGTTGT |
| | CTCAAGGATTGGAGACTCCATCTTTACAGGCTGTACTGGCTCTTGGAACAAGGTCACTCAAATTGCT |
| | AACATGTTCTTGGAACAGACTCAGCATTCCCTTAACTTTGTGGGAGAGTTCGTTGTCAACGATGTTG |
| | TCCTCGCAATTCTCTCTGGAACCACAACTAATGTTGACAAAATACGCCAGCTTCTCAAAGGTGTCAC |
| | CATTGACAAGTTGCGTGATTATTTAGCTGACTATGACGTAGCAGTCACTGCCGGCCCATTCATGGAT |
| | AATGCTATTAATGTTGGTGGTACAGGATTACAGTATGCCGCCATTACTGCACCTTATGTAGTTCTCA |
| | CTGGCTTAGGTGAGTCCTTTAAGAAAGTTGCAACCATACCGTACAAGGTTTGCAACTCTGTTAAGGA |
| | TACTCTGACTTATTATGCTCACAGCGTGTTGTACAGAGTTTTTCCTTATGACATGGATTCTGGTGTG |
| | TCATCCTTTAGTGAACTACTTTTTGATTGCGTTGATCTTTCAGTAGCTTCTACCTATTTTTTAGTCC |
| | GCCTCTTGCAAGATAAGACTGGCGACTTTATGTCTACAATTATTACTTCCTGCCAAACTGCTGTTAG |
| | TAAGCTTCTAGATACATGTTTTGAAGCTACAGAAGCAACATTTAACTTCTTGTTAGATTTGGCAGGA |
| | TTGTTCAGAATCTTTCTTCGCAATGCCTATGTGTACACTTCACAAGGGTTTGTGGTGGTCAATGGCA |
| | AAGTTTCTACACTTGTCAAACAAGTGTTAGACTTGCTTAATAAGGGTATGCAACTTTTGCATACAAA |
| | GGTCTCCTGGGCTGGTTCTAACATCAGTGCTGTTATCTACAGCGGCAGGGAGTCTCTAATATTCCCA |
| | TCGGGAACCTATTACTGTGTCACCACTAAGGCTAAGTCCGTTCAACAAGATCTTGACGTTATTTTGC |
| | CTGGTGAGTTTTCCAAGAAGCAGTTAGGACTGCTCCAACCTACTGACAATTCTACAACTGTTAGTGT |
| | TACTGTATCCAGTAACATGGTTGAAACTGTTGTGGGTCAACTTGAGCAAACTAATATGCACAGTCCT |
| | GATGTTATAGTAGGTGACTATGTCATTATTAGTGAAAAATTGTTTGTGCGTAGTAAGGAAGAAGACG |
| | GATTCGCCTTCTACCCTGCTTGCACTAATGGTCATGCTGTACCGACTCTCTTTAGACTTAAGGGAGG |
| | TGCACCTGTAAAAAAAGTAGCCTTTGGCGGTGATCAAGTACATGAGGTTGCTGCTGTAAGAAGTGTT |
| | ACTGTCGAGTACAACATTCATGCTGTATTAGACACACTACTTGCTTCTTCTAGTCTTAGAACCTTTG |
| | TTGTAGATAAGTCTTTGTCAATTGAGGAGTTTGCTGACGTAGTAAAGGAACAAGTCTCAGACTTGCT |
| | TGTTAAATTACTGCGTGGAATGCCGATTCCAGATTTTGATTTAGACGATTTTATTGACGCACCATGC |
| | TATTGCTTTAACGCTGAGGGTGATGCATCTTGGTCTTCTACTATGATCTTCTCTCTTCACCCCGTCG |
| | AGTGTGACGAGGAGTGTTCTGAAGTAGAGGCTTCAGATTTAGAAGAAGGTGAATCAGAGTGCATTTC |
| | TGAGACTTCAACTGAACAAGTTGACGTTTCTCATGAGGTTTCTGACGACGAGTGGGCTGCTGCAGTT |
| | GATGAAGCGTTCCCCCTCGATGAAGCAGAAGATGTTACTGAATCTGTGCAAGAAGAAGCACAACCAG |
| | TAGAAGTACCTGTTGAAGATATTGTGCAGGTTGTCATAGTTGACACCTTACAGGAAACTCCTGTTGT |
| | GTCTGATACTGTTGAAGTCCCACCGCAAGTGGTGAAACTTCCGTCTGAACCTCAGACTATCCAGCCC |
| | GAGGTAAAAGAAGTTGCACCTGTCTATGAGGCTGATACCGAACAGACACAGAGTGTTACTGTTAAAC |
| | CTAAGAGGTTACGCAAAAAGCGTAATGTTGACCCTTTGTCCAATTTTGAACATAAGGTTATTACAGA |
| | GTGCGTTACCATAGTTTTAGGTGACGCAATTCAAGTAGCCAAGTGCTATGGGGAGTCTGTGTTAGTT |
| | AATGCTGCTAACACACATCTTAAGCATGGCGGTGGTATCGCTGGTGCTATTAATGCGGCTTCAAAAG |
| | GGGCTGTCCAAAAAGAGTCAGATGAGTATATTCTGGCTAAAGGGCCGTTACAAGTAGGAGATTCAGT |
| | TCTCTTGCAAGGCCATTCTCTAGCTAAGAATATCCTGCATATCGTAGGCCCAGATGCCCGCGCTAAA |
| | CAGGATGTTTCTCTCCTTAGTAAGTGCTATAAGGCTATGAATGCATATCCTCTTGTAGTCACTCCTC |
| | TTGTTTCAACAGGCATATTTGGTGTAAAACCAGCTGTGTCTTTTGATTATCTTATTAGAGAGGCTAA |
| | GACTAGAGTTTTAGTCGTCGTTAATTCCCAAGATGTCTATAAGAGTCTTACCATAGTTGACATTCCA |
| | CAGAGTTTGACTTTTTCATATGATGGGTTACGTGGCGCAATACGTAAAGCTAAAGATTATGGTTTTA |
| | CTGTTTTTGTGTGCACAGACAACTCTGCTAACACTAAAGTTCTTAGGAACAAGGGTGTTGATTATAC |
| | TAAGAAGTTTCTTACAGTTGACGGTGTGCAATATTATTGCTACACGTCTAAGGACACTTTAGATGAT |
| | ATCTTACAACAGGCTAATAAGTCTGTTGGTATTATATCTATGCCTTTGGGATATGTGTCTCATGGTT |
| | TAGACTTAATTCAAGCAGGGAGTGTCGTGCGTAGAGTTAACGTGCCCTACGTGTGTCTCCTAGCTAA |
| | TAAAGAGCAAGAAGCTATTTTGATGTCTGAAGACGTTAAGTTAAACCCTTCAGAAGATTTTATAAAG |
| | CACGTCCGCACTAATGGTGGTTACAATTCTTGGCATTTAGTCGAGGGTGAACTATTGGTGCAAGACT |
| | TACGCTTAAATAAGCTCCTGCATTGGTCTGATCAAACCATATGCTACAAGGATAGTGTGTTTTATGT |
| | TGTAAAGAATAGTACAGCTTTTCCATTTGAAACACTTTCAGCATGTCGTGCGTATTTGGATTCACGC |
| | ACGACACAGCAGTTAACAATCGAAGTCTTAGTGACTGTCGATGGTGTAAATTTTAGAACAGTCGTTC |
| | TAAATAATAAGAACACTTATAGATCACAGCTTGGATGCGTTTTCTTTAATGGTGCTGATATTTCTGA |
| | TACCATTCCTGATGAGAAACAGAATGGTCACAGTTTATATCTAGCAGACAATTTGACTGCTGATGAA |
| | ACAAAGGCGCTTAAAGAGTTATATGGCCCCGTTGATCCTACTTTCTTACACAGATTCTATTCACTTA |
| | AGGCTGCAGTCCATAAGTGGAAGATGGTTGTGTGTGATAAGGTACGTTCTCTCAAATTGAGTGATAA |
| | TAATTGTTATCTTAATGCAGTTATTATGACACTTGATTTATTGAAGGACATTAAATTTGTTATACCT |
| | GCTCTACAGCATGCATTTATGAAACATAAGGGCGGTGATTCAACTGACTTCATAGCCCTCATTATGG |
| | CTTATGGCAATTGCACATTTGGTGCTCCAGATGATGCCTCTCGGTTACTTCATACCGTGCTTGCAAA |
| | GGCTGAGTTATGCTGTTCTGCACGCATGGTTTGGAGAGAGTGGTGCAATGTCTGTGGCATAAAAGAT |
| | GTTGTTCTACAAGGCTTAAAAGCTTGTTGTTACGTGGGTGTGCAAACTGTTGAAGATCTGCGTGCTC |
| | GCATGACATATGTATGCCAGTGTGGTGGTGAACGTCATCGGCAAATAGTCGAACACACCACCCCCTG |
| | GTTGCTGCTCTCAGGCACACCAAATGAAAAATTGGTGACAACCTCCACGGCGCCTGATTTTGTAGCG |
| | TTTAATGTCTTTCAGGGCATTGAAACGGCTGTTGGCCATTATGTTCATGCTCGCCTGAAGGGTGGTC |
| | TTATTTTAAAGTTTGACTCTGGCACCGTTAGCAAGACTTCAGACTGGAAGTGCAAGGTGACAGATGT |
| | ACTTTTCCCCGGCCAAAAATACAGTAGCGATTGTAATGTCGTACGGTATTCTTTGGACGGTAATTTC |
| | AGAACAGAGGTTGATCCCGACCTATCTGCTTTCTATGTTAAGGATGGTAAATACTTTACAAGTGCAC |
| | CACCCGTAACATATTCACCAGCTACAATTTTAGCTGGTAGTGTCTACACTAATAGCTGCCTTGTATC |
| | GTCTGATGGACAACCTGGCGGTGATGCTATTAGTTTGAGTTTTAATAACCTTTTAGGGTTTGATTCT |
| | AGTAAACCAGTCACTAAGAAATACACTTACTCCTTCTTGCCTAAAGAAGACGGCGATGTGTTGTTGG |
| | CTGAGTTTGACACTTATGACCCTATTTATAAGAATGGTGCCATGTATAAAGGCAAACCAATTCTTTG |
| | GGTCAACAAAGCATCTTATGATACTAATCTTAATAAGTTCAATAGAGCTAGTTTGCGTCAAATTTTT |
| | GACGTAGCCCCCATTGAACTCGAAAATAAATTCACACCTTTGAGTGTGGCGTCCACACCAGTTGAAC |
| | CTCCAACTGTAGATGTGGTAGCACTTCAACAGGAAATGACAATTGTCAAATGTAAGGGTTTAAATAA |
| | ACCTTTCGTGAAGGACAATGTCAGTTTCGTTGTTGATGACTCAGGTACTCCCGTTGTTGAGTATCTG |
| | TCTAAAGAAGATCTACATACATTGTATGTAGACCCTAAGTATCAAGTCATTGTCTTAAAAGACAATG |
| | TACTTTCTTCTATGCTTAGATTGCACACCGTTGAGTCAGGTGATATTAACGTTGTTGCAGCTTCCGG |
| | ATCTTTGACACGTAAAGTGAAGTTACTATTTAGGGCTTCATTTTATTTCAAAGAATTTGCTACCCGC |
| | ACTTTCACTGCTACCACTGCTGTAGGTAGTTGTATAAAGAGTGTAGTGCGGCATCTAGGTGTTACTA |
| | AAGGCATATTGACAGGCTGTTTTAGTTTTGTCAAGATGTTATTTATTCTTCCACTAGCTTACTTTAG |
| | TGATTCAAAACTCGGCACCACAGAGGTTAAAGTGAGTGCTTTGAAAACAGCTGGCGTTGTGACAGGT |
| | AATGTTGTAAAACAGTGTTGCACTGCTGCTGTTGATTTAAGTATGGATAAGTTGCGCCGTGTGGATT |
| | GGAAATCAACCCTACGGTTGTTACTTATGTTATGCACAACTATGGTATTGTTGTCTTCTGTGTATCA |
| | CTTGTATGTCTTCAATCAGGTCTTATCAAGTGATGTTATGTTTGAAGATGCCCAAGGTTTGAAAAAG |
| | TTCTACAAAGAAGTTAGAGCTTACCTAGGAATCTCTTCTGCTTGTGACGGTCTTGCTTCAGCTTATA |
| | GGGCGAATTCATTTGATGTACCTACATTCTGCGCAAACCGTTCTGCAATGTGTAATTGGTGCTTGAT |
| | TAGCCAAGATTCCATAACTCACTACCCAGCTCTTAAGATGGTTCAAACACATCTTAGCCACTATGTT |
| | CTTAACATAGATTGGTTGTGGTTTGCATTTGAGACTGGTTTGGCATACATGCTCTATACCTCGGCCT |
| | TCAACTGGTTGTTGTTGGCAGGTACATTGCATTATTTCTTTGCACAGACTTCCATATTTGTAGACTG |
| | GCGGTCATACAATTATGCTGTGTCTAGTGCCTTCTGGTTATTCACCCACATTCCAATGGCGGGTTTG |
| | GTACGAATGTATAATTTGTTAGCATGCCTTTGGCTTTTACGCAAGTTTTATCAGCATGTAATCAATG |
| | GTTGCAAAGATACGGCATGCTTGCTCTGCTATAAGAGGAACCGACTTACTAGAGTTGAAGCTTCTAC |
| | CGTTGTCTGTGGTGGAAAACGTACGTTTTATATCACAGCAAATGGCGGTATTTCATTCTGTCGTAGG |
| | CATAATTGGAATTGTGTGGATTGTGACATTGCAGGTGTGGGGAATACCTTCATCTGTGAAGAAGTCG |
| | CAAATGACCTCACTACCGCCCTACGCAGGCCTATTAACGCTACGGATAGATCACATTATTATGTGGA |
| | TTCCGTTACAGTTAAAGAGACTGTTGTTCAGTTTAATTATCGTAGAGACGGTCAACCATTCTACGAG |
| | CGGTTTCCCCTCTGCGCTTTTACAAATCTAGATAAGTTGAAGTTCAAAGAGGTCTGTAAAACTACTA |
| | CTGGTATACCTGAATACAACTTTATCATCTACGACTCATCAGATCGTGGCCAGGAAAGTTTAGCTAG |
| | GTCTGCATGTGTTTATTATTCTCAAGTCTTGTGTAAATCAATTCTTTTGGTTGACTCAAGTTTGGTT |
| | ACTTCTGTTGGTGATTCTAGTGAAATCGCCACTAAAATGTTTGATTCCTTTGTTAATAGTTTCGTCT |
| | CGCTGTATAATGTCACACGCGATAAGTTGGAAAAACTTATCTCTACTGCTCGTGATGGCGTAAGGCG |
| | AGGCGATAACTTCCATAGTGTCTTAACAACATTCATTGACGCAGCACGAGGCCCCGCAGGTGTGGAG |
| | TCTGATGTTGAGACCAATGAAATTGTTGACTCTGTGCAGTATGCTCATAAACATGACATACAAATTA |
| | CTAATGAGAGTTACAATAATTATGTACCCTCATATGTTAAACCTGATAGTGTGTCTACCAGTGATTT |
| | AGGTAGTCTCATTGATTGTAATGCGGCTTCAGTTAACCAAATTGTCTTGCGTAATTCTAATGGTGCT |
| | TGTATTTGGAACGCTGCTGCATATATGAAACTCTCGGATGCACTTAAACGACAGATTCGCATTGCAT |
| | GCCGTAAGTGTAATTTAGCTTTCCGGTTAACCACCTCAAAGCTACGCGCTAATGATAATATCTTATC |
| | AGTTAGATTCACTGCTAACAAAATTGTTGGTGGTGCTCCTACATGGTTTAATGTGTTGCGTGACTTT |
| | ACGTTAAAGGGTTACGTTCTTGCTACCATTATTGTGTTTCTGTGTGCTGTACTGATGTATTTGTGTT |
| | TACCTACATTTTCTATGGTACCTGTTGAATTTTATGAAGACCGCATCTTGGACTTTAAAGTTCTTGA |
| | TAATGGTATCATTAGGGATGTAAATCCTGATGATAAGTGCTTTGCTAATAAGCACCGGTCCTTCACA |
| | CAATGGTATCATGAGCATGTTGGTGGTGTCTATGACAACTCTATCACATGCCCATTGACAGTTGCAG |
| | TAATTGCTGGAGTTGCTGGTGCTCGCATTCCAGACGTACCTACTACATTGGCTTGGGTGAACAATCA |
| | GATAATTTTCTTTGTTTCTCGAGTCTTTGCTAATACAGGCAGTGTTTGCTACACTCCTATAGATGAG |
| | ATACCCTATAAGAGTTTCTCTGATAGTGGTTGCATTCTTCCATCTGAGTGCACTATGTTTAGGGATG |
| | CAGAGGGCCGTATGACACCATACTGCCATGATCCTACTGTTTTGCCTGGGGCTTTTGCGTACAGTCA |
| | GATGAGGCCTCATGTTCGTTACGACTTGTATGATGGTAACATGTTTATTAAATTTCCTGAAGTAGTA |
| | TTTGAAAGTACACTTAGGATTACTAGAACTCTGTCAACTCAGTACTGCCGGTTCGGTAGTTGTGAGT |
| | ATGCACAAGAGGGTGTTTGTATTACCACAAATGGCTCGTGGGCCATTTTTAATGACCACCATCTTAA |
| | TAGACCTGGTGTCTATTGTGGCTCTGATTTTATTGACATTGTCAGGCGGTTAGCAGTATCACTGTTC |
| | CAGCCTATTACTTATTTCCAATTGACTACCTCATTGGTCTTGGGTATAGGTTTGTGTGCATTCCTGA |
| | CTTTGCTCTTCTATTATATTAATAAAGTAAAACGTGCTTTTGCAGATTACACCCAGTGTGCTGTAAT |
| | TGCTGTTGTTGCTGCTGTTCTTAATAGCTTGTGCATCTGCTTTGTTGCCTCTATACCATTGTGTATA |
| | GTACCTTACACTGCATTGTACTATTATGCTACATTCTATTTTACTAATGAGCCTGCATTTATTATGC |
| | ATGTTTCTTGGTACATTATGTTCGGGCCTATCGTTCCCATATGGATGACCTGCGTCTATACAGTTGC |
| | AATGTGCTTTAGACACTTCTTCTGGGTTTTAGCTTATTTTAGTAAGAAACATGTAGAAGTTTTTACT |
| | GACGGTAAGCTTAATTGTAGTTTCCAGGACGCTGCCTCTAATATCTTTGTTATTAACAAGGACACTT |
| | ATGCAGCTCTTAGAAACTCTTTAACTAATGATGCCTATTCACGATTTTTGGGGTTGTTTAACAAGTA |
| | TAAGTACTTCTCTGGTGCTATGGAAACAGCCGCTTATCGTGAAGCTGCAGCATGTCATCTTGCTAAA |
| | GCCTTACAAACATACAGCGAGACTGGTAGTGATCTTCTTTACCAACCACCCAACTGTAGCATAACCT |
| | CTGGCGTGTTGCAAAGCGGTTTGGTGAAAATGTCACATCCCAGTGGAGATGTTGAGGCTTGTATGGT |
| | TCAGGTTACCTGCGGTAGCATGACTCTTAATGGTCTTTGGCTTGACAACACAGTCTGGTGCCCACGA |
| | CACGTAATGTGCCCGGCTGACCAGTTGTCTGATCCTAATTATGATGCCTTGTTGATTTCTATGACTA |
| | ATCATAGTTTCAGTGTGCAAAAACACATTGGCGCTCCAGCAAACTTGCGTGTTGTTGGTCATGCCAT |
| | GCAAGGCACTCTTTTGAAGTTGACTGTCGATGTTGCTAACCCTAGCACTCCAGCCTACACTTTTACA |
| | ACAGTGAAACCTGGCGCAGCATTTAGTGTGTTAGCATGCTATAATGGTCGTCCGACTGGTACATTCA |
| | CTGTTGTAATGCGCCCTAACTACACAATTAAGGGTTCCTTTCTGTGTGGTTCTTGTGGTAGTGTTGG |
| | TTACACCAAGGAGGGTAGTGTGATCAATTTTTGTTACATGCATCAAATGGAACTTGCTAATGGTACA |
| | CATACCGGTTCAGCATTTGATGGTACTATGTATGGTGCCTTTATGGATAAACAAGTGCACCAAGTTC |
| | AGTTAACAGACAAATACTGCAGTGTTAATGTAGTAGCTTGGCTTTACGCAGCAATACTTAATGGTTG |
| | CGCTTGGTTTGTAAAACCTAATCGCACTAGTGTTGTTTCTTTTAATGAATGGGCTCTTGCCAACCAA |
| | TTCACTGAATTTGTTGGCACTCAATCCGTTGACATGTTAGCTGTCAAAACAGGCGTTGCTATTGAAC |
| | AGCTGCTTTATGCGATCCAACAACTTTATACTGGGTTCCAGGGAAAGCAAATCCTTGGCAGTACTAT |
| | GTTGGAAGATGAATTCACACCTGAGGATGTTAATATGCAGATTATGGGTGTGGTTATGCAGAGTGGT |
| | GTGAGAAAAGTTACATATGGTACTGCGCATTGGTTGTTCGCGACCCTTGTCTCAACCTATGTGATAA |
| | TCTTACAAGCCACTAAATTTACTTTGTGGAACTACTTGTTTGAGACTATTCCCACACAGTTGTTCCC |
| | ACTCTTATTTGTGACTATGGCCTTCGTTATGTTGTTGGTTAAACACAAACACACCTTTTTGACACTT |
| | TTCTTGTTGCCTGTGGCTATTTGTTTGACTTATGCAAACATAGTCTACGAGCCCACTACTCCCATTT |
| | CGTCAGCGCTGATTGCAGTTGCAAATTGGCTTGCCCCCACTAATGCTTATATGCGCACTACACATAC |
| | TGATATTGGTGTCTACATTAGTATGTCACTTGTATTAGTCATTGTAGTGAAGAGATTGTACAACCCA |
| | TCACTTTCTAACTTTGCGTTAGCATTGTGCAGTGGTGTAATGTGGTTGTACACTTATAGCATTGGAG |
| | AAGCCTCAAGCCCCATTGCCTATCTGGTTTTTGTCACTACACTCACTAGTGATTATACGATTACAGT |
| | CTTTGTTACTGTCAACCTTGCAAAAGTTTGCACTTATGCCATCTTTGCTTACTCACCACAGCTTACA |
| | CTTGTGTTTCCGGAAGTGAAGATGATACTTTTATTATACACATGTTTAGGTTTCATGTGTACTTGCT |
| | ATTTTGGTGTCTTCTCTTTTTTGAACCTTAAGCTTAGAGCACCTATGGGTGTCTATGACTTTAAGGT |
| | CTCAACACAAGAGTTCAGATTCATGACTGCTAACAATCTAACTGCACCTAGAAATTCTTGGGAGGCT |
| | ATGGCTCTGAACTTTAAGTTAATAGGTATTGGCGGTACACCTTGTATAAAGGTTGCTGCTATGCAGT |
| | CTAAACTTACAGATCTTAAATGCACATCTGTGGTTCTCCTCTCTGTGCTCCAACAGTTACACTTAGA |
| | GGCTAATAGTAGGGCCTGGGCTTTCTGTGTTAAATGCCATAATGATATATTGGCAGCAACAGACCCC |
| | AGTGAGGCTTTCGAGAAATTCGTAAGTCTCTTTGCCACTTTAATGACTTTTTCTGGTAATGTAGATC |
| | TTGATGCGTTAGCTAGTGATATTTTTGACACTCCTAGCGTACTTCAAGCTACTCTTTCTGAGTTTTC |
| | ACACTTAGCTACCTTTGCTGAGTTGGAAGCTGCGCAGAAAGCCTATCAGGAAGCTATGGACTCTGGT |
| | GACACCTCACCACAAGTTCTTAAGGCTTTGCAGAAGGCTGTTAATATAGCTAAAAACGCCTATGAGA |
| | AGGATAAGGCAGTGGCCCGTAAGTTAGAACGTATGGCTGATCAGGCTATGACTTCTATGTATAAGCA |
| | AGCACGTGCTGAAGACAAGAAAGCAAAAATTGTCAGTGCTATGCAAACTATGTTGTTTGGTATGATT |
| | AAGAAGCTCGACAACGATGTTCTTAATGGTATCATTTCTAACGCTAGGAATGGTTGTATACCTCTTA |
| | GTGTCATTCCACTGTGTGCTTCAAATAAACTTCGCGTTGTAATTCCTGACTTCACCGTCTGGAATCA |
| | GGTAGTCACATATCCCTCGCTTAACTACGCTGGGGCTTTGTGGGACATTACAGTTATAAACAATGTG |
| | GACAATGAAATTGTTAAGTCTTCAGATGTTGTAGACAGCAATGAAAATTTAACATGGCCACTTGTTT |
| | TAGAATGCACTAGGGCATCCACTTCTGCCGTTAAGTTGCAAAATAATGAGATCAAACCTTCAGGTTT |
| | AAAAACCATGGTTGTGTCTGCAGGTCAAGAGCAAACTAACTGTAATACTAGTTCCTTAGCTTATTAC |
| | GAACCTGTGCAGGGTCGTAAAATGCTGATGGCTCTTCTTTCTGATAATGCCTATCTCAAATGGGCGC |
| | GTGTTGAAGGTAAGGACGGATTTGTTAGTGTAGAGCTACAACCTCCTTGCAAATTCTTGATTGCGGG |
| | ACCAAAAGGACCTGAAATCCGATATCTCTATTTTGTTAAAAATCTTAACAACCTTCATCGCGGGCAA |
| | GTGTTAGGGCACATTGCTGCGACTGTTAGATTGCAAGCTGGTTCTAACACCGAGTTTGCCTCTAATT |
| | CTTCGGTGTTGTCACTTGTTAACTTCACCGTTGATCCTCAAAAAGCTTATCTCGATTTCGTCAATGC |
| | GGGAGGTGCCCCATTGACAAATTGTGTTAAGATGCTTACTCCTAAAACTGGTACAGGTATAGCTATA |
| | TCTGTTAAACCAGAGAGTACAGCTGATCAAGAGACTTATGGTGGAGCTTCAGTGTGTCTCTATTGCC |
| | GTGCGCATATAGAACATCCTGATGTCTCTGGTGTTTGTAAATATAAGGGTAAGTTTGTCCAAATCCC |
| | TGCTCAGTGTGTCCGTGACCCTGTGGGATTTTGTTTGTCAAATACCCCCTGTAATGTCTGTCAATAT |
| | TGGATTGGATATGGGTGCAATTGTGACTCGCTTAGGCAAGTAGCACTGCCCCAATCTAAAGATTCCA |
| | ATTTTTTAAACGAGTCCGGGGTTCTATTGTAAATGCCCGAATAGAACCCTGTTCAAGTGGTTTGTCC |
| | ACTGATGTCGTCTTTAGGGCATTTGACATCTGCAACTATAAGGCTAAGGTTGCTGGTATTGGAAAAT |
| | ACTACAAGACTAATACTTGTAGGTTTGTAGAATTAGATGACCAAGGGCATCATTTAGACTCCTATTT |
| | TGTCGTTAAGAGGCATACTATGGAGAATTATGAACTAGAGAAGCACTGTTACGATTTGTTACGTGAC |
| | TGTGATGCTGTAGCTCCCCATGATTTCTTCATCTTTGATGTAGACAAAGTTAAAACACCTCATATTG |
| | TACGTCAGCGTTTAACTGAGTACACTATGATGGATCTTGTATATGCCCTGAGGCACTTTGATCAAAA |
| | TAGCGAAGTGCTTAAGGCTATCTTAGTGAAGTATGGTTGCTGTGATGTTACCTACTTTGAAAATAAA |
| | CTCTGGTTTGATTTTGTTGAAAATCCCAGTGTTATTGGTGTTTATCATAAACTTGGAGAACGTGTAC |
| | GCCAAGCTATCTTAAACACTGTTAAATTTTGTGACCACATGGTCAAGGCTGGTTTAGTCGGTGTGCT |
| | CACACTAGACAACCAGGACCTTAATGGCAAGTGGTATGATTTTGGTGACTTCGTAATCACTCAACCT |
| | GGTTCAGGAGTAGCTATAGTTGATAGCTACTATTCTTATTTGATGCCTGTGCTCTCAATGACCGATT |
| | GTTTGGCCGCTGAGACACATAGGGATTGTGATTTTAATAAACCACTCATTGAGTGGCCACTTACTGA |
| | GTATGATTTTACTGATTATAAGGTACAACTCTTTGAGAAGTACTTTAAATATTGGGATCAGACGTAT |
| | CACGCAAATTGCGTTAATTGTACTGATGACCGTTGTGTGTTACATTGTGCTAATTTCAATGTATTGT |
| | TTGCTATGACCATGCCTAAGACTTGTTTCGGACCCATAGTCCGAAAGATCTTTGTTGATGGCGTGCC |
| | ATTTGTAGTATCTTGTGGTTATCACTACAAAGAATTAGGTTTAGTCATGAATATGGATGTTAGTCTC |
| | CATAGACATAGGCTCTCTCTTAAGGAGTTGATGATGTATGCCGCTGATCCAGCCATGCACATTGCCT |
| | CCTCTAACGCTTTTCTTGATTTGAGGACATCATGTTTTAGTGTCGCTGCACTTACAACTGGTTTGAC |
| | TTTTCAAACTGTGCGGCCTGGCAATTTTAACCAAGACTTCTATGATTTCGTGGTATCTAAAGGTTTC |
| | TTTAAGGAGGGCTCTTCAGTTACGCTCAAACATTTTTTCTTTGCTCAAGATGGTAATGCTGCTATTA |
| | CAGATTATAATTACTATTCTTATAATCTGCCTACTATGTGTGACATCAAACAAATGTTGTTCTGCAT |
| | GGAAGTTGTAAACAAGTACTTCGAAATCTATGACGGTGGTTGTCTTAATGCTTCTGAAGTGGTTGTT |
| | AATAATTTAGACAAGAGTGCTGGCCATCCTTTTAATAAGTTTGGCAAAGCTCGTGTCTATTATGAGA |
| | GCATGTCTTACCAGGAGCAAGATGAACTCTTTGCCATGACAAAGCGTAACGTCATTCCTACCATGAC |
| | TCAAATGAATCTAAAATATGCTATTAGTGCTAAGAATAGAGCTCGCACTGTTGCAGGCGTGTCCATA |
| | CTTAGCACAATGACTAATCGCCAGTACCATCAGAAAATGCTTAAGTCCATGGCTGCAACTCGTGGAG |
| | CGACTTGCGTCATTGGTACTACAAAGTTCTATGGTGGCTGGGATTTCATGCTTAAAACATTGTACAA |
| | AGATGTTGATAATCCGCATCTTATGGGTTGGGATTACCCTAAGTGTGATAGAGCTATGCCTAATATG |
| | TGTAGAATCTTCGCTTCACTCATATTAGCTCGTAAACATGGCACTTGTTGTACTACAAGGGACAGAT |
| | TTTATCGCTTGGCAAATGAGTGTGCTCAGGTGCTAAGCGAATATGTTCTATGTGGTGGTGGTTACTA |
| | CGTCAAACCTGGAGGTACCAGTAGCGGAGATGCCACCACTGCATATGCCAATAGTGTCTTTAACATT |
| | TTGCAGGCGACAACTGCTAATGTCAGTGCACTTATGGGTGCTAATGGCAACAAGATTGTTGACAAAG |
| | AAGTTAAAGACATGCAGTTTGATTTGTATGTCAATGTTTACAGGAGCACTAGCCCAGACCCCAAATT |
| | TGTTGATAAATACTATGCTTTTCTTAATAAGCACTTTTCTATGATGATACTGTCTGATGACGGTGTC |
| | GTTTGCTATAATAGTGATTATGCAGCTAAGGGTTACATTGCTGGAATACAGAATTTTAAGGAAACGC |
| | TGTATTATCAGAACAATGTCTTTATGTCTGAAGCTAAATGCTGGGTGGAAACCGATCTGAAGAAAGG |
| | ACCACATGAATTCTGTTCACAGCATACGCTTTATATTAAGGATGGCGACGATGGTTACTTCCTTCCT |
| | TATCCAGACCCTTCAAGAATTTTGTCTGCCGGTTGCTTTGTAGATGATATCGTTAAGACTGACGGTA |
| | CACTCATGGTAGAGCGGTTTGTGTCTTTGGCTATAGATGCTTACCCTCTCACAAAGCATGAAGATAT |
| | AGAATACCAGAATGTATTCTGGGTCTACTTACAGTATATAGAAAAACTGTATAAAGACCTTACAGGA |
| | CACATGCTTGACAGTTATTCTGTCATGCTATGTGGTGATAATTCTGCTAAGTTTTGGGAAGAGGCAT |
| | TCTACAGAGATCTCTATAGTTCGCCTACCACTTTGCAGGCTGTCGGTTCATGCGTTGTATGCCATTC |
| | ACAGACTTCCCTACGCTGTGGGACATGCATCCGTAGACCATTTCTCTGCTGTAAATGCTGCTATGAT |
| | CATGTTATAGCAACTCCACATAAGATGGTTTTGTCTGTTTCTCCTTACGTTTGTAATGCCCCTGGTT |
| | GTGGCGTTTCAGACGTTACTAAGCTATATTTAGGTGGTATGAGCTACTTTTGTGTAGATCATAGACC |
| | TGTGTGTAGTTTTCCACTTTGCGCTAATGGTCTTGTATTCGGCTTATACAAGAATATGTGCACAGGT |
| | AGTCCTTCTATAGTTGAATTTAATAGGTTGGCTACCTGTGACTGGACTGAAAGTGGTGATTACACCC |
| | TTGCCAATACTACAACAGAACCACTTAAACTTTTTGCTGCTGAGACTTTACGTGCCACTGAAGAGGC |
| | GTCTAAGCAGTCTTATGCTATTGCCACCATCAAAGAAATTGTTGGTGAGCGCCAACTATTACTTGTG |
| | TGGGAGGCTGGCAAGTCCAAACCACCACTCAATCGTAATTATGTTTTTACTGGTTATCATATAACCA |
| | AAAATAGTAAAGTGCAGCTCGGTGAGTACATCTTCGAGCGCATTGATTATAGTGATGCTGTATCCTA |
| | CAAGTCTAGTACAACGTATAAACTGACTGTAGGTGACATCTTCGTACTTACCTCTCACTCTGTGGCT |
| | ACCTTGACGGCGCCCACAATTGTGAATCAAGAGAGGTATGTTAAAATTACTGGGTTGTACCCAACCA |
| | TTACGGTACCTGAAGAGTTCGCAAGTCATGTTGCCAACTTCCAAAAATCAGGTTATAGTAAATATGT |
| | CACTGTTCAGGGACCACCTGGCACTGGCAAAAGTCATTTTGCTATAGGGTTAGCGATTTACTACCCT |
| | ACAGCACGTGTTGTTTATACAGCATGTTCACACGCAGCTGTTGATGCTTTGTGTGAAAAAGCTTTTA |
| | AATATTTGAACATTGCTAAATGTTCCCGTATCATTCCTGCAAAGGCACGTGTTGAGTGCTATGACAG |
| | GTTTAAAGTTAATGAGACAAATTCTCAATATTTGTTTAGTACTATTAATGCTCTACCAGAAACTTCT |
| | GCCGATATTCTGGTGGTTGATGAGGTTAGTATGTGCACTAATTATGATCTTTCAATTATTAATGCAC |
| | GTATTAAAGCTAAGCACATTGTCTATGTAGGAGATCCAGCACAGTTGCCAGCTCCTAGGACTTTGTT |
| | GACTAGAGGCACATTGGAACCAGAAAATTTCAATAGTGTCACTAGATTGATGTGTAACTTAGGTCCT |
| | GACATATTTTTAAGTATGTGCTACAGGTGTCCTAAGGAAATAGTAAGCACTGTGAGCGCTCTTGTCT |
| | ACAATAATAAATTGTTAGCCAAGAAGGAGCTTTCAGGCCAGTGCTTTAAAATACTCTATAAGGGCAA |
| | TGTGACGCATGATGCTAGCTCTGCCATTAATAGACCACAACTCACATTTGTGAAGAATTTTATTACT |
| | GCCAATCCGGCATGGAGTAAGGCAGTCTTTATTTCGCCTTATAATTCACAGAATGCTGTGGCTCGTT |
| | CAATGCTGGGTCTTACTACTCAGACTGTTGATTCCTCACAGGGTTCAGAATACCAGTATGTTATCTT |
| | CTGTCAAACAGCAGATACGGCACATGCTAACAACATTAACAGATTTAATGTTGCAATCACTCGTGCC |
| | CAAAAAGGTATTCTTTGTGTTATGACATCTCAGGCACTCTTTGAGTCCTTAGAGTTTACTGAATTGT |
| | CTTTTACTAATTACAAGCTCCAGTCTCAGATTGTAACTGGCCTTTTTAAAGATTGCTCTAGAGAAAC |
| | TTCTGGCCTCTCACCTGCTTATGCACCAACATACGTTAGTGTTGATGACAAGTATAAGACGAGTGAT |
| | GAGCTTTGCGTGAATCTTAATTTACCCGCAAATATCCCATACTCTCGTGTTATTTCCAGGATGGGCT |
| | TTAAACTCGATGCAACAGTTCCTGGATATCCTAAGCTTTTCATTACTCGTGAAGAGGCTGTAAGGCA |
| | AGTTCGAAGCTGGATAGGCTTCGATGTTGAGGGTGCTCATGCTTCCCGTAATGCATGTGGCACCAAT |
| | GTGCCTCTACAATTAGGATTTTCAACTGGTGTGAACTTTGTTGTTCAGCCAGTTGGTGTTGTAGACA |
| | CTGAGTGGGGTAACATGTTAACGGGCATTGCTGCCCGTCCTCCACCAGGTGAACAGTTTAAGCACCT |
| | CGTGCCTCTTATGCATAAGGGGGCTGCGTGGCCTATTGTTAGACGACGTATAGTGCAAATGTTGTCA |
| | GACACTTTAGACAAATTGTCTGATTACTGTACGTTTGTTTGTTGGGCTCATGGCTTTGAATTAACGT |
| | CTGCATCATACTTTTGCAAGATAGGTAAGGAACAGAAGTGTTGCATGTGCAATAGACGCGCTGCAGC |
| | GTACTCTTCACCTCTGCAATCTTATGCCTGCTGGACTCATTCCTGCGGTTATGATTATGTCTACAAC |
| | CCTTTCTTTGTCGATGTTCAACAGTGGGGTTATGTAGGCAATCTTGCTACTAATCACGATCGTTATT |
| | GCTCTGTCCATCAAGGAGCTCATGTGGCTTCTAATGATGCAATAATGACTCGTTGTTTAGCTATTCA |
| | TTCTTGTTTTATAGAACGTGTGGATTGGGATATAGAGTATCCTTATATCTCACATGAAAAGAAATTG |
| | AATTCCTGTTGTAGAATCGTTGAGCGCAACGTCGTACGTGCTGCTCTTCTTGCCGGTTCATTTGACA |
| | AAGTCTATGATATTGGCAATCCTAAAGGAATTCCTATTGTTGATGACCCTGTGGTTGATTGGCATTA |
| | TTTTGATGCACAGCCCTTGACCAGAAAGGTACAACAGCTTTTCTATACAGAGGACATGGCCTCAAGA |
| | TTTGCTGATGGGCTCTGCTTATTTTGGAACTGTAATGTACCAAAATATCCTAATAATGCAATTGTAT |
| | GCAGGTTTGACACACGTGTGCATTCTGAGTTCAATTTGCCAGGTTGTGATGGCGGTAGTTTGTATGT |
| | TAACAAGCACGCTTTTCATACACCAGCATATGATGTGAGTGCATTCCGTGATCTGAAACCTTTACCA |
| | TTCTTTTATTATTCTACTACACCATGTGAAGTGCATGGTAATGGTAGTATGATAGAGGATATTGATT |
| | ATGTACCCCTAAAATCTGCAGTCTGTATTACAGCTTGTAATTTAGGGGGCGCTGTTTGTAGGAAGCA |
| | TGCTACAGAGTACAGAGAGTATATGGAAGCATATAATCTTGTCTCTGCATCAGGTTTCCGCCTTTGG |
| | TGTTATAAGACCTTTGATATTTATAATCTCTGGTCTACTTTTACAAAAGTTCAAGGTTTGGAAAACA |
| | TTGCTTTTAATGTTGTTAAACAAGGCCATTTTATTGGTGTTGAGGGTGAACTACCTGTAGCTGTAGT |
| | CAATGATAAGATCTTCACCAAGAGTGGCGTTAATGACATTTGTATGTTTGAGAATAAAACCACTTTG |
| | CCTACTAATATAGCTTTTGAACTCTATGCTAAGCGTGCTGTACGCTCGCATCCCGATTTCAAATTGC |
| | TACACAATTTACAAGCAGACATTTGCTACAAGTTCGTCCTTTGGGATTATGAACGTAGCAATATTTA |
| | TGGTACTGCTACTATTGGTGTATGTAAGTACACTGATATTGATGTTAATTCAGCTTTGAATATATGT |
| | TTTGACATACGCGATAATGGTTCATTGGAGAAGTTCATGTCTACTCCCAATGCCATCTTTATTTCTG |
| | ATAGAAAAATTAAGAAATACCCTTGTATTGTAGGTCCTGATTATGCTTACTTCAATGGTGCTATCAT |
| | CCGTGATAGTGATGTTGTTAAACAACCAGTGAAGTTCTACTTGTATAAGAAAGTCAATAATGAGTTT |
| | ATTGATCCTACTGAGTGTATTTACACTCAGAGTCGCTCTTGTAGTGACTTCCTACCCCTGTCTGACA |
| | TGGAGAAAGACTTTCTATCTTTTGATAGTGATGTTTTCATTAAGAAGTATGGCTTGGAAAACTATGC |
| | TTTTGAGCACGTAGTCTATGGAGACTTCTCTCATACTACGTTAGGCGGTCTTCACTTGCTTATTGGT |
| | TTATACAAGAAGCAACAGGAAGGTCATATTATTATGGAAGAAATGCTAAAAGGTAGCTCAACTATTC |
| | ATAACTATTTTATTACTGAGACTAACACAGCGGCTTTTAAGGCGGTGTGTTCTGTTATAGATTTAAA |
| | GCTTGACGACTTTGTTATGATTTTAAAGAGTCAAGACCTTGGCGTAGTATCCAAGGTTGTCAAGGTT |
| | TCTATTGACTTAACAATGATTGAGTTTATGTTATGGTGTAAGGATGGACAGGTTCAAACCTTCTACC |
| | CTCGACTCCAGGCTTCTGCAGATTGGAAACCTGGTCATGCAATGCCATCCCTCTTTAAAGTTCAAAA |
| | TGTAAACCTTGAACGTTGTGAGCTTGCTAATTACAAGCAATCTATTCCTATGCCTCGCGGTGTGCAC |
| | ATGAACATCGCTAAATATATGCAATTGTGCCAGTATTTAAATACTTGCACATTAGCCGTGCCTGCCA |
| | ATATGCGTGTTATACATTTTGGCGCTGGTTCTGATAAAGGTATCGCTCCTGGTACATCAGTTTTACG |
| | ACAGTGGCTTCCTACAGATGCCATTATTATAGATAATGATTTAAATGAGTTCGTGTCAGATGCTGAC |
| | ATAACTTTATTTGGAGATTGTGTAACTGTACGTGTCGGCCAACAAGTGGATCTTGTTATTTCCGACA |
| | TGTATGATCCTACTACTAAGAATGTAACAGGTAGTAATGAGTCAAAGGCTTTATTCTTTACTTACCT |
| | GTGTAACCTCATTAATAATAATCTTGCTCTTGGTGGGTCTGTTGCTATTAAAATAACAGAACACTCT |
| | TGGAGCGTTGAACTTTATGAACTTATGGGAAAATTTGCTTGGTGGACTGTTTTCTGCACCAATGCAA |
| | ATGCATCCTCATCTGAAGGATTCCTCTTAGGTATTAATTACTTGGGTACTATTAAAGAAAATATAGA |
| | TGGTGGTGCTATGCACGCCAACTATATATTTTGGAGAAATTCCACTCCTATGAATCTGAGTACTTAC |
| | TCACTTTTTGATTTATCCAAGTTTCAATTAAAATTAAAAGGAACACCAGTTCTTCAATTAAAGGAGA |
| | GTCAAATTAACGAACTCGTAATATCTCTCCTGTCGCAGGGTAAGTTACTTATCCGTGACAATGATAC |
| | ACTCAGTGTTTCTACTGATGTTCTTGTTAACACCTACAGAAAGTTACGTTGATGTAGGGCCAGATTC |
| | TGTTAAGTCTGCTTGTATTGAGGTTGATATACAACAGACTTTCTTTGATAAAACTTGGCCTAGGCCA |
| | ATTGATGTTTCTAAGGCTGACGGTATTATATACCCTCAAGGCCGTACATATTCTAACATAACTATCA |
| | CTTATCAAGGTCTTTTTCCCTATCAGGGAGACCATGGTGATATGTATGTCTACTCTGCAGGACATGC |
| | TACAGGCATAACTCCACAAAAGTTGTTTGTAGCTAACTATTCTCAGGACGTCAAACAGTTTGCTAAT |
| | GGGTTTGTCGTCCGTATAGGAGCAGCTGCCAATTCCACTGGCACTGTTATTATTAGCCCATCTACCA |
| | GCGCTACTATACGAAAAATTTACCCTGCTTTTATGCTGGGTTCTTCAGTTGGTAATTTTTCAGATGG |
| | TAAAATGGGCCGCTTCTTCAATCATACTCTAGTTCTTTTGCCCGATGGATGTGGCACTTTACTTAGA |
| | GCTTTTTATTGTATTCTAGAGCCTCGCTCTGGAAATCATTGTCCTGCTGGCAATTCCTATACTTCTT |
| | TTGCCACTTATCACACTCCTGCAACAGATTGTTCTGATGGCAATTACAATCGTAATGCCAGTCTGAA |
| | CTCTTTTAAGGAGTATTTTAATTTACGTAACTGCACCTTTATGTACACTTATAACATTACCGAAGAT |
| | GAGATTTTAGAGTGGTTTGGCATCACACAAACTGCTCAAGGTGTTCACCTCTTCTCATCTCGGTATG |
| | TTGATTTGTACGGCGGCAATATGTTTCAATTTGCCACCTTGCCTGTTTATGATACTATTAAGTATTA |
| | TTCTATCATTCCTCACAGTATTCGTTCTATCCAAAGTGATAGAAAAGCTTGGGCTGCCTTCTACGTA |
| | TATAAACTTCAACCGTTAACTTTCCTGTTGGATTTTTCTGTTGATGGTTATATACGCAGAGCTATAG |
| | ACTGTGGTTTTAATGATTTGTCACAACTCCACTGCTCATATGAATCCTTCGATGTTGAATCTGGAGT |
| | TTATTCAGTTTCGTCTTTCGAAGCAAAACCTTCTGGCTCAGTTGTGGAACAGGCTGAAGGTGTTGAA |
| | TGTGATTTTTCACCTCTTCTGTCTGGCACACCTCCTCAGGTTTATAATTTCAAGCGTTTGGTTTTTA |
| | CCAATTGCAATTATAATCTTACCAAATTGCTTTCACTTTTTTCTGTGAATGATTTTACTTGTAGTCA |
| | AATATCTCCAGCAGCAATTGCTAGCAACTGTTATTCTTCACTGATTTTGGATTATTTTTCATACCCA |
| | CTTAGTATGAAATCCGATCTCAGTGTTAGTTCTGCTGGTCCAATATCCCAGTTTAATTATAAACAGT |
| | CCTTTTCTAATCCCACATGTTTGATTTTAGCGACTGTTCCTCATAACCTTACTACTATTACTAAGCC |
| | TCTTAAGTACAGCTATATTAACAAGTGCTCTCGTCTTCTTTCTGGTGATCGTACTGAAGTACCTCAG |
| | TTAGTGAACGCTAATCAATACTCACCCTGTGTATCCATTGTCCCATCCACTGTGTGGGAAGACGGTG |
| | ATTATTATAGGAAACAACTATCTCCACTTGAAGGTGGTGGCTGGCTTGTTGCTAGTGGCTCAACTGT |
| | TGCCATGACTGAGCAATTACAGATGGGCTTTGGTATTACAGTTCAATATGGTACAGACACCAATAGT |
| | GTTTGCCCCAAGCTTGAATTTGCTAATGACACAAAAATTGCCTCTCAATTAGGCAATTGCGTGGAAT |
| | ATTCCCTCTATGGTGTTTCGGGCCGTGGTGTTTTTCAGAATTGCACAGCTGTAGGTGTTCGACAGCA |
| | GCGCTTTGTTTATGATGCGTACCAGAATTTAGTTGGCTATTATTCTGATGATGGCAACTACTACTGT |
| | TTGCGTGCTTGTGTTAGTGTTCCTGTTTCTGTCATCTATGATAAAGAAACTAAAACCCACGCTACTC |
| | TATTTGGTAGTGTTGCATGTGAACACATTTCCTCTACCATGTCTCAATACTCCCGTTCTACGCGATC |
| | AATGCTTAAACGGCGAGATTCTACATATGGTCCCCTTCAGACACCTGTTGGTTGTGTCCTAGGACTT |
| | GTTAATTCCTCTTTGTTCGTAGAGGACTGCAAGTTGCCTCTTGGTCAATCTCTCTGTGCTCTTCCTG |
| | ACACACCTAGTACTCTCACACCTCGCAGTGTGCGCTCTGTTCCAGGTGAAATGCGCTTGGCATCCAT |
| | TGCTTTTAATCATCCTATTCAGGTTGATCAACTTAATAGTAGTTATTTTAAATTAAGTATACCTACT |
| | AATTTTTCCTTTGGTGTGACTCAGGAGTATATTCAGACAACCATTCAGAAAGTTACTGTTGATTGTA |
| | AACAGTACGTTTGCAATGGTTTCCAGAAGTGTGAGCAATTACTGCGCGAGTATGGCCAGTTTTGTTC |
| | CAAAATAAACCAGGCTCTCCATGGTGCCAATTTACGCCAGGATGATTCTGTACGTAATTTGTTTGCG |
| | AGCGTGAAAAGCTCTCAATCATCTCCTATCATACCAGGTTTTGGAGGTGACTTTAATTTGACACTTC |
| | TAGAACCTGTTTCTATATCTACTGGCAGTCGTAGTGCACGTAGTGCTATTGAGGATTTGCTATTTGA |
| | CAAAGTCACTATAGCTGATCCTGGTTATATGCAAGGTTACGATGATTGTATGCAGCAAGGTCCAGCA |
| | TCAGCTCGTGATCTTATTTGTGCTCAATATGTGGCTGGTTATAAAGTATTACCTCCTCTTATGGATG |
| | TTAATATGGAAGCCGCGTACACTTCATCTTTGCTTGGCAGCATAGCAGGTGTTGGCTGGACTGCTGG |
| | CTTATCCTCCTTTGCTGCTATTCCATTTGCACAGAGTATTTTTTATAGGTTAAACGGTGTTGGCATT |
| | ACTCAACAGGTTCTTTCAGAGAACCAAAAGCTTATTGCCAATAAGTTTAATCAGGCTCTGGGAGCTA |
| | TGCAAACAGGCTTCACTACAACTAATGAAGCTTTTCGGAAGGTTCAGGATGCTGTGAACAACAATGC |
| | ACAGGCTCTATCCAAATTAGCTAGCGAGCTATCTAATACTTTTGGTGCTATTTCCGCATCTATTGGA |
| | GACATCATACAACGTCTTGATGTTCTCGAACAGGACGCCCAAATAGACAGACTTATTAATGGCCGTT |
| | TGACAACACTAAATGCTTTTGTTGCACAGCAGCTTGTTCGTTCCGAATCAGCTGCTCTTTCCGCTCA |
| | ATTGGCTAAAGATAAAGTCAATGAGTGTGTCAAGGCACAATCCAAGCGTTCTGGATTTTGCGGTCAA |
| | GGCACACATATAGTGTCCTTTGTTGTAAATGCCCCTAATGGCCTTTACTTTATGCATGTTGGTTATT |
| | ACCCTAGCAACCACATTGAGGTTGCTTCTGCTTATGGTCTTTGCGATGCAGCTAACCCTACTAATTG |
| | TATAGCCCCTGTTAATGGCTACTTTATTAAAACTAATAACACTAGTATTGTTGATGAGTGGTCATAT |
| | ACTGGCTCGTCCTTCTATGCACCTGAGCCCATCACCTCTCTTAATACTAAGTATGTTGCACCACAGG |
| | TGACATACCAAAACATTTCTACTAACCTCCCTCCTCCTCTTCTCGGCAATTCCACCGGGATTGACTT |
| | CCAAGATGAGTTGGATGAGTTTTTCAAAAATGTTAGCACCAGTATACCTAATTTTGGTTCTCTAACA |
| | CAGATTAATACTACATTACTCGATCTTACCTACGAGATGTTGTCTCTTCAACAAGTTGTTAAAGCCC |
| | TTAATGAGTCTTACATAGACCTTAAAGAGCTTGGCAATTATACTTATTACAACAAATGGCCGTGGTA |
| | CATTTGGCTTGGTTTCATTGCTGGGCTTGTTGCCTTAGCTCTATGCGTCTTCTTCATACTGTGCTGC |
| | ACTGGTTGTGGCACAAACTGTATGGGAAAACTTAAGTGTAATCGTTGTTGTGATAGATACGAGGAAT |
| | ACGACCTCGAGCCGCATAAGGTTCATGTTCACTAATTAACGAACTATCAATGAGAGTTCAAAGACCA |
| | CCCACTCTCTTGTTAGTGTTCTCACTCTCTTTTTTGGTCACTGCATTTTCAAAACCTCTCTATGTAC |
| | CTGAGCATTGTCAGAATTATTCTGGTTGCATGCTTAGGGCTTGTATTAAAACTGCCCAAGCTGATAC |
| | AGCTGGTCTTTATACAAATTTTCGAATTGACGTCCCATCTGCAGAATCAACTGGTACTCAATCAGTT |
| | TCTGTCGATCGTGAGTCTACTTCAACTCATGATGGTCTTACCGAACATGTTACTAGTGTGAATCTTT |
| | TTGACGTTGGTTACTCAGTTAATTAACGAACTCTATGGATTACGTGTCTCTGCTTAATCAAATTTGG |
| | CAGAAGTACCTTAATTCACCGTATACTACTTGTTTGTATATCCCTAAACCCACAGCTAAGTATACAC |
| | CTTTAGTTGGCACTTCATTGCACCCTGTGCTGTGGAACTGTCAGCTATCCTTTGCTGGTTATACTGA |
| | ATCTGCTGTTAATTCTACAAAAGCTTTGGCCAAACAGGACGCAGCTCAGCGAATCGCTTGGTTGCTA |
| | CATAAGGATGGAGGAATCCCTGACGGATGTTCCCTCTACCTCCGGCACTCAAGTTTATTCGCGCAAA |
| | GCGAGGAAGAGGAGTCATTCTCCAACTAAGAAACTGCGCTACGTTAAGCGTAGATTTTCTCTTCTGC |
| | GCCCTGAAGACCTTAGTGTTATTGTCCAACCAACACACTATGTCAGGGTTACATTTTCAGACCCCAA |
| | CATGTGGTATCTACGTTCGGGTCATCATTTACACTCAGTTCACAATTGGCTTAAACCTTATGGCGGC |
| | CAACCTGTTTCTGAGTACCATATTACTCTAGCTTTGCTAAATCTCACTGATGAAGATTTAGCTAGAG |
| | ATTTTTCACCCATTGCGCTCTTTTTGCGCAATGTCAGATTTGAGCTACATGAGTTCGCCTTGCTGCG |
| | CAAAACTCTTGTTCTTAATGCATCAGAGATCTACTGTGCTAACATACATAGATTTAAGCCTGTGTAT |
| | AGAGTTAACACGGCAATCCCTACTATTAAGGATTGGCTTCTCGTTCAGGGATTTTCCCTTTACCATA |
| | GTGGCCTCCCTTTACATATGTCAATCTCTAAATTGCATGCACTGGATGATGTTACTCGCAATTACAT |
| | CATTACAATGCCATGCTTTAGAACTTATCCTCAACAAATGTTTGTTACTCCTTTGGCCGTAGATGTT |
| | GTCTCCATACGGTCTTCCAATCAGGGTAATAAACAAATTGTTCATTCTTACCCCATTTTACATCATC |
| | CAGGATTTTAACGAACTATGGCTTTCTCGGCGTCTTTATTTAAACCCGTCCAGCTAGTCCCAGTTTC |
| | CCCTGCATTTCATCGCATTGAGTCTACTGACTCTATTGTTTTCACATACATTCCTGCTAGCGGCTAT |
| | GTAGCTGCTTTAGCTGTCAATGTGTGTCTCATTCCCCTATTATTACTGCTACGTCAAGATACTTGTC |
| | GTCGCAGCATTATCAGAACTATGGTTCTCTATTTCCTTGTTCTTTATAACTTTTTATTAGCCATTGT |
| | ACTAGTCAATGGTGTACATTATCCAACTGGAAGTTGCCTGATAGCCTTCTTAGTTATCCTCATAATA |
| | CTTTGGTTTGTAGATAGAATTCGTTTCTGTCTCATGCTGAATTCCTACATTCCACTGTTTGACATGC |
| | GTTCTCACTTTATTCGTGTTAGTACAGTTTCTTCTCATGGTATGGTCCCTGTCATACACACCAAACC |
| | ATTATTTATTAGAAACTTCGATCAGCGTTGCAGCTGTTCTCGTTGTTTTTATTTGCACTCTTCTACT |
| | TATATAGAGTGCACTTATATTAGCCGTTTTAGTAAGATTAGCCTAGTTTCTGTAACTGACTTCTCCT |
| | TAAACGGCAATGTTTCCACTGTTTTCGTGCCTGCAACGCGCGATTCAGTTCCTCTTCACATAATCGC |
| | CCCGAGCTCGCTTATCGTTTAAGCAGCTCTGCGCTACTATGGGTCCCGTGTAGAGGCTAATCCATTA |
| | GTCTCTCTTTGGACATATGGAAAACGAACTATGTTACCCTTTGTCCAAGAACGAATAGGGTTGTTCA |
| | TAGTAAACTTTTTCATTTTTACCGTAGTATGTGCTATAACACTCTTGGTGTGTATGGCTTTCCTTAC |
| | GGCTACTAGATTATGTGTGCAATGTATGACAGGCTTCAATACCCTGTTAGTTCAGCCCGCATTATAC |
| | TTGTATAATACTGGACGTTCAGTCTATGTAAAATTCCAGGATAGTAAACCCCCTCTACCACCTGACG |
| | AGTGGTTTTAACGAACTCCTTCATAATGTCTAATATGACGCAACTCACTGAGGCGCAGATTATTGCC |
| | ATTATTAAAGACTGGAACTTTGCATGGTCCCTGATCTTTCTCTTAATTACTATCGTACTACAGTATG |
| | GATACCCATCCCGTAGTATGACTGTCTATGTCTTTAAAATGTTTGTTTTATGGCTCCTATGGCCATC |
| | TTCCATGGCGCTATCAATATTTAGCGCCATTTATCCAATTGATCTAGCTTCCCAGATAATCTCTGGC |
| | ATTGTATCAGCTGTTTCAGCTATGATGTGGATTTCCTACTTTGTGCAGAGTATCCGGCTGTTTATGA |
| | GAACTGGATCATGGTGGTCATTCAATCCTGAGACTAATTGCCTTTTGAACGTTCCAATTGGTGGTAC |
| | AACTGTCGTACGTCCACTCGTAGAGGACTCTACCAGTGTAACTGCTGTTGTAACCAATGGTCACCTC |
| | AAAATGGCTGGCATGCATTTCGGTGCTTGTGACTACGACAGACTTCCTAATGAAGTCACCGTGGCCA |
| | AACCCAATGTGCTGATTGCTTTAAAAATGGTGAAGCGGCAAAGCTACGGAACTAATTCCGGCGTTGC |
| | CATTTACCATAGATATAAGGCAGGTAATTACAGGAGTCCGCCTATTACGGCGGATATTGAACTTGCA |
| | TTGCTTCGAGCTTAGGCTCTTTAGTAAGAGTATCTTAATTGATTTTAACGAATCTCAATTTCATTGT |
| | TATGGCATCCCCTGCTGCACCTCGTGCTGTTTCCTTTGCCGATAACAATGATATAACAAATACAAAC |
| | ATGTCTCGAGGTAGAGGACGTAATCCAAAACCACGAGCTGCACCAAATAACACTGTCTCTTGGTACA |
| | CTGGGCTTACCCAACACGGGAAAGTCCCTCTTACCTTTCCACCTGGGCAGGGTGTACCTCTTAATGC |
| | CAATTCCACCCCTGCGCAAAATGCTGGGTATTGGCGGAGACAGGACAGAAAAATTAATACCGGGAAT |
| | GGAATTAAGCAACTGGCTCCCAGGTGGTACTTCTACTACACTGGAACTGGACCCGAAGCAGCACTCC |
| | CATTCCGGGCTGTTAAGGATGGCATCGTTTGGGTCCATGAAGATGGCGCCACTGATGCTCCTTCAAC |
| | TTTTGGGACGCGGAACCCTAACAATGATTCAGCTATTGTTACACAATTCGCGCCCGGTACTAAGCTT |
| | CCTAAAAACTTCCACATTGAGGGGACTGGAGGCAATAGTCAATCATCTTCAAGAGCCTCTAGCGTAA |
| | GCAGAAACTCTTCCAGATCTAGTTCACAAGGTTCAAGATCAGGAAACTCTACCCGCGGCACTTCTCC |
| | AGGTCCATCTGGAATCGGAGCAGTAGGAGGTGATCTACTTTACCTTGATCTTCTGAACAGACTACAA |
| | GCCCTTGAGTCTGGCAAAGTAAAGCAATCGCAGCCAAAAGTAATCACTAAGAAAGATGCTGCTGCTG |
| | CTAAAAATAAGATGCGCCACAAGCGCACTTCCACCAAAAGTTTCAACATGGTGCAAGCTTTTGGTCT |
| | TCGCGGACCAGGAGACCTCCAGGGAAACTTTGGTGATCTTCAATTGAATAAACTCGGCACTGAGGAC |
| | CCACGTTGGCCCCAAATTGCTGAGCTTGCTCCTACAGCCAGTGCTTTTATGGGTATGTCGCAATTTA |
| | AACTTACCCATCAGAACAATGATGATCATGGCAACCCTGTGTACTTCCTTCGGTACAGTGGAGCCAT |
| | TAAACTTGACCCAAAGAATCCCAACTACAATAAGTGGTTGGAGCTTCTTGAGCAAAATATTGATGCC |
| | TACAAAACCTTCCCTAAGAAGGAAAAGAAACAAAAGGCACCAAAAGAAGAATCAACAGACCAAATGT |
| | CTGAACCTCCAAAGGAGCAGCGTGTGCAAGGTAGCATCACTCAGCGCACTCGCACCCGTCCAAGTGT |
| | TCAGCCTGGTCCAATGATTGATGTTAACACTGATTAGTGTCACTCAAAGTAACAAGATCGCGGCAAT |
| | CGTTTGTGTTTGGTAACCCCATCTCACCATCGCTTGTCCACTCTTGCACAGAATGGAATCATGTTGT |
| | AATTACAGTGCAATAAGGTAATTATAACCCATTTAATTGATAGCTATGCTTTATTAAAGTGTGTAGC |
| | TGTAGAGAGAATGTTAAAGACTGTCACCTCTGCGTGATTGCAAGTGAACAGTGCCCCCCGGGAAGAG |
| | CTCTACAGTGTGAAATGTAAATAAAAATAGCTATTATTCAATTAGATTAGGCTAATTAGATGATTTG |
| | CAAAAAAAAAAAA |
| 5 | GenBank Accession No. MT263402.1 (Severe acute respiratory syndrome |
| | coronavirus 2 isolate SARS-CoV-2/human/USAAVA-UW322/2020, complete |
| | genome) |
| | CCTTTAAACTTTCGATCTCTTGTAGATCTGTTCTCTAAACGAACTTTAAAATCTGTGTGGCTGTCAC |
| | TCGGCTGCATGCTTAGTGCACTCACGCAGTATAATTAATAACTAATTACTGTCGTTGACAGGACACG |
| | AGTAACTCGTCTATCTTCTGCAGGCTGCTTACGGTTTCGTCCGTGTTGCAGCCGATCATCAGCACAT |
| | CTAGGTTTTGTCCGGGTGTGACCGAAAGGTAAGATGGAGAGCCTTGTCCCTGGTTTCAACGAGAAAA |
| | CACACGTCCAACTCAGTTTGCCTGTTTTACAGGTTCGCGACGTGCTCGTACGTGGCTTTGGAGACTC |
| | CGTGGAGGAGGTCTTATCAGAGGCACGTCAACATCTTAAAGATGGCACTTGTGGCTTAGTAGAAGTT |
| | GAAAAAGGCGTTTTGCCTCAACTTGAACAGCCCTATGTGTTCATCAAACGTTCGGATGCTCGAACTG |
| | CACCTCATGGTCATGTTATGGTTGAGCTGGTAGCAGAACTCGAAGGCATTCAGTACGGTCGTAGTGG |
| | TGAGACACTTGGTGTCCTTGTCCCTCATGTGGGCGAAATACCAGTGGCTTACCGCAAGGTTCTTCTT |
| | CGTAAGAACGGTAATAAAGGAGCTGGTGGCCATAGTTACGGCGCCGATCTAAAGTCATTTGACTTAG |
| | GCGACGAGCTTGGCACTGATCCTTATGAAGATTTTCAAGAAAACTGGAACACTAAACATAGCAGTGG |
| | TGTTACCCGTGAACTCATGCGTGAGCTTAACGGAGGGGCATACACTCGCTATGTCGATAACAACTTC |
| | TGTGGCCCTGATGGCTACCCTCTTGAGTGCATTAAAGACCTTCTAGCACGTGCTGGTAAAGCTTCAT |
| | GCACTTTGTCCGAACAACTGGACTTTATTGACACTAAGAGGGGTGTATACTGCTGCCGTGAACATGA |
| | GCATGAAATTGCTTGGTACACGGAACGTTCTGAAAAGAGCTATGAATTGCAGACACCTTTTGAAATT |
| | AAATTGGCAAAGAAATTTGACACCTTCAATGGGGAATGTCCAAATTTTGTATTTCCCTTAAATTCCA |
| | TAATCAAGACTATTCAACCAAGGGTTGAAAAGAAAAAGCTTGATGGCTTTATGGGTAGAATTCGATC |
| | TGTCTATCCAGTTGCGTCACCAAATGAATGCAACCAAATGTGCCTTTCAACTCTCATGAAGTGTGAT |
| | CATTGTGGTGAAACTTCATGGCAGACGGGCGATTTTGTTAAAGCCACTTGCGAATTTTGTGGCACTG |
| | AGAATTTGACTAAAGAAGGTGCCACTACTTGTGGTTACTTACCCCAAAATGCTGTTGTTAAAATTTA |
| | TTGTCCAGCATGTCACAATTCAGAAGTAGGACCTGAGCATAGTCTTGCCGAATACCATAATGAATCT |
| | GGCTTGAAAACCATTCTTCGTAAGGGTGGTCGCACTATTGCCTTTGGAGGCTGTGTGTTCTCTTATG |
| | TTGGTTGCCATAACAAGTGTGCCTATTGGGTTCCACGTGCTAGCGCTAACATAGGTTGTAACCATAC |
| | AGGTGTTGTTGGAGAAGGTTCCGAAGGTCTTAATGACAACCTTCTTGAAATACTCCAAAAAGAGAAA |
| | GTCAACATCAATATTGTTGGTGACTTTAAACTTAATGAAGAGATCGCCATTATTTTGGCATCTTTTT |
| | CTGCTTCCACAAGTGCTTTTGTGGAAACTGTGAAAGGTTTGGATTATAAAGCATTCAAACAAATTGT |
| | TGAATCCTGTGGTAATTTTAAAGTTACAAAAGGAAAAGCTAAAAAAGGTGCCTGGAATATTGGTGAA |
| | CAGAAATCAATACTGAGTCCTCTTTATGCATTTGCATCAGAGGCTGCTCGTGTTGTACGATCAATTT |
| | TCTCCCGCACTCTTGAAACTGCTCAAAATTCTGTGCGTGTTTTACAGAAGGCCGCTATAACAATACT |
| | AGATGGAATTTCACAGTATTCACTGAGACTCATTGATGCTATGATGTTCACATCTGATTTGGCTACT |
| | AACAATCTAGTTGTAATGGCCTACATTACAGGTGGTGTTGTTCAGTTGACTTCGCAGTGGCTAACTA |
| | ACATCTTTGGCACTGTTTATGAAAAACTCAAACCCGTCCTTGATTGGCTTGAAGAGAAGTTTAAGGA |
| | AGGTGTAGAGTTTCTTAGAGACGGTTGGGAAATTGTTAAATTTATCTCAACCTGTGCTTGTGAAATT |
| | GTCGGTGGACAAATTGTCACCTGTGCAAAGGAAATTAAGGAGAGTGTTCAGACATTCTTTAAGCTTG |
| | TAAATAAATTTTTGGCTTTGTGTGCTGACTCTATCATTATTGGTGGAGCTAAACTTAAAGCCTTGAA |
| | TTTAGGTGAAACATTTGTCACGCACTCAAAGGGATTGTACAGAAAGTGTGTTAAATCCAGAGAAGAA |
| | ACTGGCCTACTCATGCCTCTAAAAGCCCCAAAAGAAATTATCTTCTTAGAGGGAGAAACACTTCCCA |
| | CAGAAGTGTTAACAGAGGAAGTTGTCTTGAAAACTGGTGATTTACAACCATTAGAACAACCTACTAG |
| | TGAAGCTGTTGAAGCTCCATTGGTTGGTACACCAGTTTGTATTAACGGGCTTATGTTGCTCGAAATC |
| | AAAGACACAGAAAAGTACTGTGCCCTTGCACCTAATATGATGGTAACAAACAATACCTTCACACTCA |
| | AAGGCGGTGCACCAACAAAGGTTACTTTTGGTGATGACACTGTGATAGAAGTGCAAGGTTACAAGAG |
| | TGTGAATATCACTTTTGAACTTGATGAAAGGATTGATAAAGTACTTAATGAGAAGTGCTCTGCCTAT |
| | ACAGTTGAACTCGGTACAGAAGTAAATGAGTTCGCCTGTGTTGTGGCAGATGCTGTCATAAAAACTT |
| | TGCAACCAGTATCTGAATTACTTACACCACTGGGCATTGATTTAGATGAGTGGAGTATGGCTACATA |
| | CTACTTATTTGATGAGTCTGGTGAGTTTAAATTGGCTTCACATATGTATTGTTCTTTTTACCCTCCA |
| | GATGAGGATGAAGAAGAAGGTGATTGTGAAGAAGAAGAGTTTGAGCCATCAACTCAATATGAGTATG |
| | GTACTGAAGATGATTACCAAGGTAAACCTTTGGAATTTGGTGCCACTTCTGCTGCTCTTCAACCTGA |
| | AGAAGAGCAAGAAGAAGATTGGTTAGATGATGATAGTCAACAAACTGTTGGTCAACAAGACGGCAGT |
| | GAGGACAATCAGACAACTACTATTCAAACAATTGTTGAGGTTCAACCTCAATTAGAGATGGAACTTA |
| | CACCAGTTGTTCAGACTATTGAAGTGAATAGTTTTAGTGGTTATTTAAAACTTACTGACAATGTATA |
| | CATTAAAAATGCAGACATTGTGGAAGAAGCTAAAAAGGTAAAACCAACAGTGGTTGTTAATGCAGCC |
| | AATGTTTACCTTAAACATGGAGGAGGTGTTGCAGGAGCCTTAAATAAGGCTACTAACAATGCCATGC |
| | AAGTTGAATCTGATGATTACATAGCTACTAATGGACCACTTAAAGTGGGTGGTAGTTGTGTTTTAAG |
| | CGGACACAATCTTGCTAAACACTGTCTTCATGTTGTCGGCCCAAATGTTAACAAAGGTGAAGACATT |
| | CAACTTCTTAAGAGTGCTTATGAAAATTTTAATCAGCACGAAGTTCTACTTGCACCATTATTATCAG |
| | CTGGTATTTTTGGTGCTGACCCTATACATTCTTTAAGAGTTTGTGTAGATACTGTTCGCACAAATGT |
| | CTACTTAGCTGTCTTTGATAAAAATCTCTATGACAAACTTGTTTCAAGCTTTTTGGAAATGAAGAGT |
| | GAAAAGCAAGTTGAACAAAAGATCGCTGAGATTCCTAAAGAGGAAGTTAAGCCATTTATAACTGAAA |
| | GTAAACCTTCAGTTGAACAGAGAAAACAAGATGATAAGAAAATCAAAGCTTGTGTTGAAGAAGTTAG |
| | AACAACTCTGGAAGAAACTAAGTTCCTCACAGAAAACTTGTTACTTTATATTGACATTAATGGCAAT |
| | CTTCATCCAGATTCTGCCACTCTTGTTAGTGACATTGACATCACTTTCTTAAAGAAAGATGCTCCAT |
| | ATATAGTGGGTGATGTTGTTCAAGAGGGTGTTTTAACTGCTGTGGTTATACCTACTAAAAAGGCTGG |
| | TGGCACTACTGAAATGCTAGCGAAAGCTTTGAGAAAAGTGCCAACAGACAATTATATAACCACTTAC |
| | CCGGGTCAGGGTTTAAATGGTTACACTGTAGAGGAGGCAAAGACAGTGCTTAAAAAGTGTAAAAGTG |
| | CCTTTTACATTCTACCATCTATTATCTCTAATGAGAAGCAAGAAATTCTTGGAACTGTTTCTTGGAA |
| | TTTGCGAGAAATGCTTGCACATGCAGAAGAAACACGCAAATTAATGCCTGTCTGTGTGGAAACTAAA |
| | GCCATAGTTTCAACTATACAGCGTAAATATAAGGGTATTAAAATACAAGAGGGTGTGGTTGATTATG |
| | GTGCTAGATTTTACTTTTACACCAGTAAAACAACTGTAGCGTCACTTATCAACACACTTAACGATCT |
| | AAATGAAACTCTTGTTACAATGCCACTTGGCTATGTAACACATGGCTTAAATTTGGAAGAAGCTGCT |
| | CGGTATATGAGATCTCTCAAAGTGCCAGCTACAGTTTCTGTTTCTTCACCTGATGCTGTTACAGCGT |
| | ATAATGGTTATCTTACTTCTTCTTCTAAAACACCTGAAGAACATTTTATTGAAACCATCTCACTTGC |
| | TGGTTCCTATAAAGATTGGTCCTATTCTGGACAATCTACACAACTAGGTATAGAATTTCTTAAGAGA |
| | GGTGATAAAAGTGTATATTACACTAGTAATCCTACCACATTCCACCTAGATGGTGAAGTTATCACCT |
| | TTGACAATCTTAAGACACTTCTTTCTTTGAGAGAAGTGAGGACTATTAAGGTGTTTACAACAGTAGA |
| | CAACATTAACCTCCACACGCAAGTTGTGGACATGTCAATGACATATGGACAACAGTTTGGTCCAACT |
| | TATTTGGATGGAGCTGATGTTACTAAAATAAAACCTCATAATTCACATGAAGGTAAAACATTTTATG |
| | TTTTACCTAATGATGACACTCTACGTGTTGAGGCTTTTGAGTACTACCACACAACTGATCCTAGTTT |
| | TCTGGGTAGGTACATGTCAGCATTAAATCACACTAAAAAGTGGAAATACCCACAAGTTAATGGTTTA |
| | ACTTCTATTAAATGGGCAGATAACAACTGTTATCTTGCCACTGCATTGTTAACACTCCAACAAATAG |
| | AGTTGAAGTTTAATCCACCTGCTCTACAAGATGCTTATTACAGAGCAAGGGCTGGTGAAGCTGCTAA |
| | CTTTTGTGCACTTATCTTAGCCTACTGTAATAAGACAGTAGGTGAGTTAGGTGATGTTAGAGAAACA |
| | ATGAGTTACTTGTTTCAACATGCCAATTTAGATTCTTGCAAAAGAGTCTTGAACGTGGTGTGTAAAA |
| | CTTGTGGACAACAGCAGACAACCCTTAAGGGTGTAGAAGCTGTTATGTACATGGGCACACTTTCTTA |
| | TGAACAATTTAAGAAAGGTGTTCAGATACCTTGTACGTGTGGTAAACAAGCTACAAAATATCTAGTA |
| | CAACAGGAGTCACCTTTTGTTATGATGTCAGCACCACCTGCTCAGTATGAACTTAAGCATGGTACAT |
| | TTACTTGTGCTAGTGAGTACACTGGTAATTACCAGTGTGGTCACTATAAACATATAACTTCTAAAGA |
| | AACTTTGTATTGCATAGACGGTGCTTTACTTACAAAGTCCTCAGAATACAAAGGTCCTATTACGGAT |
| | GTTTTCTACAAAGAAAACAGTTACACAACAACCATAAAACCAGTTACTTATAAATTGGATGGTGTTG |
| | TTTGTACAGAAATTGACCCTAAGTTGGACAATTATTATAAGAAAGACAATTCTTATTTCACAGAGCA |
| | ACCAATTGATCTTGTACCAAACCAACCATATCCAAACGCAAGCTTCGATAATTTTAAGTTTGTATGT |
| | GATAATATCAAATTTGCTGATGATTTAAACCAGTTAACTGGTTATAAGAAACCTGCTTCAAGAGAGC |
| | TTAAAGTTACATTTTTCCCTGACTTAAATGGTGATGTGGTGGCTATTGATTATAAACACTACACACC |
| | CTCTTTTAAGAAAGGAGCTAAATTGTTACATAAACCTATTGTTTGGCATGTTAACAATGCAACTAAT |
| | AAAGCCACGTATAAACCAAATACCTGGTGTATACGTTGTCTTTGGAGCACAAAACCAGTTGAAACAT |
| | CAAATTCGTTTGATGTACTGAAGTCAGAGGACGCGCAGGGAATGGATAATCTTGCCTGCGAAGATCT |
| | AAAACCAGTCTCTGAAGAAGTAGTGGAAAATCCTACCATACAGAAAGACGTTCTTGAGTGTAATGTG |
| | AAAACTACCGAAGTTGTAGGAGACATTATACTTAAACCAGCAAATAATAGTTTAAAAATTACAGAAG |
| | AGGTTGGCCACACAGATCTAATGGCTGCTTATGTAGACAATTCTAGTCTTACTATTAAGAAACCTAA |
| | TGAATTATCTAGAGTATTAGGTTTGAAAACCCTTGCTACTCATGGTTTAGCTGCTGTTAATAGTGTC |
| | CCTTGGGATACTATAGCTAATTATGCTAAGCCTTTTCTTAACAAAGTTGTTAGTACAACTACTAACA |
| | TAGTTACACGGTGTTTAAACCGTGTTTGTACTAATTATATGCCTTATTTCTTTACTTTATTGCTACA |
| | ATTGTGTACTTTTACTAGAAGTACAAATTCTAGAATTAAAGCATCTATGCCGACTACTATAGCAAAG |
| | AATACTGTTAAGAGTGTCGGTAAATTTTGTCTAGAGGCTTCATTTAATTATTTGAAGTCACCTAATT |
| | TTTCTAAACTGATAAATATTATAATTTGGTTTTTACTATTAAGTGTTTGCCTAGGTTCTTTAATCTA |
| | CTCAACCGCTGCTTTAGGTGTTTTAATGTCTAATTTAGGCATGCCTTCTTACTGTACTGGTTACAGA |
| | GAAGGCTATTTGAACTCTACTAATGTCACTATTGCAACCTACTGTACTGGTTCTATACCTTGTAGTG |
| | TTTGTCTTAGTGGTTTAGATTCTTTAGACACCTATCCTTCTTTAGAAACTATACAAATTACCATTTC |
| | ATCTTTTAAATGGGATTTAACTGCTTTTGGCTTAGTTGCAGAGTGGTTTTTGGCATATATTCTTTTC |
| | ACTAGGTTTTTCTATGTACTTGGATTGGCTGCAATCATGCAATTGTTTTTCAGCTATTTTGCAGTAC |
| | ATTTTATTAGTAATTCTTGGCTTATGTGGTTAATAATTAATCTTGTACAAATGGCCCCGATTTCAGC |
| | TATGGTTAGAATGTACATCTTCTTTGCATCATTTTATTATGTATGGAAAAGTTATGTGCATGTTGTA |
| | GACGGTTGTAATTCATCAACTTGTATGATGTGTTACAAACGTAATAGAGCAACAAGAGTCGAATGTA |
| | CAACTATTGTTAATGGTGTTAGAAGGTCCTTTTATGTCTATGCTAATGGAGGTAAAGGCTTTTGCAA |
| | ACTACACAATTGGAATTGTGTTAATTGTGATACATTCTGTGCTGGTAGTACATTTATTAGTGATGAA |
| | GTTGCGAGAGACTTGTCACTACAGTTTAAAAGACCAATAAATCCTACTGACCAGTCTTCTTACATCG |
| | TTGATAGTGTTACAGTGAAGAATGGTTCCATCCATCTTTACTTTGATAAAGCTGGTCAAAAGACTTA |
| | TGAAAGACATTCTCTCTCTCATTTTGTTAACTTAGACAACCTGAGAGCTAATAACACTAAAGGTTCA |
| | TTGCCTATTAATGTTATAGTTTTTGATGGTAAATCAAAATGTGAAGAATCATCTGCAAAATCAGCGT |
| | CTGTTTACTACAGTCAGCTTATGTGTCAACCTATACTGTTACTAGATCAGGCATTAGTGTCTGATGT |
| | TGGTGATAGTGCGGAAGTTGCAGTTAAAATGTTTGATGCTTACGTTAATACGTTTTCATCAACTTTT |
| | AACGTACCAATGGAAAAACTCAAAACACTAGTTGCAACTGCAGAAGCTGAACTTGCAAAGAATGTGT |
| | CCTTAGACAATGTCTTATCTACTTTTATTTCAGCAGCTCGGCAAGGGTTTGTTGATTCAGATGTAGA |
| | AACTAAAGATGTTGTTGAATGTCTTAAATTGTCACATCAATCTGACATAGAAGTTACTGGCGATAGT |
| | TGTAATAACTATATGCTCACCTATAACAAAGTTGAAAACATGACACCCCGTGACCTTGGTGCTTGTA |
| | TTGACTGTAGTGCGCGTCATATTAATGCGCAGGTAGCAAAAAGTCACAACATTGCTTTGATATGGAA |
| | CGTTAAAGATTTCATGTCATTGTCTGAACAACTACGAAAACAAATACGTAGTGCTGCTAAAAAGAAT |
| | AACTTACCTTTTAAGTTGACATGTGCAACTACTAGACAAGTTGTTAATGTTGTAACAACAAAGATAG |
| | CACTTAAGGGTGGTAAAATTGTTAATAATTGGTTGAAGCAGTTAATTAAAGTTACACTTGTGTTCCT |
| | TTTTGTTGCTGCTATTTTCTATTTAATAACACCTGTTCATGTCATGTCTAAACATACTGACTTTTCA |
| | AGTGAAATCATAGGATACAAGGCTATTGATGGTGGTGTCACTCGTGACATAGCATCTACAGATACTT |
| | GTTTTGCTAACAAACATGCTGATTTTGACACATGGTTTAGCCAGCGTGGTGGTAGTTATACTAATGA |
| | CAAAGCTTGCCCATTGATTGCTGCAGTCATAACAAGAGAAGTGGGTTTTGTCGTGCCTGGTTTGCCT |
| | GGCACGATATTACGCACAACTAATGGTGACTTTTTGCATTTCTTACCTAGAGTTTTTAGTGCAGTTG |
| | GTAACATCTGTTACACACCATCAAAACTTATAGAGTACACTGACTTTGCAACATCAGCTTGTGTTTT |
| | GGCTGCTGAATGTACAATTTTTAAAGATGCTTCTGGTAAGCCAGTACCATATTGTTATGATACCAAT |
| | GTACTAGAAGGTTCTGTTGCTTATGAAAGTTTACGCCCTGACACACGTTATGTGCTCATGGATGGCT |
| | CTATTATTCAATTTCCTAACACCTACCTTGAAGGTTCTGTTAGAGTGGTAACAACTTTTGATTCTGA |
| | GTACTGTAGGCACGGCACTTGTGAAAGATCAGAAGCTGGTGTTTGTGTATCTACTAGTGGTAGATGG |
| | GTACTTAACAATGATTATTACAGATCTTTACCAGGAGTTTTCTGTGGTGTAGATGCTGTAAATTTAC |
| | TTACTAATATGTTTACACCACTAATTCAACCTATTGGTGCTTTGGACATATCAGCATCTATAGTAGC |
| | TGGTGGTATTGTAGCTATCGTAGTAACATGCCTTGCCTACTATTTTATGAGGTTTAGAAGAGCTTTT |
| | GGTGAATACAGTCATGTAGTTGCCTTTAATACTTTACTATTCCTTATGTCATTCACTGTACTCTGTT |
| | TAACACCAGTTTACTCATTCTTACCTGGTGTTTATTCTGTTATTTACTTGTACTTGACATTTTATCT |
| | TACTAATGATGTTTCTTTTTTAGCACATATTCAGTGGATGGTTATGTTCACACCTTTAGTACCTTTC |
| | TGGATAACAATTGCTTATATCATTTGTATTTCCACAAAGCATTTCTATTGGTTCTTTAGTAATTACC |
| | TAAAGAGACGTGTAGTCTTTAATGGTGTTTCCTTTAGTACTTTTGAAGAAGCTGCGCTGTGCACCTT |
| | TTTGTTAAATAAAGAAATGTATCTAAAGTTGCGTAGTGATGTGCTATTACCTCTTACGCAATATAAT |
| | AGATACTTAGCTCTTTATAATAAGTACAAGTATTTTAGTGGAGCAATGGATACAACTAGCTACAGAG |
| | AAGCTGCTTGTTGTCATCTCGCAAAGGCTCTCAATGACTTCAGTAACTCAGGTTCTGATGTTCTTTA |
| | CCAACCACCACAAACCTCTATCACCTCAGCTGTTTTGCAGAGTGGTTTTAGAAAAATGGCATTCCCA |
| | TCTGGTAAAGTTGAGGGTTGTATGGTACAAGTAACTTGTGGTACAACTACACTTAACGGTCTTTGGC |
| | TTGATGACGTAGTTTACTGTCCAAGACATGTGATCTGCACCTCTGAAGACATGCTTAACCCTAATTA |
| | TGAAGATTTACTCATTCGTAAGTCTAATCATAATTTCTTGGTACAGGCTGGTAATGTTCAACTCAGG |
| | GTTATTGGACATTCTATGCAAAATTGTGTACTTAAGCTTAAGGTTGATACAGCCAATCCTAAGACAC |
| | CTAAGTATAAGTTTGTTCGCATTCAACCAGGACAGACTTTTTCAGTGTTAGCTTGTTACAATGGTTC |
| | ACCATCTGGTGTTTACCAATGTGCTATGAGGCCCAATTTCACTATTAAGGGTTCATTCCTTAATGGT |
| | TCATGTGGTAGTGTTGGTTTTAACATAGATTATGACTGTGTCTCTTTTTGTTACATGCACCATATGG |
| | AATTACCAACTGGAGTTCATGCTGGCACAGACTTAGAAGGTAACTTTTATGGACCTTTTGTTGACAG |
| | GCAAACAGCACAAGCAGCTGGTACGGACACAACTATTACAGTTAATGTTTTAGCTTGGTTGTACGCT |
| | GCTGTTATAAATGGAGACAGGTGGTTTCTCAATCGATTTACCACAACTCTTAATGACTTTAACCTTG |
| | TGGCTATGAAGTACAATTATGAACCTCTAACACAAGACCATGTTGACATACTAGGACCTCTTTCTGC |
| | TCAAACTGGAATTGCCGTTTTAGATATGTGTGCTTCATTAAAAGAATTACTGCAAAATGGTATGAAT |
| | GGACGTACCATATTGGGTAGTGCTTTATTAGAAGATGAATTTACACCTTTTGATGTTGTTAGACAAT |
| | GCTCAGGTGTTACTTTCCAAAGTGCAGTGAAAAGAACAATCAAGGGTACACACCACTGGTTGTTACT |
| | CACAATTTTGACTTCACTTTTAGTTTTAGTCCAGAGTACTCAATGGTCTTTGTTCTTTTTTTTGTAT |
| | GAAAATGCCTTTTTACCTTTTGCTATGGGTATTATTGCTATGTCTGCTTTTGCAATGATGTTTGTCA |
| | AACATAAGCATGCATTTCTCTGTTTGTTTTTGTTACCTTCTCTTGCCACTGTAGCTTATTTTAATAT |
| | GGTCTATATGCCTGCTAGTTGGGTGATGCGTATTATGACATGGTTGGATATGGTTGATACTAGTTTG |
| | TCTGGTTTTAAGCTAAAAGACTGTGTTATGTATGCATCAGCTGTAGTGTTACTAATCCTTATGACAG |
| | CAAGAACTGTGTATGATGATGGTGCTAGGAGAGTGTGGACACTTATGAATGTCTTGACACTCGTTTA |
| | TAAAGTTTATTATGGTAATGCTTTAGATCAAGCCATTTCCATGTGGGCTCTTATAATCTCTGTTACT |
| | TCTAACTACTCAGGTGTAGTTACAACTGTCATGTTTTTGGCCAGAGGTATTGTTTTTATGTGTGTTG |
| | AGTATTGCCCTATTTTCTTCATAACTGGTAATACACTTCAGTGTATAATGCTAGTTTATTGTTTCTT |
| | AGGCTATTTTTGTACTTGTTACTTTGGCCTCTTTTGTTTACTCAACCGCTACTTTAGACTGACTCTT |
| | GGTGTTTATGATTACTTAGTTTCTACACAGGAGTTTAGATATATGAATTCACAGGGACTACTCCCAC |
| | CCAAGAATAGCATAGATGCCTTCAAACTCAACATTAAATTGTTGGGTGTTGGTGGCAAACCTTGTAT |
| | CAAAGTAGCCACTGTACAGTCTAAAATGTCAGATGTAAAGTGCACATCAGTAGTCTTACTCTCAGTT |
| | TTGCAACAACTCAGAGTAGAATCATCATCTAAATTGTGGGCTCAATGTGTCCAGTTACACAATGACA |
| | TTCTCTTAGCTAAAGATACTACTGAAGCCTTTGAAAAAATGGTTTCACTACTTTCTGTTTTGCTTTC |
| | CATGCAGGGTGCTGTAGACATAAACAAGCTTTGTGAAGAAATGCTGGACAACAGGGCAACCTTACAA |
| | GCTATAGCCTCAGAGTTTAGTTCCCTTCCATCATATGCAGCTTTTGCTACTGCTCAAGAAGCTTATG |
| | AGCAGGCTGTTGCTAATGGTGATTCTGAAGTTGTTCTTAAAAAGTTGAAGAAGTCTTTGAATGTGGC |
| | TAAATCTGAATTTGACCGTGATGCAGCCATGCAACGTAAGTTGGAAAAGATGGCTGATCAAGCTATG |
| | ACCCAAATGTATAAACAGGCTAGATCTGAGGACAAGAGGGCAAAAGTTACTAGTGCTATGCAGACAA |
| | TGCTTTTCACTATGCTTAGAAAGTTGGATAATGATGCACTCAACAACATTATCAACAATGCAAGAGA |
| | TGGTTGTGTTCCCTTGAACATAATACCTCTTACAACAGCAGCCAAACTAATGGTTGTCATACCAGAC |
| | TATAACACATATAAAAATACGTGTGATGGTACAACATTTACTTATGCATCAGCATTGTGGGAAATCC |
| | AACAGGTTGTAGATGCAGATAGTAAAATTGTTCAACTTAGTGAAATTAGTATGGACAATTCACCTAA |
| | TTTAGCATGGCCTCTTATTGTAACAGCTTTAAGGGCCAATTCTGCTGTCAAATTACAGAATAATGAG |
| | CTTAGTCCTGTTGCACTACGACAGATGTCTTGTGCTGCCGGTACTACACAAACTGCTTGCACTGATG |
| | ACAATGCGTTAGCTTACTACAACACAACAAAGGGAGGTAGGTTTGTACTTGCACTGTTATCCGATTT |
| | ACAGGATTTGAAATGGGCTAGATTCCCTAAGAGTGATGGAACTGGTACTATCTATACAGAACTGGAA |
| | CCACCTTGTAGGTTTGTTACAGACACACCTAAAGGTCCTAAAGTGAAGTATTTATACTTTATTAAAG |
| | GATTAAACAACCTAAATAGAGGTATGGTACTTGGTAGTTTAGCTGCCACAGTACGTCTACAAGCTGG |
| | TAATGCAACAGAAGTGCCTGCCAATTCAACTGTATTATCTTTCTGTGCTTTTGCTGTAGATGCTGCT |
| | AAAGCTTACAAAGATTATCTAGCTAGTGGGGGACAACCAATCACTAATTGTGTTAAGATGTTGTGTA |
| | CACACACTGGTACTGGTCAGGCAATAACAGTTACACCGGAAGCCAATATGGATCAAGAATCCTTTGG |
| | TGGTGCATCGTGTTGTCTGTACTGCCGTTGCCACATAGATCATCCAAATCCTAAAGGATTTTGTGAC |
| | TTAAAAGGTAAGTATGTACAAATACCTACAACTTGTGCTAATGACCCTGTGGGTTTTACACTTAAAA |
| | ACACAGTCTGTACCGTCTGCGGTATGTGGAAAGGTTATGGCTGTAGTTGTGATCAACTCCGCGAACC |
| | CATGCTTCAGTCAGCTGATGCACAATCGTTTTTAAACGGGTTTGCGGTGTAAGTGCAGCCCGTCTTA |
| | CACCGTGCGGCACAGGCACTAGTACTGATGTCGTATACAGGGCTTTTGACATCTACAATGATAAAGT |
| | AGCTGGTTTTGCTAAATTCCTAAAAACTAATTGTTGTCGCTTCCAAGAAAAGGACGAAGATGACAAT |
| | TTAATTGATTCTTACTTTGTAGTTAAGAGACACACTTTCTCTAACTACCAACATGAAGAAACAATTT |
| | ATAATTTACTTAAGGATTGTCCAGCTGTTGCTAAACATGACTTCTTTAAGTTTAGAATAGACGGTGA |
| | CATGGTACCACATATATCACGTCAACGTCTTACTAAATACACAATGGCAGACCTCGTCTATGCTTTA |
| | AGGCATTTTGATGAAGGTAATTGTGACACATTAAAAGAAATACTTGTCACATACAATTGTTGTGATG |
| | ATGATTATTTCAATAAAAAGGACTGGTATGATTTTGTAGAAAACCCAGATATATTACGCGTATACGC |
| | CAACTTAGGTGAACGTGTACGCCAAGCTTTGTTAAAAACAGTACAATTCTGTGATGCCATGCGAAAT |
| | GCTGGTATTGTTGGTGTACTGACATTAGATAATCAAGATCTCAATGGTAACTGGTATGATTTCGGTG |
| | ATTTCATACAAACCACGCCAGGTAGTGGAGTTCCTGTTGTAGATTCTTATTATTCATTGTTAATGCC |
| | TATATTAACCTTGACCAGGGCTTTAACTGCAGAGTCACATGTTGACACTGACTTAACAAAGCCTTAC |
| | ATTAAGTGGGATTTGTTAAAATATGACTTCACGGAAGAGAGGTTAAAACTCTTTGACCGTTATTTTA |
| | AATATTGGGATCAGACATACCACCCAAATTGTGTTAACTGTTTGGATGACAGATGCATTCTGCATTG |
| | TGCAAACTTTAATGTTTTATTCTCTACAGTGTTCCCACTTACAAGTTTTGGACCACTAGTGAGAAAA |
| | ATATTTGTTGATGGTGTTCCATTTGTAGTTTCAACTGGATACCACTTCAGAGAGCTAGGTGTTGTAC |
| | ATAATCAGGATGTAAACTTACATAGCTCTAGACTTAGTTTTAAGGAATTACTTGTGTATGCTGCTGA |
| | CCCTGCTATGCACGCTGCTTCTGGTAATCTATTACTAGATAAACGCACTACGTGCTTTTCAGTAGCT |
| | GCACTTACTAACAATGTTGCTTTTCAAACTGTCAAACCCGGTAATTTTAACAAAGACTTCTATGACT |
| | TTGCTGTGTCTAAGGGTTTCTTTAAGGAAGGAAGTTCTGTTGAATTAAAACACTTCTTCTTTGCTCA |
| | GGATGGTAATGCTGCTATCAGCGATTATGACTACTATCGTTATAATCTACCAACAATGTGTGATATC |
| | AGACAACTACTATTTGTAGTTGAAGTTGTTGATAAGTACTTTGATTGTTACGATGGTGGCTGTATTA |
| | ATGCTAACCAAGTCATCGTCAACAACCTAGACAAATCAGCTGGTTTTCCATTTAATAAATGGGGTAA |
| | GGCTAGACTTTATTATGATTCAATGAGTTATGAGGATCAAGATGCACTTTTCGCATATACAAAACGT |
| | AATGTCATCCCTACTATAACTCAAATGAATCTTAAGTATGCCATTAGTGCAAAGAATAGAGCTCGCA |
| | CCGTAGCTGGTGTCTCTATCTGTAGTACTATGACCAATAGACAGTTTCATCAAAAATTATTGAAATC |
| | AATAGCCGCCACTAGAGGAGCTACTGTAGTAATTGGAACAAGCAAATTCTATGGTGGTTGGCACAAC |
| | ATGTTAAAAACTGTTTATAGTGATGTAGAAAACCCTCACCTTATGGGTTGGGATTATCCTAAATGTG |
| | ATAGAGCCATGCCTAACATGCTTAGAATTATGGCCTCACTTGTTCTTGCTCGCAAACATACAACGTG |
| | TTGTAGCTTGTCACACCGTTTCTATAGATTAGCTAATGAGTGTGCTCAAGTATTGAGTGAAATGGTC |
| | ATGTGTGGCGGTTCACTATATGTTAAACCAGGTGGAACCTCATCAGGAGATGCCACAACTGCTTATG |
| | CTAATAGTGTTTTTAACATTTGTCAAGCTGTCACGGCCAATGTTAATGCACTTTTATCTACTGATGG |
| | TAACAAAATTGCCGATAAGTATGTCCGCAATTTACAACACAGACTTTATGAGTGTCTCTATAGAAAT |
| | AGAGATGTTGACACAGACTTTGTGAATGAGTTTTACGCATATTTGCGTAAACATTTCTCAATGATGA |
| | TACTCTCTGACGATGCTGTTGTGTGTTTCAATAGCACTTATGCATCTCAAGGTCTAGTGGCTAGCAT |
| | AAAGAACTTTAAGTCAGTTCTTTATTATCAAAACAATGTTTTTATGTCTGAAGCAAAATGTTGGACT |
| | GAGACTGACCTTACTAAAGGACCTCATGAATTTTGCTCTCAACATACAATGCTAGTTAAACAGGGTG |
| | ATGATTATGTGTACCTTCCTTACCCAGATCCATCAAGAATCCTAGGGGCCGGCTGTTTTGTAGATGA |
| | TATCGTAAAAACAGATGGTACACTTATGATTGAACGGTTCGTGTCTTTAGCTATAGATGCTTACCCA |
| | CTTACTAAACATCCTAATCAGGAGTATGCTGATGTCTTTCATTTGTACTTACAATACATAAGAAAGC |
| | TACATGATGAGTTAACAGGACACATGTTAGACATGTATTCTGTTATGCTTACTAATGATAACACTTC |
| | AAGGTATTGGGAACCTGAGTTTTATGAGGCTATGTACACACCGCATACAGTCTTACAGGCTGTTGGG |
| | GCTTGTGTTCTTTGCAATTCACAGACTTCATTAAGATGTGGTGCTTGCATACGTAGACCATTCTTAT |
| | GTTGTAAATGCTGTTAGGAGCATGTCATATCAACATCACATAAATTAGTCTTGTCTGTTAATCCGTA |
| | TGTTTGCAATGCTCCAGGTTGTGATGTCACAGATGTGACTCAACTTTACTTAGGAGGTATGAGCTAT |
| | TATTGTAAATCACATAAACCACCCATTAGTTTTCCATTGTGTGCTAATGGACAAGTTTTTGGTTTAT |
| | ATAAAAATACATGTGTTGGTAGCGATAATGTTACTGACTTTAATGCAATTGCAACATGTGACTGGAC |
| | AAATGCTGGTGATTACATTTTAGCTAACACCTGTACTGAAAGACTCAAGCTTTTTGCAGCAGAAACG |
| | CTCAAAGCTACTGAGGAGACATTTAAACTGTCTTATGGTATTGCTACTGTACGTGAAGTGCTGTCTG |
| | ACAGAGAATTACATCTTTCATGGGAAGTTGGTAAACCTAGACCACCACTTAACCGAAATTATGTCTT |
| | TACTGGTTATCGTGTAACTAAAAACAGTAAAGTACAAATAGGAGAGTACACCTTTGAAAAAGGTGAC |
| | TATGGTGATGCTGTTGTTTACCGAGGTACAACAACTTACAAATTAAATGTTGGTGATTATTTTGTGC |
| | TGACATCACATACAGTAATGCCATTAAGTGCACCTACACTAGTGCCACAAGAGCACTATGTTAGAAT |
| | TACTGGCTTATACCCAACACTCAATATCTCAGATGAGTTTTCTAGCAATGTTGCAAATTATCAAAAG |
| | GTTGGTATGCAAAAGTATTCTACACTCCAGGGACCACCTGGTACTGGTAAGAGTCATTTTGCTATTG |
| | GCCTAGCTCTCTACTACCCTTCTGCTCGCATAGTGTATACAGCTTGCTCTCATGCCGCTGTTGATGC |
| | ACTATGTGAGAAGGCATTAAAATATTTGCCTATAGATAAATGTAGTAGAATTATACCTGCACGTGCT |
| | CGTGTAGAGTGTTTTGATAAATTCAAAGTGAATTCAACATTAGAACAGTATGTCTTTTGTACTGTAA |
| | ATGCATTGCCTGAGACGACAGCAGATATAGTTGTCTTTGATGAAATTTCAATGGCCACAAATTATGA |
| | TTTGAGTGTTGTCAATGCCAGATTACGTGCTAAGCACTATGTGTACATTGGCGACCCTGCTCAATTA |
| | CCTGCACCACGCACATTGCTAACTAAGGGCACACTAGAACCAGAATATTTCAATTCAGTGTGTAGAC |
| | TTATGAAAACTATAGGTCCAGACATGTTCCTCGGAACTTGTCGGCGTTGTCCTGCTGAAATTGTTGA |
| | CACTGTGAGTGCTTTGGTTTATGATAATAAGCTTAAAGCACATAAAGACAAATCAGCTCAATGCTTT |
| | AAAATGTTTTATAAGGGTGTTATCACGCATGATGTTTCATCTGCAATTAACAGGCCACAAATAGGCG |
| | TGGTAAGAGAATTCCTTACACGTAACCCTGCTTGGAGAAAAGCTGTCTTTATTTCACCTTATAATTC |
| | ACAGAATGCTGTAGCCTCAAAGATTTTGGGACTACCAACTCAAACTGTTGATTCATCACAGGGCTCA |
| | GAATATGACTATGTCATATTCACTCAAACCACTGAAACAGCTCACTCTTGTAATGTAAACAGATTTA |
| | ATGTTGCTATTACCAGAGCAAAAGTAGGCATACTTTGCATAATGTCTGATAGAGACCTTTATGACAA |
| | GTTGCAATTTACAAGTCTTGAAATTCCACGTAGGAATGTGGCAACTTTACAAGCTGAAAATGTAACA |
| | GGACTCTTTAAAGATTGTAGTAAGGTAATCACTGGGTTACATCCTACACAGGCACCTACACACCTCA |
| | GTGTTGACACTAAATTCAAAACTGAAGGTTTATGTGTTGACATACCTGGCATACCTAAGGACATGAC |
| | CTATAGAAGACTCATCTCTATGATGGGTTTTAAAATGAATTATCAAGTTAATGGTTACCCTAACATG |
| | TTTATCACCCGCGAAGAAGCTATAAGACATGTACGTGCATGGATTGGCTTCGATGTCGAGGGGTGTC |
| | ATGCTACTAGAGAAGCTGTTGGTACCAATTTACCTTTACAGCTAGGTTTTTCTACAGGTGTTAACCT |
| | AGTTGCTGTACCTACAGGTTATGTTGATACACCTAATAATACAGATTTTTCCAGAGTTAGTGCTAAA |
| | CCACCGCCTGGAGATCAATTTAAACACCTCATACCACTTATGTACAAAGGACTTCCTTGGAATGTAG |
| | TGCGTATAAAGATTGTACAAATGTTAAGTGACACACTTAAAAATCTCTCTGACAGAGTCGTATTTGT |
| | CTTATGGGCACATGGCTTTGAGTTGACATCTATGAAGTATTTTGTGAAAATAGGACCTGAGCGCACC |
| | TGTTGTCTATGTGATAGACGTGCCACATGCTTTTCCACTGCTTCAGACACTTATGCCTGTTGGCATC |
| | ATTCTATTGGATTTGATTACGTCTATAATCCGTTTATGATTGATGTTCAACAATGGGGTTTTACAGG |
| | TAACCTACAAAGCAACCATGATCTGTATTGTCAAGTCCATGGTAATGCACATGTAGCTAGTTGTGAT |
| | GCAATCATGACTAGGTGTCTAGCTGTCCACGAGTGCTTTGTTAAGCGTGTTGACTGGACTATTGAAT |
| | ATCCTATAATTGGTGATGAACTGAAGATTAATGCGGCTTGTAGAAAGGTTCAACACATGGTTGTTAA |
| | AGCTGCATTATTAGCAGACAAATTCCCAGTTCTTCACGACATTGGTAACCCTAAAGCTATTAAGTGT |
| | GTACCTCAAGCTGATGTAGAATGGAAGTTCTATGATGCACAGCCTTGTAGTGACAAAGCTTATAAAA |
| | TAGAAGAATTATTCTATTCTTATGCCACACATTCTGACAAATTCACAGATGGTGTATGCCTATTTTG |
| | GAATTGCAATGTCGATAGATATCCTGCTAATTCCATTGTTTGTAGATTTGACACTAGAGTGCTATCT |
| | AACCTTAACTTGCCTGGTTGTGATGGTGGCAGTTTGTATGTAAATAAACATGCATTCCACACACCAG |
| | CTTTTGATAAAAGTGCTTTTGTTAATTTAAAACAATTACCATTTTTCTATTACTCTGACAGTCCATG |
| | TGAGTCTCATGGAAAACAAGTAGTGTCAGATATAGATTATGTACCACTAAAGTCTGCTACGTGTATA |
| | ACACGTTGCAATTTAGGTGGTGCTGTCTGTAGACATCATGCTAATGAGTACAGATTGTATCTCGATG |
| | CTTATAACATGATGATCTCAGCTGGCTTTAGCTTGTGGGTTTACAAACAATTTGATACTTATAACCT |
| | CTGGAACACTTTTACAAGACTTCAGAGTTTAGAAAATGTGGCTTTTAATGTTGTAAATAAGGGACAC |
| | TTTGATGGACAACAGGGTGAAGTACCAGTTTCTATCATTAATAACACTGTTTACACAAAAGTTGATG |
| | GTGTTGATGTAGAATTGTTTGAAAATAAAACAACATTACCTGTTAATGTAGCATTTGAGCTTTGGGC |
| | TAAGCGCAACATTAAACCAGTACCAGAGGTGAAAATACTCAATAATTTGGGTGTGGACATTGCTGCT |
| | AATACTGTGATCTGGGACTACAAAAGAGATGCTCCAGCACATATATCTACTATTGGTGTTTGTTCTA |
| | TGACTGACATAGCCAAGAAACCAACTGAAACGATTTGTGCACCACTCACTGTCTTTTTTGATGGTAG |
| | AGTTGATGGTCAAGTAGACTTATTTAGAAATGCCCGTAATGGTGTTCTTATTACAGAAGGTAGTGTT |
| | AAAGGTTTACAACCATCTGTAGGTCCCAAACAAGCTAGTCTTAATGGAGTCACATTAATTGGAGAAG |
| | CCGTAAAAACACAGTTCAATTATTATAAGAAAGTTGATGGTGTTGTCCAACAATTACCTGAAACTTA |
| | CTTTACTCAGAGTAGAAATTTACAAGAATTTAAACCCAGGAGTCAAATGGAAATTGATTTCTTAGAA |
| | TTAGCTATGGATGAATTCATTGAACGGTATAAATTAGAAGGCTATGCCTTCGAACATATCGTTTATG |
| | GAGATTTTAGTCATAGTCAGTTAGGTGGTTTACATCTACTGATTGGACTAGCTAAACGTTTTAAGGA |
| | ATCACCTTTTGAATTAGAAGATTTTATTCCTATGGACAGTACAGTTAAAAACTATTTCATAACAGAT |
| | GCGCAAACAGGTTCATCTAAGTGTGTGTGTTCTGTTATTGATTTATTACTTGATGATTTTGTTGAAA |
| | TAATAAAATCCCAAGATTTATCTGTAGTTTCTAAGGTTGTCAAAGTGACTATTGACTATACAGAAAT |
| | TTCATTTATGCTTTGGTGTAAAGATGGCCATGTAGAAACATTTTACCCAAAATTACAATCTAGTCAA |
| | GCGTGGCAACCGGGTGTTGCTATGCCTAATCTTTACAAAATGCAAAGAATGCTATTAGAAAAGTGTG |
| | ACCTTCAAAATTATGGTGATAGTGCAACATTACCTAAAGGCATAATGATGAATGTCGCAAAATATAC |
| | TCAACTGTGTCAATATTTAAACACATTAACATTAGCTGTACCCTATAATATGAGAGTTATACATTTT |
| | GGTGCTGGTTCTGATAAAGGAGTTGCACCAGGTACAGCTGTTTTAAGACAGTGGTTGCCTACGGGTA |
| | CGCTGCTTGTCGATTCAGATCTTAATGACTTTGTCTCTGATGCAGATTCAACTTTGATTGGTGATTG |
| | TGCAACTGTACATACAGCTAATAAATGGGATCTCATTATTAGTGATATGTACGACCCTAAGACTAAA |
| | AATGTTACAAAAGAAAATGACTCTAAAGAGGGTTTTTTCACTTACATTTGTGGGTTTATACAACAAA |
| | AGCTAGCTCTTGGAGGTTCCGTGGCTATAAAGATAACAGAACATTCTTGGAATGCTGATCTTTATAA |
| | GCTCATGGGACACTTCGCATGGTGGACAGCCTTTGTTACTAATGTGAATGCGTCATCATCTGAAGCA |
| | TTTTTAATTGGATGTAATTATCTTGGCAAACCACGCGAACAAATAGATGGTTATGTCATGCATGCAA |
| | ATTACATATTTTGGAGGAATACAAATCCAATTCAGTTGTCTTCCTATTCTTTATTTGACATGAGTAA |
| | ATTTCCCCTTAAATTAAGGGGTACTGCTGTTATGTCTTTAAAAGAAGGTCAAATCAATGATATGATT |
| | TTATCTCTTCTTAGTAAAGGTAGACTTATAATTAGAGAAAACAACAGAGTTGTTATTTCTAGTGATG |
| | TTCTTGTTAACAACTAAACGAACAATGTTTGTTTTTCTTGTTTTATTGCCACTAGTCTCTAGTCAGT |
| | GTGTTAATCTTACAACCAGAACTCAATTACCCCCTGCATACACTAATTCTTTCACACGTGGTGTTTA |
| | TTACCCTGACAAAGTTTTCAGATCCTCAGTTTTACATTCAACTCAGGACTTGTTCTTACCTTTCTTT |
| | TCCAATGTTACTTGGTTCCATGCTATACATGTCTCTGGGACCAATGGTACTAAGAGGTTTGATAACC |
| | CTGTCCTACCATTTAATGATGGTGTTTATTTTGCTTCCACTGAGAAGTCTAACATAATAAGAGGCTG |
| | GATTTTTGGTACTACTTTAGATTCGAAGACCCAGTCCCTACTTATTGTTAATAACGCTACTAATGTT |
| | GTTATTAAAGTCTGTGAATTTCAATTTTGTAATGATCCATTTTTGGGTGTTTATTACCACAAAAACA |
| | ACAAAAGTTGGATGGAAAGTGAGTTCAGAGTTTATTCTAGTGCGAATAATTGCACTTTTGAATATGT |
| | CTCTCAGCCTTTTCTTATGGACCTTGAAGGAAAACAGGGTAATTTCAAAAATCTTAGGGAATTTGTG |
| | TTTAAGAATATTGATGGTTATTTTAAAATATATTCTAAGCACACGCCTATTAATTTAGTGCGTGATC |
| | TCCCTCAGGGTTTTTCGGCTTTAGAACCATTGGTAGATTTGCCAATAGGTATTAACATCACTAGGTT |
| | TCAAACTTTACTTGCTTTACATAGAAGTTATTTGACTCCTGGTGATTCTTCTTCAGGTTGGACAGCT |
| | GGTGCTGCAGCTTATTATGTGGGTTATCTTCAACCTAGGACTTTTCTATTAAAATATAATGAAAATG |
| | GAACCATTACAGATGCTGTAGACTGTGCACTTGACCCTCTCTCAGAAACAAAGTGTACGTTGAAATC |
| | CTTCACTGTAGAAAAAGGAATCTATCAAACTTCTAACTTTAGAGTCCAACCAACAGAATCTATTGTT |
| | AGATTTCCTAATATTACAAACTTGTGCCCTTTTGGTGAAGTTTTTAACGCCACCAGATTTGCATCTG |
| | TTTATGCTTGGAACAGGAAGAGAATCAGCAACTGTGTTGCTGATTATTCTGTCCTATATAATTCCGC |
| | ATCATTTTCCACTTTTAAGTGTTATGGAGTGTCTCCTACTAAATTAAATGATCTCTGCTTTACTAAT |
| | GTCTATGCAGATTCATTTGTAATTAGAGGTGATGAAGTCAGACAAATCGCTCCAGGGCAAACTGGAA |
| | AGATTGCTGATTATAATTATAAATTACCAGATGATTTTACAGGCTGCGTTATAGCTTGGAATTCTAA |
| | CAATCTTGATTCTAAGGTTGGTGGTAATTATAATTACCTGTATAGATTGTTTAGGAAGTCTAATCTC |
| | AAACCTTTTGAGAGAGATATTTCAACTGAAATCTATCAGGCCGGTAGCACACCTTGTAATGGTGTTG |
| | AAGGTTTTAATTGTTACTTTCCTTTACAATCATATGGTTTCCAACCCACTAATGGTGTTGGTTACCA |
| | ACCATACAGAGTAGTAGTACTTTCTTTTGAACTTCTACATGCACCAGCAACTGTTTGTGGACCTAAA |
| | AAGTCTACTAATTTGGTTAAAAACAAATGTGTCAATTTCAACTTCAATGGTTTAACAGGCACAGGTG |
| | TTCTTACTGAGTCTAACAAAAAGTTTCTGCCTTTCCAACAATTTGGCAGAGACATTGCTGACACTAC |
| | TGATGCTGTCCGTGATCCACAGACACTTGAGATTCTTGACATTACACCATGTTCTTTTGGTGGTGTC |
| | AGTGTTATAACACCAGGAACAAATACTTCTAACCAGGTTGCTGTTCTTTATCAGGGTGTTAACTGCA |
| | CAGAAGTCCCTGTTGCTATTCATGCAGATCAACTTACTCCTACTTGGCGTGTTTATTCTACAGGTTC |
| | TAATGTTTTTCAAACACGTGCAGGCTGTTTAATAGGGGCTGAACATGTCAACAACTCATATGAGTGT |
| | GACATACCCATTGGTGCAGGTATATGCGCTAGTTATCAGACTCAGACTAATTCTCCTCGGCGGGCAC |
| | GTAGTGTAGCTAGTCAATCCATCATTGCCTACACTATGTCACTTGGTGCAGAAAATTCAGTTGCTTA |
| | CTCTAATAACTCTATTGCCATACCCACAAATTTTACTATTAGTGTTACCACAGAAATTCTACCAGTG |
| | TCTATGACCAAGACATCAGTAGATTGTACAATGTACATTTGTGGTGATTCAACTGAATGCAGCAATC |
| | TTTTGTTGCAATATGGCAGTTTTTGTACACAATTAAACCGTGCTTTAACTGGAATAGCTGTTGAACA |
| | AGACAAAAACACCCAAGAAGTTTTTGCACAAGTCAAACAAATTTACAAAACACCACCAATTAAAGAT |
| | TTTGGTGGTTTTAATTTTTCACAAATATTAGCAGATCCATCAAAACCAAGCAAGAGGTCATTTATTG |
| | AAGATCTACTTTTCAACAAAGTGACACTTGCAGATGCTGGCTTCATCAAACAATATGGTGATTGCCT |
| | TGGTGATATTGCTGCTAGAGACCTCATTTGTGCACAAAAGTTTAACGGCCTTACTGTTTTGCCACCT |
| | TTGCTCACAGATGAAATGATTGCTCAATACACTTCTGCACTGTTAGCGGGTACAATCACTTCTGGTT |
| | GGACCTTTGGTGCAGGTGCTGCATTACAAATACCATTTGCTATGCAAATGGCTTATAGGTTTAATGG |
| | TATTGGAGTTACACAGAATGTTCTCTATGAGAACCAAAAATTGATTGCCAACCAATTTAATAGTGCT |
| | ATTGGCAAAATTCAAGACTCACTTTCTTCCACAGCAAGTGCACTTGGAAAACTTCAAGATGTGGTCA |
| | ACCAAAATGCACAAGCTTTAAACACGCTTGTTAAACAACTTAGCTCCAATTTTGGTGCAATTTCAAG |
| | TGTTTTAAATGATATCCTTTCACGTCTTGACAAAGTTGAGGCTGAAGTGCAAATTGATAGGTTGATC |
| | ACAGGCAGACTTCAAAGTTTGCAGACATATGTGACTCAACAATTAATTAGAGCTGCAGAAATCAGAG |
| | CTTCTGCTAATCTTGCTGCTACTAAAATGTCAGAGTGTGTACTTGGACAATCAAAAAGAGTTGATTT |
| | TTGTGGAAAGGGCTATCATCTTATGTCCTTCCCTCAGTCAGCACCTCATGGTGTAGTCTTCTTGCAT |
| | GTGACTTATGTCCCTGCACAAGAAAAGAACTTCACAACTGCTCCTGCCATTTGTCATGATGGAAAAG |
| | CACACTTTCCTCGTGAAGGTGTCTTTGTTTCAAATGGCACACACTGGTTTGTAACACAAAGGAATTT |
| | TTATGAACCACAAATCATTACTACAGACAACACATTTGTGTCTGGTAACTGTGATGTTGTAATAGGA |
| | ATTGTCAACAACACAGTTTATGATCCTTTGCAACCTGAATTAGACTCATTCAAGGAGGAGTTAGATA |
| | AATATTTTAAGAATCATACATCACCAGATGTTGATTTAGGTGACATCTCTGGCATTAATGCTTCAGT |
| | TGTAAACATTCAAAAAGAAATTGACCGCCTCAATGAGGTTGCCAAGAATTTAAATGAATCTCTCATC |
| | GATCTCCAAGAACTTGGAAAGTATGAGCAGTATATAAAATGGCCATGGTACATTTGGCTAGGTTTTA |
| | TAGCTGGCTTGATTGCCATAGTAATGGTGACAATTATGCTTTGCTGTATGACCAGTTGCTGTAGTTG |
| | TCTCAAGGGCTGTTGTTCTTGTGGATCCTGCTGCAAATTTGATGAAGACGACTCTGAGCCAGTGCTC |
| | AAAGGAGTCAAATTACATTACACATAAACGAACTTATGGATTTGTTTATGAGAATCTTCACAATTGG |
| | AACTGTAACTTTGAAGCAAGGTGAAATCAAGGATGCTACTCCTTCAGATTTTGTTCGCGCTACTGCA |
| | ACGATACCGATACAAGCCTCACTCCCTTTCGGATGGCTTATTGTTGGCGTTGCACTTCTTGCTGTTT |
| | TTCAGAGCGCTTCCAAAATCATAACCCTCAAAAAGAGATGGCAACTAGCACTCTCCAAGGGTGTTCA |
| | CTTTGTTTGCAACTTGCTGTTGTTGTTTGTAACAGTTTACTCACACCTTTTGCTCGTTGCTGCTGGC |
| | CTTGAAGCCCCTTTTCTCTATCTTTATGCTTTAGTCTACTTCTTGCAGAGTATAAACTTTGTAAGAA |
| | TAATAATGAGGCTTTGGCTTTGCTGGAAATGCCGTTCCAAAAACCCATTACTTTATGATGCCAACTA |
| | TTTTCTTTGCTGGCATACTAATTGTTACGACTATTGTATACCTTACAATAGTGTAACTTCTTCAATT |
| | GTCATTACTTCAGGTGATGGCACAACAAGTCCTATTTCTGAACATGACTACCAGATTGGTGGTTATA |
| | CTGAAAAATGGGAATCTGGAGTAAAAGACTGTGTTGTATTACACAGTTACTTCACTTCAGACTATTA |
| | CCAGCTGTACTCAACTCAATTGAGTACAGACACTGGTGTTGAACATGTTACCTTCTTCATCTACAAT |
| | AAAATTGTTGATGAGCCTGAAGAACATGTCCAAATTCACACAATCGACGGTTCATCCGGAGTTGTTA |
| | ATCCAGTAATGGAACCAATTTATGATGAACCGACGACGACTACTAGCGTGCCTTTGTAAGCACAAGC |
| | TGATGAGTACGAACTTATGTACTCATTCGTTTCGGAAGAGACAGGTACGTTAATAGTTAATAGCGTA |
| | CTTCTTTTTCTTGCTTTCGTGGTATTCTTGCTAGTTACACTAGCCATCCTTACTGCGCTTCGATTGT |
| | GTGCGTACTGCTGCAATATTGTTAACGTGAGTCTTGTAAAACCTTCTTTTTACGTTTACTCTCGTGT |
| | TAAAAATCTGAATTCTTCTAGAGTTCCTGATCTTCTGGTCTAAACGAACTAAATATTATATTAGTTT |
| | TTCTGTTTGGAACTTTAATTTTAGCCATGGCAGATTCCAACGGTACTATTACCGTTGAAGAGCTTAA |
| | AAAGCTCCTTGAACAATGGAACCTAGTAATAGGTTTCCTATTCCTTACATGGATTTGTCTTCTACAA |
| | TTTGCCTATGCCAACAGGAATAGGTTTTTGTATATAATTAAGTTAATTTTCCTCTGGCTGTTATGGC |
| | CAGTAACTTTAGCTTGTTTTGTGCTTGCTGCTGTTTACAGAATAAATTGGATCACCGGTGGAATTGC |
| | TATCGCAATGGCTTGTCTTGTAGGCTTGATGTGGCTCAGCTACTTCATTGCTTCTTTCAGACTGTTT |
| | GCGCGTACGCGTTCCATGTGGTCATTCAATCCAGAAACTAACATTCTTCTCAACGTGCCACTCCATG |
| | GCACTATTCTGACCAGACCGCTTCTAGAAAGTGAACTCGTAATCGGAGCTGTGATCCTTCGTGGACA |
| | TCTTCGTATTGCTGGACACCATCTAGGACGCTGTGACATCAAGGACCTGCCTAAAGAAATCACTGTT |
| | GCTACATCACGAACGCTTTCTTATTACAAATTGGGAGCTTCGCAGCGTGTAGCAGGTGACTCAGGTT |
| | TTGCTGCATACAGTCGCTACAGGATTGGCAACTATAAATTAAACACAGACCATTCCAGTAGCAGTGA |
| | CAATATTGCTTTGCTTGTACAGTAAGTGACAACAGATGTTTCATCTCGTTGACTTTCAGGTTACTAT |
| | AGCAGAGATATTACTAATTATTATGAGGACTTTTAAAGTTTCCATTTGGAATCTTGATTACATCATA |
| | AACCTCATAATTAAAAATTTATCTAAGTCACTAACTGAGAATAAATATTCTCAATTAGATGAAGAGC |
| | AACCAATGGAGATTGATTAAACGAACATGAAAATTATTCTTTTCTTGGCACTGATAACACTCGCTAC |
| | TTGTGAGCTTTATCACTACCAAGAGTGTGTTAGAGGTACAACAGTACTTTTAAAAGAACCTTGCTCT |
| | TCTGGAACATACGAGGGCAATTCACCATTTCATCCTCTAGCTGATAACAAATTTGCACTGACTTGCT |
| | TTAGCACTCAATTTGCTTTTGCTTGTCCTGACGGCGTAAAACACGTCTATCAGTTACGTGCCAGATC |
| | AGTTTCACCTAAACTGTTCATCAGACAAGAGGAAGTTCAAGAACTTTACTCTCCAATTTTTCTTATT |
| | GTTGCGGCAATAGTGTTTATAACACTTTGCTTCACACTCAAAAGAAAGACAGAATGATTGAACTTTC |
| | ATTAATTGACTTCTATTTGTGCTTTTTAGCCTTTCTGCTATTCCTTGTTTTAATTATGCTTATTATC |
| | TTTTGGTTCTCACTTGAACTGCAAGATCATAATGAAACTTGTCACGCCTAAACGAACATGAAATTTC |
| | TTGTTTTCTTAGGAATCATCACAACTGTAGCTGCATTTCACCAAGAATGTAGTTTACAGTCATGTAC |
| | TCAACATCAACCATATGTAGTTGATGACCCGTGTCCTATTCACTTCTATTCTAAATGGTATATTAGA |
| | GTAGGAGCTAGAAAATCAGCACCTTTAATTGAATTGTGCGTGGATGAGGCTGGTTCTAAATCACCCA |
| | TTCAGTACATCGATATCGGTAATTATACAGTTTCCTGTTTACCTTTTACAATTAATTGCCAGGAACC |
| | TAAATTGGGTAGTCTTGTAGTGCGTTGTTCGTTCTATGAAGACTTTTTAGAGTATCATGACGTTCGT |
| | GTTGTTTTAGATTTCATCTAAACGAACAAACTAAAATGTCTGATAATGGACCCCAAAATCAGCGAAA |
| | TGCACCCCGCATTACGTTTGGTGGACCCTCAGATTCAACTGGCAGTAACCAGAATGGAGAACGCAGT |
| | GGGGCGCGATCAAAACAACGTCGGCCCCAAGGTTTACCCAATAATACTGCGTCTTGGTTCACCGCTC |
| | TCACTCAACATGGCAAGGAAGACCTTAAATTCCCTCGAGGACAAGGCGTTCCAATTAACACCAATAG |
| | CAGTCCAGATGACCAAATTGGCTACTACCGAAGAGCTACCAGACGAATTCGTGGTGGTGACGGTAAA |
| | ATGAAAGATCTCAGTCCAAGATGGTATTTCTACTACCTAGGAACTGGGCCAGAAGCTGGACTTCCCT |
| | ATGGTGCTAACAAAGACGGCATCATATGGGTTGCAACTGAGGGAGCCTTGAATACACCAAAAGATCA |
| | CATTGGCACCCGCAATCCTGCTAACAATGCTGCAATCGTGCTACAACTTCCTCAAGGAACAACATTG |
| | CCAAAAGGCTTCTACGCAGAAGGGAGCAGAGGCGGCAGTCAAGCCTCTTCTCGTTCCTCATCACGTA |
| | GTCGCAACAGTTCAAGAAATTCAACTCCAGGCAGCAGTAAACGAACTTCTCCTGCTAGAATGGCTGG |
| | CAATGGCGGTGATGCTGCTCTTGCTTTGCTGCTGCTTGACAGATTGAACCAGCTTGAGAGCAAAATG |
| | TCTGGTAAAGGCCAACAACAACAAGGCCAAACTGTCACTAAGAAATCTGCTGCTGAGGCTTCTAAGA |
| | AGCCTCGGCAAAAACGTACTGCCACTAAAGCATACAATGTAACACAAGCTTTCGGCAGACGTGGTCC |
| | AGAACAAACCCAAGGAAATTTTGGGGACCAGGAACTAATCAGACAAGGAACTGATTACAAACATTGG |
| | CCGCAAATTGCACAATTTGCCCCCAGCGCTTCAGCGTTCTTCGGAATGTCGCGCATTGGCATGGAAG |
| | TCACACCTTCGGGAACGTGGTTGACCTACACAGGTGCCATCAAATTGGATGACAAAGATCCAAATTT |
| | CAAAGATCAAGTCATTTTGCTGAATAAGCATATTGACGCATACAAAACATTCCCACCAACAGAGCCT |
| | AAAAAGGACAAAAAGAAGAAGGCTGATGAAACTCAAGCCTTACCGCAGAGACAGAAGAAACAGCAAA |
| | CTGTGACTCTTCTTCCTGCTGCAGATTTGGATGATTTCTCCAAACAATTGCAACAATCCATGAGCAG |
| | TGCTGACTCAACTCAGGCCTAAACTCATGCAGACCACACAAGGCAGATGGGCTATATAAACGTTTTC |
| | GCTTTTCCGTTTACGATATATAGTCTACTCTTGTGCAGAATGAATTCTCGTAACTACATAGCACAAG |
| | TAGATGTAGTTAACTTTAATCTCACATAGCAATCTTTAATCAGTGTGTAACATTAGGGAGGACTTGA |
| | AAGAGCCACCACATTTTCACCGAGGCCACGCGGAGTACGATCGAGTGTACAGTGAACAATGCTAGGG |
| | AGAGCTGCCTATATGGAAGAGCCCTAATGTGTAAAATTAATTTTAGTAGTGCTATCCCCATGTGATT |
| | TTAATAGCTTCTTAGGAGAATGACAAAAA |
| 6 | GenBank Accession No. NC_006577.2 (Human coronavirus HKU1, complete |
| | genome) |
| | GAGTTTGAGCGATTGACGTTCGTACCGTCTATCAGCTTACGATCTCTTGTCAGATCTCATTAAATCT |
| | AAACTTTTTAAACAAGATTCCCTGTTATCCATGCTTGTGAGTGTGGTTTAATCATAATCTTGTATTT |
| | TACTTTCCACACTTTTCATCTCTCTGCCAGTGACGTGTTGGTTGTCCTCAGCGTCCCTCCCATAGGT |
| | CGCAATGATTAAAACCAGCAAATACGGTCTCGGCTTCAAGTGGGCGCCAGAATTTCGTTGGCTGCTT |
| | CCGGATGCAGCGGAGGAGTTGGCTAGTCCTATGAAGTCAGATGAGGGTGGGTTATGCCCCTCTACTG |
| | GTCAAGCGATGGAAAGTGTTGGATTCGTTTATGATAATCATGTGAAGATAGATTGTCGCTGCATTCT |
| | TGGACAAGAATGGCATGTGCAGTCAAATCTTATCCGTGATATTTTTGTTCATGAAGATCTACATGTT |
| | GTAGAAGTTCTAACTAAAACAGCCGTAAAGTCCGGTACGGCAATTTTAATTAAATCACCTTTGCATA |
| | GCTTGGGTGGTTTTCCTAAAGGGTATGTTATGGGCTTGTTCCGTTCATACAAGACTAAACGTTATGT |
| | TGTACATCATCTTTCTATGACTACATCTACTACTAATTTTGGTGAAGATTTTTTGGGTTGGATTGTA |
| | CCTTTTGGTTTTATGCCATCTTATGTTCACAAATGGTTTCAATTCTGTAGGTTGTATATTGAAGAGA |
| | GTGATTTAATAATTTCAAATTTTAAATTTGATGATTATGATTTTAGTGTAGAAGATGCTTATGCTGA |
| | GGTTCATGCTGAGCCTAAAGGTAAATATTCACAAAAAGCTTATGCTTTACTTAGACAATATCGTGGT |
| | ATTAAACCCGTACTTTTTGTAGACCAGTATGGTTGTGACTATTCTGGTAAATTAGCAGATTGTCTTC |
| | AAGCTTATGGTCATTATTCTTTGCAAGATATGAGACAAAAGCAGTCTGTATGGCTTGCCAATTGTGA |
| | CTTTGATATTGTAGTGGCTTGGCATGTAGTTCGTGATTCACGATTTGTTATGCGCCTGCAGACTATA |
| | GCTACTATTTGTGGTATTAAATATGTTGCACAACCTACAGAAGATGTAGTAGATGGAGATGTAGTTA |
| | TACGTGAACCTGTACATTTATTATCTGCTGATGCAATAGTTTTAAAGCTTCCTAGTTTGATGAAAGT |
| | TATGACTCATATGGATGATTTTTCTATTAAATCTATATATAATGTTGATTTGTGTGATTGTGGTTTT |
| | GTTATGCAGTATGGTTATGTAGATTGTTTTAATGATAATTGTGATTTTTATGGTTGGGTTTCAGGTA |
| | ATATGATGGATGGTTTTTCTTGTCCATTGTGTTGTACAGTTTATGACTCTAGCGAAGTTAAAGCCCA |
| | ATCATCTGGTGTTATTCCTGAAAATCCTGTGTTATTTACTAATAGTACTGATACTGTTAACCATGAT |
| | TCTTTTAATTTGTATGGTTATTCTGTCACACCATTTGGTTCTTGTATATATTGGTCGCCGCGTCCTG |
| | GATTGTGGATTCCTATAATTAAATCTTCAGTCAAGTCTTATGATGATTTGGTTTATTCAGGTGTAGT |
| | AGGTTGTAAATCTATTGTTAAAGAAACTGCTCTTATTACTCATGCACTTTACTTAGATTATGTTCAA |
| | TGTAAGTGTGGTAATCTTGAACAAAATCATATTCTTGGCGTTAATAATTCTTGGTGTAGGCAACTGT |
| | TGCTTAATAGAGGTGATTATAATATGCTTCTAAAAAATATTGACTTGTTTGTTAAGCGTCGTGCTGA |
| | TTTTGCTTGCAAGTTTGCAGTTTGTGGAGATGGTTTTGTACCTTTTTTACTAGATGGTTTAATTCCC |
| | CGTAGTTATTATCTAATTCAGAGTGGTATTTTCTTTACATCTTTGATGTCTCAATTTTCACAAGAAG |
| | TTTCTGATATGTGTTTAAAAATGTGTATTTTGTTTATGGACAGAGTTTCAGTTGCTACATTTTATAT |
| | AGAGCATTATGTTAATAGGTTGGTTACTCAATTTAAGTTATTGGGTACTACACTTGTTAATAAAATG |
| | GTTAATTGGTTTAATACCATGTTAGATGCTAGTGCACCTGCTACAGGCTGGCTTCTTTACCAATTAT |
| | TGAATGGTCTTTTTGTAGTATCTCAAGCCAACTTTAATTTTGTTGCTTTAATACCTGATTATGCTAA |
| | AATTTTAGTTAATAAATTTTACACTTTTTTTAAGTTATTATTAGAGTGTGTTACAGTTGATGTTTTA |
| | AAAGATATGCCTGTTCTTAAAACTATTAATGGTTTAGTTTGTATTGTAGGCAATAAGTTTTATAACG |
| | TTAGTACAGGGTTAATTCCTGGTTTTGTTTTACCATGTAATGCACAGGAACAACAAATTTATTTTTT |
| | TGAAGGCGTTGCAGAATCTGTTATAGTAGAAGATGATGTTATTGAGAATGTCAAATCTTCTTTATCA |
| | TCTTATGAGTATTGTCAACCACCTAAATCTGTAGAAAAAATTTGTATTATAGATAATATGTACATGG |
| | GTAAGTGTGGTGATAAATTTTTCCCTATTGTCATGAATGATAAAAATATTTGTCTTTTAGATCAGGC |
| | TTGGCGTTTTCCATGTGCAGGTAGAAAAGTTAATTTTAACGAGAAACCTGTTGTTATGGAGATTCCG |
| | TCTTTGATGACAGTTAAGGTTATGTTTGATTTAGATTCTACTTTTGATGATATTTTAGGTAAAGTTT |
| | GTTCAGAATTTGAAGTAGAAAAGGGTGTTACTGTAGATGATTTTGTTGCTGTTGTTTGTGATGCTAT |
| | AGAGAATGCTTTAAACTCTTGTAAAGAGCATCCAGTGGTTGGTTATCAAGTTCGTGCATTTTTAAAT |
| | AAACTTAATGAGAATGTTGTTTATTTATTTGATGAGGCTGGTGATGAAGCAATGGCCTCTCGTATGT |
| | ATTGTACTTTTGCTATTGAGGATGTTGAAGACGTTATCAGTAGTGAAGCTGTCGAAGATACTATTGA |
| | TGGTGTCGTTGAAGACACTATTAATGACGATGAAGATGTTGTTACTGGTGACAATGACGATGAAGAT |
| | GTTGTTACTGGTGACAATGACGATGAAGATGTTGTTACTGGTGACAATGACGATGAAGATGTTGTTA |
| | CTGGTGACAATGACGATGAAGATGTTGTTACTGGTGACAATGACGATGAAGATGTTGTTACTGGTGA |
| | CAATGACGATGAAGATGTTGTTACTGGTGACAATGACGATGAAGATGTTGTTACTGGTGACAATGAC |
| | GATGAAGATGTTGTTACTGGTGACAATGACGATGAAGATGTTGTTACTGGTGACAATGACGATGAAG |
| | ATGTTGTTACTGGTGACAATGACGATGAAGATGTTGTTACTGGTGACAATGACGATGAAGATGTTGT |
| | TACTGGTGACAATGACGATGAAGATGTTGTTACTGGTGACAATAACGATGAAGAGATTGTTACTGGT |
| | GACAATGATGACCAAATTGTTGTTACTGGTGATGATGTAGATGATATTGAAAGTATTTATGACTTTG |
| | ATACTTATAAAGCTCTTTTAGTTTTTAATGATGTCTATAATGATGCTTTGTTTGTTAGTTATGGTTC |
| | TAGTGTTGAAACAGAAACATATTTTAAAGTTAATGGTTTATGGTCACCTACTATTACACATACTAAT |
| | TGTTGGTTGCGTTCTGTGTTACTTGTAATGCAGAAATTACCTTTTAAGTTTAAGGATTTAGCTATTG |
| | AAAATATGTGGTTATCTTATAAGGTGGGTTATAATCAAAGTTTTGTTGATTATTTACTGACCACTAT |
| | TCCTAAAGCTATTGTTTTGCCTCAAGGTGGTTTTGTAGCTGATTTTGCTTATTGGTTTTTAAACCAG |
| | TTTGATATTAATGCGTATGCTAATTGGTGTTGTTTAAAATGTGGTTTTTCTTTTGATTTAAATGGTT |
| | TGGATGCTTTGTTTTTTTATGGAGATATTGTGTCTCATGTTTGTAAGTGTGGACATAATATGACTCT |
| | AATAGCAGCGGACTTACCTTGTACATTACATTTTTCATTATTTGATGACAATTTTTGTGCTTTTTGC |
| | ACCCCTAAAAAAATTTTTATTGCTGCATGTGCTGTGGATGTAAACGTTTGTCATTCTGTAGCTGTTA |
| | TAGGTGATGAACAAATAGATGGTAAGTTTGTTACTAAATTTAGTGGTGATAAATTTGATTTTATAGT |
| | AGGTTATGGAATGTCATTTAGTATGTCTTCTTTTGAGTTACCTCAATTGTATGGTTTGTGTATAACA |
| | CCTAATGTATGTTTTGTTAAAGGTGATATTATAAATGTTGCTAGACTTGTTAAAGCTGATGTTATTG |
| | TTAATCCTGCTAATGGGCATATGCTCCATGGTGGTGGAGTTGCAAAAGCTATAGCTGTAGCTGCAGG |
| | TAAAAAATTTTCTAAAGAAACTGCTGCTATGGTTAAATCTAAAGGTGTTTGCCAAGTAGGAGATTGT |
| | TATGTTTCTACCGGTGGTAAATTATGTAAAACAATTCTTAATATTGTAGGCCCTGATGCTAGACAAG |
| | ATGGAAGACAATCTTATGTTTTGTTAGCACGTGCTTATAAGCATCTTAATAATTATGATTGTTGTTT |
| | GTCTACTCTCATATCGGCTGGTATATTTAGTGTTCCTGCTGATGTGTCATTAACTTACCTTCTAGGT |
| | GTTGTTGATAAACAAGTTATCCTTGTTAGTAATAATAAAGAAGATTTTGATATTATTCAAAAATGTC |
| | AAATTACTTCAGTTGTTGGTACTAAAGCATTGGCTGTTAGATTAACTGCTAATGTAGGCCGTGTTAT |
| | TAAATTTGAGACAGATGCATACAAACTTTTTTTGAGTGGTGATGATTGTTTTGTTTCAAATTCTTCT |
| | GTTATACAAGAAGTTTTATTGCTTCGTCATGATATACAATTGAATAATGACGTTCGTGATTATTTGT |
| | TGTCTAAGATGACTAGTCTTCCTAAAGATTGGCGTCTTATCAATAAATTTGATGTTATTAACGGTGT |
| | TAAAACTGTTAAGTATTTTGAGTGTCCTAATTCTATTTATATATGTAGTCAGGGTAAAGACTTTGGT |
| | TATGTATGTGATGGTTCTTTTTATAAAGCAACTGTTAATCAAGTTTGTGTTTTATTAGCTAAGAAGA |
| | TAGATGTTTTGCTTACTGTAGATGGTGTTAATTTTAAATCTATTTCTCTTACTGTAGGTGAAGTTTT |
| | TGGTAAAATACTTGGTAATGTTTTCTGTGATGGCATTGATGTTACTAAGTTAAAGTGTAGTGATTTT |
| | TATGCCGATAAAATTTTATATCAGTATGAAAATTTGTCTTTAGCTGATATTTCTGCTGTACAAAGTT |
| | CATTTGGGTTTGATCAGCAACAATTGCTTGCTTATTATAATTTTTTAACAGTATGTAAATGGTCTGT |
| | AGTTGTTAACGGTCCATTTTTTTCTTTTGAACAGTCTCATAATAATTGTTATGTGAATGTAGCTTGT |
| | CTTATGTTGCAGCATATTAATCTTAAATTTAATAAATGGCAGTGGCAGGAAGCATGGTATGAATTTC |
| | GTGCTGGCAGACCACATAGGTTAGTTGCTCTTGTTTTAGCTAAAGGTCATTTTAAATTTGATGAACC |
| | ATCAGATGCTACTGATTTTATTCGTGTTGTTTTGAAACAAGCTGATTTATCAGGTGCAATTTGTGAA |
| | TTAGAACTTATTTGTGATTGTGGTATTAAACAAGAAAGTCGTGTTGGTGTTGATGCTGTTATGCATT |
| | TTGGTACATTAGCAAAGACTGATCTTTTTAATGGTTATAAGATTGGCTGTAATTGTGCAGGTAGAAT |
| | TGTCCATTGTACTAAATTGAATGTACCATTTTTGATTTGTTCTAATACTCCTCTGAGTAAGGATTTA |
| | CCTGATGATGTTGTTGCAGCTAACATGTTTATGGGTGTAGGTGTAGGCCATTATACACATTTGAAAT |
| | GTGGTTCACCTTACCAACATTATGATGCTTGTAGTGTTAAAAAATATACAGGTGTTAGTGGTTGTTT |
| | AACTGACTGCTTGTATCTTAAAAATTTAACCCAGACTTTTACATCTATGTTGACTAATTATTTTTTG |
| | GATGATGTTGAAATGGTTGCTTATAACCCTGATCTTTCACAATATTATTGTGATAATGGTAAGTATT |
| | ATACAAAACCTATTATAAAGGCTCAGTTTAAACCATTTGCTAAAGTTGACGGTGTTTATACTAACTT |
| | TAAGTTAGTTGGACATGATATTTGTGCTCAATTGAATGATAAGTTAGGTTTTAATGTAGATTTGCCG |
| | TTTGTTGAGTACAAAGTAACAGTCTGGCCTGTAGCTACTGGTGATGTTGTTTTGGCATCTGATGATT |
| | TATATGTGAAACGTTATTTTAAAGGATGTGAAACTTTTGGTAAGCCTGTTATTTGGTTTTGTCATGA |
| | TGAAGCATCATTGAATTCTCTTACTTATTTTAATAAACCTAGTTTTAAATCTGAAAATAGATATAGT |
| | GTTTTGTCTGTTGATTCTGTATCTGAGGAGTCACAAGGTAATGTGGTTACTTCTGTTATGGAATCGC |
| | AGATTAGTACTAAAGAGGTTAAGTTAAAGGGTGTTAGAAAGACTGTTAAAATAGAAGATGCTATTAT |
| | TGTTAATGATGAAAATAGTTCTATTAAGGTTGTTAAAAGTTTATCTTTAGTTGATGTTTGGGATATG |
| | TATTTGACAGGTTGTGATTATGTTGTTTGGGTTGCTAATGAATTGTCACGCCTAGTTAAATCACCAA |
| | CAGTTAGGGAATATATACGATATGGTATTAAACCTATTACTATACCTATAGATTTGTTATGTTTAAG |
| | AGATGATAATCAAACTCTTTTAGTTCCTAAAATTTTTAAAGCAAGAGCTATAGAATTTTATGGTTTT |
| | TTGAAGTGGTTGTTTATTTATGTTTTTAGTTTATTACATTTTACAAATGATAAAACCATTTTTTATA |
| | CTACAGAAATAGCTTCTAAGTTTACTTTTAATTTGTTTTGTTTGGCTCTTAAAAATGCTTTTCAGAC |
| | ATTTAGATGGAGTATATTTATAAAAGGTTTTCTTGTTGTAGCCACTGTGTTTTTGTTTTGGTTTAAT |
| | TTTTTGTATATAAATGTTATTTTTAGTGACTTTTATCTTCCTAATATTAGTGTTTTTCCTATTTTTG |
| | TGGGAAGAATTGTTATGTGGATAAAGGCTACTTTTGGTTTGGTTACAATTTGTGATTTTTATTCTAA |
| | GTTAGGTGTAGGTTTTACAAGTCATTTTTGTAATGGTAGTTTTATATGTGAATTGTGTCATTCTGGT |
| | TTTGATATGTTGGATACATATGCAGCTATAGATTTTGTTCAGTATGAAGTAGATAGACGTGTTTTAT |
| | TTGATTATGTTAGTTTAGTCAAATTAATTGTTGAACTCGTTATTGGTTATTCATTATACACAGTATG |
| | GTTTTATCCATTATTTTGTCTTATTGGTTTACAATTATTTACTACATGGTTGCCTGATTTGTTTATG |
| | TTAGAAACTATGCATTGGTTGATTAGATTTATTGTATTTGTAGCTAATATGTTACCTGCTTTTGTCT |
| | TGTTGCGGTTTTATATAGTTGTTACTGCTATGTATAAAGTAGTTGGTTTTATTAGGCATATTGTCTA |
| | TGGTTGTAATAAAGCTGGTTGTTTATTTTGTTATAAACGAAATTGTAGTGTTCGTGTTAAGTGTAGT |
| | ACTATTGTTGGTGGTGTAATTCGTTATTATGATATTACTGCTAATGGTGGTACTGGTTTTTGTGTTA |
| | AACATCAATGGAATTGTTTTAATTGCCATTCTTTTAAACCAGGTAACACTTTTATAACTGTAGAAGC |
| | TGCTATAGAACTTTCTAAAGAGCTTAAACGACCTGTAAATCCAACTGATGCTTCACATTATGTAGTT |
| | ACTGATATTAAGCAAGTTGGTTGTATGATGCGTTTGTTCTATGATAGAGATGGACAGCGTGTTTACG |
| | ATGATGTTGATGCTAGTTTATTTGTAGATATTAATAATCTGTTACATTCTAAAGTTAAAGTTGTTCC |
| | TAATTTGTATGTAGTTGTAGTAGAGAGTGATGCTGATAGAGCTAATTTTCTGAATGCTGTTGTGTTT |
| | TATGCACAATCATTGTATAGGCCTATATTACTTGTAGACAAAAAGTTAATTACTACAGCTTGTAATG |
| | GTATCTCTGTAACCCAGACTATGTTTGATGTTTATGTTGATACTTTTATGTCTCATTTTGATGTTGA |
| | TAGAAAGAGTTTTAATAATTTTGTTAACATTGCTCATGCTTCTCTTAGAGAGGGTGTGCAATTAGAA |
| | AAGGTTTTAGATACTTTTGTGGGATGTGTACGTAAATGTTGTTCCATTGATTCAGATGTTGAAACAA |
| | GATTTATTACTAAATCTATGATATCTGCAGTAGCTGCTGGTTTGGAATTTACTGATGAAAATTATAA |
| | CAATTTGGTACCTACATATTTAAAGAGTGATAATATTGTAGCTGCTGATTTAGGTGTTCTTATACAG |
| | AATGGTGCTAAGCATGTACAGGGTAATGTTGCTAAGGCAGCTAATATTTCTTGTATATGGTTTATTG |
| | ATGCTTTTAATCAACTTACTGCTGATTTACAGCATAAATTAAAAAAAGCATGTGTTAAAACTGGCTT |
| | GAAGTTAAAATTGACTTTTAATAAGCAAGAGGCAAGTGTCCCTATTCTTACAACACCCTTTTCACTT |
| | AAAGGAGGTGTTGTATTGAGTAATTTGTTATATATATTATTTTTTGTTAGTTTAATCTGTTTTATAT |
| | TATTGTGGGCTTTATTGCCTACATATAGTGTTTATAAGTCTGATATTCATTTGCCTGCTTATGCTAG |
| | TTTTAAAGTTATTGATAATGGTGTTGTTAGAGATATTTCAGTTAATGATTTATGTTTTGCTAATAAA |
| | TTTTTCCAATTTGATCAATGGTATGAGTCCACTTTTGGGTCTGTTTACTATCATAATTCTATGGATT |
| | GCCCTATTGTAGTGGCAGTTATGGATGAAGATATCGGTTCTACTATGTTTAATGTTCCTACTAAAGT |
| | TTTGAGACATGGCTTTCATGTTTTACATTTTTTAACTTATGCATTTGCTAGTGATAGTGTTCAGTGC |
| | TATACACCACATATTCAGATTTCTTATAATGATTTTTATGCTAGTGGTTGTGTTTTATCATCTTTGT |
| | GTACTATGTTTAAAAGAGGTGATGGTACACCACATCCTTATTGTTATTCAGATGGTGTTATGAAGAA |
| | TGCTTCTTTGTATACATCTTTGGTTCCACATACACGTTATAGCCTTGCTAATTCTAATGGTTTTATA |
| | AGATTTCCTGATGTTATTAGTGAAGGTATTGTACGTATTGTAAGAACGCGCTCTATGACTTATTGTA |
| | GAGTGGGTGCATGTGAATACGCCGAAGAGGGTATATGTTTTAATTTTAATAGTTCCTGGGTTTTGAA |
| | TAATGATTATTATAGAAGTATGCCTGGAACTTTTTGTGGTAGAGATCTTTTTGATTTGTTTTATCAA |
| | TTTTTTAGTAGTTTAATTCGTCCTATAGATTTCTTTTCTCTTACTGCTAGTTCTATTTTTGGAGCTA |
| | TATTGGCTATAGTTGTTGTCTTGGTTTTTTATTATTTAATAAAACTTAAGCGTGCTTTTGGAGATTA |
| | TACTAGTGTTGTAGTTATAAATGTTGTTGTTTGGTGTATTAATTTTCTTATGCTTTTTGTTTTTCAA |
| | GTTTATCCTATTTGTGCATGTGTTTATGCTTGTTTTTATTTTTATGTAACATTGTATTTTCCTTCTG |
| | AAATTAGTGTAATTATGCATTTGCAATGGATTGTTATGTATGGTGCTATAATGCCTTTTTGGTTTTG |
| | TGTCACATATGTAGCTATGGTTATTGCAAACCATGTTTTATGGTTATTTTCATATTGTAGGAAAATT |
| | GGTGTTAATGTATGTAGTGATAGTACATTTGAAGAAACATCTCTTACTACTTTTATGATTACTAAAG |
| | ATTCTTATTGTAGATTAAAGAATTCTGTTTCTGATGTTGCCTACAATAGATATTTGAGTTTGTATAA |
| | TAAGTATCGTTACTATAGTGGTAAAATGGATACTGCTGCCTATAGAGAAGCGGCGTGTTCTCAGTTA |
| | GCTAAAGCTATGGAAACATTTAATCACAATAATGGTAATGATGTCTTATACCAACCTCCTACAGCAT |
| | CTGTTTCTACATCTTTTTTGCAATCAGGTATTGTAAAGATGGTATCTCCTACGTCAAAAATTGAACC |
| | TTGTATTGTTAGTGTTACTTATGGTAGTATGACTTTGAATGGTTTATGGTTAGATGACAAAGTTTAT |
| | TGTCCTCGTCATGTTATATGTTCATCCTCTAATATGAACGAACCTGATTATTCTGCCTTATTGTGTA |
| | GAGTTACTCTAGGTGATTTTACTATAATGTCTGGTCGGATGAGTTTAACAGTTGTGTCTTACCAGAT |
| | GCAGGGCTGTCAACTTGTTTTGACAGTCTCTTTACAAAATCCTTACACTCCAAAATATACTTTTGGT |
| | AATGTTAAACCTGGTGAAACTTTTACTGTTTTAGCTGCGTATAATGGCCGACCACAAGGGGCATTTC |
| | ATGTTACTATGCGTAGTAGTTATACTATTAAAGGTTCTTTTTTGTGTGGGTCATGTGGATCTGTTGG |
| | TTATGTATTAACAGGTGATAGTGTTAAGTTTGTATATATGCATCAATTAGAGCTCAGTACTGGTTGT |
| | CACACTGGCACTGATTTTACTGGTAATTTTTATGGTCCATATAGAGATGCTCAAGTTGTACAGTTGC |
| | CAGTTAAGGACTACGTCCAGACTGTTAATGTTATTGCTTGGCTCTATGCAGCTATACTTAATAATTG |
| | TGCTTGGTTTGTACAAAATGATGTTTGTTCTACTGAAGATTTTAATGTTTGGGCTATGGCAAATGGT |
| | TTTAGCCAAGTAAAAGCAGATCTTGTCTTAGATGCTTTGGCTTCAATGACAGGTGTTTCTATTGAAA |
| | CTTTATTGGCTGCTATTAAGCGTCTATATATGGGATTTCAAGGTCGTCAAATACTAGGAAGTTGTAC |
| | TTTTGAAGATGAATTGGCACCTTCTGACGTTTATCAACAATTGGCTGGTGTTAAATTGCAATCTAAA |
| | ACAAAAAGATTTATTAAAGAAACAATTTATTGGATTTTGATATCTAGATTTTTGTTTAGTTGTATAA |
| | TTTCTGCATTTGTTAAATGGACTATATTTATGTATATTAATACACATATGATTGGTGTTACATTATG |
| | TGTACTTTGTTTTGTTAGTTTTATGATGTTACTAGTTAAACATAAGCATTTTTATTTGACTATGTAT |
| | ATAATTCCTGTACTCTGTACCTTGTTTTATGTAAATTATTTAGTTGTTTATAAGGAAGGTTTTAGAG |
| | GTTTTACTTATGTCTGGCTCTCATATTTTGTTCCTGCTGTGAATTTTACTTATGTTTATGAAGTATT |
| | TTATGGTTGTATTTTATGTGTTTTTGCTATTTTTATAACTATGCATAGTATTAATCATGACATTTTT |
| | TCTTTGATGTTTTTGGTTGGTAGAATAGTTACTTTAATTTCTATGTGGTATTTTGGGTCGAATTTAG |
| | AAGAGGATGTTTTGTTATTTATTACAGCCTTTTTAGGTACTTATACATGGACCACTATTTTGTCATT |
| | AGCTATAGCAAAAATTGTTGCTAATTGGTTGTCTGTTAATATATTTTATTTTACAGATGTACCTTAT |
| | ATTAAATTGATTCTCTTGAGTTACTTATTTATAGGGTATATTTTATCTTGTTATTGGGGATTTTTCT |
| | CTCTTTTAAACAGTGTTTTTAGAATGCCTATGGGTGTTTATAATTATAAAATTTCTGTTCAAGAATT |
| | GCGTTATATGAATGCTAATGGCTTACGTCCACCTCGTAATAGTTTTGAGGCTATTTTGTTAAATTTA |
| | AAACTGCTTGGAATAGGTGGCGTGCCAGTTATTGAAGTCTCCCAAATTCAATCAAAATTGACTGATG |
| | TGAAATGTGCTAATGTTGTTTTGTTAAATTGTTTACAGCATTTGCATGTTGCTTCTAATTCTAAGTT |
| | GTGGCAGTATTGTAGTGTTTTACATAATGAAATACTATCTACTTCAGATTTGAGTGTAGCTTTTGAT |
| | AAGCTTGCTCAATTATTGATTGTTTTATTCGCCAATCCTGCTGCAGTTGATACTAAGTGTCTTGCAA |
| | GTATAGATGAAGTTAGCGATGATTATGTTCAAGATAGTACCGTTTTGCAGGCTTTGCAAAGTGAGTT |
| | TGTAAATATGGCTAGTTTTGTTGAATATGAAGTCGCAAAGAAAAATTTGGCTGATGCTAAAAATAGT |
| | GGTTCTGTTAATCAACAACAGATAAAACAGTTAGAAAAAGCATGTAATATAGCTAAGTCTGTGTATG |
| | AACGTGATAAAGCTGTAGCTCGCAAACTTGAACGTATGGCAGACCTAGCACTTACTAACATGTATAA |
| | AGAGGCTCGGATTAATGATAAGAAGAGTAAAGTTGTTTCCGCTTTGCAGACAATGCTTTTTAGCATG |
| | GTTCGTAAATTGGATAATCAGGCTTTAAATTCTATTCTGGATAATGCTGTTAAAGGTTGTGTACCTT |
| | TGAGTGCTATTCCAGCATTGGCTGCTAATACTTTAACTATAGTAATACCAGATAAACAAGTTTTTGA |
| | TAAAGTTGTTGATAATGTTTATGTTACATATGCTGGTAGTGTATGGCATATACAGACTGTTCAAGAT |
| | GCTGATGGTATTAATAAACAGTTAACTGATATTAGTGTTGATTCTAATTGGCCTCTTGTTATCATTG |
| | CGAACAGGTATAATGAAGTTGCTAATGCTGTTATGCAGAATAATGAGTTGATGCCTCATAAATTAAA |
| | AATACAAGTTGTTAATAGTGGTTCTGATATGAATTGTAATATTCCTACTCAATGTTATTATAATAAT |
| | GGTAGTAGTGGTAGAATAGTTTATGCTGTTCTTAGTGATGTTGATGGTCTTAAGTATACTAAGATAA |
| | TGAAAGATGATGGAAATTGTGTTGTTTTAGAGCTTGATCCTCCTTGTAAATTTTCTATACAAGATGT |
| | TAAGGGACTTAAAATTAAGTATCTTTATTTTATTAAAGGATGTAACACTTTAGCTAGAGGGTGGGTT |
| | GTTGGTACTTTATCTTCAACAATTAGATTGCAGGCTGGTGTTGCTACTGAGTATGCAGCTAATTCTT |
| | CTATACTTTCATTATGTGCATTTTCTGTAGATCCTAAGAAAACTTATTTAGATTATATACAACAAGG |
| | TGGTGTACCTATAATTAATTGTGTTAAAATGCTCTGTGATCATGCTGGTACTGGTATGGCCATTACT |
| | ATTAAACCTGAGGCTACTATTAACCAAGATTCTTATGGTGGTGCCTCAGTTTGTATTTATTGCCGTG |
| | CACGTGTAGAGCATCCAGATGTAGATGGTATATGTAAATTACGTGGTAAATTTGTACAAGTCCCTTT |
| | GGGTATAAAAGATCCTATTCTTTATGTGTTAACACATGATGTTTGTCAAGTCTGTGGTTTTTGGAGA |
| | GATGGCAGTTGTTCCTGTGTAGGTTCAAGTGTCGCTGTTCAATCTAAAGATTTAAATTTTTTAAACG |
| | GGTTCGGGGTACTAGTGTGAATGCCCGGCTAGTACCCTGTGCTAGTGGTTTATCTACTGATGTTCAA |
| | TTAAGGGCATTTGACATTTGTAATACCAATAGAGCTGGTATAGGTTTATATTATAAAGTGAATTGTT |
| | GCCGTTTTCAGCGTATAGATGACGACGGTAATAAATTGGATAAGTTCTTTGTTGTCAAAAGAACTAA |
| | TTTAGAAGTTTATAATAAAGAGAAAACTTATTATGAGTTGACTAAAAGTTGTGGTGTTGTGGCTGAA |
| | CATGATTTCTTTACATTTGATATTGATGGTAGTCGCGTGCCACATATAGTTCGTAGGAATCTTTCAA |
| | AGTATACTATGTTAGATCTTTGCTATGCATTGCGTCATTTTGATCGTAATGATTGTTCAATATTGTG |
| | TGAAATTCTTTGTGAGTATGCTGATTGTAAAGAATCCTACTTTTCTAAGAAAGATTGGTATGATTTT |
| | GTTGAAAATCCTGATATTATTAATATATATAAAAAATTAGGCCCTATTTTTAATAGAGCTTTACTTA |
| | ATACTGTCATTTTTGCAGACACCTTAGTTGAAGTAGGTTTAGTTGGTGTTTTAACTTTAGATAACCA |
| | AGATTTGTATGGTCAATGGTATGATTTTGGTGATTTTATACAAACAGCCCCAGGGTTTGGTGTGGCA |
| | GTTGCAGATTCTTACTATTCTTATATGATGCCTATGTTGACTATGTGTCATGTATTAGATTGTGAAT |
| | TATTTGTTAATGATAGTTATAGACAATTCGATCTTGTACAGTATGATTTTACTGATTACAAGTTAGA |
| | GTTGTTTAATAAGTATTTTAAGTATTGGGGTATGAAGTATCATCCTAATACTGTGGATTGTGATAAT |
| | GATAGGTGTATTATTCATTGTGCTAATTTTAATATACTATTTAGTATGGTTTTACCTAATACTTGTT |
| | TTGGTCCCCTTGTTAGACAAATTTTTGTAGATGGTGTACCGTTTGTTGTTTCTATTGGTTACCATTA |
| | CAAAGAGTTAGGTGTAGTTATGAACTTAGATGTTGACACACACCGTTATCGTTTGTCTCTTAAAGAT |
| | TTACTTCTTTATGCAGCAGATCCTGCTATGCACGTTGCATCTGCTAGTGCTCTGCTTGATTTACGAA |
| | CTTGTTGTTTTAGTGTAGCTGCCATTACAAGTGGTATAAAATTTCAAACTGTAAAACCAGGTAACTT |
| | TAACCAAGACTTTTACGAGTTTGTTAAAAGTAAAGGCTTGTTTAAAGAGGGTAGTACAGTTGATTTG |
| | AAACATTTTTTCTTTACTCAAGATGGTAATGCTGCAATTACTGATTATAATTATTATAAGTATAATT |
| | TACCTACTATGGTTGATATTAAGCAGTTATTGTTTGTATTAGAAGTTGTTTATAAATATTTTGAAAT |
| | TTATGATGGTGGTTGTATACCAGCATCACAAGTTATTGTTAATAATTATGATAAAAGTGCTGGTTAT |
| | CCATTTAATAAATTTGGTAAAGCCAGACTTTATTATGAGGCATTATCATTTGAGGAACAGAATGAAA |
| | TTTATGCATATACTAAACGTAATGTTCTGCCCACCTTAACTCAAATGAATTTAAAATATGCTATCAG |
| | TGCTAAGAATAGAGCTCGCACTGTAGCAGGTGTTTCTATTCTTAGTACTATGACAGGCCGAATGTTC |
| | CATCAAAAATGTTTGAAGAGTATAGCAGCTACCCGAGGTGTTCCTGTTGTTATAGGAACCACTAAAT |
| | TTTATGGTGGTTGGGACGATATGTTACGTCATCTTATAAAGGATGTTGACAACCCTGTTCTTATGGG |
| | TTGGGATTATCCTAAATGTGATCGTGCTATGCCAAATATTTTGCGTATTGTTAGTAGTTTAGTTTTG |
| | GCCCGCAAACATGAATTTTGTTGTTCACATGGTGATAGATTTTATCGCCTTGCGAATGAATGTGCTC |
| | AAGTTTTGAGTGAAATAGTTATGTGTGGCGGTTGCTATTATGTTAAGCCTGGTGGTACTAGCAGTGG |
| | TGATGCAACTACTGCTTTTGCTAATTCTGTTTTTAATATATGTCAGGCTGTTACTGCTAATGTTTGT |
| | TCTCTTATGGCCTGTAATGGCCATAAGATTGAAGATTTAAGTATACGCAATTTACAAAAACGCTTAT |
| | ACTCTAATGTTTATCGTACAGATTATGTTGATTATACATTTGTTAATGAGTATTATGAATTTTTATG |
| | TAAGCATTTTAGTATGATGATTTTGAGTGATGATGGTGTTGTCTGTTATAACTCTGATTATGCTAGT |
| | AAGGGTTATATAGCTAATATAAGTGTTTTTCAACAAGTTTTGTACTATCAGAATAATGTCTTTATGT |
| | CTGAATCTAAATGTTGGGTTGAAAATGATATTACTAATGGTCCTCATGAATTTTGTTCCCAACATAC |
| | TATGTTAGTTAAGATAGATGGTGATTATGTTTATTTACCATATCCAGATCCTTCTAGAATTTTAGGA |
| | GCTGGTTGTTTTGTTGATGATTTATTGAAGACTGACAGTGTTCTTTTGATAGAGCGCTTTGTAAGTC |
| | TAGCTATAGATGCTTACCCTTTAGTACATCATGAAAATGAAGAATACCAAAAAGTCTTTCGTGTATA |
| | TTTAGAATATATAAAAAAACTGTATAATGATCTTGGTACTCAGATCTTAGATAGTTATAGTGTTATT |
| | TTAAGTACTTGTGATGGTTTAAAGTTTACTGAAGAATCATTTTACAAGAATATGTATTTAAAAAGTG |
| | CCGTGATGCAGAGTGTAGGTGCATGCGTTGTTTGTTCATCACAAACTTCTTTGCGTTGTGGCAGTTG |
| | TATACGTAAGCCTTTGTTATGTTGTAAATGTTGTTATGACCATGTTATGGCAACTAATCATAAATAT |
| | GTTTTGAGTGTCTCACCTTACGTTTGTAATGCACCTAACTGTGATGTGAGTGATGTCACCAAATTAT |
| | ATTTGGGCGGTATGTCTTACTATTGTGAAAACCATAAACCCCATTATTCATTTAAGTTAGTTATGAA |
| | TGGTATGGTCTTTGGTTTGTATAAACAATCTTGCACGGGTTCACCTTATATAGATGATTTTAATAAG |
| | ATAGCTAGTTGTAAATGGACAGAAGTTGATGATTATGTTCTGGCAAATGAGTGTATTGAACGTTTAA |
| | AGTTATTTGCTGCAGAAACTCAAAAGGCAACTGAAGAGGCTTTTAAACAAAGCTATGCTTCTGCTAC |
| | CATTCAAGAGATTGTTAGTGATAGAGAAGTTATTTTGTGTTGGGAGACAGGTAAAGTTAAACCACCA |
| | CTTAATAAAAATTATGTTTTCACAGGCTACCATTTTACTAGTACTGGTAAGACAGTTTTAGGTGAGT |
| | ATGTTTTTGATAAAAGTGAATTAACTAACGGTGTGTATTACCGCGCTACAACTACTTATAAACTTTC |
| | TATAGGTGATGTTTTTGTTTTAACATCACATTCTGTAGCTAGTTTAAGTGCACCTACACTTGTCCCA |
| | CAAGAGAACTATGCTAGTATAAGATTTTCTAGTGTTTATAGTGTTCCATTGGTGTTTCAAAATAATG |
| | TTGCTAATTATCAGCACATTGGAATGAAACGTTATTGCACTGTTCAAGGTCCCCCTGGTACGGGAAA |
| | GTCTCATCTTGCTATAGGTCTAGCTGTTTATTACTACACAGCACGTGTAGTTTATACTGCTGCTAGT |
| | CATGCTGCTGTAGATGCATTGTGTGAAAAAGCTTATAAGTTTTTAAATATTAACGATTGTACACGTA |
| | TTATTCCTGCTAAAGTTCGTGTAGATTGTTATGATAAGTTTAAAATTAATGATACCACTTGTAAGTA |
| | TGTTTTTACCACAATAAATGCATTACCAGAGTTGGTTACAGATATTGTTGTTGTTGATGAAGTTAGT |
| | ATGCTTACTAATTATGAATTGTCTGTTATAAATGCTCGTATTAAAGCTAAACATTATGTATATATTG |
| | GAGATCCTGCTCAATTACCTGCACCACGTGTGCTGTTGAGCAAGGGTTCTTTAGAACCTAGGCACTT |
| | CAATTCTATTACTAAAATAATGTGTTGTTTAGGTCCTGATATCTTTTTGGGAAATTGTTATAGGTGT |
| | CCTAAAGAAATTGTAGAAACTGTTTCAGCATTGGTTTATGATAATAAACTCAAGGCTAAAAATGATA |
| | ATAGTTCATTATGTTTTAAAGTATATTTTAAGGGACAGACAACACATGAGAGTTCAAGTGCTGTAAA |
| | TATTCAACAGATATATCTAATTAGTAAATTTTTAAAAGCTAATCCAGTTTGGAATAGTGCTGTTTTT |
| | ATTAGTCCTTATAATAGTCAGAATTATGTTGCTAAGCGTGTTTTAGGTGTTCAAACACAAACTGTAG |
| | ATTCTGCTCAAGGTTCGGAATATGATTATGTTATATATTCACAAACAGCAGAAACAGCCCATTCTGT |
| | TAATGTTAATCGATTTAATGTTGCCATAACTAGAGCCAAGAAGGGCATTTTTTGTGTTATGAGTAAT |
| | ATGCAATTATTTGAATCTCTTAATTTTATTACTCTACCTTTAGATAAAATTCAAAATCAAACTTTAC |
| | CTCGTTTGCATTGCACAACTAATCTTTTTAAAGATTGTAGTAAAAGTTGCTTAGGTTATCATCCAGC |
| | GCATGCCCCCTCATTTTTAGCAGTTGATGATAAATATAAGGTTAATGAAAATTTGGCTGTAAATTTA |
| | AATATTTGTGAACCTGTTTTAACATATTCTCGTTTAATATCTCTTATGGGTTTTAAATTAGATTTGA |
| | CTCTTGATGGTTATTCTAAATTGTTTATTACTAAAGATGAAGCCATTAAACGTGTTAGAGGTTGGGT |
| | TGGTTTTGATGTTGAGGGCGCTCATGCTACTCGCGAAAACATTGGAACAAACTTTCCACTGCAAATA |
| | GGTTTTTCAACTGGTGTGGATTTTGTAGTTGAAGCTACTGGCTTATTTGCTGAGAGAGATTGTTATA |
| | CTTTTAAAAAAACTGTAGCTAAAGCTCCTCCTGGTGAAAAATTTAAACATTTAATACCCCTTATGTC |
| | AAAAGGTCAAAAGTGGGATATTGTTAGAATTAGAATTGTTCAAATGTTATCTGATTATCTTTTAGAC |
| | CTTTCTGATAGTGTAGTATTTATTACTTGGTCTGCCAGTTTTGAACTTACTTGTTTAAGGTATTTTG |
| | CTAAATTAGGCAGAGAGCTTAATTGTAATGTGTGTTCTAATCGTGCTACATGCTACAATTCTAGAAC |
| | TGGTTATTATGGTTGTTGGCGCCATAGTTATACTTGTGATTATGTGTATAATCCACTTATTGTAGAT |
| | ATACAACAGTGGGGTTATACAGGTTCTTTAACTAGTAATCACGATATAATTTGTAATGTACATAAAG |
| | GTGCACATGTTGCGTCAGCTGATGCAATTATGACTCGTTGTTTAGCAATCTATGATTGTTTTTGTAA |
| | ATCTGTTAATTGGAATTTAGAGTATCCAATAATTTCTAATGAGGTCAGTATAAATACATCTTGTAGG |
| | TTATTGCAGCGTGTCATGCTTAAAGCTGCCATGCTATGTAATAGATACAACTTATGTTATGACATAG |
| | GCAATCCTAAAGGTTTAGCTTGTGTCAAAGATTATGAATTTAAATTTTATGATGCTTTTCCTGTAGC |
| | CAAGTCTGTTAAACAGTTATTTTATGTCTATGATGTGCATAAAGATAATTTTAAAGATGGTTTATGT |
| | ATGTTTTGGAATTGTAATGTTGATAAATATCCATCTAATTCAATTGTTTGTAGATTTGACACTCGAG |
| | TGTTAAATAAATTAAACCTTCCTGGATGTAATGGTGGTAGTTTGTATGTTAATAAACATGCATTCCA |
| | TACTAATCCTTTTACTAGAACTGTTTTTGAAAATCTTAAGCCTATGCCTTTTTTCTATTATTCAGAT |
| | ACGCCTTGTGTGTACGTAGATGGTTTAGAATCTAAACAAGTTGATTACGTTCCTTTAAGAAGCGCCA |
| | CTTGTATCACACGGTGTAATCTAGGTGGAGCTGTTTGTTCAAAGCATGCTGAAGAATATTGTAACTA |
| | CCTTGAGTCTTATAATATAGTTACTACAGCAGGCTTTACTTTTTGGGTTTATAAGAATTTTGATTTT |
| | TATAATTTATGGAACACTTTTACTACGTTACAGAGTTTAGAAAACGTAATATATAACTTGGTTAATG |
| | TTGGTCATTATGATGGACGTACAGGTGAATTACCTTGTGCTATTATGAATGACAAAGTTGTTGTTAA |
| | GATTAATAATGTAGATACTGTTATTTTTAAAAATAATACATCATTTCCTACTAATATAGCTGTTGAA |
| | TTGTTTACAAAACGTAGTATCCGGCACCACCCTGAACTTAAGATTCTTAGAAATTTGAACATTGATA |
| | TTTGTTGGAAGCATGTCCTGTGGGATTATGTTAAAGATAGTTTGTTTTGTAGTTCCACTTATGGTGT |
| | TTGTAAATACACAGATTTGAAGTTCATCGAAAATTTGAATATACTTTTTGATGGTCGTGACACTGGC |
| | GCTTTAGAAGCTTTTAGAAAAGCAAGAAATGGTGTTTTTATTAGTACTGAAAAATTAAGTAGGTTAT |
| | CAATGATTAAAGGTCCGCAACGAGCTGATTTAAATGGTGTGATTGTGGATAAAGTTGGAGAACTCAA |
| | AGTTGAGTTTTGGTTCGCTATGAGAAAAGATGGTGACGATGTTATCTTCAGCCGAACAGACAGCCTA |
| | TGCTCAAGCCATTACTGGAGCCCACAAGGTAATCTAGGTGGTAATTGCGCGGGTAATGTCATTGGTA |
| | ATGATGCTCTAACACGTTTTACTATCTTTACTCAGAGTCGTGTATTGTCAAGTTTTGAACCTCGCTC |
| | AGATTTAGAACGGGATTTTATTGATATGGATGATAATCTGTTTATTGCTAAATATGGTTTAGAAGAC |
| | TATGCATTTGATCATATAGTTTATGGTAGTTTTAACCATAAAGTTATAGGAGGTTTGCATTTGCTTA |
| | TAGGCTTATTTCGTAGGAAAAAAAAATCTAATTTGTTAATTCAAGAGTTTTTACAGTATGATTCTAG |
| | TATTCATTCATATTTTATTACTGATCAGGAGTGTGGTAGTAGTAAGAGTGTTTGTACAGTTATTGAT |
| | TTATTATTAGATGATTTTGTTTCTATTGTTAAGTCATTAAATTTGAGTTGTGTTAGTAAAGTTGTTA |
| | ATATTAATGTTGATTTTAAGGATTTTCAATTTATGTTGTGGTGTAATGATAATAAAATTATGACTTT |
| | TTATCCTAAAATGCAAGCCACTAATGATTGGAAACCTGGCTATTCTATGCCTGTTTTGTATAAGTAT |
| | TTGAATGTTCCATTAGAGAGAGTCTCTTTATGGAATTATGGTAAACCTATTAATTTGCCTACAGGCT |
| | GTATGATGAATGTTGCTAAGTACACTCAATTATGTCAGTATTTGAATACTACAACATTAGCTGTTCC |
| | TGTTAATATGCGTGTTTTACATTTAGGTGCAGGGTCTGATAAAGAAGTAGCTCCAGGTTCTGCTGTT |
| | TTAAGACAGTGGTTACCATCTGGTAGTATTCTTGTAGATAATGATTTAAACCCATTTGTTAGCGATA |
| | GTTTAGTTACTTATTTTGGAGATTGTATGACTTTACCATTTGATTGTCATTGGGATTTGATAATATC |
| | TGATATGTATGATCCTCTTACTAAAAATATTGGTGATTATAATGTGAGTAAGGATGGGTTTTTTACT |
| | TACATTTGTCATTTAATTCGTGATAAATTATCTTTGGGTGGTAGTGTAGCTATAAAAATTACAGAGT |
| | TTTCTTGGAATGCTGATTTATATAAATTAATGAGTTGTTTTGCATTTTGGACAGTTTTTTGTACTAA |
| | TGTAAATGCTTCTTCTAGTGAAGGGTTTTTAATAGGTATAAATTACCTGGGTAAATCTTCTTTTGAA |
| | ATAGATGGCAATGTTATGCATGCTAACTATTTGTTTTGGAGAAATAGTACAACATGGAATGGCGGTG |
| | CTTATAGTTTATTTGATATGACTAAATTTTCTTTGAAATTGGCTGGCACTGCTGTTGTTAATTTAAG |
| | ACCAGATCAATTAAATGATTTAGTTTATTCTCTTATTGAAAGAGGTAAATTATTAGTTCGCGATACG |
| | CGTAAAGAGATTTTTGTTGGTGATAGTCTTGTAAATACTTGTTAGATCTCATTAAATCTAAACTATG |
| | TTAATTATTTTTTTATTTTTTTATTTCTGTTATGGTTTTAATGAACCTCTTAATGTTGTGTCTCATT |
| | TAAACCATGACTGGTTTTTATTTGGTGATAGTCGTTCTGATTGTAACCATATTAATAATTTAAAAAT |
| | TAAAAATTTTGATTATTTGGATATTCACCCTAGTTTGTGCAACAATGGTAAGATTTCATCTAGTGCC |
| | GGTGATTCTATTTTTAAGAGTTTTCATTTCACTCGATTTTATAATTACACTGGCGAAGGTGATCAAA |
| | TTATTTTTTATGAGGGTGTTAATTTTAATCCTTATCATAGATTTAAGTGTTTTCCTAATGGTAGTAA |
| | TGATGTATGGCTTCTTAACAAGGTAAGATTTTATCGTGCCTTATATTCTAATATGGCCTTTTTTCGT |
| | TATCTTACTTTTGTTGATATTCCTTATAATGTTTCTCTTTCTAAGTTTAATTCTTGTAAAAGTGATA |
| | TTTTATCACTTAACAATCCTATTTTTATTAATTATTCTAAGGAAGTTTATTTTACTTTATTAGGTTG |
| | TTCTCTTTATTTAGTACCGCTTTGCCTTTTTAAATCTAACTTTAGTCAGTACTATTATAACATAGAT |
| | ACTGGCTCTGTTTATGGTTTTTCTAATGTTGTTTATCCTGATTTAGACTGTATTTATATTTCTCTTA |
| | AACCAGGTTCTTATAAAGTTTCCACCACTGCACCTTTTTTATCCTTACCTACTAAAGCTCTCTGTTT |
| | TGATAAATCTAAACAATTTGTACCTGTACAGGTTGTTGATTCTAGATGGAACAACGAGCGTGCCTCA |
| | GATATTTCTTTATCTGTTGCATGTCAATTGCCATATTGTTATTTTCGCAATTCTTCTGCTAATTATG |
| | TTGGCAAGTATGATATTAACCACGGTGATAGTGGTTTTATTTCTATTTTATCTGGTCTTTTATATAA |
| | TGTTTCTTGTATTTCATATTATGGTGTATTTTTATATGATAATTTTACATCCATTTGGCCCTATTAT |
| | TCTTTTGGTAGGTGTCCTACATCTTCTATTATTAAACATCCAATTTGTGTTTATGATTTTTTGCCTA |
| | TTATTTTACAAGGTATTTTATTATGTTTAGCTTTACTTTTTGTTGTTTTTCTATTATTTTTGTTATA |
| | TAACGATAAATCTCATTAAATCTAAACATGTTATTAATTATTTTTATTTTGCCTACAACATTAGCTG |
| | TTATAGGTGATTTTAATTGTACTAATTTTGCTATTAATGATTTAAACACCACAGTTCCTCGCATAAG |
| | TGAGTATGTTGTGGATGTTTCTTATGGTTTGGGTACATATTATATACTTGATCGTGTTTATTTAAAT |
| | ACTACTATATTATTTACTGGTTATTTCCCTAAATCTGGTGCCAATTTTAGGGATCTATCTTTAAAAG |
| | GTACTACATATTTGAGTACTCTTTGGTATCAGAAACCCTTTTTATCTGATTTTAATAATGGTATTTT |
| | TTCTAGAGTTAAGAATACTAAGTTGTATGTTAATAAAACTTTGTATAGTGAGTTTAGTACTATAGTT |
| | ATAGGTAGTGTTTTTATTAACAACTCTTATACTATTGTTGTTCAACCTCATAATGGTGTTTTGGAGA |
| | TTACAGCTTGTCAATACACTATGTGTGAGTATCCTCATACTATTTGTAAATCTAAAGGTAGTTCTCG |
| | TAATGAATCTTGGCATTTTGATAAATCTGAACCTTTGTGTCTGTTCAAGAAAAATTTTACTTATAAT |
| | GTTTCTACAGATTGGTTGTATTTTCATTTTTATCAAGAACGTGGCACTTTTTATGCTTATTATGCTG |
| | ATTCTGGCATGCCTACTACTTTTTTATTTAGTTTGTATCTTGGTACTCTTTTATCTCATTATTATGT |
| | TTTGCCTTTGACTTGTAATGCTATATCTTCTAATACTGATAATGAGACTTTACAATATTGGGTCACA |
| | CCTTTGTCTAAACGCCAATATCTTCTTAAATTTGACAACCGTGGTGTTATTACTAATGCTGTTGATT |
| | GTTCTAGTAGTTTCTTTAGCGAGATTCAATGTAAAACTAAATCTTTATTACCTAATACTGGTGTTTA |
| | TGACTTATCTGGTTTTACTGTTAAGCCTGTTGCAACTGTACATCGTCGTATTCCTGATTTACCTGAT |
| | TGTGACATTGATAAATGGCTTAACAATTTTAATGTACCCTCACCTCTTAATTGGGAACGTAAAATTT |
| | TTTCTAATTGCAACTTTAATTTGAGTACTTTGCTTCGTTTAGTTCATACTGATTCTTTTTCTTGTAA |
| | TAATTTTGATGAATCTAAGATATATGGTAGTTGTTTTAAGAGTATTGTTTTAGATAAATTTGCCATA |
| | CCCAACTCCAGACGATCTGATTTGCAGTTGGGCAGTTCTGGTTTTCTGCAATCTTCTAATTATAAAA |
| | TTGACACTACTTCTAGTTCTTGTCAATTGTATTATAGTTTGCCTGCAATTAATGTTACTATTAATAA |
| | TTATAATCCTTCTTCTTGGAATAGAAGGTATGGTTTTAATAATTTTAATTTGAGCTCTCATAGTGTT |
| | GTTTACTCACGTTATTGTTTTTCTGTTAATAATACTTTTTGTCCTTGTGCTAAACCTTCTTTTGCTT |
| | CAAGTTGCAAGAGTCATAAACCACCTTCTGCTTCCTGTCCTATTGGTACTAATTATCGTTCTTGTGA |
| | GAGTACTACTGTACTCGACCACACTGACTGGTGTAGGTGTTCTTGTTTACCTGATCCTATAACTGCT |
| | TATGACCCTAGGTCTTGTTCTCAAAAAAAGTCTCTGGTTGGTGTTGGTGAACATTGTGCAGGGTTCG |
| | GTGTTGATGAAGAAAAGTGTGGTGTATTGGATGGATCATATAATGTTTCTTGTCTTTGTAGTACTGA |
| | TGCCTTTCTAGGTTGGTCTTATGACACTTGCGTCAGTAACAACCGTTGTAATATTTTTTCTAATTTT |
| | ATTTTAAATGGTATCAATAGTGGTACCACTTGTTCTAATGATTTATTGCAGCCTAATACTGAAGTTT |
| | TTACTGATGTTTGTGTTGATTACGACCTTTATGGTATTACAGGACAAGGTATTTTTAAAGAAGTTTC |
| | TGCTGTTTATTATAATAGTTGGCAAAATCTTTTGTATGATTCTAATGGCAACATTATTGGTTTTAAA |
| | GATTTTGTTACTAATAAAACATATAATATTTTCCCTTGTTATGCAGGAAGAGTTTCTGCTGCTTTTC |
| | ATCAAAATGCTTCCTCTTTGGCTTTACTTTATCGTAATTTAAAATGTAGCTATGTTTTGAATAATAT |
| | TTCTTTAACTACTCAGCCATATTTTGATAGTTATCTTGGTTGCGTTTTTAATGCTGATAATTTAACT |
| | GATTATTCTGTTTCTTCTTGTGCTCTTCGCATGGGTAGTGGTTTTTGTGTTGATTATAACTCACCTT |
| | CTTCTTCCTCTTCGCGTCGTAAACGTAGAAGTATTTCTGCTTCTTATCGTTTTGTTACTTTTGAACC |
| | CTTTAATGTCAGTTTTGTTAATGACAGTATTGAGTCTGTGGGTGGTCTTTATGAGATCAAAATTCCC |
| | ACTAACTTTACTATAGTTGGTCAAGAGGAATTTATTCAAACTAATTCTCCTAAAGTTACTATTGATT |
| | GTTCTTTATTTGTCTGTTCTAATTATGCAGCTTGCCATGACTTATTGTCAGAGTATGGCACTTTTTG |
| | TGATAATATTAATAGTATTTTAGATGAAGTTAATGGTTTACTTGATACTACTCAATTGCATGTAGCT |
| | GATACTCTTATGCAAGGTGTCACACTTAGCTCCAATCTTAATACTAATTTGCATTTTGATGTTGATA |
| | ATATTAATTTTAAATCCCTAGTTGGATGTTTAGGTCCACACTGCGGTTCTTCTTCTCGTTCTTTTTT |
| | TGAAGATTTATTGTTTGACAAAGTTAAACTTTCAGATGTTGGTTTTGTTGAAGCTTATAACAATTGT |
| | ACTGGTGGTAGTGAAATTAGAGATCTTCTTTGTGTACAATCCTTTAATGGTATTAAAGTTTTGCCTC |
| | CTATTTTGTCTGAATCTCAAATTTCTGGTTACACCACAGCCGCTACTGTTGCTGCTATGTTTCCACC |
| | ATGGTCAGCAGCAGCTGGCATACCATTTTCTCTTAATGTACAATATAGAATTAATGGTTTGGGTGTT |
| | ACTATGGATGTTCTTAATAAAAATCAAAAGTTGATAGCTACTGCTTTTAATAATGCTCTTCTTTCTA |
| | TTCAGAATGGTTTTAGTGCTACCAACTCTGCACTTGCTAAAATACAAAGTGTTGTTAATTCTAATGC |
| | TCAAGCACTTAATAGTTTGTTACAGCAATTATTTAATAAATTTGGTGCAATTAGTTCTTCTTTACAA |
| | GAAATTTTATCTCGTCTCGATGCTTTAGAGGCTCAGGTTCAGATTGATAGGCTTATTAATGGTCGTT |
| | TAACTGCTTTAAATGCTTATGTCTCTCAACAGCTTAGTGATATTTCTCTTGTAAAATTTGGTGCTGC |
| | TTTAGCTATGGAGAAGGTTAATGAGTGTGTTAAAAGTCAATCTCCTCGTATTAATTTTTGTGGTAAT |
| | GGTAATCATATTTTGTCATTAGTTCAAAATGCTCCTTATGGTTTGTTGTTTATGCATTTTAGTTATA |
| | AACCTATTTCTTTTAAAACTGTTTTAGTAAGTCCTGGTTTGTGTATATCAGGTGATGTAGGTATTGC |
| | ACCTAAACAAGGGTATTTTATTAAACATAATGATCATTGGATGTTCACTGGTAGTTCTTACTATTAT |
| | CCTGAACCAATTTCAGATAAAAATGTTGTTTTTATGAATACTTGTTCTGTTAATTTTACTAAAGCGC |
| | CTCTTGTTTATTTGAATCATTCTGTACCAAAATTGTCTGATTTTGAATCTGAGTTATCTCATTGGTT |
| | TAAAAATCAAACATCCATTGCGCCTAATTTGACTTTAAATCTTCATACTATTAATGCTACTTTTTTA |
| | GATTTGTATTATGAGATGAATCTTATTCAAGAGTCTATTAAGTCTTTGAATAATAGTTATATCAATC |
| | TTAAAGATATAGGTACATATGAAATGTATGTAAAATGGCCTTGGTATGTTTGGCTACTAATTTCTTT |
| | TTCATTTATAATATTCCTTGTATTGCTCTTTTTTATATGTTGTTGTACTGGTTGTGGTTCTGCATGT |
| | TTTAGTAAATGTCATAATTGTTGTGATGAGTATGGTGGTCATCATGATTTTGTTATCAAAACATCTC |
| | ATGATGATTAGAATCTCTTGTCAGATCTCATTAAATCTAAACTTTATTTATGGACGTTTGGAGACCT |
| | AGCTACACACATTCTCTTGTTATTAGAGAATTTGGTGTTACAAACCTTGAAGATTTGTGTCTAAAGT |
| | ATAATTACTGTCAACCTATTGTTGGTTACTGTATTGTACCTTTAAATGTTTGGTGTCGCAAGTTTGG |
| | CAAATTTGCTTCTCACTTTACATTACGTAGTCACGATATTTCCCATAGTAATAATTTTGGTGTTGTA |
| | ACTAGTTTTACTACTTATGGTAATACTGTTTCTGAGGCTGTGTCTAGATTAGTTGAATCAGCTTCTG |
| | AATTTATTGTTTGGCGTGCAGAGGCACTTAATAAGTATGGTTGATTTATTTTTCAATGATACTGCTT |
| | GGTACATAGGACAGATTTTAGTTTTAGTTTTATTTTGTCTTATTTCTTTAATCTTTGTTGTTGCTTT |
| | TTTAGCAACTATTAAGCTTTGTATGCAACTTTGTGGTTTTTGTAATTTCTTTATTATTTCACCTTCG |
| | GCTTACGTTTATAAAAGAGGTATGCAGTTGTATAAGTCTTATAGTGAACAAGTTATACCACCCACTT |
| | CAGATTATTTAATCTAAATCTAAACATTATGAATAAATCTTTTCTTCCTCAATTTACTTCTGATCAA |
| | GCTGTTACATTCTTAAAAGAATGGAATTTCTCTTTGGGTGTAATACTACTTTTTATTACTATCATAT |
| | TGCAGTTCGGTTATACGAGCCGTAGTATGTTTGTTTATCTTATCAAGATGATTATTCTTTGGCTTAT |
| | GTGGCCATTGACTATCACCTTGACTATATTTAATTGTTTTTATGCTTTGAATAATGCTTTTCTTGCA |
| | TTTTCTATAGTGTTTACTATTATTTCTATTGTTATATGGATTCTTTATTTTGTTAATAGTATTCGGC |
| | TTTTTATTAGAACTGGCAGTTGGTGGAGTTTTAATCCAGAGACCAATAATCTTATGTGTATTGATAT |
| | GAAAGGCAAGATGTTTGTTAGGCCAGTTATTGAGGACTATCACACATTAACTGCTACTGTTATTCGT |
| | GGTCATCTTTATATACAGGGTGTCAAACTTGGCACTGGTTATACTCTTTCAGATTTGCCCGTATATG |
| | TTACTGTAGCTAAGGTGCAAGTACTTTGTACCTATAAACGTGCCTTTTTAGATAAGTTAGATGTTAA |
| | TAGTGGTTTTGCTGTTTTTGTTAAGTCTAAAGTTGGTAACTATCGTTTACCGTCTAGTAAACCTAGT |
| | GGTATGGATACTGCCTTGTTAAGAGCTTAAATCTAAACTATTAGGATGTCTTATACTCCCGGTCATT |
| | ATGCTGGAAGTAGAAGCTCCTCTGGAAATCGTTCAGGAATCCTCAAGAAAACTTCTTGGGCTGACCA |
| | ATCTGAGCGAAATTACCAAACCTTTAATAGAGGCAGAAAAACCCAACCTAAATTCACTGTGTCTACT |
| | CAACCACAAGGAAATACTATCCCACATTATTCCTGGTTCTCCGGGATCACTCAATTTCAAAAAGGTA |
| | GAGACTTTAAATTTTCAGATGGTCAAGGAGTTCCCATTGCTTTCGGAGTACCCCCTTCTGAAGCAAA |
| | AGGATATTGGTATAGACACAGCCGGCGTTCTTTTAAAACAGCTGATGGTCAACAAAAGCAGTTGTTA |
| | CCGAGATGGTATTTCTACTATCTCGGTACCGGCCCATATGCCAATGCATCCTATGGTGAATCCCTCG |
| | AAGGGGTCTTCTGGGTTGCTAATCACCAAGCTGACACTTCTACTCCCTCCGATGTTTCGTCAAGGGA |
| | TCCTACTACTCAAGAAGCTATCCCTACTAGGTTTCCGCCTGGTACGATTTTGCCTCAAGGCTATTAT |
| | GTTGAAGGCTCAGGAAGGTCTGCTTCTAATAGTCGACCAGGTTCACGTTCTCAATCACGTGGACCCA |
| | ATAATCGTTCATTAAGTAGAAGTAATTCTAATTTTAGACATTCAGATTCTATAGTAAAACCTGATAT |
| | GGCTGATGAGATCGCTAATCTTGTTTTAGCCAAGCTTGGTAAAGATTCTAAACCTCAGCAAGTCACT |
| | AAGCAAAATGCCAAGGAAATCAGGCATAAAATTTTAACAAAACCTCGCCAAAAGCGAACTCCTAATA |
| | AACATTGTAATGTTCAACAGTGTTTTGGTAAAAGAGGACCTTCTCAAAATTTTGGTAATGCTGAAAT |
| | GTTAAAGCTTGGTACTAATGATCCTCAGTTTCCTATTCTTGCAGAATTAGCTCCTACACCAGGTGCT |
| | TTTTTCTTTGGTTCTAAATTAGACTTGGTTAAAAGAGATTCCGAGGCTGACTCACCTGTTAAAGATG |
| | TTTTTGAACTTCATTATTCTGGTTCTATTAGGTTTGATAGTACTTTACCAGGCTTTGAGACAATTAT |
| | GAAAGTTCTTGAAGAGAATTTAAATGCTTACGTTAATTCTAATCAGAACACTGATTCTGATTCGTTG |
| | AGTTCTAAACCTCAGCGTAAAAGAGGTGTTAAACAATTACCAGAACAGTTTGACTCTCTTAATTTAA |
| | GTGCTGGTACTCAGCACATTTCAAATGATTTTACTCCTGAGGATCATAGTTTACTTGCTACTCTTGA |
| | TGATCCTTATGTAGAAGACTCTGTTGCTTAATGAGAATGAATCCTAATTCGACACTAGGTGGTAACC |
| | CCTCGCTATTATTCGGAATAGGACACTCTCTATCAGAATGAATTCTTGCTGTAATAACAGATAGAGT |
| | AGGTTGTTACAGACTATATATTAATTAGTAGAAATTTTATATTTAGACATTTGATTGTTAGAGTAGT |
| | TATAAGGTTTAGCTGTAGTATAAACGCCTCCGGGAAGAGCTATCAATTGTAGTGTTTAATATATATA |
| | TTAGTATATGATTGAAATTAATTATAGCCTTTTGGAGGAATTAC |
| 7 | GenBank Accession No. MN306036.1 (Human coronavirus OC43 strain |
| | HCoV_OC43/Seattle/USA/SC0682/2019, complete genome) |
| | ATCTCTTGTTAGATCTTTTTGTAATCTAAACTTTATAAAAACATCCACTCCCTGTAATCTATGCNTG |
| | TGGGCGTAGATTTTTCATAGTGGTGTTTATATTCATTTCTGCTGTTAACAGCTTTCAGCCAGGGACG |
| | TGTTGTATCCTAGGCAGTGGCCCTCCCATAGGTCACAATGTCGAAGATCAACAAATACGGTCTCGAA |
| | CTACACTGGGCTCCAGAATTTCCATGGATGTTTGAGGACGCAGAGGAGAAGTTGGACAACCCTAGTA |
| | GTTCAGAGGTGGATATGATTTGCTCCACCACTGCGCAAAAGCTGGAAACAGACGGAATTTGTCCTGA |
| | AAATCATGTGATGGTGGATTGTCGCCGACTTCTTAAACAAGAGTGTTGTGTGCAGTCTAGCCTAATA |
| | CGTGAAATTGTTATGAATGCAAGTCCATATTATTTGGAGGTGCTACTTCAAGATGCTTTGCAGTCCC |
| | GTGAAGCAGTTTTGGTTACAACCCCCTTAGGTATGTCCTTAGAGGCATGCTATGTGAGAGGTTGTAA |
| | TCCTAAAGGATGGACCATGGGTTTGTTTCGGCGTAGAAGTGTGTGTAACACTGGTCGTTGCACTGTT |
| | AATAAGCATGTGGCCTATCAGTTATATATGATTGATCCTACGGGTGTCTGTCTTGGTGCAGGTCAAT |
| | TCGTGGGTTGGGTCATACCCTTAGCCTTTATGCCTGTGCAATCCCGGAAATTTATTGTTCCATGGGT |
| | TATGTACTTGCGTAAGCGTGGCGAAAAGGGTGCTTATAATAAAGATCATGGATGTGGCGGTTTTGGA |
| | CATGTTTATGATTTTAAAGTTGAAGATGCTTATGACCAGGTGCATGATGAGCCTAAGGGTAAGTTTT |
| | CTAAGAAGGCTTATGCTTTAATTAGAGGGTATCGTGGTGTTAAACCACTTCTCTATGTAGACCAGTA |
| | TGGTTGTGATTATACTGGTAGTCTTGCAGATGGCTTAGAGGCTTATGCTGATAAGACATTGCAAGAA |
| | ATGAAGGCATTATTTCCTACTTGGAGTCAGGAACTCCCTTTTGATGTAATTGTGGCATGGCATGTTG |
| | TGCGTGATCCACGTTATGTTATGAGATTGCAGAGTGCTGCTACTATATGTAGTGTTGCATATGTTGC |
| | TAATCCTACTGAAGACTTGTGTGATGGTTCTGTTGTTATAAAAGAACCTGTGCATGTTTATGCAGAT |
| | GACTCTATTATTTTACGTCAATATAATTTATTTGACATTATGAGTCATTTTTATATGGAGGCAGATA |
| | CAGTTGTAAATGCTTTTTATGGTGTTGCTTTGAAAGATTGTGGTTTTGTTATGCAGTTTGGTTACAT |
| | TGATTGCGAACAAGACTTGTGTGATTTTAAAGGTTGGATTCCTGGTAACATGATAGATGGTTTTGCT |
| | TGCACCACTTGTGGTCATGTTTATGAAGTAGGTGATTTGATAGCACAATCTTCAGGTGTTTTGCCTG |
| | TTAACCCTGTATTGCATACTAAGAGTGCAGCAGGTTATGGTGGTTTTGGTTGTAAAGATTCTTTTAC |
| | TCTGTATGGCCAAACTGTAGTTTATTTTGGAGGTTGTGTGTATTGGAGTCCAGCACGTAATATATGG |
| | ATTCCTATATTAAAATCCTCTGTTAAGTCATATGACAGTTTGGTTTATACTGGAGTTTTAGGTTGCA |
| | AGGCTATTGTAAAGGAAACAAATCTCATTTGCAAAGCTCTGTACCTTGATTATGTTCAACACAAGTG |
| | TGGCAATTTACACCAACGGGAGTTGCTAGGTGTTTCAGATGTGTGGCATAAACAATTGCTAATAAAT |
| | AGAGGTGTTTATAAACCTCTGTTAGAGAATATTGATTATTTTAATATGCGACGCGCTAAATTTAGTT |
| | TAGAAACTTTTACTGTTTGTGCAGATGGCTTTATGCCTTTTCTTTTAGATGATTTAGTTCCACGCGC |
| | ATATTATTTGGCAGTAAGTGGTCAAGCATTTTGTGATTATGCAGATAAACTTTGCCATGCTGTTGTG |
| | TCTAAGAGTAAAGAGTTACTTGATGTGTCTCTGGATTCTTTAGGTGCAGCTATACATTATTTGAATT |
| | CTAAGATTGTTGATTTGGCTCAACATTTTAGTGATTTTGGAACAAGTTTTGTTTCTAAAATTGTTCA |
| | TTTCTTTAAGACTTTTACTACTAGCACTGCTCTTGCATTTGCATGGGTTTTATTTCATGTTTTGCAT |
| | GGTGCTTATATAGTAGTGGAGAGTGATATATATTTTGTTAAAAACATTCCTCGTTATGCTAGTGCTG |
| | TTGCACAAGCATTTCAGAGTGTTGCTAAAGTTGTACTGGACTCTTTAAGAGTTACTTTTATTGATGG |
| | CCTTTCTTGTTTTAAGATTGGACGTAGAAGAATTTGTCTTTCAGGCAGAAAAATTTATGAAGTTGCG |
| | CGTGGCTTGTTACATTCATCCCAATTGCCATTAGATGTTTATGATTTAACCATGCCTAGTCAAGTTC |
| | AGAAAGCCAAGCAAAAACCTATCTATTTAAAAGGTTCTGGTTCTGATTTTTCATTAGCGGATAGTGT |
| | AGTTGAAGTTGTTACAACTTCACTTACACCATGTGGCTATTCTGAACCACCTAAAGTTGCAGATAAA |
| | ATTTGCATTGTGGATAATGTTTATATGGCCAAGGCTGGTGATAAATATTACCCTGTCGTGGTTGATG |
| | ATCATGTTGGACTTTTGGATCAAGCATGGAGAGTTCCTTGTGCTGGACGGCGTGTTACATTTAAGGA |
| | ACAGCCTACAGTAAAGGAGATTATAAGCATGCCTAAGATTATTAAGGTTTTTTATGAGCTTGACAAT |
| | GATTTTAATACTATTTTAAATACTGCGTGTGGAGTGTTTGAAGTGGATGATACTGTCGATATGGAGG |
| | AATTTTATGCTGTGGTGATTGATGCCATAGAAGAGAAACTTTCTCCATGTAAGGAGCTTGAAGGTGT |
| | AGGTGCTAAAGTTAGTGCCTTTTTACAGAAATTAGAGGATAATCCCCTATTTTTATTTGATGAGGCT |
| | GGCGAAGAAGTTTTTGCTCCTAAATTGTATTGTGCCTTTACAGCTCCTGAAGATGATGACTTTCTTG |
| | AGGAAAGTGATGTTGAAGAAGATGATGTAGAAGGTGAGGAAACTGATTTAACTATCACAAGTGCTGG |
| | ACGGCCTTGTGTTGCTAGTGAACAGGAGGAATCTTCTGAAGTCTTAGAGGACACTTTGGATGATGGT |
| | CCAAGTGTGGAGACATCTGATTCACAAGTTGAAGAAGATGTAGAAATGTCGGATTTTGTTGATCTTG |
| | AATCTGTGATTCAGGATTATGAAAATGTTTGTTTTGAGTTTTATACTACAGAGCCAGAATTTGTTAA |
| | AGTTTTGGGTCTGTATGTGCCTAAAGCAACTCGCAACAATTGCTGGTTGCGATCAGTTTTGGCAGTG |
| | ATGCAGAAATTGCCCTGTCAATTTAAAGATAAAAATTTGCAGGATCTTTGGGTGTTATACAAGCAAC |
| | AGTATAGTCAGTTGTTTGTTGATACCTTGGTTAATAAGATACCTGCTAATATTGTACTTCCACAAGG |
| | TGGTTATGTTGCTGATTTTGCATATTGGTTTTTAACCTTATGTGATTGGCAGTGTGTTGCATACTGG |
| | AAATGCATTAAATGTGATTTAGCTCTTAAGCTTAAAGGCTTGGATGCTATGTTCTTTTATGGTGATG |
| | TTGTCTCACATATATGCAAGTGTGGTGAGTCTATGGTACTTATTGATGTTGATGTGCCATTTACAGT |
| | CCACTTTGCTCTTAAAGATAAGTTGTTTTGTGCATTTATTACTAAGCGTATTGTGTATAAAGCAGCT |
| | TGTGTTGTGGATGTTAATGATAGTCATTCTATGGCTGTTGTTGATGGTAAACAAATTGATGATCATC |
| | GTATCACTAGTATTACTAGTGATAAGTTTGATTTTATTATTGGGCATGGTATGTCATTTTCAATGAC |
| | TACTTTTGAAATTGCCCAATTGTATGGTTCTTGTATAACACCTAATGTGTGTTTTGTTAAAGGTGAT |
| | ATAATTAAAGTGTCTAAGCTTGTTAAAGCAGAAGTTGTTGTAAACCCTGCTAATGGCCATATGGTAC |
| | ATGGTGGTGGTGTTGCAAAAGCTATTGCAGTAGCAGCTGGACAGCAGTTTGTTAAAGAGACTACCAA |
| | TATGGTTAAGTCTAAAGGAGTTTGTGCTACTGGAGATTGTTATGTCTCTACAGGGGGCAAATTATGT |
| | AAAACTGTGCTTAATGTTGTTGGACCTGATGCGAGAACACAGGGTAAACAAAGTTATGTATTGTTAG |
| | AGCGTGTTTATAAACATTTTAACAACTATGACTGTGTTGTTACAACTTTGATCTCAGCTGGTATATT |
| | TAGTGTGCCTTCTGATGTGTCTTTAACATATCTACTTGGTACTGCTAAGAAACAAGTTGTTCTTGTT |
| | AGCAATAATCAAGAGGATTTTGATCTTATTTCTAAGTGTCAGATAACTGCTGTTGAGGGCACTAAGA |
| | AATTGGCAGCGCGTCTTTCCTTTAATGTTGGACGTTCCATTGTTTACGAAACAGATGCTAATAAGTT |
| | GATTTTAATCAATGATGTTGCATTTGTTTCGACATTTAATGTTTTACAGGATGTTTTATCCTTAAGA |
| | CATGATATAGCACTTGATGATGATGCACGAACCTTTGTTCAGAGCAATGTTGATGTTCTACCTGAGG |
| | GTTGGCGTGTTGTCAATAAGTTTTATCAAATTAATGGTGTTAGAACCGTTAAGTATTTTGAGTGTAC |
| | TGGAGGCATAGATATATGCAGCCAGGATAAAGTTTTTGGTTATGTACAGCAGGGTATTTTTAATAAG |
| | GCTACTGTTGCTCAAATTAAAGCCTTGTTTTTGGATAAAGTGGACATCTTGCTAACTGTTGATGGTG |
| | TTAATTTCACTAATAGGTTTGTGCCTGTTGGTGAAAGTTTTGGTAAGAGTCTAGGAAATGTGTTTTG |
| | TGATGGAGTTAATGTCACGAAGCATAAGTGTGATATAAATTATAAAGGTAAAGTCTTTTTCCAGTTT |
| | GATAATCTTTCTAGTGAAGATTTAAAGGCTGTAAGAAGTTCCTTTAATTTTGATCAGAAGGAATTGC |
| | TTGCCTATTACAACATGCTTGTTAATTGTTTTAAGTGGCAGGTTGTTGTTAATGGTAAGTATTTCAC |
| | TTTTAAGCAAGCTAATAACAATTGTTTTGTTAATGTTTCTTGCTTAATGCTCCAGAGCTTGCATCTG |
| | ACATTTAAAATTGTTCAATGGCAAGAGGCATGGCTTGAATTTCGTTCTGGCCGCCCTGCTAGATTTG |
| | TAGCTTTGGTTTTGGCCAAAGGTGGGTTTAAATTTGGAGATCCTGCTGATTCTAGAGATTTCTTGCG |
| | TGTTGTGTTTAGTCAAGTTGATTTGACTGGGGCAATATGTGATTTTGAAATTGCATGTAAATGTGGT |
| | GTAAAGCAGGAACAGCGTACTGGTCTAGATGCTGTTATGCATTTTGGTACATTGAGTCGTGAAGATC |
| | TTGAGATTGGTTATACCGTGGACTGTTCTTGCGGTAAAAAGCTAATTCATTGTGTACGATTTGATGT |
| | ACCATTTTTAATTTGCAGTAATACACCTGCTAGTGTAAAATTACCTAAGGGTGTAGGAAGTGCAAAT |
| | ATTTTTATAGGTGATAATGTTGGTCATTATGTTCATGTTAAGTGTGAACAATCTTATCAGCTTTATG |
| | ATGCTTCTAATGTTAAGAAGGTTACAGATGTTACTGGCAAATTGTCAGATTGTCTGTATCTTAAAAA |
| | TTTGAAACAAACTTTTAAATCTGTGTTAACCACCTATTATTNNNNNNNNNNNNNGAAAATTGAGTAT |
| | AAACCTGACTTGTCACAATACTATTGNNNNNNNNNNNNNNNNNNNNNNNNNNNNNNNATTAAAGCCC |
| | AATTTAAAACATTCGAGAAAGTAGATGGTGTGTATACTAATTTTAAATTGATAGGACACACCGTCTG |
| | TGACAGTCTTAATTCTAAGTTGGGTTTTGATAGCTCTAAAGAGTTTGTTGAATATAAGATTACTGAG |
| | TGGCCAACAGCTACAGGTGATGTGGTGTTGGCTAATGATGATTTGTATGTTAAGAGATATGAGAGGG |
| | GTTGTATTACTTTTGGTAAACCTGTTATATGGTTAAGCCATGAGAAAGCTTCTCTCAATTCTTTAAC |
| | ATATTTTAATAGACCTTTATTGGTTGATGATAATAAATTTGATGTTTTAAAAGTGGATGATGTTGAT |
| | GATAGTGGTGACAGCTCAGAGAGTGGTGCCAAAGAAACCAAAGAAATCAACATTATTAAGTTAAGTG |
| | GTGTTAAAAAACCATTTAAGGTTGAAGATAGTGTCATTGTTAACGATGATACTAGTGAAACCAAATA |
| | CGTTAAGAGTTTGTCTATTGTTGATGTGTATGATATGTGGCTTACAGGTTGTAAGTATGTTGTTAGA |
| | ACTGCTAATGCTTTGAGCAGAGCAGTTAACGTACCTACAATACGTAAGTTTATAAAATTTGGTATGA |
| | CTCTTGTTAGTATACCAATTGATTTGTTAAATTTAAGAGAGATTAAGCCTGCTGTTAATGTGGTTAA |
| | AGCTGTGCGAAATAAAACTTCTGCATGCTTTAATTTTATTAAATGGCTTTTTGTCTTATTATTTGGC |
| | TGGATTAAAATATCCGCTGATAATAAAGNAATCTACACCACAGAAATTGCATCAAAGCTTACGTGTA |
| | AGCTTGTAGCTTTAGCTTTTAAAAATGCATTTTTGACATTTAAGTGGAGTATGGTTGCTAGAGGTGC |
| | TTGCATTATAGCGACTATATTTCTATTGTGGTTTAATTTTATATATGCCAATGTAATTTTTAGTGAT |
| | TTTTATTTGCCTAAAATCGGTTTCTTGCCGACTTTTGTTGGTAAGATTGCACAGTGGATTAAGAACA |
| | CTTTTAGTCTTGTAACTATTTGTGATCTATATTCCATTCAGGATGTGGGTTTTAAGAATCAGTATTG |
| | TAATGGAAGTATTGCATGTCAGTTCTGCTTGGCAGGATTTGATATGTTAGATAATTATAAAGCCATT |
| | GATGTAGTACAGTATGAAGCTGATAGGAGAGCATTTGTTGATTATACAGGTGTGTTAAAGATTGTCA |
| | TTGAATTGATAGTAAGTTATGCCCTGTATACGGCATGGTTTTATCCATTGTTTGCCCTTATCAGTAT |
| | TCAGATTTTGACCACTTGGCTGCCTGAGCTTTTTATGCTTAGTACATTACATTGGAGTTTTAGGTTG |
| | TTGGTGGCTTTAGCTAATATGTTACCAGCACATGTGTTTATGAGGTTTTATATTATTATTGCCTCTT |
| | TTATTAAGCTCTTTAGCTTGTTTAAGCATGTTGCTTATGGTTGTAGTAAATCTGGTTGTTTGTTTTG |
| | TTACAAGAGGAATCGTAGTCTACGTGTTAAATGTAGTACTATCGTTGGTGGCATGATACGCTATTAC |
| | GATGTTATGGCTAATGGTGGCACTGGCTTTTGTTCAAAACATCAATGGAATTGCATTGATTGTGATT |
| | CTTATAAACCAGGTAATACTTTTATTACTGTTGAGGCCGCTCTTGATCTATCTAAGGAATTGAAACG |
| | GCCCATTCAGCCTACAGATGTTGCTTATCACACGGTTACTGATGTTAAGCAAGTTGGTTGTTCTATG |
| | CGCTTGTTCTATGATCGTGATGGACAGCGCATATATGATGATGTTAATGCTAGTTTGTTTGTGGATT |
| | NTAGTAATTTGCTACATTCTAAGGTTAAGAGTGTGCCTAATATGCATGTTGTGGTAGTGGAAAATGA |
| | TGCTGATAAAGCCAATTTTCTGAATGCTGCTGTATTTTATGCACAGTCTTTGTTTAGACCTATTTTA |
| | ATGGTTGATAAAAATCTGATAACTACTGCTAACACTGGTACGTCTGTTACAGAAACTATGTTTGATG |
| | TTTATGTGGATACATTTTTGTCTATGTTTGATGTGGATAAAAAGAGTCTTAATGCTTTAATAGCAAC |
| | TGCGCATTCTTCTATAAAACAGGGTACGCAGATTTATAAAGTTTTGGATACCTTTTTAAGCTGTGCT |
| | CGTAAAAGTTGTTCTATTGATTCAGATGTTGATACTAAGTGTTTAGCTGATTCTGTCATGTCTGCTG |
| | TATCGTCAGGTCTCGAATTGACGGATGAAAGTTGTAATAACTTGGTGCCAACATATTTGAAGAGTGA |
| | CAACATTGTAGCAGCTGATTTAGGTGTTCTGATTCAAAATTCTGCAAAGCATGTGCAGGGTAATGTT |
| | GCTAAAATAGCTGGTGTTTCCTGTATATGGTCTGTGGATGCTTTTAATCAGTTTAGTTCTGATTTCC |
| | AGCATAAATTGAAGAAAGCATGTTGTAAAACTGGTTTGAAACTGAAGCTTACTTATAATAAGCAGAT |
| | GGCTAATGTCTCTGTTTTAACTACACCCTTTAGTCTTAAAGGGGGTGCAGTTTTTAGTTATTTTGTT |
| | TATGTGTGTTTTGTGTTGAGTTTGGTCTGTTTTATTGGACTGTGGTGCTTAATGCCCACTTACACAG |
| | TACACAAATCAGATTTTCAGCTTCCTGTTTATGCCAGTTATAAAGTTTTAGATAATGGTGTTATTAG |
| | AGATGTTAGCGTTGAAGATGTTTGTTTCGCTAACAAATTTGAGCAATTTGATCAATGGTATGAGTCT |
| | ACATTTGGTCTAAGTTATTATAGTAACAGTATGGCTTGTCCCATTGTTGTTGCTGTAATAGATCAGG |
| | ATTTTGGCTCTACAGTGTTTAATGTCCCTACCAAAGTGTTACGATATGGTTATCATGTGTTGCACTT |
| | TATTACACATGCACTTTCTGCTGATGGAGTGCAGTGTTATACTCCACATAGTCAAATATCGTATTCT |
| | AATTTTTATGCTAGTGGCTGTGTGCTTTCCTCTGCTTGCACTATGTTTACAATGGCCGATGGTAGTC |
| | CACAACCTTATTGTTATACAGATGGGCTTATGCAAAATGCTTCTCTGTATAGTTCATTGGTACCTCA |
| | CGTGCGGTATAATCTTGCTAATGCTAAAGGTTTTATCCGTTTTCCAGAAGTGTTGCGAGAAGGGCTT |
| | GTACGTGTCGTGCGTACTCGTTCTATGTCGTATTGCAGAGTTGGATTATGTGAGGAAGCTGATGAGG |
| | GTATATGCTTTAATTTTAATGGTTCTTGGGTGCTTAATAATGATTATTATAGATCATTGCCTGGGAC |
| | CTTTTGTGGTAGAGATGTTTTTGATTTAATTTATCAGCTATTTAAAGGTTTAGCACAGCCTGTGGAT |
| | TTTTTGGCATTGACTGCTAGTTCCATTGCTGGTGCTATACTCGCTGTAATTGTTGTTTTGGTGTTTT |
| | ATTACCTAATAAAGCTTAAACGTGCTTTTGGTGATTACACCAGTGTTGTTTTTGTTAATGTGATTGT |
| | GTGGTGTGTAAATTTTATGATGCTTTTTGTGTTTCAAGTTTACCCCACACTTTCTTGTGTATATGCT |
| | ATTTGTTATTTTTATGCCACGCTTTATTTCCCTTCGGAGATAAGTGTGATAATGCACTTACAATGGC |
| | TAGTTATGTATGGCACTATTATGCCTTTATGGTTTTGTTTGCTATATATAGCTGTTGTTGTTTCAAA |
| | TCATGCTTTTTGGGTATTTTCTTACTGCAGAAAGCTTGGTACTTCTGTTCGTAGTGATGGTACATTT |
| | GAAGAAATGGCTCTCACTACTTTTATGATTACAAAAGATTCTTATTGTAAGCTTAAGAATTCTTTGT |
| | CTGATGTTGCTTTTAATNNNNNNNNNNNNNNGTATAATAAATATAGGTATTACAGCGGTAAAATGGA |
| | CACTGCTGCATATAGGGAGGCTGCTTGCTCTCAGTTGGCTAAAGCAATGGACACATTTACCAATAAT |
| | AATGGTAGTGATGTGCTTTACCAACCGCCTACTGCTTCCGTTTCAACTTCATTCTTGCAATCCGGTA |
| | TTGTGAAAATGGTAAATCCTACTTCTAAGGTAGAACCATGTGTTGTCAGTGTTACCTATGGTAATAT |
| | GACATTGAATGGTTTATGGTTGGATGACAAGGTCTACTGTCCCAGACATGTAATATGTTCTGCTTCA |
| | GATATGACTAATCCAGATTATACAAATTTGTTGTGTAGAGTAACATCAAGTGATTTTACTGTATTGT |
| | TTGATCGTTTAAGCCTTACAGTGATGTCTTATCAAATGCGGGGTTGTATGCTTGTTCTTACAGTGAC |
| | CCTGCAAAATTCTCGTACGCCAAAATATACATTTGGTGTGGTTAAACCTGGTGATACTTTTACTGTT |
| | TTAGCTGCTTATAACGGCAAACCACAAGGAGCCTTTCATGTAACTATGCGTAGTAGTTATACCATTA |
| | AGGGTTCCTTTTTATGCGGATCTTGTGGATCTGTTGGTTATGTAATAATGGGTGATTGTGTTAAATT |
| | TGTTTATATGCATCAATTGGAGCTTAGTACTGGTTGTCATACTGGTACTGACTTCAATGGGGATTTT |
| | TATGGTCCTTATAAGGATGCTCAGGTTGTTCAGTTGCCCATTCAGGATTATATACAATCTGTTAATT |
| | TTTTAGCATGGCTTTATGCTGCTATACTTAACAATTGTAATTGGTTTATACAAAGTGATAAGNNNNC |
| | TGTAGAAGATTTTAATGTGTGGGCTCTGTCCAATGGATTTAGCCAAGTTAAATCTGACCTTGTTATA |
| | GATGCTTTAGCTTCTATGACTGGTGTGTCTTTGGAAACACTGTTGGCTGCTATTAAGCGTCTTAAGA |
| | ATGGTTTCCAAGGACGTCAGATTATGGGTAGTTGCTCTTTTGAGGATGAATTGACACCTAGCGATGT |
| | TTATCAACAACTCGCTGGTATCAAGTTACAATCAAAACGCACTAGATTGTTTAAAGGTACTGTTTGT |
| | TGGATTATGGCTTCTACATTTTTGTTTAGTTGCATAATTACAGCATTTGTGAAATGGACTATGTTCA |
| | TGTATGTAACTACTAATATGTTTAGTATTACGTTTTGTGCACTTTGTGTTATAAGTTTGGCCATGTT |
| | GTTGGTTAAGCANNNNNNNNNNNNNNNNNNNATGTATATAACACCTGTGCTTTTTACATTGTTGTAT |
| | AACAACTATTTGGTTGTGTACAAGCATACATTTAGAGGCTATGTTTATGCATGGCTATCGTATTATG |
| | TTCCATCAGTTGAGTACACTTATACTGATGAAGTTATTTATGGCATGTTATTGCTTGTAGGAATGGT |
| | CTTTGTTACATTACGTAGCATTAACCATGATTTGTTTTCTTTTATAATGTTTGTTGGTCGTTTGATT |
| | TCTGTTTTCTCTTTGTGGTACAAGGGTTCTAACTTAGAGGAAGAAATTCTTCTTATGTTGGCTTCCC |
| | TTTTTGGTACTTACACATGGACAACAGTTTTATCTATGGCTGTAGCAAAGGTTATTGCTAAGTGGGT |
| | TGCTGTGAATGTCTTGTATTTCACAGATATACCTCAAATTAAGATAGTGCTTTTGTGCTATTTGTTT |
| | ATTGGTTATATTATTAGCTGTTATTGGGGTTTGTTTTCCTTGATGAACAGTTTGTTTAGAATGCCTT |
| | TGGGTGTTTATAATTATAAAATTTCAGTACAGGAATTAAGATATATGAATGCTAATGGATTGCGCCC |
| | TCCTAAGAATAGTTTTGAAGCCCTTATGCTTAATTTTAAGCTGTTGGGTATTGGAGGTGTTCCAATC |
| | ATTGAAGTATCTCAATTTCAATCAAAATTGACTGATGTCAAATGTGCTAATGTCGTCTTGCTTAATT |
| | GCTTGCAACATTTGCATGTTGCTTCTAATTCTAAGTTGTGGCATTATTGTAGCACTTTGCACAATGA |
| | AATACTTGCCACTTCGGATCTGAGTGTTGCTTTTGAAAAGCTTGCTCAGTTATTAATTGTTTTGTTT |
| | GCTAATCCAGCTGCTGTGGATAGCAAGTGCCTGACTAGTATTGAAGGAGTTTGCGATGATTACGCAA |
| | AGGACAATACTGTTTTGCAGGCTTTACAGAGTGAATTTGTTAATATGGCTAGCTTCGTTGAATATGA |
| | AGTTGCTAAGAAAAATCTTGATGAGGCGCGTTTTAGTGGTTCTGCTAATCAACAGCAGTTAAAACAG |
| | CTAGAGAAAGCCTGTAATATTGCTAAATCTGCTTATGAACGCGACCGTGCTGTAGCAAAAAAGTTGG |
| | AGCGTATGGCTGATTTAGCTCTTACTAATATGTATAAAGAAGCTAGAATTAATGATAAGAAGAGTAA |
| | GGTTGTTTCTGCCTTGCAAACTATGCTTTTTAGTATGGTGCGTAAGTTAGATAATCAAGCTCTGAAT |
| | TCAATATTAGATAACGCTGTGAAGGGTTGTGTACCATTGAATGCAATACCTTCATTGGCAGCAAATA |
| | CTCTGAATATAATTGTACCAGATAAAAGTGTTTATGACCAGATAGTTGATAATATCTATGTTACCTA |
| | TGCTGGTAATGTATGGCAGATTCAAACTATCCAGGATTCAGATGGTACAAATAAGCAGTTGAATGAG |
| | ATATCTGATGATTGTAACTGGCCACTAGTTATTATTGCAAATCGGTATAATGAGGTACCTGCTACTG |
| | TTTTGCAAAATAATGAATTAATGCCTGCTAAGTTGAAAATTCAGGTTGTTAATAGTGGTCCAGATCA |
| | GACTTGTAATACACCTACTCAATGTTACTATAATAATAGTAACAATGGTAAGATTGTTTATGCTATN |
| | NNNNNNNNNNNNNNNNGTCTTAAGTATACAAAAATTCTTAAAGATGATGGCAATTTTGTTGTTTTGG |
| | AGTTAGATCCTCCTTGTAAATTTACTGTTCAAGATGCTAAAGGTCTTAAAATTAAGTACCTTTATTT |
| | TGTAAAAGGTTGTAACACACTAGCAAGAGGCTGGGTTGTTGGTACAATTTCTTCTACAGTTAGATTG |
| | CAAGCTGGAACTGCTACTGAATATGCTTCCAACTCATCTATATTGTCTTTATGTGCGTTTTCTGTAG |
| | ATCCTAAGAAAACGTATTTAGATTTTATACAACAAGGAGGTACACCTATTGCCAATTGTGTTAAAAT |
| | GTTGTGTGACCATGCTGGTACCGGTATGGCCATTACTGTTAAACCCGATGCTACCACTAGTCAGGAT |
| | TCATATGGTGGTGCGTCTGTTTGTATATATTGCCGCGCACGAGTTGAACACCCAGATGTTGATGGGT |
| | TGTGCAAATTACGCGGCAAGTTTGTACAAGTGCCTGTAGGTATAAAAGATCCTGTGTCTTATGTTTT |
| | GACACATGATGTTTGTCGAGTTTGTGGATTTTGGCGGGATGGAAGTTGTTCATGTGTTAGCACTGAC |
| | ACTACTGTTCAATCAAAAGATACTAATTTTTTAAACGCGTTCGGGGTGCGAGTGTAGATGCCCGTCT |
| | CGTACCCTGCGCCAGTGGTTTATCTACTGATGTACAATTAAGGGCATTTGATATTTACAATGCTAGT |
| | GTTGCTGGCATTGGTTTACATTTAAAAGTTAATTGTTGCCGTTTTCAGCGTGTTGATGAGAACGGTG |
| | ATAAATTAGATCAGTTCTTTGTTGTTAAGAGGACAGATCTGACTATATATAATAGAGAGATGAAATG |
| | CTATGAGCGTGTAAAAGATTGTAAGTTTGTGGCTGAACACGATTTCTTTACATTTGATGTAGAAGGT |
| | AGTCGTGTGCCACACATTGTACGCAAGGATTTAACAAAGTATACTATGTTGGATCTTTGCTATGCAT |
| | TGCGACATTTTGATCGCAATGATTGCATGCTGCTTTGTGACATTCTCTCTATATATGCTGGTTGTGA |
| | ACAATCCTATTTTACTAAGAAGGATTGGTATGATTTTGTTGAAAATCCTGATATTATTAATGTGTAT |
| | AAAAAGCTAGGACCTATTTTTAATAGAGCCCTAGTTAGCGCTACTGAGTTTGCGGACAAATTGGTGG |
| | AGGTAGGCTTAGTAGGCGTTTTAACACTTGATAATCAAGATTTAAATGGTAAATGGTATGATTTTGG |
| | TGACTATGTTATTGCAGCCCCAGGATGTGGTGTTGCTATAGCAGATTCTTATTATTCTTATATCATG |
| | CCTATGCTGACCATGTGTCATGCATTGGATTGTGAATTGTATGTGAATAATGCTTATAGACTATTTG |
| | ATCTTGTACAGTATGATTTTACTGATTACAAGCTTGAATTGTTTAATAAGTATTTTAAGCACTGGAG |
| | TATGCCATATCATCCTAACACGGTTGATTGTCAGGATGATCGGTGTATTATACATTGTGCTAATTTT |
| | AACATACTTTTTAGTATGGTTTTACCTAATACATGTTTTGGGCCTCTTGTTAGGCAAATTTTTGTGG |
| | ATGGTGTGCCTTTTGTTGTTTCAATTGGCTACCATTATAAAGAACTTGGTATTGTGATGAATATGGA |
| | TGTGGATACACATCGTTATCGCTTGTCTTTAAAAGACTTGCTTTTATATGCTGCTGATCCAGCTTTG |
| | CATGTAGCTTCTGCTAGTGCATTGTATGATTTACGCACTTGCTGTTTTAGTGTTGCCGCTATAACAA |
| | GCGGTGTAAAATTTCAAACAGTTAAACCTGGTAATTTTAATCAGGATTTTTATGATTTTGTTTTAAG |
| | TAAAGGCCTGCTTAAAGAGGGTAGCCCAGTTGATCTGAAGCACTTTTTCTTTACACAGGATGGTAAT |
| | GCTGCTATTACTGATTATAATTATTATAAGTATAATTTGCCCACCATGGTGGACATTAAGCAGTTGT |
| | TGTTTGTTTTGGAAGTTGTTTATAAGTATTTTGAGATTTATGATGGTGGTTGTATACCGGCATCACA |
| | AGTCATTGTTAATAATTATGATAAGAGTGCTGGCTATCCATTTAACAAATTTGGAAAAGCCAGGCTC |
| | TATTATGAAGCATTATCATTTGAGGAACAGGATGAAATTTACGCTTATACTAAGCGTAATGTCCTGC |
| | CAACACTTACTCAAATGAATTTGAAATATGCTATTAGTGCTAAGAATAGAGCCCGCACTGTTGCTGG |
| | TGTTTCCATACTTAGTACTATGACTGGCAGAATGTTTCATCAAAAATGTTTGAAAAGTATAGCAGCT |
| | ACACGTGGTGTTCCTGTAGTTATAGGCACCACTAAATTTTATGGTGGCTGGGATGATATGTTACGCC |
| | GCCTTATTAAAGATGTCGACAATCCTGTTCTTATGGGTTGGGATTATCCTAAGTGTGATCGTGCTAT |
| | GCCAAACATACTACGTATTGTTAGTAGTTTGGTATTAGCCCGAAAACATGAGACATGTTGTTCGCAA |
| | AGCGATAGGTTTTATCGACTTGCGAATGAATGCGCACAAGTTTTGAGTGAAATTGTTATGTGTGGTG |
| | GCTGTTATTATGTTAAGCCTGGTGGCACTAGTAGTGGTGATGCAACTACTGCTTTTGCTAATTCAGT |
| | CTTTAACATATGTCAAGCTGTTTCAGCCAATGTATGTGCCTTAATGTCATGCAATGGCAATAAGATT |
| | GAAGATCTTAGTATACGTGCTCTTCAGAAGCGCTTATACTCACATGTGTATAGAAGTGATAAGGTTG |
| | ATTCAACCTTTGTCACAGAATATTATGAATTTTTAAATAAGCATTTTAGTATGATGATTTTGAGTGA |
| | TGATGGGGTTGTGTGTNNNNNNNNNNNNNNNNNNNNNNNNNNNNNNNNNNNNNNNNNNNNNNNNNNN |
| | NNNNNGGTATTATATTATCAAAATAACGTTTTTATGTCAGAATCCAAATGTTGGGTTGAGCATGACA |
| | TAAATAATGGACCTCATGAATTTTGTTCACAACACACAATGCTTGTAAAGATGGATGGTGACGATGT |
| | CTACCTTCCATATCCTAATCCTAGTCGTATATTAGGAGCTGGATGTTTTGTAGATGATTTGTTAAAG |
| | ACTGATAGTGTTCTTTTAATAGAACGATTTGTAAGTCTTGCAATAGATGCTTATCCACTTGTGTATC |
| | ATGAAAATGAAGAATACCAAAAGGTTTTTCGTGTTTATTTGGCGTATATAAAGAAGTTGTACAATGA |
| | CCTGGGTAATCAGATCTTGGATAGCTACAGTGTTATTTTAAGTACTTGTGATGGACAAAAGTTCACT |
| | GATGAATCCTTTTACAAGAACATGTATTTAAGAAGTGCAGTCATGCAGAGTGTTGGAGCTTGCGTGG |
| | TCTGCTCTTCTCAAACATCATTACGTTGTGGCAGTTGCATCAGAAAGCCTCTTCTTTGCTGCAAGTG |
| | TTGTTATGATCACGTTATGGCGACTGATCATAAATATGTCTTGAGTGTTTCACCATATGTGTGTAAT |
| | GCACCAGGATGTGATGTAAATGATGTTACCAAATTGTATCTAGGTGGTATGTCATATTATTGTGAAG |
| | ACCATAAGCCACAATATTCATTCAAGTTGGTAATGAATGGTTTGGTTTTTGGTCTATATAAACAATC |
| | TTGTACAGGATCTCCGTACATAGACGATTTTAATCGTATAGCTAGTTGTAAATGGACCGATGTGGAT |
| | GATTACATACTAGCTAATGAATGTACAGAGCGCTTGAAATTGTTTGCTGCAGAAACGCAAAAGGCAA |
| | CCGAGGAAGCCTTTAAGCAGAGTTATGCATCAGCAACAATACAAGAGATTGTTAGTGAGCGCGAATT |
| | GATTCTCTCTTGGGAGATTGGAAAAGTTAAGCCACCACTTAATAAAAATTATGTTTTTACTGGCTAC |
| | CATTTTACTAAAAATGGTAAGACAGTTTTAGGTGAGTATGTTTTTGATAAGAGTGAGTTGACTAATG |
| | GTGTGTACTATCGCGCCACAACCACTTATAAGCTATCTGTAGGAGATGTTTTTGTTTTAACCTCTCA |
| | TTCAGTAGCTAATTTAAGTGCTCCTACGCTTGTTCCGCAGGAGAATTATAGTAGTATTAGATTTGCT |
| | AGTGTTTATAGTGTGCTTGAGACGTTTCAGAACAATGTTGTTAATTATCAACACATTGGTATGAAAC |
| | GTTACTGTACCGTGCAAGGACCTCCTGGTACAGGGAAGTCACATCTTGCTATTGGTCTTGCTGTATT |
| | TTATTGTACAGCACGTGTTGTATACACAGCGGCCAGCCATGCAGCTGTTGACGCATTGTGTGAAAAA |
| | GCATATAAATTTTTGAATATAAATGATTGCACTCGTATTGTTCCGGCCAAGGTCAGGGTAGAGTGCT |
| | ATGATAAGTTTAAAATTAATGACACCACTCGTAAGTATGTGTTTACTACCATAAATGCATTACCTGA |
| | GATGGTGACTGATATTGTTGTTGTAGATGAAGTTAGCATGCTTACCAATTATGAGCTTTCTGTTATT |
| | AATGCTCGTATTCGTGCTAAGCATTATGTTTATATTGGTGATCCTGCTCAATTGCCAGCACCACGTG |
| | TGTTATTGAGCAAGGGTACACTTGAACCTAAATATTTTAACACTGTTACTAAGCTCATGTGTTGCTT |
| | AGGGCCAGACATTTTTCTTGGTACATGTTATAGATGTCCTAAGGAAATTGTTGATACAGTGTCCGCC |
| | TTGGTTTATGAAAATAAGCTTAAGGCTAAGAATGAGAGTAGTTCATTGTGTTTTAAGGTCTATTATA |
| | AGGGCGTTACAACACATGAAAGTTCTAGTGCTGTAAATATGCAGCAGATTTATTTGATTAATAAGTT |
| | TTTGAAGGCTAACCCTTTGTGGCATAAAGCTGTTTTTATTAGCCCATATAATAGTCAGAACTTTGCA |
| | GCTAAGCGTGTTTTGGGTTTACAAACCCAAACCGTGGATTCTGCTCAAGGTTCTGAATACGATTATG |
| | TTATATATTCACAGACTGCAGAAACAGCGCATTCTGTAAATGTTAATCGCTTCAATGTTGCCATTAC |
| | TCGAGCCAAGAAAGGTATTCTTTGTGTTATGAGTAATATGCAGTTGTTTGAAGCATTACAGTTTACT |
| | ACATTGACCTTAGATAAAGTGCCACAGGCCGTTGAAACTAAAGTTCAATGTAGTACTAATTTATTTA |
| | AAGATTGTAGCAAGAGTTATAGCGGTTACCACCCAGCTCATGCTCCTTCATTTTTGGCAGTAGATGA |
| | CAAATATAAGGCAACTGGCGATTTAGCCGTGTGTCTTGGCATTAGTGATTCTGCTGTTACATATTCA |
| | AGATTAATATCACTCATGGGTTTTAAATTGGATGTTACCCTTGATGGGTATTGTAAGCTTTTTATAA |
| | CTAAAGAAGAAGCTGTTAAACGCGTGCGTGCCTGGGTTGGCTTTGATGCTGAAGGTGCTCATGCCAC |
| | GCTTGATAGCATTGGGACAAATTTCCCACTCCAATTAGGATTTTCCACAGGAATTGATTTTGTTGTG |
| | GAAGCCACTGGTTTGTTTGCTGATAGAGATGGTTACAGCTTTAAAAAGGCTGTGGCGAAAGCTCCTC |
| | CTGGTGAACAATTTAAGCACCTCATCCCTTTGATGACGAGAGGTCATCGCTGGGATGTTGTTAGACC |
| | TAGAATAGTACAAATGTTTGCAGATCATTTAATTGATCTGTCTGATTGTGTTGTGCTAGTTACATGG |
| | GCAGCCAACTTTGAGCTCACTTGTCTCCGCTACTTTGCAAAAGTAGGTCGTGAGATTTCTTGTAATG |
| | TATGCACTAAACGTGCTACAGTTTACAATTCTAGAACTGGTTACTATGGTTGTTGGCGCCATAGTGT |
| | TACATGTGATTACTTGTATAATCCACTTATTGTTGATATTCAACAGTGGGGATATATTGGTTCTTTA |
| | TCAAGTAATCANGATTTATATTGTAGTGTCCATAAAGGAGCACATGTTGCTTCCTCTGATGCTATAA |
| | TGACACGGTGTTTGGCCGTTTATGATTGCTTTTGCAATAATATTAATTGGAATGTGGAGTATCCCAT |
| | CATTTCAAATGAGTTAAGTATTAATACCTCTTGTAGAGTCTTGCAGCGTGTGATTCTTAAAGCTGCC |
| | ATGCTCTGCAACAGATATACTTTGTGTTATGATATTGGCAACCCAAAAGGGATTGCCTGTGTCAAAG |
| | ATTTTGATTTTAAGTTCTATGATGCCCAACCAATTGTTAAGTCTGTTAAGACTCTTTTGTATTCTTT |
| | TGAGGCACATAAGGACTCTTTTAAAGACGGTTTGTGTATGTTTTGGAACTGTAATGTGGATAAGTAT |
| | CCACCGAATGCAGTTGTATGTAGATTTGACACTAGAGTGTTGAATAATTTAAATCTTCCTGGCTGTA |
| | ATGGAGGTAGTTTGTATGTTAATAAACATGCATTCCACACTAAACCCTTTGCTAGGGCAGCCTTTGA |
| | GCATTTGAAGCCTATGCCATTCTTCTATTATTCAGATACGCCTTGTGTGTATATGGATGGTATGGAT |
| | GCTAAGCAGGTTGATTATGTACCTTTGAAATCTGCCACGTGCATCACAAGATGCAATTTAGGTGGTG |
| | CAGTTTGTTTAAAACATGCTGAAGAGTATCGTGAGTACTTAGAGTCTTACAATACAGCTACTACAGC |
| | AGGTTTTACTTTTTGGGTCTATAAGACATTTGATTTTTATAATTTGTGGAATACGTTCACCAAGCTA |
| | CAAAGCTTGGAGAATGTTGTATATAATTTAGTCAAGACTGGTCATTATACAGGACAGGCTGGTGAAA |
| | TGCCTTGTGCCATTATAAATGATAAAGTTGTGACTAAGATCGATAAGGAGGATGTTGTCATTTTTAT |
| | TAATAATACAACATACCCTACTAATGTGGCTGTTGAATTATTTGCCAAGCGCAGTGTTCGACACCAC |
| | CCAGAGCTTAAGCTCTTTAGAAATTTAAATATAGACGTGTGTTGGAAGCACGTCATTTGGGATTATG |
| | CTAGAGAAAGTATATTTTGCAGTAATACCTATGGTGTCTGCATGTATACAGATTTAAAGTTCATTGA |
| | TAAATTGAATGTCCTTTTTGATGGTCGTGATAATGGTGCTTTTGAAGCTTTTAAACGCTCTAATAAT |
| | GGCGTTTACATTTCCACGACAAAAGTTAAGAGTCTTTCGATGATAAGAGGTCCACCGCGTGCTGAAT |
| | TAAATGGCGTGGTGGTGGACAAGGTTGGAGACACTGATTGTGTGTTTTATTTTGCTGTGCGTAAAGA |
| | AGGTCAGGATGTCATCTTCAGCCAATTCGACAGCCTGGGAGTCAGCTCTAACCAGAGCCCACAAGGT |
| | AATCTGGGGAGTAATGGTAAACCCGGTAATGTCGGTGGTAATGATGCTCTGTCAACCTCAACTATCT |
| | TTACACAAAGCCGTGTTATTAGCTCTTTTACATGTCGTACTGATATGGAAAAAGATTTTATAGCTTT |
| | AGATCAAGATGTGTTTATTCAGAAGTATGGTTTGGAGGACTATGCCTTTGAACACATTGTTTATGGT |
| | AATTTCAACCAGAAGATTATTGGTGGTTTGCACTTGTTAATAGGCTTGTACCGAAGACAGCAAACTT |
| | CCAATCTGGTTGTTCAGGAGTTTGTCTCATATGACTCCAGCATACACTCTTATTTTATCACTGACGA |
| | GAAGAGTGGTGGTAGTAAGAGTGTTTGCACTGTTATAGACATTTTATTGGATGATTTTGTGACTCTT |
| | GTTAAGTCACTTAATCTTAATTGTGTGAGTAAAGTTGTTAATGTTAATGTTGATTTTAAAGATTTTC |
| | AGTTTATGCTTTGGTGTAACGATGAGAAAGTTATGACTTTCTATCCTCGTTTGCAAGCTGCATCTGA |
| | CTGGAAGCCTGGTTATTCTATGCCTGTATTATATAAGTATTTGAATTCTCCAATGGAAAGAGTTAGT |
| | CTCTGGAATTATGGGAAGCCAGTTACTTTGCCTACAGGCTGTATGATGAATGTTGCTAAGTATACTC |
| | AGTTATGTCAATATCTGAATACTACAACATTAGCTGTACCTGTTAATATGCGAGTTTTGCATTTAGG |
| | TGCAGGTTCAGAAAAAGGAGTAGCACCGGGTTCTGCAGTTCTTAGGCAGTGGTTGCCTGCTGGTACT |
| | ATTCTTGTAGATAATGATTTATACCCATTTGTTAGTGACAGTGTCGCTACATATTTTGGGGATTGTA |
| | TAACTTTACCCTTTGATTGTCAATGGGATTTGATAATTTCTGATATGTATGACCCTATTACTAAGAA |
| | CATAGGTGAGTACAATGTGAGTAAAGATGGTTTCTTTACATACATTTGTCATATGATTCGAGACAAG |
| | TTAGCTCTTGGTGGCAGTGTTGCTATAAAAATAACAGAGTTTTCTTGGAATGCAGAATTATATAATT |
| | TAATGGGGTATTTTGCATTTTGGACTGTGTTTTGCACAAATGCAAATGCTTCTTCTAGTGAAGGATT |
| | TTTAATTGGCATAAATTATTTGTGTAAGCCCAAGGTTGAGATAGATGGAAATGTTATGCATGCCAAT |
| | TATTTGTTTTGGAGAAATTCCACAGTTTGGAACGGGGGTGCTTATAGCCTGTTTGACATGGCTAAAT |
| | TCCCGCTTAAGTTGGCTGGTACTGCCGTAATAAATTTAAGAGCAGACCAGATTAATGATATGGTTTA |
| | TTCCCTTCTTGAAAAGGGTAAACTACTTATTAGAGATACAAATAAAGAAGTTTTCGTTGGTGACAGT |
| | TTGGTTAATGTAATCTAAACTTTAAAAATGGCTGTCGCTTATGCAGACAAGCCTAATCATTTTATCA |
| | ATTTTCCACTTACCCATTTTCAGGGTTTTGTGTTAAATTATAAAGGTTTACAATTTCAAATTCTCGA |
| | TGAAGGAGTGGATTGTAAAATACAAACAGCGCCACACATTAGTCTTACTATGCTGGATATACAGCCT |
| | GAAGACTATAAAAGTGTTGATGTCGCTATTCAAGAAGTTATTGATGATATGCATTGGGGTGATGGTT |
| | TTCAGATTAAATTTGAGAATCCTCACATCCTAGGAACATGCATAGTTTTAGATGTTAAAGGTGTAGA |
| | AGAATTGCATGACGATTTAGTTAATTACATTCGTGATAAAGGTTGTGTTGCTGACCAATCCAGGAAA |
| | TGGATTGGCCATTGTACCATAGCTCAACTCACGGATGCAGCACTGTCCATTAAGGAAAATGTTGATT |
| | TTATAAACAGCATGCAATTCAATTATAAAATCACCATCAACCCCTCATCACCAGCTAGACTTGAAAT |
| | AGTTAAGCTCGGTGCTGAAAAGAAAGATGGTTTTTATGAAACCATAGTTAGTCACTGGATGGGAATT |
| | CGTTTTGAATACACATCGCCCACTGATAAGCTAGCTATGATTATGGGTTATTGTTGTTTAGATGTGG |
| | TACGTAAAGAGCTAGAAGAAGGCGATCTTCCCGAGAATGATGATGATGCTTGGTTTAAGCTATCGTA |
| | CCATTATGAAAACAATTCTTGGTTCTTCCGACATGTCTACAGGAAAAGTTTTCATTTCCGTAAGGCT |
| | TGTCAAAATTTAGATTGTAATTGTTTGGGGTTTTATGAATCTTCAGTTGAAGAATATTAAACTCAGT |
| | GAAAATGTTTTTGCTTTCTAGATTTATTCTAGTTAGCTGCGTAATTGGTAGCTTAGGTTTTTATAAC |
| | CCTCCTACCAATGTTGTTTCGCATGTAAATGGAGATTGGTTTTTATTTGGTGACAGTCGTTCAGATT |
| | GTAATCATATTGTTAATATCAACCCCCATAATTATTCTTATATGGACCTTAATCCTGCTTTATGTGA |
| | TTCTGGTAAAATATCATCTAAAGCTGGCAACTCCATTTTCAGGAGTTTTCACTTTACCGATTTTTAT |
| | AATTATACAGGCGAAGGTCAACAAATTATTTTTTATGAGGGTGTTAATTTTACGCCTTATCATGCCT |
| | TTAAATGCAACCGTTCTGGTAGTAATGATATTTGGATGCAGAATAAAGGCTTGTTTTATACTCAGGT |
| | TTATAAGAATATGGCTGTGTATCGCAGCCTTACTTTTGTTAATGTATCATATGTTTATAATGGCTCC |
| | GCACAAGCTACATCTTTTTGTAAATCTGGTAGTAAGCTTAAGAATTTAGTCCTTAATAACCCTGCTT |
| | ATATAGCTCCTCAAGCTAAATCTGGGATTATTACGAAGGTTGAAGCTGATTTTTATTTGTCAGGTTG |
| | TGACGAGTATATCGTACCACTTTGTATTTTTAACGGCAAGTCTTTGTCGAATACAAAGTATTATGAT |
| | CATAGTCAATATTATTTTAATAAAGACACTGGTGTTATTTATGGTCTCAATTCAACAGAAACCATTA |
| | CCACTGGTTTTGATCTTAATTGTCATTATTTAGTTTTACCCTCTGGTAATTATTTAGCCATTTCAAA |
| | TGAGCTATTGTTAACTGTTCCTACGAAAGCAATCTGTTTTAATAAGCGTAAGGATTTTACGCCTGTA |
| | CAGGTTGTTGATTCGCGGTGGAACAATGCCAGGCAGTCTGATAACATGACGGCGGTTGCTTGTCAAC |
| | CTCCGTACTGTTATTTTCGTAATTCTACTACCAACTATGTTGGTGTTTATGATATTAATCATGGAGA |
| | TGCTGGTTTCACTAGCATACTCAGTGGTTTGTTATATAATTCATCTTGTTTTTCGCAGCAAGGTGTT |
| | TTTAGGTATGATAATATTAGCAGTGTTTGGCCTCTCTACCCCTATGGCAGATGTCCTACTGCTGCTT |
| | ATATTAATATCCCTGATTTACCCATTTGTGTGTATGATCCGCTACCAGTTATTTTGCTTGGCATTCT |
| | TTTGGGCGTTGCCATTGTAATTATTGTAGTTTTTTTGTTACATTTTATGGTGGATAATGTTACTAGG |
| | CTGCATGATGCTTAGACCATAATCTAAACATGTTTTTGATACTTTTAATTTCCTTACCAACGGCTTT |
| | TGCTGTTATAGGAGATTTAAATTGTACTTTAGATCCTAGGCTTAAAGGTAGCTTTAATAATAGAGAC |
| | ACCGGTCCTCCTTCTATAAGTATTGATACTGTTGATGTTACTAATGGTTTGGGTACTTATTATGTTT |
| | TAGATCGTGTGTATTTAAATACCACGTTGTTTCTTAATGGTTATTACCCCACTTCAGGTTCCACATA |
| | TCGTAATATGGCACTGAAGGGAACTGACTTATTGAGCACATTATGGTTTAAACCACCATTTCTTTCT |
| | GATTTTATTAATGGTATTTTTGCTAAGGTCAAAAATACCAAGGTTTTTAAAGATGGTGTAATGTATA |
| | GTGAGTTCCCTGCTATAACTATAGGTAGTACTTTTGTAAATACATCCTATAGTGTGGTAGTACAACC |
| | ACGTACAATTAATTCAACACAGGATGGTGTTAATAAATTACAAGGTCTTTTAGAGGTCTCTGTTTGC |
| | CAGTATAATATGTGTGAGTACCCACATACGATTTGTCATCCTAACTTGGGTAACCATTTCAAAGAAC |
| | TATGGCATTATGATACAGGTGTTGTTTCCTGTTTGTATAAGCGTAATTTCACATATGATGTGAATGC |
| | TACTTATTTGTATTTTCATTTTTATCAAGAAGGTGGTACTTTTTATGCATATTTTACAGACACTGGT |
| | TTTGTTACTAAGTTTTTGTTTAATGTTTATTTAGGCATGGCACTTTCACACTATTATGTCATGCCTC |
| | TGACTTGTATTCGTAGGTCTAAAGACGGTTTTAGTCCAGAATATTGGGTTACACCCCTCACTCCTAG |
| | ACAATATTTACTCGCTTTCAATCAAGATGGTATTATTTTTAATGCTGTTGATTGTATGAGTGATTTT |
| | ATGAGTGAGATTAAGTGTAAAACACAATCTATAGCACCACCCACTGGTGTTTATGAATTAAACGGTT |
| | ACACTGTTCAGCCAATTGCAGATGTTTACCGACGCAAACCTGATCTTCCCAATTGTAATATAGAAGC |
| | TTGGCTTAATGATAAGTCGGTGCCCTCCCCATTAAATTGGGAACGTAAGACATTTTCAAATTGTAAT |
| | TTTAATATGAGCAGCCTGATGTCTTTTATTCAGGCAGATTCATTTACTTGTAATAATATTGATGCTG |
| | CTAAGATATATGGTATGTGTTTTTCCAGCATAACTATAGATAAGTTTGCTATACCCAATCGCAGGAA |
| | GGTTGACCTACAATTGGGTAATTTGGGCTATTTGCAGTCATCCAACTATAGAATTGATACTACTGCA |
| | ACAAGTTGTCAGTTGTATTATAATTTACCTGCTGCTAATGTCTCTGTTAGCAGGTTTAATCCTTCTA |
| | CTTGGAATAAGAGATTTGGTTTTATAGAAGATTCTGTTTTTAAACCTCAACCTGCAGGTGTTCTTAC |
| | CAATCATGATGTAGTTTATGCCCAACATTGTTTTAAAGCTCCTAAAAATTTCTGTCCGTGTAGTTCC |
| | TGTTCTGGTAAAAATAATGGTATAGGCACTTGTCCTGCAGGTACTAATTCTTTAACTTGTGATAATT |
| | TATGTACTCTTGATCCTATTACACTTAAAGCTCCAGATACTTATAAGTGTCCCCAATCTAAATCTTT |
| | AGTTGGTATAGGTGAGCACTGTTCGGGTCTTGCTGTTAAAAGTGATTATTGTGGAAACAATTCTTGT |
| | ACTTGCCAACCACAAGCATTTTTGGGTTGGTCTGCAGACTCTTGTTTACAAGGAGACAAGTGTAATA |
| | TTTTTGCTAATTTCATTTTGCATGATGTTAATAATGGTCTTACTTGTTCCACTGATTTACAAAAAGC |
| | TAACACAGAAATAGAACTTGGTGTTTGTGTTAATTATNACCTCTATGGTATTTCAGGCCAAGGCATT |
| | TTTGTTGAGGTTAATGCGACTTATTATAATAGTTGGCAGAACCTTTTATATGATTCTAATGGTAACC |
| | TCTACGGTTTTAGAGACTACATAACAAACAGAACTTTTATGATTCATAGTTGCTATAGCGGTCGTGT |
| | TTCTGCGGCCTATCATGCCAATTCTTCCGAACCAGCATTGCTATTTCGGAATATTAAATGCAACTAC |
| | GTTTTTAATAATAGTCTTACACGACAGCTGCAACCCATTAACTATTCGTTTGATAGTTATCTTGGTT |
| | GTGTTGTCAATGCTTATAATAGTACTGCTATTTCTGTCCAAACATGTGATCTCACAGTAGGTAGTGG |
| | TTACTGTGTGGATTACTTTAAAAACAGACGAAGTCGTAGAGCGATCACCACTGGTTATCGGTTTACT |
| | AATTTTGAGCCATTTACTGTTAATTCAGTAAATGATAGTTTGGAACCTGTAGGTGGTTTGTATGAAA |
| | TTCAAATACCTTCAGAGTTTACTATAGGTAATATGGAGGAGTTTATTCAAACAAGCTCTCCCAAAGT |
| | TACTATTGATTGTGCTGCATTTGTCTGTGGTGATTATGCAGCATGTAAATTACAGTTGGTTGAATAT |
| | GGTAGTTTCTGTGATAACATTAATGCCATACTCACAGAAGTAAATGAACTACTTGATACTACACAGT |
| | TGCAAGTAGCTAATAGTTTAATGAATGGTGTTACTCTTAGCACTAAGCTTAAAGATGGTGTTAATTT |
| | CAATGTAGACGACATCAATTTTGCCCCTGTATTAGGTTGTCTAGGCAGCGAATGTAGTAAAGCTTCC |
| | AGTAGATCTGCTATAGAGGATTTACTTTTTGATAAAGTAAAGTTATCTGATGTCGGTTTCGTTGAGG |
| | CTTATAATAATTGTACAGGAGGTGCCGAAATTAGGGACCTCATTTGTGTGCAAAGTTATAAAGGCAT |
| | CAAAGTGTTACCTCCACTGCTCTCAGAAAATCAGATCAGTGGATATACTTTGGCTGCCACCTCTGCT |
| | AGTCTATTTCCTCCTTGGACAGCAGCAGCAGGTGTACCATTTTATTTAAATGTTCAGTATCGCATTA |
| | ATGGGCTTGGTGTCACCATGGATGTGCTAAGTCAAAATCAAAAGCTTATTGCTAATGCATTTAACAA |
| | TGCCCTTCATGCTATTCAGCAAGGGTTCGATGCAACTAATTCTGCTTTAGTTAAAATTCAAGCTGTT |
| | GTTAATGCAAATTCTGAAGCTCTTAATAACTTATTGCAACAACTCTCTAATAGATTTGGTGCTATAA |
| | GTGCTTCTTTACAAGAAATTCTATCTAGACTTGATGCTCTTGAAGCGGAAGCTCAGATAGATAGACT |
| | TATTAATGGTCGTCTCACCGCTCTTAATGCTTATGTTTCTCAACAGCTTAGTGATTCTACACTGGTA |
| | AAATTTAGTGCAGCACAAGCTATGGAGAAGGTTAATGAATGTGTCAAAAGCCAATCATCTAGGATAA |
| | ATTTCTGTGGTAATGGTAATCATATTATATCATTAGTGCAGAATGCTCCATATGGTTTGTATTTTAT |
| | CCACTTTAATTATGTCCCTACTAAGTATGTCACAGCGAAGGTTAGTCCTGGTCTGTGCATTGCTGGT |
| | AATAGAGGTATAGCTCCTAAGAGTGGTTATTTTGTTAATGTAAATAATACTTGGATGTACACTGGTA |
| | GTGGTTATTACTACCCTGAACCTATAACTGAAAATAATGTTGTTGTTATGAGTACCTGCGCTGTTAA |
| | TTATACTAAAGCGCCGTATGTAATGCTGAACACTTCAATACCCAACCTTCCTGATTTTAAGGAAGAG |
| | TTGGACCAATGGTTCAAAAATCAAACATCAGTGGCACCAGATTTGTCACTTGATTATATAAATGTTA |
| | CATTCTTGGACCTACAAGTTGAAATGAATAGGTTACAGGAGGCAATAAAAGTTTTAAATCATAGCTA |
| | CATCAATCTCAAGGACATTGGTACATATGAATATTATGTAAAATGGCCTTGGTATGTATGGCTTTTA |
| | ATCTGCCTTGCTGGTGTAGCTATGCTTGTTTTACTATTCTTCATATGCTGTTGTACAGGATGTGGGA |
| | CTAGTTGTTTTAAGAAATGTGGTGGTTGTTGTGATGATTATACTGGATACCAGGAGTTAGTAATCAA |
| | AACTTCACATGACGACTAAGTTCGTCTTTGATTTATTGCATTGATCTTTTGTTAGATCTTTTTCCAA |
| | TCTAGCATTCTTTACCACGCCCTTTTAATGGACATTTGGAGACCTGAGAAGAAATTTCTCCGTTACA |
| | TTAACGGTTTTAATGTCTCAGAATTAGAAGATGCTTGTTTTAAATTTAACTATCAATTTCCTAAAGT |
| | AGGATATTGTAGAGTTCCTAGTCATGCTTGGTGCCGTAATCAAGGTAGATTTTGTGCTACATTCACT |
| | CTTTATGGTAAATCCAAACATTATGATAAATATTTTGGAGTAATAAATGGTTTCACAGCATTCGCTA |
| | ATACTGTAGAGGATGCTGTTAACAAACTGGTTTTCTTAGCTGTTGACTTTATTACCTGGCGTAGACA |
| | GGAGTTAAATGTTTATGGCTGATGCTTATCTTGCAGACACTGTGTGGTATGTGGGGCAAATAATTTT |
| | TATAGTTGCCATTTGTTTATTGGTTACAATAGTTGTAGTGGCATTTTTGGCAACTTTTAAATTGTGT |
| | ATTCAACTTTGCGGTATGTGTAATACCTTAGTACTGTCCCCTTCTATTTATGTGTTTAATAGAGGTA |
| | GGCAGTTTTATGAGTTTTACAATGATATAAAACCACCAGTCCTTGATGTGGATGACGTTTAGGTAAT |
| | CCAAACATTATGAGTAGTAAACCTACACCAGCACCAGTTTATATCTGGACTGCTGATGAAGCTATTA |
| | AATTCCTAAAGGAATGGAATTTTTCTTTGGGTATTATACTACTTTTTATTACAATCATATTGCAATA |
| | TGGATATACAAGTCGCAGTATGTTTGTTTATGTTATTAAGATGATTATTTTGTGGCTTATGTGGCCC |
| | ATTACTATAATCTTAACTACTTTCAATTGCGTATACGCATTGAATAACGTGTATCTTGGCCTTTCTA |
| | TAGTTTTTACCATAGTGGCCATTATTATGTGGATTGTGTATTTTGTGAATAGTATCAGGTTGTTTAT |
| | TAGAACTGGAAGTTTTTGGAGTTTTAACCCAGAAACAAACAACTTGATGTGTATAGATATGAAAGGA |
| | ACAATGTATGTTAGGCCGATAATTGAGGACTATCATACCCTGACGGTCACAATAATACGCGGTCATC |
| | TTTATATTCAAGGTATAAAACTAGGTATTGGCTATTCTTTGGCAGATTTGCCAGCTTATATGTCTGT |
| | TGCTAAGGTTACACACCTGTGCACATATAAGCGTGGTTTTCTTGACAGGATAAGCGATACTAGTGGT |
| | TTTGCTGTTTATGTTAAGTTCAAAGTCGGTAATTACCGACTGCCATCAACCCAAAAGGGTTCTGGCA |
| | TGGACACCGCATTGTTGAGAAATAATATCTAAATTTTAAGGATGTCTTTTACTCCTGGTAAGCAATC |
| | CAGTAGTAGAGCGTCCTCTGGAAATCGGTCTGGTAATGGCATCCTCAAGTGGGCCGATCAGTCCGAC |
| | CAGTTTAGAAATTTTCAAACCAGGGGTAGAAGAGCTCAACCCAAGCAAACTGCTACTTCTCAGCAAC |
| | CATCAGGAGGGAATGTTGTACCCCACTATTCTTGGTTCTCTGGAATTACTCAGTTTCAAAAGGGAAA |
| | GGAGTTTGAGTTTGCAGAAGGACAAGGTGTGCCTATTGCACCAGGAGTCCCAGCTACTGAAGCTAAG |
| | GGGTACTGGTACAGACACAACAGACGTTCTTTTAAAACAGCCGATGGCAACCAGCGTCAACTGCTGC |
| | CACGATGGTATTTTTACTATCTGGGAACCGGACCGCATGCTAAAGACCAGTATGGCACCGATATTAA |
| | CGGAGTCTACTGGGTCGCTAGCAACCAGGCTGATGTCAATACCCCGGCTGACATTGTCGATCGGGAC |
| | CCAAGTAGCGATGAGGCTATTCCGACTAGGTTTCCGCCTGGCACGGTACTCCCTCAGGGTTACTATA |
| | TTGAAGGCTCAGGAAGGTCTGCTCCTAATTCCAGATCTACTTCGCGCACATCCAGCAGAGCCTCTAG |
| | TGCAGGATCGCGTAGTAGAGCCAATTCTGGCAATAGAACCCCTACCTCTGGTGTAACACCTGACATG |
| | GCTGATCAAATTGCTAGTCTTGTTCTGGCAAAACTTGGCAAGGATGCCACCAAACCTCAGCAAGTAA |
| | CTAAGCATACTGCCAAAGAAGTCAGACAGAAAATTTTGAATAAACCCCGCCAGAAGAGGAGCCCCAA |
| | TAAACAATGCACTGTTCAGCAGTGTTTTGGTAAGAGAGGCCCTAATCAGAATTTTGGTGGTGGAGAA |
| | ATGTTAAAACTTGGAACTAGTGACCCACAGTTCCCCATTCTTGCAGAACTCGCACCCACAGCTGGTG |
| | CGTTTTTCTTTGGATCAAAATTAGAGTTGGCCAAAGTGCAGAATTTATCTGGGAATCCTGATGAGCC |
| | TCAGAAGGATGTTTATGAATTGCGCTATAACGGCGCAATTAGGTTTGACAGTACACTTTCAGGCTTT |
| | GAGACCATAATGAAGGTGCTGAGTGAGAATTTGAATGCCTATCAACAACAAGATGGTATGATGAATA |
| | TGAGTCCAAAACCACAGCGTCAGCGTGGTCATAAGAATGGACAAGGAGAAAATGATAATATAAGTGT |
| | TGCAGTGCCCAAAAGCCGCGTGCAGCAAAATAAGAGTATAGAGTTGACTGCAGAGGACATCAGCCTT |
| | CTTAAGAAGATGGATGAGCCCTTTACTGAAGACACCTCAGAAATATAAGAGAATGAACCTTATGTCG |
| | GCATCTGGTGGTAACCCCTCGCAGAAAAGTCGAGATAAGGCACTCTCTATCAGAATGGATGTCTTGC |
| | TGCTATAATAGATAGAGAAGGTTATAGCAGACTATATATTAATTAGTTGAAAGTTTTGTGTTGTAAT |
| | GTATAGTGTTGGAGAAAGTGAAAGACTTGCGGAAGTAATTGCCGACAAGTGCCAAAGGGAAGAGCCA |
| | GCATGTTAAGTTACTACCCAGTAATTAGTAAATGAATGAAGTTAATTATGGCCAATTGGAAG |
| 8 | GenBank Accession No. MN306018.1 (Human coronavirus NL63 strain |
| | HCoV_NL63/Seattle/USA/SC0179/2018, complete genome) |
| | ACTTTGTGTCTACTCTTCTCAACTAAACGAAATTTTTCTAGTGCTGTCATTTGTTATGGCAGTCCTA |
| | GTGTAATTGAAATTTCGTTTAGTTTGTAAACTGGTTAGGCAAGTGTTGTATTTTCTGTGTCTAAGCA |
| | CTGGTGATTCTGTTCACTAGTGCATACACTGATATTTAAGTGGTGTTCCGTCACTGCTTATTGTGGA |
| | AGCAACGTTCTGTCGTTGTGGAAACCAATAACTGCTAACCATGTTTTACAATCAAGTGACACTTGCT |
| | GTTGCAAGTGATTCGGAAATTTCAGGTTTTGGTTTTGCCATTCCTTCTGTAGCCGTTCGCACTTATA |
| | GCGAAGCCGCTGCACAAGGTTTTCAGGCATGCCGTTTTGTTGCTTTTGGCTTACAGGATTGTGTAAC |
| | CGGTATTAATGATGATGATTATGTCATTGCATTGACTGGTACTAATCAACTCTGTGCCAAAATTTTA |
| | CCTTTTTCTGATAGACCCCTTAATTTGCGAGGTTGGCTCATTTTTTCTAACAGCAATTATGTTCTTC |
| | AGGACTTTGATGTTGTTTTTGGCCATGGTGCAGGAAGTGTGGTTTTTGTGGATAAGTATATGTGTGG |
| | TTTTGATGGTAAACCTGTGTTACCTAAAAACATGTGGGAATTTAGGGATTACTTTGATAATAATACT |
| | GATAGTATTGTTATTGATGGTGTCACTTATCAACTAGCATGGGATGTTATACGTAAAGACCTTTCTT |
| | ATGAACAGCAAAATGTTTTAGCCATTGAGAGCATTCATTACCTTGGTACTACAGGTCATACTTTGAA |
| | GTCCGGTTGCAAACTTATTAATGCCAAGCCGCCTAAATATTCTTCTAAGGTTGTTTTGAGTGGTGAA |
| | TGGAATGCTGTGTATAGGGCATTTGGTTCACCATTTATTACAAATGGTATGTCATTGCTAGATATAA |
| | TTGTTAAACCAGTTTTCTTTAATGCTTTTGTTAAATGCAATTGTGGTTCTGAGAGTTGGAGTGTTGG |
| | TGCATGGGATGGTTACTTATCTTCTTGTTGTGGCACACCTGCTAAGAAACTTTGTGTTGTTCCTGGT |
| | AATGTTGTTCCTGGTGATGTGATCATCACCTCAACTGGTGCTGGTTGTGGTGTTAAATACTATGCTG |
| | GCTTAGTTGTTAAACATATTACTAACATTACTGGTGTGTCTTTATGGCGTGTTACAGCTGTTCATTC |
| | TGATGGAATGTTTGTGGCATCATCTTCTTATGATGCACTCTTGCATAGAAATTCATTAGACCCTTTT |
| | TGCTTTGATGTTAACACTTTACTTTCTAATCAATTACGTCTAGCTTTTCTTGGTGCTTCTGTTACAG |
| | AAGATGTTAAATTTGCTGCTAGCACTGGTGTTATTGACATTAGTGCTGGTATGTTTGGTCTTTATGA |
| | TGACATATTGACAAACAACAAACCTTGGTTTGTACGCAAAGCTTCTGGGCTTTTTGATGCAATCTGG |
| | GATGCTTTTGTTGCCGCTATTAAGCTTGTACCAACTACTACTGGTGTTTTGGTTAGGTTTGTTAAGT |
| | CTATTGCTTCAACTGTTTTAACTGTCTCTAATGGTGTTATTATTATGTGTGCAGATGTTCCAGATGC |
| | TTTTCAATCAGTTTATCGCACATTTACACAAGCTATTTGTGCTGCATTTGATTTTTCTTTAGATGTA |
| | TTTAAAATTGGTGATGTTAAATTTAAACGACTTGGTGATTATGTTCTTACTGAAAACGCTCTTGTTC |
| | GTTTGACTACTGAAGTTGTTCGTGGTGTTCGTGATGCTCGCATAAAGAAAGCCATGTTTACTAAAGT |
| | AGTTGTAGGTCCTACAACTGAAGTTAAGTTTTCTGTTATTGAACTTGCCACTGTTAATTTGCGTCTT |
| | GTTGATTGTGCACCTGTAGTTTGCCCTAAAGGTAAGATTGTTGTTATTGCTGGACAAGCTTTTTTCT |
| | ATAGTGGTGGTTTTTATCGTTTTATGGTTGATCCTACAACTGTATTAAATGATCCTGTTTTTACTGG |
| | TGATTTATTTTACACTATTAAGTTTAGTGGTTTTAAGCTTGATGGTTTTAACCATCAGTTTGTTACT |
| | GCTAGTTCTGCTACTGATGCTATTATTGCTGTTGAGCTGTTGTTATTGGATTTTAAAACTGCAGTTT |
| | TTGTGTACACATGTGTGGTTGATGGCTGTAGTGTCATTGTTAGACGTGATGCTACATTCGCTACACA |
| | TGTGTGTTTTAAGGACTGTTATAATATTTGGGAGCAATTCTGCATTGATAATTGTGGTGAGCCATGG |
| | TTTTTGACTGATTATAATGCTATCTTGCAGAGTAATAACCCTCAATGTGCTATTGTTCAAGCATTGG |
| | AGTCTAAAGTTTTGCTTGAGAGGTTTTTACCTAAGTGTCCTGAAATACTGTTGAGTATTGATGATGG |
| | CCATTTATGGAATCTTTTTGTTGAAAAGTTTAATTTTGTTACAGATTGGTTAAAAACTCTTAAGCTT |
| | ACACTTACTTCTAATGGTCTTTTAGGTAATTGTGCCAAACGTTTTAGACGTGTTTTGGTAAAATTGC |
| | TTGATGTCTATAATGGTTTTCTTGAAACTGTCTGTAGTGTCGCATACACTGCTGGTGTTTGCATCAA |
| | ATATTATGCTGTTAATGTTCCATATGTAGTTATTAGTGGTTTTGTAAGTCGTGTAATTCGTAGAGAA |
| | AGGTGTGACATGACTTTTCCTTGTGTTAGTTGTGTCACCTTTTTCTATGAATTTTTAGACACTTGTT |
| | TTGGTGTTAGTAAACCTAATGCCATTGATGTTGAACATTTAGAGCTTAAAGAAACCGTTTTTGTTGA |
| | ACCTAAGGATGGTGGTCAATTTTTTGTTTCTGGTGATTATCTTTGGTATGTTGTAGATGACATTTAT |
| | TATCCAGCTTCATGTAATGGTGTATTGCCAGTTGCTTTTACAAAATTAGCAGGTGGTAAAATATCTT |
| | TTTCTGATGATGTTATAGTTCATGATGTTGAACCTACCCATAAAGTCAAGCTCATATTTGAGTTTGA |
| | AGATGATGTTGTTACCAGTCTTTGTAAGAAGAGTTTTGGTAAGTCCATTATTTATACAGGTGATTGG |
| | GAAGGTCTACATGAAGTTCTTACATCTGCAATGAATGTCATTGGGCAACATATTGAGTTGCCACAAT |
| | TTTATATTTATGATGAAGAGGGTGGTTATGATGTTTCTAAACCAGTTATGATTTCACAATGGCCTAT |
| | TAGTAATGATAGTAATGGTTGTGTTGTTGAAGCGAGCACTGATTTTCATCAATTAGAATGTATTGTT |
| | GATGATTCTGTTAGAGAAGAGGTTGATATAATTGAACAACCTTTTGAAGAAGTTGAACATGTGCTCT |
| | CAATTAGACAACCTTTTTCTTTTTCTTTTAGAGATGAATTGGGTGTTCGTGTTTTAGATCAATCTGA |
| | TAATAATTGTTGGATTAGTACCACACTTGTACAGTTGCAACTTACAAAGCTTTTGGATGATTCTATT |
| | GAGATGCAATTGTTTAAAGTTGGTAAAGTTGATTCAATTGTCCAAAAGTGTTATGAGTTGTCTCATT |
| | TAATTAGTGGTTCACTTGGTGATAGTGGTAAACTTCTTAGTGAACTTCTTAAAGAAAAATATACATG |
| | TTCTATAACTTTTGAGATGTCTTGTGATTGTGGTAAAAAGTTTGATGAGCAGGTTGGTTGTTTGTTT |
| | TGGATTATGCCTTACACAAAACTTTTTCAAAAAGGTGAGTGTTATATTTGTCATAAAATGCAGACTT |
| | ATAAGCTTGTTAGTATGAAAGGTACTGGTGTGTTTGTACAGGACCCAGCACCTATTGACATTGATGC |
| | TTTCCCTGTGAAACCTATATGTTCATCTGTATATTTAGGTGTTAAGGGTTCTGGTCATTATCAAACA |
| | AATTTATACAGTTTTAATAAAGCTATTGATGGTTTTGGTGTCTTTGACATTAAAAATAGTAGTGTTA |
| | ATACTGTTTGTTTTGTCGATGTTGATTTTCATAGTGTAGAAGTAGAAGCTGGTGAAGTTAAACCTTT |
| | TGCTGTATATAAAAATGTTAAATTTTATTTAGGTGACATTTCACACCTTATAAACTGTGTTTCTTTT |
| | GACTTTGTTGTCAATGCTGCTAATGAGAATCTTATGCATGGAGGCGGTGTTGCACGTGCTATTGATA |
| | TTTTGACTGAAGGTCAACTTCAGTCATTATCTAAAGATTACATTAGTAGTAATGGTCCACTTAAGGT |
| | AGGAACAGGTGTTATGTTGGAGTGTGAAAAATTCAATGTATTTAATGTTGTTGGTCCGCGAGCTGGT |
| | AAACATGAGCATTCATTACTTGTTGAAGCTTATAATTCTATCTTAGTTGAAAATGGTATTCCACTTA |
| | TGCCTCTTCTTAGTTGTGGTATTTTTGGTGTAAGGATTGAAAATTCTCTTAAAGCTTTGTTTAGTTG |
| | TGACNTTAATAAATTATTGCAAGTTTTTGTTTATTCTTCAAATGAAGAACAAGCTGTTCTTAAGTTT |
| | TTAGATGGTTTAGATTTAACACCAGTCATTGATGATGTTGATGTTGTTAAACCTTTTAGAGTTGAAG |
| | GTAATTTTTCATTCTTTGATTGTGGTGTCAATGCCTTGGATGGTGATATTTACTTATTATTCACTAA |
| | CTCTATTTTAATGTTGGATAAACAAGGACAATTATTGGATACAAAACTTAATGGTATTTTGCAACAG |
| | GCAGCTCTTGATTATCTTGCTACAGTTAAAACTGTACCAGCTGGTAATTTGGTTAAACTTGTTGTTG |
| | AGAGTTGTACCATTTATATGTGTGTTGTACCATCGATAAATGATCTTTCTTTTGATAAAAATCTTGG |
| | TCGTTGTGTGCGTAAACTTAATAGATTGAAAACTTGTGTTATTGCCAATGTTCCTGCTATTGATGTT |
| | TTGAAAAAGCTTCTTTCAAGTTTGACTTTAACTGTTAAATTTGTTGTAGAGAGTAATGTTATGGATG |
| | TTAACGACTGTTTTAAGAATGATAATGTAGTTTTGAAAATTACTGAAGATGGTATTAATGTTAAAGA |
| | TGTTGTTGTTGAGTCTTCTAAGTCACTTGGTAAACAATTGGGTGTTGTGAGTGATGGTGTTGACTCT |
| | TTTGAAGGTGTTTTGCCTATTAATACTGATACTGTCCTATCTGTAGCTCCAGAAGTTGACTGGGTTG |
| | CTTTTTATGGTTTTGAAAAGGCAGCACTTTTTGCTTCTTTGGATGTAAAGCCATATGGTTACCCTAA |
| | TGATTTTGTTGGTGGTTTTAGAGTTCTTGGGACCACCGACAATAATTGTTGGGTTAATGCAACTTGT |
| | ATAATTTTACAGTATCTTAAGCCTACTTTTAAATCTGAGGGTTTAAATGTTCTTTGGAACAAATTTG |
| | TTACAGGTGATGTTNGACCTTTTGTTAGTTTTATTTATTTTATAACTATGTCTTCAAAGGGTCAAAA |
| | GGGTGATGCTGAAGAGGCATTATCTAAATTGTCAGAGTATTTGATTAGTGATTCTATTGTTACTCTT |
| | GAACAATATTCAACTTGTGACATTTGTAAAAGTACTGTAGTTGAAGTTAAAAGTGCTGTTGTTTGTG |
| | CTAGTGTGCTTAAAGATGGTTGTGATGTTGGTTTTTGTCCACACAGACATAAATTGCGTTCACGTGT |
| | TAAGTTTGTTAATGGACGTGTTGTTATTACCAATGTTGGTGAACCTATAATTTCACAACCTTCTAAG |
| | TTGCTTAATGGTATTGCTTATACAACATTTTCAGGTTCTTTTGATAACGGTCACTATGTAGTTTATG |
| | ATGCTGCTAATAATGCTGTCTATGATGGTGCTCGTTTGTTTGCTTCAGATTTGTCTACTTTAGCTGT |
| | TACAGCTATTGTTGTAGTAGGTGGTTGTGTAACATCTAATGTTTCACCAATTGTTAGTGAGAAAATT |
| | TCTGTTATGGATAAACTTGATACTGGTGCACAAAAATTTTTCCAGTTTGGTGATTTTGTTATGAATA |
| | ACATTGTTCTGTTTTTAACTTGGTTGCTTAGTATGTTTAGTCTTTTACGTACTTCTATTATGAAGCG |
| | TGATATTAAAGTTATTGCCAAGGCTCCTAAACGTACAGGTGTTATTTTGACACGTAGTTTTAAGTAT |
| | AACATTAAATCTGCTTTGTTTGTTGTAAAACAGAAGTGGTGTGTTATTGTTACTTTGTTTAAGTTCT |
| | TATTGTTATTATATGCTATTTATGCACTTGTTTTTATGATTGTGCAATTTAGTCCTTTTAATAGTCT |
| | TTTATGTGGTGACATTGTAAGTGGTTATGAAAAATCCACTTTTAATAAGGATATTTATTGTGGTAAT |
| | TCTATGGTTTGTAAGATGTGTTTGTTTAGGTATCAAGAGTTTAATGATTTGGATCATACTAGTCTTG |
| | TTTGGAAGCACATTCGTGATCCTATATTAATCAGTTTACAACCATTTGTTATACTTGTTATTTTGTT |
| | AATTTTTGGTAATATGTATTTGCGTTTTGGACTTTTATATTTTGTTGCACAATTTATTAGTACTTTT |
| | GGTTCTTTCTTAGGCTTTCATCAGAAACAGTGGTTTTTACATTTTGTGCCGTTCGATGTTTTATGTA |
| | ATGAGTTTTTAGCTACATTTATTGTCTGCAAAATTGTTTTATTTGTTAGACATATTATTGTTGGCTG |
| | TAATAATGCTGACTGTGTAGCTTGTTCTAAAAGTGCTAGACTTAAACGTGTACCACTTCAAACTATT |
| | ATTAATGGTATGCATAAATCATTCTATGTTAATGCTAATGGTGGTACTTGTTTCTGTAGTAAACATA |
| | ACTTCTTTTGTGTTAATTGTGATTCTTTTGGGCCTGGTAATACTTTTATTAATGGTGATATTGCAAG |
| | AGAGCTTGGTAATGTTGTTAAAACAGCTGTTCAACCCACAGCTCCTGCATATGTTATTATTGATAAG |
| | GTAGATTTTGTTAATGGATTTTATCGTCTTTATAGTGGTGACACTTTTTGGCGTTATGACTTTGACA |
| | TTACTGAATCTAAGTATAGTTGTAAAGAGGTTCTGAAGAATTGTAATGTTTTAGAAAATTTTATTGT |
| | TTACAATAATAGTGGTAGTAACGTTACACAGATTAAAAATGCTTGTGTTTATTTTTCTCAATTGTTG |
| | TGTGAACCNNNNNNNNNNNNNNNNNNNNNNNNGTTGTCAACTTTATCAGTTGATTTTAATGGTGTTT |
| | TGCATAAGGCATATGTTGATGTTTTGTGTAATAGTTTTTTTAAAGAGCTAACTGCTAACATGTCCAT |
| | GGCTGAATGTAAAGCTACACTTGGTTTGACTGTTTCTGATGATGATTTTGTTTCAGCTGTCGCCAAT |
| | GCACATAGGTATGACGTTTTGCTTTCAGATTTGTCATTTAATAATTTTTTTATTTCTTATGCTAAAC |
| | CTGAAGATAAGTTGTCCGTTTATGACATTGCTTGTTGTATGCGTGCCAGTTCTAAGGTTGTTAACCA |
| | TAATGTTTTAATCAAAGAGTCAATACCTATTGTTTGGGGTGTCAAGGACTTTAATACTCTTTCTCAA |
| | GAGGGTAAGAAGTACCTTGTTAAAACAACTAAAGCAAAGGGTTTGACTTTTCTATTAACTTTTAATG |
| | ACAACCAAGCAATTACACAAGTTCCTGCTACTAGTATAGTTGCAAAACAGGGTGCTGGTTTTAAACG |
| | TACTTATAATTTTCTATGGTATGTATGTTTATTTGTTGTTGCATTGTTTATTGGNNNCTCATTTATT |
| | GATTATACAACCACTGTAACTAGCTTTCATGGTTATGATTTTAAGTACATTGAGAATGGTCAGTTGA |
| | AGGTGTTTGAAGCACCTTTACACTGTGTTCGTAATGTTTTTGATAATTTTAATCAATGGCATGAGGC |
| | TAAGTTTGGTGTTGTTACTACTAATAGTGATAAATGTCCTATAGTTGTTGGTGTTTCAGAGCGTATT |
| | AATGTTGTTCCTGGTGTTCCAACAAATGTATATTTGGTAGGAAAGACTCTTGTTTTTACATTACAGG |
| | CTGCTTTTGGAAACACAGGTGTTTGTTATGACTTTGATGGTGTTACCACTAGTGATAAGTGTATTTT |
| | TAATTCTGCTTGTACTAGGTTGGAAGGTTTGGGTGGTGACAATGTTTATTGTTACAACACTGATCTT |
| | ATTGAAGGTTCTAAACCTTATAGTACTTTACAGCCTAATGCTTATTATAAGTATGATGTTAAAAATT |
| | ATGTACGTTTTCCAGAAATTTTAGCTAGAGGTTTTGGTTTACGTACTATTAGAACTTTGGCTACACG |
| | TTATTGTAGAGTTGGTGAATGCCGTGACTCACGTAAAGGTGTTTGTTTTGGTTTTGATAAATGGTAT |
| | GTTAATGATGGACGTGTTGATGACGGTTACATTTGTGGTGATGGTCTTATAGACCTTCTTGTTAATG |
| | TACTCTCAATCTTTAGTTCATCTTTTAGCGTTGTGGCTATGTCTGGACATATGTTGTTTAATTTTCT |
| | TTTTGCAGCATTTATTACATTTTTGTGCTTTTTAGTTACTAAATTTAAACGTGTTTTTGGTGATCTT |
| | TCTTATGGTGTTTTTACTGTTGTTTGTGCAACTTTGATTAATAACATTTCTTATGTTGTTACTCAAA |
| | ATTTATTTTTTATGTTGCTTTATGCTATTTTGTATTTTGTTTTTACTAGGACAGTGCGTTATGCTTG |
| | GATTTGGCATATTGCATATATTGTTGCATACTTCTTGTTAATACCATGGTGGCTTCTCACATGGTTT |
| | AGTTTTGCTGCATTTTTAGAGCTTTTACCTAATGTTTTTAAGTTAAAAATCTCTACTCAATTGTTTG |
| | AAGGTGATAAGTTTATAGGTACTTTTGAGAGTGCTGCTGCAGGTACATTTGTTCTTGACATGCGTTC |
| | TTATGAAAGGCTGATAAATACTATTTCACCTGAGAAACTTAAGAATTATGCTGCAAGTTATAATAAA |
| | TATAAATATTATAGTGGTAGTGCTAGTGAGGCTGACTATCGTTGTGCTTGTTATGCTCATTTAGCCA |
| | AGGCTATGTTAGATTACGCAAAAGATCATAATGACATGTTATATTCTCCACCTACCATTAGCTACAA |
| | TTCCACCTTACAATCTGGTCTTAAGAAGATGGCACAACCATCTGGTTGTGTTGAGAGATGTGTGGTT |
| | CGCGTCTGTTATGGTAGTACTGTGCTTAATGGAGTTTGGTTAGGTGACACTGTTACTTGTCCTAGAC |
| | ATGTCATAGCACCATCAACCACTGTTCTTATTGATTATGATCATGCATATAGTACTATGCGTTTGCA |
| | TAATTTTTCAGTGTCTTATAATGGTGTCTTCTTGGGAGTTGTTGGTGTTACAATGCATGGCTCTGTG |
| | TTGCGTATTAAGGTTTCACAATCTAATGTACATACACCTAAACATGTTTTTAAAACGTTGAAACCTG |
| | GTGATTCTTTTAATATTTTAGCATGTTATGAAGGTATTGCATCTGGTGTTTTTGGTGTTAATTTACG |
| | TACAAACTTTACTATTAAAGGTTCTTTTATAAATGGAGCTTGTGGTTCTCCTGGTTATAATGTTAGA |
| | AATGATGGTACTGTTGAGTTTTGTTATTTACACCAAATTGAGTTAGGTAGTGGTGCTCATGTTGGTT |
| | CTGATTTTACTGGTAGTGTTTATGGTAATTTTGATGACCAACCTAGTTTGCAAGTTGAGAGTGCCAA |
| | CCTTATGCTATCAGATAATGTTGTTGCCTTTTTGTACGCTGCTTTGTTGAATGGTTGTAGGTGGTGG |
| | TTGCGTTCAACTAGAGTTAATGTTGATGGTTTTAATGAATGGGCTATGGCTAATGGTTATACAAGTG |
| | TTTCTAGTGTTGAGTGCTATTCTATTTTGGCAGCAAAAACTGGTGTTAGTGTTGAACAATTGTTAGC |
| | TTCCATTCAACATCTTCATGAAGGTTTTGGTGGTAAAAACATACTTGGTTATTCTAGTTTATGTGAT |
| | GAGTTCACGCTAGCTGAAGTTGTGAAGCAGATGTATGGTGTTAACTTGCAAAGTGGTAAGGTTATTT |
| | TTGGTTTAAAAACAATGTTTTTATTTAGCGTTTTCTTCACAATGTTTTGGGCAGAACTCTTTATTTA |
| | TACAAACACTATATGGATAAACCCTGTGATACTTACACCTGTATTTTGTCTACTTTTGTTTTTGTCA |
| | TTATTTTTAACTATGTTTCTTAAACATAAGTTTTTGTTTTTGCAAGTATTTTTATTACCTACTGTTA |
| | TTGCAACTGCTTTATATAATTGTGTTTTGGATTATTACATAGTAAAATTTTTGGCTGACCATTTTAA |
| | CTATAATGTTTCAGTATTACAAATGGATGTTCAGGGTTTAGTTAATGTTTTGGTCTGTTTATTTGTT |
| | GTATTTTTACACACATGGCGCTTTTCTAAAGAACGTTTTACACATTGGTTTACATATGTGTGTTCTC |
| | TTATAGCAGTTGCTTACACTTATTTTTATAGTGGTGACTTTTTGAGTTTGCTTGTTATGTTTTTATG |
| | TGCTATATCTAGTGATTGGTACATTGGTGCCATTGTTTTTAGGTTATCACGTTTGATTGTAATTTTT |
| | TCACCTGAAAGTGTATTTAGTGTTTTTGGTGATGTGAAACTTACTTTAGTTGTTTATTTAATTTGTG |
| | GTTATTTAGTTTGTACTTATTGGGGCATTTTGTATTGGTTCAATAGGTTTTTTAAATGTACTATGGG |
| | TGTTTATGATTTTAAGGTGAGTGCTGCTGAATTTAAATACATGGTTGCTAATGGACTTCATGCACCA |
| | CATGGACCTTTTGATGCACTTTGGTTATCATTCAAACTACTTGGTATTGGTGGTGACCGTTGTATAA |
| | AAATTTCAACTGTCCAATCCAAACTGACTGATTTGAAGTGTACTAATGTTGTGTTATTGGGTTGTTT |
| | GTCTAGTATGAACATTGCAGCTAATTCTAGTGAATGGGCTTATTGTGTTGATTTACACAATAAGATT |
| | AATCTTTGTGATGACCCTGAAAAAGCTCAAGGTATGTTGTTAGCACTCCTTGCGTTCTTTCTAAGTA |
| | AACATAGTGATTTTGGTCTTGATGGCCTTATTGATTCTTATTTTGATAATAGTAGCACCCTTCAGAG |
| | TGTTGCTTCATCATTTGTTAGTATGCCATCATATATTGCTTATGAAAATGCTAGACAAGCTTATGAG |
| | GATGCTATTGCTAATGGATCTTCCTCTCAACTTATTAAACAATTGAAGCGTGCCATGAATATTGCAA |
| | AGTCTGAATTTGATCATGAGGTATCTGTTCAGAAGAAAATTAATAGAATGGCTGAACAAGCTGCTAC |
| | TCAGATGTATAAAGAAGCACGCTCTGTTAATAGAAAATCTAAAGTTATTAGTGCTATGCACTCTTTA |
| | CTTTTTGGAATGTTAAGACGTTTGGATATGTCTAGTGTTGAAACTGTTTTGAATTTAGCACGTGATG |
| | GTGTTGTGCCATTGTCAGTTATACCTGCAACTTCAGCTTCTAAACTAACTATTGTTAGTCCAGATCT |
| | TGAATCTTATTCTAAGATTGTTTGTGATGGTTCTGTTCATTATGCTGGAGTTGTTTGGACACTTAAT |
| | GATGTTAAAGACAATGATGGTAGACCTGTTCATGTTAAAGAGATTACAAAGGAAAATGTTGAAACTT |
| | TGACATGGCCTCTTATCCTTAATTGTGAACGTGTTGTTAAACTTCAAAATAATGAAATTATGCCTGG |
| | TAAACTTAAGCAAAAACCTATGAAAGCTGAGGGTGATGGTGGTGTTTTAGGTGATGGTAATGCCTTG |
| | TATAATACTGAGGGTGGTAAAACTTTTATGTATGCTTATATTTCTAATAAAGCTGACCTTAAATTTG |
| | TTAAGTGGGAGTATGAGGGTGGTTGCAACACAATCGAGTTAGACTCTCCTTGTCGATTTATGGTCGA |
| | AACACCTAATGGTCCTCAAGTGAAGTATTTGTATTTTGTTAAAAATTTAAATACCTTACGTAGAGGT |
| | GCCGTTCTTGGTTTTATAGGTGCCACAATTCGTCTACAAGCTGGTAAACAAACTGAATTGGCTGTTA |
| | ATTCTGGACTTTTAACTGCTTGTGCTTTTTCGGTTGATCCAGCAACTACTTACTTGGAGGCTGTTAA |
| | ACATGGTGCAAAACCTGTAAGTAATTGTATTAAGATGTTATCTAATGGTGCTGGTAATGGTCAAGCT |
| | ATAACAACTAGTGTAGATGCTAACACCAATCAAGATTCTTATGGTGGAGCATCTATTTGTTTGTATT |
| | GTCGGGCCCACGTTCCTCACCCTAGTATGGATGGTTACTGTAAGTTTAAGGGTAAATGTGTTCAGGT |
| | TCCTATTGGTTGTTTGGATCCTATTAGGTTTTGTTTAGAAAATAATGTGTGTAATGTTTGTGGTTGT |
| | TGGTTGGGACACGGGTGTGCTTGTGACCGTACAACTATTCAAAGTGTTGACATTTCTTATTTAAACG |
| | AGCAAGGGGTTCTAGTGCAGCTCGACTAGAACCCTGTAATGGCACGGACATCGATAAGTGTGTTCGT |
| | GTTTTTGACATTTATAATAAAAATGTTTCATTCTTGGGTAAGTGTTTGAAGATGAACTGTGTTCGTT |
| | TTAAAAATGCTGATCTTAAGGATGGTTATTTTGTTATAAAGAGGTGTACTAAGTCGGTTATGGAACA |
| | CGAGCAATCCATGTATAACCTACTTAACTTTTCTGGTGCTTTGGCTGAGCATGATTTCTTTACTTGG |
| | AAAGATGGCAGAGTCATTTATGGTAATGTTAGTAGACATAATCTTACTAAATATACTATGATGGACT |
| | TGGTCTATGCTATGCGTAACTTTGATGAACAAAATTGTGATGTTCTAAAAGAAGTATTAGTTTTAAC |
| | TGGTTGTTGTGACAATTCTTATTTTGATAGTAAGGGTTGGTATGACCCAGTTGAAAATGAGGATATA |
| | CATAGAGTTTATGCATCTCTTGGCAAAATTGTAGCTAGAGCTATGCTTAAATGCGTTGCTCTATGTG |
| | ATGCGATGGTTGCTAAAGGTGTTGTTGGTGTTTTAACATTAGATAACCAAGATCTTAATGGTAACTT |
| | TTATGATTTTGGTGATTTTGTTGTTAGCTTACCTAATATGGGTGTTCCCTGTTGTACATCATATTAT |
| | TCTTATATGATGCCTATTATGGGTTTAACTAATTGTTTAGCTAGTGAGTGTTTTGTCAAGAGTGATA |
| | TTTTTGGTAGTGATTTTAAAACTTTTGATTTGCTTAAGTATGATTTCACTGAACATAAAGAAAATTT |
| | ATTCAATAAGTACTTTAAGCATTGGAGTTTTGATTATCATCCTAATTGTAGTGACTGTTATGATGAT |
| | ATGTGTGTTATACATTGTGCTAATTTTAATACACTATTTGCTACAACTATACCAGGTACTGCTTTTG |
| | GTCCACTATGTCGTAAAGTTTTTATAGATGGTGTTCCACTTGTTACAACTGCTGGTTATCATTTTAA |
| | GCAATTAGGTTTGGTTTGGAATAAAGATGTTAACACACACTCAGTTAGGTTGACAATTACTGAACTT |
| | TTGCAATTTGTCACCGACCCTTCCTTGATAATAGCTTCTTCCCCAGCACTCGTTGATCAACGCACTA |
| | TTTGTTTTTCTGTTGCAGCATTGAGTACTGGTTTGACAAATCAAGTTGTTAAGCCAGGTCATTTTAA |
| | TGAAGAGTTTTATAACTTTCTTCGTTTAAGAGGTTTCTTTGATGAAGGTTCTGAACTTACATTAAAA |
| | CATTTCTTCTTCGCACAGAATGGTGATGCTGCTGTTAAAGATTTTGACTTTTACCGTTATAATAAGC |
| | CTACCATTTTAGAGATTTGTCAAGCTAGAGTTACATATAAGATAGTCTCTCGTTATTTTGACATTTA |
| | TGNAGGTGGCTGTATTAAGGCATGTGAAGTTGTTGTAACAAATCTTAATAAGAGTGCTGGTTGGCCA |
| | TTAAATAAGTTTGGTAAAGCTAGTTTGTATTATGAATCTATATCTTATGAAGAACAGGATGCTTTGT |
| | TTGCTTTGACAAAGCGTAATGTCCTCCCTACTATGACACAGCTGAATCTTAAGTATGCTATTAGTGG |
| | TAAAGAACGTGCTAGAACTGTTGGTGGTGTTTCTCTGTTGTCTACAATGACCACAAGACAATACCAT |
| | CAAAAACATCTTAAATCCATTGTTAATACACGCAATGCCACTGTTGTTATTGGTACTACCAAATTTT |
| | ATGGTGGTTGGAATAATATGTTGCGTACTTTAATTGATGGTGTTGAAAACCCTATGCTTATGGGTTG |
| | GGATTATCCCAAATGTGATAGAGCTTTGCCTAACATGATACGTATGATTTCAGCCATGGTGTTGGGC |
| | TCTAAACATGTTAATTGTTGTACTGCAACAGATAGGTTTTATAGGCTTGGTAATGAGTTGGCACAAG |
| | TTTTAACAGAAGTTGTTTATTCTAATGGTGGTTTTTATTTTAAGCCAGGTGGTACGACTTCTGGTGA |
| | CGCTAGTACAGCTTATGCTAATTCTATTTTTAACATTTTTCAAGCCGTGAGTTCTAACATTAACAGG |
| | TTGCTTAGTGTCCCATCAGATTCATGTAATAATGTTAATGTTAGGGATCTACAACGACGTCTGTATG |
| | ATAATTGCTATAGGTTAACTAGTGTTGAAGAGTCATTCATTGATGATTATTATGGTTATCTTAGGAA |
| | ACATTTTTCAATGATGATTCTCTCTGATGACGGTGTTGTCTGTTATAACAAGGATTATGCTGAGTTA |
| | GGTTATATAGCAGACATTAGTGCTTTTAAAGCCACTTTGTATTACCAGAATAATGTCTTTATGAGTA |
| | CTTCTAAATGTTGGGTTGAAGAAGATTTAACTAAGGGACCACATGAGTTTTGTTCCCAGCATACTAT |
| | GCAAATAGTTGACAAAGATGGTACCTATTATTTGCCTTACCCAGATCCTAGTAGGATCTTGTCAGCT |
| | GGTGTTTTTGTTGATGATGTTGTTAAGACAGATGCTGTTGTTTTGTTAGAACGTTATGTGTCTTTAG |
| | CTATTGATGCATACCCTCTTTCAAAACACCCTAATTCTGAATATCGCAAGGTTTTTTACGTATTACT |
| | TGATTGGGTTAAGCATCTTAACAAAAATTTGAATGAGGGTGTTCTTGAATCTTTTTCTGTTACACTT |
| | CTTGATAATCAAGAAGATAAGTTTTGGTGTGAAGATTTTTATGCTAGTATGTATGAAAATTCTACAA |
| | TATTGCAAGCTGCTGGTTTGTGTGTTGTTTGTGGTTCACAAACTGTACTTCGTTGTGGTGATTGTCT |
| | GCGTAAGCCTATGTTGTGCACTAAATGCGCATATGATCATGTATTTGGTACCGACCACAAGTTTATT |
| | TTGGCTATAACACCGTATGTATGTAATGCATCAGGTTGTGGTGTTAGTGATGTCAAAAAATTGTATC |
| | TTGGTGGTTTGAATTACTATTGTACAAATCATAAACCACAGTTGTCTTTTCCATTATGTTCAGCTGG |
| | TAATATATTTGGTCTATATAAAAATTCAGCAACTGGTTCCTTAGATGTTGAAGTTTTTAATAGGCTT |
| | GCAACGTCTGATTGGACTGATGTTAGGGACTATAAACTTGCTAATGATGTTAAAGATACACTTAGAC |
| | TCTTTGCGGCTGAAACTATTAAAGCTAAAGAAGAGAGTGTTAAGTCTTCTTATGCTTTTGCAACTCT |
| | TAAAGAGGTTGTTGGACCTAAAGAATTGCTTCTTAGTTGGGAAAGTGGTAAAGTTAAACCACCTTTG |
| | AATCGTAATTCTGTTTTCACCTGTTTTCAAATAAGTAAGGACTCAAAATTCCAAATAGGTGAGTTCA |
| | TCTTTGAGAAGGTTGAATATGGTTCTGATACTGTTACGTATAAGTCTACTGTAACTACTAAGTTAGT |
| | TCCTGGTATGATTTTTGTCTTAACATCTCACAATGTCCAACCTTTACGCGCACCAACTATTGCAAAC |
| | CAAGAGAAGTATTCTAGCATTTATAAATTGCACCCTGCTTTTAATGTCAGTGATGCATATGCTAATT |
| | TGGTTCCATATTACCAACTTATTGGTAAACAAAAGATAACTACAATACAGGGTCCTCCTGGTAGTGG |
| | TAAGTCACATTGTTCCATTGGACTTGGATTGTACTACCCAGGTGCGCGTATTGTTTTTGTTGCTTGT |
| | GCCCATGCTGCTGTTGATTCCTTATGTGCAAAAGCTATGACTGTTTATAGCATTGATAAGTGTACTA |
| | GGATTATACCTGCAAGAGCTCGGGTTGAGTGTTATAGTGGCTTTAAACCAAATAACACTAGTGCACA |
| | ATACATATTTAGCACTGTTAACGCATTACCTGAGTGTAATGCTGATATCGTTGTTGTAGATGAAGTT |
| | TCAATGTGTACAAATTATGAGCTTTCTGTTATTAATCAGCGTTTATCATATAAACATATTGTTTATG |
| | TTGGTGACCCACAACAACTTCCTGCACCTAGAGTAATGATTACTAAAGGTGTTATGGAGCCTGTTGA |
| | TTATAACGTTGTTACTCAACGTATGTGTGCTATAGGCCCTGATGTTTTTCTTCATAAATGTTATAGA |
| | TGTCCTGCTGAAATAGTTAATACAGTTTCTGAACTTGTTTATGAGAACAAGTTTGTCCCTGTTAAAC |
| | CTGCTAGTAAACAGTGTTTTAAAGTCTTTTTTAAGGGTAATGTACAGGTTGACAATGGTTCTAGTAT |
| | TAACAGAAAGCAGCTTGAAATAGTTAAGCTGTTTTTAGTTAAAAATCCAAGTTGGAGTAAGGCTGTG |
| | TTTATTNNNNNTTATAATAGTCAGAATTATGTTGCTAGTAGATTTTTAGGACTTCAAATTCAAACTG |
| | TTGATTCTTCTCAAGGTAGTGAGTATGATTATGTAATCTATGCACAAACTTCTGACACTGCACATGC |
| | TTGCAATGTAAACCGTTTTAATGTTGCTATAACACGTGCTAAGAAGGGTATATTTTGTGTAATGTGT |
| | GATAAAACTTTGTTTGATTCACTTAAGTTTTTTGAGATTAAACATGCAGATTTACATTCTAGCCAGG |
| | TTTGTGGCTTGTTTAAAAATTGTACACGCACTCCTCTTAATTTACCACCAACTCATGCACACACTTT |
| | CTTGTCGTTGTCAGATCAGTTTAAGACTACAGGTGATTTAGCTGTTCAAATAGGTTCAAATAACGTT |
| | TGTACTTATGAACATGTTATATCATTTATGGGTTTTAGGTTTGATATTAGTATTCCTGGTAGTCATA |
| | GTTTGTTTTGTACACGTGACTTTGCTATTCGTAATGTGCGTGGTTGGTTGGGTATGGATGTTGAAAG |
| | TGCTCATGTTTGTGGCGATAACATAGGTACTAATGTTCCTTTACAGGTTGGTTTTTCAAATGGTGTT |
| | AATTTTGTTGTGCAAACTGAAGGTTGTGTGTCTACCAATTTTGGTGATGTTATTAAACCTGTTTGTG |
| | CAAAATCTCCACCAGGTGAACAATTTAGACACCTTGTTCCTCTTTTACGTAAAGGACAACCTTGGTT |
| | AATTGTTCGTAGACGCATTGTGCAAATGATATCTGATTATTTGTCCAATTTGTCTGACATTCTTGTC |
| | TTTGTTTTGTGGGCAGGTAGTTTGGAATTAACTACAATGCGTTACTTTGTAAAAATAGGGCCAATTA |
| | AATATTGTTATTGTGGTAATTTTGCCACTTGTTATAATTCAGTTAGTAATGAATATTGTTGTTTTAA |
| | ACATGCATTGGGTTGTGATTATGTTTACAATCCGTATGCTTTTGATATACAACAGTGGGGTTATGTT |
| | GGTTCCTTGAGCCAAAACCACCACACATTCTGTAACATTCATAGAAACGAGCATGATGCTTCTGGTG |
| | ATGCTGTTATGACACGTTGTTTGGCAGTACATGATTGTTTTGTCAAAAATGTTGATTGGACTGTAAC |
| | GTACCCCTTTATTGCAAATGAGAAATTTATCAATGGCTGTGGGCGTAATGTCCAGGGACATGTTGTT |
| | CGTGCAGCCTTGAAATTGTATAAACCTAGTGTTATTCATGACATTGGTAATCCTAAAGGTGTACGTT |
| | GTGCTGTTACTGATGCCAAATGGTACTGTTATGACAAGCAACCTGTTAATAGTAATGTCAAGTTGTT |
| | GGATTATGATTATGCAACCCATGGTCAACTTGATGGTCTTTGTCTATTCTGGAATTGTAATGTTGAT |
| | ATGTATCCAGAATTTTCAATTGTGTGTCGTTTTGACACACGTACTCGTTCTGTTTTTAATTTAGAAG |
| | GTGTTAATGGTGGTTCTCTTTATGTTAACAAACATGCGTTTCATACACCAGCATATGATAAACGTGC |
| | TTTTGTTAAATTAAAACCTATGCCCTTTTTTTATTTTGATGACAGTGATTGTGATGTTGTGCAAGAA |
| | CAAGTTAATTATGTACCCCTTCGCGCTAGTAGTTGTGTTACCCGTTGTAATATAGGTGGTGCTGTTT |
| | GTTCAAAACATGCAAATTCGTATCAAAAATATGTTGAGGCATATAATACATTTACACAGGCAGGTTT |
| | TAACATTTGGGTACCACATAGTTTTGATGTTTATAATTTGTGGCAAATNNNNNNNNNNNNNNNNNNN |
| | NNNNNNNNNNNNNNNNNNGCATTTAATGTTGTAAAAAAAGGGTGTTTTACTGGTGTTGATGGTGAGT |
| | TACCTGTTGCAGTTGTTAACGACAAAGTTTTTGTTCGCTATGGCGATGTTGACAACTTGGTTTTCAC |
| | AAATAAAACAACATTGCCTACTAATGTTGCTTTTGAATTGTTTGCAAAACGAAAAATGGGTTTAACA |
| | CCACCATTGTCTATTCTCAAAAATCTCGGTGTTGTTGCTACATATAAATTTGTTTTATGGGATTATG |
| | AAGCTGAAAGACCTTTTACCTCATATACTAAGAGTGTATGTAAATACACTGATTTTAATGAGGATGT |
| | TTGTGTTTGTTTTGACAATAGTATTCAGGGTTCGTATGAGCGTTTTACGCTTACTACGAACGCTGTT |
| | TTATTTTCTACTGTTGTCATTAAAAATTTAACACCTATAAAGTTGAATTTTGGTATGTTGAATGGTA |
| | TGCCAGTTTCTTCTATTAAGGGTGATAAAGGTGTTGAAAAATTAGTTAATTGGTACATATATGTTCG |
| | TAAAAATGGTCAATTTCAAGATCATTATGATGGTTTTTACACTCAAGGTAGGAATTTATCAGACTTT |
| | ACACCAAGAAGTGATATGGAGTATGATTTTCTTAACATGGATATGGGTGTTTTTATTAATAAATATG |
| | GTCTTGAGGATTTTAATTTTGAACATGTTGTATATGGTGATGTTTCAAAAACTACATTAGGAGGTCT |
| | TCATTTGTTGATATCACAGTTTAGGCTTAGTAAAATGGGTGTTTTGAAAGCTGATGATTTTGTCACT |
| | GCTTCTGACACAACTTTGAGGTGCTGTACTGTTACTTATCTTAATGAACTTAGTTCAAAAGTTGTTT |
| | GTACTTATATGGATTTGTTGTTGGACGACTTTGTTACTATACTAAAGAGTTTAGATCTTGGTGTAAT |
| | ATCTAAAGTTCATGAAGTTATTATAGATAATAAACCTTATAGGTGGATGTTGTGGTGTAAAGATAAC |
| | CACTTGTCCACTTTTTATCCACAGTTGCAGTCTGCTGAATGGAAGTGTGGTTATGCTATGCCACAAA |
| | TTTATAAGCTTCAACGTATGTGTTTGGAACCTTGTAATTTGTGGCATTATGGTGCTGGTATTAAGTT |
| | GCCTAGTGGTATAATGTTAAATGTTGTTAAATACACTCAGCTTTGTCAATACCTAAATAGCACTACA |
| | ATGTGCGTACCTCATAATATGCGTGTTTTGCACTATGGTGCTGGTTCTGACAAAGGTGTGGCACCTG |
| | GTACAACTGTTTTAAAACGTTGGCTACCACCTGATGCAATAATCATTGATAATGATATCAATGATTA |
| | TGTTAGTGATGCAGATTTTAGCATTACAGGTGATTGTGCTACTGTTTATCTTGAAGATAAGTTTGAC |
| | TTACTTATTTCTGATATGTATGATGGTAGAATTAAATTTTGTGATGGTGAAAACGTCTCTAAAGATG |
| | GGTTTTTTACTTATCTTAATGGTGTTATTAGAGAAAAATTAGCTATTGGTGGTAGTGTTGCCATTAA |
| | GATTACAGAATATAGTTGGAATAAGTATCTTTATGAATTAATACAAAGATTTGCTTTTTGGACTTTG |
| | TTTTGCACGTCTGTTAACACATCCTCTTCGGAAGCTTTTCTTATTGGTATTAATTATTTAGGTGACT |
| | TTATTCAAGGTCCTTTTATAGCTGGTAACACTGTTCATGCTAATTATATATTTTGGCGTAATTCTAC |
| | TATTATGTCTTTGTCATACAATTCAGTTTTAGATTTAAGTAAGTTTGAATGTAAACATAAGGCCACT |
| | GTTGTTGTTACACTTAAAGATAATGATGTAAATGATATGGTTTTGAGTTTGATTAAGAGTGGTAGGT |
| | TGTTGTTACGCAATAATGGTCGTTTTGGTGGTTTTAGTAATCATTTAGTCTCAACTAAATGAAACTT |
| | TTCTTGATTTTGCTTGTTTTGCCCCTGGCCTCTTGCTTTTCCACATGTAATAGTAATGCTAATCTCT |
| | CTATGTTACAATTAGGTGTTCCTGACAATTCTTCAACTATTGTCACGGGTTTATTGCCAACTCATTG |
| | GATTTGTGCTAACCAGAGTACATCTGTTTACTCAGCCAATGGTTTCTTTTATATTGATGTTGGTAAT |
| | CACCGTAGTGCTTTTGCTCTCCATACTGGTTATTATGATGTTAATCAGTATTATATTTATGTTACTA |
| | ATGAAATAGGCTTAAATGCTTCTGTTACTCTTAAGATTTGTAAGTTTGGTAACACTACTTTTGATTT |
| | TTTAAGTAATTCTTCTAGTTCTTTTGACTGTATAGTTAATTTGTTATTTACAGAACAGTTAGGTGCG |
| | CCTTTGGGCATAACTATATCTGGTGAAACTGTGCGTCTGCATTTATATAATGTAACTCGTACTTTTT |
| | ATGTGCCAGCAGCTTATAAACTTACTAAACTTAGTGTTAAATGTTACTTTAACTATTCCTGTGTTTT |
| | TAGTGTTGTCAACGCCACCGTTACTGTGAATGTCACCACACATAATGGCCGTGTAGTTAACTACACT |
| | GTTTGTGATGATTGTAATGGTTATACTGATAACATATTTTCTGTTCAACAGGATGGCCGCATTCCTA |
| | ATGGTTTCCCTTTTAATAATTGGTTTTTGCTAACTAATGGTTCCACATTAGTGGACGGGGTCTCTAG |
| | ACTTTATCAACCACTCCGTTTAACTTGTTTATGGCCTGTACCTGGTCTTAAATCTTCAACTGGTTTT |
| | GTTTATTTTAATGCCACTGGTTCTGATGTTAATTGTAACGGCTATCAACATAATTCTGTTGCTGATG |
| | TTATGCGTTACAATCTTAACTTCAGTGCTAATTCTGTGGATAATCTCAAGAGTGGTGTTATAGTTTT |
| | CAAAACTCTACAGTACGATGTTTTGTTTTACTGTAGTAATTCCTCTTCGGGCGTTCTAGACACCACA |
| | ATACCTTTTGGCCCTTCCTCTCAACCTTATTACTGTTTTATAAATAGAACNATNAACACCACTCATG |
| | TTAGCACTTTTGTGGGTGTCTTACCACCCACTGTGCGTGAGATTGTTGTTGCTAGAACTGGTCAGTT |
| | TTATATTAATGGTTTTAAGTATTTTGATTTGGGTTTTATCGAAGCTGTTAATTTTAATGTCACAACT |
| | GCTAGTGCCACAGATTTTTGGACGGTTGCATTTGCTACTTTTGTTGATGTTTTGGTTAATGTTAGTG |
| | CAACTAAGATTCAAAACTTACTTTATTGCGATTCTCCATTTGAAAAGTTGCAGTGTGAGCACTTGCA |
| | GTTTGGATTGCAAGATGGTTTTTATTCTGCAAATTTTCTTGATGATAATGTTTTGCCTGAGACTTAT |
| | GTTGCACTCCCCATTTATTATCAACACACGGACATAAATTTTACTGCAACTGCATCTTTTGGTGGTT |
| | CTTGTTATGTTTGTAAACCACACCAGGTTAATTTATCTCTTAATGGTAACACTTCAGTGTGTGTTAG |
| | AACATCTCATTTCTCAATTAGGTATATTTATAACCGCGTTAAGAGTGGTTCACCAGGTGACTCTTCA |
| | TGGCACATTTATTTAAAGAGTGGCACTTGTCCATTTTCTTTTTCTAAGTTAAATAATTTTCAAAAGT |
| | TTAAGACTATTTGTTTTTCAACCGTCGCAGTGCCTGGTAGTTGTAATTTTCCACTTGAAGCCACCTG |
| | GCATTACACTTCTTATACTATTGTTGGTGCTTTGTATGTTACTTGGTCTGAAGGTAATTCTATTACT |
| | GGTGTACCTTATCCTGTCTNNNNNNNNNNNNNNNNNNNNNNNNNNNNNNNNNNNNNNNNNNNNNNNN |
| | NNNNNNNNNNNNNNNNNNNNNNNNNNNNNATTATACGTTCTTCAAACCAGTCACTTGCTGGTGGTAT |
| | TACATATGTTTCTAACTCTGGTAATTTACTTGGTTTTAAAAATGTTTCCACTGGTAACATTTTTATT |
| | GTGACACCATGTAACCAACCAGACCAAGTAGCTGTTTATCAACAAAGCATTATTGGTGCCATGACTG |
| | CTGTTAATGAGTCTAGATATGGCTTGCAAAACTTACTACAGTTACCTAACTTTTATTATGTTAGTAA |
| | TGGTAGTAACAATTGCACTACGGCCGTTATGACTTATTCTAATTTTGGTATTTGTGCTGATGGTTCT |
| | TTAATTCCTGTTCGTCTGCGCAATTCTAGTGATAATGGTATTTCAGCCATAATCACTGCTAATTTAT |
| | CTATTCCCTCTAACTGGACTACTTCAGTTCAAGTTGAGTACCTCCAAATTACTAGTACTCCAATAGT |
| | TGTTGATTGTGCTACTTATGTGTGTAATGGTAACCCTCGCTGTAAGAATCTACTTAAGCAGTATACT |
| | TCTGCTTGTAAAACTATTGAAGATGCCTTACGACTTAGTGCTCATTTGGAAACTAATGATGTTAGTA |
| | GTATGCTAACTTTCGATAGCAATGCTTTTAGTTTGGCTAATGTTACTAGTTTTGGAGATTATAACCT |
| | TTCTAGTGTTTTACCTCAGAGAAACATTCATTCAAGCCGTATAGCAGGACGTAGTGCTTTGGAAGAT |
| | TTGTTGTTTAGCAAAGTTGTTACATCTGGTTTGGGTACTGTTGATGTTGACTATAAGTCTTGTACTA |
| | AAGGTCTTTCTATTGCTGACCTTGCTTGTGCTCAGTACTACAATGGCATAATGGTTTTGCCAGGTGT |
| | TGCTGATGCTGAACGTATGGCCATGTACACAGGTTCTCTTATAGGTGGCATGGTGCTCGGAGGTCTT |
| | ACATCAGCAGCCGCCATACCTTTTTCTTTGGCACTGCAAGCACGACTTAATTATGTTGCTTTACAAA |
| | CTGATGTGCTTCAAGAAAATCAGAAAATTTTGGCTGCATCATTTAATAAGGCTATTAATAACATTGT |
| | TGCTTCTTTTAGTAGCGTTAATGATGCTATTACACAAACTGCAGAGGCTATACATACTGTTACTATT |
| | GCACTTAATAAGATTCAGGATGTTGTTAATCAACAGGGTAGTGCTCTTAACCATCTCACTTCACAAT |
| | TGAGACATAATTTTCAGGCCATTTCTAATTCAATTCAAGCTATTTATGACCGGCTTGATTCAATTCA |
| | AGCCGATCAACAAGTTGACAGATTAATTACTGGACGGCTTGCAGCTTTGAATGCATTTGTTTCCCAA |
| | GTTTTGAATAAATATACTGAAGTTCGTAGTTCCAGACGCTTAGCACAGCAGAAGATTAATGAATGTG |
| | TCAAATCACAATCTAATAGATATGGTTTTTGTGGCAATGGCACCCATATCTTTTCAATCGTCAACTC |
| | AGCTCCAGACGGTTTGCTTTTTCTTCATACTGTTTTGCTGCCAACCGACTACAAGAATGTAAAGGCG |
| | TGGTCGGGTATCTGTGTTGATGGCATTTACGGCTATGTTCTGCGTCAACCTAACTTGGTTCTTTATT |
| | CTGATAATGGTGTTTTTCGTGTAACTTCCAGGGTCATGTTTCAACCTCGTTTACCTGTTTTGTCTGA |
| | TTTTGTGCAAATATATAATTGTAATGTTACTTTTGTTAACATATCTCGTGTCAAGTTACATACTGTC |
| | ATACCTGACTACGTTGATGTTAATAAAACATTACAAGAGTTTGCACAAAATTTACCAAAGTATGTTA |
| | AGCCTAATTTTGACTTGACTCCTTTTAATTTAACATATCTTAATTTGAGTTCTGAGTTGAAGCAACT |
| | CGAAGCTAAAACTGCTAGTCTTTTCCAAACTACTGTTGAATTACAAGGTCTTATTGATCAGATTAAC |
| | AGTACATATGTTGACTTGAAGTTGCTTAATAGGTTTGAAAATTATATTAAATGGCCTTGGTGGGTTT |
| | GGCTCATTATTTCTGTTGTTTTTGTCGTATTGTTGAGTCTTCTTGTGTTTTGTTGTCTTTCTACAGG |
| | TTGTTGTGGTTGTTGCAATTGTTTAACTTCATCAATGCGAGGTTGTTGTGATTGTGGTTCAACTAAA |
| | CTTCCTTATTACGAATTTGAAAAGGTTCACGTTCAATAATGCCTTTCGGTGGCCTATTTCAACTTAC |
| | TCTTGAAAGTACTATTAATAAGAGTGTGGCTAATCTCAAATTACCACCTCATGATGTTACTGTCTTG |
| | CGTGACAATCTTAAACCTGTTACTACACTTAGTACTATCACTGCTTATTTGTTAGTTAGTTTGTTTG |
| | TCACTTATTTTGCTTTATTCAAACCTCTTACTGCTAGAGGTCGTGTTGCTTGTTTTGTTTTAAAACT |
| | ATTGACACTATTTGTCTATGTGCCTTTATTGGTTCTTTTTGGTATGTATCTTGACNNNNNNNNNNNN |
| | NNNNNNNCGCTGTTGTTTCGATTCATACATGTTGGCTATTATGCCTATCTCTATAAAAATTTTTCAT |
| | TTGTTTTGTTCAATGTTACTAAACTATGCTTCGTTTCAGGCAAGTGTTGGTATCTTGAACAATCATT |
| | TTATGAAAATCGTTTTGCTGCTATTTATGGTGGTGACCACTATGTCGTTTTAGGTGGTGAAACTATT |
| | ACTTTTGTTTCTTTTGATGACCTTTATGTTGCTATTAGAGGTTCTTGTGAAAAGAACCTACAACTTA |
| | TGCGTAAGGTTGACTTGTATAATGGTGCTGTCATTTACATTTTTGCCGAAGAGCCTGTTGTTGGTAT |
| | AGTCTACTCTTCTCAACTATACGAAGATGTTCCTTCGATTAATTGATGACAATGGCATTGTCCTCAA |
| | TTCTATTTTATGGCTCCTTGTTATGATATTTTTCTTTGTGTTGGCAATGACCTTTATTAAACTGATT |
| | CAATTGTGTTTTACTTGTCATTATTTTTTTAGTAGGACATTATATCAACCAGTTTATAAAATTTTTC |
| | TTGCTTACCAAGACTATATGCAAATAGCACCTGTTCCAGCTGAAGTATTAAATGTCTAAACTAAACG |
| | ATGTCTAATAGTAGTGTGCCTCTTTCAGAGGTTTATGTCCATTTACGTAACTGGAACTTTAGTTGGA |
| | ATTTAATTCTAACAGTTTTTATAGTTGTGTTGCAGTATGGGCATTATAAGTATAGCAGATTTCTTTA |
| | TGGTTTAAAGATGTCTGTTTTATGGTGTTTATGGCCACTTGTTCTAGCTTTGTCTATTTTTGACTGT |
| | TTTGTCAATTTTAATGTGGACTGGATCTTTTTTGGTTTTAGTATTCTTATGTCTATTATTACACTTT |
| | GTTTATGGGTTATGTATTTTGTTAATAGTTTCAGACTTTGGCGCCGTGTCAAAACTTTTTGGGCTTT |
| | TAATCCTGAAACTAATGCAATCATCTCTATCCAGGTTTATGGACATAATTACTACTTACCGGTGATG |
| | GCTGCACCTACAGGTGTTACATTAACACTTCTTAGTGGTGTACTTCTTGTTGATGGCCACAAGATTG |
| | CTACTCGTGTTCAAGTGGGTCAGTTGCCTAAATATGTAATAGTTGCTACACCTAGTACCACAATTGT |
| | TTGTGACCGTGTTGGTCGCTCTGTTAATGAAACAAGCCAGACTGGTTGGGCATTCTACGTCCGTGCT |
| | AAACATGGTGATTTTTCTGGTGTTGCCTCTCAGGAGGGTGTTTTGTCAGAAAGAGAGAAGTTGCTTC |
| | ATTTAATCTAAACTAAACAAAATGGCTAGTGTAAATTGGGCCGATGACAGAGCTGCTAGGAAGAAAT |
| | TTCCTCCTCCTTCATTTTACATGCCTCTTTTGGTTAGTTCTGATAAGGCACCATATAGGGTCATTCC |
| | CAGGAACCTTGTCCCTATTGGTAAGGGTAATAAAGATGAGCAGATTGGTTATTGGAATGTTCAAGAG |
| | CGTTGGCGTATGCGCAGGGGGCAACGTGTTGATTTGCCTCCTAAAGTTCATTTTTATTACCTAGGTA |
| | CTGGACCTCATAAGGACCTTAAATTCAGACAACGTCTTGATGGTGTTGTTTGGGTTGCTAAGGAAGG |
| | TGCTAAAACTGTTAATACCAGTCTTGGTAATCGCAAACGTAATCAGAAACCTTTGGAACCAAAGTTC |
| | TCTATTGCTTTGCCCCCAGAGCTCTCTGTTGTTGAGTTTGAGGATCGCTCTAATAACTCATCTCGTG |
| | CTAGCAGTCGTTCTTCAACTCGTAACAACTCACGAGACTCTTCTCGTAGTACTTCAAGACAACAGTC |
| | TCGCACTCGTTCTGATTCTAACCAGTCTTCTTCAGATCTTGTTGCTGCTGTTACTTTGGCTTTAAAG |
| | AACTTAGGTTTTGATAACCAGTCGAAGTCACCTAGTTCTTCTGGTACTTCCACTCCTAAGAAACCTA |
| | ATAAGCCTCTTTCTCAACCCAGGGCTGATAAGCCTTCTCAGTTGAAGAAACCTCGTTGGAAGCGTGT |
| | TCCTACCAGAGAGGAAAATGTTATTCAGTGCTTTGGTCCTCGTGATTTTAATCACAATATGGGGGAT |
| | TCAGATCTTGTTCAGAATGGTGTTGATGCCAAAGGTTTTCCACAGCTTGCTGAATTGATTCCTAATC |
| | AGGCTGCTTTATTCTTTGATAGTGAGGTTAGCACTGATGAAGTGGGTGATAATGTTCAGATTACCTA |
| | CACCTACAAAATGCTTGTAGCTAAGGATAATAAGAACCTTCCTAAGTTCATTGAGCAGATTAGTGCT |
| | TTTACTAAACCCAGTTCTATCAAAGAAATGCAGTCACAATCATCTCATGTTGTTCAGAACACAGTAC |
| | TTAATGCTTCTATTCCAGAATCTAAACCATTGGCTGATGATGACTCAGCCATTATAGAAATTGTCAA |
| | CGAGGTTTTGCATTAAATTGTTTTGTAATTCCAGTTGAATGTTTATTATTATTAGTTGCAACCCCAT |
| | GCGTTTAGCGCATGATAAGGGTTTAGTCTTACACACAATGGTAGGCCAGTGATAGTAAAGTGTAAGT |
| | AATTTGCTATCATATTAACATGTCTAGAGGAAAGTCAGAACTTTTTCTGTTTGTGTTGTTGGAGTAC |
| | TTAAAGATCGCATAGGCGCGCCAACAATGGAAGAGCCAACAACATATCTAAAAATGTTTTGTCTGGT |
| | ACTTGTTAATGATA |
| 9 | GenBank Accession No. MN369046.1 (Human coronavirus 229E strain |
| | HCoV_229E/Seattle/USA/SC9724/2018, complete genome) |
| | GTGCTGTGTGTTCTAGTCTAAGGGTTTCGTGTTCCGTCACGAGATTCCATTCTACAAACGCCTTACG |
| | CGAGGTTCTGTCTCGTGTTTGTGTGGAAGCAAAGTTTTGTCTTTGTGGAAACCAGTAACTGTTCCTA |
| | ATGGCCTGCAACCGTGTGACACTTGCCGTAGCAAGTGATTCTGAAATTTCTGCATATGGCTGTTCTA |
| | CTATTGCGCAAGCCGTCCGCCGTTATAGCGAGGCCGCTAGCAATGGTTTTAGGGCATGCCGATTTGT |
| | TTCATTAGATTTGCAGGATTGCATCGTTGGCATTGCAGATGATACATATGTTATGGGTCTGCATGGC |
| | AATCAGACGTTGTTTTGCAACATAATGAAATTTTCTGACCGTCCTTTTATGCTTCATGGGTGGTTGG |
| | TTTTTTCCAATTCAAATTACCTTTTGGAGGAATTTGATGTTGTCTTCGGTAAGAGAGGTGGTGGTAA |
| | TGTGACATACACTGACCAGTATCTCTGTGGCGCCGATGGCAAACCTGTTATGAGTGAAGATTTATGG |
| | CAGTTTGTTGACCATTTCGGTGAGAACGAAGAAATTGTCATCAATGGTCGTACTTACGTTTGTGCGT |
| | GGCTTACTAAGCGTAAGCCCTTAGATTACAAACGTCAGAACAACCTCGCCATTGAAGAGATTGAATA |
| | CGTGCATGGTGATGCTTTGCACACACTACGCAATGGTTCTGTTCTTGAAATGGCTAAGGAAGTGAAG |
| | ACATCTAGTAAAGTTGTGTTAAGCGATGCTCTTGACAAACTTTACAAAGTCTTTGGTTCTCCTGTTA |
| | TGACAAATGGTTCCAACATCCTAGAGGCCTTTACTAAACCTGTGTTTATTAGTGCATTAGTTCAATG |
| | TACTTGTGGTACCAAGTCTTGGTCTGTGGGTGATTGGACCGGTTTTAAATCCTCTTGTTGCAACTTG |
| | ATCAGTAATAAACTGTGTGTTGTGCCTGGTAATGTTAAACCTGGTGATGCTGTGATTACTACTCAGC |
| | AAGCTGGTGCTGGTGTTAAGTATTTTTGTGGCATGACTCTTAAGTTTGTTGCAAATATTGAAGGTGT |
| | CTCTGTTTGGAGAGTGATTGCTCTTCAGAGTGTTGATTGCTTTGTTGCTTCTTCCACTTTTGTAGAA |
| | GAGGAACATGTTAATAGAATGGACACATTCTGCTTCAATGTACGCAATAGTGTTACTGATGAGTGTC |
| | GTCTGGCCATGTTGGGTGCTGAAATGACTAGTAATGTCAGAAGACAAGTTGCTTCAGGTGTCATAGA |
| | CATTAGTACCGGCTGGTTTGATGTTTATGATGACATCTTTGCTGAAAGCAAACCATGGTTTGTTCGC |
| | AAGGCTGAAGACATTTTTGGCCCTTGTTGGTCTGCTCTTGTTTCTGCACTTAAACAACTTAAAGTCA |
| | CTACAGGTGAACTTGTGAGATTTGTTAAGTCTATTTGCAATTCAGCTGTTGCTGTCGTGGGTGGTAC |
| | TATACAAATTCTCGCTAGTGTGCCTGAGAAGTTTTTGAATGCGTTTGACGTGTTTGTCACAACTATT |
| | CAAACTGTCTTTGACTGTGCTGTTGAAACTTGTACTATTGCCGGTAAAGCATTTGACAAGGTTTTTG |
| | ACTATGTCTTGCTTGATAATGCGCTTGTAAAACTTGTCACCACAAAGCTTAAGGGTGTTCGTGAAGG |
| | TGGCCTTAATAAAGTTAAGTATGCAACAGTTGTTGTTGGTTCCACTGAAGAAGTTAAATCTTCACGT |
| | GTTGAACGTAGCAATGCTGTACTTACAATCGCCAACAATTATTCCAAACTTTCTGATGAAGGGTATA |
| | CTGTTGTAATTGGCGATGTGGCGTACTTTGCTAGTGACGGCTACTTCCGTCTTATGGCCAATCCAAA |
| | TAGTGTGTTGACTACTGCAGTCTATAAACCATTGGTTGATTTTAATGTGAATGTTATTGGTACTAGA |
| | CCTGAAAAATTTCCAACCACTGTGACTTGTGAAAATTTAGAGTCTGCTGTTTTGTTTGTTAATGACA |
| | AAATCACTGAATTCCAATTGGATTACAGTATTGATGTCATTGATAATGAAATAATTGTCAAACCTAA |
| | TATCAGCTTATGTGTTCCACTTTATGTGAGAGACTATGTTGACAAATGGGATGATTTTTGCAGACAA |
| | TATAGTAACGAGTCTTGGTTTGAGGATGATTACAAGGCTTTTATCAGTGTTTTGGATATCACTGATG |
| | CTGCTGTGAAAGCTGCAGAATCTAAAGCCTTCGTTGATACTATTGTTCCACCTTGCCCATCTATTTT |
| | GAAAGTTATAGATGACGGCAAAATATGGAATGGTGTTATTAAAAATGTTAACTATGTTAGAGACTGG |
| | CTTAAGTCGTTGAAGTTAAATCTCACACAACAGGGTTTGCTTGGAACATGTGCAAAGCGTTTTAAAC |
| | GTTGGCTTGGCATTTTGCTAGAGGCCTATAATGCGTTTTTAAACACTGTTGTTTCTACTGTTAAAAT |
| | TGGTGGTTTGACCTTTAAAACATATGCTTTTGATAAAACTTACATTGTGATACGTGATATCGTGTGT |
| | AAGGTTGAAAATAAAACAGAAGCAGACTGGATTGAACTTTTTCCACATAATGACAGGATTAAGTCCT |
| | TTAGTACTTTTGAGAGTGCTTACATGCCAATTGCAGACCCTACACATTTTGACATTGAAGAAGTTGA |
| | ACTTTTAGATGCAGAGTTTGTAGAACCAGGCTGTGGTGGTATTTTGTCAGTAATAGATGAGCACGTC |
| | TTTTATAAGAAGGATGGTGTTTATTATCCATCAAATGGTACTAACATTCTACCTGTTGCATTTACAA |
| | AAGCCGCTGGTGGTAAAGTTTCATTTTCTGATGACGTTGAAGTAAAAGACATTGAACCTGTTTACAG |
| | AGTCAAGCTTTGCTTTGAGTTTGAAGATGAAAAACTTGTAGATGTTTGTGAAAAGGCAATTGGCAAG |
| | AAAATTAAACATGAAGGTGACTGGGATAGCCTTTGTAAGACTATTCAATCAGCACTTTCTGTTGTTT |
| | CATGCTATGTAAATCTGCCTACTTATTACATTTATGATGAAGAAGGCGGTAATGACTTGAGTTTGCC |
| | CGTTATGATTTCTGAATGGCCTCTTTTTGAGTCAGATAAGGAAGAAGATGTTCAACAAGCTCAACAA |
| | GAAGCTACTCATATTGCTGAGGATGTTGTTGACCAAGTTGAAGAAGTCAATAGCATTTTTGACATTG |
| | AGACAGTGGATGTTAAACATGAGATGAGTCCTTTTGAAATGCCATTTGAAGAGTTAAATGGTTTAAA |
| | GATACTTAAACAATTGGATAACAACTGCTGGGTTAACTCAGTTATGTTACAAATACAATTAACTGGT |
| | ATACTTGATGGTGATTATGCTATGCAGTTTTTTAAAATGGGCCGAGTTGCCAAGATGATTGAACGCT |
| | GCTACACTGCTGAGCAATGTATACGTGGTGCTATGGGTGATGTTGGTTTGTGTATGTATAGACTTCT |
| | TAAAGACTTACACACTGGTTTTATGGTTATGGATTATAAATGTAGTTGTACCAGTGGTAGGCTTGAA |
| | GAATCGGGAGCTGTTTTGTTTTGTACGCCCACTAAGAAGGCGTTTCCTTATGGTACTTGTCTAAATT |
| | GTAACGCACCTCGCATGTGTACAATTAGGCAGTTACAAGGTACCATAATATTTGTGCAACAAAAACC |
| | AGAACCTGTTAACCCTGTTTCTTTTGTTGTTAAACCAGTCTGCTCATCAATTTTTCGTGGTGCTGTG |
| | TCTTGTGGTCATTACCAGACTAACATCTATTCACAAAATTTGTGTGTGGATGGTTTTGGTGTTAACA |
| | AGATTCAGCCCTGGACAAATGATGCACTTAATACTATTTGTATTAAGGATGCAGATTATAATGCAAA |
| | AGATGAAATATCTGTTACACCAATTAAAAACACAGTTGATACAATACCTAAGGAAGAATTTGTTGTT |
| | AAAGAGAAGTTGAACGCCTTCCTCGTTCATGGCAATGTAGTTTTCTACCAAGGTGATGTTGATACCG |
| | TTGTTAATGGTGTTGACTTTGACTTTATTGTAAATGCTGCTAATGAGAACCTTGCTCATGGTGGAGG |
| | ACTTGCCAAAGCTTTAGATGTGCACACTAAAGGTAAACTTCAACGTTTATCCAAAGAACACATTGGA |
| | TTAGCGGGTAAAGTAAGAGTTGGTACAGGAGTTATGGTTGAGTGTGATAGCCTTAGAATTTTTAATG |
| | TTGTTGGTCCACGCAAGGGTAAACATGAACGTGATTTACTCGTAAAAGCTTACAACACTATTAATAA |
| | CGAACAAGGCACACCTTTAACACCAATTTTGAGTTGTGGTATTTTTGGTGTCAAACTCGAAACTTCA |
| | TTGGAAGTTTTGCTTGATGTTTGTAATACAAAAGAAGTTAAAGTTTTTGTTTATACAGACACAGAAG |
| | TTTGTAAGGTTAAGGATTTTGTGTCTGGTTTAGTGAATGTTCAAAAAGTTGCACAACCTAAAATAGA |
| | ACAAAAACCAGTGTCCTTAATTAAAGTTGCACCCAAGCCTTACAGGGTAGATGGTAAATTTAGTTAC |
| | TTTACAGAAGACTTGTTGTGTGTCGCTGATGACAAACCCATTGTTTTGTTTACTGACTCTATGCTTA |
| | CTTTGGATGACCGTGGTTTGGCTCTAGACAATGCACTTAGTGGTGTGCTTAGTGCTGCTATTAAGGA |
| | TTGTGTTGACATAAATAAAGCTATACCTTCTGGTAATCTTATTAAGTTTGACATAGATTCTGTTGTT |
| | GTTTACATGTGTGTTGTGCCATCCGAAAAGGACAAACATTTAGATAATAATGTTCAACGATGCACAC |
| | GTAAGTTGAATAGACTTATGTGTGATATAGTTTGTACTATACCAGCTGACTACATCTTGCCATTGGT |
| | GTTGTCTAGTTTGACTTGTAATGTTTCTTTTGTAGGTGAACTTAAAGCTGCTGAAGCTAAAGTTATA |
| | ACTATAAAGGTGACAGAGGATGGTGTTAATGTTCATGATGTGACCGTGACAACAGATAAGTCATTTG |
| | AACAACAAGTTGGTGTTATTGCTGACAAGGACAAAGATCTTTCTGGTGCAGTACCCAGTGATCTTAA |
| | CACATCTGAATTGCTTACTAAAGCAATAGATGTTGATTGGGTCGAATTTTATGGCTTTAAAGATGCT |
| | GTTACTTTTGCAACAGTTGATCATAGTGCTTTTGCCTATGAAAGTGCTGTTGTTAATGGTATTAGAG |
| | TGTTAAAAACCAGTGATAATAATTGTTGGGTGAATGCTGTTTGTATTGCACTACAGTATTTGAAACC |
| | TCATTTTATTTCACAAGGTCTTGATGCTGCGTGGAATAAATTTGTTTTAGGCGATGTTGAAATTTTT |
| | GTTGCATTTGTTTACTATGTTGCAAGACTAGTGAAAGGTGACAAGGGTGATGCTGAAGACACTTTGA |
| | ATAAGTTGTCTAAGTATCTTGTTAATGAAGCTCAAGTTCAATTAGAACATTATAGTTCTTGTGTTGA |
| | ATGTGATGCTAAATTTAAAAACTCTGTTACATCTATCAATTCTGCTATAGTTTGTGCTAGTGTCAAA |
| | CGTGATGGTGTGCAAGTTGGTTATTGTGTCCATGGTATTAAGTACTATTCACGTGTTAAAAGTGTTA |
| | GAGGTAGAGCTATTATAGTCAGTGTCGAACAGCTTGAACCGTGTGTTCAGTCTAGACTTTTGAGTGG |
| | TGTTGCTTATACTGCTTTTTCTGGACCTGTTGATAAAGGTCATTATACTGTTTATGACACTGCAAAG |
| | AAATCAATGTATGATGGTGATCGTTTTGTTAAACATGATCTTTCTCTGCTGTCTGTCACATCAGTTG |
| | TTATGGTTGGTGATTATGTTGCACCTGTTAGTACAGTGAAACCTAAACCAGTCATTTCTCAACTTGA |
| | TGAAAATGCACAGAAGTTCTTTGATTTTGGTGATTTTTTGGTTCATAATTTTGTTATTTTTTTCACA |
| | TGGTTATTGAGTATGTTTACTTTGTGTAAAACTGCAGTAACTACATGTGATGTTAAAATAATGGCCA |
| | AAGCACCACAAAGGACGGGTGTTGTTTTAAAACGTAGTCTTAAATATAACTTAAAAGCGTCAGCAGC |
| | TGTTCTTAAATCTAAGTGGTGGCTGCTTGCTAAGTTTACGAAACTACTGTTACTTATATATACATTG |
| | TATTCAGTAGTTTTGTTTTGTGTACGTTTTGGACCGTTTGAGTTTTGTAGTGAGACTGTTAATGGTT |
| | ATGCTAAGTCAAACTTTGTCAAGGATGATTACTGTGATGGTTCATTGGGCTGCAAGATGTGTCTTTT |
| | TGGTTATCAAGAGCTAAGTCAATTTAGCCATTTGGATGTTGTGTGGAAGCATATAACAGACCCTTTG |
| | TTTAGTAATATACAACCTTTCATTGTTATGGTTTTGCTGCTTATATTTGGTGACAATTATTTGAGAT |
| | GCTTCTTGCTGTATTTTGTTGCTCAGATGATAAGCACAGTTGGTGTTTTTCTAGGTTATAAGGAAAC |
| | AAATTGGTTCTTGCACTTTATTCCATTTGATGTTATTTGTGATGAACTGTTTGTCACTGTTATTGTT |
| | ATTAAGGTTATTTCTTTTGTCAGACATGTGCTTTTTGGTTGTGAAAACCCAGATTGCATTGCGTGTT |
| | CTAAGAGTGCTAGACTTAAGAGATTCCCTGTTAACACAATTGTTAATGGTGTGCAACGTTTATTTTA |
| | TGTTAATGCAAATGGTGGTAGTAAGTTTTGTAAGAAACATAGATTTTTCTGTGTTGACTGTGACTCT |
| | TATGGTTATGGCAGCACGTTTATAACACCTGAAGTTTCTAGAGAACTTGGTAACATTACTAAAACAA |
| | ATGTGCAACCAACAGGTCCAGCGTATGTCATGATTGACAAAGTGGAGTTTGAAAATGGTTTTTACAG |
| | GTTGTATTCCGGTGAAACATTTTGGCGTTACAACTTTGATATAACTGAAAGCAAGTATTCTTGCAAA |
| | GAGGTTTTTAAAAATTGTAATGTTTTGGATGATTTCATCGTGTTTAACAATAATGGGACCAATGTAA |
| | CGCAGGTTAAAAATGCTAGTGTTTACTTTTCACAGTTGTTGTGTAGGCCCATTAAATTAGTTGACAG |
| | TGAACTTTTGTCCACTTTGTCAGTTGATTTTAATGGTGTCTTACACAAGGCATACATCGATGTACTA |
| | CGTAATAGCTTTGGTAAAGATCTTAATGCTAATATGTCTTTAGCCGATTGCAAGAGTGCTTTAGGCC |
| | TGTCTATTAGTGATCATGAATTTACTAGTGCTATTTCTAATGCACATCGTTGTGACGTGTTGTTATC |
| | TGATTTGTCATTTAACAACTTTGTCAGTTCGTATGCTAAACCTGAGGAAAAATTATCAGCTTATGAC |
| | TTGGCGTGTTGTATGCGTGCAGGTGCTAAGGTTGTTAATGCCAATGTTCTGACAAAGGACCAAACTC |
| | CTATTGTTTGGCATGCAAAGGATTTTAACAGTCTTTCTGCTGAAGGTCGTAAGTATATTGTAAAAAC |
| | TAGCAAAGCTAAGGGTTTGACTTTCTTGTTGACAATTAATGAAAACCAAGCTGTTACGCAAATACCT |
| | GCAACTAGCATTGTTGCTAAGCAAGGTGCTGGTGATGCTGGTCATTCATTAACATGGCTGTGGTTAC |
| | TGTGTGGTCTTGTGTGTTTGATTCAATTCTATTTGTGCTTTTTCATGCCCTATTTTATGCACGATGT |
| | AGTGAGTAGTTTTGAGGGTTATGATTTTAAGTATATAGAAAATGGTCAGTTGAAGAATTTTGAAGCG |
| | CCACTTAAATGTGTCAGAAACGTTTTTGAAAACTTTGAGGACTGGCATTATGCTAAGTTTGGCTTCA |
| | CACCTTTAAACAAGCAAAGCTGTCCTATTGTAGTTGGAGTTTCTGAAATTGTTAATACTGTCGCTGG |
| | CATTCCATCTAATGTGTATCTTGTTGGTAAAACTTTAATTTTTACACTACAAGCTGCTTTTGGTAAT |
| | GCTGGTGTTTGTTATGACATTTTTGGAGTCACAACACCTGAAAAGTGCATTTTTACTTCTGCTTGTA |
| | CTAGATTAGAAGGTTTGGGTGGTAACAATGTTTATTGTTATAACACAGAGCTTATGGAAGGTTCTTT |
| | GCCGTACAGTTCAATACAAGCTAATGCATATTATAAATATGATAATGGCAATTTTATTAAGTTGCCA |
| | GAAGTTATTGCACAAGGCTTTGGCTTTAGAACAGTACGTACTATTGCTACCAAATACTGCCGCGTAG |
| | GTGAATGTGTTGACTCCAATGCAGGTGTGTGTTTTGGCTTTGACAAGTGGTTTGTTAATGATGGACG |
| | TGTTGGCAATGGTTACGTTTGTGGTACTGGTTTGTGGAACCTTGTATTTAACATACTTTCCATGTTC |
| | TCATCTTCATTCTCTGTTGCTGCAATGTCAGGTCAAATTTTACTTAATTGTGCATTAGGTGCTTTTG |
| | CTATCTTTTGTTGTTTTCTTGTGACAAAGTTTAGACGCATGTTTGGTGACCTTTCTGTAGGTGTTTG |
| | TACTGTTGTTGTTGCTGTTTTGCTTAACAATGTCTCTTACATTGTAACTCAGAATCTAGTAACAATG |
| | ATTGCTTATGCCATATTGTATTTCTTTGCTACTAGAAGCTTACGCTATGCATGGATTTGGTGTGCTG |
| | CATATTTAATTGCGTATATTTCTTTTGCTCCATGGTGGTTGTGTGCTTGGTACTTTCTTGCTATGTT |
| | GACAGGTTTGTTACCTAGTTTGCTGAAGCTTAAAGTTTCGACAAATCTTTTCGAAGGTGACAAATTT |
| | GTAGGTACATTTGAAAGTGCTGCTGCAGGAACATTTGTCATTGACATTCGTTCTTATGAGAAACTTG |
| | CTAATAGCATCTCTCCAGAAAAGTTGAAAAGTTATGCTGCTAGCTATAATAGATACAAGTACTATAG |
| | TGGTAATGCAAATGAAGCTGATTACCGTTGTGCTTGTTATGCCTATTTAGCAAAAGCAATGTTGGAC |
| | TTTTCGCGTGATCATAATGACATCTTGTACACACCTCCGACTGTCAGTTATGGTTCTACATTACAGG |
| | CTGGTTTGCGCAAAATGGCACAACCATCTGGCTTTGTGGAAAAATGTGTTGTCCGTGTCTGCTATGG |
| | AAACACTGTGTTGAATGGGTTGTGGCTTGGTGATATTGTTTATTGCCCACGTCATGTTATCGCATCT |
| | AACACAACTTCTGCTATAGATTATGATCACGAATATAGTATTATGCGGTTGCATAATTTCTCTATAA |
| | TATCTGGTACAGCATTTCTTGGTGTTGTAGGTGCTACTATGCATGGAGTAACTCTTAAAATTAAGGT |
| | TTCACAGACTAACATGCACACACCTAGACACTCTTTTAGAACACTAAAATCTGGTGAAGGTTTTAAC |
| | ATCTTAGCATGCTATGATGGTTGTGCTCAGGGTGTTTTTGGTGTGAACATGAGAACTAATTGGACTA |
| | TCCGTGGTTCATTTATTAATGGTGCGTGTGGTTCTCCTGGCTATAATCTTAAAAATGGCGAGGTGGA |
| | ATTTGTTTATATGCATCAAATTGAACTCGGAAGTGGTAGTCATGTAGGTTCTAGCTTTGATGGTGTT |
| | ATGTATGGTGGTTTTGAAGACCAACCTAATCTTCAAGTTGAATCTGCAAACCAGATGTTAACAGTTA |
| | ATGTGGTTGCATTTCTTTATGCTGCTATATTGAATGGTTGCACATGGTGGCTTAAAGGTGATAAATT |
| | GTCTGTGGAGCATTATAATGAGTGGGCACAGGCTAATGGTTTCACAGTTATGAATGGTGAAGACGCT |
| | TTTTCCATTCTTGCTGCTAAAACTGGTGTCTGTGTGGAAAGATTACTTCATGCTATTCAAGTTTTGA |
| | ATAATGGATTTGGTGGTAAACAAATTTTGGGTTATTCTAGTCTCAATGATGAGTTCAGTATTAATGA |
| | AGTTGTCAAACAAATGTTTGGTGTTAACCTGCAAAGTGGTAAAACCACTAGTATGTTTAAATCCATA |
| | AGCTTGTTTGCTGGTTTCTTTGTCATGTTCTGGGCTGAATTATTTGTTTATACCACCACTATTTGGG |
| | TTAACCCTGGTTTTCTTACTCCTTTTATGATTTTGCTTGTTGCTTTGTCACTCTGTCTTACATTTGT |
| | TATTAAACATAAGGTTTTGTTTTTGCAAGTGTTTTTGTTGCCTTCAATTATTGTGGCTGCTATTCAA |
| | AACTGTGCTTGGGACTACCATATTACAAAGGTGTTGGCAGAGAAGTTTGATTATAATGTTTCTGTTA |
| | TGCAAATGGATATCCAGGGTTTTGTTAACATTTTTATTTGTCTTTTTGTTGCACTGTTGCATACTTG |
| | GCGCTTTGCTAAAGAGCGTTGTACACATTGGTGCACTTATTTGTTCTCACTCATTGCTGTTTTATAC |
| | ACTGCATTGTATAGTTATGACTACGTTAGTTTGCTGGTTATGCTACTTTGTGCAATTTCTAATGAAT |
| | GGTATATTGGTGCTATCATTTTTAGAATTTGTCGTTTTGGTGTTGCATTTTTACCAGTGGAATACGT |
| | GTCTTACTTTGATGGTGTTAAAACTGTGCTGTTGTTTTACATGTTGTTAGGCTTTGTTAGCTGTATG |
| | TACTATGGTTTGTTGTACTGGATTAACAGGTTCTGTAAGTGCACATTAGGTGTTTATGATTTCTGTG |
| | TTAGTCCAGCCGAATTTAAGTATATGGTTGCTAATGGTTTGAATGCACCAAATGGCCCTTTTGATGC |
| | ACTCTTTCTGTCTTTTAAACTAATGGGTATTGGTGGTCCTAGAACCATTAAAGTCTCTACTGTACAG |
| | TCTAAATTGACTGATCTTAAGTGCACAAACGTCGTTCTAATGGGCATTTTGTCTAACATGAACATAG |
| | CTTCTAATTCAAAGGAGTGGGCATATTGTGTTGAAATGCACAATAAAATAAATTTGTGTGACGACCC |
| | TGAAACTGCTCAAGAGTTATTGCTGGCGTTGTTGGCCTTTTTCTTGTCTAAGCATAGTGATTTTGGT |
| | CTTGGTGATCTTGTCGATTCTTATTTTGAGAACGACTCCATTTTGCAAAGTGTTGCATCTTCTTTTG |
| | TTGGTATGCCATCTTTTGTTGCATATGAAACAGCAAGACAAGAGTATGAAAATGCTGTTGCAAATGG |
| | TTCCTCACCACAAATAATCAAACAATTGAAGAAGGCTATGAATGTTGCAAAAGCTGAGTTTGACAGG |
| | GAGTCATCTGTTCAAAAGAAAATTAACAGAATGGCTGAACAAGCTGCTGCAGCTATGTACAAAGAAG |
| | CACGTGCTGTTAATAGAAAATCAAAAGTTGTAAGTGCAATGCATAGTTTACTCTTTGGCATGCTCCG |
| | ACGTTTGGACATGTCTAGTGTTGACAATATCCTTAATATGGCACGTAATGGTGTTGTCCCTCTTTCC |
| | GTTATCCCTGCTACTTCTGCAGCCAGGCTCGTCGTCGTAGTGCCAGATCATGATTCATTTGTGAAAA |
| | TGATGGTAGATGGTTTTGTGCACTACGCTGGTGTTGTTTGGATATTACAGGAAGTTAAGGATAATGA |
| | CGGTAAGAATGTGCATCTTAAAGATGTTACAAAGGAAAACCAGGAAATACTTGTTTGGCCTCTGATT |
| | TTGACTTGTGAACGTGTCGTTAAATTGCAGAACAATGAAATAATGCCAGGCAAGATGAAGGTCAAGG |
| | CCACCAAAGGTGAAGGTGATGGAGGCATTATTAGTGAAGGTAATGCTCTATACAACAATGAAGGTGG |
| | ACGTGCATTTATGTATGCATATGTGACTACGAAGCCTGGCATGAAGTATGTTAAATGGGAACATGAC |
| | TCTGGTGTGGTTACAGTTGAATTGGAACCACCTTGCAGATTTGTTATAGACACACCTACTGGACCCC |
| | AAATTAAGTATCTTTATTTTGTTAAGAATCTTAACAATTTAAGGAGAGGTGCTGTTTTGGGTTACAT |
| | TGGTGCCACTGTGAGATTGCAAGCTGGCAAACAGACTGAGTTTGTTTCAAACTCCCATTTATTAACA |
| | CATTGTGCTTTTGCTGTTGACCCAGCTGCAGCCTACCTTGATGCTGTTAAACAAGGCGCAAAACCTG |
| | TTGGCAATTGTGTAAAGATGTTGACTAATGGTTCTGGTAGTGGTCAGGCTATTACTAGTACCATTGA |
| | TTCTAACACCACGCAGGACACATATGGTGGCGCGTCTGTTTGTATTTATTGCAGAGCACATGTTGCA |
| | CATCCAACCATGGACGGTTTTTGTCAGTATAAAGGTAAGTGGGTACAAGTGCCTATAGGTACAAATG |
| | ACCCTATAAGATTTTGTCTTGAAAATACTGTTTGTAAAGTTTGTGGTTGTTGGCTTAATCATGGCTG |
| | TACATGTGACCGGACTGCTATCCAAAGTTTTGATAGCAGTTATTTAAACGAGTCAGGGGCTCTAGTG |
| | CCGCTCGACTAGAGCCCTGTAATGGTACAGACATAGATTACTGTGTCCGTGCATTTGACGTTTACAA |
| | TAAAGATGCGTCTTTTATCGGAAAAAATCTGAAGTCCAATTGTGTGCGCTTCAAGAATGTAGATAAG |
| | GATGACGCGTTCTATATTGTTAAACGTTGCATTAAGTCAGTTATGGACCACGAGCAGTCCATGTATA |
| | ACTTACTTAAAGGCTGTAATGCTGTTGCTAAGCATGATTTCTTTACTTGGCATGAGGGCAGAACCAT |
| | TTATGGTAATGTTAGTAGACAGGATCTTACTAAATACACCATGATGGATTTGTGCTTCGCTCTGCGT |
| | AACTTTGATGAAAAAGATTGTGGAGTTTTTAAGGAGATATTGGTTCTTACTGGTTGTTGTAATACTG |
| | ATTACTTTGAAATGAAGAATTGGTTTGACCCCATAGAAAATGAGGATATACACCGTGTGTATGCTGC |
| | TTTAGGCAAGGTAGTCGCAAATGCAATGCTTAAGTGTGTTGCTTTTTGCGACGAAATGGTGCTCAAA |
| | GGAGTTGTTGGTGTTTTGACCTTAGACAACCAAGATCTTAATGGGAATTTCTATGACTTCGGTGATT |
| | TTGTATTGTGTCCTCCTGGAATGGGAATACCCTACTGCACGTCATACTATGCTTATATGATGCCTGT |
| | TATGGGTATGACTAATTGTTTAGCTAGTGAGTGCTTTATGAAAAGTGACATCTTTGGTCAAGACTTC |
| | AAAACTTTTGATTTGTTGAAATATGATTTCACAGAACATAAGGAGGTTTTGTTTAACAAGTACTTTA |
| | AGTATTGGGGACAGGATTATCATCCTGATTGTGTTGATTGCCATGACGAGATGTGTATTTTGCATTG |
| | TTCAAATTTTAACACACTCTTCGCAACCACAATTCCAAACACAGCTTTTGGACCTCTGTGCAGAAAA |
| | GTGTTTATTGATGGTGTACCCGTAGTTGCTACTGCTGGTTACCACTTTAAACAATTAGGACTTGTGT |
| | GGAACAAAGATGTTAACACCCATTCTACCAGACTTACTATTACTGAACTCTTACAGTTTGTGACAGA |
| | TCCAACGCTTATAGTTGCGTCATCGCCTGCCTTGGTGGACAAACGCACTGTTTGTTTTTCTGTCGCT |
| | GCTTTGAGTACAGGATTAACATCCCAAACAGTAAAACCTGGCCATTTTAATAAGGAGTTTTATGACT |
| | TCTTACGTTCTCAAGGGTTTTTCGATGAGGGTTCAGAATTAACATTGAAGCATTTCTTTTTTACACA |
| | AAAGGGTGATGCTGCAATTAAAGATTTTGATTATTATCGTTACAACAGACCTACCATTCTGGATATT |
| | GGACAAGCTCGCGTAGCATATCAAGTGGCAGCTCGCTATTTTGACTGTTACGAGGGTGGCTGTATTA |
| | CATCTAGAGAGGTTGTTGTTACAAACCTTAATAAAAGCGCTGGTTGGCCCCTTAATAAGTTTGGTAA |
| | AGCTGGTTTATATTATGAGTCTATTAGTTATGAGGAACAAGATGCTATTTTTTTATTAACAAAGCGT |
| | AATATTCTCCCTACTATGACTCAGTTAAATCTTAAATATGCCATATCTGGTAAGGAACGTGCACGTA |
| | CAGTGGGTGGCGTCTCTTTATTAGCTACTATGACTACACGACAGTTTCATCAGAAATGTCTAAAATC |
| | CATAGTAGCTACCAGAAATGCCACTGTTGTTATCGGCACTACCAAGTTTTATGGCGGTTGGGATAAT |
| | ATGTTAAAGAACCTGATGGCCGATGTTGACGATCCTAAATTGATGGGATGGGACTATCCTAAGTGCG |
| | ATAGAGCTATGCCCTCAATGATTCGTATGTTGTCGGCTATGATTTTAGGTTCTAAGCATGTCACATG |
| | TTGTACGGCTAGTGATAAATTTTATAGACTTAGTAATGAGCTTGCTCAAGTTTTGACCGAGGTTGTT |
| | TATTCAAATGGTGGGTTTTATTTTAAACCTGGTGGTACAACTTCTGGTGATGCAACTACAGCCTACG |
| | CCAATTCTGTCTTTAATATATTTCAGGCTGTAAGTTCTAACATTAATTGCGTTTTGAGCGTTAACTC |
| | GTCAAATTGCAATAATTTTAATGTTAAGAAGTTACAGAGACAACTCTATGATAATTGCTATAGAAAT |
| | AGTAATGTTGATGAATCTTTTGTGGATGACTTTTATGGTTATTTGCAAAAGCATTTTTCTATGATGA |
| | TTCTTTCTGATGATGGTGTTGTGTGCTACAATAAAACTTATGCTGAACTTGGTTACATTGCTGATAT |
| | TAGTGCTTTTAAAGCCACTTTGTATTATCAGAATGGTGTGTTTATGAGTACAGCTAAGTGTTGGACT |
| | GAGGAAGATCTTTCTATAGGACCTCATGAATTTTGCTCACAGCACACTATGCAGATTGTAGATGAAA |
| | ATGGTAAGTATTATCTACCATATCCAGATCCTAGCCGTATTATTTCTGCTGGTGTTTTTGTGGATGA |
| | CGTCACTAAGACTGATGCTGTCATTCTTTTGGAACGCTATGTTTCTCTGGCTATAGATGCATACCCA |
| | TTGTCTAAGCATCCTAAACCTGAGTACAGGAAGGTGTTTTACGCATTGTTAGATTGGGTTAAACATC |
| | TCAACAAGACTCTTAACGAAGGTGTTTTGGAGTCTTTTTCTGTTACACTTTTAGATGAACATGAGTC |
| | TAAGTTTTGGGATGAAAGCTTTTATGCTAGTATGTATGAGAAGTCTACAGTATTACAAGCTGCTGGT |
| | CTTTGTGTAGTATGTGGCTCTCAAACAGTTCTAAGATGCGGTGATTGTTTACGCAGACCTATGTTGT |
| | GCACTAAGTGCGCCTATGATCATGTGTTTGGCACTGATCATAAGTTCATTTTAGCTATTACACCATA |
| | TGTTTGTAACACATCTGGCTGCAACGTAACTGACGTTACAAAACTGTATCTTGGAGGTTTGAATTAT |
| | TACTGTGTAGACCACAAACCACATCTTTCATTCCCACTGTGTTCAGCTGGTAATGTCTTTGGTTTGT |
| | ACAAAAGTTCTGCTTTGGGTTCCATGGACATTGATGTCTTTAACAAACTTTCTACCTCTGATTGGTC |
| | TGACATTCGCGACTACAAGCTGGCTAATGATGCAAAAGAGTCACTAAGGTTGTTTGCAGCTGAAACG |
| | GTCAAGGCTAAAGAGGAAAGTGTTAAGTCATCATACGCTTATGCTACCCTAAAGGAGATTGTAGGTC |
| | CTAAGGAACTTTTGCTTTTATGGGAAAGTGGAAAAGCCAAACCACCGTTAAACCGTAATTCTGTTTT |
| | TAGATGOTTCCAAATTACAAAAGACTCCAAGTTTCAAGTTGGTGAGTTTGTGTTTGAGAAAGTAGAT |
| | TATGGTTCTGATACGGTTACTTACAAATCCACTGCTACTACTAAGTTAGTACCAGGTATGTTATTTA |
| | TTTTGACTTCTCATAATGTTGCTCCACTTAGAGCGCCAACAATGGCAAACCAGGAGAAATATTCTAC |
| | CATTTACAAGTTGCACCCATCATTTAATGTTAGTGATGCTTATGCAAATCTTGTACCTTATTACCAA |
| | CTTATTGGCAAACAGCGTATCACCACAATACAGGGTCCTCCTGGTAGTGGAAAATCGCATTGCTCTA |
| | TTGGTATTGGTGTGTATTACCCTGGAGCGAGGATCGTGTTCACTGCTTGTTCTCACGCTGCTGTTGA |
| | TTCGCTTTGTGCAAAAGCTGTCACAGCCTATAGTGTTGATAAGTGTACACGTATTATTCCTGCACGT |
| | GCCAGAGTTGAGTGTTATAGTGGTTTTAAACCTAATAACACTAGTGCACAATACGTGTTTAGTACTG |
| | TTAATGCGTTACCTGAAGTTAATGCAGACATTGTTGTCGTGGATGAGGTGTCTATGTGCACTAACTA |
| | TGACTTGTCTGTGATTAACCAGCGTATATCATATAAACACATTGTATATGTTGGTGATCCTCAACAG |
| | CTTCCAGCTCCTAGAGTTCTTATCTCTAAAGGTGTTATGGAACCAATTGACTATAATGTTGTGACAC |
| | AACGTATGTGTGCTATAGGACCCGATGTCTTTTTACACAAGTGTTACAGATGTCCTGCTGAAATAGT |
| | TAACACTGTTTCAGAGCTTGTTTATGAAAACAAGTTTGTACCTGTCAAAGAAGCTAGTAAGCAGTGC |
| | TTCAAAATCTTTGAACGCGGTAGTGTTCAGGTAGACAATGGCTCCAGTATAAATAGGCGTCAACTTG |
| | ATGTTGTTAAGCGATTTATACATAAAAACTCCACATGGAGCAAGGCTGTGTTTATTTCACCTTACAA |
| | TAGTCAAAATTATGTAGCTGCCAGGCTTTTAGGTTTACAAACTCAGACAGTGGATTCTGCTCAAGGT |
| | AGTGAATATGACTATGTTATATTCGCACAGACATCAGATACTGCTCATGCCTGTAATGCCAATCGTT |
| | TTAACGTTGCCATTACTAGAGCAAAGAAAGGTATTTTCTGTATTATGTCTGACAGAACTTTGTTTGA |
| | TGCACTTAAGTTCTTTGAAGTTACTATGACAGATTTACAGTCTGAAAGCAGTTGTGGTTTGTTTAAG |
| | GATTGTGCACGTAACCCTATTGATTTACCACCAAGTCATGCCACTACTTATTTGTCATTGTCTGATA |
| | GATTTAAGACTAGTGGTGACTTGGCTGTTCAAATAGGTAACAACAATGTTTGTACCTATGAACATGT |
| | GATTTCATATATGGGTTTCAGGTTTGATGTTAGCATGCCTGGTAGTCATAGTTTGTTCTGTACTAGA |
| | GACTTTGCCATGCGTCATGTCAGAGGTTGGTTAGGAATGGATGTGGAAGGTGCACATGTCACAGGTG |
| | ACAATGTTGGCACTAATGTACCTCTACAAGTTGGTTTTTCCAATGGTGTTGATTTTGTAGCTCAACC |
| | TGAAGGTTGTGTTTTAACAAACACTGGCAGTGTTGTAAAACCTGTTCGTGCTCGTGCACCTCCTGGA |
| | GAACAATTTACTCACATTGTACCTCTGTTACGCAAGGGACAACCTTGGAGTGTGTTGAGAAAACGTA |
| | TTGTTCAAATGATAGCAGATTTTCTTGCTGGCTCATCTGATGTATTGGTGTTTGTGCTTTGGGCTGG |
| | CGGTTTAGAGTTGACCACTATGCGTTATTTTGTTAAGATTGGAGCTGTTAAACATTGCCAATGTGGT |
| | ACTGTTGCAACATGCTACAATTCTGTTAGTAATGACTATTGTTGCTTTAAACATGCATTGGGCTGTG |
| | ACTATGTTTATAACCCATATGTCATAGATATTCAACAATGGGGTTATGTTGGTTCACTCTCCAATAA |
| | TCACCATGCAATTTGTAATGTTCATAGAAATGAACACGTTGCTTCTGGTGATGCTATTATGACTAGA |
| | TGTTTGGCTGTGTATGATTGCTTTGTTAAGAATGTGGATTGGTCAATTACCTACCCTACGATAGCTA |
| | ATGAAAATGCCATAAACAAGGGCGGTCGCACTGTGCAGAGTCATATTATGCGTGCTGCTATTAAATT |
| | GTACAACCCTAAAGCAATCCATGACATTGGTAATCCTAAGGGTATTCGTTGTGCTGTAACTGATGCC |
| | AAGTGGTATTGTTATGACAAGAACCCTATTAATTCTAATGTGAAAACATTGGAGTATGATTACATGA |
| | CACATGGCCAAATGGAAGGCTTGTGTTTATTTTGGAATTGTAATGTGGATATGTACCCTGAATTCTC |
| | AATTGTTTGCAGGTTTGACACACGTACACGATCTACATTGAACCTCGAAGGTGTAAATGGTGGTTCA |
| | TTGTATGTCAATAACCATGCGTTTCACACTCCTGCTTATGATAAACGTGCTATGGCTAAATTGAAAC |
| | CAGCACCGTTTTTCTACTATGACGACGGTTCATGTGAGGTTGTTCACGATCAAGTTAACTATGTTCC |
| | TTTGAGAGCCACTAATTGCATTACCAAGTGTAATATTGGTGGTGCTGTATGTTCTAAGCACGCTAAT |
| | CTCTATAGAGCATATGTTGAGTCATATAATATTTTTACTCAAGCTGGTTTTAATATTTGGGTTCCTA |
| | CCACGTTTGATTGTTATAATTTGTGGCAGACATTCACAGATGTCAATTTACAAGGTTTAGAGAACAT |
| | TGCTTTTAACGTTGTTAATAAAGGTTCATTTGTTGGTGCTGACGGTGAATTACCAGTAGCCATTAGT |
| | GGTGATAAAGTGTTCGTACGTGATGGTAACACTGATAATTTAGTCTTTGTTAACAAAACATCACTGC |
| | CTACAAATATAGCATTTGAACTTTTTGCTAAGAGGAAGGTTGGTTTAACACCACCTCTCAGTATTCT |
| | CAAAAACCTTGGTGTTGTCGCCACATATAAGTTTGTCTTGTGGGATTATGAAGCTGAGCGTCCCTTG |
| | ACAAGCTTTACTAAGTCTGTTTGTGGTTATACAGACTTTGCAGAAGATGTTTGTACTTGTTACGATA |
| | ATAGTATACAAGGTTCATACGAACGTTTTACTCTCTCAACTAATGCTGTGTTATTCTCTGCTACTGC |
| | TGTGAAAACAGGTGGTAAGAGTTTGCCGGCTATTAAACTGAATTTTGGAATGCTTAACGGTAATGCA |
| | ATTGCTACTGTCAAATCAGAAGATGGTAACATAAAAAATATTAACTGGTTTGTTTACGTACGCAAAG |
| | ATGGTAAACATGTTGATCATTATGATGGTTTTTATACCCAAGGTCGTAATTTACAAGACTTTTTGCC |
| | TCGCAGCACAATGGAAGAAGACTTTTTGAACATGGATATAGGCGTGTTTATTCAAAAGTATGGTCTA |
| | GAGGATTTCAACTTCGAGCACGTTGTGTATGGCGATGTTTCAAAAACTACTCTAGGCGGTTTACACT |
| | TGTTGATTTCACAAGTACGTCTGAGTAAAATGGGCATCTTAAAGGCAGAGGAGTTTGTGGCAGCATC |
| | TGACATAACACTCAAATGTTGTACTGTGACTTATCTTAATGATCCTAGTTCTAAGACTGTTTGTACT |
| | TACATGGATTTGTTGTTGGATGATTTTGTTTCTTTATTGAAGTCTTTGGATTTGACTGTTGTATCCA |
| | AGGTTCATGAGGTCATAATTGACAACAAACCATGGAGATGGATGCTATGGTGTAAAGATAATGCCGT |
| | TGCTACATTCTATCCTCAGTTGCAGAGTGCAGAATGGAAATGCGGGTATTCTATGCCTGGTATTTAT |
| | AAGACACAACGTATGTGCTTAGAACCATGTAATTTGTATAATTATGGTGCAGGTTTGAAGTTGCCCA |
| | GTGGCATTATGTTCAATGTTGTTAAATACACTCAATTGTGTCAATATTTTAACAGTACCACGTTATG |
| | TGTTCCTCATAATATGAGAGTGTTACACTTGGGTGCTGGCTCTGATTATGGTGTTGCACCAGGAACT |
| | GCTGTTCTTAAAAGGTGGTTGCCGCACGACGCAATTGTTGTTGACAATGATGTTGTTGACTATGTGA |
| | GTGATGCTGATTTTAGTGTTACTGGTGATTGTGCAACCGTTTATTTGGAAGACAAGTTTGACTTGTT |
| | AATTTCTGATATGTACGATGGTAGGACAAAGGCAATTGATGGTGAAAATGTTTCAAAAGAAGGATTT |
| | TTCACTTACATCAATGGTTTCATTTGTGAAAAACTTGCCATCGGAGGTTCAATTGCTATTAAAGTAA |
| | CAGAGTATAGCTGGAATAAGAAATTGTATGAACTTGTACAAAGATTTTCTTTTTGGACTATGTTTTG |
| | CACTTCTGTTAATACGTCATCATCAGAAGCCTTTGTTGTCGGAATTAACTATCTTGGTGATTTCGCA |
| | CAAGGACCTTTTATAGATGGTAACATAATACATGCAAATTATGTATTTTGGCGTAACTCCACTGTTA |
| | TGAGTTTGTCCTACAACTCTGTTTTAGACCTGAGTAAATTTAATTGCAAACACAAAGCTACTGTTGT |
| | TGTGCAATTAAAGGATAGTGATATTAATGACATGGTGCTTAGTCTTGTTAGGAGTGGTAAGTTGCTT |
| | GTAAGGGGTAATGGCAAGTGTTTGAGTTTTAGTAATCATTTAGTCTCAACTAAATAAAATGTTTGTT |
| | TTACTTGTTGCATATGCCTTGTTGCATATTGCTGGTTGTCAAACTACAAATGGGACGAACACTAGTC |
| | ACTCTGTTTGCAACGGCTGTGTTGGTCATTCGGAAAATGTATTTGCTGTTGAGAGTGGTGGTTATAT |
| | ACCCTCCAACTTTGCATTTAATAATTGGTTCCTCTTAACTAATACCTCATCTGTTGTAGATGGTGTT |
| | GTGAGGAGTTTTCAGCCTTTGTTGCTTAATTGCTTATGGTCTGTTTCTGGCTCGCAGGTTATTACTG |
| | GTTTTGTCTATTTTAATGGTACTGGGAGAGGTGCTTGTAAAGGTTTTTATTCAAATGCTTCGTCTGA |
| | TGTCATACGTTACAACATCAATTTTGAAGAAAACCTTAGACGTGGAACCATTTTGTTTAAAACATCT |
| | TATGGTGCTGTTGTGTTTTATTGTACCAACAACACTTTGGTTTCAGGTGATGCGCACATACCATCTG |
| | GTACAGTTTTGGGCAATTTTTATTGCTTTGTAAATACTACTATTGGCAATGAAACTACGTCTGCTTT |
| | TGTGGGTGCACTACCTAAGACAGTTCGTGAGTTTGTTATTTCACGCACAGGACATTTTTATATTAAT |
| | GGCTATCGCTATTTCTCTTTAGGTAATGTAGAAGCCGTTAATTTCAATGTCACTAATGCTGCAACCA |
| | CTGTTTGTACTGTTGCGTTAGCTTCTTATGCTGACGTTTTGGTTAATGTTTCACAAACTGCTATTGC |
| | TAATATAATTTATTGCAATTCTGTTATTAACAGACTGAGATGTGACCAGCTGTCCTTTGATGTACCA |
| | GATGGTTTTTATTCCACAAGCCCTACTCAACCCGTTGAGCTACCTGTGTCTATTGTGTCGCTACCTG |
| | TTTATCACAAACACACGTTTATTGTGTTGTATGTTAACTTTGAACATCAGCGTGGCCCTGGCAAGTG |
| | CTATAACTGTCGTCCTGCTGTTATTAATATTACACTGGCCAATTTTAATGAAACTAAAGGGCCCTTG |
| | TGTGTTGACACATCACACTTCACTACCCAATTCGTCGACAATGTCAAGCTTGCTAGGTGGAGTGCTA |
| | GTATTGACACGGGAAATTGCCCTTTTTCTTTTGGCAAAGTTATTAACTTTGTTAAATTTGGCAGTGT |
| | ATGTTTTTCGCTAAAGTATATACCCGGTGGTTGCGCAATGCCTATAATGGCTAATTTGGTTAATTAT |
| | AAATCTCATAATATAGGCTCATTGTATGTTTCTTGGAGTGATGGTGATGTAATTACTGGCGTCCCGA |
| | AACCTGTTGAGGGTGTTAGTTCCTTTATGAATGTTACATTGAATAAATGTACTAAATATAACATTTA |
| | TGATGTATCTGGTGTGGGTGTTATTCGCATTAGCAATGACACCTTTCTTAATGGAATTACGTACACA |
| | TCAACTTCAGGTAACCTTCTGGGTTTTAAAGATGTTACTAATGGCACCATCTACTCTATCACTCCCT |
| | GTAACCCACCAGATCAGCTTGTTGTTTATCAGCAAGCTGTTGTTGGTGCTATGTTGTCTGAAAACTT |
| | TACTAGTTACGGCTTTTCTAATGTTGTAGAAATGCCGAAATTTTTCTATGCGTCCAATGGCACTTAT |
| | AATTGCACAGACGCTGTTTTAACTTATTCTAGTTTTGGCGTTTGTGCAGATGGTTCTATAATTGCTG |
| | TTCAACCACGTAATGTTTCATATGATAGTGTTTCAGCTATTGTCACAGCTAATTTGTCTATACCCTC |
| | CAATTGGACCACTTCGGTCCAGGTTGAGTATTTACAAATTACAAGTACACCTATCGTAGTTGATTGC |
| | TCCACTTATGTTTGCAATGGTAATGTGCGCTGTGTTGAATTGCTTAAGCAGTACACTTCTGCTTGTA |
| | AAACTATTGAAGACGCCTTAAGAAATAGCGCCATGCTGGAGTCTGCAGATGTTAGTGAGATGCTCAC |
| | TTTTGACAAGAAAGCGTTTACACTTGCTAATGTTAGTAGTTTTGGTGACTACAACCTTAGCAGTGTT |
| | ATACCTAGCTTGCCCAGAAGTGGTAGTAGAGTGGCTGGTCGCAGTGCCATAGAAGACATACTTTTTA |
| | GCAAACTTGTTACGTCTGGACTTGGCACTGTAGACGCAGACTACAAAAAGTGTACTAAGGGTCTTTC |
| | CATTGCTGACTTGGCTTGTGCCCAATATTATAATGGCATTATGGTTTTGCCTGGCGCCGCTGATGCT |
| | GAACGTATGGCCATGTATACAGGTTCTTTAATTGGTGGAATTGCTTTAGGAGGTCTTACATCAGCCG |
| | CTTCAATACCATTTTCATTAGCAATTCAGTCACGTTTAAATTATGTTGCATTGCAGACTGATGTTTT |
| | ACAAGAAAATCAGAGAATTCTTGCTGCATCTTTTAATAAAGCAATGACCAACATAGTAGATGCCTTT |
| | ACTGGTGTTAATGATGCTATTACACAAACTTCACAAGCTCTACAAACAGTTGCTACTGCACTTAATA |
| | AGATCCAGGATGTTGTTAATCAACAAGGCAACTCATTGAACCATTTAACTTCTCAGTTGAGGCAGAA |
| | TTTTCAAGCCATTTCTAGCTCTATTCAGGCTATCTATGACAGACTTGATATTATTCAGGCTGATCAA |
| | CAAGTAGATAGGCTGATTACTGGTAGACTGGCTGCTCTGAATGTATTCGTTTCTCATACATTGACTA |
| | AGTACACTGAAGTTCGTGCTTCCAGACAGCTTGCACAACAAAAAGTGAATGAGTGTGTCAAATCCCA |
| | GTCTAAGCGGTATGGCTTCTGTGGAAATGGCACGCACATTTTCTCACTTGTTAATGCTGCTCCTGAG |
| | GGGCTTGTTTTTCTCCATACTGTCTTGCTGCCGACACAATATAAGGATGTTGAAGCGTGGTCTGGGT |
| | TGTGCGTTGATGGTATAAACGGTTATGTGTTGAGACAACCTAATCTTGCTCTTTACAAAGAAGGCAA |
| | TTATTATAGAATTAGATCTCGCATAATGTTTGAACCACGTATTCCTACCATAGCAGATTTTGTCCAA |
| | ATTGAAAATTGCAATGTCACATTTGTTAACATTTCTCGCTCTGAGTTGCAAACTATTGTGCCAGAGT |
| | ATATTGATGTTAATAAGACGCTGCAAGAATTAAGTTACAAATTGCCAAATTACACTGTTCCAGACCT |
| | AGTTGTTGAACAGTACAACCAGACTATTTTGAATTTGACCAGTGAAATTAGCACCCTTGAAAATAAA |
| | TCTGCGGAGCTTAATTACACTGTTCAAAAACTGCAAACTTTGATCGACAACATAAATAGCACATTAG |
| | TTGACTTAAAGTGGCTCAACCGTGTTGAGACTTATATCAAGTGGCCTTGGTGGGTGTGGTTGTGCAT |
| | TTCAGTCGTGCTCATCTTTGTGGTGAGTATGTTGCTATTATGTTGTTGTTCTACTGGTTGCTGCGGC |
| | TTCTTTAGTTGTTTTGCATCTTCTATTAGAGGTTGTTGTGAATCAACTAAACTTCCTTATTACGACG |
| | TTGAAAAGATCCACATACAGTAATGGCTCTAGGTTTGTTCACATTGCAATTTGTGTCTGCTGTTAAT |
| | CAATCTCTTACCAAGTCGAAAGTTAGTGCTGAAGTTTCACGACAGGTTATCCAAGACGTGAAAGATG |
| | GCACTGTTACCTTCAACTTGCTAGCGTATACACTAATGAGCCTCTTTGTTGTGTATTTTGCTTTATT |
| | TAAAGCAAGATCACACCGTGGTAGATCTGCTCTTATAGTGTTTAAAATTTTAATCCTTTTCGTTTAT |
| | GTGCCATTGCTGTATTGGTCTCAAGCATATATTGACGCAACGTTGATTGCTGTAATTTTGCTTGGAA |
| | GATTTTTCCATACAGCTTGGCACTGCTGGCTCTACAAGACATGGGATTTCATTGTCTTCAATGCAAC |
| | CACACTTTGCTATGTGCAAGGTAAGTGTTGGTTTCTTGAAAATAAGGCTCTGAAACCATTCGTCTGT |
| | TTTTACGGAGGGGACCAATTCCTTTACATAGGTGACAGAATTGTTACTTATCTCTCAACTAACGATT |
| | TGTACGTTGCTCTTAGAGGACGTATTGATAAAGACCTCAGCCTTTCTAGAAAGGTTGAGTTATATAA |
| | CGGTGAATGTGTATACTTGTTTTGTGAACACCCAGCTGTTGGAATAGTCAATACAGATTTCAAATTA |
| | GAAATCCACTAAGATGTTCCTTAAGCTAGTGGATGATCATGCTTTGATTGTTAATGTACTACTCTGG |
| | TGTGTGGTGCTTATAGTGATACTACTAGTGTGTATTACAATAATTAAACTAATTAAGCTTTGTTTCA |
| | CTTGCCATATGTTTTGTAATAGAACAGTTTATGGCCCCATTAAAAATGTGTACCATATTTACCAATC |
| | ATATATGCACATAGACCCTTTCCCTAAACGAGTTATTGATTTCTAAACTAAACGACAATGTCAAACG |
| | ACAATTGTACGGGTGACATTGTCACCCATTTGAAGAATTGGAATTTTGGTTGGAATGTTATTCTAAC |
| | CATATTCATTGTTATTCTTCAGTTTGGACACTATAAATACTCCAGATTGTTTTATGGTTTGAAGATG |
| | CTTGTACTGTGGCTTCTTTGGCCACTTGTACTTGCTTTGTCAATCTTTGACACCTGGGCTAATTGGG |
| | ACTCTAATTGGGCCTTTGTTGCATTTAGCTTCCTTATGGCCGTATCAACACTCGTTATGTGGGTGAT |
| | GTACTTTGCAAATAGTTTCAGACTTTTCCGACGTGCTCGAACTTTTTGGGCATGGAATCCTGAGGTT |
| | AATGCAATCACTGTCACAACCGTGTTGGGACAGACATACTATCAACCCATTCAACAAGCTCCAACAG |
| | GCATTACTGTGACCTTGTTGAGCGGCGTGCTTTACGTTGACGGACATAGATTGGCTTCAGGTGTTCA |
| | GGTTCATAACCTACCTGAATACATGACAGTTGCCGTGCCTAGTACTACTATAATTTATAGTAGAGTC |
| | GGAAGGTCCGTAAATTCTCAAAATAGCACAGGCTGGGTTTTCTACGTACGAGTAAAACACGGTGATT |
| | TTTCTGCAGTGAGCTCCCCCATGAGCAACATGACAGAAAACGAAAGATTGCTTCATTTTTTCTAAAC |
| | TGAACGAAAAGATGGCTACAGTCAAATGGGCTGATGCATCTGAACCACAACGTGGTCGTCAGGGTAG |
| | AATACCTTATTCTCTTTATAGCCCTTTGCTTGTTGATAGTGAACAACCTTGGAAGGTGATACCTCGT |
| | AATTTGGTACCCGTCAACAAGAAAGACAAAAATAAGCTTATAGGCTATTGGAATGTTCAAAAACGTT |
| | TCAGAACTAGAAAGGGCAAACGGGTGGATTTGTCACCCAAGTTACATTTTTATTATCTTGGCACAGG |
| | ACCTCATAAAGATGCAAAATTTAGAGAGCGTGTTGAAGGTGTTGTCTGGGTTGCTGTTGATGGTGCT |
| | AAAACTGAACCTACAGGTTACGGTGTTAGGCGCAAGAATTCAGAACCAGAGATACCACACTTCAATC |
| | AAAAGCTCCCAAATGGTGTTACTGTTGCTGAAGAACCTGACTCCCGTGCTCCTTCCCGTTCTCAGTC |
| | AAGGTCTCAGAGTCGTATTCAATCTCGGAATCCTTCAAGTGACAGAAACCATAACAGTCAGGATGAC |
| | ATCATGAAGGCAGTCGCTGCGGCTCTTAAATCTTTAGGTTTTGACAAGCCTCAGGAAAAAGACAAAA |
| | AGTCAGCGAAAACGGGTACCCCTAAGCCTTCTCGTAATCAGAGTCCTGCTTCTTCTCAATCTGCTGC |
| | CAAGATTCTTGCTCGTTCTCAGAGTTCTGAAACAAAAGAACAAAAGCATGAAATGCAAAAGCCACGG |
| | TGGAAAAGACAGCCTAACGATGATGTGACATCTAATGTCACACAATGTTTTGGCCCCAGAGACCTTG |
| | ACCACAACTTTGGAAGTGCAGGTGTTGTGGCCAATGGTGTTAAAGCTAAAGGCTACCCACAATTTGC |
| | TGAGCTTGTGCCGTCTACAGCTGCTATGCTTTTTGATAGTCACATTGTTTCCAAAGAGTCAGGCAAC |
| | ACTGTGGTCTTGACTTTCACCACTAGAGTGACTGTGCCCAAAGACCATCCACACTTGGGTAAGTTTC |
| | TTGAGGAATTAAATGCATTCACTAGAGAAATGCAACAACAGCCTCTTCTTAACCCTAGTGCACTAGA |
| | ATTCAACCCATCCCAAACTTCACCTGCAACTGTTGAACCAGTGCGTGATGAAGTTTCTATTGAAACT |
| | GACATAATTGATGAAGTCAACTAAACATGCTACTGTGTTGAAATTCAGGCTTTAGTTGGAATTTTGC |
| | TTTTGCTCTTGTTTTTATTATCTTTCTTTTATGAATATATTTCTTTCCTCTGATCTACGTAATGGTA |
| | CGATCAGAGCTGCTTTTAATTAACATGATCCCTTGCTTTGGCTTGACAAGGATCTAGTCTTATACAC |
| | AATGGTAAGCCTGTGGTAGTAAAGGTATAAGAAATTTGCTACTATGTTACTGAACCTAGGTGAACGC |
| | TAGTATAACTCATTACAATTGTGCTGGAGTAATCAAAGATCGCATTGACGAGCCAACAATGGAAGAG |
| | CCAGTCATTTGTCTTGAGACCTATCTAGTTAGTAACTGCTAATGGAACGGTTTCGATATGGATACAC |
| | AAAAAAA |
| 296 | GenBank Accession No. KX262887 (Zika virus isolate 103451, complete genome) |
| | GTTGTTGATCTGTGTGAATCAGACTGCGACAGTTCGAGTTTGAAGCGAAAGCTAGCAACAGTATCAA |
| | CAGGTTTTATTTTGGATTTGGAAACGAGAGTTTCTGGTCATGAAAAACCCAAAAAAGAAATCCGGAG |
| | GATTCCGGATTGTCAATATGCTAAAACGCGGAGTAGCCCGTGTGAGCCCCTTTGGGGGCTTGAAGAG |
| | GCTGCCAGCCGGACTTCTGCTGGGTCATGGGCCCATCAGGATGGTCTTGGCGATTCTAGCCTTTTTG |
| | AGATTCACGGCAATCAAGCCATCACTGGGTCTCATCAATAGATGGGGTTCAGTGGGGAAAAAAGAGG |
| | CTATGGAAATAATAAAGAAGTTCAAGAAAGATCTGGCTGCCATGCTGAGAATAATCAATGCTAGGAA |
| | GGAGAAGAAGAGACGAGGCGCAGATACTAGTGTCGGAATTGTTGGCCTCCTGCTGACCACAGCTATG |
| | GCAGCGGAGGTCACTAGACGTGGGAGTGCATACTATATGTACTTGGACAGAAACGATGCTGGGGAGG |
| | CCATATCTTTTCCAACCACATTGGGGATGAATAAGTGTTATATACAGATCATGGATCTTGGACACAT |
| | GTGTGATGCCACCATGAGCTATGAATGCCCTATGCTGGATGAGGGGGTGGAACCAGATGACGTCGAT |
| | TGTTGGTGCAACACGACGTCAACTTGGGTTGTGTACGGAACCTGCCATCACAAAAAAGGTGAAGCAC |
| | GGAGATCTAGAAGAGCTGTGACGCTCCCCTCCCATTCCACTAGGAAGCTGCAAACGCGGTCGCAAAC |
| | CTGGTTGGAATCAAGAGAATACACAAAGCACTTGATTAGAGTCGAAAATTGGATATTCAGGAACCCT |
| | GGCTTCGCGTTAGCAGCAGCTGCCATCGCTTGGCTTTTGGGAAGCTCAACGAGCCAAAAAGTCATAT |
| | ACTTGGTCATGATACTGCTGATTGCCCCGGCATACAGCATCAGGTGCATAGGAGTCAGCAATAGGGA |
| | CTTTGTGGAAGGTATGTCAGGTGGGACTTGGGTTGATGTTGTCTTGGAACATGGAGGTTGTGTCACC |
| | GTAATGGCACAGGACAAACCGACTGTCGACATAGAGCTGGTTACAACAACAGTCAGCAACATGGCGG |
| | AGGTAAGATCCTACTGCTATGAGGCATCAATATCAGACATGGCTTCGGACAGCCGCTGCCCAACACA |
| | AGGTGAAGCCTACCTTGACAAGCAATCAGACACTCAATATGTCTGCAAAAGAACGTTAGTGGACAGA |
| | GGCTGGGGAAATGGATGTGGACTTTTTGGCAAAGGGAGCCTGGTGACATGCGCTAAGTTTGCATGCT |
| | CCAAGAAAATGACCGGGAAGAGCATCCAGCCAGAGAATCTGGAGTACCGGATAATGCTGTCAGTTCA |
| | TGGCTCCCAGCACAGTGGGATGATCGTTAATGACACAGGACATGAAACTGATGAGAATAGAGCGAAG |
| | GTTGAGATAACGCCCAATTCACCAAGAGCCGAAGCCACCCTGGGGGGTTTTGGAAGCCTAGGACTTG |
| | ATTGTGAACCGAGGACAGGCCTTGACTTTTCAGATTTGTATTACTTGACTATGAATAACAAGCACTG |
| | GTTGGTTCACAAGGAGTGGTTCCACGACATTCCATTACCTTGGCACGCTGGGGCAGACACCGGAACT |
| | CCACACTGGAACAACAAAGAAGCACTGGTAGAGTTCAAGGACGCACATGCCAAAAGGCAAACTGTCG |
| | TGGTTCTAGGGAGTCAAGAAGGAGCAGTTCACACGGCCCTTGCTGGAGCTCTGGAGGCTGAGATGGA |
| | TGGTGCAAAGGGAAGGCTGTCCTCTGGCCACTTGAAATGTCGCCTGAAAATGGATAAACTTAGATTG |
| | AAGGGCGTGTCATACTCCTTGTGTACCGCAGCGTTCACATTCACCAAGATCCCGGCTGAAACACTGC |
| | ACGGGACAGTCACAGTGGAGGTACAGTACGCAGGGACAGATGGACCTTGCAAGGTTCCAGCTCAGAT |
| | GGCGGTGGACATGCAAACTCTGACCCCAGTTGGGAGGTTGATAACCGCTAACCCCGTAATCACTGAA |
| | AGCACTGAGAACTCTAAGATGATGCTGGAACTTGATCCACCATTTGGGGACTCTTACATTGTCATAG |
| | GAGTCGGGGAGAAGAAGATCACCCACCACTGGCACAGGAGTGGCAGCACCATTGGAAAAGCATTTGA |
| | AGCCACTGTGAGAGGTGCCAAGAGAATGGCAGTCTTGGGAGACACAGCCTGGGACTTTGGATCAGTT |
| | GGAGGCGCTCTCAACTCATTGGGCAAGGGCATCCATCAAATTTTTGGAGCAGCTTTCAAATCATTGT |
| | TTGGAGGAATGTCCTGGTTCTCACAAATTCTCATTGGAACGTTGCTGATGTGGTTGGGTCTGAACAC |
| | AAAGAATGGATCTATTTCCCTTATGTGCTTGGCCTTAGGGGGAGTGTTGATCTTCTTATCCACAGCC |
| | GTCTCTGCTGATGTGGGGTGCTCGGTGGACTTCTCAAAGAAGGAGACGAGATGCGGTACAGGGGTGT |
| | TCGTCTATAACGACGTTGAAGCCTGGAGGGACAGGTACAAGTACCATCCTGACTCCCCCCGTAGATT |
| | GGCAGCAGCAGTCAAGCAAGCCTGGGAAGATGGTATCTGCGGGATCTCCTCTGTTTCAAGAATGGAA |
| | AACATCATGTGGAGATCAGTAGAAGGGGAGCTCAACGCAATCCTGGAAGAGAATGGAGTTCAACTGA |
| | CGGTCGTTGTGGGATCTGTAAAAAACCCCATGTGGAGAGCTCCACAGAGATTGCCCGTGCCTGTGAA |
| | CGAGCTGCCCCACGGCTGGAAGGCTTGGGGGAAATCGTACTTCGTCAGAGCAGCAAAGACAAATAAC |
| | AGCTTTGTCGTGGATGGTGACACACTGAAGGAATGCCCACTCAAACATAGAGCATGGAACAGCTTTC |
| | TTGTGGAGGATCATGGGTTCGGGGTATTTCACACTAGTGTCTGGCTCAAGGTTAGAGAAGATTATTC |
| | ATTAGAGTGTGATCCAGCCGTTATTGGAACAGCTGTTAAGGGAAAGGAGGCTGTACACAGTGATCTA |
| | GGCTACTGGATTGAGAGTGAGAAGAATGACACATGGAGGCTGAAGAGGGCCCATCTGATCGAGATGA |
| | AAACATGTGAATGGCCAAAGTCCCACACATTGTGGACAGATGGAATAGAAGAGAGTGATCTGATCAT |
| | ACCCAAGTCTTTAGCTGGGCCACTCAGCCATCACAATACCAGAGAGGGCTACAGGACCCAAATGAAA |
| | GGGCCATGGCACAGTGAAGAGCTTGAAATTCGGTTTGAGGAATGCCCAGGCACTAAGGTCCACGTGG |
| | AGGAAACATGTGGAACAAGAGGACCATCTCTGAGATCAACCACTGCAAGCGGAAGGGTGATCGAGGA |
| | ATGGTGCTGCAGGGAGTGCACAATGCCCCCACTGTCGTTCCGGGCTAAAGATGGCTGTTGGTATGGA |
| | ATGGAGATAAGGCCCAGGAAAGAACCAGAAAGCAACTTAGTAAGGTCAATGGTGACTGCAGGATCAA |
| | CTGATCACATGGATCACTTCTCCCTTGGAGTGCTTGTGATTCTGCTCATGGTGCAGGAAGGGCTAAA |
| | GAAGAGAATGACCACAAAGATCATCATAAGCACATCAATGGCAGTGCTGGTAGCTATGATCCTGGGA |
| | GGATTTTCAATGAGTGACCTGGCTAAGCTTGCAATTTTGATGGGTGCCACCTTCGCGGAAATGAACA |
| | CTGGAGGAGATGTAGCTCATCTGGCGCTGATAGCGGCATTCAAAGTCAGACCAGCGTTGCTGGTATC |
| | TTTCATCTTCAGAGCTAATTGGACACCCCGTGAAAGCATGCTACTGGCCTTGGCCTCGTGTCTTTTG |
| | CAAACTGCGATCTCCGCCTTGGAAGGCGACCTGATGGTTCTCATCAATGGTTTTGCTTTGGCCTGGT |
| | TGGCAATACGAGCGATGGTTGTTCCACGCACTGATAACATCACCTTGGCAATCCTGGCTGCTCTGAC |
| | ACCACTGGCCCGGGGCACACTGCTTGTGGCGTGGAGAGCAGGCCTTGCTACTTGCGGGGGGTTTATG |
| | CTCCTCTCTCTGAAGGGAAAAGGCAGTGTGAAGAAGAACTTACCATTTGTCATGGCCCTGGGACTAA |
| | CCGCTGTGAGGCTGGTCGACCCCATCAACGTGGTGGGACTGCTGTTGCTCACAAGGAGTGGGAAGCG |
| | GAGCTGGCCCCCTAGCGAAGTACTCACAGCTGTTGGCCTGATATGCGCATTGGCTGGAGGGTTCGCC |
| | AAGGCAGATATAGAGATGGCTGGGCCCATGGCCGCGGTCGGTCTGCTAATTGTCAGTTACGTGGTCT |
| | CAGGAAAGAGTGTGGACATGTACATTGAAAGAGCAGGTGACATCACATGGGAAAAAGATGCGGAAGT |
| | CACTGGAAACAGTCCCCGGCTCGATGTGGCGCTAGATGAGAGTGGTGATTTCTCCCTGGTGGAGGAT |
| | GACGGTCCCCCCATGAGAGAGATCATACTCAAGGTGGTCCTGATGACCATCTGTGGCATGAACCCAA |
| | TAGCCATACCCTTCGCAGCTGGAGCGTGGTACGTATACGTGAAGACTGGAAAAAGGAGTGGTGCTCT |
| | ATGGGATGTGCCTGCTCCCAAGGAAGTAAAAAAGGGGGAGACCACAGATGGAGTGTACAGAGTAATG |
| | ACTCGTAGACTGCTAGGTTCAACACAAGTTGGAGTGGGAGTTATGCAAGAGGGGGTCTTTCACACTA |
| | TGTGGCACGTCACAAAAGGATCCGCACTGAGAAGCGGTGAAGGGAGACTTGATCCATACTGGGGAGA |
| | TGTCAAGCAGGATCTGGTGTCATACTGTGGTCCATGGAAGCTAGATGCCGCCTGGGACGGGCACAGC |
| | GAGGTGCAGCTCTTGGCCGTGCCCCCCGGAGAGAGAGCGAGGAACATCCAGACTCTGCCCGGAATAT |
| | TTAAGACAAAGGATGGGGACATTGGAGCGGTTGCGCTGGATTACCCAGCAGGAACTTCAGGATCTCC |
| | AATCCTAGACAAGTGTGGGAGAGTGATAGGACTTTATGGCAATGGGGTCGTGATCAAAAATGGGAGT |
| | TATGTTAGTGCCATCACCCAAGGGAGGAGGGAGGAAGAGACTCCTGTTGAGTGCTTCGAGCCTTCGA |
| | TGCTGAAGAAGAAGCAGCTAACTGTCTTAGACTTACATCCTGGAGCTGGGAAAACCAGGAGAGTTCT |
| | TCCTGAAATAGTCCGTGAAGCCATAAAAACAAGACTCCGTACTGTGATCTTAGCTCCAACCAGGGTT |
| | GTCGCTGCTGAAATGGAGGAGGCCCTTAGAGGGCTTCCAGTGCGTTATATGACAACAGCAGTCAATG |
| | TCACCCACTCTGGAACAGAAATCGTCGACTTAATGTGCCATGCCACCTTCACTTCACGTCTACTACA |
| | GCCAATCAGAGTCCCCAACTATAATCTGTATATTATGGATGAGGCCCACTTCACAGATCCCTCAAGT |
| | ATAGCAGCAAGAGGATACATTTCAACAAGGGTTGAGATGGGCGAGGCGGCTGCCATCTTCATGACCG |
| | CCACGCCACCAGGAACCCGTGACGCATTTCCGGACTCCAACTCACCAATTATGGACACCGAAGTGGA |
| | AGTCCCAGAGAGAGCCTGGAGCTCAGGCTTTGATTGGGTGACGGATCATTCTGGAAAAACAGTTTGG |
| | TTTGTTCCAAGCGTGAGGAACGGCAATGAGATCGCAGCTTGTCTGACAAAGGCTGGAAAACGGGTCA |
| | TACAGCTCAGCAGAAAGACTTTTGAGACAGAGTTCCAGAAAACAAAACATCAAGAGTGGGACTTTGT |
| | CGTGACAACTGACATTTCAGAGATGGGCGCCAACTTTAAAGCTGACCGTGTCATAGATTCCAGGAGA |
| | TGCCTAAAGCCGGTCATACTTGATGGCGAGAGAGTCATTCTGGCTGGACCCATGCCTGTCACACATG |
| | CCAGCGCTGCCCAGAGGAGGGGGCGCATAGGCAGGAATCCCAACAAACCTGGAGATGAGTATCTGTA |
| | TGGAGGTGGGTGCGCAGAGACTGACGAAGACCATGCACACTGGCTTGAAGCAAGAATGCTCCTTGAC |
| | AATATTTACCTCCAAGATGGCCTCATAGCCTCGCTCTATCGACCTGAGGCCGACAAAGTAGCAGCCA |
| | TTGAGGGAGAGTTCAAGCTTAGGACGGAGCAAAGGAAGACCTTTGTGGAACTCATGAAAAGAGGAGA |
| | TCTTCCTGTTTGGCTGGCCTATCAGGTTGCATCTGCCGGAATAACCTACACAGATAGAAGATGGTGC |
| | TTTGATGGCACGACCAACAACACCATACTGGAAGACAGCGTGCCGGCAGAGGTGTGGACCAGACACG |
| | GAGAGAAAAGAGTGCTCAAACCGAGGTGGATGGACGCCAGAGTTTGTTCAGATCATGCGGCCCTGAA |
| | GTCATTCAAGGAGTTTGCCGCTGGGAAAAGAGGAGCGGCTTTTGGAGTGATGGAAGCCCTGGGAACA |
| | CTGCCAGGACACATGACAGAGAGATTCCAGGAAGCCATTGACAACCTCGCTGTGCTCATGCGGGCAG |
| | AGACTGGAAGCAGGCCTTACAAAGCCGCGGCGGCCCAATTGCCGGAGACCCTAGAGACCATTATGCT |
| | TTTGGGGTTGCTGGGAACAGTCTCGCTGGGAATCTTTTTCGTCTTGATGAGGAACAAGGGCATAGGG |
| | AAGATGGGCTTTGGAATGGTGACCCTTGGGGCCAGTGCATGGCTCATGTGGCTCTCGGAAATTGAGC |
| | CAGCCAGAATTGCATGCGTCCTCATTGTTGTGTTCCTATTGCTGGTGGTGCTCATACCTGAGCCAGA |
| | AAAGCAAAGATCTCCCCAGGACAACCAAATGGCAATCATCATCATGGTAGCAGTAGGTCTTCTGGGC |
| | TTGATTACCGCCAATGAACTCGGATGGTTGGAGAGAACAAAGAGTGACCTAAGCCATCTAATGGGAA |
| | GGAGAGAGGAGGGGGCAACCATAGGATTCTCAATGGACATTGACCTGCGGCCAGCCTCAGCTTGGGC |
| | CATCTATGCTGCCTTGACAACTTTCATTACCCCAGCCGTCCAACATGCAGTGACCACTTCATACAAC |
| | AACTACTCCTTAATGGCGATGGCCACGCAAGCTGGAGTGTTGTTTGGTATGGGCAAAGGGATGCCAT |
| | TCTACGCATGGGACTTTGGAGTCCCGCTGCTAATGATAGGTTGCTACTCACAATTAACACCCCTGAC |
| | CCTAATAGTGGCCATCATTTTGCTCGTGGCGCACTACATGTACTTGATCCCAGGGCTGCAGGCAGCA |
| | GCTGCGCGTGCTGCCCAGAAGAGAACGGCAGCTGGCATCATGAAGAACCCTGTTGTGGATGGAATAG |
| | TGGTGACTGACATTGACACAATGACAATTGACCCCCAAGTGGAGAAAAAGATGGGACAGGTGCTACT |
| | CATAGCAGTAGCCGTCTCCAGCGCCATACTGTCGCGGACCGCCTGGGGGTGGGGGGAGGCTGGGGCC |
| | CTGATCACAGCCGCAACTTCCACTTTGTGGGAAGGCTCTCCGAACAAGTACTGGAACTCCTCTACAG |
| | CCACTTCACTGTGTAACATTTTTAGGGGAAGTTACTTGGCTGGAGCCTCTCTAATCTACACAGTAAC |
| | AAGAAACGCTGGCTTGGTCAAGAGACGTGGGGGTGGAACAGGAGAGACCCTGGGAGAGAAATGGAAG |
| | GCCCGCTTGAACCAGATGTCGGCCCTGGAGTTCTACTCCTACAAAAAGTCAGGCATCACCGAGGTGT |
| | GCAGAGAAGAGGCCCGCCGCGCCCTCAAGGACGGTGTGGCAACGGGAGGCCATGCTGTGTCCCGAGG |
| | AAGTGCAAAGCTGAGATGGTTGGTGGAGCGGGGATACCTGCAGCCCTATGGAAAGGTCATTGATCTT |
| | GGATGTGGCAGAGGGGGCTGGAGTTACTACGCCGCCACCATCCGCAAAGTTCAAGAAGTGAAAGGAT |
| | ACACAAAAGGAGGCCCTGGTCATGAAGAACCCGTGTTGGTGCAAAGCTATGGGTGGAACATTGTCCG |
| | TCTTAAGAGTGGGGTGGACGTCTTTCATATGGCGGCTGAGCCGTGTGACACGTTGCTGTGTGACATA |
| | GGTGAGTCATCATCTAGTCCTGAAGTGGAAGAAGCACGGACGCTCAGAGTCCTCTCCATGGTGGGGG |
| | ATTGGCTTGAAAAAAGACCAGGAGCCTTTTGTATAAAAGTGTTGTGCCCATACACCAGCACTATGAT |
| | GGAAACCCTGGAGCGACTGCAGCGTAGGTATGGGGGAGGACTGGTCAGAGTGCCACTCTCCCGCAAC |
| | TCTACACATGAGATGTACTGGGTCTCTGGAGCGAAAAGCAACACCATAAAAAGTGTGTCCACCACGA |
| | GCCAGCTCCTCTTGGGGCGCATGGACGGGCCTAGGAGGCCAGTGAAATATGAGGAGGATGTGAATCT |
| | CGGCTCTGGCACGCGGGCTGTGGTAAGCTGCGCTGAAGCTCCCAACATGAAGATCATTGGTAACCGC |
| | ATTGAAAGGATCCGCAGTGAGCACGCGGAAACGTGGTTCTTTGACGAGAACCACCCATATAGGACAT |
| | GGGCTTACCATGGAAGCTATGAGGCCCCCACACAAGGGTCAGCGTCCTCTCTAATAAACGGGGTTGT |
| | CAGGCTCCTGTCAAAACCCTGGGATGTGGTGACTGGAGTCACAGGAATAGCCATGACCGACACCACA |
| | CCGTATGGTCAGCAAAGAGTTTTCAAGGAAAAAGTGGACACTAGGGTGCCAGACCCCCAAGAAGGCA |
| | CTCGTCAGGTTATGAGCATGGTCTCTTCCTGGTTGTGGAAAGAGCTAGGCAAACACAAACGGCCACG |
| | AGTCTGTACCAAAGAAGAGTTCATCAACAAGGTTCGTAGCAATGCAGCATTAGGGGCAATATTTGAA |
| | GAGGAAAAAGAGTGGAAGACTGCAGTGGAAGCTGTGAAGGATCGAAGGTTCTGGGCTCTAGTGGACA |
| | AGGAAAGAGAGCACCACCTGAGAGGAGAGTGCCAGAGTTGTGTGTACAACATGATGGGAAAAAGAGA |
| | AAAGAAACAAGGGGAATTTGGAAAGGCCAAGGGCAGCCGCGCCATCTGGTATATGTGGCTAGGGGCT |
| | AGATTTCTAGAGTTCGAAGCCCTTGGATTCTTGAACGAGGATCACTGGATGGGGAGAGAGAACTCAG |
| | GAGGTGGTGTTGAAGGGCTGGGATTACAAAGACTCGGATATGTCCTAGAAGAGATGAGTTGCATACC |
| | AGGAGGAAGGATGTATGCAGATGACACTGCTGGCTGGGACACCCGCATCAGCAGGTTTGATCTGGAG |
| | AATGAAGCTCTAATCACCAACCAAATGGAGAAAGGGCACAGGGCCTTGGCATTGGCCATAATCAAGT |
| | ACACATACCAAAACAAAGTGGTAAAGGTCCTTAGACCAGCTGAAAAAGGGAAAACAGTTATGGACAT |
| | TATTTCGAGACAAGACCAAAGGGGGAGCGGACAAGTTGTCACTTACGCTCTTAACACATTTACCAAC |
| | CTAGTGGTGCAACTCATTCGGAATATGGAGGCTGAGGAAGTTCTAGAGATGCAAGACTTGTGGCTGC |
| | TGCGGAGGTCAGAGAAAGTGACCAACTGGTTGCAGAGCAACGGATGGGATAGGCTCAAACGAATGGC |
| | AGTCAGTGGAGATGATTGCGTTGTGAAGCCAATTGATGATAGGTTTGCACATGCCCTCAGGTTCTTG |
| | AATGATATGGGAAAAGTTAGGAAGGACACACAAGAGTGGAAACCCTCAACTGGATGGGACAACTGGG |
| | AAGAAGTTCCGTTTTGCTCCCACCACTTCAACAAGCTCCATCTCAAGGACGGGAGGTCCATTGTGGT |
| | TCCCTGCCGCCACCAAGATGAACTGATTGGCCGGGCCCGCGTCTCTCCAGGGGCGGGATGGAGCATC |
| | CGGGAGACTGCTTGCCTAGCAAAATCATATGCGCAAATGTGGCAGCTCCTTTATTTCCACAGAAGGG |
| | ACCTCCGACTGATGGCCAATGCCATTTGTTCATCTGTGCCAGTTGACTGGGTTCCAACTGGGAGAAC |
| | TACCTGGTCAATCCATGGAAAGGGAGAATGGATGACCACTGAAGACATGGTTGTGGTGTGGAACAGA |
| | GTGTGGATTGAGGAGAACGACCACATGGAAGACAAGACCCCAGTTACGAAATGGACAGACATTCCCT |
| | ATTTGGGAAAAAGGGAAGACTTGTGGTGTGGATCTCTCATAGGGCACAGACCGCGCACCACCTGGGC |
| | TGAGAACATTAAAAACACAGTCAACATGGTGCGCAGGATCATAGGTGATGAAGAAAAGTACATGGAC |
| | TACCTATCCACCCAAGTTCGCTACTTGGGTGAAGAAGGGTCTACACCTGGAGTGCTGTAAGCACCAA |
| | TCTTAACGTTGTCAGGCCTGCTAGTCAGCCACAGCTTGGGGAAAGCTGTGCAGCCTGTGACCCCCCC |
| | AGGAGAAGCTGGGAAACCAAGCCTATAGTCAGGCCGAGAACGCCATGGCACGGAAGAAGCCATGCTG |
| | CCTGTGAGCCCCTCAGAGGACACTGAGTCAAAAAACCCCACGCGCTTGGAGGCGCAGGATGGGAAAA |
| | GAAGGTGGCGACCTTCCCCACCCTTCAATCTGGGGCCTGAACTGGAGATCAGCTGTGGATCTCCAGA |
| | AGAGGGACTAGTGGTTAGAGGAGACCCCCCGGAAAACGCAAAACAGCATATTGACGCTGGGAAAGAC |
| | CAGAGACTCCATGAGTTTCCACCACGCTGGCCGCCAGGCACAGATCGCCGAATAGCGGCGGCCGGTG |
| | TGGGGAAATCCATGGGTCT |
| 297 | GenBank Accession No. AY632535.2 (Zika virus strain MR 766, complete |
| | genome) |
| | AGTTGTTGATCTGTGTGAGTCAGACTGCGACAGTTCGAGTCTGAAGCGAGAGCTAACAACAGTATCA |
| | ACAGGTTTAATTTGGATTTGGAAACGAGAGTTTCTGGTCATGAAAAACCCCAAAGAAGAAATCCGGA |
| | GGATCCGGATTGTCAATATGCTAAAACGCGGAGTAGCCCGTGTAAACCCCTTGGGAGGTTTGAAGAG |
| | GTTGCCAGCCGGACTTCTGCTGGGTCATGGACCCATCAGAATGGTTTTGGCGATACTAGCCTTTTTG |
| | AGATTTACAGCAATCAAGCCATCACTGGGCCTTATCAACAGATGGGGTTCCGTGGGGAAAAAAGAGG |
| | CTATGGAAATAATAAAGAAGTTCAAGAAAGATCTTGCTGCCATGTTGAGAATAATCAATGCTAGGAA |
| | AGAGAGGAAGAGACGTGGCGCAGACACCAGCATCGGAATCATTGGCCTCCTGCTGACTACAGCCATG |
| | GCAGCAGAGATCACTAGACGCGGGAGTGCATACTACATGTACTTGGATAGGAGCGATGCCGGGAAGG |
| | CCATTTCGTTTGCTACCACATTGGGAGTGAACAAGTGCCACGTACAGATCATGGACCTCGGGCACAT |
| | GTGTGACGCCACCATGAGTTATGAGTGCCCTATGCTGGATGAGGGAGTGGAACCAGATGATGTCGAT |
| | TGCTGGTGCAACACGACATCAACTTGGGTTGTGTACGGAACCTGTCATCACAAAAAAGGTGAGGCAC |
| | GGCGATCTAGAAGAGCCGTGACGCTCCCTTCTCACTCTACAAGGAAGTTGCAAACGCGGTCGCAGAC |
| | CTGGTTAGAATCAAGAGAATACACGAAGCACTTGATCAAGGTTGAAAACTGGATATTCAGGAACCCC |
| | GGGTTTGCGCTAGTGGCCGTTGCCATTGCCTGGCTTTTGGGAAGCTCGACGAGCCAAAAAGTCATAT |
| | ACTTGGTCATGATACTGCTGATTGCCCCGGCATACAGTATCAGGTGCATTGGAGTCAGCAATAGAGA |
| | CTTCGTGGAGGGCATGTCAGGTGGGACCTGGGTTGATGTTGTCTTGGAACATGGAGGCTGCGTTACC |
| | GTGATGGCACAGGACAAGCCAACAGTCGACATAGAGTTGGTCACGACGACGGTTAGTAACATGGCCG |
| | AGGTAAGATCCTATTGCTACGAGGCATCGATATCGGACATGGCTTCGGACAGTCGTTGCCCAACACA |
| | AGGTGAAGCCTACCTTGACAAGCAATCAGACACTCAATATGTCTGCAAAAGAACATTAGTGGACAGA |
| | GGTTGGGGAAACGGTTGTGGACTTTTTGGCAAAGGGAGCTTGGTGACATGTGCCAAGTTTACGTGTT |
| | CTAAGAAGATGACCGGGAAGAGCATTCAACCGGAAAATCTGGAGTATCGGATAATGCTATCAGTGCA |
| | TGGCTCCCAGCATAGCGGGATGATTGGATATGAAACTGACGAAGATAGAGCGAAAGTCGAGGTTACG |
| | CCTAATTCACCAAGAGCGGAAGCAACCTTGGGAGGCTTTGGAAGCTTAGGACTTGACTGTGAACCAA |
| | GGACAGGCCTTGACTTTTCAGATCTGTATTACCTGACCATGAACAATAAGCATTGGTTGGTGCACAA |
| | AGAGTGGTTTCATGACATCCCATTGCCTTGGCATGCTGGGGCAGACACCGGAACTCCACACTGGAAC |
| | AACAAAGAGGCATTGGTAGAATTCAAGGATGCCCACGCCAAGAGGCAAACCGTCGTCGTTCTGGGGA |
| | GCCAGGAAGGAGCCGTTCACACGGCTCTCGCTGGAGCTCTAGAGGCTGAGATGGATGGTGCAAAGGG |
| | AAGGCTGTTCTCTGGCCATTTGAAATGCCGCCTAAAAATGGACAAGCTTAGATTGAAGGGCGTGTCA |
| | TATTCCTTGTGCACTGCGGCATTCACATTCACCAAGGTCCCAGCTGAAACACTGCATGGAACAGTCA |
| | CAGTGGAGGTGCAGTATGCAGGGACAGATGGACCCTGCAAGATCCCAGTCCAGATGGCGGTGGACAT |
| | GCAGACCCTGACCCCAGTTGGAAGGCTGATAACCGCCAACCCCGTGATTACTGAAAGCACTGAGAAC |
| | TCAAAGATGATGTTGGAGCTTGACCCACCATTTGGGGATTCTTACATTGTCATAGGAGTTGGGGACA |
| | AGAAAATCACCCACCACTGGCATAGGAGTGGTAGCACCATCGGAAAGGCATTTGAGGCCACTGTGAG |
| | AGGCGCCAAGAGAATGGCAGTCCTGGGGGATACAGCCTGGGACTTCGGATCAGTCGGGGGTGTGTTC |
| | AACTCACTGGGTAAGGGCATTCACCAGATTTTTGGAGCAGCCTTCAAATCACTGTTTGGAGGAATGT |
| | CCTGGTTCTCACAGATCCTCATAGGCACGCTGCTAGTGTGGTTAGGTTTGAACACAAAGAATGGATC |
| | TATCTCCCTCACATGCTTGGCCCTGGGGGGAGTGATGATCTTCCTCTCCACGGCTGTTTCTGCTGAC |
| | GTGGGGTGCTCAGTGGACTTCTCAAAAAAGGAAACGAGATGTGGCACGGGGGTATTCATCTATAATG |
| | ATGTTGAAGCCTGGAGGGACCGGTACAAGTACCATCCTGACTCCCCCCGCAGATTGGCAGCAGCAGT |
| | CAAGCAGGCCTGGGAAGAGGGGATCTGTGGGATCTCATCCGTTTCAAGAATGGAAAACATCATGTGG |
| | AAATCAGTAGAAGGGGAGCTCAATGCTATCCTAGAGGAGAATGGAGTTCAACTGACAGTTGTTGTGG |
| | GATCTGTAAAAAACCCCATGTGGAGAGGTCCACAAAGATTGCCAGTGCCTGTGAATGAGCTGCCCCA |
| | TGGCTGGAAAGCCTGGGGGAAATCGTATTTTGTTAGGGCGGCAAAGACCAACAACAGTTTTGTTGTC |
| | GACGGTGACACACTGAAGGAATGTCCGCTTGAGCACAGAGCATGGAATAGTTTTCTTGTGGAGGATC |
| | ACGGGTTTGGAGTCTTCCACACCAGTGTCTGGCTTAAGGTCAGAGAAGATTACTCATTAGAATGTGA |
| | CCCAGCCGTCATAGGAACAGCTGTTAAGGGAAGGGAGGCCGCGCACAGTGATCTGGGCTATTGGATT |
| | GAAAGTGAAAAGAATGACACATGGAGGCTGAAGAGGGCCCACCTGATTGAGATGAAAACATGTGAAT |
| | GGCCAAAGTCTCACACATTGTGGACAGATGGAGTAGAAGAAAGTGATCTTATCATACCCAAGTCTTT |
| | AGCTGGTCCACTCAGCCACCACAACACCAGAGAGGGTTACAGAACCCAAGTGAAAGGGCCATGGCAC |
| | AGTGAAGAGCTTGAAATCCGGTTTGAGGAATGTCCAGGCACCAAGGTTTACGTGGAGGAGACATGCG |
| | GAACTAGAGGACCATCTCTGAGATCAACTACTGCAAGTGGAAGGGTCATTGAGGAATGGTGCTGTAG |
| | GGAATGCACAATGCCCCCACTATCGTTTCGAGCAAAAGACGGCTGCTGGTATGGAATGGAGATAAGG |
| | CCCAGGAAAGAACCAGAGAGCAACTTAGTGAGGTCAATGGTGACAGCGGGGTCAACCGATCATATGG |
| | ACCACTTCTCTCTTGGAGTGCTTGTGATTCTACTCATGGTGCAGGAGGGGTTGAAGAAGAGAATGAC |
| | CACAAAGATCATCATGAGCACATCAATGGCAGTGCTGGTAGTCATGATCTTGGGAGGATTTTCAATG |
| | AGTGACCTGGCCAAGCTTGTGATCCTGATGGGTGCTACTTTCGCAGAAATGAACACTGGAGGAGATG |
| | TAGCTCACTTGGCATTGGTAGCGGCATTTAAAGTCAGACCAGCCTTGCTGGTCTCCTTCATTTTCAG |
| | AGCCAATTGGACACCCCGTGAGAGCATGCTGCTAGCCCTGGCTTCGTGTCTTCTGCAAACTGCGATC |
| | TCTGCTCTTGAAGGTGACTTGATGGTCCTCATTAATGGATTTGCTTTGGCCTGGTTGGCAATTCGAG |
| | CAATGGCCGTGCCACGCACTGACAACATCGCTCTACCAATCTTGGCTGCTCTAACACCACTAGCTCG |
| | AGGCACACTGCTCGTGGCATGGAGAGCGGGCCTGGCTACTTGTGGAGGGATCATGCTCCTCTCCCTG |
| | AAAGGGAAAGGTAGTGTGAAGAAGAACCTGCCATTTGTCATGGCCCTGGGATTGACAGCTGTGAGGG |
| | TAGTAGACCCTATTAATGTGGTAGGACTACTGTTACTCACAAGGAGTGGGAAGCGGAGCTGGCCCCC |
| | TAGTGAAGTTCTCACAGCCGTTGGCCTGATATGTGCACTGGCCGGAGGGTTTGCCAAGGCAGACATT |
| | GAGATGGCTGGACCCATGGCTGCAGTAGGCTTGCTAATTGTCAGCTATGTGGTCTCGGGAAAGAGTG |
| | TGGACATGTACATTGAAAGAGCAGGTGACATCACATGGGAAAAGGACGCGGAAGTCACTGGAAACAG |
| | TCCTCGGCTTGACGTGGCACTGGATGAGAGTGGTGACTTCTCCTTGGTAGAGGAAGATGGTCCACCC |
| | ATGAGAGAGATCATACTCAAGGTGGTCCTGATGGCCATCTGTGGCATGAACCCAATAGCTATACCTT |
| | TTGCTGCAGGAGCGTGGTATGTGTATGTGAAGACTGGGAAAAGGAGTGGCGCCCTCTGGGACGTGCC |
| | TGCTCCCAAAGAAGTGAAGAAAGGAGAGACCACAGATGGAGTGTACAGAGTGATGACTCGCAGACTG |
| | CTAGGTTCAACACAGGTTGGAGTGGGAGTCATGCAAGAGGGAGTCTTCCACACCATGTGGCACGTTA |
| | CAAAAGGAGCCGCACTGAGGAGCGGTGAGGGAAGACTTGATCCATACTGGGGGGATGTCAAGCAGGA |
| | CTTGGTGTCATACTGTGGGCCTTGGAAGTTGGATGCAGCTTGGGATGGACTCAGCGAGGTACAGCTT |
| | TTGGCCGTACCTCCCGGAGAGAGGGCCAGAAACATTCAGACCCTGCCTGGAATATTCAAGACAAAGG |
| | ACGGGGACATCGGAGCAGTTGCTCTGGACTACCCTGCAGGGACCTCAGGATCTCCGATCCTAGACAA |
| | ATGTGGAAGAGTGATAGGACTCTATGGCAATGGGGTTGTGATCAAGAATGGAAGCTATGTTAGTGCT |
| | ATAACCCAGGGAAAGAGGGAGGAGGAGACTCCGGTTGAATGTTTCGAACCCTCGATGCTGAAGAAGA |
| | AGCAGCTAACTGTCTTGGATCTGCATCCAGGAGCCGGAAAAACCAGGAGAGTTCTTCCTGAAATAGT |
| | CCGTGAAGCCATAAAAAAGAGACTCCGGACAGTGATCTTGGCACCAACTAGGGTTGTCGCTGCTGAG |
| | ATGGAGGAGGCCTTGAGAGGACTTCCGGTGCGTTACATGACAACAGCAGTCAACGTCACCCATTCTG |
| | GGACAGAAATCGTTGATTTGATGTGCCATGCCACTTTCACTTCACGCTTACTACAACCCATCAGAGT |
| | CCCTAATTACAATCTCAACATCATGGATGAAGCCCACTTCACAGACCCCTCAAGTATAGCTGCAAGA |
| | GGATACATATCAACAAGGGTTGAAATGGGCGAGGCGGCTGCCATTTTTATGACTGCCACACCACCAG |
| | GAACCCGTGATGCGTTTCCTGACTCTAACTCACCAATCATGGACACAGAAGTGGAAGTCCCAGAGAG |
| | AGCCTGGAGCTCAGGCTTTGATTGGGTGACAGACCATTCTGGGAAAACAGTTTGGTTCGTTCCAAGC |
| | GTGAGAAACGGAAATGAAATCGCAGCCTGTCTGACAAAGGCTGGAAAGCGGGTCATACAGCTCAGCA |
| | GGAAGACTTTTGAGACAGAATTTCAGAAAACAAAAAATCAAGAGTGGGACTTTGTCATAACAACTGA |
| | CATCTCAGAGATGGGCGCCAACTTCAAGGCTGACCGGGTCATAGACTCTAGGAGATGCCTAAAACCA |
| | GTCATACTTGATGGTGAGAGAGTCATCTTGGCTGGGCCCATGCCTGTCACGCATGCTAGTGCTGCTC |
| | AGAGGAGAGGACGTATAGGCAGGAACCCTAACAAACCTGGAGATGAGTACATGTATGGAGGTGGGTG |
| | TGCAGAGACTGATGAAGGCCATGCACACTGGCTTGAAGCAAGAATGCTTCTTGACAACATCTACCTC |
| | CAGGATGGCCTCATAGCCTCGCTCTATCGGCCTGAGGCCGATAAGGTAGCCGCCATTGAGGGAGAGT |
| | TTAAGCTGAGGACAGAGCAAAGGAAGACCTTCGTGGAACTCATGAAGAGAGGAGACCTTCCCGTCTG |
| | GCTAGCCTATCAGGTTGCATCTGCCGGAATAACTTACACAGACAGAAGATGGTGCTTTGATGGCACA |
| | ACCAACAACACCATAATGGAAGACAGTGTACCAGCAGAGGTTTGGACAAAGTATGGAGAGAAGAGAG |
| | TGCTCAAACCGAGATGGATGGATGCTAGGGTCTGTTCAGACCATGCGGCCCTGAAGTCGTTCAAAGA |
| | ATTCGCCGCTGGAAAAAGAGGAGCGGCTTTGGGAGTAATGGAGGCCCTGGGAACACTGCCAGGACAC |
| | ATGACAGAGAGGTTTCAGGAAGCCATTGACAACCTCGCCGTGCTCATGCGAGCAGAGACTGGAAGCA |
| | GGCCTTATAAGGCAGCGGCAGCCCAACTGCCGGAGACCCTAGAGACCATTATGCTCTTAGGTTTGCT |
| | GGGAACAGTTTCACTGGGGATCTTCTTCGTCTTGATGCGGAATAAGGGCATCGGGAAGATGGGCTTT |
| | GGAATGGTAACCCTTGGGGCCAGTGCATGGCTCATGTGGCTTTCGGAAATTGAACCAGCCAGAATTG |
| | CATGTGTCCTCATTGTTGTGTTTTTATTACTGGTGGTGCTCATACCCGAGCCAGAGAAGCAAAGATC |
| | TCCCCAAGATAACCAGATGGCAATTATCATCATGGTGGCAGTGGGCCTTCTAGGTTTGATAACTGCA |
| | AACGAACTTGGATGGCTGGAAAGAACAAAAAATGACATAGCTCATCTAATGGGAAGGAGAGAAGAAG |
| | GAGCAACCATGGGATTCTCAATGGACATTGATCTGCGGCCAGCCTCCGCCTGGGCTATCTATGCCGC |
| | ATTGACAACTCTCATCACCCCAGCTGTCCAACATGCGGTAACCACTTCATACAACAACTACTCCTTA |
| | ATGGCGATGGCCACACAAGCTGGAGTGCTGTTTGGCATGGGCAAAGGGATGCCATTTATGCATGGGG |
| | ACCTTGGAGTCCCGCTGCTAATGATGGGTTGCTATTCACAATTAACACCCCTGACTCTGATAGTAGC |
| | TATCATTCTGCTTGTGGCGCACTACATGTACTTGATCCCAGGCCTACAAGCGGCAGCAGCGCGTGCT |
| | GCCCAGAAAAGGACAGCAGCTGGCATCATGAAGAATCCCGTTGTGGATGGAATAGTGGTAACTGACA |
| | TTGACACAATGACAATAGACCCCCAGGTGGAGAAGAAGATGGGACAAGTGTTACTCATAGCAGTAGC |
| | CATCTCCAGTGCTGTGCTGCTGCGGACCGCCTGGGGATGGGGGGAGGCTGGAGCTCTGATCACAGCA |
| | GCGACCTCCACCTTGTGGGAAGGCTCTCCAAACAAATACTGGAACTCCTCTACAGCCACCTCACTGT |
| | GCAACATCTTCAGAGGAAGCTATCTGGCAGGAGCTTCCCTTATCTATACAGTGACGAGAAACGCTGG |
| | CCTGGTTAAGAGACGTGGAGGTGGGACGGGAGAGACTCTGGGAGAGAAGTGGAAAGCTCGTCTGAAT |
| | CAGATGTCGGCCCTGGAGTTCTACTCTTATAAAAAGTCAGGTATCACTGAAGTGTGTAGAGAGGAGG |
| | CTCGCCGTGCCCTCAAGGATGGAGTGGCCACAGGAGGACATGCCGTATCCCGGGGAAGTGCAAAGAT |
| | CAGATGGTTGGAGGAGAGAGGATATCTGCAGCCCTATGGGAAGGTTGTTGACCTCGGATGTGGCAGA |
| | GGGGGCTGGAGCTATTATGCCGCCACCATCCGCAAAGTGCAGGAGGTGAGAGGATACACAAAGGGAG |
| | GTCCCGGTCATGAAGAACCCATGCTGGTGCAAAGCTATGGGTGGAACATAGTTCGTCTCAAGAGTGG |
| | AGTGGACGTCTTCCACATGGCGGCTGAGCCGTGTGACACTCTGCTGTGTGACATAGGTGAGTCATCA |
| | TCTAGTCCTGAAGTGGAAGAGACACGAACACTCAGAGTGCTCTCTATGGTGGGGGACTGGCTTGAAA |
| | AAAGACCAGGGGCCTTCTGTATAAAGGTGCTGTGCCCATACACCAGCACTATGATGGAAACCATGGA |
| | GCGACTGCAACGTAGGCATGGGGGAGGATTAGTCAGAGTGCCATTGTGTCGCAACTCCACACATGAG |
| | ATGTACTGGGTCTCTGGGGCAAAGAGCAACATCATAAAAAGTGTGTCCACCACAAGTCAGCTCCTCC |
| | TGGGACGCATGGATGGCCCCAGGAGGCCAGTGAAATATGAGGAGGATGTGAACCTCGGCTCGGGTAC |
| | ACGAGCTGTGGCAAGCTGTGCTGAGGCTCCTAACATGAAAATCATCGGCAGGCGCATTGAGAGAATC |
| | CGCAATGAACATGCAGAAACATGGTTTCTTGATGAAAACCACCCATACAGGACATGGGCCTACCATG |
| | GGAGCTACGAAGCCCCCACGCAAGGATCAGCGTCTTCCCTCGTGAACGGGGTTGTTAGACTCCTGTC |
| | AAAGCCTTGGGACGTGGTGACTGGAGTTACAGGAATAGCCATGACTGACACCACACCATACGGCCAA |
| | CAAAGAGTCTTCAAAGAAAAAGTGGACACCAGGGTGCCAGATCCCCAAGAAGGCACTCGCCAGGTAA |
| | TGAACATAGTCTCTTCCTGGCTGTGGAAGGAGCTGGGGAAACGCAAGCGGCCACGCGTCTGCACCAA |
| | AGAAGAGTTTATCAACAAGGTGCGCAGCAATGCAGCACTGGGAGCAATATTTGAAGAGGAAAAAGAA |
| | TGGAAGACGGCTGTGGAAGCTGTGAATGATCCAAGGTTTTGGGCCCTAGTGGATAGGGAGAGAGAAC |
| | ACCACCTGAGAGGAGAGTGTCACAGCTGTGTGTACAACATGATGGGAAAAAGAGAAAAGAAGCAAGG |
| | AGAGTTCGGGAAAGCAAAAGGTAGCCGCGCCATCTGGTACATGTGGTTGGGAGCCAGATTCTTGGAG |
| | TTTGAAGCCCTTGGATTCTTGAACGAGGACCATTGGATGGGAAGAGAAAACTCAGGAGGTGGAGTCG |
| | AAGGGTTAGGATTGCAAAGACTTGGATACATTCTAGAAGAAATGAATCGGGCACCAGGAGGAAAGAT |
| | GTACGCAGATGACACTGCTGGCTGGGACACCCGCATTAGTAAGTTTGATCTGGAGAATGAAGCTCTG |
| | ATTACCAACCAAATGGAGGAAGGGCACAGAACTCTGGCGTTGGCCGTGATTAAATACACATACCAAA |
| | ACAAAGTGGTGAAGGTTCTCAGACCAGCTGAAGGAGGAAAAACAGTTATGGACATCATTTCAAGACA |
| | AGACCAGAGAGGGAGTGGACAAGTTGTCACTTATGCTCTCAACACATTCACCAACTTGGTGGTGCAG |
| | CTTATCCGGAACATGGAAGCTGAGGAAGTGTTAGAGATGCAAGACTTATGGTTGTTGAGGAAGCCAG |
| | AGAAAGTGACCAGATGGTTGCAGAGCAATGGATGGGATAGACTCAAACGAATGGCGGTCAGTGGAGA |
| | TGACTGCGTTGTGAAGCCAATCGATGATAGGTTTGCACATGCCCTCAGGTTCTTGAATGACATGGGA |
| | AAAGTTAGGAAAGACACACAGGAGTGGAAACCCTCGACTGGATGGAGCAATTGGGAAGAAGTCCCGT |
| | TCTGCTCCCACCACTTCAACAAGCTGTACCTCAAGGATGGGAGATCCATTGTGGTCCCTTGCCGCCA |
| | CCAAGATGAACTGATTGGCCGAGCTCGCGTCTCACCAGGGGCAGGATGGAGCATCCGGGAGACTGCC |
| | TGTCTTGCAAAATCATATGCGCAGATGTGGCAGCTCCTTTATTTCCACAGAAGAGACCTTCGACTGA |
| | TGGCTAATGCCATTTGCTCGGCTGTGCCAGTTGACTGGGTACCAACTGGGAGAACCACCTGGTCAAT |
| | CCATGGAAAGGGAGAATGGATGAGCAGTGAGGACATGOTCATGGTGTGGAATAGAGTGTGGATTGAG |
| | GAGAACGACCATATGGAGGACAAGACTCCTGTAACAAAATGGACAGACATTCCCTATCTAGGAAAAA |
| | GGGAGGACTTATGGTGTGGATCCCTTATAGGGCACAGACCCCGCACCACTTGGGCTGAAAACATCAA |
| | AGACACAGTCAACATGGTGCGCAGGATCATAGGTGATGAAGAAAAGTACATGGACTATCTATCCACC |
| | CAAGTCCGCTACTTGGGTGAGGAAGGGTCCACACCCGGAGTGTTGTAAGCACCAATTTTAGTGTTGT |
| | CAGGCCTGCTAGTCAGCCACAGTTTGGGGAAAGCTGTGCAGCCTGTAACCCCCCCAGGAGAAGCTGG |
| | GAAACCAAGCTCATAGTCAGGCCGAGAACGCCATGGCACGGAAGAAGCCATGCTGCCTGTGAGCCCC |
| | TCAGAGGACACTGAGTCAAAAAACCCCACGCGCTTGGAAGCGCAGGATGGGAAAAGAAGGTGGCGAC |
| | CTTCCCCACCCTTCAATCTGGGGCCTGAACTGGAGACTAGCTGTGAATCTCCAGCAGAGGGACTAGT |
| | GGTTAGAGGAGACCCCCCGGAAAACGCAAAACAGCATATTGACGTGGGAAAGACCAGAGACTCCATG |
| | AGTTTCCACCACGCTGGCCGCCAGGCACAGATCGCCGAACTTCGGCGGCCGGTGTGGGGAAATCCAT |
| | GGTTTCT |
| 298 | GenBank Accession No. KJ776791.2 (Zika virus strain H/PF/2013, complete |
| | genome) |
| | AGTTGTTGATCTGTGTGAATCAGACTGCGACAGTTCGAGTTTGAAGCGAAAGCTAGCAACAGTATCA |
| | ACAGGTTTTATTTTGGATTTGGAAACGAGAGTTTCTGGTCATGAAAAACCCAAAAAAGAAATCCGGA |
| | GGATTCCGGATTGTCAATATGCTAAAACGCGGAGTAGCCCGTGTGAGCCCCTTTGGGGGCTTGAAGA |
| | GGCTGCCAGCCGGACTTCTGCTGGGTCATGGGCCCATCAGGATGGTCTTGGCGATTCTAGCCTTTTT |
| | GAGATTCACGGCAATCAAGCCATCACTGGGTCTCATCAATAGATGGGGTTCAGTGGGGAAAAAAGAG |
| | GCTATGGAAATAATAAAGAAGTTCAAGAAAGATCTGGCTGCCATGCTGAGAATAATCAATGCTAGGA |
| | AGGAGAAGAAGAGACGAGGCGCAGATACTAGTGTCGGAATTGTTGGCCTCCTGCTGACCACAGCTAT |
| | GGCAGCGGAGGTCACTAGACGTGGGAGTGCATACTATATGTACTTGGACAGAAACGACGCTGGGGAG |
| | GCCATATCTTTTCCAACCACATTGGGGATGAATAAGTGTTATATACAGATCATGGATCTTGGACACA |
| | TGTGTGATGCCACCATGAGCTATGAATGCCCTATGCTGGATGAGGGGGTGGAACCAGATGACGTCGA |
| | TTGTTGGTGCAACACGACGTCAACTTGGGTTGTGTACGGAACCTGCCATCACAAAAAAGGTGAAGCA |
| | CGGAGATCTAGAAGAGCTGTGACGCTCCCCTCCCATTCCACTAGGAAGCTGCAAACGCGGTCGCAAA |
| | CCTGGTTGGAATCAAGAGAATACACAAAGCACTTGATTAGAGTCGAAAATTGGATATTCAGGAACCC |
| | TGGCTTCGCGTTAGCAGCAGCTGCCATCGCTTGGCTTTTGGGAAGCTCAACGAGCCAAAAAGTCATA |
| | TACTTGGTCATGATACTGCTGATTGCCCCGGCATACAGCATCAGGTGCATAGGAGTCAGCAATAGGG |
| | ACTTTGTGGAAGGTATGTCAGGTGGGACTTGGGTTGATGTTGTCTTGGAACATGGAGGTTGTGTCAC |
| | CGTAATGGCACAGGACAAACCGACTGTCGACATAGAGCTGGTTACAACAACAGTCAGCAACATGGCG |
| | GAGGTAAGATCCTACTGCTATGAGGCATCAATATCGGACATGGCTTCGGACAGCCGCTGCCCAACAC |
| | AAGGTGAAGCCTACCTTGACAAGCAATCAGACACTCAATATGTCTGCAAAAGAACGTTAGTGGACAG |
| | AGGCTGGGGAAATGGATGTGGACTTTTTGGCAAAGGGAGCCTGGTGACATGCGCTAAGTTTGCATGC |
| | TCCAAGAAAATGACCGGGAAGAGCATCCAGCCAGAGAATCTGGAGTACCGGATAATGCTGTCAGTTC |
| | ATGGCTCCCAGCACAGTGGGATGATCGTTAATGACACAGGACATGAAACTGATGAGAATAGAGCGAA |
| | GGTTGAGATAACGCCCAATTCACCAAGAGCCGAAGCCACCCTGGGGGGTTTTGGAAGCCTAGGACTT |
| | GATTGTGAACCGAGGACAGGCCTTGACTTTTCAGATTTGTATTACTTGACTATGAATAACAAGCACT |
| | GGTTGGTTCACAAGGAGTGGTTCCACGACATTCCATTACCTTGGCACGCTGGGGCAGACACCGGAAC |
| | TCCACACTGGAACAACAAAGAAGCACTGGTAGAGTTCAAGGACGCACATGCCAAAAGGCAAACTGTC |
| | GTGGTTCTAGGGAGTCAAGAAGGAGCAGTTCACACGGCCCTTGCTGGAGCTCTGGAGGCTGAGATGG |
| | ATGGTGCAAAGGGAAGGCTGTCCTCTGGCCACTTGAAATGTCGCCTGAAAATGGATAAACTTAGATT |
| | GAAGGGCGTGTCATACTCCTTGTGTACCGCAGCGTTCACATTCACCAAGATCCCGGCTGAAACACTG |
| | CACGGGACAGTCACAGTGGAGGTACAGTACGCAGGGACAGATGGACCTTGCAAGGTTCCAGCTCAGA |
| | TGGCGGTGGACATGCAAACTCTGACCCCAGTTGGGAGGTTGATAACCGCTAACCCCGTAATCACTGA |
| | AAGCACTGAGAACTCTAAGATGATGCTGGAACTTGATCCACCATTTGGGGACTCTTACATTGTCATA |
| | GGAGTCGGGGAGAAGAAGATCACCCACCACTGGCACAGGAGTGGCAGCACCATTGGAAAAGCATTTG |
| | AAGCCACTGTGAGAGGTGCCAAGAGAATGGCAGTCTTGGGAGACACAGCCTGGGACTTTGGATCAGT |
| | TGGAGGCGCTCTCAACTCATTGGGCAAGGGCATCCATCAAATTTTTGGAGCAGCTTTCAAATCATTG |
| | TTTGGAGGAATGTCCTGGTTCTCACAAATTCTCATTGGAACGTTGCTGATGTGGTTGGGTCTGAACA |
| | CAAAGAATGGATCTATTTCCCTTATGTGCTTGGCCTTAGGGGGAGTGTTGATCTTCTTATCCACAGC |
| | TGTCTCTGCTGATGTGGGGTGCTCGGTGGACTTCTCAAAGAAGGAGACGAGATGCGGTACAGGGGTG |
| | TTCGTCTATAACGACGTTGAAGCCTGGAGGGACAGGTACAAGTACCATCCTGACTCCCCCCGTAGAT |
| | TGGCAGCAGCAGTCAAGCAAGCCTGGGAAGATGGTATCTGTGGGATCTCCTCTGTTTCAAGAATGGA |
| | AAACATCATGTGGAGATCAGTAGAAGGGGAGCTCAACGCAATCCTGGAAGAGAATGGAGTTCAACTG |
| | ACGGTCGTTGTGGGATCTGTAAAAAACCCCATGTGGAGAGGTCCACAGAGATTGCCCGTGCCTGTGA |
| | ACGAGCTGCCCCACGGCTGGAAGGCTTGGGGGAAATCGTACTTCGTCAGAGCAGCAAAGACAAATAA |
| | CAGCTTTGTCGTGGATGGTGACACACTGAAGGAATGCCCACTCAAACATAGAGCATGGAACAGCTTT |
| | CTTGTGGAGGATCATGGGTTCGGGGTATTTCACACTAGTGTCTGGCTCAAGGTTAGAGAAGATTATT |
| | CATTAGAGTGTGATCCAGCCGTTATTGGAACAGCTGTTAAGGGAAAGGAGGCTGTACACAGTGATCT |
| | AGGCTACTGGATTGAGAGTGAGAAGAATGACACATGGAGGCTGAAGAGGGCCCATCTGATCGAGATG |
| | AAAACATGTGAATGGCCAAAGTCCCACACATTGTGGACAGATGGAATAGAAGAGAGTGATCTGATCA |
| | TACCCAAGTCTTTAGCTGGGCCACTCAGCCATCACAATACCAGAGAGGGCTACAGGACCCAAATGAA |
| | AGGGCCATGGCACAGTGAAGAGCTTGAAATTCGGTTTGAGGAATGCCCAGGCACTAAGGTCCACGTG |
| | GAGGAAACATGTGGAACAAGAGGACCATCTCTGAGATCAACCACTGCAAGCGGAAGGGTGATCGAGG |
| | AATGGTGCTGCAGGGAGTGCACAATGCCCCCACTGTCGTTCCGGGCTAAAGATGGCTGTTGGTATGG |
| | AATGGAGATAAGGCCCAGGAAAGAACCAGAAAGTAACTTAGTAAGGTCAATGGTGACTGCAGGATCA |
| | ACTGATCACATGGATCACTTCTCCCTTGGAGTGCTTGTGATTCTGCTCATGGTGCAGGAAGGGCTGA |
| | AGAAGAGAATGACCACAAAGATCATCATAAGCACATCGATGGCAGTGCTGGTAGCTATGATCCTGGG |
| | AGGATTTTCAATGAGTGACCTGGCTAAGCTTGCAATTTTGATGGGTGCCACCTTCGCGGAAATGAAC |
| | ACTGGAGGAGATGTAGCTCATCTGGCGCTGATAGCGGCATTCAAAGTCAGACCAGCGTTGCTGGTAT |
| | CTTTCATCTTCAGAGCTAATTGGACACCCCGTGAAAGCATGCTGCTGGCCTTGGCCTCGTGTCTTTT |
| | GCAAACTGCGATCTCCGCCTTGGAAGGCGACCTGATGGTTCTCATCAATGGTTTTGCTTTGGCCTGG |
| | TTGGCAATACGAGCGATGGTTGTTCCACGCACTGATAACATCACCTTGGCAATCCTGGCTGCTCTGA |
| | CACCACTGGCCCGGGGCACACTGCTTGTGGCGTGGAGAGCAGGCCTTGCTACTTGCGGGGGGTTTAT |
| | GCTCCTCTCTCTGAAGGGAAAAGGCAGTGTGAAGAAGAACTTACCATTTGTCATGGCCCTGGGACTA |
| | ACCGCTGTGAGGCTGGTCGACCCCATCAACGTGGTGGGACTGCTGTTGCTCACAAGGAGTGGGAAGC |
| | GGAGCTGGCCCCCTAGCGAAGTACTCACAGCTGTTGGCCTGATATGCGCATTGGCTGGAGGGTTCGC |
| | CAAGGCAGATATAGAGATGGCTGGGCCCATGGCCGCGGTCGGTCTGCTAATTGTCAGTTACGTGGTC |
| | TCAGGAAAGAGTGTGGAGATGTACATTGAAAGAGCAGGTGACATCACATGGGAAAAAGATGCGGAAG |
| | TCACTGGAAACAGTCCCCGGCTCGATGTGGCGCTAGATGAGAGTGGTGATTTCTCCCTGGTGGAGGA |
| | TGACGGTCCCCCCATGAGAGAGATCATACTCAAGGTGGTCCTGATGACCATCTGTGGCATGAACCCA |
| | ATAGCCATACCCTTTGCAGCTGGAGCGTGGTACGTATACGTGAAGACTGGAAAAAGGAGTGGTGCTC |
| | TATGGGATGTGCCTGCTCCCAAGGAAGTAAAAAAGGGGGAGACCACAGATGGAGTGTACAGAGTAAT |
| | GACTCGTAGACTGCTAGGTTCAACACAAGTTGGAGTGGGAGTTATGCAAGAGGGGGTCTTTCACACT |
| | ATGTGGCACGTCACAAAAGGATCCGCGCTGAGAAGCGGTGAAGGGAGACTTGATCCATACTGGGGAG |
| | ATGTCAAGCAGGATCTGGTGTCATACTGTGGTCCATGGAAGCTAGATGCCGCCTGGGACGGGCACAG |
| | CGAGGTGCAGCTCTTGGCCGTGCCCCCCGGAGAGAGAGCGAGGAACATCCAGACTCTGCCCGGAATA |
| | TTTAAGACAAAGGATGGGGACATTGGAGCGGTTGCGCTGGATTACCCAGCAGGAACTTCAGGATCTC |
| | CAATCCTAGACAAGTGTGGGAGAGTGATAGGACTTTATGGCAATGGGGTCGTGATCAAAAATGGGAG |
| | TTATGTTAGTGCCATCACCCAAGGGAGGAGGGAGGAAGAGACTCCTGTTGAGTGCTTCGAGCCTTCG |
| | ATGCTGAAGAAGAAGCAGCTAACTGTCTTAGACTTGCATCCTGGAGCTGGGAAAACCAGGAGAGTTC |
| | TTCCTGAAATAGTCCGTGAAGCCATAAAAACAAGACTCCGTACTGTGATCTTAGCTCCAACCAGGGT |
| | TGTCGCTGCTGAAATGGAGGAAGCCCTTAGAGGGCTTCCAGTGCGTTATATGACAACAGCAGTCAAT |
| | GTCACCCACTCTGGAACAGAAATCGTCGACTTAATGTGCCATGCCACCTTCACTTCACGTCTACTAC |
| | AGCCAATCAGAGTCCCCAACTATAATCTGTATATTATGGATGAGGCCCACTTCACAGATCCCTCAAG |
| | TATAGCAGCAAGAGGATACATTTCAACAAGGGTTGAGATGGGCGAGGCGGCTGCCATCTTCATGACC |
| | GCCACGCCACCAGGAACCCGTGACGCATTTCCGGACTCCAACTCACCAATTATGGACACCGAAGTGG |
| | AAGTCCCAGAGAGAGCCTGGAGCTCAGGCTTTGATTGGGTGACGGATCATTCTGGAAAAACAGTTTG |
| | GTTTGTTCCAAGCGTGAGGAACGGCAATGAGATCGCAGCTTGTCTGACAAAGGCTGGAAAACGGGTC |
| | ATACAGCTCAGCAGAAAGACTTTTGAGACAGAGTTCCAGAAAACAAAACATCAAGAGTGGGACTTTG |
| | TCGTGACAACTGACATTTCAGAGATGGGCGCCAACTTTAAAGCTGACCGTGTCATAGATTCCAGGAG |
| | ATGCCTAAAGCCGGTCATACTTGATGGCGAGAGAGTCATTCTGGCTGGACCCATGCCTGTCACACAT |
| | GCCAGCGCTGCCCAGAGGAGGGGGCGCATAGGCAGGAATCCCAACAAACCTGGAGATGAGTATCTGT |
| | ATGGAGGTGGGTGCGCAGAGACTGACGAAGACCATGCACACTGGCTTGAAGCAAGAATGCTCCTTGA |
| | CAATATTTACCTCCAAGATGGCCTCATAGCCTCGCTCTATCGACCTGAGGCCGACAAAGTAGCAGCC |
| | ATTGAGGGAGAGTTCAAGCTTAGGACGGAGCAAAGGAAGACCTTTGTGGAACTCATGAAAAGAGGAG |
| | ATCTTCCTGTTTGGCTGGCCTATCAGGTTGCATCTGCCGGAATAACCTACACAGATAGAAGATGGTG |
| | CTTTGATGGCACGACCAACAACACCATAATGGAAGACAGTGTGCCGGCAGAGGTGTGGACCAGACAC |
| | GGAGAGAAAAGAGTGCTCAAACCGAGGTGGATGGACGCCAGAGTTTGTTCAGATCATGCGGCCCTGA |
| | AGTCATTCAAGGAGTTTGCCGCTGGGAAAAGAGGAGCGGCTTTTGGAGTGATGGAAGCCCTGGGAAC |
| | ACTGCCAGGACACATGACAGAGAGATTCCAGGAAGCCATTGACAACCTCGCTGTGCTCATGCGGGCA |
| | GAGACTGGAAGCAGGCCTTACAAAGCCGCGGCGGCCCAATTGCCGGAGACCCTAGAGACCATTATGC |
| | TTTTGGGGTTGCTGGGAACAGTCTCGCTGGGAATCTTTTTCGTCTTGATGAGGAACAAGGGCATAGG |
| | GAAGATGGGCTTTGGAATGGTGACTCTTGGGGCCAGCGCATGGCTCATGTGGCTCTCGGAAATTGAG |
| | CCAGCCAGAATTGCATGTGTCCTCATTGTTGTGTTCCTATTGCTGGTGGTGCTCATACCTGAGCCAG |
| | AAAAGCAAAGATCTCCCCAGGACAACCAAATGGCAATCATCATCATGGTAGCAGTAGGTCTTCTGGG |
| | CTTGATTACCGCCAATGAACTCGGATGGTTGGAGAGAACAAAGAGTGACCTAAGCCATCTAATGGGA |
| | AGGAGAGAGGAGGGGGCAACCATAGGATTCTCAATGGACATTGACCTGCGGCCAGCCTCAGCTTGGG |
| | CCATCTATGCTGCCTTGACAACTTTCATTACCCCAGCCGTCCAACATGCAGTGACCACTTCATACAA |
| | CAACTACTCCTTAATGGCGATGGCCACGCAAGCTGGAGTGTTGTTTGGTATGGGCAAAGGGATGCCA |
| | TTCTACGCATGGGACTTTGGAGTCCCGCTGCTAATGATAGGTTGCTACTCACAATTAACACCCCTGA |
| | CCCTAATAGTGGCCATCATTTTGCTCGTGGCGCACTACATGTACTTGATCCCAGGGCTGCAGGCAGC |
| | AGCTGCGCGTGCTGCCCAGAAGAGAACGGCAGCTGGCATCATGAAGAACCCTGTTGTGGATGGAATA |
| | GTGGTGACTGACATTGACACAATGACAATTGACCCCCAAGTGGAGAAAAAGATGGGACAGGTGCTAC |
| | TCATAGCAGTAGCCGTCTCCAGCGCCATACTGTCGCGGACCGCCTGGGGGTGGGGGGAGGCTGGGGC |
| | CCTGATCACAGCGGCAACTTCCACTTTGTGGGAAGGCTCTCCGAACAAGTACTGGAACTCCTCTACA |
| | GCCACTTCACTGTGTAACATTTTTAGGGGAAGTTACTTGGCTGGAGCTTCTCTAATCTACACAGTAA |
| | CAAGAAACGCTGGCTTGGTCAAGAGACGTGGGGGTGGAACAGGAGAGACCCTGGGAGAGAAATGGAA |
| | GGCCCGCTTGAACCAGATGTCGGCCCTGGAGTTCTACTCCTACAAAAAGTCAGGCATCACCGAGGTG |
| | TGCAGAGAAGAGGCCCGCCGCGCCCTCAAGGACGGTGTGGCAACGGGAGGCCATGCTGTGTCCCGAG |
| | GAAGTGCAAAGCTGAGATGGTTGGTGGAGCGGGGATACCTGCAGCCCTATGGAAAGGTCATTGATCT |
| | TGGATGTGGCAGAGGGGGCTGGAGTTACTACGCCGCCACCATCCGCAAAGTTCAAGAAGTGAAAGGA |
| | TACACAAAAGGAGGCCCTGGTCATGAAGAACCCATGTTGGTGCAAAGCTATGGGTGGAACATAGTCC |
| | GTCTTAAGAGTGGGGTGGACGTCTTTCATATGGCGGCTGAGCCGTGTGACACGTTGCTGTGTGACAT |
| | AGGTGAGTCATCATCTAGTCCTGAAGTGGAAGAAGCACGGACGCTCAGAGTCCTCTCCATGGTGGGG |
| | GATTGGCTTGAAAAAAGACCAGGAGCCTTTTGTATAAAAGTGTTGTGCCCATACACCAGCACTATGA |
| | TGGAAACCCTGGAGCGACTGCAGCGTAGGTATGGGGGAGGACTGGTCAGAGTGCCACTCTCCCGCAA |
| | CTCTACACATGAGATGTACTGGGTCTCTGGAGCGAAAAGCAACACCATAAAAAGTGTGTCCACCACG |
| | AGCCAGCTCCTCTTGGGGCGCATGGACGGGCCCAGGAGGCCAGTGAAATATGAGGAGGATGTGAATC |
| | TCGGCTCTGGCACGCGGGCTGTGGTAAGCTGCGCTGAAGCTCCCAACATGAAGATCATTGGTAACCG |
| | CATTGAAAGGATCCGCAGTGAGCACGCGGAAACGTGGTTCTTTGACGAGAACCACCCATATAGGACA |
| | TGGGCTTACCATGGAAGCTATGAGGCCCCCACACAAGGGTCAGCGTCCTCTCTAATAAACGGGGTTG |
| | TCAGGCTCCTGTCAAAACCCTGGGATGTGGTGACTGGAGTCACAGGAATAGCCATGACCGACACCAC |
| | ACCGTATGGTCAGCAAAGAGTTTTCAAGGAAAAAGTGGACACTAGGGTGCCAGACCCCCAAGAAGGC |
| | ACTCGTCAGGTTATGAGCATGGTCTCTTCCTGGTTGTGGAAAGAGCTAGGCAAACACAAACGGCCAC |
| | GAGTCTGTACCAAAGAAGAGTTCATCAACAAGGTTCGTAGCAATGCAGCATTAGGGGCAATATTTGA |
| | AGAGGAAAAAGAGTGGAAGACTGCAGTGGAAGCTGTGAAGGATCCAAGGTTCTGGGCTCTAGTGGAC |
| | AAGGAAAGAGAGCACCACCTGAGAGGAGAGTGCCAGAGTTGTGTGTACAACATGATGGGAAAAAGAG |
| | AAAAGAAACAAGGGGAATTTGGAAAGGCCAAGGGCAGCCGCGCCATCTGGTATATGTGGCTAGGGGC |
| | TAGATTTCTAGAGTTCGAAGCCCTTGGATTCTTGAACGAGGATCACTGGATGGGGAGAGAGAACTCA |
| | GGAGGTGGTGTTGAAGGGCTGGGATTACAAAGACTCGGATATGTCCTAGAAGAGATGAGTCGCATAC |
| | CAGGAGGAAGGATGTATGCAGATGACACTGCTGGCTGGGACACCCGCATCAGCAGGTTTGATCTGGA |
| | GAATGAAGCTCTAATCACCAACCAAATGGAGAAAGGGCACAGGGCCTTGGCATTGGCCATAATCAAG |
| | TACACATACCAAAACAAAGTGGTAAAGGTCCTTAGACCAGCTGAAAAAGGGAAGACAGTTATGGACA |
| | TTATTTCGAGACAAGACCAAAGGGGGAGCGGACAAGTTGTCACTTACGCTCTTAACACATTTACCAA |
| | CCTAGTGGTGCAACTCATTCGGAATATGGAGGCTGAGGAAGTTCTAGAGATGCAAGACTTGTGGCTG |
| | CTGCGGAGGTCAGAGAAAGTGACCAACTGGTTGCAGAGCAACGGATGGGATAGGCTCAAACGAATGG |
| | CAGTCAGTGGAGATGATTGCGTTGTGAAGCCAATTGATGATAGGTTTGCACATGCCCTCAGGTTCTT |
| | GAATGATATGGGAAAAGTTAGGAAGGACACACAAGAGTGGAAACCCTCAACTGGATGGGACAACTGG |
| | GAAGAAGTTCCGTTTTGCTCCCACCACTTCAACAAGCTCCATCTCAAGGACGGGAGGTCCATTGTGG |
| | TTCCCTGCCGCCACCAAGATGAACTGATTGGCCGGGCCCGCGTCTCTCCAGGGGCGGGATGGAGCAT |
| | CCGGGAGACTGCTTGCCTAGCAAAATCATATGCGCAAATGTGGCAGCTCCTTTATTTCCACAGAAGG |
| | GACCTCCGACTGATGGCCAATGCCATTTGTTCATCTGTGCCAGTTGACTGGGTTCCAACTGGGAGAA |
| | CTACCTGGTCAATCCATGGAAAGGGAGAATGGATGACCACTGAAGACATGCTTGTGGTGTGGAACAG |
| | AGTGTGGATTGAGGAGAACGACCACATGGAAGACAAGACCCCAGTTACGAAATGGACAGACATTCCC |
| | TATTTGGGAAAAAGGGAAGACTTGTGGTGTGGATCTCTCATAGGGCACAGACCGCGCACCACCTGGG |
| | CTGAGAACATTAAAAACACAGTCAACATGGTGCGCAGGATCATAGGTGATGAAGAAAAGTACATGGA |
| | CTACCTATCCACCCAAGTTCGCTACTTGGGTGAAGAAGGGTCTACACCTGGAGTGCTGTAAGCACCA |
| | ATCTTAGTGTTGTCAGGCCTGCTAGTCAGCCACAGCTTGGGGAAAGCTGTGCAGCCTGTGACCCCCC |
| | CAGGAGAAGCTGGGAAACCAAGCCTATAGTCAGGCCGAGAACGCCATGGCACGGAAGAAGCCATGCT |
| | GCCTGTGAGCCCCTCAGAGGACACTGAGTCAAAAAACCCCACGCGCTTGGAGGCGCAGGATGGGAAA |
| | AGAAGGTGGCGACCTTCCCCACCCTTCAATCTGGGGCCTGAACTGGAGATCAGCTGTGGATCTCCAG |
| | AAGAGGGACTAGTGGTTAGAGGAGACCCCCCGGAAAACGCAAAACAGCATATTGACGCTGGGAAAGA |
| | CCAGAGACTCCATGAGTTTCCACCACGCTGGCCGCCAGGCACAGATCGCCGAATAGCGGCGGCCGGT |
| | GTGGGGAAATCCATGGGTCT |
| 299 | GenBank Accession No. KX377337.1 (Zika virus strain PRVABC-59, complete |
| | genome) |
| | AGTTGTTGATCTGTGTGAATCAGACTGCGACAGTTCGAGTTTGAAGCGAAAGCTAGCAACAGTATCA |
| | ACAGGTTTTATTTTGGATTTGGAAACGAGAGTTTCTGGTCATGAAAAACCCAAAAAAGAAATCCGGA |
| | GGATTCCGGATTGTCAATATGCTAAAACGCGGAGTAGCCCGTGTGAGCCCCTTTGGGGGCTTGAAGA |
| | GGCTGCCAGCCGGACTTCTGCTGGGTCATGGGCCCATCAGGATGGTCTTGGCGATTCTAGCCTTTTT |
| | GAGATTCACGGCAATCAAGCCATCACTGGGTCTCATCAATAGATGGGGTTCAGTGGGGAAAAAAGAG |
| | GCTATGGAAACAATAAAGAAGTTCAAGAAAGATCTGGCTGCCATGCTGAGAATAATCAATGCTAGGA |
| | AGGAGAAGAAGAGACGAGGCGCAGATACTAGTGTCGGAATTGTTGGCCTCCTGCTGACCACAGCTAT |
| | GGCAGCGGAGGTCACTAGACGTGGGAGTGCATACTATATGTACTTGGACAGAAACGATGCTGGGGAG |
| | GCCATATCTTTTCCAACCACATTGGGGATGAATAAGTGTTATATACAGATCATGGATCTTGGACACA |
| | TGTGTGATGCCACCATGAGCTATGAATGCCCTATGCTGGATGAGGGGGTGGAACCAGATGACGTCGA |
| | TTGTTGGTGCAACACGACGTCAACTTGGGTTGTGTACGGAACCTGCCATCACAAAAAAGGTGAAGCA |
| | CGGAGATCTAGAAGAGCTGTGACGCTCCCCTCCCATTCCACCAGGAAGCTGCAAACGCGGTCGCAAA |
| | CCTGGTTGGAATCAAGAGAATACACAAAGCACTTGATTAGAGTCGAAAATTGGATATTCAGGAACCC |
| | TGGCTTCGCGTTAGCAGCAGCTGCCATCGCTTGGCTTTTGGGAAGCTCAACGAGCCAAAAAGTCATA |
| | TACTTGGTCATGATACTGCTGATTGCCCCGGCATACAGCATCAGGTGCATAGGAGTCAGCAATAGGG |
| | ACTTTGTGGAAGGTATGTCAGGTGGGACTTGGGTTGATGTTGTCTTGGAACATGGAGGTTGTGTCAC |
| | CGTAATGGCACAGGACAAACCGACTGTCGACATAGAGCTGGTTACAACAACAGTCAGCAACATGGCG |
| | GAGGTAAGATCCTACTGCTATGAGGCATCAATATCAGACATGGCTTCTGACAGCCGCTGCCCAACAC |
| | AAGGTGAAGCCTACCTTGACAAGCAATCAGACACTCAATATGTCTGCAAAAGAACGTTAGTGGACAG |
| | AGGCTGGGGAAATGGATGTGGACTTTTTGGCAAAGGGAGCCTGGTGACATGCGCTAAGTTTGCATGC |
| | TCCAAGAAAATGACCGGGAAGAGCATCCAGCCAGAGAATCTGGAGTACCGGATAATGCTGTCAGTTC |
| | ATGGCTCCCAGCACAGTGGGATGATCGTTAATGACACAGGACATGAAACTGATGAGAATAGAGCGAA |
| | AGTTGAGATAACGCCCAATTCACCGAGAGCCGAAGCCACCCTGGGGGGTTTTGGAAGCCTAGGACTT |
| | GATTGTGAACCGAGGACAGGCCTTGACTTTTCAGATTTGTATTACTTGACTATGAATAACAAGCACT |
| | GGTTGGTTCACAAGGAGTGGTTCCACGACATTCCATTACCTTGGCACGCTGGGGCAGACACCGGAAC |
| | TCCACACTGGAACAACAAAGAAGCACTGGTAGAGTTCAAGGACGCACATGCCAAAAGGCAAACTGTC |
| | GTGGTTCTAGGGAGTCAAGAAGGAGCAGTTCACACGGCCCTTGCTGGAGCTCTGGAGGCTGAGATGG |
| | ATGGTGCAAAGGGAAGGCTGTCCTCTGGCCACTTGAAATGTCGCCTGAAAATGGATAAACTTAGATT |
| | GAAGGGCGTGTCATACTCCTTGTGTACTGCAGCGTTCACATTCACCAAGATCCCGGCTGAAACACTG |
| | CACGGGACAGTCACAGTGGAGGTACAGTACGCAGGGACAGATGGACCTTGCAAGGTTCCAGCTCAGA |
| | TGGCGGTGGACATGCAAACTCTGACCCCAGTTGGGAGGTTGATAACCGCTAACCCCGTAATCACTGA |
| | AAGCACTGAGAACTCTAAGATGATGCTGGAACTTGATCCACCATTTGGGGACTCTTACATTGTCATA |
| | GGAGTCGGGGAGAAGAAGATCACCCACCACTGGCACAGGAGTGGCAGCACCATTGGAAAAGCATTTG |
| | AAGCCACTGTGAGAGGTGCCAAGAGAATGGCAGTCTTGGGAGACACAGCCTGGGACTTTGGATCAGT |
| | TGGAGGCGCTCTCAACTCATTGGGCAAGGGCATCCATCAAATTTTTGGAGCAGCTTTCAAATCATTG |
| | TTTGGAGGAATGTCCTGGTTCTCACAAATTCTCATTGGAACGTTGCTGATGTGGTTGGGTCTGAACA |
| | CAAAGAATGGATCTATTTCCCTTATGTGCTTGGCCTTAGGGGGAGTGTTGATCTTCTTATCCACAGC |
| | CGTCTCTGCTGATGTGGGGTGCTCGGTGGACTTCTCAAAGAAGGAGACGAGATGCGGTACAGGGGTG |
| | TTCGTCTATAACGACGTTGAAGCCTGGAGGGACAGGTACAAGTACCATCCTGACTCCCCCCGTAGAT |
| | TGGCAGCAGCAGTCAAGCAAGCCTGGGAAGATGGTATCTGCGGGATCTCCTCTGTTTCAAGAATGGA |
| | AAACATCATGTGGAGATCAGTAGAAGGGGAGCTCAACGCAATCCTGGAAGAGAATGGAGTTCAACTG |
| | ACGGTCGTTGTGGGATCTGTAAAAAACCCCATGGGGAGAGGTCCACAGAGATTGCCCGTGCCTGTGA |
| | ACGAGCTGCCCCACGGCTGGAAGGCTTGGGGGAAATCGTATTTCGTCAGAGCAGCAAAGACAAATAA |
| | CAGCTTTGTCGTGGATGGTGACACACTGAAGGAATGCCCACTCAAACATAGAGCATGGAACAGCTTT |
| | CTTGTGGAGGATCATGGGTTCGGGGTATTTCACACTAGTGTCTGGCTCAAGGTTAGAGAAGATTATT |
| | CATTAGAGTGTGATCCAGCCGTTATTGGAACAGCTGTTAAGGGAAAGGAGGCTGTACACAGTGATCT |
| | AGGCTACTGGATTGAGAGTGAGAAGAATGACACATGGAGGCTGAAGAGGGCCCATCTGATCGAGATG |
| | AAAACATGTGAATGGCCAAAGTCCCACACATTGTGGACAGATGGAATAGAAGAGAGTGATCTGATCA |
| | TACCCAAGTCTTTAGCTGGGCCACTCAGCCATCACAATACCAGAGAGGGCTACAGGACCCAAATGAA |
| | AGGGCCATGGCACAGTGAAGAGCTTGAAATTCGGTTTGAGGAATGCCCAGGCACTAAGGTCCACGTG |
| | GAGGAAACATGTGGAACAAGAGGACCATCTCTGAGATCAACCACTGCAAGCGGAAGGGTGATCGAGG |
| | AATGGTGCTGCAGGGAGTGCACAATGCCCCCACTGTCGTTCCGGGCTAAAGATGGCTGTTGGTATGG |
| | AATGGAGATAAGGCCCAGGAAAGAACCAGAAAGCAACTTAGTAAGGTCAATGGTGACTGCAGGATCA |
| | ACTGATCACATGGACCACTTCTCCCTTGGAGTGCTTGTGATCCTGCTCATGGTGCAGGAAGGGCTGA |
| | AGAAGAGAATGACCACAAAGATCATCATAAGCACATCAATGGCAGTGCTGGTAGCTATGATCCTGGG |
| | AGGATTTTCAATGAGTGACCTGGCTAAGCTTGCAATTTTGATGGGTGCCACCTTCGCGGAAATGAAC |
| | ACTGGAGGAGATGTAGCTCATCTGGCGCTGATAGCGGCATTCAAAGTCAGACCAGCGTTGCTGGTAT |
| | CTTTCATCTTCAGAGCTAATTGGACACCCCGTGAAAGCATGCTGCTGGCCTTGGCCTCGTGTCTTTT |
| | GCAAACTGCGATCTCCGCCTTGGAAGGCGACCTGATGGTTCTCATCAATGGTTTTGCTTTGGCCTGG |
| | TTGGCAATACGAGCGATGGTTGTTCCACGCACTGATAACATCACCTTGGCAATCCTGGCTGCTCTGA |
| | CACCACTGGCCCGGGGCACACTGCTTGTGGCGTGGAGAGCAGGCCTTGCTACTTGCGGGGGGTTTAT |
| | GCTCCTCTCTCTGAAGGGAAAAGGCAGTGTGAAGAAGAACTTACCATTTGTCATGGCCCTGGGACTA |
| | ACCGCTGTGAGGCTGGTCGACCCCATCAACGTGGTGGGACTGCTGTTGCTCACAAGGAGTGGGAAGC |
| | GGAGCTGGCCCCCTAGCGAAGTACTCACAGCTGTTGGCCTGATATGCGCATTGGCTGGAGGGTTCGC |
| | CAAGGCAGATATAGAGATGGCTGGGCCCATGGCCGCGGTCGGTCTGCTAATTGTCAGTTACGTGGTC |
| | TCAGGAAAGAGTGTGGAGATGTACATTGAAAGAGCAGGTGACATCACATGGGAAAAAGATGCGGAAG |
| | TCACTGGAAACAGTCCCCGGCTCGATGTGGCGCTAGATGAGAGTGGTGATTTCTCCCTGGTGGAGGA |
| | TGACGGTCCCCCCATGAGAGAGATCATACTCAAGGTGGTCCTGATGACCATCTGTGGCATGAACCCA |
| | ATAGCCATACCCTTTGCAGCTGGAGCGTGGTACGTATACGTGAAGACTGGAAAAAGGAGTGGTGCTC |
| | TATGGGATGTGCCTGCTCCCAAGGAAGTAAAAAAGGGGGAGACCACAGATGGAGTGTACAGAGTAAT |
| | GACTCGTAGACTGCTAGGTTCAACACAAGTTGGAGTGGGAGTTATGCAAGAGGGGGTCTTTCACACT |
| | ATGTGGCACGTCACAAAAGGATCCGCGCTGAGAAGCGGTGAAGGGAGACTTGATCCATACTGGGGAG |
| | ATGTCAAGCAGGATCTGGTGTCATACTGTGGTCCATGGAAGCTAGATGCCGCCTGGGATGGGCACAG |
| | CGAGGTGCAGCTCTTGGCCGTGCCCCCCGGAGAGAGAGCGAGGAACATCCAGACTCTGCCCGGAATA |
| | TTTAAGACAAAGGATGGGGACATTGGAGCGGTTGCGCTGGATTACCCAGCAGGAACTTCAGGATCTC |
| | CAATCCTAGACAAGTGTGGGAGAGTGATAGGACTTTATGGCAATGGGGTCGTGATCAAAAACGGGAG |
| | TTATGTTAGTGCCATCACCCAAGGGAGGAGGGAGGAAGAGACTCCTGTTGAGTGCTTCGAGCCCTCG |
| | ATGCTGAAGAAGAAGCAGCTAACTGTCTTAGACTTGCATCCTGGAGCTGGGAAAACCAGGAGAGTTC |
| | TTCCTGAAATAGTCCGTGAAGCCATAAAAACAAGACTCCGTACTGTGATCTTAGCTCCAACCAGGGT |
| | TGTCGCTGCTGAAATGGAGGAGGCCCTTAGAGGGCTTCCAGTGCGTTATATGACAACAGCAGTCAAT |
| | GTCACCCACTCTGGAACAGAAATCGTCGACTTAATGTGCCATGCCACCTTCACTTCACGTCTACTAC |
| | AGCCAATCAGAGTCCCCAACTATAATCTGTATATTATGGATGAGGCCCACTTCACAGATCCCTCAAG |
| | TATAGCAGCAAGAGGATACATTTCAACAAGGGTTGAGATGGGCGAGGCGGCTGCCATCTTCATGACC |
| | GCCACGCCACCAGGAACCCGTGACGCATTTCCGGACTCCAACTCACCAATTATGGACACCGAAGTGG |
| | AAGTCCCAGAGAGAGCCTGGAGCTCAGGCTTTGATTGGGTGACGGATCATTCTGGAAAAACAGTTTG |
| | GTTTGTTCCAAGCGTGAGGAACGGCAATGAGATCGCAGCTTGTCTGACAAAGGCTGGAAAACGGGTC |
| | ATACAGCTCAGCAGAAAGACTTTTGAGACAGAGTTCCAGAAAACAAAACATCAAGAGTGGGACTTTG |
| | TCGTGACAACTGACATTTCAGAGATGGGCGCCAACTTTAAAGCTGACCGTGTCATAGATTCCAGGAG |
| | ATGCCTAAAGCCGGTCATACTTGATGGCGAGAGAGTCATTCTGGCTGGACCCATGCCTGTCACACAT |
| | GCCAGCGCTGCCCAGAGGAGGGGGCGCATAGGCAGGAATCCCAACAAACCTGGAGATGAGTATCTGT |
| | ATGGAGGTGGGTGCGCAGAGACTGACGAAGACCATGCACACTGGCTTGAAGCAAGAATGCTCCTTGA |
| | CAATATTTACCTCCAAGATGGCCTCATAGCCTCGCTCTATCGACCTGAGGCCGACAAAGTAGCAGCC |
| | ATTGAGGGAGAGTTCAAGCTTAGGACGGAGCAAAGGAAGACCTTTGTGGAACTCATGAAAAGAGGAG |
| | ATCTTCCTGTTTGGCTGGCCTATCAGGTTGCATCTGCCGGAATAACCTACACAGATAGAAGATGGTG |
| | CTTTGATGGCACGACCAACAACACCATAATGGAAGACAGTGTGCCGGCAGAGGTGTGGACCAGACAC |
| | GGAGAGAAAAGAGTGCTCAAACCGAGGTGGATGGACGCCAGAGTTTGTTCAGATCATGCGGCCCTGA |
| | AGTCATTCAAGGAGTTTGCCGCTGGGAAAAGAGGAGCGGCTTTTGGAGTGATGGAAGCCCTGGGAAC |
| | ACTGCCAGGACACATGACAGAGAGATTCCAGGAAGCCATTGACAACCTCGCTGTGCTCATGCGGGCA |
| | GAGACTGGAAGCAGGCCTTACAAAGCCGCGGCGGCCCAATTGCCGGAGACCCTAGAGACCATAATGC |
| | TTTTGGGGTTGCTGGGAACAGTCTCGCTGGGAATCTTCTTCGTCTTGATGAGGAACAAGGGCATAGG |
| | GAAGATGGGCTTTGGAATGGTGACTCTTGGGGCCAGCGCATGGCTCATGTGGCTCTCGGAAATTGAG |
| | CCAGCCAGAATTGCATGTGTCCTCATTGTTGTGTTCCTATTGCTGGTGGTGCTCATACCTGAGCCAG |
| | AAAAGCAAAGATCTCCCCAGGACAACCAAATGGCAATCATCATCATGGTAGCAGTAGGTCTTCTGGG |
| | CTTGATTACCGCCAATGAACTCGGATGGTTGGAGAGAACAAAGAGTGACCTAAGCCATCTAATGGGA |
| | AGGAGAGAGGAGGGGGCAACCATAGGATTCTCAATGGACATTGACCTGCGGCCAGCCTCAGCTTGGG |
| | CCATCTATGCTGCCTTGACAACTTTCATTACCCCAGCCGTCCAACATGCAGTGACCACCTCATACAA |
| | CAACTACTCCTTAATGGCGATGGCCACGCAAGCTGGAGTGTTGTTTGGCATGGGCAAAGGGATGCCA |
| | TTCTACGCATGGGACTTTGGAGTCCCGCTGCTAATGATAGGTTGCTACTCACAATTAACACCCCTGA |
| | CCCTAATAGTGGCCATCATTTTGCTCGTGGCGCACTACATGTACTTGATCCCAGGGCTGCAGGCAGC |
| | AGCTGCGCGTGCTGCCCAGAAGAGAACGGCAGCTGGCATCATGAAGAACCCTGTTGTGGATGGAATA |
| | GTGGTGACTGACATTGACACAATGACAATTGACCCCCAAGTGGAGAAAAAGATGGGACAGGTGCTAC |
| | TCATAGCAGTAGCCGTCTCCAGCGCCATACTGTCGCGGACCGCCTGGGGGTGGGGGGAGGCTGGGGC |
| | TCTGATCACAGCCGCAACTTCCACTTTGTGGGAAGGCTCTCCGAACAAGTACTGGAACTCCTCTACA |
| | GCCACTTCACTGTGTAACATTTTTAGGGGAAGTTACTTGGCTGGAGCTTCTCTAATCTACACAGTAA |
| | CAAGAAACGCTGGCTTGGTCAAGAGACGTGGGGGTGGAACAGGAGAGACCCTGGGAGAGAAATGGAA |
| | GGCCCGCTTGAACCAGATGTCGGCCCTGGAGTTCTACTCCTACAAAAAGTCAGGCATCACCGAGGTG |
| | TGCAGAGAAGAGGCCCGCCGCGCCCTCAAGGACGGTGTGGCAACGGGAGGCCATGCTGTGTCCCGAG |
| | GAAGTGCAAAGCTGAGATGGTTGGTGGAGCGGGGATACCTGCAGCCCTATGGAAAGGTCATTGATCT |
| | TGGATGTGGCAGAGGGGGCTGGAGTTACTACGTCGCCACCATCCGCAAAGTTCAAGAAGTGAAAGGA |
| | TACACAAAAGGAGGCCCTGGTCATGAAGAACCCGTGTTGGTGCAAAGCTATGGGTGGAACATAGTCC |
| | GTCTTAAGAGTGGGGTGGACGTCTTTCATATGGCGGCTGAGCCGTGTGACACGTTGCTGTGTGACAT |
| | AGGTGAGTCATCATCTAGTCCTGAAGTGGAAGAAGCACGGACGCTCAGAGTCCTCTCCATGGTGGGG |
| | GATTGGCTTGAAAAAAGACCAGGAGCCTTTTGTATAAAAGTGTTGTGCCCATACACCAGCACTATGA |
| | TGGAAACCCTGGAGCGACTGCAGCGTAGGTATGGGGGAGGATTGGTCAGAGTGCCACTCTCCCGCAA |
| | CTCTACACATGAGATGTACTGGGTCTCTGGAGCGAAAAGCAACACCATAAAAAGTGTGTCCACCACG |
| | AGCCAGCTCCTCTTGGGGCGCATGGACGGGCCTAGGAGGCCAGTGAAATATGAGGAGGATGTGAATC |
| | TCGGCTCTGGCACGCGGGCTGTGGTAAGCTGCGCTGAAGCTCCCAACATGAAGATCATTGGTAACCG |
| | CATTGAAAGGATCCGCAGTGAGCACGCGGAAACGTGGTTCTTTGACGAGAACCACCCATATAGGACA |
| | TGGGCTTACCATGGAAGCTATGAGGCCCCCACACAAGGGTCAGCGTCCTCTCTAATAAACGGGGTTG |
| | TCAGGCTCCTGTCAAAACCCTGGGATGTGGTGACTGGAGTCACAGGAATAGCCATGACCGACACCAC |
| | ACCGTATGGTCAGCAAAGAGTTTTCAAGGAAAAAGTGGACACTAGGGTGCCAGACCCCCAAGAAGGC |
| | ACTCGTCAGGTTATGAGCATGGTCTCTTCCTGGTTGTGGAAAGAGCTAGGCAAACACAAACGGCCAC |
| | GAGTCTGCACCAAAGAAGAGTTCATCAACAAGGTTCGTAGCAATGCAGCATTAGGGGCAATATTTGA |
| | AGAGGAAAAAGAGTGGAAGACTGCAGTGGAAGCTGTGAAGGATCCAAGGTTCTGGGCTCTAGTGGAC |
| | AAGGAAAGAGAGCACCACCTGAGAGGAGAGTGCCAGAGCTGTGTGTACAACATGATGGGAAAAAGAG |
| | AAAAGAAACAAGGGGAATTTGGAAAGGCCAAGGGCAGCCGCGCCATCTGGTATATGTGGCTAGGGGC |
| | TAGATTTCTAGAGTTCGAAGCCCTTGGATTCTTGAACGAGGATCACTGGATGGGGAGAGAGAACTCA |
| | GGAGGTGGTGTTGAAGGGCTGGGATTACAAAGACTCGGATATGTCCTAGAAGAGATGAGTCGTATAC |
| | CAGGAGGAAGGATGTATGCAGATGACACTGCTGGCTGGGACACCCGCATTAGCAGGTTTGATCTGGA |
| | GAATGAAGCTCTAATCACCAACCAAATGGAGAAAGGGCACAGGGCCTTGGCATTGGCCATAATCAAG |
| | TACACATACCAAAACAAAGTGGTAAAGGTCCTTAGACCAGCTGAAAAAGGGAAAACAGTTATGGACA |
| | TTATTTCGAGACAAGACCAAAGGGGGAGCGGACAAGTTGTCACTTACGCTCTTAACACATTTACCAA |
| | CCTAGTGGTGCAACTCATTCGGAATATGGAGGCTGAGGAAGTTCTAGAGATGCAAGACTTGTGGCTG |
| | CTGCGGAGGTCAGAGAAAGTGACCAACTGGTTGCAGAGCAACGGATGGGATAGGCTCAAACGAATGG |
| | CAGTCAGTGGAGATGATTGCGTTGTGAAGCCAATTGATGATAGGTTTGCACATGCCCTCAGGTTCTT |
| | GAATGATATGGGAAAAGTTAGGAAGGACACACAAGAGTGGAAACCCTCAACTGGATGGGACAACTGG |
| | GAAGAAGTTCCGTTTTGCTCCCACCACTTCAACAAGCTCCATCTCAAGGACGGGAGGTCCATTGTGG |
| | TTCCCTGCCGCCACCAAGATGAACTGATTGGCCGGGCCCGCGTCTCTCCAGGGGCGGGATGGAGCAT |
| | CCGGGAGACTGCTTGCCTAGCAAAATCATATGCGCAAATGTGGCAGCTCCTTTATTTCCACAGAAGG |
| | GACCTCCGACTGATGGCCAATGCCATTTGTTCATCTGTGCCAGTTGACTGGGTTCCAACTGGGAGAA |
| | CTACCTGGTCAATCCATGGAAAGGGAGAATGGATGACCACTGAAGACATGCTTGTGGTGTGGAACAG |
| | AGTGTGGATTGAGGAGAACGACCACATGGAAGACAAGACCCCAGTTACGAAATGGACAGACATTCCC |
| | TATTTGGGAAAAAGGGAAGACTTGTGGTGTGGATCTCTCATAGGGCACAGACCGCGCACCACCTGGG |
| | CTGAGAACATTAAAAACACAGTCAACATGGTGCGCAGGATCATAGGTGATGAAGAAAAGTACATGGA |
| | CTACCTATCCACCCAAGTTCGCTACTTGGGTGAAGAAGGGTCTACACCTGGAGTGCTGTAAGCACCA |
| | ATCTTAATGTTGTCAGGCCTGCTAGTCAGCCACAGCTTGGGGAAAGCTGTGCAGCCTGTGACCCCCC |
| | CAGGAGAAGCTGGGAAACCAAGCCTATAGTCAGGCCGAGAACGCCATGGCACGGAAGAAGCCATGCT |
| | GCCTGTGAGCCCCTCAGAGGACACTGAGTCAAAAAACCCCACGCGCTTGGAGGCGCAGGATGGGAAA |
| | AGAAGGTGGCGACCTTCCCCACCCTTCAATCTGGGGCCTGAACTGGAGATCAGCTGTGGATCTCCAG |
| | AAGAGGGACTAGTGGTTAGAGGAGACCCCCCGGAAAACGCAAAACAGCATATTGACGCTGGGAAAGA |
| | CCAGAGACTCCATGAGTTTCCACCACGCTGGCCGCCAGGCACAGATCGCCGAATAGCGGCGGCCGGT |
| | GTGGGGAAATCCATGGGTCT |
| 294 | GenBank Accession No. KY766069.1 (Zika virus isolate Pf13/251013-18, |
| | complete genome) |
| | CTGTGTGAATCAGACTGCGACAGTTCGAGTTTGAAGCGAAAGCTGCAACAGTTATCAACAGGTTTTA |
| | TTTTGGATTTGGAAACGAGAGTTTCTGGTCATGAAAAACCCAAAAAAGAAATCCGGAGGATTCCGGA |
| | TTGTCAATATGCTAAAACGCGGAGTAGCCCGTGTGAGCCCCTTTGGGGGCTTGAAGAGGCTGCCAGC |
| | CGGACTTCTGCTGGGTCATGGGCCCATCAGGATGGTCTTGGCGATTCTAGCCTTTTTGAGATTCACG |
| | GCAATCAAGGCATCACTGGGTGTCATCAATAGATGGGGTTCAGTGGGGAAAAAAGAGGCTATGGAAA |
| | TAATAAAGAAGTTCAAGAAAGATCTGGCTGCCATGCTGAGAATAATCAATGCTAGGAAGGAGAAGAA |
| | GAGACGAGGCGCAGATACTAGTGTCGGAATTGTTGGCCTCCTGCTGACCACAGCTATGGCAGCGGAG |
| | GTCACTAGACGTGGGAGTGCATACTATATGTACTTGGACAGAAACGATGCTGGGGAGGCCATATCTT |
| | TTCCAACCACATTGGGGATGAATAAGTGTTATATACAGATCATGGATCTTGGACACATGTGTGATGC |
| | CACCATGAGCTATGAATGCCCTATGCTGGATGAGGGGGTGGAACCAGATGACGTCGATTGTTGGTGC |
| | AACACGACGTCAACTTGGGTTGTGTACGGAACCTGCCATCACAAAAAAGGTGAAGCACGGAGATCTA |
| | GAAGAGCTGTGACGCTCCCCTCCCATTCCACTAGAAAGCTGCAAACGCGGTCGCAAACCTGGTTGGA |
| | ATCAAGAGAATACACAAAGCACTTGATTAGAGTCGAAAATTGGATATTCAGGAACCCTGGCTTCGCG |
| | TTAGCAGCAGCTGCCATCGCTTGGCTTTTGGGAAGCTCAACGAGCCAAAAAGTCATATACTTGGTCA |
| | TGATACTGCTGATTGCCCCGGCATACAGCATCAGGTGCATAGGAGTCAGCAATAGGGACTTTGTGGA |
| | AGGTATGTCAGGTGGGACTTGGGTTGATGTTGTCTTGGAACATGGAGGTTGTGTCACCGTAATGGCA |
| | CAGGACAAACCGACTGTCGACATAGAGCTGGTTACAACAACAGTCAGCAACATGGCGGAGGTAAGAT |
| | CCTACTGCTATGAGGCATCAATATCAGACATGGCTTCGGACAGCCGCTGCCCAACACAAGGTGAAGC |
| | CTACCTTGACAAGCAATCAGACACTCAATATGTCTGCAAAAGAACGTTAGTGGACAGAGGCTGGGGA |
| | AATGGATGTGGACTTTTTGGCAAAGGGAGCCTGGTGACATGCGCTAAGTTTGCATGCTCCAAGAAAA |
| | TGACCGGGAAGAGCATCCAGCCAGAGAATCTGGAGTACCGGATAATGCTGTCAGTTCATGGCTCCCA |
| | GCACAGTGGGATGATCGTTAATGACACAGGACATGAAACTGATGAGAATAGAGCGAAGGTTGAGATA |
| | ACGCCCAATTCACCAAGAGCCGAAGCCACCCTGGGGGGTTTTGGAAGCCTAGGACTTGATTGTGAAC |
| | CGAGGACAGGCCTTGACTTTTCAGATTTGTATTACTTGACTATGAATAACAAGCACTGGTTGGTTCA |
| | CAAGGAGTGGTTCCACGACATTCCATTACCTTGGCACGCTGGGGCAGACACCGGAACTCCACACTGG |
| | AACAACAAAGAAGCACTGGTAGAGTTCAAGGACGCACATGCCAAAAGGCAAACTGTCGTGGTTCTAG |
| | GGAGTCAAGAAGGAGCAGTTCACACGGCCCTTGCTGGAGCTCTGGAGGCTGAGATGGATGGTGCAAA |
| | GGGAAGGCTGTCCTCTGGCCACTTGAAATGTCGCCTGAAAATGGATAAACTTAGATTGAAGGGCGTG |
| | TCATACTCCTTGTGTACCGCAGCGTTCACATTCACCAAGATCCCGGCTGAAACACTGCACGGGACAG |
| | TCACAGTGGAGGTACAGTACGCAGGGACAGATGGACCTTGCAAGGTTCCAGCTCAGATGGCGGTGGA |
| | CATGCAAACTCTGACCCCAGTTGGGAGGTTGATAACCGCTAACCCCGTAATCACTGAAAGCACTGAG |
| | AACTCTAAGATGATGCTGGAACTTGATCCACCATTTGGGGACTCTTACATTGTCATAGGAGTCGGGG |
| | AGAAGAAGATCACCCACCACTGGCACAGGAGTGGCAGCACCATTGGAAAAGCATTTGAAGCCACTGT |
| | GAGAGGTGCCAAGAGAATGGCAGTCTTGGGAGACACAGCCTGGGACTTTGGATCAGTTGGAGGCGCT |
| | CTCAACTCATTGGGCAAGGGCATCCATCAAATTTTTGGAGCAGCTTTCAAATCATTGTTTGGAGGAA |
| | TGTCCTGGTTCTCACAAATTCTCATTGGAACGTTGCTGATGTGGTTGGGTCTGAACACAAAGAATGG |
| | ATCTATTTCCCTTATGTGCTTGGCCTTAGGGGGAGTGTTGATCTTCTTATCCACAGCCGTCTCTGCT |
| | GATGTGGGGTGCTCGGTGGACTTCTCAAAGAAGGAGACGAGATGCGGTACAGGGGTGTTCGTCTATA |
| | ACGACGTTGAAGCCTGGAGGGACAGGTACAAGTACCATCCTGACTCCCCCCGTAGATTGGCAGCAGC |
| | AGTCAAGCAAGCCTGGGAAGATGGTATCTGTGGGATCTCCTCTGTTTCAAGAATGGAAAACATCATG |
| | TGGAGATCAGTAGAAGGGGAGCTCAACGCAATCCTGGAAGAGAATGGAGTTCAACTGACGGTCGTTG |
| | TGGGATCTGTAAAAAACCCCATGTGGAGAGGTCCACAGAGATTGCCCGTGCCTGTGAACGAGCTGCC |
| | CCACGGCTGGAAGGCTTGGGGGAAATCGTACTTCGTCAGAGCAGCAAAGACAAATAACAGCTTTGTC |
| | GTGGATGGTGACACACTGAAGGAATGCCCACTCAAACATAGAGCATGGAACAGCTTTCTTGTGGAGG |
| | ATCATGGGTTCGGGGTATTTCACACTAGTGTCTGGCTCAAGGTTAGAGAAGATTATTCATTAGAGTG |
| | TGATCCAGCCGTTATTGGAACAGCTGTTAAGGGAAAGGAGGCTGTACACAGTGATCTAGGCTACTGG |
| | ATTGAGAGTGAGAAGAATGACACATGGAGGCTGAAGAGGGCCCATCTGATCGAGATGAAAACATGTG |
| | AATGGCCAAAGTCCCACACATTGTGGACAGATGGAATAGAAGAGAGTGATCTGATCATACCCAAGTC |
| | TTTAGCTGGGCCACTCAGCCATCACAATACCAGAGAGGGCTACAGGACCCAAATGAAAGGGCCATGG |
| | CACAGTGAAGAGCTTGAAATTCGGTTTGAGGAATGCCCAGGCACTAAGGTCCACGTGGAGGAAACAT |
| | GTGGAACAAGAGGACCATCTCTGAGATCAACCACTGCAAGCGGAAGGGTGATCGAGGAATGGTGCTG |
| | CAGGGAGTGCACAATGCCCCCACTGTCGTTCCGGGCTAAAGATGGCTGTTGGTATGGAATGGAGATA |
| | AGGCCCAGGAAAGAACCAGAAAGTAACTTAGTAAGGTCAATGGTGACTGCAGGATCAACTGATCACA |
| | TGGATCACTTCTCCCTTGGAGTGCTTGTGATTCTGCTCATGGTGCAGGAAGGGCTGAAGAAGAGAAT |
| | GACCACAAAGATCATCATAAGCACATCAATGGCAGTGCTGGTAGCTATGATCCTGGGAGGATTTTCA |
| | ATGAGTGACCTGGCTAAGCTTGCAATTTTGATGGGTGCCACCTTCGCGGAAATGAACACTGGAGGAG |
| | ATGTAGCTCATCTGGCGCTGATAGCGGCATTCAAAGTCAGACCAGCGTTGCTGGTATCTTTCATCTT |
| | CAGAGCTAATTGGACACCCCGTGAAAGCATGCTGCTGGCCTTGGCCTCGTGTCTTTTGCAAACTGCG |
| | ATCTCCGCCTTGGAAGGCGACCTGATGGTTCTCATCAATGGTTTTGCTTTGGCCTGGTTGGCAATAC |
| | GAGCGATGGTTGTTCCACGCACTGATAACATCACCTTGGCAATCCTGGCTGCTCTGACACCACTGGC |
| | CCGGGGCACACTGCTTGTGGCGTGGAGAGCAGGCCTTGCTACTTGCGGGGGGTTTATGCTCCTCTCT |
| | CTGAAGGGAAAAGGCAGTGTGAAGAAGAACTTACCATTTGTCATGGCCCTGGGACTAACCGCTGTGA |
| | GGCTGGTCGACCCCATCAACGTGGTGGGACTGCTGTTGCTCACAAGGAGTGGGAAGCGGAGCTGGCC |
| | CCCTAGCGAAGTACTCACAGCTGTTGGCCTGATATGCGCATTGGCTGGAGGGTTCGCCAAGGCAGAT |
| | ATAGAGATGGCTGGGCCCATGGCCGCGGTCGGTCTGCTAATTGTCAGTTACGTGGTCTCAGGAAAGA |
| | GTGTGGACATGTACATTGAAAGAGCAGGTGACATCACATGGGAAAAAGATGCGGAAGTCACTGGAAA |
| | CAGTCCCCGGCTCGATGTGGCGCTAGATGAGAGTGGTGATTTCTCCCTGGTGGAGGATGACGGTCCC |
| | CCCATGAGAGAGATCATACTCAAGGTGGTCCTGATGACCATCTGTGGCATGAACCCAATAGCCATAC |
| | CCTTTGCAGCTGGAGCGTGGTACGTATACGTGAAGACTGGAAAAAGGAGTGGTGCTCTATGGGATGT |
| | GCCTGCTCCCAAGGAAGTAAAAAAGGGGGAGACCACAGATGGAGTGTACAGAGTAATGACTCGTAGA |
| | CTGCTAGGTTCAACACAAGTTGGAGTGGGAGTTATGCAAGAGGGGGTCTTTCACACTATGTGGCACG |
| | TCACAAAAGGATCCGCGCTGAGAAGCGGTGAAGGGAGACTTGATCCATACTGGGGAGATGTCAAGCA |
| | GGATCTGGTGTCATACTGTGGTCCATGGAAGCTAGATGCCGCCTGGGACGGGCACAGCGAGGTGCAG |
| | CTCTTGGCCGTGCCCCCCGGAGAGAGAGCGAGGAACATCCAGACTCTGCCCGGAATATTTAAGACAA |
| | AGGATGGGGACATTGGAGCGGTTGCGCTGGATTACCCAGCAGGAACTTCAGGATCTCCAATCCTAGA |
| | CAAGTGTGGGAGAGTGATAGGACTTTATGGCAATGGGGTCGTGATCAAAAATGGGAGTTATGTTAGT |
| | GCCATCACCCAAGGGAGGAGGGAGGAAGAGACTCCTGTTGAGTGCTTCGAGCCTTCGATGCTGAAGA |
| | AGAAGCAGCTAACTGTCTTAGACTTGCATCCTGGAGCTGGGAAAACCAGGAGAGTTCTTCCTGAAAT |
| | AGTCCGTGAAGCCATAAAAACAAGACTCCGTACTGTGATCTTAGCTCCAACCAGGGTTGTCGCTGCT |
| | GAAATGGAGGAAGCCCTTAGAGGGCTTCCAGTGCGTTATATGACAACAGCAGTCAATGTCACCCACT |
| | CTGGAACAGAAATCGTCGACTTAATGTGCCATGCCACCTTCACTTCACGTCTACTACAGCCAATCAG |
| | AGTCCCTAACTATAATCTGTATATTATGGATGAGGCCCACTTCACAGATCCCTCAAGTATAGCAGCA |
| | AGAGGATACATTTCAACAAGGGTTGAGATGGGCGAGGCGGCTGCCATCTTCATGACCGCCACGCCAC |
| | CAGGAACCCGTGACGCATTTCCGGACTCCAACTCACCAATTATGGACACCGAAGTGGAAGTCCCAGA |
| | GAGAGCCTGGAGCTCAGGCTTTGATTGGGTGACGGATCATTCTGGAAAAACAGTTTGGTTTGTTCCA |
| | AGCGTGAGGAACGGCAATGAGATCGCAGCTTGTCTGACAAAGGCTGGAAAACGGGTCATACAGCTCA |
| | GCAGAAAGACTTTTGAGACAGAGTTCCAGAAAACAAAACATCAAGAGTGGGACTTTGTCGTGACAAC |
| | TGACATTTCAGAGATGGGCGCCAACTTTAAAGCTGACCGTGTCATAGATTCCAGGAGATGCCTAAAG |
| | CCGGTCATACTTGATGGCGAGAGAGTCATTCTGGCTGGACCCATGCCTGTCACACATGCCAGCGCTG |
| | CCCAGAGGAGGGGGCGCATAGGCAGGAATCCCAACAAACCTGGAGATGAGTATCTGTATGGAGGTGG |
| | GTGCGCAGAGACTGACGAAGACCATGCACACTGGCTTGAAGCAAGAATGCTCCTTGACAATATTTAC |
| | CTCCAAGATGGCCTCATAGCCTCGCTCTATCGACCTGAGGCCGACAAAGTAGCAGCCATTGAGGGAG |
| | AGTTCAAGCTTAGGACGGAGCAAAGGAAGACCTTTGTGGAACTCATGAAAAGAGGAGATCTTCCTGT |
| | TTGGCTGGCCTATCAGGTTGCATCTGCCGGAATAACCTACACAGATAGAAGATGGTGCTTTGATGGC |
| | ACGACCAACAACACCATAATGGAAGACAGTGTGCCGGCAGAGGTGTGGACCAGACACGGAGAGAAAA |
| | GAGTGCTCAAACCGAGGTGGATGGACGCCAGAGTTTGTTCAGATCATGCGGCCCTGAAGTCATTCAA |
| | GGAGTTTGCCGCTGGGAAAAGAGGAGCGGCTTTTGGAGTGATGGAAGCCCTGGGAACACTGCCAGGA |
| | CACATGACAGAGAGATTCCAGGAAGCCATTGACAACCTCGCTGTGCTCATGCGGGCAGAGACTGGAA |
| | GCAGGCCTTACAAAGCCGCGGCGGCCCAATTGCCGGAGACCCTAGAGACCATTATGCTTTTGGGGTT |
| | GCTGGGAACAGTCTCGCTGGGAATCTTTTTCGTCTTGATGAGGAACAAGGGCATAGGGAAGATGGGC |
| | TTTGGAATGGTGACTCTTGGGGCCAGCGCATGGCTCATGTGGCTCTCGGAAATTGAGCCAGCCAGAA |
| | TTGCATGTGTCCTCATTGTTGTGTTCCTATTGCTGGTGGTGCTCATACCTGAGCCAGAAAAGCAAAG |
| | ATCTCCCCAGGACAACCAAATGGCAATCATCATCATGGTAGCAGTAGGTCTTCTGGGCTTGATTACC |
| | GCCAATGAACTCGGATGGTTGGAGAGAACAAAGAGTGACCTAAGCCATCTAATGGGAAGGAGAGAGG |
| | AGGGGGCAACCATAGGATTCTCAATGGACATTGACCTGCGGCCAGCCTCAGCTTGGGCCATCTATGC |
| | TGCCTTGACAACTTTCATTACCCCAGCCGTCCAACATGCAGTGACCACTTCATACAACAACTACTCC |
| | TTAATGGCGATGGCCACGCAAGCTGGAGTGTTGTTTGGTATGGGCAAAGGGATGCCATTCTACGCAT |
| | GGGACTTTGGAGTCCCGCTGCTAATGATAGGTTGCTACTCACAATTAACACCCCTGACCCTAATAGT |
| | GGCCATCATTTTGCTCGTGGCGCACTACATGTACTTGATCCCAGGGCTGCAGGCAGCAGCTGCGCGT |
| | GCTGCCCAGAAGAGAACGGCAGCTGGCATCATGAAGAACCCTGTTGTGGATGGAATAGTGGTGACTG |
| | ACATTGACACAATGACAATTGACCCCCAAGTGGAGAAAAAGATGGGACAGGTGCTACTCATAGCAGT |
| | AGCCGTCTCCAGCGCCATACTGTCGCGGACCGCCTGGGGGTGGGGGGAGGCTGGGGCCCTGATCACA |
| | GCTGCAACTTCCACTTTGTGGGAAGGCTCTCCGAACAAGTACTGGAACTCCTCTACAGCCACTTCAC |
| | TGTGTAACATTTTTAGGGGAAGTTACTTGGCTGGAGCTTCTCTAATCTACACAGTAACAAGAAACGC |
| | TGGCTTGGTCAAGAGACGTGGGGGTGGAACAGGAGAGACCCTGGGAGAGAAATGGAAGGCCCGCTTG |
| | AACCAGATGTCGGCCCTGGAGTTCTACTCCTACAAAAAGTCAGGCATCACCGAGGTGTGCAGAGAAG |
| | AGGCCCGCCGCGCCCTCAAGGACGGTGTGGCAACGGGAGGCCATGCTGTGTCCCGAGGAAGTGCAAA |
| | GCTGAGATGGTTGGTGGAGCGGGGATACCTGCAGCCCTATGGAAAGGTCATTGATCTTGGATGTGGC |
| | AGAGGGGGCTGGAGTTACTACGCCGCCACCATCCGCAAAGTTCAAGAAGTGAAAGGATACACAAAAG |
| | GAGGCCCTGGTCATGAAGAACCCATGTTGGTGCAAAGCTATGGGTGGAACATAGTCCGTCTTAAGAG |
| | TGGGGTGGACGTCTTTCATATGGCGGCTGAGCCGTGTGACACGTTGCTGTGTGACATAGGTGAGTCA |
| | TCATCTAGTCCTGAAGTGGAAGAAGCACGGACGCTCAGAGTCCTCTCCATGGTGGGGGATTGGCTTG |
| | AAAAAAGACCAGGAGCCTTTTGTATAAAAGTGTTGTGCCCATACACCAGCACTATGATGGAAACCCT |
| | GGAGCGACTGCAGCGTAGGTATGGGGGAGGACTGGTCAGAGTGCCACTCTCCCGCAACTCTACACAT |
| | GAGATGTACTGGGTCTCTGGAGCGAAAAGCAACACCATAAAAAGTGTGTCCACCACGAGCCAGCTCC |
| | TCTTGGGGCGCATGGACGGGCCCAGGAGGCCAGTGAAATATGAGGAGGATGTGAATCTCGGCTCTGG |
| | CACGCGGGCTGTGGTAAGCTGCGCTGAAGCTCCCAACATGAAGATCATTGGTAACCGCATTGAAAGG |
| | ATCCGCAGTGAGCACGCGGAAACGTGGTTCTTTGACGAGAACCACCCATATAGGACATGGGCTTACC |
| | ATGGAAGCTATGAGGCCCCCACACAAGGGTCAGCGTCCTCTCTAATAAACGGGGTTGTCAGGCTCCT |
| | GTCAAAACCCTGGGATGTGGTGACTGGAGTCACAGGAATAGCCATGACCGACACCACACCGTATGGT |
| | CAGCAAAGAGTTTTCAAGGAAAAAGTGGACACTAGGGTGCCAGACCCCCAAGAAGGCACTCGTCAGG |
| | TTATGAGCATGGTCTCTTCCTGGTTGTGGAAAGAGCTAGGCAAACACAAACGGCCACGAGTCTGTAC |
| | CAAAGAAGAGTTCATCAACAAGGTTCGTAGCAATGCAGCATTAGGGGCAATATTTGAAGAGGAAAAA |
| | GAGTGGAAGACTGCAGTGGAAGCTGTGAAGGATCCAAGGTTCTGGGCTCTAGTGGACAAGGAAAGAG |
| | AGCACCACCTGAGAGGAGAGTGCCAGAGTTGTGTGTACAACATGATGGGAAAAAGAGAAAAGAAACA |
| | AGGGGAATTTGGAAAGGCCAAGGGCAGCCGCGCCATCTGGTATATGTGGCTAGGGGCTAGATTTCTA |
| | GAGTTCGAAGCCCTTGGATTCTTGAACGAGGATCACTGGATGGGGAGAGAGAACTCAGGAGGTGGTG |
| | TTGAAGGGCTGGGATTACAAAGACTCGGATATGTCCTAGAAGAGATGAGTCGCATACCAGGAGGAAG |
| | GATGTATGCAGATGACACTGCTGGCTGGGACACCCGCATCAGCAGGTTTGATCTGGAGAATGAAGCT |
| | CTAATCACCAACCAAATGGAGAAAGGGCACAGGGCCTTGGCATTGGCCATAATCAAGTACACATACC |
| | AAAACAAAGTGGTAAAGGTCCTTAGACCAGCTGAAAAAGGGAAGACAGTTATGGACATTATTTCGAG |
| | ACAAGACCAAAGGGGGAGCGGACAAGTTGTCACTTACGCTCTTAACACATTTACCAACCTAGTGGTG |
| | CAACTCATTCGGAATATGGAGGCTGAGGAAGTTCTAGAGATGCAAGACTTGTGGCTGCTGCGGAGGT |
| | CAGAGAAAGTGACCAACTGGTTGCAGAGCAACGGATGGGATAGGCTCAAACGAATGGCAGTCAGTGG |
| | AGATGATTGCGTTGTGAAGCCAATTGATGATAGGTTTGCACATGCCCTCAGGTTCTTGAATGATATG |
| | GGAAAAGTTAGGAAGGACACACAAGAGTGGAAACCCTCAACTGGATGGGACAACTGGGAAGAAGTTC |
| | CGTTTTGCTCCCACCACTTCAACAAGCTCCATCTCAAGGACGGGAGGTCCATTGTGGTTCCCTGCCG |
| | CCACCAAGATGAACTGATTGGCCGGGCCCGCGTCTCTCCAGGGGCGGGATGGAGCATCCGGGAGACT |
| | GCTTGCCTAGCAAAATCATATGCGCAAATGTGGCAGCTCCTTTATTTCCACAGAAGGGACCTCCGAC |
| | TGATGGCCAATGCCATTTGTTCATCTGTGCCAGTTGACTGGGTTCCAACTGGGAGAACTACCTGGTC |
| | AATCCATGGAAAGGGAGAATGGATGACCACTGAAGACATGCTTGTGGTGTGGAACAGAGTGTGGATT |
| | GAGGAGAACGACCACATGGAAGACAAGACCCCAGTTACGAAATGGACAGACATTCCCTATTTGGGAA |
| | AAAGGGAAGACTTGTGGTGTGGATCTCTCATAGGGCACAGACCGCGCACCACCTGGGCTGAGAACAT |
| | TAAAAACACAGTCAACATGGTGCGCAGGATCATAGGTGATGAAGAAAAGTACATGGACTACCTATCC |
| | ACCCAAGTTCGCTACTTGGGTGAAGAAGGGTCTACACCTGGAGTGCTGTAAGCACCAATCTTAGTGT |
| | TGTCAGGCCTGCTAGTCAGCCACAGCTTGGGGAAAGCTGTGCAGCCTGTGACCCCCCCAGGAGAAGC |
| | TGGGAAACCAAGCCTATAGTCAGGCCGAGAACGCCATGGCACGGAAGAAGCCATGCTGCCTGTGAGC |
| | CCCTCAGAGGACACTGAGTCAAAAAACCCCACGCGCTTGGAGGCGCAGGATGGGAAAAGAAGGTGGC |
| | GACCTTCCCCACCCTTCAATCTGGGGCCTGAACTGGAGATCAGCTGTGGATCTCCAGAAGAGGGACT |
| | AGTGGTTAGAGGAGGTCCACAGAAGGGACCTCCGCAGCATATTGACGCTGGGAAAGACCAGAGACTC |
| | CATGAGTTTCCACCACGCTGGCCGCCAGGCACAGATCGCCGATTAAGAATTCCACCAAAAAGAATTC |
| | TGCTGCCATACCATCTCCGAGGATGTCCCATAAACTTGGTTGCAGAGTTTTGTGAAATGTTGCACTC |
| | CAGTAGAAGGCAGCAGACTAGGCTGGGCGTGGCGGCTCATGCCTGTAATCCCAGCACTTTGGGAGGT |
| | TGAGGCAGGTGGATCACCTGAGGTCAGGAGTTCGAGACCAGCTTGGCCAACATGGTGAAACCCCGTC |
| | TCTACTAGAAACACAAACAATTAGCCAGGCATGGTGGCGGGCGCTGTAATCCTAGCTACTCGGGAGA |
| | CTGAGGCAGGAGAATTGCTTGAACCCAGGAGGCAGAGGTTGCAGTGAGCTGAGATCGCACCATTGCA |
| | CTCCAGCCTGGGCGACAGAGCAAGACTCCGTCT |
| 295 | GenBank Accession Nos. NC_035889.1/ KU527068.1 (Zika virus isolate ZIKV/H. |
| | sapiens/Brazil/Natal/2015, complete genome) |
| | AGTTGTTGATCTGTGTGAATCAGACTGCGACAGTTCGAGTTTGAAGCGAAAGCTAGCAACAGTATCA |
| | ACAGGTTTTATTTTGGATTTGGAAACGAGAGTTTCTGGTCATGAAAAACCCAAAAAAGAAATCCGGA |
| | GGATTCCGGATTGTCAATATGCTAAAACGCGGAGTAGCCCGTGTGAGCCCCTTTGGGGGCTTGAAGA |
| | GGCTGCCAGCCGGACTTCTGCTGGGTCATGGGCCCATCAGGATGGTCTTGGCAATTCTAGCCTTTTT |
| | GAGATTCACGGCAATCAAGCCATCACTGGGTCTCATCAATAGATGGGGTTCAGTGGGGAAAAAAGAG |
| | GCTATGGAAATAATAAAGAAGTTCAAGAAAGATCTGGCTGCCATGCTGAGAATAATCAATGCTAGGA |
| | AGGAGAAGAAGAGACGAGGCGCAGATACTAGTGTCGGAATTGTTGGCCTCCTGCTGACCACAGCTAT |
| | GGCAGCGGAGGTCACTAGACGTGGGAGTGCATACTATATGTACTTGGACAGAAACGATGCTGGGGAG |
| | GCCATATCTTTTCCAACCACATTGGGGATGAATAAGTGTTATATACAGATCATGGATCTTGGACACA |
| | TGTGTGATGCCACCATGAGCTATGAATGCCCTATGCTGGATGAGGGGGTGGAACCAGATGACGTCGA |
| | TTGTTGGTGCAACACGACGTCAACTTGGGTTGTGTACGGAACCTGCCATCACAAAAAAGGTGAAGCA |
| | CGGAGATCTAGAAGAGCTGTGACGCTCCCCTCCCATTCCACTAGGAAGCTGCAAACGCGGTCGCAAA |
| | CCTGGTTGGAATCAAGAGAATACACAAAGCACTTGATTAGAGTCGAAAATTGGATATTCAGGAACCC |
| | TGGCTTCGCGTTAGCAGCAGCTGCCATCGCTTGGCTTTTGGGAAGCTCAACGAGCCAAAAAGTCATA |
| | TACTTGGTCATGATACTGCTGATTGCCCCGGCATACAGCATCAGGTGCATAGGAGTCAGCAATAGGG |
| | ACTTTGTGGAAGGTATGTCAGGTGGGACTTGGGTTGATGTTGTCTTGGAACATGGAGGTTGTGTCAC |
| | CGTAATGGCACAGGACAAACCGACTGTCGACATAGAGCTGGTTACAACAACAGTCAGCAACATGGCG |
| | GAGGTAAGATCCTACTGCTATGAGGCATCAATATCAGACATGGCTTCGGACAGCCGCTGCCCAACAC |
| | AAGGTGAAGCCTACCTTGACAAGCAATCAGACACTCAATATGTCTGCAAAAGAACGTTAGTGGACAG |
| | AGGCTGGGGAAATGGATGTGGACTTTTTGGCAAAGGGAGCCTGGTGACATGCGCTAAGTTTGCATGC |
| | TCCAAGAAAATGACCGGGAAGAGCATCCAGCCAGAGAATCTGGAGTACCGGATAATGCTGTCAGTTC |
| | ATGGCTCCCAGCACAGTGGGATGATCGTTAATGACACAGGACATGAAACTGATGAGAATAGAGCGAA |
| | GGTTGAGATAACGCCCAATTCACCAAGAGCCGAAGCCACCCTGGGGGGTTTTGGAAGCCTAGGACTT |
| | GATTGTGAACCGAGGACAGGCCTTGACTTTTCAGATTTGTATTACTTGACTATGAATAACAAGCACT |
| | GGTTGGTCCACAAGGAGTGGTTCCACGACATTCCATTACCTTGGCACGCTGGGGCAGACACCGGAAC |
| | TCCACACTGGAACAACAAAGAAGCACTGGTAGAGTTCAAGGACGCACATGCCAAAAGGCAAACTGTC |
| | GTGGTTCTAGGGAGTCAAGAAGGAGCAGTTCACACGGCCCTTGCTGGAGCTCTGGAGGCTGAGATGG |
| | ATGGTGCAAAGGGAAGGCTGTCCTCTGGCCACTTGAAATGTCGCCTGAAAATGGATAAACTTAGATT |
| | GAAGGGCGTGTCATACTCCTTGTGTACCGCAGCGTTCACATTCACCAAGATCCCGGCTGAAACACTG |
| | CACGGGACAGTCACAGTGGAGGTACAGTACGCAGGGACAGATGGACCTTGCAAGGTTCCAGCTCAGA |
| | TGGCGGTGGACATGCAAACTCTGACCCCAGTTGGGAGGTTGATAACCGCTAACCCCGTAATCACTGA |
| | AAGCACTGAGAACTCTAAGATGATGCTGGAACTTGATCCACCATTTGGGGACTCTTACATTGTCATA |
| | GGAGTCGGGGAGAAGAAGATCACCCACCACTGGCACAGGAGTGGCAGCACCATTGGAAAAGCATTTG |
| | AAGCCACTGTGAGAGGTGCCAAGAGAATGGCAGTCTTGGGAGACACAGCCTGGGACTTTGGATCAGT |
| | TGGAGGCGCTCTCAACTCATTGGGCAAGGGCATCCATCAAATTTTTGGAGCAGCTTTCAAATCATTG |
| | TTTGGAGGAATGTCCTGGTTCTCACAAATCCTCATTGGAACGTTGCTGATGTGGTTGGGTCTGAACA |
| | CAAAGAATGGATCTATTTCCCTTATGTGCTTGGCCTTAGGGGGAGTGTTGATCTTCTTATCCACAGC |
| | CGTCTCTGCTGATGTGGGGTGCTCGGTGGACTTCTCAAAGAAGGAGACGAGATGCGGTACAGGGGTG |
| | TTCGTCTATAACGACGTTGAAGCCTGGAGGGACAGGTACAAGTACCATCCTGACTCCCCCCGTAGAT |
| | TGGCAGCAGCAGTCAAGCAAGCCTGGGAAGATGGTATCTGCGGGATCTCCTCTGTTTCAAGAATGGA |
| | GAACATCATGTGGAGATCAGTAGAAGGGGAGCTCAACGCAATCTTGGAAGAGAATGGAGTTCAACTG |
| | ACGGTCGTTGTGGGATCTGTAAAAAACCCCATGTGGAGAGGTCCACAGAGATTGCCCGTGCCTGTGA |
| | ACGAGCTGCCCCACGGCTGGAAGGCTTGGGGGAAATCGTACTTCGTCAGAGCAGCAAAGACAAATAA |
| | CAGCTTTGTCGTGGATGGTGACACACTGAAGGAATGCCCACTCGAACATAGAGCATGGAACAGCTTT |
| | CTTGTGGAGGATCATGGGTTCGGGGTATTTCACACTAGTGTCTGGCTCAAGGTTAGAGAAGATTATT |
| | CATTAGAGTGTGATCCAGCCGTTATTGGAACAGCTGTTAAGGGGAAGGAGGCTGTACACAGTGATCT |
| | AGGCTACTGGATTGAGAGTGAGAAGAATGACACATGGAGGCTGAAGAGGGCCCATCTAATCGAGATG |
| | AAAACATGTGAATGGCCAAAGTCCCACACATTGTGGGCAGATGGAATAGAAGAGAGTGATCTGATCA |
| | TTCCCAAGTCTTTAGCTGGGCCACTCAGCCATCACAATACCAGAGAGGGCTACAGGACCCAAATGAA |
| | AGGGCCATGGCACAGTGAAGAGCTTGAAATTCGGTTTGAGGAATGCCCGGGCACTAAGGTCCACGTG |
| | GAGGAAACATGTGGAACAAGAGGACCATCTCTGAGATCAACCACTGCAAGCGGAAGGGTGATCGAGG |
| | AATGGTGCTGCAGGGAGTGCACAATGCCCCCACTGTCGTTCCGGGCTAAAGATGGCTGTTGGTATGG |
| | AATGGAGATAAGGCCCAGGAAAGAACCAGAAAGCAACTTAGTAAGGTCAGTGGTGACTGCAGGATCA |
| | ACTGATCACATGGATCACTTCTCCCTTGGAGTGCTTGTGATTCTGCTCATGGTGCAGGAAGGGCTGA |
| | AGAAGAGAATGACCACAAAGATCATCATAAGCACATCAATGGCAGTGCTGGTAGCTATGATCCTGGG |
| | AGGATTTTCAATGAGTGACCTGGCTAAGCTTGCAATTTTGATGGGCGCCACCTTCGCGGAAATGAAC |
| | ACTGGAGGAGATGTAGCTCATCTGGCGCTGATAGCGGCATTCAAAGTCAGACCAGCGTTGCTGGTAT |
| | CTTTCATCTTCAGAGCTAATTGGACACCCCGTGAAAGCATGCTGCTGGCCTTGGCCTCGTGTCTTTT |
| | GCAAACTGCGATCTCCGCCTTGGAAGGCGACCTGATGGTTCTCATCAATGGTTTTGCTTTGGCCTGG |
| | TTGGCAATACGAGCGATGGTTGTTCCACGCACTGATAACATCACCTTGGCAATCCTGGCTGCTCTGA |
| | CACCACTGGCCCGGGGCACACTGCTTGTGGCGTGGAGAGCAGGCCTTGCTACTTGCGGGGGGTTTAT |
| | GCTCCTCTCTCTGAAGGGAAAAGGCAGTGTGAAGAAGAACTTACCATTTGTCATGGCCCTGGGACTA |
| | ACCGCTGTGAGGCTGGTCGACCCCATCAACGTGGTGGGACTGCTGTTGCTCACAAGGAGTGGGAAGC |
| | GGAGCTGGCCCCCTAGCGAAGTACTCACAGCTGTTGGCCTGATATGCGCATTGGCTGGAGGGTTCGC |
| | CAAGGCAGATATAGAGATGGCTGGGCCCATGGCCGCGGTCGGTCTGCTAATTGTCAGTTACGTGGTC |
| | TCAGGAAAGAGTGTGGAGATGTACATTGAAAGAGCAGGTGACATCACATGGGAAAAAGATGCGGAAG |
| | TCACTGGAAACAGTCCCCGGCTCGATGTGGCGCTAGATGAGAGTGGTGATTTCTCCCTGGTGGAGGA |
| | TGACGGTCCCCCCATGAGAGAGATCATACTCAAGGTGGTCCTGATGACCATCTGTGGCATGAACCCA |
| | ATAGCCATACCCTTTGCAGCTGGAGCGTGGTACGTATACGTGAAGACTGGAAAAAGGAGTGGTGCTC |
| | TATGGGATGTGCCTGCTCCCAAGGAAGTAAAAAAGGGGGAGACCACAGATGGAGTGTACAGAGTAAT |
| | GACTCGTAGACTGCTAGGTTCAACACAAGTTGGAGTGGGAGTTATGCAAGAGGGGGTCTTTCACACT |
| | ATGTGGCACGTCACAAAAGGATCCGCGCTGAGAAGCGGTGAAGGGAGACTTGATCCATACTGGGGAG |
| | ATGTCAAGCAGGATCTGGTGTCATACTGTGGTCCATGGAAGCTAGATGCCGCCTGGGACGGGCACAG |
| | CGAGGTGCAGCTCTTGGCCGTGCCCCCCGGAGAGAGAGCGAGGAACATCCAGACTCTGCCCGGAATA |
| | TTTAAGACAAAGGATGGGGACATTGGAGCGGTTGCGCTGGATTACCCAGCAGGAACTTCAGGATCTC |
| | CAATCCTAGACAAGTGTGGGAGAGTGATAGGACTTTATGGCAATGGGGTCGTGATCAAAAATGGGAG |
| | TTATGTTAGTGCCATCACCCAAGGGAGGAGGGAGGAAGAGACTCCTGTTGAGTGCTTCGAGCCTTCG |
| | ATGCTGAAGAAGAAGCAGCTAACTGTCTTAGACTTGCATCCTGGAGCTGGGAAAACCAGGAGAGTTC |
| | TTCCTGAAATAGTCCGTGAAGCCATAAAAACAAGACTCCGTACTGTGATCTTAGCTCCAACCAGGGT |
| | TGTCGCTGCTGAAATGGAGGAAGCCCTTAGAGGGCTTCCAGTGCGTTATATGACAACAGCAGTCAAT |
| | GTCACCCACTCTGGAACAGAAATCGTCGACTTAATGTGCCATGCCACCTTCACTTCACGTCTACTAC |
| | AGCCAATCAGAGTCCCCAACTATAATCTGTATATTATGGATGAGGCCCACTTCACAGATCCCTCAAG |
| | TATAGCAGCAAGAGGATACATTTCAACAAGGGTTGAGATGGGCGAGGCGGCTGCCATCTTCATGACC |
| | GCCACGCCACCAGGAACCCGTGACGCATTTCCGGACTCCAACTCACCAATTATGGACACCGAAGTGG |
| | AAGTCCCAGAGAGAGCCTGGAGCTCAGGCTTTGATTGGGTGACGGATCATTCTGGAAAAACAGTTTG |
| | GTTTGTTCCAAGCGTGAGGAACGGCAATGAGATCGCAGCTTGTCTGACAAAGGCTGGAAAACGGGTC |
| | ATACAGCTCAGCAGAAAGACTTTTGAGACAGAGTTCCAGAAAACAAAACATCAAGAGTGGGACTTTG |
| | TCGTGACAACTGACATTTCAGAGATGGGCGCCAACTTTAAAGCTGACCGTGTCATAGATTCCAGGAG |
| | ATGCCTAAAGCCGGTCATACTTGATGGCGAGAGAGTCATTTTGGCTGGACCCATGCCTGTCACACAT |
| | GCCAGCGCTGCCCAGAGGAGGGGGCGCATAGGCAGGAATCCCAACAAACCTGGAGATGAGTATCTGT |
| | ATGGAGGTGGGTGCGCAGAGACTGACGAAGACCATGCACACTGGCTTGAAGCAAGAATGCTCCTTGA |
| | CAATATTTACCTCCAAGATGGCCTCATAGCCTCGCTCTATCGACCTGAGGCCGACAAAGTAGCAGCC |
| | ATTGAGGGAGAGTTCAAGCTTAGGACGGAGCAAAGGAAGACCTTTGTGGAACTCATGAAAAGAGGAG |
| | ATCTTCCTGTTTGGCTGGCCTATCAGGTTGCATCTGCCGGAATAACCTACACAGATAGAAGATGGTG |
| | CTTTGATGGCACGACCAACAACACCATAATGGAAGACAGTGTGCCGGCAGAGGTGTGGACCAGACAC |
| | GGAGAGAAAAGAGTGCTCAAACCGAGGTGGATGGACGCCAGAGTTTGTTCAGATCATGCGGCCCTGA |
| | AGTCATTCAAGGAGTTTGCCGCTGGGAAAAGAGGAGCGGCTTTTGGAGTGATGGAAGCCCTGGGAAC |
| | ACTGCCAGGACACATGACAGAGAGATTCCAGGAAGCCATTGACAACCTCGCTGTGCTCATGCGGGCA |
| | GAGACTGGAAGCAGGCCTTACAAAGCCGCGGCGGCCCAATTGCCGGAGACCCTAGAGACCATTATGC |
| | TTTTGGGGTTGCTGGGAACAGTCTCGCTGGGAATCTTTTTCGTCTTGATGAGGAACAAGGGCATAGG |
| | GAAGATGGGCTTTGGAATGGTGACTCTTGGGGCCAGCGCATGGCTCATGTGGCTCTCGGAAATTGAG |
| | CCAGCCAGAATTGCATGTGTCCTCATTGTTGTGTTCCTATTGCTGGTGGTGCTCATACCTGAGCCAG |
| | AAAAGCAAAGATCTCCCCAGGACAACCAAATGGCAATCATCATCATGGTAGCAGTAGGTCTTCTGGG |
| | CTTGATTACCGCCAATGAACTCGGATGGTTGGAGAGAACAAAGAGTGACCTAAGCCATCTAATGGGA |
| | AGGAGAGAGGAGGGAGCAACCATAGGATTCTCAATGGACATTGACCTGCGGCCAGCCTCAGCTTGGG |
| | CCATCTATGCTGCCTTGACAACTTTCATTACCCCAGCCGTCCAACATGCAGTGACCACTTCATACAA |
| | CAACTACTCCTTAATGGCGATGGCCACGCAAGCTGGAGTGTTGTTTGGTATGGGCAAAGGGATGCCA |
| | TTCTACGCATGGGACTTTGGAGTCCCGCTGCTAATGATAGGTTGCTACTCACAATTAACACCCCTGA |
| | CCCTAATAGTGGCCATCATTTTGCTCGTGGCGCACTACATGTACTTGATCCCAGGGCTGCAGGCAGC |
| | AGCTGCGCGTGCTGCCCAGAAGAGAACGGCAGCTGGCATCATGAAGAACCCTGTTGTGGATGGAATA |
| | GTGGTGACTGACATTGACACAATGACAATTGACCCCCAAGTGGAGAAAAAGATGGGACAGGTGCTAC |
| | TCATAGCAGTAGCAGTCTCCAGCGCCATACTGTCGCGGACCGCCTGGGGGTGGGGGGAGGCTGGGGC |
| | CCTGATCACAGCCGCAACTTCCACTTTGTGGGAAGGCTCTCCGAACAAGTACTGGAACTCCTCTACA |
| | GCCACTTCACTGTGTAACATTTTTAGGGGAAGTTACTTGGCTGGAGCTTCTCTAATCTACATAGTAA |
| | CAAGAAACGCTGGCTTGGTCAAGAGACGTGGGGGTGGAACAGGAGAGACCCTGGGAGAGAAATGGAA |
| | GGCCCGCTTGAACCAGATGTCGGCCCTGGAGTTCTACTCCTACAAAAAGTCAGGCATCACCGAGGTG |
| | TGCAGAGAAGAGGCCCGCCGCGCCCTCAAGGATGGTGTGGCAACGGGAGGCCATGCTGTGTCCCGAG |
| | GAAGTGCAAAGCTGAGATGGTTGGTGGAGCGGGGATACCTGCAGCCCTATGGAAAGGTCATTGATCT |
| | TGGATGTGGCAGAGGGGGCTGGAGTTACTACGCCGCCACCATCCGCAAAGTTCAAGAAGTGAAAGGA |
| | TACACAAAAGGAGGCCCTGGTCATGAAGAACCCGTGTTGGTGCAAAGCTATGGGTGGAACATAGTCC |
| | GTCTTAAGAGTGGGGTGGACGTCTTTCATATGGCGGCTGAGCCGTGTGACACGTTGCTGTGTGACAT |
| | AGGTGAGTCATCATCTAGTCCTGAAGTGGAAGAAGCACGGACGCTCAGAGTCCTCTCCATGGTGGGG |
| | GATTGGCTTGAAAAAAGACCAGGAGCCTTTTGTATAAAAGTGTTGTGCCCATACACCAGCACTATGA |
| | TGGAAACCCTGGAGCGACTGCAGCGTAGGTATGGGGGAGGACTGGTCAGAGTGCCACTCTCCCGCAA |
| | CTCTACACATGAGATGTACTGGGTCTCTGGAGCGAAAAGCAACACCATAAAAAGTGTGTCCACCACG |
| | AGCCAGCTCCTCTTGGGGCGCATGGACGGGCCTAGGAGGCCAGTGAAATATGAGGAGGATGTGAATC |
| | TCGGCTCTGGCACGCGGGCTGTGGTAAGCTGCGCTGAAGCTCCCAACATGAAGATCATTGGTAACCG |
| | CATTGAAAGGATCCGCAGTGAGCACGCGGAAACGTGGTTCTTTGACGAGAACCACCCATATAGGACA |
| | TGGGCTTACCATGGAAGCTATGAGGCCCCCACACAAGGGTCAGCGTCCTCTCTAATAAACGGGGTTG |
| | TCAGGCTCCTGTCAAAACCCTGGGATGTGGTGACTGGAGTCACAGGAATAGCCATGACCGACACCAC |
| | ACCGTATGGTCAGCAAAGAGTTTTCAAGGAAAAAGTGGACACTAGGGTGCCAGACCCCCAAGAAGGC |
| | ACTCGTCAGGTTATGAGCATGGTCTCTTCCTGGTTGTGGAAAGAGCTAGGCAAACACAAACGGCCAC |
| | GAGTCTGTACCAAAGAAGAGTTCATCAACAAGGTTCGTAGCAATGCAGCATTAGGGGCAATATTTGA |
| | AGAGGAAAAAGAGTGGAAGACTGCAGTGGAAGCTGTGAACGATCCAAGGTTCTGGGCTCTAGTGGAC |
| | AAGGAAAGAGAGCACCACCTGAGAGGAGAGTGCCAGAGTTGTGTGTACAACATGATGGGAAAAAGAG |
| | AAAAGAAACAAGGGGAATTTGGAAAGGCCAAGGGCAGCCGCGCCATCTGGTATATGTGGCTAGGGGC |
| | TAGATTTCTAGAGTTCGAAGCCCTTGGATTCTTGAACGAGGATCACTGGATGGGGAGAGAGAACTCA |
| | GGAGGTGGTGTTGAAGGGCTGGGATTACAAAGACTCGGATATGTCCTAGAAGAGATGAGTCGCATAC |
| | CAGGAGGAAGGATGTATGCAGATGACACTGCTGGCTGGGACACCCGCATCAGCAGGTTCGATCTGGA |
| | GAATGAAGCTCTAATCACCAACCAAATGGAGAAAGGGCATAGGGCCTTGGCATTGGCCATAATCAAG |
| | TACACATACCAAAACAAAGTGGTAAAGGTCCTTAGACCAGCTGAAAAAGGGAAAACAGTTATGGACA |
| | TTATTTCGAGACAAGACCAAAGGGGGAGCGGACAAGTTGTCACTTACGCTCTTAACACATTTACCAA |
| | CCTAGTGGTGCAACTCATTCGGAATATGGAGGCTGAGGAAGTTCTAGAGATGCAAGACTTGTGGCTG |
| | CTGCGGAGGTCAGAGAAAGTGACCAACTGGTTGCAGAGCAACGGATGGGATAGGCTCAAACGAATGG |
| | CAGTCAGTGGAGATGATTGCGTTGTGAAGCCAATTGATGATAGGTTTGCACATGCCCTCAGGTTCTT |
| | GAATGATATGGGAAAAGTTAGGAAGGACACACAAGAGTGGAAACCCTCAACTGGATGGGACAACTGG |
| | GAAGAAGTTCCGTTTTGCTCCCACCACTTCAACAAGCTCCATCTCAAGGACGGGAGGTCCATTGTGG |
| | TTCCCTGCCGCCACCAAGATGAACTGATTGGCCGGGCCCGCGTCTCTCCAGGGGCGGGATGGAGCAT |
| | CCGGGAGACTGCTTGCCTAGCAAAATCATATGCGCAAATGTGGCAGCTCCTTTATTTCCACAGAAGG |
| | GACCTCCGACTGATGGCCAATGCCATTTGTTCATCTGTGCCAGTTGACTGGGTTCCAACTGGGAGAA |
| | CTACCTGGTCAATCCATGGAAAGGGAGAATGGATGACCACTGAAGACATGCTTGTGGTGTGGAACAG |
| | AGTGTGGATTGAGGAGAACGACCACATGGAAGACAAGACCCCAGTTACGAAATGGACAGACATTCCC |
| | TATTTGGGAAAAAGGGAAGACTTGTGGTGTGGATCTCTCATAGGGCACAGACCGCGCACCACCTGGG |
| | GTGAGAACATTAAAAACACAGTCAACATGGTGCGCAGGATCATAGGTGATGAAGAAAAGTACATGGA |
| | CTACCTATCCACCCAAGTTCGCTACTTGGGTGAAGAAGGGTCTACACCTGGAGTGCTGTAAGCACCA |
| | ATCTTAATGTTGTCAGGCCTGCTAGTCAGCCACAGCTTGGGGAAAGCTGTGCAGCCTGTGACCCCCC |
| | CAGGAGAAGCTGGGAAACCAAGCCTATAGTCAGGCCGAGAACGCCATGGCACGGAAGAAGCCATGCT |
| | GCCTGTGAGCCCCTCAGAGGACACTGAGTCAAAAAACCCCATGCGCTTGGAGGCGCAGGATGGGAAA |
| | AGAAGGTGGCGACCTTCCCCACCCTTCAATCTGGGGCCTGAACTGGAGATCAGCTGTGGATCTCCAG |
| | AAGAGGGACTAGTGGTTAGAGGAGACCCCCCGGAAAACGCAAAACAGCATATTGACGCTGGGAAAGA |
| | CCAGAGACTCCATGAGTTTCCACCACGCTGGCCGCCAGGCACAGATCGCCGAATAGCGGCGGCCGGT |
| | GTGGGGAAATCCATGGGTCTT |
| 300 | GenBank Accession No. AB047639.1 (Hepatitis C virus (isolate JFH-1) genomic |
| | RNA, complete genome) |
| | ACCTGCCCCTAATAGGGGCGACACTCCGCCATGAATCACTCCCCTGTGAGGAACTACTGTCTTCACG |
| | CAGAAAGCGCCTAGCCATGGCGTTAGTATGAGTGTCGTACAGCCTCCAGGCCCCCCCCTCCCGGGAG |
| | AGCCATAGTGGTCTGCGGAACCGGTGAGTACACCGGAATTGCCGGGAAGACTGGGTCCTTTCTTGGA |
| | TAAACCCACTCTATGCCCGGCCATTTGGGCGTGCCCCCGCAAGACTGCTAGCCGAGTAGCGTTGGGT |
| | TGCGAAAGGCCTTGTGGTACTGCCTGATAGGGCGCTTGCGAGTGCCCCGGGAGGTCTCGTAGACCGT |
| | GCACCATGAGCACAAATCCTAAACCTCAAAGAAAAACCAAAAGAAACACCAACCGTCGCCCAGAAGA |
| | CGTTAAGTTCCCGGGCGGCGGCCAGATCGTTGGCGGAGTATACTTGTTGCCGCGCAGGGGCCCCAGG |
| | TTGGGTGTGCGCACGACAAGGAAAACTTCGGAGCGGTCCCAGCCACGTGGGAGACGCCAGCCCATCC |
| | CCAAAGATCGGCGCTCCACTGGCAAGGCCTGGGGAAAACCAGGTCGCCCCTGGCCCCTATATGGGAA |
| | TGAGGGACTCGGCTGGGCAGGATGGCTCCTGTCCCCCCGAGGCTCTCGCCCCTCCTGGGGCCCCACT |
| | GACCCCCGGCATAGGTCGCGCAACGTGGGTAAAGTCATCGACACCCTAACGTGTGGCTTTGCCGACC |
| | TCATGGGGTACATCCCCGTCGTAGGCGCCCCGCTTAGTGGCGCCGCCAGAGCTGTCGCGCACGGCGT |
| | GAGAGTCCTGGAGGACGGGGTTAATTATGCAACAGGGAACCTACCCGGTTTCCCCTTTTCTATCTTC |
| | TTGCTGGCCCTGTTGTCCTGCATCACCGTTCCGGTCTCTGCTGCCCAGGTGAAGAATACCAGTAGCA |
| | GCTACATGGTGACCAATGACTGCTCCAATGACAGCATCACTTGGCAGCTCGAGGCTGCGGTTCTCCA |
| | CGTCCCCGGGTGCGTCCCGTGCGAGAGAGTGGGGAATACGTCACGGTGTTGGGTGCCAGTCTCGCCA |
| | AACATGGCTGTGCGGCAGCCCGGTGCCCTCACGCAGGGTCTGCGGACGCACATCGATATGGTTGTGA |
| | TGTCCGCCACCTTCTGCTCTGCTCTCTACGTGGGGGACCTCTGTGGCGGGGTGATGCTCGCGGCCCA |
| | GGTGTTCATCGTCTCGCCGCAGTACCACTGGTTTGTGCAAGAATGCAATTGCTCCATCTACCCTGGC |
| | ACCATCACTGGACACCGCATGGCATGGGACATGATGATGAACTGGTCGCCCACGGCCACCATGATCC |
| | TGGCGTACGTGATGCGCGTCCCCGAGGTCATCATAGACATCGTTAGCGGGGCTCACTGGGGCGTCAT |
| | GTTCGGCTTGGCCTACTTCTCTATGCAGGGAGCGTGGGCGAAGGTCATTGTCATCCTTCTGCTGGCC |
| | GCTGGGGTGGACGCGGGCACCACCACCGTTGGAGGCGCTGTTGCACGTTCCACCAACGTGATTGCCG |
| | GCGTGTTCAGCCATGGCCCTCAGCAGAACATTCAGCTCATTAACACCAACGGCAGTTGGCACATCAA |
| | CCGTACTGCCTTGAATTGCAATGACTCCTTGAACACCGGCTTTCTCGCGGCCTTGTTCTACACCAAC |
| | CGCTTTAACTCGTCAGGGTGTCCAGGGCGCCTGTCCGCCTGCCGCAACATCGAGGCTTTCCGGATAG |
| | GGTGGGGCACCCTACAGTACGAGGATAATGTCACCAATCCAGAGGATATGAGGCCGTACTGCTGGCA |
| | CTACCCCCCAAAGCCGTGTGGCGTAGTCCCCGCGAGGTCTGTGTGTGGCCCAGTGTACTGTTTCACC |
| | CCCAGCCCGGTAGTAGTGGGCACGACCGACAGACGTGGAGTGCCCACCTACACATGGGGAGAGAATG |
| | AGACAGATGTCTTCCTACTGAACAGCACCCGACCGCCGCAGGGCTCATGGTTCGGCTGCACGTGGAT |
| | GAACTCCACTGGTTTCACCAAGACTTGTGGCGCGCCACCTTGCCGCACCAGAGCTGACTTCAACGCC |
| | AGCACGGACTTGTTGTGCCCTACGGATTGTTTTAGGAAGCATCCTGATGCCACTTATATTAAGTGTG |
| | GTTCTGGGCCCTGGCTCACACCAAAGTGCCTGGTCCACTACCCTTACAGACTCTGGCATTACCCCTG |
| | CACAGTCAATTTTACCATCTTCAAGATAAGAATGTATGTAGGGGGGGTTGAGCACAGGCTCACGGCC |
| | GCATGCAACTTCACTCGTGGGGATCGCTGCGACTTGGAGGACAGGGACAGGAGTCAGCTGTCTCCTC |
| | TGTTGCACTCTACCACGGAATGGGCCATCCTGCCCTGCACCTACTCAGACTTACCCGCTTTGTCAAC |
| | TGGTCTTCTCCACCTTCACCAGAACATCGTGGACGTACAATACATGTATGGCCTCTCACCTGCTATC |
| | ACAAAATACGTCGTTCGATGGGAGTGGGTGGTACTCTTATTCCTGCTCTTAGCGGACGCCAGAGTCT |
| | GCGCCTGCTTGTGGATGCTCATCTTGTTGGGCCAGGCCGAAGCAGCATTGGAGAAGTTGGTCGTCTT |
| | GCACGCTGCGAGTGCGGCTAACTGCCATGGCCTCCTATATTTTGCCATCTTCTTCGTGGCAGCTTGG |
| | CACATCAGGGGTCGGGTGGTCCCCTTGACCACCTATTGCCTCACTGGCCTATGGCCCTTCTGCCTAC |
| | TGCTCATGGCACTGCCCCGGCAGGCTTATGCCTATGACGCACCTGTGCACGGACAGATAGGCGTGGG |
| | TTTGTTGATATTGATCACCCTCTTCACACTCACCCCGGGGTATAAGACCCTCCTCGGCCAGTGTCTG |
| | TGGTGGTTGTGCTATCTCCTGACCCTGGGGGAAGCCATGATTCAGGAGTGGGTACCACCCATGCAGG |
| | TGCGCGGCGGCCGCGATGGCATCGCGTGGGCCGTCACTATATTCTGCCCGGGTGTGGTGTTTGACAT |
| | TACCAAATGGCTTTTGGCGTTGCTTGGGCCTGCTTACCTCTTAAGGGCCGCTTTGACACATGTGCCG |
| | TACTTCGTCAGAGCTCACGCTCTGATAAGGGTATGCGCTTTGGTGAAGCAGCTCGCGGGGGGTAGGT |
| | ATGTTCAGGTGGCGCTATTGGCCCTTGGCAGGTGGACTGGCACCTACATCTATGACCACCTCACACC |
| | TATGTCGGACTGGGCCGCTAGCGGCCTGCGCGACTTAGCGGTCGCCGTGGAACCCATCATCTTCAGT |
| | CCGATGGAGAAGAAGGTCATCGTCTGGGGAGCGGAGACGGCTGCATGTGGGGACATTCTACATGGAC |
| | TTCCCGTGTCCGCCCGACTCGGCCAGGAGATCCTCCTCGGCCCAGCTGATGGCTACACCTCCAAGGG |
| | GTGGAAGCTCCTTGCTCCCATCACTGCTTATGCCCAGCAAACACGAGGCCTCCTGGGCGCCATAGTG |
| | GTGAGTATGACGGGGCGTGACAGGACAGAACAGGCCGGGGAAGTCCAAATCCTGTCCACAGTCTCTC |
| | AGTCCTTCCTCGGAACAACCATCTCGGGGGTTTTGTGGACTGTTTACCACGGAGCTGGCAACAAGAC |
| | TCTAGCCGGCTTACGGGGTCCGGTCACGCAGATGTACTCGAGTGCTGAGGGGGACTTGGTAGGCTGG |
| | CCCAGCCCCCCTGGGACCAAGTCTTTGGAGCCGTGCAAGTGTGGAGCCGTCGACCTATATCTGGTCA |
| | CGCGGAACGCTGATGTCATCCCGGCTCGGAGACGCGGGGACAAGCGGGGAGCATTGCTCTCCCCGAG |
| | ACCCATTTCGACCTTGAAGGGGTCCTCGGGGGGGCCGGTGCTCTGCCCTAGGGGCCACGTCGTTGGG |
| | CTCTTCCGAGCAGCTGTGTGCTCTCGGGGCGTGGCCAAATCCATCGATTTCATCCCCGTTGAGACAC |
| | TCGACGTTGTTACAAGGTCTCCCACTTTCAGTGACAACAGCACGCCACCGGCTGTGCCCCAGACCTA |
| | TCAGGTCGGGTACTTGCATGCTCCAACTGGCAGTGGAAAGAGCACCAAGGTCCCTGTCGCGTATGCC |
| | GCCCAGGGGTACAAAGTACTAGTGCTTAACCCCTCGGTAGCTGCCACCCTGGGGTTTGGGGCGTACC |
| | TATCCAAGGCACATGGCATCAATCCCAACATTAGGACTGGAGTCAGGACCGTGATGACCGGGGAGGC |
| | CATCACGTACTCCACATATGGCAAATTTCTCGCCGATGGGGGCTGCGCTAGCGGCGCCTATGACATC |
| | ATCATATGCGATGAATGCCACGCTGTGGATGCTACCTCCATTCTCGGCATCGGAACGGTCCTTGATC |
| | AAGCAGAGACAGCCGGGGTCAGACTAACTGTGCTGGCTACGGCCACACCCCCCGGGTCAGTGACAAC |
| | CCCCCATCCCGATATAGAAGAGGTAGGCCTCGGGCGGGAGGGTGAGATCCCCTTCTATGGGAGGGCG |
| | ATTCCCCTATCCTGCATCAAGGGAGGGAGACACCTGATTTTCTGCCACTCAAAGAAAAAGTGTGACG |
| | AGCTCGCGGCGGCCCTTCGGGGCATGGGCTTGAATGCCGTGGCATACTATAGAGGGTTGGACGTCTC |
| | CATAATACCAGCTCAGGGAGATGTGGTGGTCGTCGCCACCGACGCCCTCATGACGGGGTACACTGGA |
| | GACTTTGACTCCGTGATCGACTGCAATGTAGCGGTCACCCAAGCTGTCGACTTCAGCCTGGACCCCA |
| | CCTTCACTATAACCACACAGACTGTCCCACAAGACGCTGTCTCACGCAGTCAGCGCCGCGGGCGCAC |
| | AGGTAGAGGAAGACAGGGCACTTATAGGTATGTTTCCACTGGTGAACGAGCCTCAGGAATGTTTGAC |
| | AGTGTAGTGCTTTGTGAGTGCTACGACGCAGGGGCTGCGTGGTACGATCTCACACCAGCGGAGACCA |
| | CCGTCAGGCTTAGAGCGTATTTCAACACGCCCGGCCTACCCGTGTGTCAAGACCATCTTGAATTTTG |
| | GGAGGCAGTTTTCACCGGCCTCACACACATAGACGCCCACTTCCTCTCCCAAACAAAGCAAGCGGGG |
| | GAGAACTTCGCGTACCTAGTAGCCTACCAAGCTACGGTGTGCGCCAGAGCCAAGGCCCCTCCCCCGT |
| | CCTGGGACGCCATGTGGAAGTGCCTGGCCCGACTCAAGCCTACGCTTGCGGGCCCCACACCTCTCCT |
| | GTACCGTTTGGGCCCTATTACCAATGAGGTCACCCTCACACACCCTGGGACGAAGTACATCGCCACA |
| | TGCATGCAAGCTGACCTTGAGGTCATGACCAGCACGTGGGTCCTAGCTGGAGGAGTCCTGGCAGCCG |
| | TCGCCGCATATTGCCTGGCGACTGGATGCGTTTCCATCATCGGCCGCTTGCACGTCAACCAGCGAGT |
| | CGTCGTTGCGCCGGATAAGGAGGTCCTGTATGAGGCTTTTGATGAGATGGAGGAATGCGCCTCTAGG |
| | GCGGCTCTCATCGAAGAGGGGCAGCGGATAGCCGAGATGTTGAAGTCCAAGATCCAAGGCTTGCTGC |
| | AGCAGGCCTCTAAGCAGGCCCAGGACATACAACCCGCTATGCAGGCTTCATGGCCCAAAGTGGAACA |
| | ATTTTGGGCCAGACACATGTGGAACTTCATTAGCGGCATCCAATACCTCGCAGGATTGTCAACACTG |
| | CCAGGGAACCCCGCGGTGGCTTCCATGATGGCATTCAGTGCCGCCCTCACCAGTCCGTTGTCGACCA |
| | GTACCACCATCCTTCTCAACATCATGGGAGGCTGGTTAGCGTCCCAGATCGCACCACCCGCGGGGGC |
| | CACCGGCTTTGTCGTCAGTGGCCTGGTGGGGGCTGCCGTGGGCAGCATAGGCCTGGGTAAGGTGCTG |
| | GTGGACATCCTGGCAGGATATGGTGCGGGCATTTCGGGGGCCCTCGTCGCATTCAAGATCATGTCTG |
| | GCGAGAAGCCCTCTATGGAAGATGTCATCAATCTACTGCCTGGGATCCTGTCTCCGGGAGCCCTGGT |
| | GGTGGGGGTCATCTGCGCGGCCATTCTGCGCCGCCACGTGGGACCGGGGGAGGGCGCGGTCCAATGG |
| | ATGAACAGGCTTATTGCCTTTGCTTCCAGAGGAAACCACGTCGCCCCTACTCACTACGTGACGGAGT |
| | CGGATGCGTCGCAGCGTGTGACCCAACTACTTGGCTCTCTTACTATAACCAGCCTACTCAGAAGACT |
| | CCACAATTGGATAACTGAGGACTGCCCCATCCCATGCTCCGGATCCTGGCTCCGCGACGTGTGGGAC |
| | TGGGTTTGCACCATCTTGACAGACTTCAAAAATTGGCTGACCTCTAAATTGTTCCCCAAGCTGCCCG |
| | GCCTCCCCTTCATCTCTTGTCAAAAGGGGTACAAGGGTGTGTGGGCCGGCACTGGCATCATGACCAC |
| | GCGCTGCCCTTGCGGCGCCAACATCTCTGGCAATGTCCGCCTGGGCTCTATGAGGATCACAGGGCCT |
| | AAAACCTGCATGAACACCTGGCAGGGGACCTTTCCTATCAATTGCTACACGGAGGGCCAGTGCGCGC |
| | CGAAACCCCCCACGAACTACAAGACCGCCATCTGGAGGGTGGCGGCCTCGGAGTACGCGGAGGTGAC |
| | GCAGCATGGGTCGTACTCCTATGTAACAGGACTGACCACTGACAATCTGAAAATTCCTTGCCAACTA |
| | CCTTCTCCAGAGTTTTTCTCCTGGGTGGACGGTGTGCAGATCCATAGGTTTGCACCCACACCAAAGC |
| | CGTTTTTCCGGGATGAGGTCTCGTTCTGCGTTGGGCTTAATTCCTATGCTGTCGGGTCCCAGCTTCC |
| | CTGTGAACCTGAGCCCGACGCAGACGTATTGAGGTCCATGCTAACAGATCCGCCCCACATCACGGCG |
| | GAGACTGCGGCGCGGCGCTTGGCACGGGGATCACCTCCATCTGAGGCGAGCTCCTCAGTGAGCCAGC |
| | TATCAGCACCGTCGCTGCGGGCCACCTGCACCACCCACAGCAACACCTATGACGTGGACATGGTCGA |
| | TGCCAACCTGCTCATGGAGGGCGGTGTGGCTCAGACAGAGCCTGAGTCCAGGGTGCCCGTTCTGGAC |
| | TTTCTCGAGCCAATGGCCGAGGAAGAGAGCGACCTTGAGCCCTCAATACCATCGGAGTGCATGCTCC |
| | CCAGGAGCGGGTTTCCACGGGCCTTACCGGCTTGGGCACGGCCTGACTACAACCCGCCGCTCGTGGA |
| | ATCGTGGAGGAGGCCAGATTACCAACCGCCCACCGTTGCTGGTTGTGCTCTCCCCCCCCCCAAGAAG |
| | GCCCCGACGCCTCCCCCAAGGAGACGCCGGACAGTGGGTCTGAGCGAGAGCACCATATCAGAAGCCC |
| | TCCAGCAACTGGCCATCAAGACCTTTGGCCAGCCCCCCTCGAGCGGTGATGCAGGCTCGTCCACGGG |
| | GGCGGGCGCCGCCGAATCCGGCGGTCCGACGTCCCCTGGTGAGCCGGCCCCCTCAGAGACAGGTTCC |
| | GCCTCCTCTATGCCCCCCCTCGAGGGGGAGCCTGGAGATCCGGACCTGGAGTCTGATCAGGTAGAGC |
| | TTCAACCTCCCCCCCAGGGGGGGGGGGTAGCTCCCGGTTCGGGCTCGGGGTCTTGGTCTACTTGCTC |
| | CGAGGAGGACGATACCACCGTGTGCTGCTCCATGTCATACTCCTGGACCGGGGCTCTAATAACTCCC |
| | TGTAGCCCCGAAGAGGAAAAGTTGCCAATCAACCCTTTGAGTAACTCGCTGTTGCGATACCATAACA |
| | AGGTGTACTGTACAACATCAAAGAGCGCCTCACAGAGGGCTAAAAAGGTAACTTTTGACAGGACGCA |
| | AGTGCTCGACGCCCATTATGACTCAGTCTTAAAGGACATCAAGCTAGCGGCTTCCAAGGTCAGCGCA |
| | AGGCTCCTCACCTTGGAGGAGGCGTGCCAGTTGACTCCACCCCATTCTGCAAGATCCAAGTATGGAT |
| | TCGGGGCCAAGGAGGTCCGCAGCTTGTCCGGGAGGGCCGTTAACCACATCAAGTCCGTGTGGAAGGA |
| | CCTCCTGGAAGACCCACAAACACCAATTCCCACAACCATCATGGCCAAAAATGAGGTGTTCTGCGTG |
| | GACCCCGCCAAGGGGGGTAAGAAACCAGCTCGCCTCATCGTTTACCCTGACCTCGGCGTCCGGGTCT |
| | GCGAGAAAATGGCCCTCTATGACATTACACAAAAGCTTCCTCAGGCGGTAATGGGAGCTTCCTATGG |
| | CTTCCAGTACTCCCCTGCCCAACGGGTGGAGTATCTCTTGAAAGCATGGGCGGAAAAGAAGGACCCC |
| | ATGGGTTTTTCGTATGATACCCGATGCTTCGACTCAACCGTCACTGAGAGAGACATCAGGACCGAGG |
| | AGTCCATATACCAGGCCTGCTCCCTGCCCGAGGAGGCCCGCACTGCCATACACTCGCTGACTGAGAG |
| | ACTTTACGTAGGAGGGCCCATGTTCAACAGCAAGGGTCAAACCTGCGGTTACAGACGTTGCCGCGCC |
| | AGCGGGGTGCTAACCACTAGCATGGGTAACACCATCACATGCTATGTGAAAGCCCTAGCGGCCTGCA |
| | AGGCTGCGGGGATAGTTGCGCCCACAATGCTGGTATGCGGCGATGACCTAGTAGTCATCTCAGAAAG |
| | CCAGGGGACTGAGGAGGACGAGCGGAACCTGAGAGCCTTCACGGAGGCCATGACCAGGTACTCTGCC |
| | CCTCCTGGTGATCCCCCCAGACCGGAATATGACCTGGAGCTAATAACATCCTGTTCCTCAAATGTGT |
| | CTGTGGCGTTGGGCCCGCGGGGCCGCCGCAGATACTACCTGACCAGAGACCCAACCACTCCACTCGC |
| | CCGGGCTGCCTGGGAAACAGTTAGACACTCCCCTATCAATTCATGGCTGGGAAACATCATCCAGTAT |
| | GCTCCAACCATATGGGTTCGCATGGTCCTAATGACACACTTCTTCTCCATTCTCATGGTCCAAGACA |
| | CCCTGGACCAGAACCTCAACTTTGAGATGTATGGATCAGTATACTCCGTGAATCCTTTGGACCTTCC |
| | AGCCATAATTGAGAGGTTACACGGGCTTGACGCCTTTTCTATGCACACATACTCTCACCACGAACTG |
| | ACGCGGGTGGCTTCAGCCCTCAGAAAACTTGGGGCGCCACCCCTCAGGGTGTGGAAGAGTCGGGCTC |
| | GCGCAGTCAGGGCGTCCCTCATCTCCCGTGGAGGGAAAGCGGCCGTTTGCGGCCGATATCTCTTCAA |
| | TTGGGCGGTGAAGACCAAGCTCAAACTCACTCCATTGCCGGAGGCGCGCCTACTGGACTTATCCAGT |
| | TGGTTCACCGTCGGCGCCGGCGGGGGCGACATTTTTCACAGCGTGTCGCGCGCCCGACCCCGCTCAT |
| | TACTCTTCGGCCTACTCCTACTTTTCGTAGGGGTAGGCCTCTTCCTACTCCCCGCTCGGTAGAGCGG |
| | CACACACTAGGTACACTCCATAGCTAACTGTTCCTTTTTTTTTTTTTTTTTTTTTTTTTTTTTTTTT |
| | TTTTTTTTTTCTTTTTTTTTTTTTTCCCTCTTTCTTCCCTTCTCATCTTATTCTACTTTCTTTCTTG |
| | GTGGCTCCATCTTAGCCCTAGTCACGGCTAGCTGTGAAAGGTCCGTGAGCCGCATGACTGCAGAGAG |
| | TGCCGTAACTGGTCTCTCTGCAGATCATGT |
| 301 | GenBank Accession No. NC_004102.1 (Hepatitis C virus genotype 1, complete |
| | genome) |
| | GCCAGCCCCCTGATGGGGGCGACACTCCACCATGAATCACTCCCCTGTGAGGAACTACTGTCTTCAC |
| | GCAGAAAGCGTCTAGCCATGGCGTTAGTATGAGTGTCGTGCAGCCTCCAGGACCCCCCCTCCCGGGA |
| | GAGCCATAGTGGTCTGCGGAACCGGTGAGTACACCGGAATTGCCAGGACGACCGGGTCCTTTCTTGG |
| | ATAAACCCGCTCAATGCCTGGAGATTTGGGCGTGCCCCCGCAAGACTGCTAGCCGAGTAGTGTTGGG |
| | TCGCGAAAGGCCTTGTGGTACTGCCTGATAGGGTGCTTGCGAGTGCCCCGGGAGGTCTCGTAGACCG |
| | TGCACCATGAGCACGAATCCTAAACCTCAAAGAAAAACCAAACGTAACACCAACCGTCGCCCACAGG |
| | ACGTCAAGTTCCCGGGTGGCGGTCAGATCGTTGGTGGAGTTTACTTGTTGCCGCGCAGGGGCCCTAG |
| | ATTGGGTGTGCGCGCGACGAGGAAGACTTCCGAGCGGTCGCAACCTCGAGGTAGACGTCAGCCTATC |
| | CCCAAGGCACGTCGGCCCGAGGGCAGGACCTGGGCTCAGCCCGGGTACCCTTGGCCCCTCTATGGCA |
| | ATGAGGGTTGCGGGTGGGCGGGATGGCTCCTGTCTCCCCGTGGCTCTCGGCCTAGCTGGGGCCCCAC |
| | AGACCCCCGGCGTAGGTCGCGCAATTTGGGTAAGGTCATCGATACCCTTACGTGCGGCTTCGCCGAC |
| | CTCATGGGGTACATACCGCTCGTCGGCGCCCCTCTTGGAGGCGCTGCCAGGGCCCTGGCGCATGGCG |
| | TCCGGGTTCTGGAAGACGGCGTGAACTATGCAACAGGGAACCTTCCTGGTTGCTCTTTCTCTATCTT |
| | CCTTCTGGCCCTGCTCTCTTGCCTGACTGTGCCCGCTTCAGCCTACCAAGTGCGCAATTCCTCGGGG |
| | CTTTACCATGTCACCAATGATTGCCCTAACTCGAGTATTGTGTACGAGGCGGCCGATGCCATCCTGC |
| | ACACTCCGGGGTGTGTCCCTTGCGTTCGCGAGGGTAACGCCTCGAGGTGTTGGGTGGCGGTGACCCC |
| | CACGGTGGCCACCAGGGACGGCAAACTCCCCACAACGCAGCTTCGACGTCATATCGATCTGCTTGTC |
| | GGGAGCGCCACCCTCTGCTCGGCCCTCTACGTGGGGGACCTGTGCGGGTCTGTCTTTCTTGTTGGTC |
| | AACTGTTTACCTTCTCTCCCAGGCGCCACTGGACGACGCAAGACTGCAATTGTTCTATCTATCCCGG |
| | CCATATAACGGGTCATCGCATGGCATGGGATATGATGATGAACTGGTCCCCTACGGCAGCGTTGGTG |
| | GTAGCTCAGCTGCTCCGGATCCCACAAGCCATCATGGACATGATCGCTGGTGCTCACTGGGGAGTCC |
| | TGGCGGGCATAGCGTATTTCTCCATGGTGGGGAACTGGGCGAAGGTCCTGGTAGTGCTGCTGCTATT |
| | TGCCGGCGTCGACGCGGAAACCCACGTCACCGGGGGAAGTGCCGGCCGCACCACGGCTGGGCTTGTT |
| | GGTCTCCTTACACCAGGCGCCAAGCAGAACATCCAACTGATCAACACCAACGGCAGTTGGCACATCA |
| | ATAGCACGGCCTTGAACTGCAATGAAAGCCTTAACACCGGCTGGTTAGCAGGGCTCTTCTATCAGCA |
| | CAAATTCAACTCTTCAGGCTGTCCTGAGAGGTTGGCCAGCTGCCGACGCCTTACCGATTTTGCCCAG |
| | GGCTGGGGTCCTATCAGTTATGCCAACGGAAGCGGCCTCGACGAACGCCCCTACTGCTGGCACTACC |
| | CTCCAAGACCTTGTGGCATTGTGCCCGCAAAGAGCGTGTGTGGCCCGGTATATTGCTTCACTCCCAG |
| | CCCCGTGGTGGTGGGAACGACCGACAGGTCGGGCGCGCCTACCTACAGCTGGGGTGCAAATGATACG |
| | GATGTCTTCGTCCTTAACAACACCAGGCCACCGCTGGGCAATTGGTTCGGTTGTACCTGGATGAACT |
| | CAACTGGATTCACCAAAGTGTGCGGAGCGCCCCCTTGTGTCATCGGAGGGGTGGGCAACAACACCTT |
| | GCTCTGCCCCACTGATTGTTTCCGCAAGCATCCGGAAGCCACATACTCTCGGTGCGGCTCCGGTCCC |
| | TGGATTACACCCAGGTGCATGGTCGACTACCCGTATAGGCTTTGGCACTATCCTTGTACCATCAATT |
| | ACACCATATTCAAAGTCAGGATGTACGTGGGAGGGGTCGAGCACAGGCTGGAAGCGGCCTGCAACTG |
| | GACGCGGGGCGAACGCTGTGATCTGGAAGACAGGGACAGGTCCGAGCTCAGCCCATTGCTGCTGTCC |
| | ACCACACAGTGGCAGGTCCTTCCGTGTTCTTTCACGACCCTGCCAGCCTTGTCCACCGGCCTCATCC |
| | ACCTCCACCAGAACATTGTGGACGTGCAGTACTTGTACGGGGTAGGGTCAAGCATCGCGTCCTGGGC |
| | CATTAAGTGGGAGTACGTCGTTCTCCTGTTCCTCCTGCTTGCAGACGCGCGCGTCTGCTCCTGCTTG |
| | TGGATGATGTTACTCATATCCCAAGCGGAGGCGGCTTTGGAGAACCTCGTAATACTCAATGCAGCAT |
| | CCCTGGCCGGGACGCACGGTCTTGTGTCCTTCCTCGTGTTCTTCTGCTTTGCGTGGTATCTGAAGGG |
| | TAGGTGGGTGCCCGGAGCGGTCTACGCCTTCTACGGGATGTGGCCTCTCCTCCTGCTCCTGCTGGCG |
| | TTGCCTCAGCGGGCATACGCACTGGACACGGAGGTGGCCGCGTCGTGTGGCGGCGTTGTTCTTGTCG |
| | GGTTAATGGCGCTGACTCTGTCGCCATATTACAAGCGCTACATCAGCTGGTGCATGTGGTGGCTTCA |
| | GTATTTTCTGACCAGAGTAGAAGCGCAACTGCACGTGTGGGTTCCCCCCCTCAACGTCCGGGGGGGG |
| | CGCGATGCCGTCATCTTACTCATGTGTGTTGTACACCCGACTCTGGTATTTGACATCACCAAACTAC |
| | TCCTGGCCATCTTCGGACCCCTTTGGATTCTTCAAGCCAGTTTGCTTAAAGTCCCCTACTTCGTGCG |
| | CGTTCAAGGCCTTCTCCGGATCTGCGCGCTAGCGCGGAAGATAGCCGGAGGTCATTACGTGCAAATG |
| | GCCATCATCAAGTTAGGGGCGCTTACTGGCACCTATGTGTATAACCATCTCACCCCTCTTCGAGACT |
| | GGGCGCACAACGGCCTGCGAGATCTGGCCGTGGCTGTGGAACCAGTCGTCTTCTCCCGAATGGAGAC |
| | CAAGCTCATCACGTGGGGGGCAGATACCGCCGCGTGCGGTGACATCATCAACGGCTTGCCCGTCTCT |
| | GCCCGTAGGGGCCAGGAGATACTGCTTGGGCCAGCCGACGGAATGGTCTCCAAGGGGTGGAGGTTGC |
| | TGGCGCCCATCACGGCGTACGCCCAGCAGACGAGAGGCCTCCTAGGGTGTATAATCACCAGCCTGAC |
| | TGGCCGGGACAAAAACCAAGTGGAGGGTGAGGTCCAGATCGTGTCAACTGCTACCCAAACCTTCCTG |
| | GCAACGTGCATCAATGGGGTATGCTGGACTGTCTACCACGGGGCCGGAACGAGGACCATCGCATCAC |
| | CCAAGGGTCCTGTCATCCAGATGTATACCAATGTGGACCAAGACCTTGTGGGCTGGCCCGCTCCTCA |
| | AGGTTCCCGCTCATTGACACCCTGCACCTGCGGCTCCTCGGACCTTTACCTGGTCACGAGGCACGCC |
| | GATGTCATTCCCGTGCGCCGGCGAGGTGATAGCAGGGGTAGCCTGCTTTCGCCCCGGCCCATTTCCT |
| | ACTTGAAAGGCTCCTCGGGGGGTCCGCTGTTGTGCCCCGCGGGACACGCCGTGGGCCTATTCAGGGC |
| | CGCGGTGTGCACCCGTGGAGTGGCTAAGGCGGTGGACTTTATCCCTGTGGAGAACCTAGAGACAACC |
| | ATGAGATCCCCGGTGTTCACGGACAACTCCTCTCCACCAGCAGTGCCCCAGAGCTTCCAGGTGGCCC |
| | ACCTGCATGCTCCCACCGGCAGCGGTAAGAGCACCAAGGTCCCGGCTGCGTACGCAGCCCAGGGCTA |
| | CAAGGTGTTGGTGCTCAACCCCTCTGTTGCTGCAACGCTGGGCTTTGGTGCTTACATGTCCAAGGCC |
| | CATGGGGTTGATCCTAATATCAGGACCGGGGTGAGAACAATTACCACTGGCAGCCCCATCACGTACT |
| | CCACCTACGGCAAGTTCCTTGCCGACGGCGGGTGCTCAGGAGGTGCTTATGACATAATAATTTGTGA |
| | CGAGTGCCACTCCACGGATGCCACATCCATCTTGGGCATCGGCACTGTCCTTGACCAAGCAGAGACT |
| | GCGGGGGCGAGACTGGTTGTGCTCGCCACTGCTACCCCTCCGGGCTCCGTCACTGTGTCCCATCCTA |
| | ACATCGAGGAGGTTGCTCTGTCCACCACCGGAGAGATCCCTTTTTACGGCAAGGCTATCCCCCTCGA |
| | GGTGATCAAGGGGGGAAGACATCTCATCTTCTGCCACTCAAAGAAGAAGTGCGACGAGCTCGCCGCG |
| | AAGCTGGTCGCATTGGGCATCAATGCCGTGGCCTACTACCGCGGTCTTGACGTGTCTGTCATCCCGA |
| | CCAGCGGCGATGTTGTCGTCGTGTCGACCGATGCTCTCATGACTGGCTTTACCGGCGACTTCGACTC |
| | TGTGATAGACTGCAACACGTGTGTCACTCAGACAGTCGATTTCAGCCTTGACCCTACCTTTACCATT |
| | GAGACAACCACGCTCCCCCAGGATGCTGTCTCCAGGACTCAACGCCGGGGCAGGACTGGCAGGGGGA |
| | AGCCAGGCATCTACAGATTTGTGGCACCGGGGGAGCGCCCCTCCGGCATGTTCGACTCGTCCGTCCT |
| | CTGTGAGTGCTATGACGCGGGCTGTGCTTGGTATGAGCTCACGCCCGCCGAGACTACAGTTAGGCTA |
| | CGAGCGTACATGAACACCCCGGGGCTTCCCGTGTGCCAGGACCATCTTGAATTTTGGGAGGGCGTCT |
| | TTACGGGCCTCACTCATATAGATGCCCACTTTCTATCCCAGACAAAGCAGAGTGGGGAGAACTTTCC |
| | TTACCTGGTAGCGTACCAAGCCACCGTGTGCGCTAGGGCTCAAGCCCCTCCCCCATCGTGGGACCAG |
| | ATGTGGAAGTGTTTGATCCGCCTTAAACCCACCCTCCATGGGCCAACACCCCTGCTATACAGACTGG |
| | GCGCTGTTCAGAATGAAGTCACCCTGACGCACCCAATCACCAAATACATCATGACATGCATGTCGGC |
| | CGACCTGGAGGTCGTCACGAGCACCTGGGTGCTCGTTGGCGGCGTCCTGGCTGCTCTGGCCGCGTAT |
| | TGCCTGTCAACAGGCTGCGTGGTCATAGTGGGCAGGATTGTCTTGTCCGGGAAGCCGGCAATTATAC |
| | CTGACAGGGAGGTTCTCTACCAGGAGTTCGATGAGATGGAAGAGTGCTCTCAGCACTTACCGTACAT |
| | CGAGCAAGGGATGATGCTCGCTGAGCAGTTCAAGCAGAAGGCCCTCGGCCTCCTGCAGACCGCGTCC |
| | CGCCAAGCAGAGGTTATCACCCCTGCTGTCCAGACCAACTGGCAGAAACTCGAGGTCTTCTGGGCGA |
| | AGCACATGTGGAATTTCATCAGTGGGATACAATACTTGGCGGGCCTGTCAACGCTGCCTGGTAACCC |
| | CGCCATTGCTTCATTGATGGCTTTTACAGCTGCCGTCACCAGCCCACTAACCACTGGCCAAACCCTC |
| | CTCTTCAACATATTGGGGGGGTGGGTGGCTGCCCAGCTCGCCGCCCCCGGTGCCGCTACCGCCTTTG |
| | TGGGCGCTGGCTTAGCTGGCGCCGCCATCGGCAGCGTTGGACTGGGGAAGGTCCTCGTGGACATTCT |
| | TGCAGGGTATGGCGCGGGCGTGGCGGGAGCTCTTGTAGCATTCAAGATCATGAGCGGTGAGGTCCCC |
| | TCCACGGAGGACCTGGTCAATCTGCTGCCCGCCATCCTCTCGCCTGGAGCCCTTGTAGTCGGTGTGG |
| | TCTGCGCAGCAATACTGCGCCGGCACGTTGGCCCGGGCGAGGGGGCAGTGCAATGGATGAACCGGCT |
| | AATAGCCTTCGCCTCCCGGGGGAACCATGTTTCCCCCACGCACTACGTGCCGGAGAGCGATGCAGCC |
| | GCCCGCGTCACTGCCATACTCAGCAGCCTCACTGTAACCCAGCTCCTGAGGCGACTGCATCAGTGGA |
| | TAAGCTCGGAGTGTACCACTCCATGCTCCGGTTCCTGGCTAAGGGACATCTGGGACTGGATATGCGA |
| | GGTGCTGAGCGACTTTAAGACCTGGCTGAAAGCCAAGCTCATGCCACAACTGCCTGGGATTCCCTTT |
| | GTGTCCTGCCAGCGCGGGTATAGGGGGGTCTGGCGAGGAGACGGCATTATGCACACTCGCTGCCACT |
| | GTGGAGCTGAGATCACTGGACATGTCAAAAACGGGACGATGAGGATCGTCGGTCCTAGGACCTGCAG |
| | GAACATGTGGAGTGGGACGTTCCCCATTAACGCCTACACCACGGGCCCCTGTACTCCCCTTCCTGCG |
| | CCGAACTATAAGTTCGCGCTGTGGAGGGTGTCTGCAGAGGAATACGTGGAGATAAGGCGGGTGGGGG |
| | ACTTCCACTACGTATCGGGTATGACTACTGACAATCTTAAATGCCCGTGCCAGATCCCATCGCCCGA |
| | ATTTTTCACAGAATTGGACGGGGTGCGCCTACATAGGTTTGCGCCCCCTTGCAAGCCCTTGCTGCGG |
| | GAGGAGGTATCATTCAGAGTAGGACTCCACGAGTACCCGGTGGGGTCGCAATTACCTTGCGAGCCCG |
| | AACCGGACGTAGCCGTGTTGACGTCCATGCTCACTGATCCCTCCCATATAACAGCAGAGGCGGCCGG |
| | GAGAAGGTTGGCGAGAGGGTCACCCCCTTCTATGGCCAGCTCCTCGGCCAGCCAGCTGTCCGCTCCA |
| | TCTCTCAAGGCAACTTGCACCGCCAACCATGACTCCCCTGACGCCGAGCTCATAGAGGCTAACCTCC |
| | TGTGGAGGCAGGAGATGGGCGGCAACATCACCAGGGTTGAGTCAGAGAACAAAGTGGTGATTCTGGA |
| | CTCCTTCGATCCGCTTGTGGCAGAGGAGGATGAGCGGGAGGTCTCCGTACCCGCAGAAATTCTGCGG |
| | AAGTCTCGGAGATTCGCCCGGGCCCTGCCCGTTTGGGCGCGGCCGGACTACAACCCCCCGCTAGTAG |
| | AGACGTGGAAAAAGCCTGACTACGAACCACCTGTGGTCCATGGCTGCCCGCTACCACCTCCACGGTC |
| | CCCTCCTGTGCCTCCGCCTCGGAAAAAGCGTACGGTGGTCCTCACCGAATCAACCCTATCTACTGCC |
| | TTGGCCGAGCTTGCCACCAAAAGTTTTGGCAGCTCCTCAACTTCCGGCATTACGGGCGACAATACGA |
| | CAACATCCTCTGAGCCCGCCCCTTCTGGCTGCCCCCCCGACTCCGACGTTGAGTCCTATTCTTCCAT |
| | GCCCCCCCTGGAGGGGGAGCCTGGGGATCCGGATCTCAGCGACGGGTCATGGTCGACGGTCAGTAGT |
| | GGGGCCGACACGGAAGATGTCGTGTGCTGCTCAATGTCTTATTCCTGGACAGGCGCACTCGTCACCC |
| | CGTGCGCTGCGGAAGAACAAAAACTGCCCATCAACGCACTGAGCAACTCGTTGCTACGCCATCACAA |
| | TCTGGTGTATTCCACCACTTCACGCAGTGCTTGCCAAAGGCAGAAGAAAGTCACATTTGACAGACTG |
| | CAAGTTCTGGACAGCCATTACCAGGACGTGCTCAAGGAGGTCAAAGCAGCGGCGTCAAAAGTGAAGG |
| | CTAACTTGCTATCCGTAGAGGAAGCTTGCAGCCTGACGCCCCCACATTCAGCCAAATCCAAGTTTGG |
| | CTATGGGGCAAAAGACGTCCGTTGCCATGCCAGAAAGGCCGTAGCCCACATCAACTCCGTGTGGAAA |
| | GACCTTCTGGAAGACAGTGTAACACCAATAGACACTACCATCATGGCCAAGAACGAGGTTTTCTGCG |
| | TTCAGCCTGAGAAGGGGGGTCGTAAGCCAGCTCGTCTCATCGTGTTCCCCGACCTGGGCGTGCGCGT |
| | GTGCGAGAAGATGGCCCTGTACGACGTGGTTAGCAAGCTCCCCCTGGCCGTGATGGGAAGCTCCTAC |
| | GGATTCCAATACTCACCAGGACAGCGGGTTGAATTCCTCGTGCAAGCGTGGAAGTCCAAGAAGACCC |
| | CGATGGGGTTCTCGTATGATACCCGCTGTTTTGACTCCACAGTCACTGAGAGCGACATCCGTACGGA |
| | GGAGGCAATTTACCAATGTTGTGACCTGGACCCCCAAGCCCGCGTGGCCATCAAGTCCCTCACTGAG |
| | AGGCTTTATGTTGGGGGCCCTCTTACCAATTCAAGGGGGGAAAACTGCGGCTACCGCAGGTGCCGCG |
| | CGAGCGGCGTACTGACAACTAGCTGTGGTAACACCCTCACTTGCTACATCAAGGCCCGGGCAGCCTG |
| | TCGAGCCGCAGGGCTCCAGGACTGCACCATGCTCGTGTGTGGCGACGACTTAGTCGTTATCTGTGAA |
| | AGTGCGGGGGTCCAGGAGGACGCGGCGAGCCTGAGAGCCTTCACGGAGGCTATGACCAGGTACTCCG |
| | CCCCCCCCGGGGACCCCCCACAACCAGAATACGACTTGGAGCTTATAACATCATGCTCCTCCAACGT |
| | GTCAGTCGCCCACGACGGCGCTGGAAAGAGGGTCTACTACCTTACCCGTGACCCTACAACCCCCCTC |
| | GCGAGAGCCGCGTGGGAGACAGCAAGACACACTCCAGTCAATTCCTGGCTAGGCAACATAATCATGT |
| | TTGCCCCCACACTGTGGGCGAGGATGATACTGATGACCCATTTCTTTAGCGTCCTCATAGCCAGGGA |
| | TCAGCTTGAACAGGCTCTTAACTGTGAGATCTACGGAGCCTGCTACTCCATAGAACCACTGGATCTA |
| | CCTCCAATCATTCAAAGACTCCATGGCCTCAGCGCATTTTCACTCCACAGTTACTCTCCAGGTGAAA |
| | TCAATAGGGTGGCCGCATGCCTCAGAAAACTTGGGGTCCCGCCCTTGCGAGCTTGGAGACACCGGGC |
| | CCGGAGCGTCCGCGCTAGGCTTCTGTCCAGAGGAGGCAGGGCTGCCATATGTGGCAAGTACCTCTTC |
| | AACTGGGCAGTAAGAACAAAGCTCAAACTCACTCCAATAGCGGCCGCTGGCCGGCTGGACTTGTCCG |
| | GTTGGTTCACGGCTGGCTACAGCGGGGGAGACATTTATCACAGCGTGTCTCATGCCCGGCCCCGCTG |
| | GTTCTGGTTTTGCCTACTCCTGCTCGCTGCAGGGGTAGGCATCTACCTCCTCCCCAACCGATGAAGG |
| | TTGGGGTAAACACTCCGGCCTCTTAGGCCATTTCCTGTTTTTTTTTTTTTTTTTTTTTTTTTTTTTT |
| | TTTTTTTTTTTTTTTTTTTTTCTTTTTTTTTTTTTTTTTCCTTTTTTTTTTTTTTTTTTTTCTTTCC |
| | TTCTTTTTTCCTTTCTTTTCCTTCCTTCTTTAATGGTGGCTCCATCTTAGCCCTAGTCACGGCTAGC |
| | TGTGAAAGGTCCGTGAGCCGCATGACTGCAGAGAGTGCTGATACTGGCCTCTCTGCAGATCATGT |
| 302 | GenBank Accession No. NC_030791.1 (Hepatitis C virus genotype 7, complete |
| | genome) |
| | GCCAGCCCCTTACGGGGCGACACTCCGCCATGAATCACTCCCCTGCGAGGAACCACTGTCCTCACGC |
| | AGAAAGCGTCTAGCCATGACGTTAGTATGAGTGTCGTACAGCCTCCAGGACCCCCCCTCCCGGGAGA |
| | GCCATAGTGGTCTGCGGAACCGGTGAGTACACCGGAATTGCCGGGAAGACTGGGTCCTTTCTTGGAT |
| | CAACCCACTCTATGCCCGGAGATTTGGGCGTGCCCCCGCGAGACTGCTAGCCGAGTAGTGTTGGGTC |
| | GCGAAAGGCCTTGTGGTACTGCCTGATAGGGTGCTTGCGAGTGCCCCGGGAGGTCTCGTAGACCGTG |
| | CACCATGAGCACGAATCCTAAACCTCAAAGATTAACCAAAAGAAACACCGTCCGTCGCCCACAGAAC |
| | GTTAAGTTCCCGGGTGGCGGGCAGATCGTTGGCGGAGTCTACTTGTTGCCGCGCAGGGGCCCTAGAT |
| | TGGGTGTGCGCGGCACTAGGAAGAGTTCGGAGCGATCGCAGCCCAGGGGAAGACGCCAACGTATCCC |
| | CAAAGCTGCCTCTTCACAGGGTAAAGCCTGGGGCAAGCCCGGGTACCCTTGGCCCCTGTATGGTAAC |
| | GAGGGCTGTGGCTGGGCAGGGTGGCTCCTGTCCCCCCGCGGCTCTCGACCTACTTGGGGCCCCACYG |
| | ACCCCCGGCACCGCTCGCGAAACCTCGGTAAGGTGATCGACACCATGACCTGCGGGTTTGCCGACCT |
| | CATGGGGTACATCCCTGTCCTAGGCGCCCCCCTAGGGGGCGTTGCCAGGGCTCTGGCACATGGTGTT |
| | AGAGTTCTGGAGGACGGGGTCAACTATGCAACAGGGAACTTGCCTGGTTGCTCCTTTTCTATCTTCY |
| | TACTAGCCCTCCTGTCATGTCTAACAGTCCCGGCATCGGCTTATGAAGTCCGCAACTCCAGTGGRGT |
| | CTATCATCTCACCAATGACTGCCCCAACGCTAGTATAGTCTATGAAACAGACAACGCCATCCTACAC |
| | GAGCCTGGGTGCGTGCCTTGCGTTCGCGAGGGTAATACTAGCAGGTGTTGGGAACCAGTGGCCCCCA |
| | CTTTGGCGGTCCGCTATCGCGGAGCGCTTACTGACGATTTGCGGACGCATATTGACCTAGTGGTGGC |
| | GTCAGCTACCCTGTGCTCCGCCTTGTACGTGGGGGACATTTGTGGAGCCATCTTCATTGCCAGCCAA |
| | GCTGTTCTCTGGAAGCCCGGGGGGGGTCGGATAGTGCAAGATTGCAATTGTTCGATCTACCCGGGCC |
| | ACGTCACCGGCCACAGGATGGCGTGGGACATGATGCAGAACTGGGCGCCGGCCTTGTCAATGGTTGC |
| | CGCTTACGCTGTGAGAGTGCCCGGTGTCATCATTACCACTGTAGCGGGCGGCCACTGGGGTGTGTTA |
| | TTTGGCCTCGCTTACTTTGGTATGGCGGGAAACTGGGCAAAGGTAATACTCATCATGCTACTCATGT |
| | CCGGCGTCGACGCGGAAACCATGGCCGTCGGGGCTAGGGCCGCTCACACCACTGGCGCCCTTGTCAG |
| | CCTGCTCAATCCAGGGCCCAGTCAGCGCCTGCAGCTGATCAACACCAATGGGTCATGGCACATCAAC |
| | CGGACCGCTTTGAACTGCAATGACTCTTTGCAGACAGGGTTCATAGCGGCCCTCTTCTACACACATA |
| | GGTTCAATTCTAGTGGCTGTCCCGAGAGGATGGCTTCTTGTAAACCTCTCAGTGACTTTGACCAGGG |
| | GTGGGGCCCGCTGTGGTACAATTCAACAGAAAGACCTTCGGACCAGCGACCCTATTGCTGGCACTAC |
| | GCGCCATCGCCGTGTGGTATTGTGCCGGCTAAGGATGTTTGCGGTCCGGTCTACTGCTTTACACCAA |
| | GCCCGGTTGTGGTGGGCACCACGGATCGCCGGGGGGTGCCCACGTATACTTGGGGTGAAAATGAGTC |
| | TGATGTCTTCYTGCTCAACAGCACAAGGCCGCCGCAAGGCAGTTGGTTTGGGTGCTCATGGATGAAC |
| | ACAACGGGGTTCACGAAGACCTGCGGAGGTCCTCCGTGCAAGATACGTCCCCAGGGTGCCCAGAGTA |
| | ACACCTCTCTCACTTGTCCCACAGACTGCTTCAGGAAACATCCGCGTGCCACATACTCCGCTTGCGG |
| | ATCTGGTCCGTGGTTGACACCTAGATGCATGGTCCATTACCCCTATAGACTGTGGCACTACCCGTGT |
| | ACAGTCAACTTCACCATACACAAAGTCAGGTTATACATAGGGGGTGTAGAACATAGGCTCGATGCAG |
| | CGTGCAATTGGACGCGGGGTGAGCGATGCGACCTGGAGGACCGAGACAGGGTGGACATGTCCCCCCT |
| | GCTCCATTCCACTACGGAGCTCGCAATACTTCCGTGTTCCTTTGTGCCGCTTCCGGCCTTATCTACG |
| | GGACTGATCCACCTGCACCAAAACATCGTTGACGCCCAGTACCTTTATGGTCTTTCTCCCGCTATAA |
| | TAAGCTGGGCCATCAGATGGGAGTGGGTAGTCCTCGTTTTCCTACTCCTGGCGGACGCGCGGATCTG |
| | CGCCTGCCTTTGGATGATGATGCTTATGGCCCAGGCTGAAGCCGCTCTGGAGAACTTGATCCACCTC |
| | AACGCGGCCAGCCTTGCGGGAACCCATGGTATCTGGTGGCTCCTTTTAGTCTTTTGTGCCTCTTGGC |
| | ATCTACGAGGCAGGGTTGTCCCTCTGGTGACGTATGGGATATGCGGGATGTGGCCCTTCTTCCTCAT |
| | GTTGCTGAGCCTCCCCCCACGAGCGTATGCTCTGGACAGGGAAGTGAGCGCAGCGTTGGGAACGGGC |
| | ATGCTCGCCATCATCCTATTAGTTACCTTGGGACCGCACTACAAGAGACTTCTAGCCCTTATTCTCT |
| | GGTGGGTCACATATTTCCTTACAAGGTGTGAAGCAGCACTCCAAACGTGGGTCCCTCCTCTCAACCC |
| | TCGGGGGGGCAGGGACGGTTTCATCCTGTGTGTGCTGCTGTGCTATCCAGGCCTTGTCTTTGACATC |
| | ACAAAATGGTTGCTGGTCATGATGTGCCCTCTCTACCTCCTCCAGTTGTGTTTGGTGAGGACTCCAT |
| | ACTTTGTGAGGGCCCAGGCCCTCATCAGAGTGTGTTCTCTCTTCAAAACGCTAGCTGGGGGACGGTA |
| | CGTGCAGGCCGCGCTGCTCACTATTGGCCGCTGGACCGGCACTTATATTTATAACCATCTCGCCCCC |
| | CTGGAAACATGGGCCGCCGGCGGCCTACGGGATTTGGCCGTTGCAGTCGAGCCCGTGATATTCTCCC |
| | CCATGGAGAAGAAGATCATAGTTTGGGGGGCGGAGACCACTGCTTGTGGCGACATTCTTTGTGGCCT |
| | GCCTGTCTCAGCTCGGCTCGGCAGGGAAGTCCTGCTAGGGCCCGCGGATGACTACAGGTCCATGGGA |
| | TGGCAACTCCTGGCTCCAATCTCAGCATACGCACAGCAGACCCGCGGTCTCATCTCTACCCTTGTCG |
| | TGAGCCTCACTGGCCGTGATAAGAACGAGACTGCCGGCGAGGTGCAGGTGTTGTCCACCTCAACGCA |
| | GACCTTCCTGGGGACCAATGTGGGCGGTGTCATGTGGGGACCTTATCACGGAGCGGGCACGCGGACT |
| | GTGGCCGGCCGGGGTGGGCCTGTCCTCCAGATGTACACGTCAGTCAGTGACGATCTGGTGGGCTGGC |
| | CTGCCCCGCCTGGCTCCAAGTCCCTCGAGCCCTGCTCGTGCGGGTCAGCAGACCTTTACTTGGTGAC |
| | GCGCAATGCTGACGTCCTTCCTCTTAGGAGGAAGGGTGACGGTACAGCGTCCCTACTGTCCCCGCGC |
| | CCTGTGTCCTCCCTTAAAGGCTCCTCGGGAGGGCCTGTCTTGTGCCCCCAAAGTCATTGCGTTGGCA |
| | TCTTCAGAGCTGCTGTCTGCACCCGCGGTGTAGCCAAAGCAGTTCAGTTTGTTCCCATCGAGAAGAT |
| | GCAGGTGGCCCAGAGGTCGCCATCATTCAGTGACAATAGCACCCCTCCAGCGGTGCCCAGCACCTAT |
| | CAAGTAGGCTATTTACATGCCCCCACTGGCAGCGGCAAGAGCACTAAGGTCCCTGCGGCGTATGCCA |
| | GCCAGGGCTACAAAGTGCTCGTGCTTAATCCATCAGTCGCGGCCACCCTTGGGTTTGGTGCCTACAT |
| | GTCCAAGGCTTATGGAATTGATCCAAGTGTGCGCACCGGCGCGCGCACGGTGACCACGGGGGCTCCC |
| | ATAACTTACTCAACCTATGGGAAGTTCCTTGCCGACGGGGGTTGTTCAGGTGGTGCCTATGATATAA |
| | TCATATGTGATGAATGCCATGCCATCGATGCTACCACCGTGGTCGGCATAGGCACCGTGTTGGACCA |
| | GGCAGAGACTAGTGGTGTCCGACTTGTGGTGCTAGCTACGGCCACCCCCCCCGGAAGCGTTACGGTG |
| | CCCCACCCCAACATCGAGGAGGTCGCCCTTGGCAACGATGGGGAAATTCCCTTCTATGGGAAAGCCA |
| | TCCCCCTGCAACACATCAAGGGGGGAAGGCATCTCATTTTCTGTCATTCCAAAAAGAAATGTGATGA |
| | GCTTGCCGGGAAGCTCACCTCATTGGGGCTGACTGCCGTTGCTTACTATAGGGGGTTGGATGTTTCG |
| | GTGATCCCAACGTCTGGCGATGTAGTCGTGGTGGCTACTGATGCCTTGATGACGGGCTTTACCGGAG |
| | ACTTCGACTCCGTCATAGACTGCAACGTGGCGGTTACCCAGACTGTAGACTTCTCCTTGGACCCCAC |
| | CTTCACTATTGAGACCACCACAGTCCCGCAAGATTCTGTCTCGCGCTCTCAGAGACGTGGGAGGACA |
| | GGTAGGGGCAGGCTTGGCATCTACAGATACGTGTCCAGTGGCGAGCGGCCATCCGGTATGTTCGACA |
| | CCAGCGTGCTCTGTGAGTGCTATGACCTGGGGTGCTCGTGGTATGAACTAACGCCCAGTGAAACCAC |
| | CACGCGCCTCAGAGCCTACCTCAATTGCCCTGGTCTTCCAGTGTGCCAAGACCATCTGGAGTTCTGG |
| | GAGGGCGTCTTTACGGGACTTACTCATATTGATGCACACTTCCTGTCCCAGACCAAACAAGAGGGCC |
| | AAAATTATGCGTACCTGACTGCATACCAAGCTACAGTGTGCGCCCGGGCGAAAGCCCCCCCGCCCTC |
| | CTGGGACGTCCAGTGGAAGTGTCTCCAAAGACTAAAGCCACTGTTAGTAGGGCCCACGCCCTTGCTA |
| | TACAGGCTAGGCAGTGTTACCAATGAAGTCACATTCACTCATCCCATCACCAAATACATTGCCACGT |
| | GCATGGCTGCCGACTTGGAGGTCACTACGAGCACCTGGGTGATCGTAGGTGGCGTCCTCGCCGCCGT |
| | GGCCGCATATTGCATGTCTACTGGCAGTGTTGTTGTGGTTGGCCGTGTGGTCCTTGGATCCAACGTG |
| | GTGACGGCCCCCGACAGAGAGGTTTTATACCAGCACTTTGATGAGATGGAAGAGTGCTCCAAAGCTC |
| | CTGAATTGCTGAAACACGCCCAAACCATCGGCGGAATGTTCAAGGACAAGGCCCTCGCCGTCTTGGA |
| | TACGCTCAAGCCGGCCGCCCAGGCTGCTGTTCCCATTGTGGAGACCAACTTCCAAAAGGTGGAGAAG |
| | CTGTGGAATCAGCATATGTGGAACTTCATAAGCGGCATTCAATACCTAGCTGGGCTATCCACACTGC |
| | CAGGTAATCCAACTGTGGCCTCACTGATGGCCTTCACCGCCTCAGTTACTAGCCCCCTTGCTACATC |
| | GACCACACTACTAGTCAACATCCTGGGTGGGTGGTTCGCAAGCCAGTTAGCGCCGCCGTCTGCTGCC |
| | ACGACTTTCGTGGTCTCGGGCCTCGCAGGGGCGGCAGTGGGTAGTGTTGGCTTGGGCAAGGTCTTAG |
| | TTGATGTCCTGGCCGGCTATGGCGCCGGCATAGCTGGCGCCCTGGTGGCCTTCAAAATCATGTCTGG |
| | AGAGGTCCCTAGCACAGAGGATCTGGCAAACCTCTTACCTGCCATACTCTCACCTGGAGCTTTAGTT |
| | GTGGGAGTTGTTTGTGCCGCGATCATCAAACGGCATACCGGCACTAGTGAGGGAGTCACGCAGTGGA |
| | TGAACCGTCTCATTGCGTTCGCCTCCCGTGGAAATCACGTGTCGCCGACCCACTACATACAGGATGA |
| | TGATGCGTCAAAACGCGTCATGGGGATACTCAGCTCCCTTACCATCACCAGCCTCATAAAACGAGTA |
| | CTGGCCTGGGCGCAGACTGAYTATTCTGCGCCCTGTGCCGGGAGCTGGCTCCGGGAGGTGTGGGACT |
| | GGGTTTGCATGGTCCTATCCGACTTTGCATCTTGGTTGAAGGCCAAGGTACTTCCCAGCCTTCCAGG |
| | GATCCCATTCCTATCCTGTCAGAAAGGGTATAAAGGTGAGTGGCGGAACGATGGCATCATGAACACC |
| | AAGTGCCCGTGTGGGGCCCTGATAGCAGGGCACGTCAAGAACGGATCGATGCGCATCGTGGGCCCCA |
| | AGACCTGCCGAAACACCTGGTGGGGAACCTTCCCTATTAATTCCCATACCACGGGACCAAGTTCTCC |
| | TGTACCCTCCCAYTGTTATCAAAGGGCCTTGTGGAGGGTATCTGCRACGGAGTACGTCGAGATCCTT |
| | CGTCACAATGACCAGCATTAYGTGGTGGGAGTGACGGCGGAGGACCTCAAGTGCCCGTGTCAGGTTC |
| | CTTCCCCCGAATTCTTCAGCTTTGTGGACGGGGTGCGGATACACAGATTCGCCCCTGAACCCAAGCC |
| | CATGATCCGGGAAGAAGCCGCTTTCGTGGTGGGTCTGCACTCATATGTGGTGGGGTCTCAGCTCCCT |
| | TGTGAACCGGAGCCGGACGTCCAAACTGTCTCGCAGCTCCTAACCGACCCCTCACACATCACCGCAG |
| | AAACCGCGGCTAGAAGGCTCAGGAGAGGTTCCCCCCCATCCAACGCCAGTTCTTCTGCTAGTCAACT |
| | CTCAGCTCCATCTCTAAAAGCCACCCACACAACTCTGCCACAACATCCTGACGCAGAACTGATAGAG |
| | GCCAACCTCATGTGGGAACATAAGGTTGGTGCCATCAGAAGGATGGAGACAGACACCAAAGTCATCA |
| | TACTTGACTCCTTTGATTCAGCTTCTTCCGTYGAGGATGACATGGAACCGTCCACCGCCGCGGAGTG |
| | TCTGCGTACGCGTAAGGTCTTCCCACCGGCCATGCCCATATGGGCGAGGCCAGACTACAATCCTCCT |
| | GTTGTGGAGAATTGGAAGGACCCCGAGTACGCACCTCCCCAGGTGTCTGGGTGCGCTCTACCACCTG |
| | CGCAGACCCCTCCTGTTCCACCACCCAGGCGGAAGCGGGCGGTTATCCAACTCACAGAGTCCGCGGT |
| | TTCCACGGCACTGGCGGAACTCGCTGAGAGGTCCTTCCCAAAGGAGGAGGCACCCCCCAGCGACTCC |
| | GCCATTAGTCTGGATTCACCGGCGGCAAATGACCCTCCATCTGATTGTGACCAAGGCAGTGAGATCT |
| | CTTTCTCATCCATGCCACCCCTAGAGGGGGAGCCGGGTGATCCAGACTTGTCTGATGGCTCTTGGTC |
| | AACAGTCAGCACCAGATCTGACGTGATTTGCTGTTCCATGTCCTACTCATGGACGGGAGCCTTAGTG |
| | ACGCCCTCCGGTCCGGAGGAGGAAAGGCTCCCAATAAATGCCCTYAGTAACACCATGCTTCGGCATT |
| | ATAACATGGTTTACAGCACAACATCACGCTCAGCTTCACAAAGAGCAAAAAAAGTGACTTTTGACAG |
| | ATTGCAAGTCCTAGACGACCACTACAAAAGGGCGCTCGCCGACGTGAAAGCGGATGCTTCCACGGTT |
| | AAGGCGCAGTTGCTCTCAGTAGAGGAAGCTGCTGCCCTCACCCCTGCCCACTCAGCCAGATCTAAGT |
| | TTGGGTACGGGGCAAAGGAAGTCAGATCTCTTGCGCCCAAGGCAATGTCTCACATCAAAGAGGTCTG |
| | GAAGGACTTGCTACAGGACATGACCACCCCGATACCGACCACCATAATGGCCAAGAATGAAGTCTTC |
| | TGCGTAAATCCTGCCAAGGGCGGGAAAAAGCCAGCTAGACTGATTGTTTACCCTGACCTAGGTGTGA |
| | GGGTATGCGAGAAGCGGGCTTTGTACGACATAGCTCAAAAGCTTCCGAAGGCCATAATGGGGCAGGC |
| | GTACGGGTTCCAATATTCACCATCGCAGCGGGTCGARTACYTAGTTAAGACGTGGAAGTCCAAACGG |
| | ACTCCCATGGGCTTCTCATATGACACCCGCTGCTTTGATTCCACTGTCACAGAACAAGACATCCGCA |
| | CTGAGAGCGAGATCTATCAGTGCTGCAACCTCGACCCCGAGGCTCGCACCATCATTAACGCCCTTAC |
| | CGAGAGGTTGTACGTGGGGGGACCCATGTTCAACTCTAAAGGTCAGAGGGTCGGTTATCGAAGGTGC |
| | AGAGCCAGCGGAGTTTTCCCCACCAGCATGGGAAACACTATGACGTGCTACATCAAAGCCAAAGCGG |
| | CTGCAGCAGCTGCAGGGCTAGAGAGCACTGATTTCCTGGTCTGCGGCGATGACTTGGTGGTCATTTG |
| | CGAGAGCAAGGGAGTCGAACGGGATAGGGCGGACCTGCAAGCCTTTGCAGCGGCAATGACCAGGTAC |
| | TCCGCTCCACCCGGGGACATGCCCCAGCCCGCTTATGACCTTGAGCACATTGACTCTTGCTCATCCA |
| | ATGTCTCAGTCGCCCGGGACAACTCGGGGAAAAGAGTCTACTACCTCACTAGAGACCCTACCAACCC |
| | ACTATCCAGGGCAGCTTGGGAGACCGCCCGTCACTCACCTGTAAACTCCTGGGTGGGCAACATAATC |
| | ATGTTTGCCCCCACCATATGGGTGAGGATGGTTCTCATGACTCACTTTTTCGCTCTCCTCCTCAATG |
| | AGGAGCGATTGAACGATCCAGTGAGTTTTGAGATGTATGGTGCAACCTACACGGTCTGCCCAACAGA |
| | CTTACCAGACATTATACAAAGACTCCATGGGCTCCGCGCATTTGAACTCCATACTTACTCTCCAGCA |
| | GAACTGACGCGGGTGGCAGCTACCCTCAGGAAGCTTGGGGTGCCACCCCTCAGGACCTGGAGACAAC |
| | GGGCTCGCAAGGTCCGCGCGGGGCTGATAGGCCAGGGGGGCAGGGCTCGCATATGCGGTCTCTACCT |
| | CTTCAACTGGGCTGTCAGGACCAAGATCAAACTCACTCCACTTGCAGGCGCTGGCCGGCTCGATCTT |
| | TCGAGCTGGTTCAGCGTTTGCGCCGGCGAGGCGGACGTGGATCACAGCACGCCCAGAGCCCATCCTC |
| | GCCCATTACTCCTGTGCCTACTCCTACTTGCCGTAGGGGTAGGCATTTTCCTCCTACCCGCTCGGTA |
| | AGCGGGTAACACTCCAGGCTTTTAAGCCATAAATTCTGTTTTTTTTTTTTTTTTTTTTTTTTT |
| 290 | GenBank Accession No. MW735442.1 (B.1.1.7 SARS-CoV-2 variant; Severe |
| | acute respiratory syndrome coronavirus 2 isolate SARS-CoV-2/human/PHL/PH- |
| | PGC-03978/2021) |
| | ACTTTCGATCTCTTGTAGATCTGTTCTCTAAACGAACTTTAAAATCTGTGTGGCTGTCACTCGGCTG |
| | CATGCTTAGTGCACTCACGCAGTATAATTAATAACTAATTACTGTCGTTGACAGGACACGAGTAACT |
| | CGTCTATCTTCTGCAGGCTGCTTACGGTTTCGTCCGTGTTGCAGCCGATCATCAGCACATCTAGGTT |
| | TTGTCCGGGTGTGACCGAAAGGTAAGATGGAGAGCCTTGTCCCTGGTTTCAACGAGAAAACACACGT |
| | CCAACTCAGTTTGCCTGTTTTACAGGTTTGCGACGTGCTCGTACGTGGCTTTGGAGACTCCGTGGAG |
| | GAGGTCTTATCAGAGGCACGTCAACATCTTAAAGATGGCACTTGTGGCTTAGTAGAAGTTGAAAAAG |
| | GCGTTTTGCCTCAACTTGAACAGCCCTATGTGTTCATCAAACGTTCGGATGCTCGAACTGCACCTCA |
| | TGGTCATGTTATGGTTGAGCTGGTAGCAGAACTCGAAGGCATTCAGTACGGTCGTAGTGGTGAGACA |
| | CTTGGTGTCCTTGTCCCTCATGTGGGCGAAATACCAGTGGCTTACCGCAAGGTTCTTCTTCGTAAGA |
| | ACGGTAATAAAGGAGCTGGTGGCCATAGTTACGGCGCCGATCTAAAGTCATTTGACTTAGGCGACGA |
| | GCTTGGCACTGATCCTTATGAAGATTTTCAAGAAAACTGGAACACTAAACATAGCAGTGGTGTTACC |
| | CGTGAACTCATGCGTGAGCTTAACGGAGGGGCATACACTCGCTATGTCGATAACAACTTCTGTGGCC |
| | CTGATGGCTACCCTCTTGAGTGCATTAAAGACCTTCTAGCACGTGCTGGTAAAGCTTCATGCACTTT |
| | GTCTGAACAACTGGACTTTATTGACACTAAGAGGGGTGTATACTGCTGCCGTGAACATGAGCATGAA |
| | ATTGCTTGGTACACGGAACGTTCTGAAAAGAGCTATGAATTGCAGACACCTTTTGAAATTAAATTGG |
| | CAAAGAAATTTGAGACCTTCAATGGGGAATGTCCAAATTTTGTATTTCCCTTAAATTCCATAATCAA |
| | GACTATTCAACCAAGGGTTGAAAAGAAAAAGCTTGATGGCTTTATGGGTAGAATTCGATCTGTCTAT |
| | CCAGTTGCGTCACCAAATGAATGCAACCAAATGTGCCTTTCAACTCTCATGAAGTGTGATCATTGTG |
| | GTGAAACTTCATGGCAGACGGGCGATTTTGTTAAAGCCACTTGCGAATTTTGTGGCACTGAGAATTT |
| | GACTAAAGAAGGTGCCACTACTTGTGGTTACTTACCCCAAAATGCTGTTGTTAAAATTTATTGTCCA |
| | GCATGTCACAATTCAGAAGTAGGACCTGAGCATAGTCTTGCCGAATACCATAATGAATCTGGCTTGA |
| | AAACCATTCTTCGTAAGGGTGGTCGCACTATTGCCTTTGGAGGCTGTGTGTTCTCTTATGTTGGTTG |
| | CCATAACAAGTGTGCCTATTGGGTTCCACGTGCTAGCGCTAACATAGGTTGTAACCATACAGGTGTT |
| | GTTGGAGAAGGTTCCGAAGGTCTTAATGACAACCTTCTTGAAATACTCCAAAAAGAGAAAGTCAACA |
| | TCAATATTGTTGGTGACTTTAAACTTAATGAAGAGATCGCCATTATTTTGGCATCTTTTTCTGCTTC |
| | CACAAGTGCTTTTGTGGAAACTGTGAAAGGTTTGGATTATAAAGCATTCAAACAAATTGTTGAATCC |
| | TGTGGTAATTTTAAAGTTACAAAAGGAAAAGCTAAAAAAGGTGCCTGGAATATTGGTGAACAGAAAT |
| | CAATACTGAGTCCTCTTTATGCATTTGCATCAGAGGCTGCTCGTGTTGTACGATCAATTTTCTCCCG |
| | CACTCTTGAAACTGCTCAAAATTCTGTGCGTGTTTTACAGAAGGCCGCTATAACAATACTAGATGGA |
| | ATTTCACAGTATTCACTGAGACTCATTGATGCTATGATGTTCACATCTGATTTGGCTACTAACAATC |
| | TAGTTGTAATGGCCTACATTACAGGTGGTGTTGTTCAGTTGACTTCGCAGTGGCTAACTAACATCTT |
| | TGGCACTGTTTATGAAAAACTCAAACCCGTCCTTGATTGGCTTGAAGAGAAGTTTAAGGAAGGTGTA |
| | GAGTTTCTTAGAGACGGTTGGGAAATTGTTAAATTTATCTCAACCTGTGCTTGTGAAATTGTCGGTG |
| | GACAAATTGTCACCTGTGCAAAGGAAATTAAGGAGAGTGTTCAGACATTCTTTAAGCTTGTAAATAA |
| | ATTTTTGGCTTTGTGTGCTGACTCTATCATTATTGGTGGAGCTAAACTTAAAGCCTTGAATTTAGGT |
| | GAAACATTTGTCACGCACTCAAAGGGATTGTACAGAAAGTGTGTTAAATCCAGAGAAGAAACTGGCC |
| | TACTCATGCCTCTAAAAGCCCCAAAAGAAATTATCTTCTTAGAGGGAGAAACACTTCCCACAGAAGT |
| | GTTAACAGAGGAAGTTGTCTTGAAAACTGGTGATTTACAACCATTAGAACAACCTACTAGTGAAGCT |
| | GTTGAAGCTCCATTGGTTGGTACACCAGTTTGTATTAACGGGCTTATGTTGCTCGAAATCAAAGACA |
| | CAGAAAAGTACTGTGCCCTTGCACCTAATATGATGGTAACAAACAATACCTTCACACTCAAAGGCGG |
| | TGCACCAACAAAGGTTACTTTTGGTGATGACACTGTGATAGAAGTGCAAGGTTACAAGAGTGTGAAT |
| | ATCACTTTTGAACTTGATGAAAGGATTGATAAAGTACTTAATGAGAAGTGCTCTGCCTATACAGTTG |
| | AACTCGGTACAGAAGTAAATGAGTTCGCCTGTGTTGTGGCAGATGCTGTCATAAAAACTTTGCAACC |
| | AGTATCTGAATTACTTACACCACTGGGCATTGATTTAGATGAGTGGAGTATGGCTACATACTACTTA |
| | TTTGATGAGTCTGGTGAGTTTAAATTGGCTTCACATATGTATTGTTCTTTTTACCCTCCAGATGAGG |
| | ATGAAGAAGAAGGTGATTGTGAAGAAGAAGAGTTTGAGCCATCAACTCAATATGAGTATGGTACTGA |
| | AGATGATTACCAAGGTAAACCTTTGGAATTTGGTGCCACTTCTGCTGCTCTTCAACCTGAAGAAGAG |
| | CAAGAAGAAGATTGGTTAGATGATGATAGTCAACAAACTGTTGGTCAACAAGACGGCAGTGAGGACA |
| | ATCAGACAACTATTATTCAAACAATTGTTGAGGTTCAACCTCAATTAGAGATGGAACTTACACCAGT |
| | TGTTCAGACTATTGAAGTGAATAGTTTTAGTGGTTATTTAAAACTTACTGAGAATGTATACATTAAA |
| | AATGCAGACATTGTGGAAGAAGCTAAAAAGGTAAAACCAACAGTGGTTGTTAATGCAGCCAATGTTT |
| | ACCTTAAACATGGAGGAGGTGTTGCAGGAGCCTTAAATAAGGCTACTAACAATGCCATGCAAGTTGA |
| | ATCTGATGATTACATAGCTACTAATGGACCACTTAAAGTGGGTGGTAGTTGTGTTTTAAGCGGACAC |
| | AATCTTGCTAAACACTGTCTTCATGTTGTCGGCCCAAATGTTAACAAAGGTGAAGACATTCAACTTC |
| | TTAAGAGTGCTTATGAAAATTTTAATCAGCACGAAGTTCTACTTGCACCATTATTATCAGCTGGTAT |
| | TTTTGGTGCTGACCCTATACATTCTTTAAGAGTTTGTGTAGATACTGTTCGCACAAATGTCTACTTA |
| | GCTGTCTTTGATAAAAATCTCTATGACAAACTTGTTTCAAGCTTTTTGGAAATGAAGAGTGAAAAGC |
| | AAGTTGAACAAAAGATCGCTGAGATTCCTAAAGAGGAAGTTAAGCCATTTATAACTGAAAGTAAACC |
| | TTCAGTTGAACAGAGAAAACAAGATGATAAGAAAATCAAAGCTTGTGTTGAAGAAGTTACAACAACT |
| | CTGGAAGAAACTAAGTTCCTCACAGAAAACTTGTTACTTTATATTGACATTAATGGCAATCTTCATC |
| | CAGATTCTGCCACTCTTGTTAGTGACATTGACATCACTTTCTTAAAGAAAGATGCTCCATATATAGT |
| | GGGTGATGTTGTTCAAGAGGGTGTTTTAACTGCTGTGGTTATACCTACTAAAAAGGCTGGTGGCACT |
| | ACTGAAATGCTAGCGAAAGCTTTGAGAAAAGTGCCAACAGACAATTATATAACCACTTACCCGGGTC |
| | AGGGTTTAAATGGTTACACTGTAGAGGAGGCAAAGACAGTGCTTAAAAAGTGTAAAAGTGCCTTTTA |
| | CATTCTACCATCTATTATCTCTAATGAGAAGCAAGAAATTCTTGGAACTGTTTCTTGGAATTTGCGA |
| | GAAATGCTTGCACATGCAGAAGAAACACGCAAATTAATGCCTGTCTGTGTGGAAACTAAAGCCATAG |
| | TTTCAACTATACAGCGTAAATATAAGGGTATTAAAATACAAGAGGGTGTGGTTGATTATGGTGCTAG |
| | ATTTTACTTTTACACCAGTAAAACAACTGTAGCGTCACTTATCAACACACTTAACGATCTAAATGAA |
| | ACTCTTGTTACAATGCCACTTGGCTATGTAACACATGGCTTAAATTTGGAAGAAGCTGCTCGGTATA |
| | TGAGATCTCTCAAAGTGCCAGCTACAGTTTCTGTTTCTTCACCTGATGCTGTTACAGCGTATAATGG |
| | TTATCTTACTTCTTCTTCTAAAACACCTGAAGAACATTTTATTGAAACCATCTCACTTGCTGGTTCC |
| | TATAAAGATTGGTCCTATTCTGGACAATCTACACAACTAGGTATAGAATTTCTTAAGAGAGGTGATA |
| | AAAGTGTATATTACACTAGTAATCCTACCACATTCCACCTAGATGGTGAAGTTATCACCTTTGACAA |
| | TCTTAAGACACTTCTTTCTTTGAGAGAAGTGAGGGCTATTAAGGTGTTTACAACAGTAGACAACATT |
| | AACCTCCACACGCANNNNNNNNNNNNNNNNNNNNNNNNNNNNNNNNNNNNNNNNNNNNNNNNNNNNN |
| | NNNNNNNNNNNNNNNNNNNNNNNNNNNNNNNNNNNNNNNNNNNNNNNNNNNNNNNNNNNNNNNNNNN |
| | NNNNNNNNNNNNNNNNNNNNNNNNNNNNNNNNNNNNNNNNNNNNNNNNNNNNNNNNNNNNNNNNNNN |
| | NNNNNNNNNNNNNNNNNNNNNNNNNNNNNNNNNNNNNNNNNNNNNNNNNNNNNNNNNNNNNNNNNNA |
| | TTAAATGGGCAGATAACAACTGTTATCTTGCCACTGCATTGTTAACACTCCAACAAATAGAGTTGAA |
| | GTTTAATCCACCTGCTCTACAAGATGCTTATTACAGAGCAAGGGCTGGTGAAGCTGATAACTTTTGT |
| | GCACTTATCTTAGCCTACTGTAATAAGACAGTAGGTGAGTTAGGTGATGTTAGAGAAACAATGAGTT |
| | ACTTGTTTCAACATGCCAATTTAGATTCTTGCAAAAGAGTCTTGAACGTGGTGTGTAAAACTTGTGG |
| | ACAACAGCAGACAACCCTTAAGGGTGTAGAAGCTGTTATGTACATGGGCACACTTTCTTATGAACAA |
| | TTTAAGAAAGGTGTTCAGATACCTTGTACGTGTGGTAAACAAGCTACAAAATATCTAGTACAACAGG |
| | AGTCACCTTTTGTTATGATGTCAGCACCACCTGCTCAGTATGAACTTAAGCATGGTACATTTACTTG |
| | TGCTAGTGAGTACACTGGTAATTACCAGTGTGGTCACTATAAACATATAACTTCTAAAGAAACTTTG |
| | TATTGCATAGACGGTGCTTTACTTACAAAGTCCTCAGAATACAAAGGTCCTATTACGGATGTTTTCT |
| | ACAAAGAAAACAGTTACACAACAACCATAAAACCAGTTACTTATAAATTGGATGGTGTTGTTTGTAC |
| | AGAAATTGACCCTAAGTTGGACAATTATTATAAGAAAGACAATTCTTATTTTACAGAGCAACCAATT |
| | GATCTTGTACCAAACCAACCATATCCAAACGCAAGCTTCGATAATTTTAAGTTTGTATGTGATAATA |
| | TCAAATTTGCTGATGATTTAAACCAGTTAACTGGTTATAAGAAACCTGCTTCAAGAGAGCTTAAAGT |
| | TACATTTTTCCCTGACTTAAATGGTGATGTGGTGGCTATTGATTATAAACACTACACACCCTCTTTT |
| | AAGAAAGGAGCTAAATTGTTACATAAACCTATTGTTTGGCATGTTAACAATGCAACTAATAAAGCCA |
| | CGTATAAACCAAATACCTGGTGTATACGTTGTCTTTGGAGCACAAAACCAGTTGAAACATCAAATTC |
| | GTTTGATGTACTGAAGTCAGAGGACGCGCAGGGAATGGATAATCTTGCCTGCGAAGATCTAAAACCA |
| | GTCTCTGAAGAAGTAGTGGAAAATCCTACCATACAGAAAGACGTTCTTGAGTGTAATGTGAAAACTA |
| | CCGAAGTTGTAGGAGACATTATACTTAAACCAGCAAATAATAGTTTAAAAATTACAGAAGAGGTTGG |
| | CCACACAGATCTAATGGCTGCTTATGTAGACAATTCTAGTCTTACTATTAAGAAACCTAATGAATTA |
| | TCTAGAGTATTAGGTTTGAAAACCCTTGCTACTCATGGTTTAGCTGCTGTTAATAGTGTCCCTTGGG |
| | ATACTATAGCTAATTATGCTAAGCCTTTTCTTAACAAAGTTGTTAGTACAACTACTAACATAGTTAC |
| | ACGGTGTTTAAACCGTGTTTGTACTAATTATATGCCTTATTTCTTTACTTTATTGCTACAATTGTGT |
| | ACTTTTACTAGAAGTACAAATTCTAGAATTAAAGCATCTATGCCGACTACTATAGCAAAGAATACTG |
| | TTAAGAGTGTCGGTAAATTTTGTCTAGAGGCTTCATTTAATTATTTGAAGTCACCTAATTTTTCTAA |
| | ACTGATAAATATTACAATTTGGTTTTTACTATTAAGTGTTTGCCTAGGTTCTTTAATCTACTCAACC |
| | GCTGCTTTAGGTGTTTTAATGTCTAATTTAGGCATGCCTTCTTACTGTACTGGTTACAGAGAAGGCT |
| | ATTTGAACTCTACTAATGTCACTATTGCAACCTACTGTACTGGTTCTATACCTTGTAGTGTTTGTCT |
| | TAGTGGTTTAGATTCTTTAGACACCTATCCTTCTTTAGAAACTATACAAATTACCATTTCATCTTTT |
| | AAATGGGATTTAACTGTTTTTGGCTTAGTTGCAGAGTGGTTTTTGGCATATATTCTTTTCACTAGGT |
| | TTTTCTATGTACTTGGATTGGCTGCAATCATGCAATTGTTTTTCAGCTATTTTGCAGTACATTTTAT |
| | TAGTAATTCTTGGCTTATGTGGTTAATAATTAATCTTGTACAAATGGCCCCGATTTCAGCTATGGTT |
| | AGAATGTACATCTTCTTTGCATCATTTTATTATGTATGGAAAAGTTATGTGCATGTTGTAGACGGTT |
| | GTAATTCATCAACTTGTATGATGTGTTACAAACGTAATAGAGCAACAAGAGTCGAATGTACAACTAT |
| | TGTTAATGGTGTTAGAAGGTCCTTTTATGTCTATGCTAATGGAGGTAAAGGCTTTTGCAAACTACAC |
| | AATTGGAATTGTGTTAATTGTGATACATTCTGTGCTGGTAGTACATTTATTAGTGATGAAGTTGCGA |
| | GAGACTTGTCACTACAGTTTAAAAGACCAATAAATCCTACTGACCAGTCTTCTTACATCGTTGATAG |
| | TGTTACAGTGAAGAATGGTTCCATCCATCTTTACTTTGATAAAGCTGGTCAAAAGACTTATGAAAGA |
| | CATTCTCTCTCTCATTTTGTTAACTTAGACAACCTGAGAGCTAATAACACTAAAGGTTCATTGCCTA |
| | TTAATGTTATAGTTTTTGATGGTAAATCAAAATGTGAAGAATCATCTGCAAAATCAGCGTCTGTTTA |
| | CTACAGTCAGCTTATGTGTCAACCTATACTGTTACTAGATCAGGCATTAGTGTCTGATGTTGGTGAT |
| | AGTGCGGAAGTTGCAGTTAAAATGTTTGATGCTTACGTTAATACGTTTTCATCAACTTTTAACGTAC |
| | CAATGGAAAAACTCAAAACACTAGTTGCAACTGCAGAAGCTGAACTTGCAAAGAATGTGTCCTTAGA |
| | CAATGTCTTATCTACTTTTATTTCAGCAGCTCGGCAAGGGTTTGTTGATTCAGATGTAGAAACTAAA |
| | GATGTTGTTGAATGTCTTAAATTGTCACATCAATCTGACATAGAAGTTACTGGCGATAGTTGTAATA |
| | ACTATATGCTCACCTATAACAAAGTTGAAAACATGACACCCCGTGACCTTGGTGCTTGTATTGACTG |
| | TAGTGCGCGTCATATTAATGCGCAGGTAGCAAAAAGTCACAACATTGCTTTGATATGGAACGTTAAA |
| | GATTTCATGTCATTGTCTGAACAACTACGAAAACAAATACGTAGTGCTGCTAAAAAGAATAACTTAC |
| | CTTTTAAGTTGACATGTGCAACTACTAGACAAGTTGTTAATGTTGTAACAACAAAGATAGCACTTAA |
| | GGGTGGTAAAATTGTTAATAATTGGTTGAAGCAGTTAATTAAAGTTACACTTGTGTTCCTTTTTGTT |
| | GCTGCTATTTTCTATTTAATAACACCTGTTCATGTCATGTCTAAACATACTGACTTTTCAAGTGAAA |
| | TCATAGGATACAAGGCTATTGATGGTGGTGTCACTCGTGACATAGCATCTACAGATACTTGTTTTGC |
| | TAACAAACATGCTGATTTTGACACATGGTTTAGCCAGCGTGGTGGTAGTTATACTAATGACAAAGCT |
| | TGCCCATTGATTGCTGCAGTCATAACAAGAGAAGTGGGTTTTGTCGTGCCTGGTTTGCCTGGCACGA |
| | TATTACGCACAACTAATGGTGACTTTTTGCATTTCTTACCTAGAGTTTTTAGTGCAGTTGGTAACAT |
| | CTGTTACACACCATCAAAACTTATAGAGTACACTGACTTTGCAACATCAGCTTGTGTTTTGGCTGCT |
| | GAATGTACAATTTTTAAAGATGCTTCTGGTAAGCCAGTACCATATTGTTATGATACCAATGTACTAG |
| | AAGGTTCTGTTGCTTATGAAAGTTTACGCCCTGACACACGTTATGTGCTCATGGATGGCTCTATTAT |
| | TCAATTTCCTAACACCTACCTTGAAGGTTCTGTTAGAGTGGTAACAACTTTTGATTCTGAGTACTGT |
| | AGGCACGGCACTTGTGAAAGATCAGAAGCTGGTGTTTGTGTATCTACTAGTGGTAGATGGGTACTTA |
| | ACAATGATTATTACAGATCTTTACCAGGAGTTTTCTGTGGTGTAGATGCTGTAAATTTACTTACTAA |
| | TATGTTTACACCACTAATTCAACCTATTGGTGCTTTGGACATATCAGCATCTATAGTAGCTGGTGGT |
| | ATTGTAGCTATCGTAGTAACATGCCTTGCCTACTATTTTATGAGGTTTAGAAGAGCTTTTGGTGAAT |
| | ACAGTCATGTAGTTGCCTTTAATACTTTACTATTCCTTATGTCATTCACTGTACTCTGTTTAACACC |
| | AGTTTACTCATTCTTACCTGGTGTTTATTCTGTTATTTACTTGTACTTGACATTTTATCTTACTAAT |
| | GATGTTTCTTTTTTAGCACATATTCAGTGGATGGTTATGTTCACACCTTTAGTACCTTTCTGGATAA |
| | CAATTGCTTATATCATTTGTATTTCCACAAAGCATTTCTATTGGTTCTTTAGTAATTACCTAAAGAG |
| | ACGTGTAGTCTTTAATGGTGTTTCCTTTAGTACTTTTGAAGAAGCTGCGCTGTGCACCTTTTTGTTA |
| | AATAAAGAAATGTATCTAAAGTTGCGTAGTGATGTGCTATTACCTCTTACGCAATATAATAGATACT |
| | TAGCTCTTTATAATAAGTACAAGTATTTTAGTGGAGCAATGGATACAACTAGCTACAGAGAAGCTGC |
| | TTGTTGTCATCTCGCAAAGGCTCTCAATGACTTCAGTAACTCAGGTTCTGATGTTCTTTACCAACCA |
| | CCACAAACCTCTATCACCTCAGCTGTTTTGCAGAGTGGTTTTAGAAAAATGGCATTCCCATCTGGTA |
| | AAGTTGAGGGTTGTATGGTACAAGTAACTTGTGGTACAACTACACTTAACGGTCTTTGGCTTGATGA |
| | CGTAGTTTACTGTCCAAGACATGTGATCTGCACCTCTGAAGACATGCTTAACCCTAATTATGAAGAT |
| | TTACTCATTCGTAAGTCTAATCATAATTTCTTGGTACAGGCTGGTAATGTTCAACTCAGGGTTATTG |
| | GACATTCTATGCAAAATTGTGTACTTAAGCTTAAGGTTGATACAGCCAATCCTAAGACACCTAAGTA |
| | TAAGTTTGTTCGCATTCAACCAGGACAGACTTTTTCAGTGTTAGCTTGTTACAATGGTTCACCATCT |
| | GGTGTTTACCAATGTGCTATGAGGCCCAATTTCACTATTAAGGGTTCATTCCTTAATGGTTCATGTG |
| | GTAGTGTTGGTTTTAACATAGATTATGACTGTGTCTCTTTTTGTTACATGCACCATATGGAATTACC |
| | AACTGGAGTTCATGCTGGCACAGACTTAGAAGGTAACTTTTATGGACCTTTTGTTGACAGGCAAACA |
| | GCACAAGCAGCTGGTACGGACACAACTATTACAGTTAATGTTTTAGCTTGGTTGTACGCTGCTGTTA |
| | TAAATGGAGACAGGTGGTTTCTCAATCGATTTACCACAACTCTTAATGACTTTAACCTTGTGGCTAT |
| | GAAGTACAATTATGAACCTCTAACACAAGACCATGTTGACATACTAGGACCTCTTTCTGCTCAAACT |
| | GGAATTGCCGTTTTAGATATGTGTGCTTCATTAAAAGAATTACTGCAAAATGGTATGAATGGACGTA |
| | CCATATTGGGTAGTGCTTTATTAGAAGATGAATTTACACCTTTTGATGTTGTTAGACAATGCTCAGG |
| | TGTTACTTTCCAAAGTGCAGTGAAAAGAACAATCAAGGGTACACACCACTGGTTGTTACTCACAATT |
| | TTGACTTCACTTTTAGTTTTAGTCCAGAGTACTCAATGGTCTTTGTTCTTTTTTTTGTATGAAAATG |
| | CCTTTTTACCTTTTGCTATGGGTATTATTGCTATGTCTGCTTTTGCAATGATGTTTGTCAAACATAA |
| | GCATGCATTTCTCTGTTTGTTTTTGTTACCTTCTCTTGCCACTGTAGCTTATTTTAATATGGTCTAT |
| | ATGCCTGCTAGTTGGGTGATGCGTATTATGACATGGTTGGATATGGTTGATACTAGTTTGAAGCTAA |
| | AAGACTGTGTTATGTATGCATCAGCTGTAGTGTTACTAATCCTTATGACAGCAAGAACTGTGTATGA |
| | TGATGGTGCTAGGAGAGTGTGGACACTTATGAATGTCTTGACACTCGTTTATAAAGTTTATTATGGT |
| | AATGCTTTAGATCAAGCCATTTCCATGTGGGCTCTTATAATCTCTGTTACTTCTAACTACTCAGGTG |
| | TAGTTACAACTGTCATGTTTTTGGCCAGAGGTATTGTTTTTATGTGTGTTGAGTATTGCCCTATTTT |
| | CTTCATAACTGGTAATACACTTCAGTGTATAATGCTAGTTTATTGTTTCTTAGGCTATTTTTGTACT |
| | TGTTACTTTGGCCTCTTTTGTTTACTCAACCGCTACTTTAGACTGACTCTTGGTGTTTATGATTACT |
| | TAGTTTCTACACAGGAGTTTAGATATATGAATTCACAGGGACTACTCCCACCCAAGAATAGCATAGA |
| | TGCCTTCAAACTCAACATTAAATTGTTGGGTGTTGGTGGCAAACCTTGTATCAAAGTAGCCACTGTA |
| | CAGTCTAAAATGTCAGATGTAAAGTGCACATCAGTAGTCTTACTCTCAGTTTTGCAACAACTCAGAG |
| | TAGAATCATCATCTAAATTGTGGGCTCAATGTGTCCAGTTACACAATGACATTCTCTTAGCTAAAGA |
| | TACTACTGAAGCCTTTGAAAAAATGGTTTCACTACTTTCTGTTTTGCTTTCCATGCAGGGTGCTGTA |
| | GACATAAACAAGCTTTGTGAAGAAATGCTGGACAACAGGGCAACCTTACAAGCTATAGCCTCAGAGT |
| | TTAGTTCCCTTCCATCATATGCAGCTTTTGCTACTGCTCAAGAAGCTTATGAGCGGGCTGTTGCTAA |
| | TGGTGATTCTGAAGTTGTTCTTAAAAAGTTGAAGAAGTCTTTGAATGTGGCTAAATCTGAATTTGAC |
| | CGTGATGCAGCCATGCAACGTAAGTTGGAAAAGATGGCTGATCAAGCTATGACCCAAATGTATAAAC |
| | AGGCTAGATCTGAGGACAAGAGGGCAAAAGTTACTAGTGCTATGCAGACAATGCTTTTCACTATGCT |
| | TAGAAAGTTGGATAATGATGCACTCAACAACATTATCAACAATGCAAGAGATGGTTGTGTTCCCTTG |
| | AACATAATACCTCTTACAACAGCAGCCAAACTAATGGTTGTCATACCAGACTATAACACATATAAAA |
| | ATACGTGTGATGGTACAACATTTACTTATGCATCAGCATTGTGGGAAATCCAACAGGTTGTAGATGC |
| | AGATAGTAAAATTGTTCAACTTAGTGAAATTAGTATGGACAATTCACCTAATTTAGCATGGCCTCTT |
| | ATTGTAACAGCTTTAAGGGCCAATTCTGCTGTCAAATTACAGAATAATGAGCTTAGTCCTGTTGCAC |
| | TACGACAGATGTCTTGTGCTGCCGGTACTACACAAACTGCTTGCACTGATGACAATGCGTTAGCTTA |
| | CTACAACACAACAAAGGGAGGTAGGTTTGTACTTGCACTGTTATCCGATTTACAGGATTTGAAATGG |
| | GCTAGATTCCCTAAGAGTGATGGAACTGGTACTATCTATACAGAACTGGAACCACCTTGTAGGTTTG |
| | TTACAGACACACCTAAAGGTCCTAAAGTGAAGTATTTATACTTTATTAAAGGATTAAACAACCTAAA |
| | TAGAGGTATGGTACTTGGTAGTTTAGCTGCCACAGTACGTCTACAAGCTGGTAATGCAACAGAAGTG |
| | CCTGCCAATTCAACTGTATTATCTTTCTGTGCTTTTGCTGTAGATGCTGCTAAAGCTTACAAAGATT |
| | ATCTAGCTAGTGGGGGACAACCAATCACTAATTGTGTTAAGATGTTGTGTACACACACTGGTACTGG |
| | TCAGGCAATAACAGTTACACCGGAAGCCAATATGGATCAAGAATCCTTTGGTGGTGCATCGTGTTGT |
| | CTGTACTGCCGTTGCCACATAGATCATCCAAATCCTAAAGGATTTTGTGACTTAAAAGGTAAGTATG |
| | TACAAATACCTACAACTTGTGCTAATGACCCTGTGGGTTTTACACTTAAAAACACAGTCTGTACCGT |
| | CTGCGGTATGTGGAAAGGTTATGGCTGTAGTTGTGATCAANNNNNNNAACCCATGCTTCAGTCAGCT |
| | GATGCACAATCGTTTTTAAACGGGTTTGCGGTGTAAGTGCAGCCCGTCTTACACCGTGCGGCACAGG |
| | CACTAGTACTGATGTCGTATACAGGGCTTTTGACATCTACAATGATAAAGTAGCTGGTTTTGCTAAA |
| | TTCCTAAAANNNNNNTGTNNTCGCTTCCAAGAAAAGGACGAAGATGACAATTTAATTGATTCTTACT |
| | TTGTAGTTAAGAGACACACTTTCTCTAACTACCAACATGAAGAAACAATTTATAATTTACTTAAGGA |
| | TTGTCCAGCTGTTGCTAAACATGACTTCTTTAAGTTTAGAATAGACGGTGACATGGTACCACATATA |
| | TCACGTCAACGTCTTACTAAATACACAATGGCAGACCTCGTCTATGCTTTAAGGCATTTTGATGAAG |
| | GTAATTGTGACACATTAAAAGAAATACTTGTCACATACAATTGTTGTGATGATGATTATTTCAATAA |
| | AAAGGACTGGTATGATTTTGTAGAAAACCCAGATATATTACGCGTATACGCCAACTTAGGTGAACGT |
| | GTACGCCAAGCTTTGTTAAAAACAGTACAATTCTGTGATGCCATGCGAAATGCTGGTATTGTTGGTG |
| | TACTGACATTAGATAATCAAGATCTCAATGGTAACTGGTATGATTTCGGTGATTTCATACAAACCAC |
| | GCCAGGTAGTGGAGTTCCTGTTGTAGATTCTTATTATTCATTGTTAATGCCTATATTAACCTTGACC |
| | AGGGCTTTAACTGCAGAGTCACATGTTGACACTGACTTAACAAAGCCTTACATTAAGTGGGATTTGT |
| | TAAAATATGACTTCACGGAAGAGAGGTTAAAACTCTTTGACCGTTATTTTAAATATTGGGATCAGAC |
| | ATACCACCCAAATTGTGTTAACTGTTTGGATGACAGATGCATTCTGCATTGTGCAAACTTTAATGTT |
| | TTATTCTCTACAGTGTTCCCACTTACAAGTTTTGGACCACTAGTGAGAAAAATATTTGTTGATGGTG |
| | TTCCATTTGTAGTTTCAACTGGATACCACTTCAGAGAGCTAGGTGTTGTACATAATCAGGATGTAAA |
| | CTTACATAGCTCTAGACTTAGTTTTAAGGAATTACTTGTGTATGCTGCTGACCCTGCTATGCACGCT |
| | GCTTCTGGTAATCTATTACTAGATAAACGCACTACGTGCTTTTCAGTAGCTGCACTTACTAACAATG |
| | TTGCTTTTCAAACTGTCAAACCTGGTAATTTTAACAAAGACTTCTATGACTTTGCTGTGTCTAAGGG |
| | TTTCTTTAAGGAAGGAAGTTCTGTTGAATTAAAACACTTCTTCTTTGCTCAGGATGGTAATGCTGCT |
| | ATCAGCGATTATGACTACTATCGTTATAATCTACCAACAATGTGTGATATCAGACAACTACTATTTG |
| | TAGTTGAAGTTGTTGATAAGTACTTTGATTGTTACGATGGTGGCTGTATTAATGCTAACCAAGTCAT |
| | CGTCAACAACCTAGACAAATCAGCTGGTTTTCCATTTAATAAATGGGGTAAGGCTAGACTTTATTAT |
| | GATTCAATGAGTTATGAGGATCAAGATGCACTTTTCGCATATACAAAACGTAATGTCATCCCTACTA |
| | TAACTCAAATGAATCTTAAGTATGCCATTAGTGCAAAGAATAGAGCTCGCACCGTAGCTGGTGTCTC |
| | TATCTGTAGTACTATGACCAATAGACAGTTTCATCAAAAATTATTGAAATCAATAGCCGCCACTAGA |
| | GGAGCTACTGTAGTAATTGGAACAAGCAAATTCTATGGTGGTTGGCACAACATGTTAAAAACTGTTT |
| | ATAGTGATGTAGAAAACCCTCATCTTATGGGTTGGGATTATCCTAAATGTGATAGAGCCATGCCTAA |
| | CATGCTTAGAATTATGGCCTCACTTGTTCTTGCTCGCAAACATACAACGTGTTGTAGCTTGTCACAC |
| | CGTTTCTATAGATTAGCTAATGAGTGTGCTCAAGTATTGAGTGAAATGGTCATGTGTGGCGGTTCAC |
| | TATATGTTAAACCAGGTGGAACCTCATCAGGAGATGCCACAACTGCTTATGCTAATAGTGTTTTTAA |
| | CATTTGTCAAGCTGTCACGGCCAATGTTAATGCACTTTTATCTACTGATGGTAACAAAATTGCCGAT |
| | AAGTATGTCCGCAATTTACAACACAGACTTTATGAGTGTCTCTATAGAAATAGAGATGTTGACACAG |
| | ACTTTGTGAATGAGTTTTACGCATATTTGCGTAAACATTTCTCAATGATGATACTCTCTGACGATGC |
| | TGTTGTGTGTTTCAATAGCACTTATGCATCTCAAGGTCTAGTGGCTAGCATAAAGAACTTTAAGTCA |
| | GTTCTTTATTATCAAAACAATGTTTTTATGTCTGAAGCAAAATGTTGGACTGAGACTGACCTTACTA |
| | AAGGACCTCATGAATTTTGCTCTCAACATACAATGCTAGTTAAACAGGGTGATGATTATGTGTACCT |
| | TCCTTACCCAGATCCATCAAGAATCCTAGGGGCCGGCTGTTTTGTAGATGATATCGTAAAAACAGAT |
| | GGTACACTTATGATTGAACGGTTCGTGTCTTTAGCTATAGATGCTTACCCACTTACTAAACATCCTA |
| | ATCAGGAGTATGCTGATGTCTTTCATTTGTACTTACAATACATAAGAAAGCTACATGATGAGTTAAC |
| | AGGACACATGTTAGACATGTATTCTGTTATGCTTACTAATGATAACACCTCAAGGTATTGGGAACCT |
| | GAGTTTTATGAGGCTATGTACACACCGCATACAGTCTTACAGGCTGTTGGGGCTTGTGTTCTTTGCA |
| | ATTCACAGACTTCATTAAGATGTGGTGCTTGCATACGTAGACCATTCTTATGTTGTAAATGCTGTTA |
| | CGACCATGTCATATCAACATCACATAAATTAGTCTTGTCTGTTAATCCGTATGTTTGCAATGCTCCA |
| | GGTTGTGATGTCACAGATGTGACTCAACTTTACTTAGGAGGTATGAGCTATTATTGTAAATCACATA |
| | AACCACCCATTAGTTTTCCATTGTGTGCTAATGGACAAGTTTTTGGTTTATATAAAAATACATGTGT |
| | TGGTAGCGATAATGTTACTGACTTTAATGCAATTGCAACATGTGACTGGACAAATGCTGGTGATTAC |
| | ATTTTAGCTAACACCTGTACTGAAAGACTCAAGCTTTTTGCAGCAGAAACGCTCAAAGCTACTGAGG |
| | AGACATTTAAACTGTCTTATGGTATTGCTACTGTACGTGAAGTGCTGTCTGACAGAGAATTACATCT |
| | TTCATGGGAAGTTGGTAAACCTAGACCACCACTTAACCGAAATTATGTCTTTACTGGTTATCGTGTA |
| | ACTAAAAACAGTAAAGTACAAATAGGAGAGTACACCTTTGAAAAAGGTGACTATGGTGATGCTGTTG |
| | TTTACCGAGGTACAACAACTTACAAATTAAATGTTGGTGATTATTTTGTGCTGACATCACATACAGT |
| | AATGCCATTAAGTGCACCTACACTAGTGCCACAAGAGCACTATGTTAGAATTACTGGCTTATACCCA |
| | ACACTCAATATCTCAGATGAGTTTTCTAGCAATGTTGCAAATTATCAAAAGGTTGGTATGCAAAAGT |
| | ATTCTACACTCCAGGGACCACCTGGTACTGGTAAGAGTCATTTTGCTATTGGCCTAGCTCTCTACTA |
| | CCCTTCTGCTCGCATAGTGTATACAGCTTGCTCTCATGCCGCTGTTGATGCACTATGTGAGAAGGCA |
| | TTAAAATATTTGCCTATAGATAAATGTAGTAGAATTATACCTGCACGTGCTCGTGTAGAGTGTTTTG |
| | ATAAATTCAAAGTGAATTCAACATTAGAACAGTATGTCTTTTGTACTGTAAATGCATTGCCTGAGAC |
| | GACAGCAGATATAGTTGTCTTTGATGAAATTTCAATGGCCACAAATTATGATTTGAGTGTTGTCAAT |
| | GCCAGATTACGTGCTAAGCACTATGTGTACATTGGCGACCCTGCTCAATTACCTGCACCACGCACAT |
| | TGCTAACTAAGGGCACACTAGAACCAGAATATTTCAATTCAGTGTGTAGACTTATGAAAACTATAGG |
| | TCCAGACATGTTCCTCGGAACTTGTCGGCGTTGTCCTGCTGAAATTGTTGACACTGTGAGTGCTTTG |
| | GTTTATGATAATAGGCTTAAAGCACATAAAGACAAATCAGCTCAATGCTTTAAAATGTTTTATAAGG |
| | GTGTTATCACGCATGATGTTTCATCTGCAATTAACAGGCCACAAATAGGCGTGGTAAGAGAATTCCT |
| | TACACGTAACCCTGCTTGGAGAAAAGCTGTCTTTATTTCACCTTATAATTCACAGAATGCTGTAGCC |
| | TCAAAGATTTTGGGACTACCAACTCAAACTGTTGATTCATCACAGGGCTCAGAATATGACTATGTCA |
| | TATTCACTCAAACCACTGAAACAGCTCACTCTTGTAATGTAAACAGATTTAATGTTGCTATTACCAG |
| | AGCAAAAGTAGGCATACTTTGCATAATGTCTGATAGAGACCTTTATGACAAGTTGCAATTTACAAGT |
| | CTTGAAATTCCACGTAGGAATGTGGCAACTTTACAAGCTGAAAATGTAACAGGACTCTTTAAAGATT |
| | GTAGTAAGGTAATCACTGGGTTACATCCTACACAGGCACCTACACACCTCAGTGTTGACACTAAATT |
| | CAAAACTGAAGGTTTATGTGTTGACATACCTGGCATACCTAAGGACATGACCTATAGAAGACTCATC |
| | TCTATGATGGGTTTTAAAATGAATTATCAAGTTAATGGTTACCCTAACATGTTTATCACCCGCGAAG |
| | AAGCTATAAGACATGTACGTGCATGGATTGGCTTCGATGTCGAGGGGTGTCATGCTACTAGAGAAGC |
| | TGTTGGTACCAATTTACCTTTACAGCTAGGTTTTTCTACAGGTGTTAACCTAGTTGCTGTACCTACA |
| | GGTTATGTTGATACACCTAATAATACAGATTTTTCCAGAGTTAGTGCTAAACCACCGCCTGGAGATC |
| | AATTTAAACACCTCATACCACTTATCTACAAACCACTTCCTTCCAATCTACTCCCTATAAACATTCT |
| | ACAAATGTTAAGTGACACACTTAAAAATCTCTCTGACAGAGTCGTATTTGTCTTATGGGCACATGGC |
| | TTTGAGTTGACATCTATGAAGTATTTTGTGAAAATAGGACCTGAGCGCACCTGTTGTCTATGTGATA |
| | GACGTGCCACATGCTTTTCCACTGCTTCAGACACTTATGCCTGTTGGCATCATTCTATTGGATTTGA |
| | TTACGTCTATAATCCGTTTATGATTGATGTTCAACAATGGGGTTTTACAGGTAACCTACAAAGCAAC |
| | CATGATCTGTATTGTCAAGTCCATGGTAATGCACATGTAGCTAGTTGTGATGCAATCATGACTAGGT |
| | GTCTAGCTGTCCACGAGTGCTTTGTTAAGCGTGTTGACTGGACTATTGAATATCCTATAATTGGTGA |
| | TGAACTGAAGATTAATGCGGTTTGTAGAAAGGTTCAACACATGGTTGTTAAAGCTGCATTATTAGCA |
| | GACAAATTCCCAGTTCTTCACGACATTGGTAACCCTAAAGCTATTAAGTGTGTACCTCAAGCTGATG |
| | TAGAATGGAAGTTCTATGATGCACAGCCTTGTAGTGACAAAGCTTATAAAATAGAAGAATTATTCTA |
| | TTCTTATGCCACACATTCTGACAAATTCACAGATGGTGTATGCCTATTTTGGAATTGCAATGTCGAT |
| | AGATATCCTGCTAATTCCATTGTTTGTAGATTTGACACTAGAGTGCTATCTAACCTTAACTTGCCTG |
| | GTTGTGATGGTGGCAGTTTGTATGTAAATAAACATGCATTCCACACACCAGCTTTTGATAAAAGTGC |
| | TTTTGTTAATTTAAAACAATTACCATTTTTCTATTACTCTGACAGTCCATGTGAGTCTCATGGAAAA |
| | CAAGTAGTGTCAGATATAGATTATGTACCACTAAAGTCTGCTACGTGTATAACACGTTGCAATTTAG |
| | GTGGTGCTGTCTGTAGACATCATGCTAATGAGTACAGATTGTATCTCGATGCTTATAACATGATGAT |
| | CTCAGCTGGCTTTAGCTTGTGGGTTTACAAACAATTTGATACTTATAACCTCTGGAACACTTTTACA |
| | AGACTTCAGAGTTTAGAAAATGTGGCTTTTAATGTTGTAAATAAGGGACACTTTGATGGACAACAGG |
| | GTGAAGTACCAGTTTCTATCATTAATAACACTGTTTACACAAAAGTTGATGGTGTTGATGTAGAATT |
| | GTTTGAAAATAAAACAACATTACCTGTTAATGTAGCATTTGAGCTTTGGGCTAAGCGCAACATTAAA |
| | CCAGTACCAGAGGTGAAAATACTCAATAATTTGGGTGTGGACATTGCTGCTAATACTGTGATCTGGG |
| | ACTACAAAAGAGATGCTCCAGCACATATATCTACTATTGGTGTTTGTTCTATGACTGACATAGCCAA |
| | GAAACCAACTGAAACGATTTGTGCACCACTCACTGTCTTTTTTGATGGTAGAGTTGATGGTCAAGTA |
| | GACTTATTTAGAAATGCCCGTAATGGTGTTCTTATTACAGAAGGTAGTGTTAAAGGTTTACAACCAT |
| | CTGTAGGTCCCAAACAAGCTAGTCTTAATGGAGTCACATTAATTGGAGAAGCCGTAAAAACACAGTT |
| | CAATTATTATAAGAAAGTTGATGGTGTTGTCCAACAATTACCTGAAACTTACTTTACTCAGAGTAGA |
| | AATTTACAAGAATTTAAACCCAGGAGTCAAATGGAAATTGATTTCTTAGAATTAGCTATGGATGAAT |
| | TCATTGAACGGTATAAATTAGAAGGCTATGCCTTCGAACATATCGTTTATGGAGATTTTAGTCATAG |
| | TCAGTTAGGTGGTTTACATCTACTGATTGGACTAGCTAAACGTTTTAAGGAATCACCTTTTGAATTA |
| | GAAGATTTTATTCCTATGGACAGTACAGTTAAAAACTATTTCATAACAGATGCGCAAACAGGTTCAT |
| | CTAAGTGTGTGTGTTCTGTTATTGATTTATTACTTGATGATTTTGTTGAAATAATAAAATCCCAAGA |
| | TTTATCTGTAGTTTCTAAGGTTGTCAAAGTGACTATTGACTATACAGAAATTTCATTTATGCTTTGG |
| | TGTAAAGATGGCCATGTAGAAACATTTTACCCAAAATTACAATCTAGTCAAGCGTGGCAACCGGGTG |
| | TTGCTATGCCTAATCTTTACAAAATGCAAAGAATGCTATTAGAAAAGTGTGACCTTCAAAATTATGG |
| | TGATAGTGCAACATTACCTAAAGGCATAATGATGAATGTCGCAAAATATACTCAACTGTGTCAATAT |
| | TTAAACACATTAACATTAGCTGTACCCTATAATATGAGAGTTATACATTTTGGTGCTGGTTCTGATA |
| | AAGGAGTTGCACCAGGTACAGCTGTTTTAAGACAGTGGTTGCCTACGGGTACGCTGCTTGTCGATTC |
| | AGATCTTAATGACTTTGTCTCTGATGCAGATTCAACTTTGATTGGTGATTGTGCAACTGTACATACA |
| | GCTAATAAATGGGATCTCATTATTAGTGATATGTACGACCCTAAGACTAAAAATGTTACAAAAGAAA |
| | ATGACTCTAAAGAGGGTTTTTTCACTTACATTTGTGGGTTTATACAACAAAAGCTAGCTCTTGGAGG |
| | TTCCGTGGCTATAAAGATAACAGAACATTCTTGGAATGCTGATCTTTATAAGCTCATGGGACACTTC |
| | GCATGGTGGACAGCCTTTGTTACTAATGTGAATGCGTCATCATCTGAAGCATTTTTAATTGGATGTA |
| | ATTATCTTGGCAAACCACGCGAACAAATAGATGGTTATGTCATGCATGCAAATTACATATTTTGGAG |
| | GAATACAAATCCAATTCAGTTGTCTTCCTATTCTTTATTTGACATGAGTAAATTTCCCCTTAAATTA |
| | AGGGGTACTGCTGTTATGTCTTTAAAAGAAGGTCAAATCAATGATATGATTTTATCTCTTCTTAGTA |
| | AAGGTAGACTTATAATTAGAGAAAACAACAGAGTTGTTATTTCTAGTGATGTTCTTGTTAACAACTA |
| | AACGAACAATGTTTGTTTTTCTTGTTTTATTGCCACTAGTCTCTAGTCAGTGTGTTAATCTTACAAC |
| | CAGAACTCAATTACCCCCTGCATACACTAATTCTTTCACACGTGGTGTTTATTACCCTGACAAAGTT |
| | TTCAGATCCTCAGTTTTACATTCAACTCAGGACTTGTTCTTACCTTTCTTTTCCAATGTTACTTGGT |
| | TCCATGCTATCTCTGGGACCAATGGTACTAAGAGGTTTGATAACCCTGTCCTACCATTTAATGATGG |
| | TGTTTATTTTGCTTCCACTGAGAAGTCTAACATAATAAGAGGCTGGATTTTTGGTACTACTTTAGAT |
| | TCGAAGACCCAGTCCCTACTTATTGTTAATAACGCTACTAATGTTGTTATTAAAGTCTGTGAATTTC |
| | AATTTTGTAATGATCCATTTTTGGGTGTTTACCACAAAAACAACAAAAGTTGGATGGAAAGTGAGTT |
| | CAGAGTTTATTCTAGTGCGAATAATTGCACTTTTGAATATGTCTCTCAGCCTTTTCTTATGGACCTT |
| | GAAGGAAAACAGGGTAATTTCAAAAATCTTAGGGAATTTGTGTTTAAGAATATTGATGGTTATTTTA |
| | AAATATATTCTAAGCACACGCCTATTAATTTAGTGCGTGATCTCCCTCAGGGTTTTTCGGCTTTAGA |
| | ACCATTGGTAGATTTGCCAATAGGTATTAACATCACTAGGTTTCAAACTTTACTTGCTTTACATAGA |
| | AGTTATTTGACTCCTGGTGATTCTTCTTCAGGTTGGACAGCTGGTGCTGCAGCTTATTATGTGGGTT |
| | ATCTTCAACCTAGGACTTTTCTATTAAAATATAATGAAAATGGAACCATTACAGATGCTGTAGACTG |
| | TGCACTTGACCCTCTCTCAGAAACAAAGTGTACGTTGAAATCCTTCACTGTAGAAAAAGGAATCTAT |
| | CAAACTTCTAACTTTAGAGTCCAACCAACAGAATCTATTGTTAGATTTCCTAATATTACAAACTTGT |
| | GCCCTTTTGGTGAAGTTTTTAACGCCACCAGATTTGCATCTGTTTATGCTTGGAACAGGAAGAGAAT |
| | CAGCAACTGTGTTGCTGATTATTCTGTCCTATATAATTCCGCATCATTTTCCACTTTTAAGTGTTAT |
| | GGAGTGTCTCCTACTAAATTAAATGATCTCTGCTTTACTAATGTCTATGCAGATTCATTTGTAATTA |
| | GAGGTGATGAAGTCAGACAAATCGCTCCAGGGCAAACTGGAAAGATTGCTGATTATAATTATAAATT |
| | ACCAGATGATTTTACAGGCTGCGTTATAGCTTGGAATTCTAACAATCTTGATTCTAAGGTTGGTGGT |
| | AATTATAATTACCTGTATAGATTGTTTAGGAAGTCTAATCTCAAACCTTTTGAGAGAGATATTTCAA |
| | CTGAAATCTATCAGGCCGGTAGCACACCTTGTAATGGTGTTGAAGGTTTTAATTGTTACTTTCCTTT |
| | ACAATCATATGGTTTCCAACCCACTTATGGTGTTGGTTACCAACCATACAGAGTAGTAGTACTTTCT |
| | TTTGAACTTCTACATGCACCAGCAACTGTTTGTGGACCTAAAAAGTCTACTAATTTGGTTAAAAACA |
| | AATGTGTCAATTTCAACTTCAATGGTTTAACAGGCACAGGTGTTCTTACTGAGTCTAACAAAAAGTT |
| | TCTGCCTTTCCAACAATTTGGCAGAGACATTGATGACACTACTGATGCTGTCCGTGATCCACAGACA |
| | CTTGAGATTCTTGACATTACACCATGTTCTTTTGGTGGTGTCAGTGTTATAACACCAGGAACAAATA |
| | CTTCTAACCAGGTTGCTGTTCTTTATCAGGGTGTTAACTGCACAGAAGTCCCTGTTGCTATTCATGC |
| | AGATCAACTTACTCCTACTTGGCGTGTTTATTCTACAGGTTCTAATGTTTTTCAAACACGTGCAGGC |
| | TGTTTAATAGGGGCTGAACATGTCAACAACTCATATGAGTGTGACATACCCATTGGTGCAGGTATAT |
| | GCGCTAGTTATCAGACTCAGACTAATTCTCGTCGGCGGGCACGTAGTGTAGCTAGTCAATCCATCAT |
| | TGCCTACACTATGTCACTTGGTGCAGAAAATTCAGTTGCTTACTCTAATAACTCTATTGCCATACCC |
| | ATAAATTTTACTATTAGTGTTACCACAGAAATTCTACCAGTGTCTATGACCAAGACATCAGTAGATT |
| | GTACAATGTACATTTGTGGTGATTCAACTGAATGCAGCAATCTTTTGTTGCAATATGGCAGTTTTTG |
| | TACACAATTAAACCGTGCTTTAACTGGAATAGCTGTTGAACAAGACAAAAACACCCAAGAAGTTTTT |
| | GCACAAGTCAAACAAATTTACAAAACACCACCAATTAAAGATTTTGGTGGTTTTAATTTTTCACAAA |
| | TATTACCAGATCCATCAAAACCAAGCAAGAGGTCATTTATTGAAGATCTACTTTTCAACAAAGTGAC |
| | ACTTGCAGATGCTGGCTTCATCAAACAATATGGTGATTGCCTTGGTGATATTGCTGCTAGAGACCTC |
| | ATTTGTGCACAAAAGTTTAACGGCCTTACTGTTTTGCCACCTTTGCTCACAGATGAAATGATTGCTC |
| | AATACACTTCTGCACTGTTAGCGGGTACAATCACTTCTGGTTGGACCTTTGGTGCAGGTGCTGCATT |
| | ACAAATACCATTTGCTATGCAAATGGCTTATAGGTTTAATGGTATTGGAGTTACACAGAATGTTCTC |
| | TATGAGAACCAAAAATTGATTGCCAACCAATTTAATAGTGCTATTGGCAAAATTCAAGACTCACTTT |
| | CTTCCACAGCAAGTGCACTTGGAAAACTTCAAGATGTGGTCAACCAAAATGCACAAGCTTTAAACAC |
| | GCTTGTTAAACAACTTAGCTCCAATTTTGGTGCAATTTCAAGTGTTTTAAATGATATCCTTGCACGT |
| | CTTGACAAAGTTGAGGCTGAAGTGCAAATTGATAGGTTGATCACAGGCAGACTTCAAAGTTTGCAGA |
| | CATATGTGACTCAACAATTAATTAGAGCTGCAGAAATCAGAGCTTCTGCTAATCTTGCTGCTACTAA |
| | AATGTCAGAGTGTGTACTTGGACAATCAAAAAGAGTTGATTTTTGTGGAAAGGGCTATCATCTTATG |
| | TCCTTCCCTCAGTCAGCACCTCATGGTGTAGTCTTCTTGCATGTGACTTATGTCCCTGCACAAGAAA |
| | AGAACTTCACAACTGCTCCTGCCATTTGTCATGATGGAAAAGCACACTTTCCTCGTGAAGGTGTCTT |
| | TGTTTCAAATGGCACACACTGGTTTGTAACACAAAGGAATTTTTATGAACCACAAATCATTACTACA |
| | CACAACACATTTGTGTCTGGTAACTGTGATGTTGTAATAGGAATTGTCAACAACACAGTTTATGATC |
| | CTTTGCAACCTGAATTAGACTCATTCAAGGAGGAGTTAGATAAATATTTTAAGAATCATACATCACC |
| | AGATGTTGATTTAGGTGACATCTCTGGCATTAATGCTTCAGTTGTAAACATTCAAAAAGAAATTGAC |
| | CGCCTCAATGAGGTTGCCAAGAATTTAAATGAATCTCTCATCGATCTCCAAGAACTTGGAAAGTATG |
| | AGCAGTATATAAAATGGCCATGGTACATTTGGCTAGGTTTTATAGCTGGCTTGATTGCCATAGTAAT |
| | GGTGACAATTATGCTTTGCTGTATGACCAGTTGCTGTAGTTGTCTCAAGGGCTGTTGTTCTTGTGGA |
| | TCCTGCTGCAAATTTGATGAAGACGACTCTGAGCCAGTGCTCAAAGGAGTCAAATTACATTACACAT |
| | AAACGAACTTATGGATTTGTTTATGAGAATCTTCACAATTGGAACTGTAACTTTGAAGCAAGGTGAA |
| | ATCAAGGATGCTACTCCTTCAGATTTTGTTCGCGCTACTGCAACGATACCGATACAAGCCTCACTCC |
| | CTTTCGGATGGCTTATTGTTGGCGTTGCACTTCTTGCTGTTTTTCAGAGCGCTTCCAAAATCATAAC |
| | CCTCAAAAAGAGATGGCAACTAGCACTCTCCAAGGGTGTTCACTTTGTTTGCAACTTGCTGTTGTTG |
| | TTTGTAACAGTTTACTCACACCTTTTGCTCGTTGCTGCTGGCCTTGAAGCCCCTTTTCTCTATCTTT |
| | ATGCTTTAGTCTACTTCTTGCAGAGTATAAACTTTGTAAGAATAATAATGAGGCTTTGGCTTTGCTG |
| | GAAATGCCGTTCCAAAAACCCATTACTTTATGATGCCAACTATTTTCTTTGCTGGCATACTAATTGT |
| | TACGACTATTGTATACCTTACAATAGTGTAACTTCTTCAATTGTCATTACTTCAGGTGATGGCACAA |
| | CAAGTCCTATTTCTGAACATGACTACCAGATTGGTGGTTATACTGAAAAATGGGAATCTGGAGTAAA |
| | AGACTGTGTTGTATTACACAGTTACTTCACTTCAGACTATTACCAGCTGTACTCAACTCAATTGAGT |
| | ACAGACACTGGTGTTGAACATGTTACCTTCTTCATCTACAATAAAATTGTTGATGAGCCTGAAGAAC |
| | ATGTCCAAATTCACACAATCGACGGTTCATCCGGAGTTGTTAATCCAGTAATGGAACCAATTTATGA |
| | TGAACCGACGACGACTACTAGCGTGCCTTTGTAAGCACAAGCTGATGAGTACGAACTTATGTACTCA |
| | TTCGTTTCGGAAGAGACAGGTACGTTAATAGTTAATAGCGTACTTCTTTTTCTTGCTTTCGTGGTAT |
| | TCTTGCTAGTTACACTAGCCATCCTTACTGCGCTTCGATTGTGTGCGTACTGCTGCAATATTGTTAA |
| | CGTGAGTCTTGTAAAACCTTCTTTTTACGTTTACTCTCGTGTTAAAAATCTGAATTCTTCTAGAGTT |
| | CCTGATCTTCTGGTCTAAACGAACTAAATATTATATTAGTTTTTCTGTTTGGAACTTTAATTTTAGC |
| | CATGGCAGATTCCAACGGTACTATTACCGTTGAAGAGCTTAAAAAGCTCCTTGAACAATGGAACCTA |
| | GTAATAGGTTTCCTATTCCTTACATGGATTTGTCTTCTACAATTTGCCTATGCCAACAGGAATAGGT |
| | TTTTGTATATAATTAAGTTAATTTTCCTCTGGCTGTTATGGCCAGTAACTTTAGCTTGTTTTGTGCT |
| | TGCTGCTGTTTACAGAATAAATTGGATCACCGGTGGAATTGCTATCGCAATGGCTTGTCTTGTAGGC |
| | TTGATGTGGCTCAGCTACTTCATTGCTTCTTTCAGACTGTTTGCGCGTACGCGTTCCATGTGGTCAT |
| | TCAATCCAGAAACTAACATTCTTCTCAACGTGCCACTCCATGGCACTATTCTGACCAGACCGCTTCT |
| | AGAAAGTGAACTCGTAATCGGAGCTGTGATCCTTCGTGGACATCTTCGTATTGCTGGACACCATCTA |
| | GGACGCTGTGACATCAAGGACCTGCCTAAAGAAATCACTGTTGCTACATCACGAACGCTTTCTTATT |
| | ACAAATTGGGAGCTTCGCAGCGTGTAGCAGGTGACTCAGGTTTTGCTGCATACAGTCGCTACAGGAT |
| | TGGCAACTATAAATTAAACACAGACCATTCCAGTAGCAGTGACAATATTGCTTTGCTTGTACAGTAA |
| | GTGACAACAGATGTTTCATCTCGTTGACTTTCAGGTTACTATAGCAGAGATATTACTAATTATTATG |
| | AGGACTTTTAAAGTTTCCATTTGGAATCTTGATTACATCATAAACCTCATAATTAAAAATTTATCTA |
| | AGTCACTAACTGAGAATAAATATTCTCAATTAGATGAAGAGCAACCAATGGAGATTGATTAAACGAA |
| | CATGAAAATTATTCTTTTCTTGGCACTGATAACACTCGCTACTTGTGAGCTTTATCACTACCAAGAG |
| | TGTGTTAGAGGTACAACAGTACTTTTAAAAGAACCTTGCTCTTCTGGAACATACGAGGGCAATTCAC |
| | CATNNNNNNNNNNNNNNNNNNNNNNNNNNNNNNNNNNNNNNNNNNNNNNNNNNNNNNNNNNNNNNNN |
| | NNNNNNNNGCGTAAAACACGTNNNNNNNNNNNNNNNNNNNNNNNNNNNNNNNNNNNNNNNNNNNNNN |
| | NNNNNNNNNNNNNNNNNNNNNNNNNNNNNNNNNNNNNNNNNNNNNNNNNNNNNNNNNNNNNNNNNNN |
| | NNNNNNNNNNNNNNNNNNNNNNNNNNNNNNNNNNNNNNNNNNNNNNNNNNNNNNNNNNNNGTGCTTT |
| | TTAGCCTTTCTGCTATTCCTTGTTTTAATTATGCTTATTATCTTTTGGTTCTCACTTGAACTGCAAG |
| | ATCATAATGAAACTTGTCACGCCTAAACGAACATGAAATTTCTTGTTTTCTTAGGAATCATCACAAC |
| | TGTAGCTGCATTTCACCAAGAATGTAGTTTACAGTCATGTACTTAACATCAACCATATGTAGTTGAT |
| | GACCCGTGTCCTATTCACTTCTATTCTAAATGGTATATTAGAGTAGGAGCTATAAAATCAGCACCTT |
| | TAATTGAATTGTGCGTGGATGAGGCTGGTTCTAAATCACCCATTCAGTGCATCGATATCGGTAATTA |
| | TACAGTTTCCTGTTTACCTTTTACAATTAATTGCCAGGAACCTAAATTGGGTAGTCTTGTAGTGCGT |
| | TGTTCGTTCTATGAAGACTTTTTAGAGTATCATGACGTTCGTGTTGTTTTAGATTTCATCTAAACGA |
| | ACAAACTAAATGTCTCTAAATGGACCCCAAAATCAGCGAAATGCACCCCGCATTACGTTTGGTGGAC |
| | CCTCAGATTCAACTGGCAGTAACCAGAATGGAGAACGCAGTGGGGCGCGATCAAAACAACGTCGGCC |
| | CCAAGGTTTACCCAATAATACTGCGTCTTGGTTCACCGCTCTCACTCAACATGGCAAGGAAGACCTT |
| | AAATTCCCTCGAGGACAAGGCGTTCCAATTAACACCAATAGCAGTCCAGATGACCAAATTGGCTACT |
| | ACCGAAGAGCTACCAGACGAATTCGTGGTGGTGACGGTAAAATGAAAGATCTCAGTCCAAGATGGTA |
| | TTTCTACTACCTAGGAACTGGGCCAGAAGCTGGACTTCCCTATGGTGCTAACAAAGACGGCATCATA |
| | TGGGTTGCAACTGAGGGAGCCTTGAATACACCAAAAGATCACATTGGCACCCGCAATCCTGCTAACA |
| | ATGCTGCAATCGTGCTACAACTTCCTCAAGGAACAACATTGCCAAAAGGCTTCTACGCAGAAGGGAG |
| | CAGAGGCGGCAGTCAAGCCTCTTCTCGTTCCTCATCACGTAGTCGCAACAGTTCAAGAAATTCAACT |
| | CCAGGCAGCAGTAAACGAACTTCTCCTGCTAGAATGGCTGGCAATGGCGGTGATGCTGCTCTTGCTT |
| | TGCTGCTGCTTGACAGATTGAACCAGCTTGAGAGCAAAATGTTTGGTAAAGGCCAACAACAACAAGG |
| | CCAAACTGTCACTAAGAAATCTGCTGCTGAGGCTTCTAAGAAGCCTCGGCAAAAACGTACTGCCACT |
| | AAAGCATACAATGTAACACAAGCTTTCGGCAGACGTGGTCCAGAACAAACCCAAGGAAATTTTGGGG |
| | ACCAGGAACTAATCAGACAAGGAACTGATTACAAACATTGGCCGCAAATTGCACAATTTGCCCCCAG |
| | CGCTTCAGCGTTCTTCGGAATGTCGCGCATTGGCATGGAAGTCACACCTTCGGGAACGTGGTTGACC |
| | TACACAGGTGCCATCAAATTGGATGACAAAGATCCAAATTTCAAAGATCAAGTCATTTTGCTGAATA |
| | AGCATATTGACGCATACAAAACATTCCCACCAACAGAGCCTAAAAAGGACAAAAAGAAGAAGGCTGA |
| | TGAAACTCAAGCCTTACCGCAGAGACAGAAGAAACAGCAAACTGTGACTCTTCTTCCTGCTGCAGAT |
| | TTGGATGATTTCTCCAAACAATTGCAACAATCCATGAGCAGTGCTGACTCAACTCAGGCCTAAACTC |
| | ATGCAGACCACACAAGGCAGATGGGCTATATAAACGTTTTCGCTTTTCCGTTTACGATATATAGTCT |
| | ACTCTTGTGCAGAATGAATTCTCGTAACTACATAGCACAAGTAGATGTAGTTAACTTTAATCTCACA |
| | TAGCAATCTTTAATCAGTGTGTAACATTAGGGAGGACTTGAAAGAGCCACCACATTTTCACCGAGGC |
| | CACGCGGAGTACGATCGAGTGTACAGTGAACAATGCTAGGGAGAGCTGCCTATATGGAAGAGCCCTA |
| | ATGTGTAAAATTAATTTTAGTAGTGCTATCCCCATGTGATTTTAATAGC |
| 291 | GenBank Accession No. MW692118.1(B.1.1.7 SARS-CoV-2 variant; Severe acute |
| | respiratory syndrome coronavirus 2 isolate SARS-CoV-2/human/LBN/Lebanon 7- |
| | B.1.1.7/2020) |
| | AGATCTGTTCTCTAAACGAACTTTAAAATCTGTGTGGCTGTCACTCGGCTGCATGCTTAGTGCACTC |
| | ACGCAGTATAATTAATAACTAATTACTGTCGTTGACAGGACACGAGTAACTCGTCTATCTTCTGCAG |
| | GCTGCTTACGGTTTCGTCCGTGTTGCAGCCGATCATCAGCACATCTAGGTTTTGTCCGGGTGTGACC |
| | GAAAGGTAAGATGGAGAGCCTTGTCCCTGGTTTCAACGAGAAAACACACGTCCAACTCAGTTTGCCT |
| | GTTTTACAGGTTCGCGACGTGCTCGTACGTGGCTTTGGAGACTCCGTGGAGGAGGTCTTATCAGAGG |
| | CACGTCAACATCTTAAAGATGGCACTTGTGGCTTAGTAGAAGTTGAAAAAGGCGTTTTGCCTCAACT |
| | TGAACAGCCCTATGTGTTCATCAAACGTTCGGATGCTCGAACTGCACCTCATGGTCATGTTATGGTT |
| | GAGCTGGTAGCAGAACTCGAAGGCATTCAGTACGGTCGTAGTGGTGAGACACTTGGTGTCCTTGTCC |
| | CTCATGTGGGCGAAATACCAGTGGCTTACCGCAAGGTTCTTCTTCGTAAGAACGGTAATAAAGGAGC |
| | TGGTGGCCATAGTTACGGCGCCGATCTAAAGTCATTTGACTTAGGCGACGAGCTTGGCACTGATCCT |
| | TATGAAGATTTTCAAGAAAACTGGAACACTAAACATAGCAGTGGTGTTACCCGTGAACTCATGCGTG |
| | AGCTTAACGGAGGGGCATACACTCGCTATGTCGATAACAACTTCTGTGGCCCTGATGGCTACCCTCT |
| | TGAGTGCATTAAAGACCTTCTAGCACGTGCTGGTAAAGCTTCATGCACTTTGTCTGAACAACTGGAC |
| | TTTATTGACACTAAGAGGGGTGTATACTGCTGCCGTGAACATGAGCATGAAATTGCTTGGTACACGG |
| | AACGTTCTGAAAAGAGCTATGAATTGCAGACACCTTTTGAAATTAAATTGGCAAAGAAATTTGACAC |
| | CTTCAATGGGGAATGTCCAAATTTTGTATTTCCCTTAAATTCCATAATCAAGACTATTCAACCAAGG |
| | GTTGAAAAGAAAAAGCTTGATGGCTTTATGGGTAGAATTCGATCTGTCTATCCAGTTGCGTCACCAA |
| | ATGAATGCAACCAAATGTGCCTTTCAACTCTCATGAAGTGTGATCATTGTGGTGAAACTTCATGGCA |
| | GACGGGCGATTTTGTTAAAGCCACTTGCGAATTTTGTGGCACTGAGAATTTGACTAAAGAAGGTGCC |
| | ACTACTTGTGGTTACTTACCCCAAAATGCTGTTGTTAAAATTTATTGTCCAGCATGTCACAATTCAG |
| | AAGTAGGACCTGAGCATAGTCTTGCCGAATACCATAATGAATCTGGCTTGAAAACCATTCTTCGTAA |
| | GGGTGGTCGCACTATTGCCTTTGGAGGCTGTGTGTTCTCTTATGTTGGTTGCCATAACAAGTGTGCC |
| | TATTGGGTTCCACGTGCTAGCGCTAACATAGGTTGTAACCATACAGGTGTTGTTGGAGAAGGTTCCG |
| | AAGGTCTTAATGACAACCTTCTTGAAATACTCCAAAAAGAGAAAGTCAACATCAATATTGTTGGTGA |
| | CTTTAAACTTAATGAAGAGATCGCCATTATTTTGGCATCTTTTTCTGCTTCCACAAGTGCTTTTGTG |
| | GAAACTGTGAAAGGTTTGGATTATAAAGCATTCAAACAAATTGTTGAATCCTGTGGTAATTTTAAAG |
| | TTACAAAAGGAAAAGCTAAAAAAGGTGCCTGGAATATTGGTGAACAGAAATCAATACTGAGTCCTCT |
| | TTATGCATTTGCATCAGAGGCTGCTCGTGTTGTACGATCAATTTTCTCCCGCACTCTTGAAACTGCT |
| | CAAAATTCTGTGCGTGTTTTACAGAAGGCCGCTATAACAATACTAGATGGAATTTCACAGTATTCAC |
| | TGAGACTCATTGATGCTATGATGTTCACATCTGATTTGGCTACTAACAATCTAGTTGTAATGGCCTA |
| | CATTACAGGTGGTGTTGTTCAGTTGACTTCGCAGTGGCTAACTAACATCTTTGGCACTGTTTATGAA |
| | AAACTCAAACCCGTCCTTGATTGGCTTGAAGAGAAGTTTAAGGAAGGTGTAGAGTTTCTTAGAGACG |
| | GTTGGGAAATTGTTAAATTTATCTCAACCTGTGCTTGTGAAATTGTCGGTGGACAAATTGTCACCTG |
| | TGCAAAGGAAATTAAGGAGAGTGTTCAGACATTCTTTAAGCTTGTAAATAAATTTTTGGCTTTGTGT |
| | GCTGACTCTATCATTATTGGTGGAGCTAAACTTAAAGCCTTGAATTTAGGTGAAACATTTGTCACGC |
| | ACTCAAAGGGATTGTACAGAAAGTGTGTTAAATCCAGAGAAGAAACTGGCCTACTCATGCCTCTAAA |
| | AGCCCCAAAAGAAATTATCTTCTTAGAGGGAGAAACACTTCCCACAGAAGTGTTAACAGAGGAAGTT |
| | GTCTTGAAAACTGGTGATTTACAACCATTAGAACAACCTACTAGTGAAGCTGTTGAAGCTCCATTGG |
| | TTGGTACACCAGTTTGTATTAACGGGCTTATGTTGCTCGAAATCAAAGACACAGAAAAGTACTGTGC |
| | CCTTGCACCTAATATGATGGTAACAAACAATACCTTCACACTCAAAGGCGGTGCACCAACAAAGGTT |
| | ACTTTTGGTGATGACACTGTGATAGAAGTGCAAGGTTACAAGAGTGTGAATATCACTTTTGAACTTG |
| | ATGAAAGGATTGATAAAGTACTTAATGAGAAGTGCTCTGCCTATACAGTTGAACTCGGTACAGAAGT |
| | AAATGAGTTCGCCTGTGTTGTGGCAGATGCTGTCATAAAAACTTTGCAACCAGTATCTGAATTACTT |
| | ACACCACTGGGCATTGATTTAGATGAGTGGAGTATGGCTACATACTACTTATTTGATGAGTCTGGTG |
| | AGTTTAAATTGGCTTCACATATGTATTGTTCTTTTTACCCTCCAGATGAGGATGAAGAAGAAGGTGA |
| | TTGTGAAGAAGAAGAGTTTGAGCCATCAACTCAATATGAGTATGGTACTGAAGATGATTAGCAAGGT |
| | AAACCTTTGGAATTTGGTGCCACTTCTGCTGCTCTTCAACCTGAAGAAGAGCAAGAAGAAGATTGGT |
| | TAGATGATGATAGTCAACAAACTGTTGGTCAACAAGACGGCAGTGAGGACAATCAGACAACTATTAT |
| | TCAAACAATTGTTGAGGTTCAACCTCAATTAGAGATGGAACTTACACCAGTTGTTCAGACTATTGAA |
| | GTGAATAGTTTTAGTGGTTATTTAAAACTTACTGACAATGTATACATTAAAAATGCAGACATTGTGG |
| | AAGAAGCTAAAAAGGTAAAACCAACAGTGGTTGTTAATGCAGCCAATGTTTACCTTAAACATGGAGG |
| | AGGTGTTGCAGGAGCCTTAAATAAGGCTACTAACAATGCCATGCAAGTTGAATCTGATGATTACATA |
| | GCTACTAATGGACCACTTAAAGTGGGTGGTAGTTGTGTTTTAAGCGGACACAATCTTGCTAAACACT |
| | GTCTTCATGTTGTCGGCCCAAATGTTAACAAAGGTGAAGACATTCAACTTCTTAAGAGTGCTTATGA |
| | AAATTTTAATCAGCACGAAGTTCTACTTGCACCATTATTATCAGCTGGTATTTTTGGTGCTGACCCT |
| | ATACATTCTTTAAGAGTTTGTGTAGATACTGTTCGCACAAATGTCTACTTAGCTGTCTTTGATAAAA |
| | ATCTCTATGACAAACTTGTTTCAAGCTTTTTGGAAATGAAGAGTGAAAAGCAAGTTGAACAAAAGAT |
| | CGCTGAGATTCCTAAAGAGGAAGTTAAGCCATTTATAACTGAAAGTAAACCTTCAGTTGAACAGAGA |
| | AAACAAGATGATAAGAAAATCAAAGCTTGTGTTGAAGAAGTTACAACAACTCTGGAAGAAACTAAGT |
| | TCCTCACAGAAAACTTGTTACTTTATATTGACATTAATGGCAATCTTCATCCAGATTCTGCCACTCT |
| | TGTTAGTGACATTGACATCACTTTCTTAAAGAAAGATGCTCCATATATAGTGGGTGATGTTGTTCAA |
| | GAGGGTGTTTTAACTGCTGTGGTTATACCTACTAAAAAGGCTGGTGGCACTACTGAAATGCTAGCGA |
| | AAGCTTTGAGAAAAGTGCCAACAGACAATTATATAACCACTTACCCGGGTCAGGGTTTAAATGGTTA |
| | CACTGTAGAGGAGGCAAAGACAGTGCTTAAAAAGTGTAAAAGTGCCTTTTACATTCTACCATCTATT |
| | ATCTCTAATGAGAAGCAAGAAATTCTTGGAACTGTTTCTTGGAATTTGCGAGAAATGCTTGCACATG |
| | CAGAAGAAACACGCAAATTAATGCCTGTCTGTGTGGAAACTAAAGCCATAGTTTCAACTATACAGCG |
| | TAAATATAAGGGTATTAAAATACAAGAGGGTGTGGTTGATTATGGTGCTAGATTTTACTTTTACACC |
| | AGTAAAACAACTGTAGCGTCACTTATCAACACACTTAACGATCTAAATGAAACTCTTGTTACAATGC |
| | CACTTGGCTATGTAACACATGGCTTAAATTTGGAAGAAGCTGCTCGGTATATGAGATCTCTCAAAGT |
| | GCCAGCTACAGTTTCTGTTTCTTCACCTGATGCTGTTACAGCGTATAATGGTTATCTTACTTCTTCT |
| | TCTAAAACACCTGAAGAACATTTTATTGAAACCATCTCACTTGCTGGTTCCTATAAAGATTGGTCCT |
| | ATTCTGGACAATCTACACAACTAGGTATAGAATTTCTTAAGAGAGGTGATAAAAGTGTATATTACAC |
| | TAGTAATCCTACCACATTCCACCTAGATGGTGAAGTTATCACCTTTGACAATCTTAAGACACTTCTT |
| | TCTTTGAGAGAAGTGAGGACTATTAAGGTGTTTACAACAGTAGACAACATTAACCTCCACACGCAAG |
| | TTGTGGACATGTCAATGACATATGGACAACAGTTTGGTCCAACTTATTTGGATGGAGCTGATGTTAC |
| | TAAAATAAAACCTCATAATTCACATGAAGGTAAAACATTTTATGTTTTACCTAATGATGACACTCTA |
| | CGTGTTGAGGCTTTTGAGTACTACCACACAACTGATCCTAGTTTTCTGGGTAGGTACATGTCAGCAT |
| | TAAATCACACTAAAAAGTGGAAATACCCACAAGTTAATGGTTTAACTTCTATTAAATGGGCAGATAA |
| | CAACTGTTATCTTGCCACTGCATTGTTAACACTCCAACAAATAGAGTTGAAGTTTAATCCACCTGCT |
| | CTACAAGATGCTTATTACAGAGCAAGGGCTGGTGAAGCTGATAACTTTTGTGCACTTATCTTAGCCT |
| | ACTGTAATAAGACAGTAGGTGAGTTAGGTGATGTTAGAGAAACAATGAGTTACTTGTTTCAACATGC |
| | CAATTTAGATTCTTGCAAAAGAGTCTTGAACGTGGTGTGTAAAACTTGTGGACAACAGCAGACAACC |
| | CTTAAGGGTGTAGAAGCTGTTATGTACATGGGCACACTTTCTTATGAACAATTTAAGAAAGGTGTTC |
| | AGATACCTTGTACGTGTGGTAAACAAGCTACAAAATATCTAGTACAACAGGAGTCACCTTTTGTTAT |
| | GATGTCAGCACCACCTGCTCAGTATGAACTTAAGCATGGTACATTTACTTGTGCTAGTGAGTACACT |
| | GGTAATTACCAGTGTGGTCACTATAAACATATAACTTCTAAAGAAACTTTGTATTGCATAGACGGTG |
| | CTTTACTTACAAAGTCCTCAGAATACAAAGGTCCTATTACGGATGTTTTCTACAAAGAAAACAGTTA |
| | CACAACAACCATAAAACCAGTTACTTATAAATTGGATGGTGTTGTTTGTACAGAAATTGACCCTAAG |
| | TTGGACAATTATTATAAGAAAGACAATTCTTATTTTACAGAGCAACCAATTGATCTTGTACCAAACC |
| | AACCATATCCAAACGCAAGCTTCGATAATTTTAAGTTTGTATGTGATAATATCAAATTTGCTGATGA |
| | TTTAAACCAGTTAACTGGTTATAAGAAACCTGCTTCAAGAGAGCTTAAAGTTACATTTTTCCCTGAC |
| | TTAAATGGTGATGTGGTGGCTATTGATTATAAACACTACACACCCTCTTTTAAGAAAGGAGCTAAAT |
| | TGTTACATAAACCTATTGTTTGGCATGTTAACAATGCAACTAATAAAGCCACGTATAAACCAAATAC |
| | CTGGTGTATACGTTGTCTTTGGAGCACAAAACCAGTTGAAACATCAAATTCGTTTGATGTACTGAAG |
| | TCAGAGGACGCGCAGGGAATGGATAATCTTGCCTGCGAAGATCTAAAACCAGTCTCTGAAGAAGTAG |
| | TGGAAAATCCTACCATACAGAAAGACGTTCTTGAGTGTAATGTGAAAACTACCGAAGTTGTAGGAGA |
| | CATTATACTTAAACCAGCAAATAATAGTTTAAAAATTACAGAAGAGGTTGGCCACACAGATCTAATG |
| | GCTGCTTATGTAGACAATTCTAGTCTTACTATTAAGAAACCTAATGAATTATCTAGAGTATTAGGTT |
| | TGAAAACCCTTGCTACTCATGGTTTAGCTGCTGTTAATAGTGTCCCTTGGGATACTATAGCTAATTA |
| | TGCTAAGCCTTTTCTTAACAAAGTTGTTAGTACAACTACTAACATAGTTACACGGTGTTTAAACCGT |
| | GTTTGTACTAATTATATGCCTTATTTCTTTACTTTATTGCTACAATTGTGTACTTTTACTAGAAGTA |
| | CAAATTCTAGAATTAAAGCATCTATGCCGACTACTATAGCAAAGAATACTGTTAAGAGTGTCGGTAA |
| | ATTTTGTCTAGAGGCTTCATTTAATTATTTGAAGTCACCTAATTTTTCTAAACTGATAAATATTACA |
| | ATTTGGTTTTTACTATTAAGTGTTTGCCTAGGTTCTTTAATCTACTCAACCGCTGCTTTAGGTGTTT |
| | TAATGTCTAATTTAGGCATGCCTTCTTACTGTACTGGTTACAGAGAAGGCTATTTGAACTCTACTAA |
| | TGTCACTATTGCAACCTACTGTACTGGTTCTATACCTTGTAGTGTTTGTCTTAGTGGTTTAGATTCT |
| | TTAGACACCTATCCTTCTTTAGAAACTATACAAATTACCATTTCATCTTTTAAATGGGATTTAACTG |
| | CTTTTGGCTTAGTTGCAGAGTGGTTTTTGGCATATATTCTTTTCACTAGGTTTTTCTATGTACTTGG |
| | ATTGGCTGCAATCATGCAATTGTTTTTCAGCTATTTTGCAGTACATTTTATTAGTAATTCTTGGCTT |
| | ATGTGGTTAATAATTAATCTTGTACAAATGGCCCCGATTTCAGCTATGGTTAGAATGTACATCTTCT |
| | TTGCATCATTTTATTATGTATGGAAAAGTTATGTGCATGTTGTAGACGGTTGTAATTCATCAACTTG |
| | TATGATGTGTTACAAACGTAATAGAGCAACAAGAGTCGAATGTACAACTATTGTTAATGGTGTTAGA |
| | AGGTCCTTTTATGTCTATGCTAATGGAGGTAAAGGCTTTTGCAAACTACACAATTGGAATTGTGTTA |
| | ATTGTGATACATTCTGTGCTGGTAGTACATTTATTAGTGATGAAGTNNNNNNNNNNNNNNNNNNNNN |
| | NNNNNNNNNNNNNNNNNNNNNNNNNNNNNNNNNNNNNNNNNNNNNNNNNNNNNNNNNNNNNNNNNNN |
| | NNNNNNNNNNNNNNNNNNNNNNNNNNNNNNNNNNNNNNNNNNNNNNNNNNNNNNNNNNNNNNNNNNN |
| | NNNNNNNNNNNNNNNNNNNNNNNNNNNNNNNNNNNNNNNNNNNNNNNNNNNNNNNNNNNNNNNNNNN |
| | NNNNNNNNNNNNNNNNNNNNNNNNNNNNNNNNNNNNNNNNNNNNNNNNNNNNNNNNNNNNNNNNNNN |
| | NNNNNNNNTATACTGTTACTAGATCAGGCATTAGTGTCTGATGTTGGTGATAGTGCGGAAGTTGCAG |
| | TTAAAATGTTTGATGCTTACGTTAATACGTTTTCATCAACTTTTAACGTACCAATGGAAAAACTCAA |
| | AACACTAGTTGCAACTGCAGAAGCTGAACTTGCAAAGAATGTGTCCTTAGACAATGTCTTATCTACT |
| | TTTATTTCAGCAGCTCGGCAAGGGTTTGTTGATTCAGATGTAGAAACTAAAGATGTTGTTGAATGTC |
| | TTAAATTGTCACATCAATCTGACATAGAAGTTACTGGCGATAGTTGTAATAACTATATGCTCACCTA |
| | TAACAAAGTTGAAAACATGACACCCCGTGACCTTGGTGCTTGTATTGACTGTAGTGCGCGTCATATT |
| | AATGCGCAGGTAGCAAAAAGTCACAACATTGCTTTGATATGGAACGTTAAAGATTTCATGTCATTGT |
| | CTGAACAACTACGAAAACAAATACGTAGTGCTGCTAAAAAGAATAACTTACCTTTTAAGTTGACATG |
| | TGCAACTACTAGACAAGTTGTTAATGTTGTAACAACAAAGATAGCACTTAAGGGTGGTAAAATTGTT |
| | AATAATTGGTTGAAGCAGTTAATTAAAGTTACACTTGTGTTCCTTTTTGTTGCTGCTATTTTCTATT |
| | TAATAACACCTGTTCATGTCATGTCTAAACATACTGACTTTTCAAGTGAAATCATAGGATACAAGGC |
| | TATTGATGGTGGTGTCACTCGTGACATAGCATCTACAGATACTTGTTTTGCTAACAAACATGCTGAT |
| | TTTGACACATGGTTTAGCCAGCGTGGTGGTAGTTATACTAATGACAAAGCTTGCCCATTGATTGCTG |
| | CAGTCATAACAAGAGAAGTGGGTTTTGTCGTGCCTGGTTTGCCTGGCACGATATTACGCACAACTAA |
| | TGGTGACTTTTTGCATTTCTTACCTAGAGTTTTTAGTGCAGTTGGTAACATCTGTTACACACCATCA |
| | AAACTTATAGAGTACACTGACTTTGCAACATCAGCTTGTGTTTTGGCTGCTGAATGTACAATTTTTA |
| | AAGATGCTTCTGGTAAGCCAGTACCATATTGTTATGATACCAATGTACTAGAAGGTTCTGTTGCTTA |
| | TGAAAGTTTACGCCCTGACACACGTTATGTGCTCATGGATGGCTCTATTATTCAATTTCCTAACACC |
| | TACCTTGAAGGTTCTGTTAGAGTGGTAACAACTTTTGATTCTGAGTACTGTAGGCACGGCACTTGTG |
| | AAAGATCAGAAGCTGGTGTTTGTGTATCTACTAGTGGTAGATGGGTACTTAACAATGATTATTACAG |
| | ATCTTTACCAGGAGTTTTCTGTGGTGTAGATGCTGTAAATTTACTTACTAATATGTTTACACCACTA |
| | ATTCAACCTATTGGTGCTTTGGACATATCAGCATCTATAGTAGCTGGTGGTATTGTAGCTATCGTAG |
| | TAACATGCCTTGCCTACTATTTTATGAGGTTTAGAAGAGCTTTTGGTGAATACAGTCATGTAGTTGC |
| | CTTTAATACTTTACTATTCCTTATGTCATTCACTGTACTCTGTTTAACACCAGTTTACTCATTCTTA |
| | CCTGGTGTTTATTCTGTTATTTACTTGTACTTGACATTTTATCTTACTAATGATGTTTCTTTTTTAG |
| | CACATATTCAGTGGATGGTTATGTTCACACCTTTAGTACCTTTCTGGATAACAATTGCTTATATCAT |
| | TTGTATTTCCACAAAGCATTTCTATTGGTTCTTTAGTAATTACCTAAAGAGACGTGTAGTCTTTAAT |
| | GGTGTTTCCTTTAGTACTTTTGAAGAAGCTGCGCTGTGCACCTTTTTGTTAAATAAAGAAATGTATC |
| | TAAAGTTGCGTAGTGATGTGCTATTACCTCTTACGCAATATAATAGATACTTAGCTCTTTATAATAA |
| | GTACAAGTATTTTAGTGGAGCAATGGATACAACTAGCTACAGAGAAGCTGCTTGTTGTCATCTCGCA |
| | AAGGCTCTCAATGACTTCAGTAACTCAGGTTCTGATGTTCTTTACCAACCACCACAAACCTCTATCA |
| | CCTCAGCTGTTTTGCAGAGTGGTTTTAGAAAAATGGCATTCCCATCTGGTAAAGTTGAGGGTTGTAT |
| | GGTACAAGTAACTTGTGGTACAACTACACTTAACGGTCTTTGGCTTGATGACGTAGTTTACTGTCCA |
| | AGACATGTGATCTGCACCTCTGAAGACATGCTTAACCCTAATTATGAAGATTTACTCATTCGTAAGT |
| | CTAATCATAATTTCTTGGTACAGGCTGGTAATGTTCAACTCAGGGTTATTGGACATTCTATGCAAAA |
| | TTGTGTACTTAAGCTTAAGGTTGATACAGCCAATCCTAAGACACCTAAGTATAAGTTTGTTCGCATT |
| | CAACCAGGACAGACTTTTTCAGTGTTAGCTTGTTACAATGGTTCACCATCTGGTGTTTACCAATGTG |
| | CTATGAGGCCCAATTTCACTATTAAGGGTTCATTCCTTAATGGTTCATGTGGTAGTGTTGGTTTTAA |
| | CATAGATTATGACTGTGTCTCTTTTTGTTACATGCACCATATGGAATTACCAACTGGAGTTCATGCT |
| | GGCACAGACTTAGAAGGTAACTTTTATGGACCTTTTGTTGACAGGCAAACAGCACAAGCAGCTGGTA |
| | CGGACACAACTATTACAGTTAATGTTTTAGCTTGGTTGTACGCTGCTGTTATAAATGGAGACAGGTG |
| | GTTTCTCAATCGATTTACCACAACTCTTAATGACTTTAACCTTGTGGCTATGAAGTACAATTATGAA |
| | CCTCTAACACAAGACCATGTTGACATACTAGGACCTCTTTCTGCTCAAACTGGAATTGCCGTTTTAG |
| | ATATGTGTGCTTCATTAAAAGAATTACTGCAAAATGGTATGAATGGACGTACCATATTGGGTAGTGC |
| | TTTATTAGAAGATGAATTTACACCTTTTGATGTTGTTAGACAATGCTCAGGTGTTACTTTCCAAAGT |
| | GCAGTGAAAAGAACAATCAAGGGTACACACCACTGGTTGTTACTCACAATTTTGACTTCACTTTTAG |
| | TTTTAGTCCAGAGTACTCAATGGTCTTTGTTCTTTTTTTTGTATGAAAATGCCTTTTTACCTTTTGC |
| | TATGGGTATTATTGCTATGTCTGCTTTTGCAATGATGTTTGTCAAACATAAGCATGCATTTCTCTGT |
| | TTGTTTTTGTTACCTTCTCTTGCCACTGTAGCTTATTTTAATATGGTCTATATGCCTGCTAGTTGGG |
| | TGATGCGTATTATGACATGGTTGGATATGGTTGATACTAGTTTGAAGCTAAAAGACTGTGTTATGTA |
| | TGCATCAGCTGTAGTGTTACTAATCCTTATGACAGCAAGAACTGTGTATGATGATGGTGCTAGGAGA |
| | GTGTGGACACTTATGAATGTCTTGACACTCGTTTATAAAGTTTATTATGGTAATGCTTTAGATCAAG |
| | CCATTTCCATGTGGGCTCTTATAATCTCTGTTACTTCTAACTACTCAGGTGTAGTTACAACTGTCAT |
| | GTTTTTGGCCAGAGGTATTGTTTTTATGTGTGTTGAGTATTGCCCTATTTTCTTCATAACTGGTAAT |
| | ACACTTCAGTGTATAATGCTAGTTTATTGTTTCTTAGGCTATTTTTGTACTTGTTACTTTGGCCTCT |
| | TTTGTTTACTCAACCGCTACTTTAGACTGACTCTTGGTGTTTATGATTACTTAGTTTCTACACAGGA |
| | GTTTAGATATATGAATTCACAGGGACTACTCCCACCCAAGAATAGCATAGATGCCTTCAAACTCAAC |
| | ATTAAATTGTTGGGTGTTGGTGGCAAACCTTGTATCAAAGTAGCCACTGTACAGTCTAAAATGTCAG |
| | ATGTAAAGTGCACATCAGTAGTCTTACTCTCAGTTTTGCAACAACTCAGAGTAGAATCATCATCTAA |
| | ATTGTGGGCTCAATGTGTCCAGTTACACAATGACATTCTCTTAGCTAAAGATACTACTGAAGCCTTT |
| | GAAAAAATGGTTTCACTACTTTCTGTTTTGCTTTCCATGCAGGGTGCTGTAGACATAAACAAGCTTT |
| | GTGAAGAAATGCTGGACAACAGGGCAACCTTACAAGCTATAGCCTCAGAGTTTAGTTCCCTTCCATC |
| | ATATGCAGCTTTTGCTACTGCTCAAGAAGCTTATGAGCAGGCTGTTGCTAATGGTGATTCTGAAGTT |
| | GTTCTTAAAAAGTTGAAGAAGTCTTTGAATGTGGCTAAATCTGAATTTGACCGTGATGCAGCCATGC |
| | AACGTAAGTTGGAAAAGATGGCTGATCAAGCTATGACCCAAATGTATAAACAGGCTAGATCTGAGGA |
| | CAAGAGGGCAAAAGTTACTAGTGCTATGCAGACAATGCTTTTCACTATGCTTAGAAAGTTGGATAAT |
| | GATGCACTCAACAACATTATCAACAATGCAAGAGATGGTTGTGTTCCCTTGAACATAATACCTCTTA |
| | CAACAGCAGCCAAACTAATGGTTGTCATACCAGACTATAACACATATAAAAATACGTGTGATGGTAC |
| | AACATTTACTTATGCATCAGCATTGTGGGAAATCCAACAGGTTGTAGATGCAGATAGTAAAATTGTT |
| | CAACTTAGTGAAATTAGTATGGACAATTCACCTAATTTAGCATGGCCTCTTATTGTAACAGCTTTAA |
| | GGGCCAATTCTGCTGTCAAATTACAGAATAATGAGCTTAGTCCTGTTGCACTACGACAGATGTCTTG |
| | TGCTGCCGGTACTACACAAACTGCTTGCACTGATGACAATGCGTTAGCTTACTACAACACAACAAAG |
| | GGAGGTAGGTTTGTACTTGCACTGTTATCCGATTTACAGGATTTGAAATGGGCTAGATTCCCTAAGA |
| | GTGATGGAACTGGTACTATCTATACAGAACTGGAACCACCTTGTAGGTTTGTTACAGACACACCTAA |
| | AGGTCCTAAAGTGAAGTATTTATACTTTATTAAAGGATTAAACAACCTAAATAGAGGTATGGTACTT |
| | GGTAGTTTAGCTGCCACAGTACGTCTACAAGCTGGTAATGCAACAGAAGTGCCTGCCAATTCAACTG |
| | TATTATCTTTCTGTGCTTTTGCTGTAGATGCTGCTAAAGCTTACAAAGATTATCTAGCTAGTGGGGG |
| | ACAACCAATCACTAATTGTGTTAAGATGTTGTGTACACACACTGGTACTGGTCAGGCAATAACAGTT |
| | ACACCGGAAGCCAATATGGATCAAGAATCCTTTGGTGGTGCATCGTGTTGTCTGTACTGCCGTTGCC |
| | ACATAGATCATCCAAATCCTAAAGGATTTTGTGACTTAAAAGGTAAGTATGTACAAATACCTACAAC |
| | TTGTGCTAATGACCCTGTGGGTTTTACACTTAAAAACACAGTCTGTACCGTCTGCGGTATGTGGAAA |
| | GGTTATGGCTGTAGTTGTGATCAACTCCGCGAACCCATGCTTCAGTCAGCTGATGCACAATCGTTTT |
| | TAAACGGGTTTGCGGTGTAAGTGCAGCCCGTCTTACACCGTGCGGCACAGGCACTAGTACTGATGTC |
| | GTATACAGGGCTTTTGACATCTACAATGATAAAGTAGCTGGTTTTGCTAAATTCCTAAAAACTAATT |
| | GTTGTCGCTTCCAAGAAAAGGACGAAGATGACAATTTAATTGATTCTTACTTTGTAGTTAAGAGACA |
| | CACTTTCTCTAACTACCAACATGAAGAAACAATTTATAATTTACTTAAGGATTGTCCAGCTGTTGCT |
| | AAACATGACTTCTTTAAGTTTAGAATAGACGGTGACATGGTACCACATATATCACGTCAACGTCTTA |
| | CTAAATACACAATGGCAGACCTCGTCTATGCTTTAAGGCATTTTGATGAAGGTAATTGTGACACATT |
| | AAAAGAAATACTTGTCACATACAATTGTTGTGATGATGATTATTTCAATAAAAAGGACTGGTATGAT |
| | TTTGTAGAAAACCCAGATATATTACGCGTATACGCCAACTTAGGTGAACGTGTACGCCAAGCTTTGT |
| | TAAAAACAGTACAATTCTGTGATGCCATGCGAAATGCTGGTATTGTTGGTGTACTGACATTAGATAA |
| | TCAAGATCTCAATGGTAACTGGTATGATTTCGGTGATTTCATACAAACCACGCCAGGTAGTGGAGTT |
| | CCTGTTGTAGATTCTTATTATTCATTGTTAATGCCTATATTAACCTTGACCAGGGCTTTAACTGCAG |
| | AGTCACATGTTGACACTGACTTAACAAAGCCTTACATTAAGTGGGATTTGTTAAAATATGACTTCAC |
| | GGAAGAGAGGTTAAAACTCTTTGACCGTTATTTTAAATATTGGGATCAGACATACCACCCAAATTGT |
| | GTTAACTGTTTGGATGACAGATGCATTCTGCATTGTGCAAACTTTAATGTTTTATTCTCTACAGTGT |
| | TCCCACTTACAAGTTTTGGACCACTAGTGAGAAAAATATTTGTTGATGGTGTTCCATTTGTAGTTTC |
| | AACTGGATACCACTTCAGAGAGCTAGGTGTTGTACATAATCAGGATGTAAACTTACATAGCTCTAGA |
| | CTTAGTTTTAAGGAATTACTTGTGTATGCTGCTGACCCTGCTATGCACGCTGCTTCTGGTAATCTAT |
| | TACTAGATAAACGCACTACGTGCTTTTCAGTAGCTGCACTTACTAACAATGTTGCTTTTCAAACTGT |
| | CAAACCTGGTAATTTTAACAAAGACTTCTATGACTTTGCTGTGTCTAAGGGTTTCTTTAAGGAAGGA |
| | AGTTCTGTTGAATTAAAACACTTCTTCTTTGCTCAGGATGGTAATGCTGCTATCAGCGATTATGACT |
| | ACTATCGTTATAATCTACCAACAATGTGTGATATCAGACAACTACTATTTGTAGTTGAAGTTGTTGA |
| | TAAGTACTTTGATTGTTACGATGGTGGCTGTATTAATGCTAACCAAGTCATCGTCAACAACCTAGAC |
| | AAATCAGCTGGTTTTCCATTTAATAAATGGGGTAAGGCTAGACTTTATTATGATTCAATGAGTTATG |
| | AGGATCAAGATGCACTTTTCGCATATACAAAACGTAATGTCATCCCTACTATAACTCAAATGAATCT |
| | TAAGTATGCCATTAGTGCAAAGAACAGAGCTCGCACCGTAGCTGGTGTCTCTATCTGTAGTACTATG |
| | ACCAATAGACAGTTTCATCAAAAATTATTGAAATCAATAGCCGCCACTAGAGGAGCTACTGTAGTAA |
| | TTGGAACAAGCAAATTCTATGGTGGTTGGCACAACATGTTAAAAACTGTTTATAGTGATGTAGAAAA |
| | CCCTCATCTTATGGGTTGGGATTATCCTAAATGTGATAGAGCCATGCCTAACATGCTTAGAATTATG |
| | GCCTCACTTGTTCTTGCTCGCAAACATACAACGTGTTGTAGCTTGTCACACCGTTTCTATAGATTAG |
| | CTAATGAGTGTGCTCAAGTATTGAGTGAAATGGTCATGTGTGGCGGTTCACTATATGTTAAACCAGG |
| | TGGAACCTCATCAGGAGATGCCACAACTGCTTATGCTAATAGTGTTTTTAACATTTGTCAAGCTGTC |
| | ACGGCCAATGTTAATGCACTTTTATCTACTGATGGTAACAAAATTGCCGATAAGTATGTCCGCAATT |
| | TACAACACAGACTTTATGAGTGTCTCTATAGAAATAGAGATGTTGACACAGACTTTGTGAATGAGTT |
| | TTACGCATATTTGCGTAAACATTTCTCAATGATGATACTCTCTGACGATGCTGTTGTGTGTTTCAAT |
| | AGCACTTATGCATCTCAAGGTCTAGTGGCTAGCATAAAGAACTTTAAGTCAGTTCTTTATTATCAAA |
| | ACAATGTTTTTATGTCTGAAGCAAAATGTTGGACTGAGACTGACCTTACTAAAGGACCTCATGAATT |
| | TTGCTCTCAACATACAATGCTAGTTAAACAGGGTGATGATTATGTGTACCTTCCTTACCCAGATCCA |
| | TCAAGAATCCTAGGGGCCGGCTGTTTTGTAGATGATATCGTAAAAACAGATGGTACACTTATGATTG |
| | AACGGTTCGTGTCTTTAGCTATAGATGCTTACCCACTTACTAAACATCCTAATCAGGAGTATGCTGA |
| | TGTCTTTCATTTGTACTTACAATACATAAGAAAGCTACATGATGAGTTAACAGGACACATGTTAGAC |
| | ATGTATTCTGTTATGCTTACTAATGATAACACCTCAAGGTATTGGGAACCTGAGTTTTATGAGGCTA |
| | TGTACACACCGCATACAGTCTTACAGGCTGTTGGGGCTTGTGTTCTTTGCAATTCACAGACTTCATT |
| | AAGATGTGGTGCTTGCATACGTAGACCATTCTTATGTTGTAAATGCTGTTACGACCATGTCATATCA |
| | ACATCACATAAATTAGTCTTGTCTGTTAATCCGTATGTTTGCAATGCTCCAGGTTGTGATGTCACAG |
| | ATGTGACTCAACTTTACTTAGGAGGTATGAGCTATTATTGTAAATCACATAAACCACCCATTAGTTT |
| | TCCATTGTGTGCTAATGGACAAGTTTTTGGTTTATATAAAAATACATGTGTTGGTAGCGATAATGTT |
| | ACTGACTTTAATGCAATTGCAACATGTGACTGGACAAATGCTGGTGATTACATTTTAGCTAACACCT |
| | GTACTGAAAGACTCAAGCTTTTTGCAGCAGAAACGCTCAAAGCTACTGAGGAGACATTTAAACTGTC |
| | TTATGGTATTGCTACTGTACGTGAAGTGCTGTCTGACAGAGAATTACATCTTTCATGGGAAGTTGGT |
| | AAACCTAGACCACCACTTAACCGAAATTATGTCTTTACTGGTTATCGTGTAACTAAAAACAGTAAAG |
| | TACAAATAGGAGAGTACACCTTTGAAAAAGGTGACTATGGTGATGCTGTTGTTTACCGAGGTACAAC |
| | AACTTACAAATTAAATGTTGGTGATTATTTTGTGCTGACATCACATACAGTAATGCCATTAAGTGCA |
| | CCTACACTAGTGCCACAAGAGCACTATGTTAGAATTACTGGCTTATACCCAACACTCAATATCTCAG |
| | ATGAGTTTTCTAGCAATGTTGCAAATTATCAAAAGGTTGGTATGCAAAAGTATTCTACACTCCAGGG |
| | ACCACCTGGTACTGGTAAGAGTCATTTTGCTATTGGCCTAGCTCTCTACTACCCTTCTGCTCGCATA |
| | GTGTATACAGCTTGCTCTCATGCCGCTGTTGATGCACTATGTGAGAAGGCATTAAAATATTTGCCTA |
| | TAGATAAATGTAGTAGAATTATACCTGCACGTGCTCGTGTAGAGTGTTTTGATAAATTCAAAGTGAA |
| | TTCAACATTAGAACAGTATGTCTTTTGTACTGTAAATGCATTGCCTGAGACGACAGCAGATATAGTT |
| | GTCTTTGATGAAATTTCAATGGCCACAAATTATGATTTGAGTGTTGTCAATGCCAGATTACGTGCTA |
| | AGCACTATGTGTACATTGGCGACCCTGCTCAATTACCTGCACCACGCACATTGCTAACTAAGGGCAC |
| | ACTAGAACCAGAATATTTCAATTCAGTGTGTAGACTTATGAAAACTATAGGTCCAGACATGTTCCTC |
| | GGAACTTGTCGGCGTTGTCCTGCTGAAATTGTTGACACTGTGAGTGCTTTGGTTTATGATAATAAGC |
| | TTAAAGCACATAAAGACAAATCAGCTCAATGCTTTAAAATGTTTTATAAGGGTGTTATCACGCATGA |
| | TGTTTCATCTGCAATTAACAGGCCACAAATAGGCGTGGTAAGAGAATTCCTTACACGTAACCCTGCT |
| | TGGAGAAAAGCTGTCTTTATTTCACCTTATAATTCACAGAATGCTGTAGCCTCAAAGATTTTGGGAC |
| | TACCAACTCAAACTGTTGATTCATCACAGGGCTCAGAATATGACTATGTCATATTCACTCAAACCAC |
| | TGAAACAGCTCACTCTTGTAATGTAAACAGATTTAATGTTGCTATTACCAGAGCAAAAGTAGGCATA |
| | CTTTGCATAATGTCTGATAGAGACCTTTATGACAAGTTGCAATTTACAAGTCTTGAAATTCCACGTA |
| | GGAATGTGGCAACTTTACAAGCTGAAAATGTAACAGGACTCTTTAAAGATTGTAGTAAGGTAATCAC |
| | TGGGTTACATCCTACACAGGCACCTACACACCTCAGTGTTGACACTAAATTCAAAACTGAAGGTTTA |
| | TGTGTTGACATACCTGGCATACCTAAGGACATGACCTATAGAAGACTCATCTCTATGATGGGTTTTA |
| | AAATGAATTATCAAGTTAATGGTTACCCTAACATGTTTATCACCCGCGAAGAAGCTATAAGACATGT |
| | ACGTGCATGGATTGGCTTCGATGTCGAGGGGTGTCATGCTACTAGAGAAGCTGTTGGTACCAATTTA |
| | CCTTTACAGCTAGGTTTTTCTACAGGTGTTAACCTAGTTGCTGTACCTACAGGTTATGTTGATACAC |
| | CTAATAATACAGATTTTTCCAGAGTTAGTGCTAAACCACCGCCTGGAGATCAATTTAAACACCTCAT |
| | ACCACTTATGTACAAAGGACTTCCTTGGAATGTAGTGCGTATAAAGATTGTACAAATGTTAAGTGAC |
| | ACACTTAAAAATCTCTCTGACAGAGTCGTATTTGTCTTATGGGCACATGGCTTTGAGTTGACATCTA |
| | TGAAGTATTTTGTGAAAATAGGACCTGAGCGCACCTGTTGTCTATGTGATAGACGTGCCACATGCTT |
| | TTCCACTGCTTCAGACACTTATGCCTGTTGGCATCATTCTATTGGATTTGATTACGTCTATAATCCG |
| | TTTATGATTGATGTTCAACAATGGGGTTTTACAGGTAACCTACAAAGCAACCATGATCTGTATTGTC |
| | AAGTCCATGGTAATGCACATGTAGCTAGTTGTGATGCAATCATGACTAGGTGTCTAGCTGTCCACGA |
| | GTGCTTTGTTAAGCGTGTTGACTGGACTATTGAATATCCTATAATTGGTGATGAACTGAAGATTAAT |
| | GCGGCTTGTAGAAAGGTTCAACACATGGTTGTTAAAGCTGCATTATTAGCAGACAAATTCCCAGTTC |
| | TTCACGACATTGGTAACCCTAAAGCTATTAAGTGTGTACCTCAAGCTGATGTAGAATGGAAGTTCTA |
| | TGATGCACAGCCTTGTAGTGACAAAGCTTATAAAATAGAAGAATTATTCTATTCTTATGCCACACAT |
| | TCTGACAAATTCACAGATGGTGTATGCCTATTTTGGAATTGCAATGTCGATAGATATCCTGCTAATT |
| | CCATTGTTTGTAGATTTGACACTAGAGTGCTATCTAACCTTAACTTGCCTNNNNNNNNNNNNNNNNN |
| | NNNNNNNNNNNNNNNNNNNNNNNNNNNNNNNNNNNNNNNNNNNNNNNNNNNNNNNNNNNNNNNNNNN |
| | NNNNNNNNNNNNNNNNNNNNNNNNNNNNNNNNNNNNNNNNNNNNNNNNNNNNNNNNNNNNNNNNNNN |
| | NNNNNNNNNNNNNNNNNNNNNNNNNNNNNNNNNNNNNNNNNNNNNNNNNNNNNNNNNNNNNNNNNNN |
| | NNNNNNNNNNNNNNNNNNNNNNNNNNNNNNNNNNNNNNNNNNNNNNNNNNNNNNNNNNNNNNNNNNN |
| | NNNNNNNNNNACAAACAATTTGATACTTATAACCTCTGGAACACTTTTACAAGACTTCAGAGTTTAG |
| | AAAATGTGGCTTTTAATGTTGTAAATAAGGGACACTTTGATGGACAACAGGGTGAAGTACCAGTTTC |
| | TATCATTAATAACACTGTTTACACAAAAGTTGATGGTGTTGATGTAGAATTGTTTGAAAATAAAACA |
| | ACATTACCTGTTAATGTAGCATTTGAGCTTTGGGCTAAGCGCAACATTAAACCAGTACCAGAGGTGA |
| | AAATACTCAATAATTTGGGTGTGGACATTGCTGCTAATACTGTGATCTGGGACTACAAAAGAGATGC |
| | TCCAGCACATATATCTACTATTGGTGTTTGTTCTATGACTGACATAGCCAAGAAACCAACTGAAACG |
| | ATTTGTGCACCACTCACTGTCTTTTTTGATGGTAGAGTTGATGGTCAAGTAGACTTATTTAGAAATG |
| | CCCGTAATGGTGTTCTTATTACAGAAGGTAGTGTTAAAGGTTTACAACCATCTGTAGGTCCCAAACA |
| | AGCTAGTCTTAATGGAGTCACATTAATTGGAGAAGCCGTAAAAACACAGTTCAATTATTATAAGAAA |
| | GTTGATGGTGTTGTCCAACAATTACCTGAAACTTACTTTACTCAGAGTAGAAATTTACAAGAATTTA |
| | AACCCAGGAGTCAAATGGAAATTGATTTCTTAGAATTAGCTATGGATGAATTCATTGAACGGTATAA |
| | ATTAGAAGGCTATGCCTTCGAACATATCGTTTATGGAGATTTTAGTCATAGTCAGTTAGGTGGTTTA |
| | CATCTACTGATTGGACTAGCTAAACGTTTTAAGGAATCACCTTTTGAATTAGAAGATTTTATTCCTA |
| | TGGACAGTACAGTTAAAAACTATTTCATAACAGATGCGCAAACAGGTTCATCTAAGTGTGTGTGTTC |
| | TGTTATTGATTTATTACTTGATGATTTTGTTGAAATAATAAAATCCCAAGATTTATCTGTAGTTTCT |
| | AAGGTTGTCAAAGTGACTATTGACTATACAGAAATTTCATTTATGCTTTGGTGTAAAGATGGCCATG |
| | TAGAAACATTTTACCCAAAATTACAATCTAGTCAAGCGTGGCAACCGGGTGTTGCTATGCCTAATCT |
| | TTACAAAATGCAAAGAATGCTATTAGAAAATTGTGACCTTCAAAATTATGGTGATAGTGCAACATTA |
| | CCTAAAGGCATAATGATGAATGTCGCAAAATATACTCAACTGTGTCAATATTTAAACACATTAACAT |
| | TAGCTGTACCCTATAATATGAGAGTTATACATTTTGGTGCTGGTTCTGATAAAGGAGTTGCACCAGG |
| | TACAGCTGTTTTAAGACAGTGGTTGCCTACGGGTACGCTGCTTGTCGATTCAGATCTTAATGACTTT |
| | GTCTCTGATGCAGATTCAACTTTGATTGGTGATTGTGCAACTGTACATACAGCTAATAAATGGGATC |
| | TCATTATTAGTGATATGTACGACCCTAAGACTAAAAATGTTACAAAAGAAAATGACTCTAAAGAGGG |
| | TTTTTTCACTTACATTTGTGGGTTTATACAANAAAAGCTAGCTCTTGGAGGTTCCGTGGCTATAAAG |
| | ATAACAGAACATTCTTGGAATGCTGATCTTTATAAGCTCATGGGACACTTCGCATGGTGGACAGCCT |
| | TTGTTACTAATGTGAATGCGTCATCATCTGAAGCATTTTTAATTGGATGTAATTATCTTGGCAAACC |
| | ACGCGAACAAATAGATGGTTATGTCATGCATGCAAATTACATATTTTGGAGGAATACAAATCCAATT |
| | CAGTTGTCTTCCTATTCTTTATTTGACATGAGTAAATTTCCCCTTAAATTAAGGGGTACTGCTGTTA |
| | TGTCTTTAAAAGAAGGTCAAATCAATGATATGATTTTATCTCTTCTTAGTAAAGGTAGACTTATAAT |
| | TAGAGAAAACAACAGAGTTGTTATTTCTAGTGATGTTCTTGTTAACAACTAAACGAACAATGTTTGT |
| | TTTTCTTGTTTTATTGCCACTAGTCTCTAGTCAGTGTGTTAATCTTACAACCAGAACTCAATTACCC |
| | CCTGCATACACTAATTCTTTCACACGTGGTGTTTATTACCCTGACAAAGTTTTCAGATCCTCAGTTT |
| | TACATTCAACTCAGGACTTGTTCTTACCTTTCTTTTCCAATGTTACTTGGTTCCATGCTATCTCTGG |
| | GACCAATGGTACTAAGAGGTTTGATAACCCTGTCCTACCATTTAATGATGGTGTTTATTTTGCTTCC |
| | ACTGAGAAGTCTAACATAATAAGAGGCTGGATTTTTGGTACTACTTTAGATTCGAAGACCCAGTCCC |
| | TACTTATTGTTAATAACGCTACTAATGTTGTTATTAAAGTCTGTGAATTTCAATTTTGTAATGATCC |
| | ATTTTTGGGTGTTTACCACAAAAACAACAAAAGTTGGATGGAAAGTGAGTTCAGAGTTTATTCTAGT |
| | GCGAATAATTGCACTTTTGAATATGTCTCTCAGCCTTTTCTTATGGACCTTGAAGGAAAACAGGGTA |
| | ATTTCAAAAATCTTAGGGAATTTGTGTTTAAGAATATTGATGGTTATTTTAAAATATATTCTAAGCA |
| | CACGCCTATTAATTTAGTGCGTGATCTCCCTCAGGGTTTTTCGGCTTTAGAACCATTGGTAGATTTG |
| | CCAATAGGTATTAACATCACTAGGTTTCAAACTTTACTTGCTTTACATAGAAGTTATTTGACTCCTG |
| | GTGATTCTTCTTCAGGTTGGACAGCTGGTGCTGCAGCTTATTATGTGGGTTATCTTCAACCTAGGAC |
| | TTTTCTATTAAAATATAATGAAAATGGAACCATTACAGATGCTGTAGACTGTGCACTTGACCCTCTC |
| | TCAGAAACAAAGTGTACGTTGAAATCCTTCACTGTAGAAAAAGGAATCTATCAAACTTCTAACTTTA |
| | GAGTCCAACCAACAGAATCTATTGTTAGATTTCCTAATATTACAAACTTGTGCCCTTTTGGTGAAGT |
| | TTTTAACGCCACCAGATTTGCATCTGTTTATGCTTGGAACAGGAAGAGAATCAGCAACTGTGTTGCT |
| | GATTATTCTGTCCTATATAATTCCGCATCATTTTCCACTTTTAAGTGTTATGGAGTGTCTCCTACTA |
| | AATTAAATGATCTCTGCTTTACTAATGTCTATGCAGATTCATTTGTAATTAGAGGTGATGAAGTCAG |
| | ACAAATCGCTCCAGGGCAAACTGGAAAGATTGCTGATTATAATTATAAATTACCAGATGATTTTACA |
| | GGCTGCGTTATAGCTTGGAATTCTAACAATCTTGATTCTAAGGTTGGTGGTAATTATAATTACCTGT |
| | ATAGATTGTTTAGGAAGTCTAATCTCAAACCTTTTGAGAGAGATATTTCAACTGAAATCTATCAGGC |
| | CGGTAGCACACCTTGTAATGGTGTTGAAGGTTTTAATTGTTACTTTCCTTTACAATCATATGGTTTC |
| | CAACCCACTTATGGTGTTGGTTACCAACCATACAGAGTAGTAGTACTTTCTTTTGAACTTCTACATG |
| | CACCAGCAACTGTTTGTGGACCTAAAAAGTCTACTAATTTGGTTAAAAACAAATGTGTCAATTTCAA |
| | CTTCAATGGTTTAACAGGCACAGGTGTTCTTACTGAGTCTAACAAAAAGTTTCTGCCTTTCCAACAA |
| | TTTGGCAGAGACATTGATGACACTACTGATGCTGTCCGTGATCCACAGACACTTGAGATTCTTGACA |
| | TTACACCATGTTCTTTTGGTGGTGTCAGTGTTATAACACCAGGAACAAATACTTCTAACCAGGTTGC |
| | TGTTCTTTATCAGGGTGTTAACTGCACAGAAGTCCCTGTTGCTATTCATGCAGATCAACTTACTCCT |
| | ACTTGGCGTGTTTATTCTACAGGTTCTAATGTTTTTCAAACACGTGCAGGCTGTTTAATAGGGGCTG |
| | AACATGTCAACAACTCATATGAGTGTGACATACCCATTGGTGCAGGTATATGCGCTAGTTATCAGAC |
| | TCAGACTAATTCTCATCGGCGGGCACGTAGTGTAGCTAGTCAATCCATCATTGCCTACACTATGTCA |
| | CTTGGTGCAGAAAATTCAGTTGCTTACTCTAATAACTCTATTGCCATACCCATAAATTTTACTATTA |
| | GTGTTACCACAGAAATTCTACCAGTGTCTATGACCAAGACATCAGTAGATTGTACAATGTACATTTG |
| | TGGTGATTCAACTGAATGCAGCAATCTTTTGTTGCAATATGGCAGTTTTTGTACACAATTAAACCGT |
| | GCTTTAACTGGAATAGCTGTTGAACAAGACAAAAACACCCAAGAAGTTTTTGCACAAGTCAAACAAA |
| | TTTACAAAACACCACCAATTAAAGATTTTGGTGGTTTTAATTTTTCACAAATATTACCAGATCCATC |
| | AAAACCAAGCAAGAGGTCATTTATTGAAGATCTACTTTTCAACAAAGTGACACTTGCAGATGCTGGC |
| | TTCATCAAACAATATGGTGATTGCCTTGGTGATATTGCTGCTAGAGACCTCATTTGTGCACAAAAGT |
| | TTAACGGCCTTACTGTTTTGCCACCTTTGCTCACAGATGAAATGATTGCTCAATACACTTCTGCACT |
| | GTTAGCGGGTACAATCACTTCTGGTTGGACCTTTGGTGCAGGTGCTGCATTACAAATACCATTTGCT |
| | ATGCAAATGGCTTATAGGTTTAATGGTATTGGAGTTACACAGAATGTTCTCTATGAGAACCAAAAAT |
| | TGATTGCCAACCAATTTAATAGTGCTATTGGCAAAATTCAAGACTCACTTTCTTCCACAGCAAGTGC |
| | ACTTGGAAAACTTCAAGATGTGGTCAACCAAAATGCACAAGCTTTAAACACGCTTGTTAAACAACTT |
| | AGCTCCAATTTTGGTGCAATTTCAAGTGTTTTAAATGATATCCTTGCACGTCTTGACAAAGTTGAGG |
| | CTGAAGTGCAAATTGATAGGTTGATCACAGGCAGACTTCAAAGTTTGCAGACATATGTGACTCAACA |
| | ATTAATTAGAGCTGCAGAAATCAGAGCTTCTGCTAATCTTGCTGCTACTAAAATGTCAGAGTGTGTA |
| | CTTGGACAATCAAAAAGAGTTGATTTTTGTGGAAAGGGCTATCATCTTATGTCCTTCCCTCAGTCAG |
| | CACCTCATGGTGTAGTCTTCTTGCATGTGACTTATGTCCCTGCACAAGAAAAGAACTTCACAACTGC |
| | TCCTGCCATTTGTCATGATGGAAAAGCACACTTTCCTCGTGAAGGTGTCTTTGTTTCAAATGGCACA |
| | CACTGGTTTGTAACACAAAGGAATTTTTATGAACCACAAATCATTACTACACACAACACATTTGTGT |
| | CTGGTAACTGTGATGTTGTAATAGGAATTGTCAACAACACAGTTTATGATCCTTTGCAACCTGAATT |
| | AGACTCATTCAAGGAGGAGTTAGATAAATATTTTAAGAATCATACATCACCAGATGTTGATTTAGGT |
| | GACATCTCTGGCATTAATGCTTCAGTTGTAAACATTCAAAAAGAAATTGACCGCCTCAATGAGGTTG |
| | CCAAGAATTTAAATGAATCTCTCATCGATCTCCAAGAACTTGGAAAGTATGAGCAGTATATAAAATG |
| | GCCATGGTACATTTGGCTAGGTTTTATAGCTGGCTTGATTGCCATAGTAATGGTGACAATTATGCTT |
| | TGCTGTATGACCAGTTGCTGTAGTTGTCTCAAGGGCTGTTGTTCTTGTGGATCCTGCTGCAAATTTG |
| | ATGAAGACGACTCTGAGCCAGTGCTCAAAGGAGTCAAATTACATTACACATAAACGAACTTATGGAT |
| | TTGTTTATGAGAATCTTCACAATTGGAACTGTAACTTTGAAGCAAGGTGAAATCAAGGATGCTACTC |
| | CTTCAGATTTTGTTCGCGCTACTGCAACGATACCGATACAAGCCTCACTCCCTTTCGGATGGCTTAT |
| | TGTTGGCGTTGCACTTCTTGCTGTTTTTCAGAGCGCTTCCAAAATCATAACCCTCAAAAAGAGATGG |
| | CAACTAGCACTCTCCAAGGGTGTTCACTTTGTTTGCAACTTGCTGTTGTTGTTTGTAACAGTTTACT |
| | CACACCTTTTGCTCGTTGCTGCTGGCCTTGAAGCCCCTTTTCTCTATCTTTATGCTTTAGTCTACTT |
| | CTTGCAGAGTATAAACTTTGTAAGAATAATAATGAGGCTTTGGCTTTGCTGTAAATGCCGTTCCAAA |
| | AACCCATTACTTTATGATGCCAACTATTTTCTTTGCTGGCATACTAATTGTTACGACTATTGTATAC |
| | CTTACAATAGTGTAACTTCTTCAATTGTCATTACTTCAGGTGATGGCACAACAAGTCCTATTTCTGA |
| | ACATGACTACCAGATTGGTGGTTATACTGAAAAATGGGAATCTGGAGTAAAAGACTGTGTTGTATTA |
| | CACAGTTACTTCACTTCAGACTATTACCAGCTGTACTCAACTCAATTGAGTACAGACACTGGTGTTG |
| | AACATGTTACCTTCTTCATCTACAATAAAATTGTTGATGAGCCTGAAGAACATGTCCAAATTCACAC |
| | AATCGACGGTTCATCCGGAGTTGTTAATCCAGTAATGGAACCAATTTATGATGAACCGACGACGACT |
| | ACTAGCGTGCCTTTGTAAGCACAAGCTGATGAGTACGAACTTATGTACTCATTCGTTTCGGAAGAGA |
| | CAGGTACGTTAATAGTTAATAGCGTACTTCTTTTTCTTGCTTTCGTGGTATTCTTGCTAGTTACACT |
| | AGCCATCCTTACTGCGCTTCGATTGTGTGCGTACTGCTGCAATATTGTTAACGTGAGTCTTGTAAAA |
| | CCTTCTTTTTACGTTTACTCTCGTGTTAAAAATCTGAATTCTTCTAGAGTTCCTGATCTTCTGGTCT |
| | AAACGAACTAAATATTATATTAGTTTTTCTGTTTGGAACTTTAATTTTAGCCATGGCAGATTCCAAC |
| | GGTACTATTACCGTTGAAGAGCTTAAAAAGCTCCTTGAACAATGGAACCTAGTAATAGGTTTCCTAT |
| | TCCTTACATGGATTTGTCTTCTACAATTTGCCTATGCCAACAGGAATAGGTTTTTGTATATAATTAA |
| | GTTAATTTTCCTCTGGCTGTTATGGCCAGTAACTTTAGCTTGTTTTGTGCTTGCTGCTGTTTACAGA |
| | ATAAATTGGATCACCGGTGGAATTGCTATCGCAATGGCTTGTCTTGTAGGCTTGATGTGGCTCAGCT |
| | ACTTCATTGCTTCTTTCAGACTGTTTGCGCGTACGCGTTCCATGTGGTCATTCAATCCAGAAACTAA |
| | CATTCTTCTCAACGTGCCACTCCATGGCACTATTCTGACCAGACCGCTTCTAGAAAGTGAACTCGTA |
| | ATCGGAGCTGTGATCCTTCGTGGACATCTTCGTATTGCTGGACACCATCTAGGACGCTGTGACATCA |
| | AGGACCTGCCTAAAGAAATCACTGTTGCTACATCACGAACGCTTTCTTATTACAAATTGGGAGCTTC |
| | GCAGCGTGTAGCAGGTGACTCAGGTTTTGCTGCATACAGTCGCTACAGGATTGGCAACTATAAATTA |
| | AACACAGACCATTCCAGTAGCAGTGACAATATTGCTTTGCTTGTACAGTAAGTGACAACAGATGTTT |
| | CATCTCGTTGACTTTCAGGTTACTATAGCAGAGATATTACTAATTATTATGAGGACTTTTAAAGTTT |
| | CCATTTGGAATCTTGATTACATCATAAACCTCATAATTAAAAATTTATCTAAGTCACTAACTGAGAA |
| | TAAATATTCTCAATTAGATGAAGAGCAACCAATGGAGATTGATTAAACGAACATGAAAATTATTCTT |
| | TTCTTGGCACTGATAACACTCGCTACTTGTGAGCTTTATCACTACCAAGAGTGTGTTAGAGGTACAA |
| | CAGTACTTTTAAAAGAACCTTGCTCTTCTGGAACATNNNNNNNNNNNNNNNNNNNNNNNNNNNNNNN |
| | NNNNNNNNNNNNNNNNNNNNNNNNNNNNNNNNNNNNNNNNNNNNNNNNNNNNNNNNNNNNNNNNNNN |
| | NNNNNNNNNNNNNNNNNNNNNNNNNNNNNNNNNNNNNNNNNNNNNNNNNNNNNNNNNNNNNNNNNNN |
| | NNNNNNNNNNNNNNNNNNNNNNNNNNNNNNNNNNNNNNNNNNNNNNNNNNNNNNNNNNNNNNNNNNN |
| | NNNNNNNNNNNNNNNNNNNNNNNNNNNNNNNNNNNNNNNNNNNNNNNNNNNNNNNNNNNNNNNNNAT |
| | TCCTTGTTTTAATTATGCTTATTATCTTTTGGTTCTCACTTGAACTGCAAGATCATAATGAAACTTG |
| | TCACGCCTAAACGAACATGAAATTTCTTGTTTTCTTAGGAATCATCACAACTGTAGCTGCATTTCAC |
| | CAAGAATGTAGTTTACAGTCATGTACTTAACATCAACCATATGTAGTTGATGACCCGTGTCCTATTC |
| | ACTTCTATTCTAAATGGTATATTAGAGTAGGAGCTATAAAATCAGCACCTTTAATTGAATTGTGCGT |
| | GGATGAGGCTGGTTCTTAATCACCCATTCAGTGCATCGATATCGGTAATTATACAGTTTCCTGTTTA |
| | CCTTTTACAATTAATTGCCAGGAACCTAAATTGGGTAGTCTTGTAGTGCGTTGTTCGTTCTATGAAG |
| | ACTTTTTAGAGTATCATGACGTTCGTGTTGTTTTAGATTTCATCTAAACGAACAAACTAAATGTCTC |
| | TAAATGGACCCCAAAATCAGCGAAATGCACCCCGCATTACGTTTGGTGGACCCTCAGATTCAACTGG |
| | CAGTAACCAGAATGGAGAACGCAGTGGGGCGCGATCAAAACAACGTCGGCCCCAAGGTTTACCCAAT |
| | AATACTGCGTCTTGGTTCACCGCTCTCACTCAACATGGCAAGGAAGACCTTAAATTCCCTCGAGGAC |
| | AAGGCGTTCCAATTAACACCAATAGCAGTCCAGATGACCAAATTGGCTACTACCGAAGAGCTACCAG |
| | ACGAATTCGTGGTGGTGACGGTAAAATGAAAGATCTCAGTCCAAGATGGTATTTCTACTACCTAGGA |
| | ACTGGGCCAGAAGCTGGACTTCCCTATGGTGCTAACAAAGACGGCATCATATGGGTTGCAACTGAGG |
| | GAGCCTTGAATACACCAAAAGATCACATTGGCACCCGCAATCCTGCTAACAATGCTGCAATCGTGCT |
| | ACAACTTCCTCAAGGAACAACATTGCCAAAAGGCTTCTACGCAGAAGGGAGCAGAGGCGGCAGTCAA |
| | GCCTCTTCTCGTTCCTCATCACGTAGTCGCAACAGTTCAAGAAATTCAACTCCAGGCAGCAGTAAAC |
| | GAACTTCTCCTGCTAGAATGGCTGGCAATGGCGGTGATGCTGCTCTTGCTTTGCTGCTGCTTGACAG |
| | ATTGAACCAGCTTGAGAGCAAAATGTTTGGTAAAGGCCAACAACAACAAGGCCAAACTGTCACTAAG |
| | AAATCTGCTGCTGAGGCTTCTAAGAAGCCTCGGCAAAAACGTACTGCCACTAAAGCATACAATGTAA |
| | CACAAGCTTTCGGCAGACGTGGTCCAGAACAAACCCAAGGAAATTTTGGGGACCAGGAACTAATCAG |
| | ACAAGGAACTGATTACAAACATTGGCCGCAAATTGCACAATTTGCCCCCAGCGCTTCAGCGTTCTTC |
| | GGAATGTCGCGCATTGGCATGGAAGTCACACCTTCGGGAACGTGGTTGACCTACACAGGTGCCATCA |
| | AATTGGATGACAAAGATCCAAATTTCAAAGATCAAGTCATTTTGCTGAATAAGCATATTGACGCATA |
| | CAAAACATTCCCACCAACAGAGCCTAAAAAGGACAAAAAGAAGAAGGCTGATGAAACTCAAGCCTTA |
| | CCGCAGAGACAGAAGAAACAGCAAACTGTGACTCTTCTTCCTGCTGCAGATTTGGATGATTTCTCCA |
| | AACAATTGCAACAATCCATGAGCAGTGCTGACTCAACTCAGGCCTAAACTCATGCAGACCACACAAG |
| | GCAGATGGGCTATATAAACGTTTTCGCTTTTCCGTTTACGATATATAGTCTACTCTTGTGCAGAATG |
| | AATTCTCGTAACTACATAGCACAAGTAGATGTAGTTAACTTTAATCTCACATAGCAATCTTTAATCA |
| | GTGTGTAACATTAGGGAGGACTTGAAAGAGCCACCACATTTTCACCGAGGCCACGCGGAGTACGATC |
| | GAGTGTACAATGAACAATGCTAGGGAGAGCTGCCTATATGGAAGAGCCCTAATGTGTAAAATTAATT |
| | TTAGTAGTGCTATCC |
| 292 | GenBank Accession No. MW531680.1 (B.1.1.7 SARS-CoV-2 variant; Severe |
| | acute respiratory syndrome coronavirus 2 isolate SARS-CoV- |
| | 2/human/BGD/G039392/2021, complete genome) |
| | TGTAGATCTGTTCTCTAAACGAACTTTAAAATCTGTGTGGCTGTCACTCGGCTGCATGCTTAGTGCA |
| | CTCACGCAGTATAATTAATAACTAATTACTGTCGTTGACAGGACACGAGTAACTCGTCTATCTTCTG |
| | CAGGCTGCTTACGGTTTCGTCCGTGTTGCAGCCGATCATCAGCACATCTAGGTTTTGTCCGGGTGTG |
| | ACCGAAAGGTAAGATGGAGAGCCTTGTCCCTGGTTTCAACGAGAAAACACACGTCCAACTCAGTTTG |
| | CCTGTTTTACAGGTTCGCGACGTGCTCGTACGTGGCTTTGGAGACTCCGTGGAGGAGGTCTTATCAG |
| | AGGCACGTCAACATCTTAAAGATGGCACTTGTGGCTTAGTAGAAGTTGAAAAAGGCGTTTTGCCTCA |
| | ACTTGAACAGCCCTATGTGTTCATCAAACGTTCGGATGCTCGAACTGCACCTCATGGTCATGTTATG |
| | GTTGAGCTGGTAGCAGAACTCGAAGGCATTCAGTACGGTCGTAGTGGTGAGACACTTGGTGTCCTTG |
| | TCCCTCATGTGGGCGAAATACCAGTGGCTTACCGCAAGGTTCTTCTTCGTAAGAACGGTAATAAAGG |
| | AGCTGGTGGCCATAGTTACGGCGCCGATCTAAAGTCATTTGACTTAGGCGACGAGCTTGGCACTGAT |
| | CCTTATGAAGATTTTCAAGAAAACTGGAACACTAAACATAGCAGTGGTGTTACCCGTGAACTCATGC |
| | GTGAGCTTAACGGAGGGGCATACACTCGCTATGTCGATAACAACTTCTGTGGCCCTGATGGCTACCC |
| | TCTTGAGTGCATTAAAGACCTTCTAGCACGTGCTGGTAAAGCTTCATGCACTTTGTCTGAACAACTG |
| | GACTTTATTGACACTAAGAGGGGTGTATACTGCTGCCGTGAACATGAGCATGAAATTGCTTGGTACA |
| | CGGAACGTTCTGAAAAGAGCTATGAATTGCAGACACCTTTTGAAATTAAATTGGCAAAGAAATTTGA |
| | CACCTTCAATGGGGAATGTCCAAATTTTGTATTTCCCTTAAATTCCATAATCAAGACTATTCAACCA |
| | AGGGTTGAAAAGAAAAAGCTTGATGGCTTTATGGGTAGAATTCGATCTGTCTATCCAGTTGCGTCAC |
| | CAAATGAATGCAACCAAATGTGCCTTTCAACTCTCATGAAGTGTGATCATTGTGGTGAAACTTCATG |
| | GCAGACGGGCGATTTTGTTAAAGCCACTTGCGAATTTTGTGGCACTGAGAATTTGACTAAAGAAGGT |
| | GCCACTACTTGTGGTTACTTACCCCAAAATGCTGTTGTTAAAATTTATTGTCCAGCATGTCACAATT |
| | CAGAAGTAGGACCTGAGCATAGTCTTGCCGAATACCATAATGAATCTGGCTTGAAAACCATTCTTCG |
| | TAAGGGTGGTCGCACTATTGCCTTTGGAGGCTGTGTGTTCTCTTATGTTGGTTGCCATAACAAGTGT |
| | GCCTATTGGGTTCCACGTGCTAGCGCTAACATAGGTTGTAACCATACAGGTGTTGTTGGAGAAGGTT |
| | CCGAAGGTCTTAATGACAACCTTCTTGAAATACTCCAAAAAGAGAAAGTCAACATCAATATTGTTGG |
| | TGACTTTAAACTTAATGAAGAGATCGCCATTATTTTGGCATCTTTTTCTGCTTCCACAAGTGCTTTT |
| | GTGGAAACTGTGAAAGGTTTGGATTATAAAGCATTCAAACAAATTGTTGAATCCTGTGGTAATTTTA |
| | AAGTTACAAAAGGAAAAGCTAAAAAAGGTGCCTGGAATATTGGTGAACAGAAATCAATACTGAGTCC |
| | TCTTTATGCATTTGCATCAGAGGCTGCTCGTGTTGTACGATCAATTTTCTCCCGCACTCTTGAAACT |
| | GCTCAAAATTCTGTGCGTGTTTTACAGAAGGCCGCTATAACAATACTAGATGGAATTTCACAGTATT |
| | CACTGAGACTCATTGATGCTATGATGTTCACATCTGATTTGGCTACTAACAATCTAGTTGTAATGGC |
| | CTACATTACAGGTGGTGTTGTTCAGTTGACTTCGCAGTGGCTAACTAACATCTTTGGCACTGTTTAT |
| | GAAAAACTCAAACCCGTCCTTGATTGGCTTGAAGAGAAGTTTAAGGAAGGTGTAGAGTTTCTTAGAG |
| | ACGGTTGGGAAATTGTTAAATTTATCTCAACCTGTGCTTGTGAAATTGTCGGTGGACAAATTGTCAC |
| | CTGTGCAAAGGAAATTAAGGAGAGTGTTCAGACATTCTTTAAGCTTGTAAATAAATTTTTGGCTTTG |
| | TGTGCTGACTCTATCATTATTGGTGGAGCTAAACTTAAAGCCTTGAATTTAGGTGAAACATTTGTCA |
| | CGCACTCAAAGGGATTGTACAGAAAGTGTGTTAAATCCAGAGAAGAAACTGGCCTACTCATGCCTCT |
| | AAAAGCCCCAAAAGAAATTATCTTCTTAGAGGGAGAAACACTTCCCACAGAAGTGTTAACAGAGGAA |
| | GTTGTCTTGAAAACTGGTGATTTACAACCATTAGAACAACCTACTAGTGAAGCTGTTGAAGCTCCAT |
| | TGGTTGGTACACCAGTTTGTATTAACGGGCTTATGTTGCTCGAAATCAAAGACACAGAAAAGTACTG |
| | TGCCCTTGCACCTAATATGATGGTAACAAACAATACCTTCACACTCAAAGGCGGTGCACCAACAAAG |
| | GTTACTTTTGGTGATGACACTGTGATAGAAGTGCAAGGTTACAAGAGTGTGAATATCACTTTTGAAC |
| | TTGATGAAAGGATTGATAAAGTACTTAATGAGAAGTGCTCTGCCTATACAGTTGAACTCGGTACAGA |
| | AGTAAATGAGTTCGCCTGTGTTGTGGCAGATGCTGTCATAAAAACTTTGCAACCAGTATCTGAATTA |
| | CTTACACCACTGGGCATTGATTTAGATGAGTGGAGTATGGCTACATACTACTTATTTGATGAGTCTG |
| | GTGAGTTTAAATTGGCTTCACATATGTATTGTTCTTTTTACCCTCCAGATGAGGATGAAGAAGAAGG |
| | TGATTGTGAAGAAGAAGAGTTTGAGCCATCAACTCAATATGAGTATGGTACTGAAGATGATTAGCAA |
| | GGTAAACCTTTGGAATTTGGTGCCACTTCTGCTGCTCTTCAACCTGAAGAAGAGCAAGAAGAAGATT |
| | GGTTAGATGATGATAGTCAACAAACTGTTGGTCAACAAGACGGCAGTGAGGACAATCAGACAACTAT |
| | TATTCAAACAATTGTTGAGGTTCAACCTCAATTAGAGATGGAACTTACACCAGTTGTTCAGACTATT |
| | GAAGTGAATAGTTTTAGTGGTTATTTAAAACTTACTGACAATGTATACATTAAAAATGCAGACATTG |
| | TGGAAGAAGCTAAAAAGGTAAAACCAACAGTGGTTGTTAATGCAGCCAATGTTTACCTTAAACATGG |
| | AGGAGGTGTTGCAGGAGCCTTAAATAAGGCTACTAACAATGCCATGCAAGTTGAATCTGATGATTAC |
| | ATAGCTACTAATGGACCACTTAAAGTGGGTGGTAGTTGTGTTTTAAGCGGACACAATCTTGCTAAAC |
| | ACTGTCTTCATGTTGTCGGCCCAAATGTTAACAAAGGTGAAGACATTCAACTTCTTAAGAGTGCTTA |
| | TGAAAATTTTAATCAGCACGAAGTTCTACTTGCACCATTATTATCAGCTGGTATTTTTGGTGCTGAC |
| | CCTATACATTCTTTAAGAGTTTGTGTAGATACTGTTCGCACAAATGTCTACTTAGCTGTCTTTGATA |
| | AAAATCTCTATGACAAACTTGTTTCAAGCTTTTTGGAAATGAAGAGTGAAAAGCAAGTTGAACAAAA |
| | GATCGCTGAGATTCCTAAAGAGGAAGTTAAGCCATTTATAACTGAAAGTAAACCTTCAGTTGAACAG |
| | AGAAAACAAGATGATAAGAAAATCAAAGCTTGTGTTGAAGAAGTTACAACAACTCTGGAAGAAACTA |
| | AGTTCCTCACAGAAAACTTGTTACTTTATATTGACATTAATGGCAATCTTCATCCAGATTCTGCCAC |
| | TCTTGTTAGTGACATTGACATCACTTTCTTAAAGAAAGATGCTCCATATATAGTGGGTGATGTTGTT |
| | CAAGAGGGTGTTTTAACTGCTGTGGTTATACCTACTAAAAAGGCTGGTGGCACTACTGAAATGCTAG |
| | CGAAAGCTTTGAGAAAAGTGCCAACAGACAATTATATAACCACTTACCCGGGTCAGGGTTTAAATGG |
| | TTACACTGTAGAGGAGGCAAAGACAGTGCTTAAAAAGTGTAAAAGTGCCTTTTACATTCTACCATCT |
| | ATTATCTCTAATGAGAAGCAAGAAATTCTTGGAACTGTTTCTTGGAATTTGCGAGAAATGCTTGCAC |
| | ATGCAGAAGAAACACGCAAATTAATGCCTGTCTGTGTGGAAACTAAAGCCATAGTTTCAACTATACA |
| | GCGTAAATATAAGGGTATTAAAATACAAGAGGGTGTGGTTGATTATGGTGCTAGATTTTACTTTTAC |
| | ACCAGTAAAACAACTGTAGCGTCACTTATCAACACACTTAACGATCTAAATGAAACTCTTGTTACAA |
| | TGCCACTTGGCTATGTAACACATGGCTTAAATTTGGAAGAAGCTGCTCGGTATATGAGATCTCTCAA |
| | AGTGCCAGCTACAGTTTCTGTTTCTTCACCTGATGCTGTTACAGCGTATAATGGTTATCTTACTTCT |
| | TCTTCTAAAACACCTGAAGAACATTTTATTGAAACCATCTCACTTGCTGGTTCCTATAAAGATTGGT |
| | CCTATTCTGGACAATCTACACAACTAGGTATAGAATTTCTTAAGAGAGGTGATAAAAGTGTATATTA |
| | CACTAGTAATCCTACCACATTCCACCTAGATGGTGAAGTTATCACCTTTGACAATCTTAAGACACTT |
| | CTTTCTTTGAGAGAAGTGAGGACTATTAAGGTGTTTACAACAGTAGACAACATTAACCTCCACACGC |
| | AAGTTGTGGACATGTCAATGACATATGGACAACAGTTTGGTCCAACTTATTTGGATGGAGCTGATGT |
| | TACTAAAATAAAACCTCATAATTCACATGAAGGTAAAACATTTTATGTTTTACCTAATGATGACACT |
| | CTACGTGTTGAGGCTTTTGAGTACTACCACACAACTGATCCTAGTTTTCTGGGTAGGTACATGTCAG |
| | CATTAAATCACACTAAAAAGTGGAAATACCCACAAGTTAATGGTTTAACTTCTATAAAATGGGCAGA |
| | TAACAACTGTTATCTTGCCACTGCATTGTTAACACTCCAACAAATAGAGTTGAAGTTTAATCCACCT |
| | GCTCTACAAGATGCTTATTACAGAGCAAGGGCTGGTGAAGCTGATAACTTTTGTGCACTTATCTTAG |
| | CCTACTGTAATAAGACAGTAGGTGAGTTAGGTGATGTTAGAGAAACAATGAGTTACTTGTTTCAACA |
| | TGCCAATTTAGATTCTTGCAAAAGAGTCTTGAACGTGGTGTGTAAAACTTGTGGACAACAGCAGACA |
| | ACCCTTAAGGGTGTAGAAGCTGTTATGTACATGGGCACACTTTCTTATGAACAATTTAAGAAAGGTG |
| | TTCAGATACCTTGTACGTGTGGTAAACAAGCTACAAAATATCTAGTACAACAGGAGTCACCTTTTGT |
| | TATGATGTCAGCACCACCTGCTCAGTATGAACTTAAGCATGGTACATTTACTTGTGCTAGTGAGTAC |
| | ACTGGTAATTACCAGTGTGGTCACTATAAACATATAACTTCTAAAGAAACTTTGTATTGCATAGACG |
| | GTGCTTTACTTACAAAGTCCTCAGAATACAAAGGTCCTATTACGGATGTTTTCTACAAAGAAAACAG |
| | TTACACAACAACCATAAAACCAGTTACTTATAAATTGGATGGTGTTGTTTGTACAGAAATTGACCCT |
| | AAGTTGGACAATTATTATAAGAAAGACAATTCTTATTTTACAGAGCAACCAATTGATCTTGTACCAA |
| | ACCAACCATATCCAAACGCAAGCTTCGATAATTTTAAGTTTGTATGTGATAATATCAAATTTGCTGA |
| | TGATTTAAACCAGTTAACTGGTTATAAGAAACCTGCTTCAAGAGAGCTTAAAGTTACATTTTTCCCT |
| | GACTTAAATGGTGATGTGGTGGCTATTGATTATAAACACTACACACCCTCTTTTAAGAAAGGAGCTA |
| | AATTGTTACATAAACCTATTGTTTGGCATGTTAACAATGCAACTAATAAAGCCACGTATAAACCAAA |
| | TACCTGGTGTATACGTTGTCTTTGGAGCACAAAACCAGTTGAAACATCAAATTCGTTTGATGTACTG |
| | AAGTCAGAGGACGCGCAGGGAATGGATAATCTTGCCTGCGAAGATCTAAAACCAGTCTCTGAAGAAG |
| | TAGTGGAAAATCCTACCATACAGAAAGACGTTCTTGAGTGTAATGTGAAAACTACCGAAGTTGTAGG |
| | AGACATTATACTTAAACCAGCAAATAATAGTTTAAAAATTACAGAAGAGGTTGGCCACACAGATCTA |
| | ATGGCTGCTTATGTAGACAATTCTAGTCTTACTATTAAGAAACCTAATGAATTATCTAGAGTATTAG |
| | GTTTGAAAACCCTTGCTACTCATGGTTTAGCTGCTGTTAATAGTGTCCCTTGGGATACTATAGCTAA |
| | TTATGCTAAGCCTTTTCTTAACAAAGTTGTTAGTACAACTACTAACATAGTTACACGGTGTTTAAAC |
| | CGTGTTTGTACTAATTATATGCCTTATTTCTTTACTTTATTGCTACAATTGTGTACTTTTACTAGAA |
| | GTACAAATTCTAGAATTAAAGCATCTATGCCGACTACTATAGCAAAGAATACTGTTAAGAGTGTCGG |
| | TAAATTTTGTCTAGAGGCTTCATTTAATTATTTGAAGTCACCTAATTTTTCTAAACTGATAAATATT |
| | ACAATTTGGTTTTTACTATTAAGTGTTTGCCTAGGTTCTTTAATCTACTCAACCGCTGCTTTAGGTG |
| | TTTTAATGTCTAATTTAGGCATGCCTTCTTACTGTACTGGTTACAGAGAAGGCTATTTGAACTCTAC |
| | TAATGTCACTATTGCAACCTACTGTACTGGTTCTATACCTTGTAGTGTTTGTCTTAGTGGTTTAGAT |
| | TCTTTAGACACCTATCCTTCTTTAGAAACTATACAAATTACCATTTCATCTTTTAAATGGGATTTAA |
| | CTGCTTTTGGCTTAGTTGCAGAGTGGTTTTTGGCATATATTCTTTTCACTAGGTTTTTCTATGTACT |
| | TGGATTGGCTGCAATCATGCAATTGTTTTTCAGCTATTTTGCAGTACATTTTATTAGTAATTCTTGG |
| | CTTATGTGGTTAATAATTAATCTTGTACAAATGGCCCCGATTTCAGCTATGGTTAGAATGTACATCT |
| | TCTTTGCATCATTTTATTATGTATGGAAAAGTTATGTGCATGTTGTAGACGGTTGTAATTCATCAAC |
| | TTGTATGATGTGTTACAAACGTAATAGAGCAACAAGAGTCGAATGTACAACTATTGTTAATGGTGTT |
| | AGAAGGTCCTTTTATGTCTATGCTAATGGAGGTAAAGGCTTTTGCAAACTACACAATTGGAATTGTG |
| | TTAATTGTGATACATTCTGTGCTGGTAGTACATTTATTAGTGATGAAGTTGCGAGAGACTTGTCACT |
| | ACAGTTTAAAAGACCAATAAATCCTACTGACCAGTCTTCTTACATCGTTGATAGTGTTACAGTGAAG |
| | AATGGTTCCATCCATCTTTACTTTGATAAAGCTGGTCAAAAGACTTATGAAAGACATTCTCTCTCTC |
| | ATTTTGTTAACTTAGACAACCTGAGAGCTAATAACACTAAAGGTTCATTGCCTATTAATGTTATAGT |
| | TTTTGATGGTAAATCAAAATGTGAAGAATCATCTGCAAAATCAGCGTCTGTTTACTACAGTCAGCTT |
| | ATGTGTCAACCTATACTGTTACTAGATCAGGCATTAGTGTCTGATGTTGGTGATAGTGCGGAAGTTG |
| | CAGTTAAAATGTTTGATGCTTACGTTAATACGTTTTCATCAACTTTTAACGTACCAATGGAAAAACT |
| | CAAAACACTAGTTGCAACTGCAGAAGCTGAACTTGCAAAGAATGTGTCCTTAGACAATGTCTTATCT |
| | ACTTTTATTTCAGCAGCTCGGCAAGGGTTTGTTGATTCAGATGTAGAAACTAAAGATGTTGTTGAAT |
| | GTCTTAAATTGTCACATCAATCTGACATAGAAGTTACTGGCGATAGTTGTAATAACTATATGCTCAC |
| | CTATAACAAAGTTGAAAACATGACACCCCGTGACCTTGGTGCTTGTATTGACTGTAGTGCGCGTCAT |
| | ATTAATGCGCAGGTAGCAAAAAGTCACAACATTGCTTTGATATGGAACGTTAAAGATTTCATGTCAT |
| | TGTCTGAACAACTACGAAAACAAATACGTAGTGCTGCTAAAAAGAATAACTTACCTTTTAAGTTGAC |
| | ATGTGCAACTACTAGACAAGTTGTTAATGTTGTAACAACAAAGATAGCACTTAAGGGTGGTAAAATT |
| | GTTAATAATTGGTTGAAGCAGTTAATTAAAGTTACACTTGTGTTCCTTTTTGTTGCTGCTATTTTCT |
| | ATTTAATAACACCTGTTCATGTCATGTCTAAACATACTGACTTTTCAAGTGAAATCATAGGATACAA |
| | GGCTATTGATGGTGGTGTCACTCGTGACATAGCATCTACAGATACTTGTTTTGCTAACAAACATGCT |
| | GATTTTGACACATGGTTTAGCCAGCGTGGTGGTAGTTATACTAATGACAAAGCTTGCCCATTGATTG |
| | CTGCAGTCATAACAAGAGAAGTGGGTTTTGTCGTGCCTGGTTTGCCTGGCACGATATTACGCACAAC |
| | TAATGGTGACTTTTTGCATTTCTTACCTAGAGTTTTTAGTGCAGTTGGTAACATCTGTTACACACCA |
| | TCAAAACTTATAGAGTACACTGACTTTGCAACATCAGCTTGTGTTTTGGCTGCTGAATGTACAATTT |
| | TTAAAGATGCTTCTGGTAAGCCAGTACCATATTGTTATGATACCAATGTACTAGAAGGTTCTGTTGC |
| | TTATGAAAGTTTACGCCCTGACACACGTTATGTGCTCATGGATGGCTCTATTATTCAATTTCCTAAC |
| | ACCTACCTTGAAGGTTCTGTTAGAGTGGTAACAACTTTTGATTCTGAGTACTGTAGGCACGGCACTT |
| | GTGAAAGATCAGAAGCTGGTGTTTGTGTATCTACTAGTGGTAGATGGGTACTTAACAATGATTATTA |
| | CAGATCTTTACCAGGAGTTTTCTGTGGTGTAGATGCTGTAAATTTACTTACTAATATGTTTACACCA |
| | CTAATTCAACCTATTGGTGCTTTGGACATATCAGCATCTATAGTAGCTGGTGGTATTGTAGCTATCG |
| | TAGTAACATGCCTTGCCTACTATTTTATGAGGTTTAGAAGAGCTTTTGGTGAATACAGTCATGTAGT |
| | TGCCTTTAATACTTTACTATTCCTTATGTCATTCACTGTACTCTGTTTAACACCAGTTTACTCATTC |
| | TTACCTGGTGTTTATTCTGTTATTTACTTGTACTTGACATTTTATCTTACTAATGATGTTTCTTTTT |
| | TAGCACATATTCAGTGGATGGTTATGTTCACACCTTTAGTACCTTTCTGGATAACAATTGCTTATAT |
| | CATTTGTATTTCCACAAAGCATTTCTATTGGTTCTTTAGTAATTACCTAAAGAGACGTGTAGTCTTT |
| | AATGGTGTTTCCTTTAGTACTTTTGAAGAAGCTGCGCTGTGCACCTTTTTGTTAAATAAAGAAATGT |
| | ATCTAAAGTTGCGTAGTGATGTGCTATTACCTCTTACGCAATATAATAGATACTTAGCTCTTTATAA |
| | TAAGTACAAGTATTTTAGTGGAGCAATGGATACAACTAGCTACAGAGAAGCTGCTTGTTGTCATCTC |
| | GCAAAGGCTCTCAATGACTTCAGTAACTCAGGTTCTGATGTTCTTTACCAACCACCACAAACCTCTA |
| | TCACCTCAGCTGTTTTGCAGAGTGGTTTTAGAAAAATGGCATTCCCATCTGGTAAAGTTGAGGGTTG |
| | TATGGTACAAGTAACTTGTGGTACAACTACACTTAACGGTCTTTGGCTTGATGACGTAGTTTACTGT |
| | CCAAGACATGTGATCTGCACCTCTGAAGACATGCTTAACCCTAATTATGAAGATTTACTCATTCGTA |
| | AGTCTAATCATAATTTCTTGGTACAGGCTGGTAATGTTCAACTCAGGGTTATTGGACATTCTATGCA |
| | AAATTGTGTACTTAAGCTTAAGGTTGATACAGCCAATCCTAAGACACCTAAGTATAAGTTTGTTCGC |
| | ATTCAACCAGGACAGACTTTTTCAGTGTTAGCTTGTTACAATGGTTCACCATCTGGTGTTTACCAAT |
| | GTGCTATGAGGCCCAATTTCACTATTAAGGGTTCATTCCTTAATGGTTCATGTGGTAGTGTTGGTTT |
| | TAACATAGATTATGACTGTGTCTCTTTTTGTTACATGCACCATATGGAATTACCAACTGGAGTTCAT |
| | GCTGGCACAGACTTAGAAGGTAACTTTTATGGACCTTTTGTTGACAGGCAAACAGCACAAGCAGCTG |
| | GTACGGACACAACTATTACAGTTAATGTTTTAGCTTGGTTGTACGCTGCTGTTATAAATGGAGACAG |
| | GTGGTTTCTCAATCGATTTACCACAACTCTTAATGACTTTAACCTTGTGGCTATGAAGTACAATTAT |
| | GAACCTCTAACACAAGACCATGTTGACATACTAGGACCTCTTTCTGCTCAAACTGGAATTGCCGTTT |
| | TAGATATGTGTGCTTCATTAAAAGAATTACTGCAAAATGGTATGAATGGACGTACCATATTGGGTAG |
| | TGCTTTATTAGAAGATGAATTTACACCTTTTGATGTTGTTAGACAATGCTCAGGTGTTACTTTCCAA |
| | AGTGCAGTGAAAAGAACAATCAAGGGTACACACCACTGGTTGTTACTCACAATTTTGACTTCACTTT |
| | TAGTTTTAGTCCAGAGTACTCAATGGTCTTTGTTCTTTTTTTTGTATGAAAATGCCTTTTTACCTTT |
| | TGCTATGGGTATTATTGCTATGTCTGCTTTTGCAATGATGTTTGTCAAACATAAGCATGCATTTCTC |
| | TGTTTGTTTTTGTTACCTTCTCTTGCCACTGTAGCTTATTTTAATATGGTCTATATGCCTGCTAGTT |
| | GGGTGATGCGTATTATGACATGGTTGGATATGGTTGATACTAGTTTGAAGCTAAAAGACTGTGTTAT |
| | GTATGCATCAGCTGTAGTGTTACTAATCCTTATGACAGCAAGAACTGTGTATGATGATGGTGCTAGG |
| | AGAGTGTGGACACTTATGAATGTCTTGACACTCGTTTATAAAGTTTATTATGGTAATGCTTTAGATC |
| | AAGCCATTTCCATGTGGGCTCTTATAATCTCTGTTACTTCTAACTACTCAGGTGTAGTTACAACTGT |
| | CATGTTTTTGGCCAGAGGTATTGTTTTTATGTGTGTTGAGTATTGCCCTATTTTCTTCATAACTGGT |
| | AATACACTTCAGTGTATAATGCTAGTTTATTGTTTCTTAGGCTATTTTTGTACTTGTTACTTTGGCC |
| | TCTTTTGTTTACTCAACCGCTACTTTAGACTGACTCTTGGTGTTTATGATTACTTAGTTTCTACACA |
| | GGAGTTTAGATATATGAATTCACAGGGACTACTCCCACCCAAGAATAGCATAGATGCCTTCAAACTC |
| | AACATTAAATTGTTGGGTGTTGGTGGCAAACCTTGTATCAAAGTAGCCACTGTACAGTCTAAAATGT |
| | CAGATGTAAAGTGCACATCAGTAGTCTTACTCTCAGTTTTGCAACAACTCAGAGTAGAATCATCATC |
| | TAAATTGTGGGCTCAATGTGTCCAGTTACACAATGACATTCTCTTAGCTAAAGATACTACTGAAGCC |
| | TTTGAAAAAATGGTTTCACTACTTTCTGTTTTGCTTTCCATGCAGGGTGCTGTAGACATAAACAAGC |
| | TTTGTGAAGAAATGCTGGACAACAGGGCAACCTTACAAGCTATAGCCTCAGAGTTTAGTTCCCTTCC |
| | ATCATATGCAGCTTTTGCTACTGCTCAAGAAGCTTATGAGCAGGCTGTTGCTAATGGTGATTCTGAA |
| | GTTGTTCTTAAAAAGTTGAAGAAGTCTTTGAATGTGGCTAAATCTGAATTTGACCGTGATGCAGCCA |
| | TGCAACGTAAGTTGGAAAAGATGGCTGATCAAGCTATGACCCAAATGTATAAACAGGCTAGATCTGA |
| | GGACAAGAGGGCAAAAGTTACTAGTGCTATGCAGACAATGCTTTTCACTATGCTTAGAAAGTTGGAT |
| | AATGATGCACTCAACAACATTATCAACAATGCAAGAGATGGTTGTGTTCCCTTGAACATAATACCTC |
| | TTACAACAGCAGCCAAACTAATGGTTGTCATACCAGACTATAACACATATAAAAATACGTGTGATGG |
| | TACAACATTTACTTATGCATCAGCATTGTGGGAAATCCAACAGGTTGTAGATGCAGATAGTAAAATT |
| | GTTCAACTTAGTGAAATTAGTATGGACAATTCACCTAATTTAGCATGGCCTCTTATTGTAACAGCTT |
| | TAAGGGCCAATTCTGCTGTCAAATTACAGAATAATGAGCTTAGTCCTGTTGCACTACGACAGATGTC |
| | TTGTGCTGCCGGTACTACACAAACTGCTTGCACTGATGACAATGCGTTAGCTTACTACAACACAACA |
| | AAGGGAGGTAGGTTTGTACTTGCACTGTTATCCGATTTACAGGATTTGAAATGGGCTAGATTCCCTA |
| | AGAGTGATGGAACTGGTACTATCTATACAGAACTGGAACCACCTTGTAGGTTTGTTACAGACACACC |
| | TAAAGGTCCTAAAGTGAAGTATTTATACTTTATTAAAGGATTAAACAACCTAAATAGAGGTATGGTA |
| | CTTGGTAGTTTAGCTGCCACAGTACGTCTACAAGCTGGTAATGCAACAGAAGTGCCTGCCAATTCAA |
| | CTGTATTATCTTTCTGTGCTTTTGCTGTAGATGCTGCTAAAGCTTACAAAGATTATCTAGCTAGTGG |
| | GGGACAACCAATCACTAATTGTGTTAAGATGTTGTGTACACACACTGGTACTGGTCAGGCAATAACA |
| | GTTACACCGGAAGCCAATATGGATCAAGAATCCTTTGGTGGTGCATCGTGTTGTCTGTACTGCCGTT |
| | GCCACATAGATCATCCAAATCCTAAAGGATTTTGTGACTTAAAAGGTAAGTATGTACAAATACCTAC |
| | AACTTGTGCTAATGACCCTGTGGGTTTTACACTTAAAAACACAGTCTGTACCGTCTGCGGTATGTGG |
| | AAAGGTTATGGCTGTAGTTGTGATCAACTCCGCGAACCCATGCTTCAGTCAGCTGATGCACAATCGT |
| | TTTTAAACGGGTTTGCGGTGTAAGTGCAGCCCGTCTTACACCGTGCGGCACAGGCACTAGTACTGAT |
| | GTCGTATACAGGGCTTTTGACATCTACAATGATAAAGTAGCTGGTTTTGCTAAATTCCTAAAAACTA |
| | ATTGTTGTCGCTTCCAAGAAAAGGACGAAGATGACAATTTAATTGATTCTTACTTTGTAGTTAAGAG |
| | ACACACTTTCTCTAACTACCAACATGAAGAAACAATTTATAATTTACTTAAGGATTGTCCAGCTGTT |
| | GCTAAACATGACTTCTTTAAGTTTAGAATAGACGGTGACATGGTACCACATATATCACGTCAACGTC |
| | TTACTAAATACACAATGGCAGACCTCGTCTATGCTTTAAGGCATTTTGATGAAGGTAATTGTGACAC |
| | ATTAAAAGAAATACTTGTCACATACAATTGTTGTGATGATGATTATTTCAATAAAAAGGACTGGTAT |
| | GATTTTGTAGAAAACCCAGATATATTACGCGTATACGCCAACTTAGGTGAACGTGTACGCCAAGCTT |
| | TGTTAAAAACAGTACAATTCTGTGATGCCATGCGAAATGCTGGTATTGTTGGTGTACTGACATTAGA |
| | TAATCAAGATCTCAATGGTAACTGGTATGATTTCGGTGATTTCATACAAACCACGCCAGGTAGTGGA |
| | GTTCCTGTTGTAGATTCTTATTATTCATTGTTAATGCCTATATTAACCTTGACCAGGGCTTTAACTG |
| | CAGAGTCACATGTTGACACTGACTTAACAAAGCCTTACATTAAGTGGGATTTGTTAAAATATGACTT |
| | CACGGAAGAGAGGTTAAAACTCTTTGACCGTTATTTTAAATATTGGGATCAGACATACCACCCAAAT |
| | TGTGTTAACTGTTTGGATGACAGATGCATTCTGCATTGTGCAAACTTTAATGTTTTATTCTCTACAG |
| | TGTTCCCACTTACAAGTTTTGGACCACTAGTGAGAAAAATATTTGTTGATGGTGTTCCATTTGTAGT |
| | TTCAACTGGATACCACTTCAGAGAGCTAGGTGTTGTACATAATCAGGATGTAAACTTACATAGCTCT |
| | AGACTTAGTTTTAAGGAATTACTTGTGTATGCTGCTGACCCTGCTATGCACGCTGCTTCTGGTAATC |
| | TATTACTAGATAAACGCACTACGTGCTTTTCAGTAGCTGCACTTACTAACAATGTTGCTTTTCAAAC |
| | TGTCAAACCTGGTAATTTTAACAAAGACTTCTATGACTTTGCTGTGTCTAAGGGTTTCTTTAAGGAA |
| | GGAAGTTCTGTTGAATTAAAACACTTCTTCTTTGCTCAGGATGGTAATGCTGCTATCAGCGATTATG |
| | ACTACTATCGTTATAATCTACCAACAATGTGTGATATCAGACAACTACTATTTGTAGTTGAAGTTGT |
| | TGATAAGTACTTTGATTGTTACGATGGTGGCTGTATTAATGCTAACCAAGTCATCGTCAACAACCTA |
| | GACAAATCAGCTGGTTTTCCATTTAATAAATGGGGTAAGGCTAGACTTTATTATGATTCAATGAGTT |
| | ATGAGGATCAAGATGCACTTTTCGCATATACAAAACGTAATGTCATCCCTACTATAACTCAAATGAA |
| | TCTTAAGTATGCCATTAGTGCAAAGAATAGAGCTCGCACCGTAGCTGGTGTCTCTATCTGTAGTACT |
| | ATGACCAATAGACAGTTTCATCAAAAATTATTGAAATCAATAGCCGCCACTAGAGGAGCTACTGTAG |
| | TAATTGGAACAAGCAAATTCTATGGTGGTTGGCACAACATGTTAAAAACTGTTTATAGTGATGTAGA |
| | AAACCCTCATCTTATGGGTTGGGATTATCCTAAATGTGATAGAGCCATGCCTAACATGCTTAGAATT |
| | ATGGCCTCACTTGTTCTTGCTCGCAAACATACAACGTGTTGTAGCTTGTCACACCGTTTCTATAGAT |
| | TAGCTAATGAGTGTGCTCAAGTATTGAGTGAAATGGTCATGTGTGGCGGTTCACTATATGTTAAACC |
| | AGGTGGAACCTCATCAGGAGATGCCACAACTGCTTATGCTAATAGTGTTTTTAACATTTGTCAAGCT |
| | GTCACGGCCAATGTTAATGCACTTTTATCTACTGATGGTAACAAAATTGCCGATAAGTATGTCCGCA |
| | ATTTACAACACAGACTTTATGAGTGTCTCTATAGAAATAGAGATGTTGACACAGACTTTGTGAATGA |
| | GTTTTACGCATATTTGCGTAAACATTTCTCAATGATGATACTCTCTGACGATGCTGTTGTGTGTTTC |
| | AATAGCACTTATGCATCTCAAGGTCTAGTGGCTAGCATAAAGAACTTTAAGTCAGTTCTTTATTATC |
| | AAAACAATGTTTTTATGTCTGAAGCAAAATGTTGGACTGAGACTGACCTTACTAAAGGACCTCATGA |
| | ATTTTGCTCTCAACATACAATGCTAGTTAAACAGGGTGATGATTATGTGTACCTTCCTTACCCAGAT |
| | CCATCAAGAATCCTAGGGGCCGGCTGTTTTGTAGATGATATCGTAAAAACAGATGGTACACTTATGA |
| | TTGAACGGTTCGTGTCTTTAGCTATAGATGCTTACCCACTTACTAAACATCCTAATCAGGAGTATGC |
| | TGATGTCTTTCATTTGTACTTACAATACATAAGAAAGCTACATGATGAGTTAACAGGACACATGTTA |
| | GACATGTATTCTGTTATGCTTACTAATGATAACACCTCAAGGTATTGGGAACCTGAGTTTTATGAGG |
| | CTATGTACACACCGCATACAGTCTTACAGGCTGTTGGGGCTTGTGTTCTTTGCAATTCACAGACTTC |
| | ATTAAGATGTGGTGCTTGCATACGTAGACCATTCTTATGTTGTAAATGCTGTTACGACCATGTCATA |
| | TCAACATCACATAAATTAGTCTTGTCTGTTAATCCGTATGTTTGCAATGCTCCAGGTTGTGATGTCA |
| | CAGATGTGACTCAACTTTACTTAGGAGGTATGAGCTATTATTGTAAATCACATAAACCATCCATTAG |
| | TTTTCCATTGTGTGCTAATGGACAAGTTTTTGGTTTATATAAAAATACATGTGTTGGTAGCGATAAT |
| | GTTACTGACTTTAATGCAATTGCAACATGTGACTGGACAAATGCTGGTGATTACATTTTAGCTAACA |
| | CCTGTACTGAAAGACTCAAGCTTTTTGCAGCAGAAACGCTCAAAGCTACTGAGGAGACATTTAAACT |
| | GTCTTATGGTATTGCTACTGTACGTGAAGTGCTGTCTGACAGAGAATTACATCTTTCATGGGAAGTT |
| | GGTAAACCTAGACCACCACTTAACCGAAATTATGTCTTTACTGGTTATCGTGTAACTAAAAACAGTA |
| | AAGTACAAATAGGAGAGTACACCTTTGAAAAAGGTGACTATGGTGATGCTGTTGTTTACCGAGGTAC |
| | AACAACTTACAAATTAAATGTTGGTGATTATTTTGTGCTGACATCACATACAGTAATGCCATTAAGT |
| | GCACCTACACTAGTGCCACAAGAGCACTATGTTAGAATTACTGGCTTATACCCAACACTCAATATCT |
| | CAGATGAGTTTTCTAGCAATGTTGCAAATTATCAAAAGGTTGGTATGCAAAAGTATTCTACACTCCA |
| | GGGACCACCTGGTACTGGTAAGAGTCATTTTGCTATTGGCCTAGCTCTCTACTACCCTTCTGCTCGC |
| | ATAGTGTATACAGCTTGCTCTCATGCCGCTGTTGATGCACTATGTGAGAAGGCATTAAAATATTTGC |
| | CTATAGATAAATGTAGTAGAATTATACCTGCACGTGCTCGTGTAGAGTGTTTTGATAAATTCAAAGT |
| | GAATTCAACATTAGAACAGTATGTCTTTTGTACTGTAAATGCATTGCCTGAGACGACAGCAGATATA |
| | GTTGTCTTTGATGAAATTTCAATGGCCACAAATTATGATTTGAGTGTTGTCAATGCCAGATTACGTG |
| | CTAAGCACTATGTGTACATTGGCGACCCTGCTCAATTACCTGCACCACGCACATTGCTAACTAAGGG |
| | CACACTAGAACCAGAATATTTCAATTCAGTGTGTAGACTTATGAAAACTATAGGTCCAGACATGTTC |
| | CTCGGAACTTGTCGGCGTTGTCCTGCTGAAATTGTTGACACTGTGAGTGCTTTGGTTTATGATAATA |
| | GGCTTAAAGCACATAAAGACAAATCAGCTCAATGCTTTAAAATGTTTTATAAGGGTGTTATCACGCA |
| | TGATGTTTCATCTGCAATTAACAGGCCACAAATAGGCGTGGTAAGAGAATTCCTTACACGTAACCCT |
| | GCTTGGAGAAAAGCTGTCTTTATTTCACCTTATAATTCACAGAATGCTGTAGCCTCAAAGATTTTGG |
| | GACTACCAACTCAAACTGTTGATTCATCACAGGGCTCAGAATATGACTATGTCATATTCACTCAAAC |
| | CACTGAAACAGCTCACTCTTGTAATGTAAACAGATTTAATGTTGCTATTACCAGAGCAAAAGTAGGC |
| | ATACTTTGCATAATGTCTGATAGAGACCTTTATGACAAGTTGCAATTTACAAGTCTTGAAATTCCAC |
| | GTAGGAATGTGGCAACTTTACAAGCTGAAAATGTAACAGGACTCTTTAAAGATTGTAGTAAGGTAAT |
| | CACTGGGTTACATCCTACACAGGCACCTACACACCTCAGTGTTGACACTAAATTCAAAACTGAAGGT |
| | TTATGTGTTGACATACCTGGCATACCTAAGGACATGACCTATAGAAGACTCATCTCTATGATGGGTT |
| | TTAAAATGAATTATCAAGTTAATGGTTACCCTAACATGTTTATCACCCGCGAAGAAGCTATAAGACA |
| | TGTACGTGCATGGATTGGCTTCGATGTCGAGGGGTGTCATGCTACTAGAGAAGCTGTTGGTACCAAT |
| | TTACCTTTACAGCTAGGTTTTTCTACAGGTGTTAACCTAGTTGCTGTACCTACAGGTTATGTTGATA |
| | CACCTAATAATACAGATTTTTCCAGAGTTAGTGCTAAACCACCGCCTGGAGATCAATTTAAACACCT |
| | CATACCACTTATGTACAAAGGACTTCCTTGGAATGTAGTGCGTATAAAGATTGTACAAATGTTAAGT |
| | GACACACTTAAAAATCTCTCTGACAGAGTCGTATTTGTCTTATGGGCACATGGCTTTGAGTTGACAT |
| | CTATGAAGTATTTTGTGAAAATAGGACCTGAGCGCACCTGTTGTCTATGTGATAGACGTGCCACATG |
| | CTTTTCCACTGCTTCAGACACTTATGCCTGTTGGCATCATTCTATTGGATTTGATTACGTCTATAAT |
| | CCGTTTATGATTGATGTTCAACAATGGGGTTTTACAGGTAACCTACAAAGCAACCATGATCTGTATT |
| | GTCAAGTCCATGGTAATGCACATGTAGCTAGTTGTGATGCAATCATGACTAGGTGTCTAGCTGTCCA |
| | CGAGTGCTTTGTTAAGCGTGTTGACTGGACTATTGAATATCCTATAATTGGTGATGAACTGAAGATT |
| | AATGCGGCTTGTAGAAAGGTTCAACACATGGTTGTTAAAGCTGCATTATTAGCAGACAAATTCCCAG |
| | TTCTTCACGACATTGGTAACCCTAAAGCTATTAAGTGTGTACCTCAAGCTGATGTAGAATGGAAGTT |
| | CTATGATGCACAGCCTTGTAGTGACAAAGCTTATAAAATAGAAGAATTATTCTATTCTTATGCCACA |
| | CATTCTGACAAATTCACAGATGGTGTATGCCTATTTTGGAATTGCAATGTCGATAGATATCCTGCTA |
| | ATTCCATTGTTTGTAGATTTGACACTAGAGTGCTATCTAACCTTAACTTGCCTGGTTGTGATGGTGG |
| | CAGTTTGTATGTAAATAAACATGCATTCCACACACCAGCTTTTGATAAAAGTGCTTTTGTTAATTTA |
| | AAACAATTACCATTTTTCTATTACTCTGACAGTCCATGTGAGTCTCATGGAAAACAAGTAGTGTCAG |
| | ATATAGATTATGTACCACTAAAGTCTGCTACGTGTATAACACGTTGCAATTTAGGTGGTGCTGTCTG |
| | TAGACATCATGCTAATGAGTACAGATTGTATCTCGATGCTTATAACATGATGATCTCAGCTGGCTTT |
| | AGCTTGTGGGTTTACAAACAATTTGATACTTATAACCTCTGGAACACTTTTACAAGACTTCAGAGTT |
| | TAGAAAATGTGGCTTTTAATGTTGTAAATAAGGGACACTTTGATGGACAACAGGGTGAAGTACCAGT |
| | TTCTATCATTAATAACACTGTTTACACAAAAGTTGATGGTGTTGATGTAGAATTGTTTGAAAATAAA |
| | ACAACATTACCTGTTAATGTAGCATTTGAGCTTTGGGCTAAGCGCAACATTAAACCAGTACCAGAGG |
| | TGAAAATACTCAATAATTTGGGTGTGGACATTGCTGCTAATACTGTGATCTGGGACTACAAAAGAGA |
| | TGCTCCAGCACATATATCTACTATTGGTGTTTGTTCTATGACTGACATAGCCAAGAAACCAACTGAA |
| | ACGATTTGTGCACCACTCACTGTCTTTTTTGATGGTAGAGTTGATGGTCAAGTAGACTTATTTAGAA |
| | ATGCCCGTAATGGTGTTCTTATTACAGAAGGTAGTGTTAAAGGTTTACAACCATCTGTAGGTCCCAA |
| | ACAAGCTAGTCTTAATGGAGTCACATTAATTGGAGAAGCCGTAAAAACACAGTTCAATTATTATAAG |
| | AAAGTTGATGGTGTTGTCCAACAATTACCTGAAACTTACTTTACTCAGAGTAGAAATTTACAAGAAT |
| | TTAAACCCAGGAGTCAAATGGAAATTGATTTCTTAGAATTAGCTATGGATGAATTCATTGAACGGTA |
| | TAAATTAGAAGGCTATGCCTTCGAACATATCGTTTATGGAGATTTTAGTCATAGTCAGTTAGGTGGT |
| | TTACATCTACTGATTGGACTAGCTAAACGTTTTAAGGAATCACCTTTTGAATTAGAAGATTTTATTC |
| | CTATGGACAGTACAGTTAAAAACTATTTCATAACAGATGCGCAAACAGGTTCATCTAAGTGTGTGTG |
| | TTCTGTTATTGATTTATTACTTGATGATTTTGTTGAAATAATAAAATCCCAAGATTTATCTGTAGTT |
| | TCTAAGGTTGTCAAAGTGACTATTGACTATACAGAAATTTCATTTATGCTTTGGTGTAAAGATGGCC |
| | ATGTAGAAACATTTTACCCAAAATTACAATCTAGTCAAGCGTGGCAACCGGGTGTTGCTATGCCTAA |
| | TCTTTACAAAATGCAAAGAATGCTATTAGAAAAGTGTGACCTTCAAAATTATGGTGATAGTGCAACA |
| | TTACCTAAAGGCATAATGATGAATGTCGCAAAATATACTCAACTGTGTCAATATTTAAACACATTAA |
| | CATTAGCTGTACCCTATAATATGAGAGTTATACATTTTGGTGCTGGTTCTGATAAAGGAGTTGCACC |
| | AGGTACAGCTGTTTTAAGACAGTGGTTGCCTACGGGTACGCTGCTTGTCGATTCAGATCTTAATGAC |
| | TTTGTCTCTGATGCAGATTCAACTTTGATTGGTGATTGTGCAACTGTACATACAGCTAATAAATGGG |
| | ATCTCATTATTAGTGATATGTACGACCCTAAGACTAAAAATGTTACAAAAGAAAATGACTCTAAAGA |
| | GGGTTTTTTCACTTACATTTGTGGGTTTATACAACAAAAGCTAGCTCTTGGAGGTTCCGTGGCTATA |
| | AAGATAACAGAACATTCTTGGAATGCTGATCTTTATAAGCTCATGGGACACTTCGCATGGTGGACAG |
| | CCTTTGTTACTAATGTGAATGCGTCATCATCTGAAGCATTTTTAATTGGATGTAATTATCTTGGCAA |
| | ACCACGCGAACAAATAGATGGTTATGTCATGCATGCAAATTACATATTTTGGAGGAATACAAATCCA |
| | ATTCAGTTGTCTTCCTATTCTTTATTTGACATGAGTAAATTTCCCCTTAAATTAAGGGGTACTGCTG |
| | TTATGTCTTTAAAAGAAGGTCAAATCAATGATATGATTTTATCTCTTCTTAGTAAAGGTAGACTTAT |
| | AATTAGAGAAAACAACAGAGTTGTTATTTCTAGTGATGTTCTTGTTAACAACTAAACGAACAATGTT |
| | TGTTTTTCTTGTTTTATTGCCACTAGTCTCTAGTCAGTGTGTTAATCTTACAACCAGAACTCAATTA |
| | CCCCCTGCATACACTAATTCTTTCACACGTGGTGTTTATTACCCTGACAAAGTTTTCAGATCCTCAG |
| | TTTTACATTCAACTCAGGACTTGTTCTTACCTTTCTTTTCCAATGTTACTTGGTTCCATGCTATCTC |
| | TGGGACCAATGGTACTAAGAGGTTTGATAACCCTGTCCTACCATTTAATGATGGTGTTTATTTTGCT |
| | TCCACTGAGAAGTCTAACATAATAAGAGGCTGGATTTTTGGTACTACTTTAGATTCGAAGACCCAGT |
| | CCCTACTTATTGTTAATAACGCTACTAATGTTGTTATTAAAGTCTGTGAATTTCAATTTTGTAATGA |
| | TCCATTTTTGGGTGTTTACCACAAAAACAACAAAAGTTGGATGGAAAGTGAGTTCAGAGTTTATTCT |
| | AGTGCGAATAATTGCACTTTTGAATATGTCTCTCAGCCTTTTCTTATGGACCTTGAAGGAAAACAGG |
| | GTAATTTCAAAAATCTTAGGGAATTTGTGTTTAAGAATATTGATGGTTATTTTAAAATATATTCTAA |
| | GCACACGCCTATTAATTTAGTGCGTGATCTCCCTCAGGGTTTTTCGGCTTTAGAACCATTGGTAGAT |
| | TTGCCAATAGGTATTAACATCACTAGGTTTCAAACTTTACTTGCTTTACATAGAAGTTATTTGACTC |
| | CTGGTGATTCTTCTTCAGGTTGGACAGCTGGTGCTGCAGCTTATTATGTGGGTTATCTTCAACCTAG |
| | GACTTTTCTATTAAAATATAATGAAAATGGAACCATTACAGATGCTGTAGACTGTGCACTTGACCCT |
| | CTCTCAGAAACAAAGTGTACGTTGAAATCCTTCACTGTAGAAAAAGGAATCTATCAAACTTCTAACT |
| | TTAGAGTCCAACCAACAGAATCTATTGTTAGATTTCCTAATATTACAAACTTGTGCCCTTTTGGTGA |
| | AGTTTTTAACGCCACCAGATTTGCATCTGTTTATGCTTGGAACAGGAAGAGAATCAGCAACTGTGTT |
| | GCTGATTATTCTGTCCTATATAATTCCGCATCATTTTCCACTTTTAAGTGTTATGGAGTGTCTCCTA |
| | CTAAATTAAATGATCTCTGCTTTACTAATGTCTATGCAGATTCATTTGTAATTAGAGGTGATGAAGT |
| | CAGACAAATCGCTCCAGGGCAAACTGGAAAGATTGCTGATTATAATTATAAATTACCAGATGATTTT |
| | ACAGGCTGCGTTATAGCTTGGAATTCTAACAATCTTGATTCTAAGGTTGGTGGTAATTATAATTACC |
| | TGTATAGATTGTTTAGGAAGTCTAATCTCAAACCTTTTGAGAGAGATATTTCAACTGAAATCTATCA |
| | GGCCGGTAGCACACCTTGTAATGGTGTTGAAGGTTTTAATTGTTACTTTCCTTTACAATCATATGGT |
| | TTCCAACCCACTTATGGTGTTGGTTACCAACCATACAGAGTAGTAGTACTTTCTTTTGAACTTCTAC |
| | ATGCACCAGCAACTGTTTGTGGACCTAAAAAGTCTACTAATTTGGTTAAAAACAAATGTGTCAATTT |
| | CAACTTCAATGGTTTAACAGGCACAGGTGTTCTTACTGAGTCTAACAAAAAGTTTCTGCCTTTCCAA |
| | CAATTTGGCAGAGACATTGATGACACTACTGATGCTGTCCGTGATCCACAGACACTTGAGATTCTTG |
| | ACATTACACCATGTTCTTTTGGTGGTGTCAGTGTTATAACACCAGGAACAAATACTTCTAACCAGGT |
| | TGCTGTTCTTTATCAGGGTGTTAACTGCACAGAAGTCCCTGTTGCTATTCATGCAGATCAACTTACT |
| | CCTACTTGGCGTGTTTATTCTACAGGTTCTAATGTTTTTCAAACACGTGCAGGCTGTTTAATAGGGG |
| | CTGAACATGTCAACAACTCATATGAGTGTGACATACCCATTGGTGCAGGTATATGCGCTAGTTATCA |
| | GACTCAGACTAATTCTCATCGGCGGGCACGTAGTGTAGCTAGTCAATCCATCATTGCCTACACTATG |
| | TCACTTGGTGCAGAAAATTCAGTTGCTTACTCTAATAACTCTATTGCCATACCCATAAATTTTACTA |
| | TTAGTGTTACCACAGAAATTCTACCAGTGTCTATGACCAAGACATCAGTAGATTGTACAATGTACAT |
| | TTGTGGTGATTCAACTGAATGCAGCAATCTTTTGTTGCAATATGGCAGTTTTTGTACACAATTAAAC |
| | CGTGCTTTAACTGGAATAGCTGTTGAACAAGACAAAAACACCCAAGAAGTTTTTGCACAAGTCAAAC |
| | AAATTTACAAAACACCACCAATTAAAGATTTTGGTGGTTTTAATTTTTCACAAATATTACCAGATCC |
| | ATCAAAACCAAGCAAGAGGTCATTTATTGAAGATCTACTTTTCAACAAAGTGACACTTGCAGATGCT |
| | GGCTTCATCAAACAATATGGTGATTGCCTTGGTGATATTGCTGCTAGAGACCTCATTTGTGCACAAA |
| | AGTTTAACGGCCTTACTGTTTTGCCACCTTTGCTCACAGATGAAATGATTGCTCAATACACTTCTGC |
| | ACTGTTAGCGGGTACAATCACTTCTGGTTGGACCTTTGGTGCAGGTGCTGCATTACAAATACCATTT |
| | GCTATGCAAATGGCTTATAGGTTTAATGGTATTGGAGTTACACAGAATGTTCTCTATGAGAACCAAA |
| | AATTGATTGCCAACCAATTTAATAGTGCTATTGGCAAAATTCAAGACTCACTTTCTTCCACAGCAAG |
| | TGCACTTGGAAAACTTCAAGATGTGGTCAACCAAAATGCACAAGCTTTAAACACGCTTGTTAAACAA |
| | CTTAGCTCCAATTTTGGTGCAATTTCAAGTGTTTTAAATGATATCCTTGCACGTCTTGACAAAGTTG |
| | AGGCTGAAGTGCAAATTGATAGGTTGATCACAGGCAGACTTCAAAGTTTGCAGACATATGTGACTCA |
| | ACAATTAATTAGAGCTGCAGAAATCAGAGGTTGTGCTAATCTTGOTGCTACTAAAATGTCAGAGTGT |
| | GTACTTGGACAATCAAAAAGAGTTGATTTTTGTGGAAAGGGCTATCATCTTATGTCCTTCCCTCAGT |
| | CAGCACCTCATGGTGTAGTCTTCTTGCATGTGACTTATGTCCCTGCACAAGAAAAGAACTTCACAAC |
| | TGCTCCTGCCATTTGTCATGATGGAAAAGCACACTTTCCTCGTGAAGGTGTCTTTGTTTCAAATGGC |
| | ACACACTGGTTTGTAACACAAAGGAATTTTTATGAACCACAAATCATTACTACACACAACACATTTG |
| | TGTCTGGTAACTGTGATGTTGTAATAGGAATTGTCAACAACACAGTTTATGATCCTTTGCAACCTGA |
| | ATTAGACTCATTCAAGGAGGAGTTAGATAAATATTTTAAGAATCATACATCACCAGATGTTGATTTA |
| | GGTGACATCTCTGGCATTAATGCTTCAGTTGTAAACATTCAAAAAGAAATTGACCGCCTCAATGAGG |
| | TTGCCAAGAATTTAAATGAATCTCTCATCGATCTCCAAGAACTTGGAAAGTATGAGCAGTATATAAA |
| | ATGGCCATGGTACATTTGGCTAGGTTTTATAGCTGGCTTGATTGCCATAGTAATGGTGACAATTATG |
| | CTTTGCTGTATGACCAGTTGCTGTAGTTGTCTCAAGGGCTGTTGTTCTTGTGGATCCTGCTGCAAAT |
| | TTGATGAAGACGACTCTGAGCCAGTGCTCAAAGGAGTCAAATTACATTACACATAAACGAACTTATG |
| | GATTTGTTTATGAGAATCTTCACAATTGGAACTGTAACTTTGAAGCAAGGTGAAATCAAGGATGCTA |
| | CTCCTTCAGATTTTGTTCGCGCTACTGCAACGATACCGATACAAGCCTCACTCCCTTTCGGATGGCT |
| | TATTGTTGGCGTTGCACTTCTTGCTGTTTTTCAGAGCGCTTCCAAAATCATAACCCTCAAAAAGAGA |
| | TGGCAACTAGCACTCTCCAAGGGTGTTCACTTTGTTTGCAACTTGCTGTTGTTGTTTGTAACAGTTT |
| | ACTCACACCTTTTGCTCGTTGCTGCTGGCCTTGAAGCCCCTTTTCTCTATCTTTATGCTTTAGTCTA |
| | CTTCTTGCAGAGTATAAACTTTGTAAGAATAATAATGAGGCTTTGGCTTTGCTGGAAATGCCGTTCC |
| | AAAAACCCATTACTTTATGATGCCAACTATTTTCTTTGCTGGCATACTAATTGTTACGACTATTGTA |
| | TACCTTACAATAGTGTAACTTCTTCAATTGTCATTACTTCAGGTGATGGCACAACAAGTCCTATTTC |
| | TGAACATGACTACCAGATTGGTGGTTATACTGAAAAATGGGAATCTGGAGTAAAAGACTGTGTTGTA |
| | TTACACAGTTACTTCACTTCAGACTATTACCAGCTGTACTCAACTCAATTGAGTACAGACACTGGTG |
| | TTGAACATGTTACCTTCTTCATCTACAATAAAATTGTTGATGAGCCTGAAGAACATGTCCAAATTCA |
| | CACAATCGACGGTTCATCCGGAGTTGTTAATCCAGTAATGGAACCAATTTATGATGAACCGACGACG |
| | ACTACTAGCGTGCCTTTGTAAGCACAAGCTGATGAGTACGAACTTATGTACTCATTCGTTTCGGAAG |
| | AGACAGGTACGTTAATAGTTAATAGCGTACTTCTTTTTCTTGCTTTCGTGGTATTCTTGCTAGTTAC |
| | ACTAGCCATCCTTACTGCGCTTCGATTGTGTGCGTACTGCTGCAATATTGTTAACGTGAGTCTTGTA |
| | AAACCTTCTTTTTACGTTTACTCTCGTGTTAAAAATCTGAATTCTTCTAGAGTTCCTGATCTTCTGG |
| | TCTAAACGAACTAAATATTATATTAGTTTTTCTGTTTGGAACTTTAATTTTAGCCATGGCAGATTCC |
| | AACGGTACTATTACCGTTGAAGAGCTTAAAAAGCTCCTTGAACAATGGAACCTAGTAATAGGTTTCC |
| | TATTCCTTACATGGATTTGTCTTCTACAATTTGCCTATGCCAACAGGAATAGGTTTTTGTATATAAT |
| | TAAGTTAATTTTCCTCTGGCTGTTATGGCCAGTAACTTTAGCTTGTTTTGTGCTTGCTGCTGTTTAC |
| | AGAATAAATTGGATCACCGGTGGAATTGCTATCGCAATGGCTTGTCTTGTAGGCTTGATGTGGCTCA |
| | GCTACTTCATTGCTTCTTTCAGACTGTTTGCGCGTACGCGTTCCATGTGGTCATTCAATCCAGAAAC |
| | TAACATTCTTCTCAACGTGCCACTCCATGGCACTATTCTGACCAGACCGCTTCTAGAAAGTGAACTC |
| | GTAATCGGAGCTGTGATCCTTCGTGGACATCTTCGTATTGCTGGACACCATCTAGGACGCTGTGACA |
| | TCAAGGACCTGCCTAAAGAAATCACTGTTGCTACATCACGAACGCTTTCTTATTACAAATTGGGAGC |
| | TTCGCAGCGTGTAGCAGGTGACTCAGGTTTTGCTGCATACAGTCGCTACAGGATTGGCAACTATAAA |
| | TTAAACACAGACCATTCCAGTAGCAGTGACAATATTGCTTTGCTTGTACAGTAAGTGACAACAGATG |
| | TTTCATCTCGTTGACTTTCAGGTTACTATAGCAGAGATATTACTAATTATTATGAGGACTTTTAAAG |
| | TTTCCATTTGGAATCTTGATTACATCATAAACCTCATAATTAAAAATTTATCTAAGTCACTAACTGA |
| | GAATAAATATTCTCAATTAGATGAAGAGCAACCAATGGAGATTGATTAAACGAACATGAAAATTATT |
| | CTTTTCTTGGCACTGATAACACTCGCTACTTGTGAGCTTTATCACTACCAAGAGTGTGTTAGAGGTA |
| | CAACAGTACTTTTAAAAGAACCTTGCTCTTCTGGAACATACGAGGGCAATTCACCATTTCATCCTCT |
| | AGCTGATAACAAATTTGCACTGACTTGCTTTAGCACTCAATTTGCTTTTGCTTGTCCTGACGGCGTA |
| | AAACACGTCTATCAGTTACGTGCCAGATCAGTTTCACCTAAACTGTTCATCAGACAAGAGGAAGTTC |
| | AAGAACTTTACTCTCCAATTTTTCTTATTGTTGCGGCAATAGTGTTTATAACACTTTGCTTCACACT |
| | CAAAAGAAAGACAGAATGATTGAACTTTCATTAATTGACTTCTATTTGTGCTTTTTAGCCTTTCTGC |
| | TATTCCTTGTTTTAATTATGCTTATTATCTTTTGGTTCTCACTTGAACTGCAAGATCATAATGAAAC |
| | TTGTCACGCCTAAACGAACATGAAATTTCTTGTTTTCTTAGGAATCATCACAACTGTAGCTGCATTT |
| | CACCAAGAATGTAGTTTACAGTCATGTACTTAACATCAACCATATGTAGTTGATGACCCGTGTCCTA |
| | TTCACTTCTATTCTAAATGGTATATTAGAGTAGGAGCTATAAAATCAGCACCTTTAATTGAATTGTG |
| | CGTGGATGAGGCTGGTTCTAAATCACCCATTCAGTGCATCGATATCGGTAATTATACAGTTTCCTGT |
| | TTACCTTTTACAATTAATTGCCAGGAACCTAAATTGGGTAGTCTTGTAGTGCGTTGTTCGTTCTATG |
| | AAGACTTTTTAGAGTATCATGACGTTCGTGTTGTTTTAGATTTCATCTAAACGAACAAACTAAATGT |
| | CTCTAAATGGACCCCAAAATCAGCGAAATGCACCCCGCATTACGTTTGGTGGACCCTCAGATTCAAC |
| | TGGCAGTAACCAGAATGGAGAACGCAGTGGGGCGCGATCAAAACAACGTCGGCCCCAAGGTTTACCC |
| | AATAATACTGCGTCTTGGTTCACCGCTCTCACTCAACATGGCAAGGAAGACCTTAAATTCCCTCGAG |
| | GACAAGGCGTTCCAATTAACACCAATAGCAGTCCAGATGACCAAATTGGCTACTACCGAAGAGCTAC |
| | CAGACGAATTCGTGGTGGTGACGGTAAAATGAAAGATCTCAGTCCAAGATGGTATTTCTACTACCTA |
| | GGAACTGGGCCAGAAGCTGGACTTCCCTATGGTGCTAACAAAGACGGCATCATATGGGTTGCAACTG |
| | AGGGAGCCTTGAATACACCAAAAGATCACATTGGCACCCGCAATCCTGCTAACAATGCTGCAATCGT |
| | GCTACAACTTCCTCAAGGAACAACATTGCCAAAAGGCTTCTACGCAGAAGGGAGCAGAGGCGGCAGT |
| | CAAGCCTCTTCTCGTTCCTCATCACGTAGTCGCAACAGTTCAAGAAATTCAACTCCAGGCAGCAGTA |
| | AACGAACTTCTCCTGCTAGAATGGCTGGCAATGGCGGTGATGCGGCTCTTGCTTTGCTGCTGCTTGA |
| | CAGATTGAACCAGCTTGAGAGCAAAATGTTTGGTAAAGGCCAACAACAACAAGGCCAAACTGTCACT |
| | AAGAAATCTGCTGCTGAGGCTTCTAAGAAGCCTCGGCAAAAACGTACTGCCACTAAAGCATACAATG |
| | TAACACAAGCTTTCGGCAGACGTGGTCCAGAACAAACCCAAGGAAATTTTGGGGACCAGGAACTAAT |
| | CAGACAAGGAACTGATTACAAACATTGGCCGCAAATTGCACAATTTGCCCCCAGCGCTTCAGCGTTC |
| | TTCGGAATGTCGCGCATTGGCATGGAAGTCACACCTTCGGGAACGTGGTTGACCTACACAGGTGCCA |
| | TCAAATTGGATGACAAAGATCCAAATTTCAAAGATCAAGTCATTTTGCTGAATAAGCATATTGACGC |
| | ATACAAAACATTCCCACCAACAGAGCCTAAAAAGGACAAAAAGAAGAAGGCTGATGAAACTCAAGCC |
| | TTACCGCAGAGACAGAAGAAACAGCAAACTGTGACTCTTCTTCCTGCTGCAGATTTGGATGATTTCT |
| | CCAAACAATTGCAACAATCCATGAGCAGTGCTGACTCAACTCAGGCCTAAACTCATGCAGACCACAC |
| | AAGGCAGATGGGCTATATAAACGTTTTCGCTTTTCCGTTTACGATATATAGTCTACTCTTGTGCAGA |
| | ATGAATTCTCGTAACTACATAGCACAAGTAGATGTAGTTAACTTTAATCTCACATAGCAATCTTTAA |
| | TCAGTGTGTAACATTAGGGAGGACTTGAAAGAGCCACCACATTTTCACCGAGGCCACGCGGAGTACG |
| | ATCGAGTGTACAGTGAACAATGCTAGGGAGAGCTGCCTATATGGAAGAGCCCTAATGTGTAAAATTA |
| | ATTTTAGTAGTGCTATCCCCATGTGATTTTAATAGCTTCTTAGGAGAA |
| 293 | GenBank Accession No. MW642250 (P.1 SARS-CoV-2 variant; Severe acute |
| | respiratory syndrome coronavirus 2 isolate SARS-CoV-2/human/ITA/ABR- |
| | IZSGC-TE30968/2021, complete genome) |
| | ATTAAAGGTTTATACCTTCCCAGGTAACAAACCAACCAACTTTCGATCTCTTGTAGATCTGTTCTCT |
| | AAACGAACTTTAAAATCTGTGTGGCTGTCACTCGGCTGCATGCTTAGTGCACTCACGCAGTATAATT |
| | AATAACTAATTACTGTCGTTGACAGGACACGAGTAACTCGTCTATCTTCTGCAGGCTGCTTACGGTT |
| | TCGTCCGTGTTGCAGCCGATCATCAGCACATCTAGGTTTTGTCCGGGTGTGACCGAAAGGTAAGATG |
| | GAGAGCCTTGTCCCTGGTTTCAACGAGAAAACACACGTCCAACTCAGTTTGCCTGTTTTACAGGTTC |
| | GCGACGTGCTCGTACGTGGCTTTGGAGACTCCGTGGAGGAGGTCTTATCAGAGGCACGTCAACATCT |
| | TAAAGATGGCACTTGTGGCTTAGTAGAAGTTGAAAAAGGCGTTTTGCCTCAACTTGAACAGCCCTAT |
| | GTGTTCATCAAACGTTCGGATGCTCGAACTGCACCTCATGGTCATGTTATGGTTGAGCTGGTAGCAG |
| | AACTCGAAGGCATTCAGTACGGTCGTAGTGGTGAGACACTTGGTGTCCTTGTCCCTCATGTGGGCGA |
| | AATACCAGTGGCTTACCGCAAGGTTCTTCTTCGTAAGAACGGTAATAAAGGAGCTGGTGGCCATAGT |
| | TACGGCGCCGATCTAAAGTCATTTGACTTAGGCGACGAGCTTGGCACTGATCCTTATGAAGACTTTC |
| | AAGAAAACTGGAACACTAAACATAGCAGTGGTGTTACCCGTGAACTCATGCGTGAGCTTAACGGAGG |
| | GGCATACACTCGCTATGTCGATAACAACTTCTGTGGCCCTGATGGCTACCCTCTTGAGTGCATTAAA |
| | GACCTTCTAGCACGTGCTGGTAAAGCTTCATGCACTTTGTCCGAACAACTGGACTTTATTGACACTA |
| | AGAGGGGTGTATACTGCTGCCGTGAACATGAGCATGAAATTGCTTGGTACACGGAACGTTCTGAAAA |
| | GAGCTATGAATTGCAGACACCTTTTGAAATTAAATTGGCAAAGAAATTTGACACCTTCAATGGGGAA |
| | TGTCCAAATTTTGTATTTCCCTTAAATTCCATAATCAAGACTATTCAACCAAGGGTTGAAAAGAAAA |
| | AGCTTGATGGCTTTATGGGTAGAATTCGATCTGTCTATCCAGTTGCGTCACCAAATGAATGCAACCA |
| | AATGTGCCTTTCAACTCTCATGAAGTGTGATCATTGTGGTGAAACTTCATGGCAGACGGGCGATTTT |
| | GTTAAAGCCACTTGCGAATTTTGTGGCACTGAGAATTTGACTAAAGAAGGTGCCACTACTTGTGGTT |
| | ACTTACCCCAAAATGCTGTTGTTAAAATTTATTGTCCAGCATGTCACAATTCAGAAGTAGGACCTGA |
| | GCATAGTCTTGCCGAATACCATAATGAATCTGGCTTGAAAACCATTCTTCGTAAGGGTGGTCGCACT |
| | ATTGCCTTTGGAGGCTGTGTGTTCTCTTATGTTGGTTGCCATAACAAGTGTGCCTATTGGGTTCCAC |
| | GTGCTAGCGCTAACATAGGTTGTAACCATACAGGTGTTGTTGGAGAAGGTTCCGAAGGTCTTAATGA |
| | CAACCTTCTTGAAATACTCCAAAAAGAGAAAGTCAACATCAATATTGTTGGTGACTTTAAACTTAAT |
| | GAAGAGATCGCCATTATTTTGGCATCTTTTTCTGCTTCCACAAGTGCTTTTGTGGAAACTGTGAAAG |
| | GTTTGGATTATAAAGCATTCAAACAAATTGTTGAATCCTGTGGTAATTTTAAAGTTACAAAAGGAAA |
| | AGCTAAAAAAGGTGCCTGGAATATTGGTGAACAGAAATCAATACTGAGTCCTCTTTATGCATTTGCA |
| | TCAGAGGCTGCTCGTGTTGTACGATCAATTTTCTCCCGCACTCTTGAAACTGCTCAAAATTCTGTGC |
| | GTGTTTTACAGAAGGCCGCTATAACAATACTAGATGGAATTTCACAGTATTCACTGAGACTCATTGA |
| | TGCTATGATGTTCACATCTGATTTGGCTACTAACAATCTAGTTGTAATGGCCTACATTACAGGTGGT |
| | GTTGTTCAGTTGACTTCGCAGTGGCTAACTAACATCTTTGGCACTGTTTATGAAAAACTCAAACCCG |
| | TCCTTGATTGGCTTGAAGAGAAGTTTAAGGAAGGTGTAGAGTTTCTTAGAGACGGTTGGGAAATTGT |
| | TAAATTTATCTCAACCTGTGCTTGTGAAATTGTCGGTGGACAAATTGTCACCTGTGCAAAGGAAATT |
| | AAGGAGAGTGTTCAGACATTCTTTAAGCTTGTAAATAAATTTTTGGCTTTGTGTGCTGACTCTATCA |
| | TTATTGGTGGAGCTAAACTTAAAGCCTTGAATTTAGGTGAAACATTTGTCACGCACTCAAAGGGATT |
| | GTACAGAAAGTGTGTTAAATCCAGAGAAGAAACTGGCCTACTCATGCCTCTAAAAGCCCCAAAAGAA |
| | ATTATCTTCTTAGAGGGAGAAACACTTCCCACAGAAGTGTTAACAGAGGAAGTTGTCTTGAAAACTG |
| | GTGATTTACAACCATTAGAACAACCTACTAGTGAAGCTGTTGAAGCTCCATTGGTTGGTACACCAGT |
| | TTGTATTAACGGGCTTATGTTGCTCGAAATCAAAGACACAGAAAAGTACTGTGCCCTTGCACCTAAT |
| | ATGATGGTAACAAACAATACCTTCACACTCAAAGGCGGTGCACCAACAAAGGTTACTTTTGGTGATG |
| | ATACTGTGATAGAAGTGCAAGGTTACAAGAGTGTGAATATCACTTTTGAACTTGATGAAAGGATTGA |
| | TAAAGTACTTAATGAGAAGTGCTCTGCCTATACAGTTGAACTCGGTACAGAAGTAAATGAGTTCGCC |
| | TGTGTTGTGGCAGATGCTGTCATAAAAACTTTGCAACCAGTATCTGAATTACTTACACCACTGGGCA |
| | TTGATTTAGATGAGTGGAGTATGGCTACATACTACTTATTTGATGAGTCTGGTGAGTTTAAATTGGC |
| | TTCACATATGTATTGTTCTTTTTACCCTCCAGATGAGGATGAAGAAGAAGGTGATTGTGAAGAAGAA |
| | GAGTTTGAGCCATCAACTCAATATGAGTATGGTATTGAAGATGATTACCAAGGTAAACCTTTGGAAT |
| | TTGGTGCCACTTCTGCTGCTCTTCAACCTGAAGAAGAGCAAGAAGAAGATTGGTTAGATGATGATAG |
| | TCAACAAACTGTTGGTCAACAAGACGGCAGTGAGGACAATCAGACAACTACTATTCAAACAATTGTT |
| | GAGGTTCAACCTCAATTAGAGATGGAACTTACACCAGTTGTTCAGACTATTGAAGTGAATAGTTTTA |
| | GTGGTTATTTAAAACTTACTGACAATGTATACATTAAAAATGCAGACATTGTGGAAGAAGCTAAAAA |
| | GGTAAAACCAACAGTGGTTGTTAATGCAGCCAATGTTTACCTTAAACATGGAGGAGGTGTTGCAGGA |
| | GCCTTAAATAAGGCTACTAACAATGCCATGCAAGTTGAATCTGATGATTACATAGCTACTAATGGAC |
| | CACTTAAAGTGGGTGGTAGTTGTGTTTTAAGCGGACACAATCTTGCTAAACACTGTCTTCATGTTGT |
| | CGGCCCAAATGTTAACAAAGGTGAAGACATTCAACTTCTTAAGAGTGCTTATGAAAATTTTAATCAG |
| | CACGAAGTTCTACTTGCACCATTATTATCAGCTGGTATTTTTGGTGCTGACCCTATACATTCTTTAA |
| | GAGTTTGTGTAGATACTGTTCGCACAAATGTCTACTTAGCTGTCTTTGATAAAAATCTCTATGACAA |
| | ACTTGTTTTAAGCTTTTTGGAAATGAAGAGTGAAAAGCAAGTTGAACAAAAGATCGCTGAGATTCCT |
| | AAAGAGGAAGTTAAGCCATTTATAACTGAAAGTAAACCTTCAGTTGAACAGAGAAAACAAGATGATA |
| | AGAAAATCAAAGCTTGTGTTGAAGAAGTTACAACAACTCTGGAAGAAACTAAGTTCCTCACAGAAAA |
| | CTTGTTACTTTATATTGACATTAATGGCAATCTTCATCCAGATTCTGCCACTCTTGTTAGTGACATT |
| | GACATCACTTTCTTAAAGAAAGATGCTCCATATATAGTGGGTGATGTTGTTCAAGAGGGTGTTTTAA |
| | CTGCTGTGGTTATACCTACTAAAAAGGCTGGTGGCACTACTGAAATGCTAGCGAAAGCTTTGAGAAA |
| | AGTGCCAACAGACAATTATATAACCACTTACCCGGGTCAGGGTTTAAATGGTTACACTGTAGAGGAG |
| | GCAAAGACAGTGCTTAAAAAGTGTAAAAGTGCCTTTTACATTCTACCATCTATTATCTCTAATGAGA |
| | AGCAAGAAATTCTTGGAACTGTTTCTTGGAATTTGCGAGAAATGCTTGCACATGCAGAAGAAACACG |
| | CAAATTAATGCCTGTCTGTGTGGAAACTAAAGCCATAGTTTCAACTATACAGCGTAAATATAAGGGT |
| | ATTAAAATACAAGAGGGTGTGGTTGATTATGGTGCTAGATTTTACTTTTACACCAGTAAAACAACTG |
| | TAGCGTCACTTATCAACACACTTAACGATCTAAATGAAACTCTTGTTACAATGCCACTTGGCTATGT |
| | AACACATGGCTTAAATTTGGAAGAAGCTGCTCGGTATATGAGATCTCTCAAAGTGCCAGCTACAGTT |
| | TCTGTTTCTTCACCTGATGCTGTTACAGCGTATAATGGTTATCTTACTTCTTCTTCTAAAACACCTG |
| | AAGAACATTTTATTGAAACCATCTCACTTGCTGGTTCCTATAAAGATTGGTCCTATTCTGGACAATC |
| | TACACAACTAGGTATAGAATTTCTTAAGAGAGGTGATAAAAGTGTATATTACACTAGTAATCCTACC |
| | ACATTCCACCTAGATGGTGAAGTTATCACCTTTGACAATCTTAAGACACTTCTTTCTTTGAGAGAAG |
| | TGAGGACTATTAAGGTGTTTACAACAGTAGACAACATTAACCTCCACACGCAAGTTGTGGACATGTC |
| | AATGACATATGGACAACAGTTTGGTCCAACTTATTTGGATGGAGCTGATGTTACTAAAATAAAACCT |
| | CATAATTCACATGAAGGTAAAACATTTTATGTTTTACCTAATGATGACACTCTACGTGTTGAGGCTT |
| | TTGAGTACTACCACACAACTGATCCTAGTTTTCTGGGTAGGTACATGTCAGCATTAAATCACACTAA |
| | AAAGTGGAAATACCCACAAGTTAATGGTTTAACTTCTATTAAATGGGCAGATAACAACTGTTATCTT |
| | GCCACTGCATTGTTAACACTCCAACAAATAGAGTTGAAGTTTAATCCACCTGCTCTACAAGATGCTT |
| | ATTACAGAGCAAGGGCTGGTGAAGCTGCTAACTTTTGTGCACTTATCTTAGCCTACTGTAATAAGAC |
| | AGTAGGTGAGTTAGGTGATGTTAGAGAAACAATGAGTTACTTGTTTCAACATGCCAATTTAGATTCT |
| | TGCAAAAGAGTCTTGAACGTGGTGTGTAAAACTTGTGGACAACAGCAGACAACCCTTAAGGGTGTAG |
| | AAGCTGTTATGTACATGGGCACACTTTCTTATGAACAATTTAAGAAAGGTGTTCAGATACCTTGTAC |
| | GTGTGGTAAACAAGCTACACAATATCTAGTACAACAGGAGTCACCTTTTGTTATGATGTCAGCACCA |
| | CCTGCTCAGTATGAACTTAAGCATGGTACATTTACTTGTGCTAGTGAGTACACTGGTAATTACCAGT |
| | GTGGTCACTATAAACATATAACTTCTAAAGAAACTTTGTATTGCATAGACGGTGCTTTACTTACAAA |
| | GTCCTCAGAATACAAAGGTCCTATTACGGATGTTTTCTACAAAGAAAACAGTTACACAACAACCATA |
| | AAACCAGTTACTTATAAATTGGATGGTGTTGTTTGTACAGAAATTGACCCTAAGTTGGACAATTATT |
| | ATAAGAAAGACAATTCTTATTTCACAGAGCAACCAATTGATCTTGTACCAAACCAACCATATCCAAA |
| | CGCAAGCTTCGATAATTTTAAGTTTGTATGTGATAATATCAAATTTGCTGATGATTTAAACCAGTTA |
| | ACTGGTTATAAGAAACCTGCTTCAAGAGAGCTTAAAGTTACATTTTTCCCTGACTTAAATGGTGATG |
| | TGGTGGCTATTGATTATAAACACTACACACCCTCTTTTAAGAAAGGAGCTAAATTGTTACATAAACC |
| | TATTGTTTGGCATGTTAACAATGCAACTAATAAAGCCACGTATAAACCAAATACCTGGTGTATACGT |
| | TGTCTTTGGAGCACAAAACCGGTTGAAACATCAAATTCGTTTGATGTACTGAAGTCAGAGGACGCGC |
| | AGGGAATGGATAATCTTGCCTGCGAAGATCTAAAACCAGTCTCTGAAGAAGTAGTGGAAAATCCTAC |
| | CATACAGAAAGACGTTCTTGAGTGTAATGTGAAAACTACCGAAGTTGTAGGAGACATTATACTTAAA |
| | CCAGCAAATAATAGTTTAAAAATTACAGAAGAGGTTGGCCACACAGATCTAATGGCTGCTTATGTAG |
| | ACAATTCTAGTCTTACTATTAAGAAACCTAATGAATTATCTAGAGTGTTAGGTTTGAAAACCCTTGC |
| | TACTCATGGTTTAGCTGCTGTTAATAGTGTCCCTTGGGATACTATAGCTAATTATGCTAAGCCTTTT |
| | CTTAACAAAGTTGTTAGTACAACTACTAACATAGTTACACGGTGTTTAAACCGTGTTTGTACTAATT |
| | ATATGCCTTATTTCTTTACTTTATTGCTACAATTGTGTACTTTTACTAGAAGTACAAATTCTAGAAT |
| | TAAAGCATCTATGCCGACTACTATAGCAAAGAATACTGTTAAGAGTGTCGGTAAATTTTGTCTAGAG |
| | GCTTCATTTAATTATTTGAAGTCACCTAATTTTTCTAAACTGATAAATATTATAATTTGGTTTTTAC |
| | TATTAAGTGTTTGCCTAGGTTCTTTAATCTACTCAACCGCTGCTTTAGGTGTTTTAATGTCTAATTT |
| | AGGCATGCCTTCTTACTGTACTGGTTACAGAGAAGGCTATTTGAACTCTACTAATGTCACTATTGCA |
| | ACCTACTGTACTGGTTCTATACCTTGTAGTGTTTGTCTTAGTGGTTTAGATTCTTTAGACACCTATC |
| | CTTCTTTAGAAACTATACAAATTACCATTTCATCTTTTAAATGGGATTTAACTGCTTTTGGCTTAGT |
| | TGCAGAGTGGTTTTTGGCATATATTCTTTTCACTAGGTTTTTCTATGTACTTGGATTGGCTGCAATC |
| | ATGCAATTGTTTTTCAGCTATTTTGCAGTACATTTTATTAGTAATTCTTGGCTTATGTGGTTAATAA |
| | TTAATCTTGTACAAATGGCCCCGATTTCAGCTATGGTTAGAATGTACATCTTCTTTGCATCATTTTA |
| | TTATGTATGGAAAAGTTATGTGCATGTTGTAGACGGTTGTAATTCATCAACTTGTATGATGTGTTAG |
| | AAACGTAATAGAGCAACAAGAGTCGAATGTACAACTATTGTTAATGGTGTTAGAAGGTCCTTTTATG |
| | TCTATGCTAATGGAGGTAAAGGCTTTTGCAAACTACACAATTGGAATTGTGTTAATTGTGATACATT |
| | CTGTGCTGGTAGTACATTTATTAGTGATGAAGTTGCGAGAGACTTGTCACTACAGTTTAAAAGACCA |
| | ATAAATCCTACTGACCAGTCTTCTTACATCGTTGATAGTGTTACAGTGAAGAATGGTTCCATCCATC |
| | TTTACTTTGATAAAGCTGGTCAAAAGACTTATGAAAGACATTCTCTCTCTCATTTTGTTAACTTAGA |
| | CAACCTGAGAGCTAATAACACTAAAGGTTCATTGCCTATTAATGTTATAGTTTTTGATGGTAAATCA |
| | AAATGTGAAGAATCATCTGCAAAATCAGCGTCTGTTTACTACAGTCAGCTTATGTGTCAACCTATAC |
| | TGTTACTAGATCAGGCATTAGTGTCTGATGTTGGTGATAGTGCGGAAGTTGCAGTTAAAATGTTTGA |
| | TGCTTACGTTAATACGTTTTCATCAACTTTTAACGTACCAATGGAAAAACTCAAAACACTAGTTGCA |
| | ACTGCAGAAGCTGAACTTGCAAAGAATGTGTCCTTAGACAATGTCTTATCTACTTTTATTTCAGCAG |
| | CTCGGCAAGGGTTTGTTGATTCAGATGTAGAAACTAAAGATGTTGTTGAATGTCTTAAATTGTCACA |
| | TCAATCTGACATAGAAGTTACTGGCGATAGTTGTAATAACTATATGCTCACCTATAACAAAGTTGAA |
| | AACATGACACCCCGTGACCTTGGTGCTTGTATTGACTGTAGTGCGCGTCATATTAATGCGCAGGTAG |
| | CAAAAAGTCACAACATTGCTTTGATATGGAACGTTAAAGATTTCATGTCATTGTCTGAACAACTACG |
| | AAAACAAATACGTAGTGCTGCTAAAAAGAATAACTTACCTTTTAAGTTGACATGTGCAACTACTAGA |
| | CAAGTTGTTAATGTTGTAACAACAAAGATAGCACTTAAGGGTGGTAAAATTGTTAATAATTGGTTGA |
| | AGCAGTTAATTAAAGTTACACTTGTGTTCCTTTTTGTTGCTGCTATTTTCTATTTAATAACACCTGT |
| | TCATGTCATGTCTAAACATACTGACTTTTCAAGTGAAATCATAGGATACAAGGCTATTGATGGTGGT |
| | GTCACTCGTGACATAGCATCTACAGATACTTGTTTTGCTAACAAACATGCTGATTTTGACACATGGT |
| | TTAGCCAGCGTGGTGGTAGTTATACTAATGACAAAGCTTGCCCATTGATTGCTGCAGTCATAACAAG |
| | AGAAGTGGGTTTTGTCGTGCCTGGTTTGCCTGGCACGATATTACGCACAACTAATGGTGACTTTTTG |
| | CATTTCTTACCTAGAGTTTTTAGTGCAGTTGGTAACATCTGTTACACACCATCAAAACTTATAGAGT |
| | ACACTGACTTTGCAACATCAGCTTGTGTTTTGGCTGCTGAATGTACAATTTTTAAAGATGCTTCTGG |
| | TAAGCCAGTACCATATTGTTATGATACCAATGTACTAGAAGGTTCTGTTGCTTATGAAAGTTTACGC |
| | CCTGACACACGTTATGTGCTCATGGATGGCTCTATTATTCAATTTCCTAACACCTACCTTGAAGGTT |
| | CTGTTAGAGTGGTAACAACTTTTGATTCTGAGTACTGTAGGCACGGCACTTGTGAAAGATCAGAAGC |
| | TGGTGTTTGTGTATCTACTAGTGGTAGATGGGTACTTAACAATGATTATTACAGATCTTTACCAGGA |
| | GTTTTCTGTGGTGTAGATGCTGTAAATTTACTTACTAATATGTTTACACCACTAATTCAACCTATTG |
| | GTGCTTTGGACATATCAGCATCTATAGTAGCTGGTGGTATTGTAGCTATCGTAGTAACATGCCTTGC |
| | CTACTATTTTATGAGGTTTAGAAGAGCTTTTGGTGAATACAGTCATGTAGTTGCCTTTAATACTTTA |
| | CTATTCCTTATGTCATTCACTGTACTCTGTTTAACACCAGTTTACTCATTCTTACCTGGTGTTTATT |
| | CTGTTATTTACTTGTACTTGACATTTTATCTTACTAATGATGTTTCTTTTTTAGCACATATTCAGTG |
| | GATGGTTATGTTCACACCTTTAGTACCTTTCTGGATAACAATTGCTTATATCATTTGTATTTCCACA |
| | AAGCATTTCTATTGGTTCTTTAGTAATTACCTAAAGAGACGTGTAGTCTTTAATGGTGTTTCCTTTA |
| | GTACTTTTGAAGAAGCTGCGCTGTGCACCTTTTTGTTAAATAAAGAAATGTATCTAAAGTTGCGTAG |
| | TGATGTGCTATTACCTCTTACGCAATATAATAGATACTTAGCTCTTTATAATAAGTACAAGTATTTT |
| | AGTGGAGCAATGGATACAACTAGCTACAGAGAAGCTGCTTGTTGTCATCTCGCAAAGGCTCTCAATG |
| | ACTTCAGTAACTCAGGTTCTGATGTTCTTTACCAACCACCACAAACCTCTATCACCTCAGCTGTTTT |
| | GCAGAGTGGTTTTAGAAAAATGGCATTCCCATCTGGTAAAGTTGAGGGTTGTATGGTACAAGTAACT |
| | TGTGGTACAACTACACTTAACGGTCTTTGGCTTGATGACGTAGTTTACTGTCCAAGACATGTGATCT |
| | GCACCTCTGAAGACATGCTTAACCCTAATTATGAAGATTTACTCATTCGTAAGTCTAATCATAATTT |
| | CTTGGTACAGGCTGGTAATGTTCAACTCAGGGTTATTGGACATTCTATGCAAAATTGTGTACTTAAG |
| | CTTAAGGTTGATACAGCCAATCCTAAGACACCTAAGTATAAGTTTGTTCGCATTCAACCAGGACAGA |
| | CTTTTTCAGTGTTAGCTTGTTACAATGGTTCACCATCTGGTGTTTACCAATGTGCTATGAGGCCCAA |
| | TTTCACTATTAAGGGTTCATTCCTTAATGGTTCATGTGGTAGTGTTGGTTTTAACATAGATTATGAC |
| | TGTGTCTCTTTTTGTTACATGCACCATATGGAATTACCAACTGGAGTTCATGCTGGCACAGACTTAG |
| | AAGGTAACTTTTATGGACCTTTTGTTGACAGGCAAACAGCACAAGCAGCTGGTACGGACACAACTAT |
| | TACAGTTAATGTTTTAGCTTGGTTGTACGCTGCTGTTATAAATGGAGACAGGTGGTTTCTCAATCGA |
| | TTTACCACAACTCTTAATGACTTTAACCTTGTGGCTATGAAGTACAATTATGAACCTCTAACACAAG |
| | ACCATGTTGACATACTAGGACCTCTTTCTGCTCAAACTGGAATTGCCGTTTTAGATATGTGTGCTTC |
| | ATTAAAAGAATTACTGCAAAATGGTATGAATGGACGTACCATATTGGGTAGTGCTTTATTAGAAGAT |
| | GAATTTACACCTTTTGATGTTGTTAGACAATGCTCAGGTGTTACTTTCCAAAGTGCAGTGAAAAGAA |
| | CAATCAAGGGTACACACCACTGGTTGTTACTCACAATTTTGACTTCACTTTTAGTTTTAGTCCAGAG |
| | TACTCAATGGTCTTTGTTCTTTTTTTTGTATGAAAATGCCTTTTTACCTTTTGCTATGGGTATTATT |
| | GCTATGTCTGCTTTTGCAATGATGTTTGTCAAACATAAGCATGCATTTCTCTGTTTGTTTTTGTTAC |
| | CTTCTCTTGCCACTGTAGCTTATTTTAATATGGTCTATATGCCTGCTAGTTGGGTGATGCGTATTAT |
| | GACATGGTTGGATATGGTTGATACTAGTTTGAAGCTAAAAGACTGTGTTATGTATGCATCAGCTGTA |
| | GTGTTACTAATCCTTATGACAGCAAGAACTGTGTATGATGATGGTGCTAGGAGAGTGTGGACACTTA |
| | TGAATGTCTTGACACTCGTTTATAAAGTTTATTATGGTAATGCTTTAGATCAAGCCATTTCCATGTG |
| | GGCTCTTATAATCTCTGTTACTTCTAACTACTCAGGTGTAGTTACAACTGTCATGTTTTTGGCCAGA |
| | GGTATTGTTTTTATGTGTGTTGAGTATTGCCCTATTTTCTTCATAACTGGTAATACACTTCAGTGTA |
| | TAATGCTAGTTTATTGTTTCTTAGGCTATTTTTGTACTTGTTACTTTGGCCTCTTTTGTTTACTCAA |
| | CCGCTACTTTAGACTGACTCTTGGTGTTTATGATTACTTAGTTTCTACACAGGAGTTTAGATATATG |
| | AATTCACAGGGACTACTCCCACCCAAGAATAGCATAGATGCCTTCAAACTCAACATTAAATTGTTGG |
| | GTGTTGGTGGCAAACCTTGTATCAAAGTAGCCACTGTACAGTCTAAAATGTCAGATGTAAAGTGCAC |
| | ATCAGTAGTCTTACTCTCAGTTTTGCAACAACTCAGAGTAGAATCATCATCTAAATTGTGGGCTCAA |
| | TGTGTCCAGTTACACAATGACATTCTCTTAGCTAAAGATACTACTGAAGCCTTTGAAAAAATGGTTT |
| | CACTACTTTCTGTTTTGCTTTCCATGCAGGGTGCTGTAGACATAAACAAGCTTTGTGAAGAAATGCT |
| | GGACAACAGGGCAACCTTACAAGCTATAGCCTCAGAGTTTAGTTCCCTTCCATCATATGCAGCTTTT |
| | GCTACTGCTCAAGAAGCTTATGAGCAGGCTGTTGCTAATGGTGATTCTGAAGTTGTTCTTAAAAAGT |
| | TGAAGAAGTCTTTGAATGTGGCTAAATCTGAATTTGACCGTGATGCAGCCATGCAACGTAAGTTGGA |
| | AAAGATGGCTGATCAAGCTATGACCCAAATGTATAAACAGGCTAGATCTGAGGACAAGAGGGCAAAA |
| | GTTACTAGTGCTATGCAGACAATGCTTTTCACTATGCTTAGAAAGTTGGATAATGATGCACTCAACA |
| | ACATTATCAACAATGCAAGAGATGGTTGTGTTCCCTTGAACATAATACCTCTTACAACAGCAGCCAA |
| | ACTAATGGTTGTCATACCAGACTATAACACATATAAAAATACGTGTGATGGTACAACATTTACTTAT |
| | GCATCAGCATTGTGGGAAATCCAACAGGTTGTAGATGCAGATAGTAAAATTGTTCAACTTAGTGAAA |
| | TTAGTATGGACAATTCACCTAATTTAGCATGGCCTCTTATTGTAACAGCTTTAAGGGCCAATTCTGC |
| | TGTCAAATTACAGAATAATGAGCTTAGTCCTGTTGCACTACGACAGATGTCTTGTGCTGCCGGTACT |
| | ACACAAACTGCTTGCACTGATGACAATGCGTTAGCTTATTACAACACAACAAAGGGAGGTAGGTTTG |
| | TACTTGCACTGTTATCCGATTTACAGGATTTGAAATGGGCTAGATTCCCTAAGAGTGATGGAACTGG |
| | TACTATCTATACAGAACTGGAACCACCTTGTAGGTTTGTTACAGACACACCTAAAGGTCCTAAAGTG |
| | AAGTATTTATACTTTATTAAAGGATTAAACAACCTAAATAGAGGTATGGTACTTGGTAGTTTAGCTG |
| | CCACAGTACGTCTACAAGCTGGTAATGCAACAGAAGTGCCTGCCAATTCAACTGTATTATCTTTCTG |
| | TGCTTTTGCTGTAGATGCTGCTAAAGCTTACAAAGATTATCTAGCTAGTGGGGGACAACCAATCACT |
| | AATTGTGTTAAGATGTTGTGTACACACACTGGTACTGGTCAGGCAATAACAGTTACACCGGAAGCCA |
| | ATATGGATCAAGAATCCTTTGGTGGTGCATCGTGTTGTCTGTACTGCCGTTGCCACATAGATCATCC |
| | AAATCCTAAAGGATTTTGTGACTTAAAAGGTAAGTATGTACAAATACCTACAACTTGTGCTAATGAC |
| | CCTGTGGGTTTTACACTTAAAAACACAGTCTGTACCGTCTGCGGTATGTGGAAAGGTTATGGCTGTA |
| | GTTGTGATCAACTCCGCGAACCCATGCTTCAGTCAGCTGATGCACAATCGTTTTTAAACGGGTTTGC |
| | GGTGTAAGTGCAGCCCGTCTTACACCGTGCGGCACAGGCACTAGTACTGATGTCGTATACAGGGCTT |
| | TTGACATCTACAATGATAAAGTAGCTGGTTTTGCTAAATTCCTAAAAACTAATTGTTGTCGCTTCCA |
| | AGAAAAGGACGAAGATGACAATTTAATTGATTCTTACTTTGTAGTTAAGAGACACACTTTCTCTAAC |
| | TACCAACATGAAGAAACAATTTATAATTTACTTAAGGATTGTCCAGCTGTTGCTAAACATGACTTCT |
| | TTAAGTTTAGAATAGACGGTGACATGGTACCACATATATCACGTCAACGTCTTACTAAATACACAAT |
| | GGCAGACCTCGTCTATGCTTTAAGGCATTTTGATGAAGGTAATTGTGATACATTAAAAGAAATACTT |
| | GTCACATACAATTGTTGTGATGATGATTATTTCAATAAAAAGGACTGGTATGATTTTGTAGAAAACC |
| | CAGATATATTACGCGTATACGCCAACTTAGGTGAACGTGTACGCCAAGCTTTGTTAAAAACAGTACA |
| | ATTCTGTGATGCCATGCGAAATGCTGGTATTGTTGGTGTACTGACATTAGATAATCAAGATCTCAAT |
| | GGTAACTGGTATGATTTCGGTGATTTCATACAAACCACGCCAGGTAGTGGAGTTCCTGTTGTAGATT |
| | CTTATTATTCATTGTTAATGCCTATATTAACCTTGACCAGGGCTTTAACTGCAGAGTCACATGTTGA |
| | CACTGACTTAACAAAGCCTTACATTAAGTGGGATTTGTTAAAATATGACTTCACGGAAGAGAGGTTA |
| | AAACTCTTTGACCGTTATTTTAAATATTGGGATCAGACATACCACCCAAATTGTGTTAACTGTTTGG |
| | ATGACAGATGCATTCTGCATTGTGCAAACTTTAATGTTTTATTCTCTACAGTGTTCCCACTTACAAG |
| | TTTTGGACCACTAGTGAGAAAAATATTTGTTGATGGTGTTCCATTTGTAGTTTCAACTGGATACCAC |
| | TTCAGAGAGCTAGGTGTTGTACATAATCAGGATGTAAACTTACATAGCTCTAGACTTAGTTTTAAGG |
| | AATTACTTGTGTATGCTGCTGACCCTGCTATGCACGCTGCTTCTGGTAATCTATTACTAGATAAACG |
| | CACTACGTGCTTTTCAGTAGCTGCACTTACTAACAATGTTGCTTTTCAAACTGTCAAACCCGGTAAT |
| | TTTAACAAAGACTTCTATGACTTTGCTGTGTCTAAGGGTTTCTTTAAGGAAGGAAGTTCTGTTGAAT |
| | TAAAACACTTCTTCTTTGCTCAGGATGGTAATGCTGCTATCAGCGATTATGACTACTATCGTTATAA |
| | TCTACCAACAATGTGTGATATCAGACAACTACTATTTGTAGTTGAAGTTGTTGATAAGTACTTTGAT |
| | TGTTACGATGGTGGCTGTATTAATGCTAACCAAGTCATCGTCAACAACCTAGACAAATCAGCTGGTT |
| | TTCCATTTAATAAATGGGGTAAGGCTAGACTTTATTATGATTCAATGAGTTATGAGGATCAAGATGC |
| | ACTTTTCGCATATACAAAACGTAATGTCATCCCTACTATAACTCAAATGAATCTTAAGTATGCCATT |
| | AGTGCAAAGAATAGAGCTCGCACCGTAGCTGGTGTCTCTATCTGTAGTACTATGACCAATAGACAGT |
| | TTCATCAAAAATTATTGAAATCAATAGCCGCCACTAGAGGAGCTACTGTAGTAATTGGAACAAGCAA |
| | ATTCTATGGTGGTTGGCACAACATGTTAAAAACTGTTTATAGTGATGTAGAAAACCCTCACCTTATG |
| | GGTTGGGATTATCCTAAATGTGATAGAGCCATGCCTAACATGCTTAGAATTATGGCCTCACTTGTTC |
| | TTGCTCGCAAACATACAACGTGTTGTAGCTTGTCACACCGTTTCTATAGATTAGCTAATGAGTGTGC |
| | TCAAGTATTGAGTGAAATGGTCATGTGTGGCGGTTCACTATATGTTAAACCAGGTGGAACCTCATCA |
| | GGAGATGCCACAACTGCTTATGCTAATAGTGTTTTTAACATTTGTCAAGCTGTCACGGCCAATGTTA |
| | ATGCACTTTTATCTACTGATGGTAACAAAATTGCCGATAAGTATGTCCGCAATTTACAACACAGACT |
| | TTATGAGTGTCTCTATAGAAATAGAGATGTTGACACAGACTTTGTGAATGAGTTTTACGCATATTTG |
| | CGTAAACATTTCTCAATGATGATACTCTCTGACGATGCTGTTGTGTGTTTCAATAGCACTTATGCAT |
| | CTCAAGGTCTAGTGGCTAGCATAAAGAACTTTAAGTCAGTTCTTTATTATCAAAACAATGTTTTTAT |
| | GTCTGAAGCAAAATGTTGGACTGAGACTGACCTTACTAAAGGACCTCATGAATTTTGCTCTCAACAT |
| | ACAATGCTAGTTAAACAGGGTGATGATTATGTGTACCTTCCTTACCCAGATCCATCAAGAATCCTAG |
| | GGGCCGGCTGTTTTGTAGATGATATCGTAAAAACAGATGGTACACTTATGATTGAACGGTTCGTGTC |
| | TTTAGCTATAGATGCTTACCCACTTACTAAACATCCTAATCAGGAGTATGCTGATGTCTTTCATTTG |
| | TACTTACAATACATAAGAAAGCTACATGATGAGTTAACAGGACACATGTTAGACATGTATTCTGTTA |
| | TGCTTACTAATGATAACACTTCAAGGTATTGGGAACCTGAGTTTTATGAGGCTATGTACACACCGCA |
| | TACAGTCTTACAGGCTGTTGGGGCTTGTGTTCTTTGCAATTCACAGACTTCATTAAGATGTGGTGCT |
| | TGCATACGTAGACCATTCTTATGTTGTAAATGCTGTTACGACCATGTCATATCAACATCACATAAAT |
| | TAGTCTTGTCTGTTAATCCGTATGTTTGCAATGCTCCAGGTTGTGATGTCACAGATGTGACTCAACT |
| | TTACTTAGGAGGTATGAGCTATTATTGTAAATCACATAAACCACCCATTAGTTTTCCATTGTGTGCT |
| | AATGGACAAGTTTTTGGTTTATATAAAAATACATGTGTTGGTAGCGATAATGTTACTGACTTTAATG |
| | CAATTGCAACATGTGACTGGACAAATGCTGGTGATTACATTTTAGCTAACACCTGTACTGAAAGACT |
| | CAAGCTTTTTGCAGCAGAAACGCTCAAAGCTACTGAGGAGACATTTAAACTGTCTTATGGTATTGCT |
| | ACTGTACGTGAAGTGCTGTCTGACAGAGAATTACATCTTTCATGGGAAGTTGGTAAACCTAGACCAC |
| | CACTTAACCGAAATTATGTCTTTACTGGTTATCGTGTAACTAAAAACAGTAAAGTACAAATAGGAGA |
| | GTACACCTTTGAAAAAGGTGACTATGGTGATGCTGTTGTTTACCGAGGTACAACAACTTACAAATTA |
| | AATGTTGGTGATTATTTTGTGCTGACATCACATACAGTAATGCCATTAAGTGCACCTACACTAGTGC |
| | CACAAGAGCACTATGTTAGAATTACTGGCTTATACCCAACACTCAATATCTCAGATGAGTTTTCTAG |
| | CAATGTTGCAAATTATCAAAAGGTTGGTATGCAAAAGTATTCTACACTCCAGGGACCACCTGGTACT |
| | GGTAAGAGTCATTTTGCTATTGGCCTAGCTCTCTACTACCCTTCTGCTCGCATAGTGTATACAGCTT |
| | GCTCTCATGCCGCTGTTGATGCACTATGTGAGAAGGCATTAAAATATTTGCCTATAGATAAATGTAG |
| | TAGAATTATACCTGCACGTGCTCGTGTAGATTGTTTTGATAAATTCAAAGTGAATTCAACATTAGAA |
| | CAGTATGTCTTTTGTACTGTAAATGCATTGCCTGAGACGACAGCAGATATAGTTGTCTTTGATGAAA |
| | TTTCAATGGCCACAAATTATGATTTGAGTGTTGTCAATGCCAGATTACGTGCTAAGCACTATGTGTA |
| | CATTGGCGACCCTGCTCAATTACCTGCACCACGCACATTGCTAACTAAGGGCACACTAGAACCAGAA |
| | TATTTCAATTCAGTGTGTAGACTTATGAAAACTATAGGTCCAGACATGTTCCTCGGAACTTGTCGGC |
| | GTTGTCCTGCTGAAATTGTTGACACTGTGAGTGCTTTGGTTTATGATAATAAGCTTAAAGCACATAA |
| | AGACAAATCAGCTCAATGCTTTAAAATGTTTTATAAGGGTGTTATCACGCATGATGTTTCATCTGCA |
| | ATTAACAGGCCACAAATAGGCGTGGTAAGAGAATTCCTTACACGTAACCCTGCTTGGAGAAAAGCTG |
| | TCTTTATTTCACCTTATAATTCACAGAATGCTGTAGCCTCAAAGATTTTGGGACTACCAACTCAAAC |
| | TGTTGATTCATCACAGGGCTCAGAATATGACTATGTCATATTCACTCAAACCACTGAAACAGCTCAC |
| | TCTTGTAATGTAAACAGATTTAATGTTGCTATTACCAGAGCAAAAGTAGGCATACTTTGCATAATGT |
| | CTGATAGAGACCTTTATGACAAGTTGCAATTTACAAGTCTTGAAATTCCACGTAGGAATGTGGCAAC |
| | TTTACAAGCTGAAAATGTAACAGGACTCTTTAAAGATTGTAGTAAGGTAATCACTGGGTTACATCCT |
| | ACACAGGCACCTACACACCTCAGTGTTGACACTAAATTCAAAACTGAAGGTTTATGTGTTGACATAC |
| | CTGGCATACCTAAGGACATGACCTATAGAAGACTCATCTCTATGATGGGTTTTAAAATGAATTATCA |
| | AGTTAATGGTTACCCTAACATGTTTATCACCCGCGAAGAAGCTATAAGACATGTACGTGCATGGATT |
| | GGCTTCGATGTCGAGGGGTGTCATGCTACTAGAGAAGCTGTTGGTACCAATTTACCTTTACAGCTAG |
| | GTTTTTCTACAGGTGTTAACCTAGTTGCTGTACCTACAGGTTATGTTGATACACCTAATAATACAGA |
| | TTTTTCCAGAGTTAGTGCTAAACCACCGCCTGGAGATCAATTTAAACACCTCATACCACTTATGTAC |
| | AAAGGACTTCCTTGGAATGTAGTGCGTATAAAGATTGTACAAATGTTAAGTGACACACTTAAAAATC |
| | TCTCTGACAGAGTCGTATTTGTCTTATGGGCACATGGCTTTGAGTTGACATCTATGAAGTATTTTGT |
| | GAAAATAGGACCTGAGCGCACCTGTTGTCTATGTGATAGACGTGCCACATGCTTTTCCACTGCTTCA |
| | GACACTTATGCCTGTTGGCATCATTCTATTGGATTTGATTACGTCTATAATCCGTTTATGATTGATG |
| | TTCAACAATGGGGTTTTACAGGTAACCTACAAAGCAACCATGATCTGTATTGTCAAGTCCATGGTAA |
| | TGCACATGTAGCTAGTTGTGATGCAATCATGACTAGGTGTCTAGCTGTCCACGAGTGCTTTGTTAAG |
| | CGTGTTGACTGGACTATTGAATATCCTATAATTGGTGATGAGCTGAAGATTAATGCGGCTTGTAGAA |
| | AGGTTCAACACATGGTTGTTAAAGCTGCATTATTAGCAGACAAATTCCCAGTTCTTCACGACATTGG |
| | TAACCCTAAAGCTATTAAGTGTGTACCTCAAGCTGATGTAGAATGGAAGTTCTATGATGCACAGCCT |
| | TGTAGTGACAAAGCTTATAAAATAGAAGAATTATTCTATTCTTATGCCACACATTCTGACAAATTCA |
| | CAGATGGTGTATGCCTATTTTGGAATTGCAATGTCGATAGATATCCTGCTAATTCCATTGTTTGTAG |
| | ATTTGACACTAGAGTGCTATCTAACCTTAACTTGCCTGGTTGTGATGGTGGCAGTTTGTATGTAAAT |
| | AAACATGCATTCCACACACCAGCTTTTGATAAAAGTGCTTTTGTTAATTTAAAACAATTACCATTTT |
| | TCTATTACTCTGACAGTCCATGTGAGTCTCATGGAAAACAAGTAGTGTCAGATATAGATTATGTACC |
| | ACTAAAGTCTGCTACGTGTATAACACGTTGCAATTTAGGTGGTGCTGTCTGTAGACATCATGCTAAT |
| | GAGTACAGATTGTATCTCGATGCTTATAACATGATGATCTCAGCTGGCTTTAGCTTGTGGGTTTACA |
| | AACAATTTGATACTTATAACCTCTGGAACACTTTTACAAGACTTCAGAGTTTAGAAAATGTGGCTTT |
| | TAATGTTGTAAATAAGGGACACTTTGATGGACAACAGGGTGAAGTACCAGTTTCTATCATTAATAAC |
| | ACTGTTTACACAAAAGTTGATGGTGTTGATGTAGAATTGTTTGAAAATAAAACAACATTACCTGTTA |
| | ATGTAGCATTTGAGCTTTGGGCTAAGCGCAACATTAAACCAGTMCCAGAGGTGAAAATACTCAATAA |
| | TTTGGGTGTGGACATTGCTGCTAATACTGTGATCTGGGACTACAAAAGAGATGCTCCAGCACATATA |
| | TCTACTATTGGTGTTTGTTCTATGACTGACATAGCCAAGAAACCAACTGAAACGATTTGTGCACCAC |
| | TCACTGTCTTTTTTGATGGTAGAGTTGATGGTCAAGTAGACTTATTTAGAAATGCCCGTAATGGTGT |
| | TCTTATTACAGAAGGTAGTGTTAAAGGTTTACAACCATCTGTAGGTCCCAAACAAGCTAGTCTTAAT |
| | GGAGTCACATTAATTGGAGAAGCCGTAAAAACACAGTTCAATTATTATAAGAAAGTTGATGGTGTTG |
| | TCCAACAATTACCTGAAACTTACTTTACTCAGAGTAGAAATTTACAAGAATTTAAACCCAGGAGTCA |
| | AATGGAAATTGATTTCTTAGAATTAGCTATGGATGAATTCATTGAACGGTATAAATTAGAAGGCTAT |
| | GCCTTCGAACATATCGTTTATGGAGATTTTAGTCATAGTCAGTTAGGTGGTTTACATCTACTGATTG |
| | GACTAGCTAAACGTTTTAAGGAATCACCTTTTGAATTAGAAGATTTTATTCCTATGGACAGTACAGT |
| | TAAAAACTATTTCATAACAGATGCGCAAACAGGTTCATCTAAGTGTGTGTGTTCTGTTATTGATTTA |
| | TTACTTGATGATTTTGTTGAAATAATAAAATCCCAAGATTTATCTGTAGTTTCTAAGGTTGTCAAAG |
| | TGACTATTGACTATACAGAAATTTCATTTATGCTTTGGTGTAAAGATGGCCATGTAGAAACATTTTA |
| | CCCAAAATTACAATCTAGTCAAGCGTGGCAACCGGGTGTTGCTATGCCTAATCTTTACAAAATGCAA |
| | AGAATGCTATTAGAAAAGTGTGACCTTCAAAATTATGGTGATAGTGCAACATTACCTAAAGGCATAA |
| | TGATGAATGTCGCAAAATATACTCAACTGTGTCAATATTTAAACACATTAACATTAGCTGTACCCTA |
| | TAATATGAGAGTTATACATTTTGGTGCTGGTTCTGATAAAGGAGTTGCACCAGGTACAGCTGTTTTA |
| | AGACAGTGGTTGCCTACGGGTACGCTGCTTGTCGATTCAGATCTTAATGACTTTGTCTCTGATGCAG |
| | ATTCAACTTTGATTGGTGATTGTGCAACTGTACATACAGCTAATAAATGGGATCTCATTATTAGTGA |
| | TATGTACGACCCTAAGACTAAAAATGTTACAAAAGAAAATGACTCTAAAGAGGGTTTTTTCACTTAC |
| | ATTTGTGGGTTTATACAACAAAAGCTAGCTCTTGGAGGTTCCGTGGCTATAAAGATAACAGAACATT |
| | CTTGGAATGCTGATCTTTATAAGCTCATGGGACACTTCGCATGGTGGACAGCCTTTGTTACTAATGT |
| | GAATGCGTCATCATCTGAAGCATTTTTAATTGGATGTAATTATCTTGGCAAACCACGCGAACAAATA |
| | GATGGTTATGTCATGCATGCAAATTACATATTTTGGAGGAATACAAATCCAATTCAGTTGTCTTCCT |
| | ATTCTTTATTTGACATGAGTAAATTTCCCCTTAAATTAAGGGGTACTGCTGTTATGTCTTTAAAAGA |
| | AGGTCAAATCAATGATATGATTTTATCTCTTCTTAGTAAAGGTAGACTTATAATTAGAGAAAACAAC |
| | AGAGTTGTTATTTCTAGTGATGTTCTTGTTAACAACTAAACGAACAATGTTTGTTTTTCTTGTTTTA |
| | TTGCCACTAGTCTCTAGTCAGTGTGTTAATTTTACAAACAGAACTCAATTACCCTCTGCATACACTA |
| | ATTCTTTCACACGTGGTGTTTATTACCCTGACAAAGTTTTCAGATCCTCAGTTTTACATTCAACTCA |
| | GGACTTGTTCTTACCTTTCTTTTCCAATGTTACTTGGTTCCATGCTATACATGTCTCTGGGACCAAT |
| | GGTACTAAGAGGTTTGATAACCCTGTCCTACCATTTAATGATGGTGTTTATTTTGCTTCCACTGAGA |
| | AGTCTAACATAATAAGAGGCTGGATTTTTGGTACTACTTTAGATTCGAAGACCCAGTCCCTACTTAT |
| | TGTTAATAACGCTACTAATGTTGTTATTAAAGTCTGTGAATTTCAATTTTGTAATTATCCATTTTTG |
| | GGTGTTTATTACCACAAAAACAACAAAAGTTGGATGGAAAGTGAGTTCAGAGTTTATTCTAGTGCGA |
| | ATAATTGCACTTTTGAATATGTCTCTCAGCCTTTTCTTATGGACCTTGAAGGAAAACAGGGTAATTT |
| | CAAAAATCTTAGTGAATTTGTGTTTAAGAATATTGATGGTTATTTTAAAATATATTCTAAGCACACG |
| | CCTATTAATTTAGTGCGTGATCTCCCTCAGGGTTTTTCGGCTTTAGAACCATTGGTAGATTTGCCAA |
| | TAGGTATTAACATCACTAGGTTTCAAACTTTACTTGCTTTACATAGAAGTTATTTGACTCCTGGTGA |
| | TTCTTCTTCAGGTTGGACAGCTGGTGCTGCAGCTTATTATGTGGGTTATCTTCAACCTAGGACTTTT |
| | CTATTAAAATATAATGAAAATGGAACCATTACAGATGCTGTAGACTGTGCACTTGACCCTCTCTCAG |
| | AAACAAAGTGTACGTTGAAATCCTTCACTGTAGAAAAAGGAATCTATCAAACTTCTAACTTTAGAGT |
| | CCAACCAACAGAATCTATTGTTAGATTTCCTAATATTACAAACTTGTGCCCTTTTGGTGAAGTTTTT |
| | AACGCCACCAGATTTGCATCTGTTTATGCTTGGAACAGGAAGAGAATCAGCAACTGTGTTGCTGATT |
| | ATTCTGTCCTATATAATTCCGCATCATTTTCCACTTTTAAGTGTTATGGAGTGTCTCCTACTAAATT |
| | AAATGATCTCTGCTTTACTAATGTCTATGCAGATTCATTTGTAATTAGAGGTGATGAAGTCAGACAA |
| | ATCGCTCCAGGGCAAACTGGAACGATTGCTGATTATAATTATAAATTACCAGATGATTTTACAGGCT |
| | GCGTTATAGCTTGGAATTCTAACAATCTTGATTCTAAGGTTGGTGGTAATTATAATTACCTGTATAG |
| | ATTGTTTAGGAAGTCTAATCTCAAACCTTTTGAGAGAGATATTTCAACTGAAATCTATCAGGCCGGT |
| | AGCACACCTTGTAATGGTGTTAAAGGTTTTAATTGTTACTTTCCTTTACAATCATATGGTTTCCAAC |
| | CCACTTATGGTGTTGGTTACCAACCATACAGAGTAGTAGTACTTTCTTTTGAACTTCTACATGCACC |
| | AGCAACTGTTTGTGGACCTAAAAAGTCTACTAATTTGGTTAAAAACAAATGTGTCAATTTCAACTTC |
| | AATGGTTTAACAGGCACAGGTGTTCTTACTGAGTCTAACAAAAAGTTTCTGCCTTTCCAACAATTTG |
| | GCAGAGACATTGCTGACACTACTGATGCTGTCCGTGATCCACAGACACTTGAGATTCTTGACATTAC |
| | ACCATGTTCTTTTGGTGGTGTCAGTGTTATAACACCAGGAACAAATACTTCTAACCAGGTTGCTGTT |
| | CTTTATCAGGGTGTTAACTGCACAGAAGTCCCTGTTGCTATTCATGCAGATCAACTTACTCCTACTT |
| | GGCGTGTTTATTCTACAGGTTCTAATGTTTTTCAAACACGTGCAGGCTGTTTAATAGGGGCTGAATA |
| | TGTCAACAACTCATATGAGTGTGACATACCCATTGGTGCAGGTATATGCGCTAGTTATCAGACTCAG |
| | ACTAATTCTCCTCGGCGGGCACGTAGTGTAGCTAGTCAATCCATCATTGCCTACACTATGTCACTTG |
| | GTGCAGAAAATTCAGTTGCTTACTCTAATAACTCTATTGCCATACCCACAAATTTTACTATTAGTGT |
| | TAGCACAGAAATTCTACCAGTGTCTATGACCAAGACATCAGTAGATTGTACAATGTACATTTGTGGT |
| | GATTCAACTGAATGCAGCAATCTTTTGTTGCAATATGGCAGTTTTTGTACACAATTAAACCGTGCTT |
| | TAACTGGAATAGCTGTTGAACAAGACAAAAACACCCAAGAAGTTTTTGCACAAGTCAAACAAATTTA |
| | CAAAACACCACCAATTAAAGATTTTGGTGGTTTTAATTTTTCACAAATATTAGCAGATCCATCAAAA |
| | CCAAGCAAGAGGTCATTTATTGAAGATCTACTTTTCAACAAAGTGACACTTGCAGATGCTGGCTTCA |
| | TCAAACAATATGGTGATTGCCTTGGTGATATTGCTGCTAGAGACCTCATTTGTGCACAAAAGTTTAA |
| | CGGCCTTACTGTTTTGCCACCTTTGCTCACAGATGAAATGATTGCTCAATACACTTCTGCACTGTTA |
| | GCGGGTACAATCACTTCTGGTTGGACCTTTGGTGCAGGTGCTGCATTACAAATACCATTTGCTATGC |
| | AAATGGCTTATAGGTTTAATGGTATTGGAGTTACACAGAATGTTCTCTATGAGAACCAAAAATTGAT |
| | TGCCAACCAATTTAATAGTGCTATTGGCAAAATTCAAGACTCACTTTCTTCCACAGCAAGTGCACTT |
| | GGAAAACTTCAAGATGTGGTCAACCAAAATGCACAAGCTTTAAACACGCTTGTTAAACAACTTAGCT |
| | CCAATTTTGGTGCAATTTCAAGTGTTTTAAATGATATCCTTTCACGTCTTGACAAAGTTGAGGCTGA |
| | AGTGCAAATTGATAGGTTGATCACAGGCAGACTTCAAAGTTTGCAGACATATGTGACTCAACAATTA |
| | ATTAGAGCTGCAGAAATCAGAGCTTCTGCTAATCTTGCTGCTATTAAAATGTCAGAGTGTGTACTTG |
| | GACAATCAAAAAGAGTTGATTTTTGTGGAAAGGGCTATCATCTTATGTCCTTCCCTCAGTCAGCACC |
| | TCATGGTGTAGTCTTCTTGCATGTGACTTATGTCCCTGCACAAGAAAAGAACTTCACAACTGCTCCT |
| | GCCATTTGTCATGATGGAAAAGCACACTTTCCTCGTGAAGGTGTCTTTGTTTCAAATGGCACACACT |
| | GGTTTGTAACACAAAGGAATTTTTATGAACCACAAATCATTACTACAGACAACACATTTGTGTCTGG |
| | TAACTGTGATGTTGTAATAGGAATTGTCAACAACACAGTTTATGATCCTTTGCAACCTGAATTAGAC |
| | TCATTCAAGGAGGAGTTAGATAAATATTTTAAGAATCATACATCACCAGATGTTGATTTAGGTGACA |
| | TCTCTGGCATTAATGCTTCATTTGTAAACATTCAAAAAGAAATTGACCGCCTCAATGAGGTTGCCAA |
| | GAATTTAAATGAATCTCTCATCGATCTCCAAGAACTTGGAAAGTATGAGCAGTATATAAAATGGCCA |
| | TGGTACATTTGGCTAGGTTTTATAGCTGGCTTGATTGCCATAGTAATGGTGACAATTATGCTTTGCT |
| | GTATGACCAGTTGCTGTAGTTGTCTCAAGGGCTGTTGTTCTTGTGGATCCTGCTGCAAATTTGATGA |
| | AGACGACTCTGAGCCAGTGCTCAAAGGAGTCAAATTACATTACACATAAACGAACTTATGGATTTGT |
| | TTATGAGAATCTTCACAATTGGAACTGTAACTTTGAAGCAAGGTGAAATCAAGGATGCTACTCCTTC |
| | AGATTTTGTTCGCGCTACTGCAACGATACCGATACAAGCCTCACTCCCTTTCGGATGGCTTATTGTT |
| | GGCGTTGCACTTCTTGCTGTTTTTCAGAGCGCTTCCAAAATCATAACCCTCAAAAAGAGATGGCAAC |
| | TAGCACTCTCCAAGGGTGTTCACTTTGTTTGCAACTTGCTGTTGTTGTTTGTAACAGTTTACTCACA |
| | CCTTTTGCTCGTTGCTGCTGGCCTTGAAGCCCCTTTTCTCTATCTTTATGCTTTAGTCTACTTCTTG |
| | CAGAGTATAAACTTTGTAAGAATAATAATGAGGCTTTGGCTTTGCTGGAAATGCCGTTCCAAAAACC |
| | CATTACTTTATGATGCCAACTATTTTCTTTGCTGGCATACTAATTGTTACGACTATTGTATACCTTA |
| | CAATAGTGTAACTTCTTCAATTGTCATTACTTCAGGTGATGGCACAACAAGTCCTATTTCTGAACAT |
| | GACTACCAGATTGGTGGTTATACTGAAAAATGGGAATCTGGAGTAAAAGACTGTGTTGTATTACACA |
| | GTTACTTTACTTCAGACTATTACCAGCTGTACTCAACTCAATTGAGTACAGACACTGGTGTTGAACA |
| | TGTTACCTTCTTCATCTACAATAAAATTGTTGATGAGCCTGAAGAACATGTCCAAATTCACACAATC |
| | GACGGTTCACCCGGAGTTGTTAATCCAGTAATGGAACCAATTTATGATGAACCGACGACGACTACTA |
| | GCGTGCCTTTGTAAGCACAAGCTGATGAGTACGAACTTATGTACTCATTCGTTTCGGAAGAGACAGG |
| | TACGTTAATAGTTAATAGCGTACTTCTTTTTCTTGCTTTCGTGGTATTCTTGCTAGTTACACTAGCC |
| | ATCCTTACTGCGCTTCGATTGTGTGCGTACTGCTGCAATATTGTTAACGTGAGTCTTGTAAAACCTT |
| | CTTTTTACGTTTACTCTCGTGTTAAAAATCTGAATTCTTCTAGAGTTCCTGATCTTCTGGTCTAAAC |
| | GAACTAAATATTATATTAGTTTTTCTGTTTGGAACTTTAATTTTAGCCATGGCAGATTCCAACGGTA |
| | CTATTACCGTTGAAGAGCTTAAAAAGCTCCTTGAACAATGGAACCTAGTAATAGGTTTCCTATTCCT |
| | TACATGGATTTGTCTTCTACAATTTGCCTATGCCAACAGGAATAGGTTTTTGTATATAATTAAGTTA |
| | ATTTTCCTCTGGCTGTTATGGCCAGTAACTTTAGCTTGTTTTGTGCTTGCTGCTGTTTACAGAATAA |
| | ATTGGATCACCGGTGGAATTGCTATCGCAATGGCTTGTCTTGTAGGCTTGATGTGGCTCAGCTACTT |
| | CATTGCTTCTTTCAGACTGTTTGCGCGTACGCGTTCCATGTGGTCATTCAATCCAGAAACTAACATT |
| | CTTCTCAACGTGCCACTCCATGGCACTATTCTGACCAGACCGCTTCTAGAAAGTGAACTCGTAATCG |
| | GAGCTGTGATCCTTCGTGGACATCTTCGTATTGCTGGACACCATCTAGGACGCTGTGACATCAAGGA |
| | CCTGCCTAAAGAAATCACTGTTGCTACATCACGAACGCTTTCTTATTACAAATTGGGAGCTTCGCAG |
| | CGTGTAGCAGGTGACTCAGGTTTTGCTGCATACAGTCGCTACAGGATTGGCAACTATAAATTAAACA |
| | CAGACCATTCCAGTAGCAGTGACAATATTGCTTTGCTTGTACAGTAAGTGACAACAGATGTTTCATC |
| | TCGTTGACTTTCAGGTTACTATAGCAGAGATATTACTAATTATTATGAGGACTTTTAAAGTTTCCAT |
| | TTGGAATCTTGATTACATCATAAACCTCATAATTAAAAATTTATCTAAGTCACTAACTGAGAATAAA |
| | TATTCTCAATTAGATGAAGAGCAACCAATGGAGATTGATTAAACGAACATGAAAATTATTCTTTTCT |
| | TGGCACTGATAACACTCGCTACTTGTGAGCTTTATCACTACCAAGAGTGTGTTAGAGGTACAACAGT |
| | ACTTTTAAAAGAACCTTGCTCTTCTGGAACATACGAGGGCAATTCACCATTTCATCCTCTAGCTGAT |
| | AACAAATTTGCACTGACTTGCTTTAGCACTCAATTTGCTTTTGCTTGTCCTGACGGCGTAAAACACG |
| | TCTATCAGTTACGTGCCAGATCAGTTTCACCTAAACTGTTCATCAGACAAGAGGAAGTTCAAGAACT |
| | TTACTCTCCAATTTTTCTTATTGTTGCGGCAATAGTGTTTATAACACTTTGCTTCACACTCAAAAGA |
| | AAGACAGAATGATTGAACTTTCATTAATTGACTTCTATTTGTGCTTTTTAGCCTTTCTGCTATTCCT |
| | TGTTTTAATTATGCTTATTATCTTTTGGTTCTCACTTGAACTGCAAGATCATAATGAAACTTGTCAC |
| | GCCTAAACGAACATGAAATTTCTTGTTTTCTTAGGAATCATCACAACTGTAGCTGCATTTCACCAAG |
| | AATGTAGTTTACAGTCATGTACTCAACATCAACCATATGTAGTTGATGACCCGTGTCCTATTCACTT |
| | CTATTCTAAATGGTATATTAGAGTAGGAGCTAGAAAATCAGCACCTTTAATTGAATTGTGCGTGGAT |
| | GAGGCTGGTTCTAAATCACCCATTCAGTACATCGATATCGGTAATTATACAGTTTCCTGTTTACCTT |
| | TTACAATTAATTGCCAGAAACCTAAATTGGGTAGTCTTGTAGTGCGTTGTTCGTTCTATGAAGACTT |
| | TTTAGAGTATCATGACGTTCGTGTTGTTTTAGATTTCATCTAAACGAACAAACAAACTAAAATGTCT |
| | GATAATGGACCCCAAAATCAGCGAAATGCACCCCGCATTACGTTTGGTGGACCCTCAGATTCAACTG |
| | GCAGTAACCAGAATGGAGAACGCAGTGGGGCGCGATCAAAACAACGTCGGCCCCAAGGTTTACCCAA |
| | TAATACTGCGTCTTGGTTCACCGCTCTCACTCAACATGGCAAGGAAGACCTTAAATTCCCTCGAGGA |
| | CAAGGCGTTCCAATTAACACCAATAGCAGTCGAGATGACCAAATTGGCTACTACCGAAGAGCTACCA |
| | GACGAATTCGTGGTGGTGACGGTAAAATGAAAGATCTCAGTCCAAGATGGTATTTCTACTACCTAGG |
| | AACTGGGCCAGAAGCTGGACTTCCCTATGGTGCTAACAAAGACGGCATCATATGGGTTGCAACTGAG |
| | GGAGCCTTGAATACACCAAAAGATCACATTGGCACCCGCAATCCTGCTAACAATGCTGCAATCGTGC |
| | TACAACTTCCTCAAGGAACAACATTGCCAAAAGGCTTCTACGCTGAAGGGAGCAGAGGCGGCAGTCA |
| | AGCCTCTTCTCGTTCCTCATCACGTAGTCGCAACAGTTCAAGAAATTCAACTCCAGGCAGCTCTAAA |
| | CGAACTTCTCCTGCTAGAATGGCTGGCAATGGCGGTGATGCTGCTCTTGCTTTGCTGCTGCTTGACA |
| | GATTGAACCAGCTTGAGAGCAAAATGTCTGGTAAAGGCCAACAACAACAAGGCCAAACTGTCACTAA |
| | GAAATCTGCTGCTGAGGCTTCTAAGAAGCCTCGGCAAAAACGTACTGCCACTAAAGCATACAATGTA |
| | ACACAAGCTTTCGGCAGACGTGGTCCAGAACAAACCCAAGGAAATTTTGGGGACCAGGAACTAATCA |
| | GACAAGGAACTGATTACAAACATTGGCCGCAAATTGCACAATTTGCCCCCAGCGCTTCAGCGTTCTT |
| | CGGAATGTCGCGCATTGGCATGGAAGTCACACCTTCGGGAACGTGGTTGACCTACACAGGTGCCATC |
| | AAATTGGATGACAAAGATCCAAATTTCAAAGATCAAGTCATTTTGCTGAATAAGCATATTGACGCAT |
| | ACAAAACATTCCCACCAACAGAGCCTAAAAAGGACAAAAAGAAGAAGGCTGATGAAACTCAAGCCTT |
| | ACCGCAGAGACAGAAGAAACAGCAAACTGTGACTCTTCTTCCTGCTGCAGATTTGGATGATTTCTCC |
| | AAACAATTGCAACAATCCATGAGCAGTGCTGACTCAACTCAGGCCTAAACTCATGCAGACCACACAA |
| | GGCAGATGGGCTATATAAACGTTTTCGCTTTTCCGTTTACGATATATAGTCTACTCTTGTGCAGAAT |
| | GAATTCTCGTAACTACATAGCACAAGTAGATGTAGTTAACTTTAATCTCACATAGCAATCTTTAATC |
| | AGTGTGTAACATTAGGGAGGACTTGAAAGAGCCACCACATTTTCACCGAGGCCACGCGGAGTACGAT |
| | CGAGTGTACAGTGAACAATGCTAGGGAGAGCTGCCTATATGGAAGAGCCCTAATGTGTAAAATTAAT |
| | TTTAGTAGTGCTAACCCCATGTGATTTTAATAGCTTCTTAGGAG |
| 279 | GenBank Accession No. MN985325 (Severe acute respiratory syndrome |
| | coronavirus 2 isolate SARS-CoV-2/human/USA/WA-CDC-WA1/2020, complete |
| | genome) |
| | ATTAAAGGTTTATACCTTCCCAGGTAACAAACCAACCAACTTTCGATCTCTTGTAGATCTGTTCTCT |
| | AAACGAACTTTAAAATCTGTGTGGCTGTCACTCGGCTGCATGCTTAGTGCACTCACGCAGTATAATT |
| | AATAACTAATTACTGTCGTTGACAGGACACGAGTAACTCGTCTATCTTCTGCAGGCTGCTTACGGTT |
| | TCGTCCGTGTTGCAGCCGATCATCAGCACATCTAGGTTTCGTCCGGGTGTGACCGAAAGGTAAGATG |
| | GAGAGCCTTGTCCCTGGTTTCAACGAGAAAACACACGTCCAACTCAGTTTGCCTGTTTTACAGGTTC |
| | GCGACGTGCTCGTACGTGGCTTTGGAGACTCCGTGGAGGAGGTCTTATCAGAGGCACGTCAACATCT |
| | TAAAGATGGCACTTGTGGCTTAGTAGAAGTTGAAAAAGGCGTTTTGCCTCAACTTGAACAGCCCTAT |
| | GTGTTCATCAAACGTTCGGATGCTCGAACTGCACCTCATGGTCATGTTATGGTTGAGCTGGTAGCAG |
| | AACTCGAAGGCATTCAGTACGGTCGTAGTGGTGAGACACTTGGTGTCCTTGTCCCTCATGTGGGCGA |
| | AATACCAGTGGCTTACCGCAAGGTTCTTCTTCGTAAGAACGGTAATAAAGGAGCTGGTGGCCATAGT |
| | TACGGCGCCGATCTAAAGTCATTTGACTTAGGCGACGAGCTTGGCACTGATCCTTATGAAGATTTTC |
| | AAGAAAACTGGAACACTAAACATAGCAGTGGTGTTACCCGTGAACTCATGCGTGAGCTTAACGGAGG |
| | GGCATACACTCGCTATGTCGATAACAACTTCTGTGGCCCTGATGGCTACCCTCTTGAGTGCATTAAA |
| | GACCTTCTAGCACGTGCTGGTAAAGCTTCATGCACTTTGTCCGAACAACTGGACTTTATTGACACTA |
| | AGAGGGGTGTATACTGCTGCCGTGAACATGAGCATGAAATTGCTTGGTACACGGAACGTTCTGAAAA |
| | GAGCTATGAATTGCAGACACCTTTTGAAATTAAATTGGCAAAGAAATTTGACACCTTCAATGGGGAA |
| | TGTCCAAATTTTGTATTTCCCTTAAATTCCATAATCAAGACTATTCAACCAAGGGTTGAAAAGAAAA |
| | AGCTTGATGGCTTTATGGGTAGAATTCGATCTGTCTATCCAGTTGCGTCACCAAATGAATGCAACCA |
| | AATGTGCCTTTCAACTCTCATGAAGTGTGATCATTGTGGTGAAACTTCATGGCAGACGGGCGATTTT |
| | GTTAAAGCCACTTGCGAATTTTGTGGCACTGAGAATTTGACTAAAGAAGGTGCCACTACTTGTGGTT |
| | ACTTACCCCAAAATGCTGTTGTTAAAATTTATTGTCCAGCATGTCACAATTCAGAAGTAGGACCTGA |
| | GCATAGTCTTGCCGAATACCATAATGAATCTGGCTTGAAAACCATTCTTCGTAAGGGTGGTCGCACT |
| | ATTGCCTTTGGAGGCTGTGTGTTCTCTTATGTTGGTTGCCATAACAAGTGTGCCTATTGGGTTCCAC |
| | GTGCTAGCGCTAACATAGGTTGTAACCATACAGGTGTTGTTGGAGAAGGTTCCGAAGGTCTTAATGA |
| | CAACCTTCTTGAAATACTCCAAAAAGAGAAAGTCAACATCAATATTGTTGGTGACTTTAAACTTAAT |
| | GAAGAGATCGCCATTATTTTGGCATCTTTTTCTGCTTCCACAAGTGCTTTTGTGGAAACTGTGAAAG |
| | GTTTGGATTATAAAGCATTCAAACAAATTGTTGAATCCTGTGGTAATTTTAAAGTTACAAAAGGAAA |
| | AGCTAAAAAAGGTGCCTGGAATATTGGTGAACAGAAATCAATACTGAGTCCTCTTTATGCATTTGCA |
| | TCAGAGGCTGCTCGTGTTGTACGATCAATTTTCTCCCGCACTCTTGAAACTGCTCAAAATTCTGTGC |
| | GTGTTTTACAGAAGGCCGCTATAACAATACTAGATGGAATTTCACAGTATTCACTGAGACTCATTGA |
| | TGCTATGATGTTCACATCTGATTTGGCTACTAACAATCTAGTTGTAATGGCCTACATTACAGGTGGT |
| | GTTGTTCAGTTGACTTCGCAGTGGCTAACTAACATCTTTGGCACTGTTTATGAAAAACTCAAACCCG |
| | TCCTTGATTGGCTTGAAGAGAAGTTTAAGGAAGGTGTAGAGTTTCTTAGAGACGGTTGGGAAATTGT |
| | TAAATTTATCTCAACCTGTGCTTGTGAAATTGTCGGTGGACAAATTGTCACCTGTGCAAAGGAAATT |
| | AAGGAGAGTGTTCAGACATTCTTTAAGCTTGTAAATAAATTTTTGGCTTTGTGTGCTGACTCTATCA |
| | TTATTGGTGGAGCTAAACTTAAAGCCTTGAATTTAGGTGAAACATTTGTCACGCACTCAAAGGGATT |
| | GTACAGAAAGTGTGTTAAATCCAGAGAAGAAACTGGCCTACTCATGCCTCTAAAAGCCCCAAAAGAA |
| | ATTATCTTCTTAGAGGGAGAAACACTTCCCACAGAAGTGTTAACAGAGGAAGTTGTCTTGAAAACTG |
| | GTGATTTACAACCATTAGAACAACCTACTAGTGAAGCTGTTGAAGCTCCATTGGTTGGTACACCAGT |
| | TTGTATTAACGGGCTTATGTTGCTCGAAATCAAAGACACAGAAAAGTACTGTGCCCTTGCACCTAAT |
| | ATGATGGTAACAAACAATACCTTCACACTCAAAGGCGGTGCACCAACAAAGGTTACTTTTGGTGATG |
| | ACACTGTGATAGAAGTGCAAGGTTACAAGAGTGTGAATATCACTTTTGAACTTGATGAAAGGATTGA |
| | TAAAGTACTTAATGAGAAGTGCTCTGCCTATACAGTTGAACTCGGTACAGAAGTAAATGAGTTCGCC |
| | TGTGTTGTGGCAGATGCTGTCATAAAAACTTTGCAACCAGTATCTGAATTACTTACACCACTGGGCA |
| | TTGATTTAGATGAGTGGAGTATGGCTACATACTACTTATTTGATGAGTCTGGTGAGTTTAAATTGGC |
| | TTCACATATGTATTGTTCTTTCTACCCTCCAGATGAGGATGAAGAAGAAGGTGATTGTGAAGAAGAA |
| | GAGTTTGAGCCATCAACTCAATATGAGTATGGTACTGAAGATGATTACCAAGGTAAACCTTTGGAAT |
| | TTGGTGCCACTTCTGCTGCTCTTCAACCTGAAGAAGAGCAAGAAGAAGATTGGTTAGATGATGATAG |
| | TCAACAAACTGTTGGTCAACAAGACGGCAGTGAGGACAATCAGACAACTACTATTCAAACAATTGTT |
| | GAGGTTCAACCTCAATTAGAGATGGAACTTACACCAGTTGTTCAGACTATTGAAGTGAATAGTTTTA |
| | GTGGTTATTTAAAACTTACTGACAATGTATACATTAAAAATGCAGACATTGTGGAAGAAGCTAAAAA |
| | GGTAAAACCAACAGTGGTTGTTAATGCAGCCAATGTTTACCTTAAACATGGAGGAGGTGTTGCAGGA |
| | GCCTTAAATAAGGCTACTAACAATGCCATGCAAGTTGAATCTGATGATTACATAGCTACTAATGGAC |
| | CACTTAAAGTGGGTGGTAGTTGTGTTTTAAGCGGACACAATCTTGCTAAACACTGTCTTCATGTTGT |
| | CGGCCCAAATGTTAACAAAGGTGAAGACATTCAACTTCTTAAGAGTGCTTATGAAAATTTTAATCAG |
| | CACGAAGTTCTACTTGCACCATTATTATCAGCTGGTATTTTTGGTGCTGACCCTATACATTCTTTAA |
| | GAGTTTGTGTAGATACTGTTCGCACAAATGTCTACTTAGCTGTCTTTGATAAAAATCTCTATGACAA |
| | ACTTGTTTCAAGCTTTTTGGAAATGAAGAGTGAAAAGCAAGTTGAACAAAAGATCGCTGAGATTCCT |
| | AAAGAGGAAGTTAAGCCATTTATAACTGAAAGTAAACCTTCAGTTGAACAGAGAAAACAAGATGATA |
| | AGAAAATCAAAGCTTGTGTTGAAGAAGTTACAACAACTCTGGAAGAAACTAAGTTCCTCACAGAAAA |
| | CTTGTTACTTTATATTGACATTAATGGCAATCTTCATCCAGATTCTGCCACTCTTGTTAGTGACATT |
| | GACATCACTTTCTTAAAGAAAGATGCTCCATATATAGTGGGTGATGTTGTTCAAGAGGGTGTTTTAA |
| | CTGCTGTGGTTATACCTACTAAAAAGGCTGGTGGCACTACTGAAATGCTAGCGAAAGCTTTGAGAAA |
| | AGTGCCAACAGACAATTATATAACCACTTACCCGGGTCAGGGTTTAAATGGTTACACTGTAGAGGAG |
| | GCAAAGACAGTGCTTAAAAAGTGTAAAAGTGCCTTTTACATTCTACCATCTATTATCTCTAATGAGA |
| | AGCAAGAAATTCTTGGAACTGTTTCTTGGAATTTGCGAGAAATGCTTGCACATGCAGAAGAAACACG |
| | CAAATTAATGCCTGTCTGTGTGGAAACTAAAGCCATAGTTTCAACTATACAGCGTAAATATAAGGGT |
| | ATTAAAATACAAGAGGGTGTGGTTGATTATGGTGCTAGATTTTACTTTTACACCAGTAAAACAACTG |
| | TAGCGTCACTTATCAACACACTTAACGATCTAAATGAAACTCTTGTTACAATGCCACTTGGCTATGT |
| | AACACATGGCTTAAATTTGGAAGAAGCTGCTCGGTATATGAGATCTCTCAAAGTGCCAGCTACAGTT |
| | TCTGTTTCTTCACCTGATGCTGTTACAGCGTATAATGGTTATCTTACTTCTTCTTCTAAAACACCTG |
| | AAGAACATTTTATTGAAACCATCTCACTTGCTGGTTCCTATAAAGATTGGTCCTATTCTGGACAATC |
| | TACACAACTAGGTATAGAATTTCTTAAGAGAGGTGATAAAAGTGTATATTACACTAGTAATCCTACC |
| | ACATTCCACCTAGATGGTGAAGTTATCACCTTTGACAATCTTAAGACACTTCTTTCTTTGAGAGAAG |
| | TGAGGACTATTAAGGTGTTTACAACAGTAGACAACATTAACCTCCACACGCAAGTTGTGGACATGTC |
| | AATGACATATGGACAACAGTTTGGTCCAACTTATTTGGATGGAGCTGATGTTACTAAAATAAAACCT |
| | CATAATTCACATGAAGGTAAAACATTTTATGTTTTACCTAATGATGACACTCTACGTGTTGAGGCTT |
| | TTGAGTACTACCACACAACTGATCCTAGTTTTCTGGGTAGGTACATGTCAGCATTAAATCACACTAA |
| | AAAGTGGAAATACCCACAAGTTAATGGTTTAACTTCTATTAAATGGGCAGATAACAACTGTTATCTT |
| | GCCACTGCATTGTTAACACTCCAACAAATAGAGTTGAAGTTTAATCCACCTGCTCTACAAGATGCTT |
| | ATTACAGAGCAAGGGCTGGTGAAGCTGCTAACTTTTGTGCACTTATCTTAGCCTACTGTAATAAGAC |
| | AGTAGGTGAGTTAGGTGATGTTAGAGAAACAATGAGTTACTTGTTTCAACATGCCAATTTAGATTCT |
| | TGCAAAAGAGTCTTGAACGTGGTGTGTAAAACTTGTGGACAACAGCAGACAACCCTTAAGGGTGTAG |
| | AAGCTGTTATGTACATGGGCACACTTTCTTATGAACAATTTAAGAAAGGTGTTCAGATACCTTGTAC |
| | GTGTGGTAAACAAGCTACAAAATATCTAGTACAACAGGAGTCACCTTTTGTTATGATGTCAGCACCA |
| | CCTGCTCAGTATGAACTTAAGCATGGTACATTTACTTGTGCTAGTGAGTACACTGGTAATTACCAGT |
| | GTGGTCACTATAAACATATAACTTCTAAAGAAACTTTGTATTGCATAGACGGTGCTTTACTTACAAA |
| | GTCCTCAGAATACAAAGGTCCTATTACGGATGTTTTCTACAAAGAAAACAGTTACACAACAACCATA |
| | AAACCAGTTACTTATAAATTGGATGGTGTTGTTTGTACAGAAATTGACCCTAAGTTGGACAATTATT |
| | ATAAGAAAGACAATTCTTATTTCACAGAGCAACCAATTGATCTTGTACCAAACCAACCATATCCAAA |
| | CGCAAGCTTCGATAATTTTAAGTTTGTATGTGATAATATCAAATTTGCTGATGATTTAAACCAGTTA |
| | ACTGGTTATAAGAAACCTGCTTCAAGAGAGCTTAAAGTTACATTTTTCCCTGACTTAAATGGTGATG |
| | TGGTGGCTATTGATTATAAACACTACACACCCTCTTTTAAGAAAGGAGCTAAATTGTTACATAAACC |
| | TATTGTTTGGCATGTTAACAATGCAACTAATAAAGCCACGTATAAACCAAATACCTGGTGTATACGT |
| | TGTCTTTGGAGCACAAAACCAGTTGAAACATCAAATTCGTTTGATGTACTGAAGTCAGAGGACGCGC |
| | AGGGAATGGATAATCTTGCCTGCGAAGATCTAAAACCAGTCTCTGAAGAAGTAGTGGAAAATCCTAC |
| | CATACAGAAAGACGTTCTTGAGTGTAATGTGAAAACTACCGAAGTTGTAGGAGACATTATACTTAAA |
| | CCAGCAAATAATAGTTTAAAAATTACAGAAGAGGTTGGCCACACAGATCTAATGGCTGCTTATGTAG |
| | ACAATTCTAGTCTTACTATTAAGAAACCTAATGAATTATCTAGAGTATTAGGTTTGAAAACCCTTGC |
| | TACTCATGGTTTAGCTGCTGTTAATAGTGTCCCTTGGGATACTATAGCTAATTATGCTAAGCCTTTT |
| | CTTAACAAAGTTGTTAGTACAACTACTAACATAGTTACACGGTGTTTAAACCGTGTTTGTACTAATT |
| | ATATGCCTTATTTCTTTACTTTATTGCTACAATTGTGTACTTTTACTAGAAGTACAAATTCTAGAAT |
| | TAAAGCATCTATGCCGACTACTATAGCAAAGAATACTGTTAAGAGTGTCGGTAAATTTTGTCTAGAG |
| | GCTTCATTTAATTATTTGAAGTCACCTAATTTTTCTAAACTGATAAATATTATAATTTGGTTTTTAC |
| | TATTAAGTGTTTGCCTAGGTTCTTTAATCTACTCAACCGCTGCTTTAGGTGTTTTAATGTCTAATTT |
| | AGGCATGCCTTCTTACTGTACTGGTTACAGAGAAGGCTATTTGAACTCTACTAATGTCACTATTGCA |
| | ACCTACTGTACTGGTTCTATACCTTGTAGTGTTTGTCTTAGTGGTTTAGATTCTTTAGACACCTATC |
| | CTTCTTTAGAAACTATACAAATTACCATTTCATCTTTTAAATGGGATTTAACTGCTTTTGGCTTAGT |
| | TGCAGAGTGGTTTTTGGCATATATTCTTTTCACTAGGTTTTTCTATGTACTTGGATTGGCTGCAATC |
| | ATGCAATTGTTTTTCAGCTATTTTGCAGTACATTTTATTAGTAATTCTTGGCTTATGTGGTTAATAA |
| | TTAATCTTGTACAAATGGCCCCGATTTCAGCTATGGTTAGAATGTACATCTTCTTTGCATCATTTTA |
| | TTATGTATGGAAAAGTTATGTGCATGTTGTAGACGGTTGTAATTCATCAACTTGTATGATGTGTTAC |
| | AAACGTAATAGAGCAACAAGAGTCGAATGTACAACTATTGTTAATGGTGTTAGAAGGTCCTTTTATG |
| | TCTATGCTAATGGAGGTAAAGGCTTTTGCAAACTACACAATTGGAATTGTGTTAATTGTGATACATT |
| | CTGTGCTGGTAGTACATTTATTAGTGATGAAGTTGCGAGAGACTTGTCACTACAGTTTAAAAGACCA |
| | ATAAATCCTACTGACCAGTCTTCTTACATCGTTGATAGTGTTACAGTGAAGAATGGTTCCATCCATC |
| | TTTACTTTGATAAAGCTGGTCAAAAGACTTATGAAAGACATTCTCTCTCTCATTTTGTTAACTTAGA |
| | CAACCTGAGAGCTAATAACACTAAAGGTTCATTGCCTATTAATGTTATAGTTTTTGATGGTAAATCA |
| | AAATGTGAAGAATCATCTGCAAAATCAGCGTCTGTTTACTACAGTCAGCTTATGTGTCAACCTATAC |
| | TGTTACTAGATCAGGCATTAGTGTCTGATGTTGGTGATAGTGCGGAAGTTGCAGTTAAAATGTTTGA |
| | TGCTTACGTTAATACGTTTTCATCAACTTTTAACGTACCAATGGAAAAACTCAAAACACTAGTTGCA |
| | ACTGCAGAAGCTGAACTTGCAAAGAATGTGTCCTTAGACAATGTCTTATCTACTTTTATTTCAGCAG |
| | CTCGGCAAGGGTTTGTTGATTCAGATGTAGAAACTAAAGATGTTGTTGAATGTCTTAAATTGTCACA |
| | TCAATCTGACATAGAAGTTACTGGCGATAGTTGTAATAACTATATGCTCACCTATAACAAAGTTGAA |
| | AACATGACACCCCGTGACCTTGGTGCTTGTATTGACTGTAGTGCGCGTCATATTAATGCGCAGGTAG |
| | CAAAAAGTCACAACATTGCTTTGATATGGAACGTTAAAGATTTCATGTCATTGTCTGAACAACTACG |
| | AAAACAAATACGTAGTGCTGCTAAAAAGAATAACTTACCTTTTAAGTTGACATGTGCAACTACTAGA |
| | CAAGTTGTTAATGTTGTAACAACAAAGATAGCACTTAAGGGTGGTAAAATTGTTAATAATTGGTTGA |
| | AGCAGTTAATTAAAGTTACACTTGTGTTCCTTTTTGTTGCTGCTATTTTCTATTTAATAACACCTGT |
| | TCATGTCATGTCTAAACATACTGACTTTTCAAGTGAAATCATAGGATACAAGGCTATTGATGGTGGT |
| | GTCACTCGTGACATAGCATCTACAGATACTTGTTTTGCTAACAAACATGCTGATTTTGACACATGGT |
| | TTAGTCAGCGTGGTGGTAGTTATACTAATGACAAAGCTTGCCCATTGATTGCTGCAGTCATAACAAG |
| | AGAAGTGGGTTTTGTCGTGCCTGGTTTGCCTGGCACGATATTACGCACAACTAATGGTGACTTTTTG |
| | CATTTCTTACCTAGAGTTTTTAGTGCAGTTGGTAACATCTGTTACACACCATCAAAACTTATAGAGT |
| | ACACTGACTTTGCAACATCAGCTTGTGTTTTGGCTGCTGAATGTACAATTTTTAAAGATGCTTCTGG |
| | TAAGCCAGTACCATATTGTTATGATACCAATGTACTAGAAGGTTCTGTTGCTTATGAAAGTTTACGC |
| | CCTGACACACGTTATGTGCTCATGGATGGCTCTATTATTCAATTTCCTAACACCTACCTTGAAGGTT |
| | CTGTTAGAGTGGTAACAACTTTTGATTCTGAGTACTGTAGGCACGGCACTTGTGAAAGATCAGAAGC |
| | TGGTGTTTGTGTATCTACTAGTGGTAGATGGGTACTTAACAATGATTATTACAGATCTTTACCAGGA |
| | GTTTTCTGTGGTGTAGATGCTGTAAATTTACTTACTAATATGTTTACACCACTAATTCAACCTATTG |
| | GTGCTTTGGACATATCAGCATCTATAGTAGCTGGTGGTATTGTAGCTATCGTAGTAACATGCCTTGC |
| | CTACTATTTTATGAGGTTTAGAAGAGCTTTTGGTGAATACAGTCATGTAGTTGCCTTTAATACTTTA |
| | CTATTCCTTATGTCATTCACTGTACTCTGTTTAACACCAGTTTACTCATTCTTACCTGGTGTTTATT |
| | CTGTTATTTACTTGTACTTGACATTTTATCTTACTAATGATGTTTCTTTTTTAGCACATATTCAGTG |
| | GATGGTTATGTTCACACCTTTAGTACCTTTCTGGATAACAATTGCTTATATCATTTGTATTTCCACA |
| | AAGCATTTCTATTGGTTCTTTAGTAATTACCTAAAGAGACGTGTAGTCTTTAATGGTGTTTCCTTTA |
| | GTACTTTTGAAGAAGCTGCGCTGTGCACCTTTTTGTTAAATAAAGAAATGTATCTAAAGTTGCGTAG |
| | TGATGTGCTATTACCTCTTACGCAATATAATAGATACTTAGCTCTTTATAATAAGTACAAGTATTTT |
| | AGTGGAGCAATGGATACAACTAGCTACAGAGAAGCTGCTTGTTGTCATCTCGCAAAGGCTCTCAATG |
| | ACTTCAGTAACTCAGGTTCTGATGTTCTTTACCAACCACCACAAACCTCTATCACCTCAGCTGTTTT |
| | GCAGAGTGGTTTTAGAAAAATGGCATTCCCATCTGGTAAAGTTGAGGGTTGTATGGTACAAGTAACT |
| | TGTGGTACAACTACACTTAACGGTCTTTGGCTTGATGACGTAGTTTACTGTCCAAGACATGTGATCT |
| | GCACCTCTGAAGACATGCTTAACCCTAATTATGAAGATTTACTCATTCGTAAGTCTAATCATAATTT |
| | CTTGGTACAGGCTGGTAATGTTCAACTCAGGGTTATTGGACATTCTATGCAAAATTGTGTACTTAAG |
| | CTTAAGGTTGATACAGCCAATCCTAAGACACCTAAGTATAAGTTTGTTCGCATTCAACCAGGACAGA |
| | CTTTTTCAGTGTTAGCTTGTTACAATGGTTCACCATCTGGTGTTTACCAATGTGCTATGAGGCCCAA |
| | TTTCACTATTAAGGGTTCATTCCTTAATGGTTCATGTGGTAGTGTTGGTTTTAACATAGATTATGAC |
| | TGTGTCTCTTTTTGTTACATGCACCATATGGAATTACCAACTGGAGTTCATGCTGGCACAGACTTAG |
| | AAGGTAACTTTTATGGACCTTTTGTTGACAGGCAAACAGCACAAGCAGCTGGTACGGACACAACTAT |
| | TACAGTTAATGTTTTAGCTTGGTTGTACGCTGCTGTTATAAATGGAGACAGGTGGTTTCTCAATCGA |
| | TTTACCACAACTCTTAATGACTTTAACCTTGTGGCTATGAAGTACAATTATGAACCTCTAACACAAG |
| | ACCATGTTGACATACTAGGACCTCTTTCTGCTCAAACTGGAATTGCCGTTTTAGATATGTGTGCTTC |
| | ATTAAAAGAATTACTGCAAAATGGTATGAATGGACGTACCATATTGGGTAGTGCTTTATTAGAAGAT |
| | GAATTTACACCTTTTGATGTTGTTAGACAATGCTCAGGTGTTACTTTCCAAAGTGCAGTGAAAAGAA |
| | CAATCAAGGGTACACACCACTGGTTGTTACTCACAATTTTGACTTCACTTTTAGTTTTAGTCCAGAG |
| | TACTCAATGGTCTTTGTTCTTTTTTTTGTATGAAAATGCCTTTTTACCTTTTGCTATGGGTATTATT |
| | GCTATGTCTGCTTTTGCAATGATGTTTGTCAAACATAAGCATGCATTTCTCTGTTTGTTTTTGTTAC |
| | CTTCTCTTGCCACTGTAGCTTATTTTAATATGGTCTATATGCCTGCTAGTTGGGTGATGCGTATTAT |
| | GACATGGTTGGATATGGTTGATACTAGTTTGTCTGGTTTTAAGCTAAAAGACTGTGTTATGTATGCA |
| | TCAGCTGTAGTGTTACTAATCCTTATGACAGCAAGAACTGTGTATGATGATGGTGCTAGGAGAGTGT |
| | GGACACTTATGAATGTCTTGACACTCGTTTATAAAGTTTATTATGGTAATGCTTTAGATCAAGCCAT |
| | TTCCATGTGGGCTCTTATAATCTCTGTTACTTCTAACTACTCAGGTGTAGTTACAACTGTCATGTTT |
| | TTGGCCAGAGGTATTGTTTTTATGTGTGTTGAGTATTGCCCTATTTTCTTCATAACTGGTAATACAC |
| | TTCAGTGTATAATGCTAGTTTATTGTTTCTTAGGCTATTTTTGTACTTGTTACTTTGGCCTCTTTTG |
| | TTTACTCAACCGCTACTTTAGACTGACTCTTGGTGTTTATGATTACTTAGTTTCTACACAGGAGTTT |
| | AGATATATGAATTCACAGGGACTACTCCCACCCAAGAATAGCATAGATGCCTTCAAACTCAACATTA |
| | AATTGTTGGGTGTTGGTGGCAAACCTTGTATCAAAGTAGCCACTGTACAGTCTAAAATGTCAGATGT |
| | AAAGTGCACATCAGTAGTCTTACTCTCAGTTTTGCAACAACTCAGAGTAGAATCATCATCTAAATTG |
| | TGGGCTCAATGTGTCCAGTTACACAATGACATTCTCTTAGCTAAAGATACTACTGAAGCCTTTGAAA |
| | AAATGGTTTCACTACTTTCTGTTTTGCTTTCCATGCAGGGTGCTGTAGACATAAACAAGCTTTGTGA |
| | AGAAATGCTGGACAACAGGGCAACCTTACAAGCTATAGCCTCAGAGTTTAGTTCCCTTCCATCATAT |
| | GCAGCTTTTGCTACTGCTCAAGAAGCTTATGAGCAGGCTGTTGCTAATGGTGATTCTGAAGTTGTTC |
| | TTAAAAAGTTGAAGAAGTCTTTGAATGTGGCTAAATCTGAATTTGACCGTGATGCAGCCATGCAACG |
| | TAAGTTGGAAAAGATGGCTGATCAAGCTATGACCCAAATGTATAAACAGGCTAGATCTGAGGACAAG |
| | AGGGCAAAAGTTACTAGTGCTATGCAGACAATGCTTTTCACTATGCTTAGAAAGTTGGATAATGATG |
| | CACTCAACAACATTATCAACAATGCAAGAGATGGTTGTGTTCCCTTGAACATAATACCTCTTACAAC |
| | AGCAGCCAAACTAATGGTTGTCATACCAGACTATAACACATATAAAAATACGTGTGATGGTACAACA |
| | TTTACTTATGCATCAGCATTGTGGGAAATCCAACAGGTTGTAGATGCAGATAGTAAAATTGTTCAAC |
| | TTAGTGAAATTAGTATGGACAATTCACCTAATTTAGCATGGCCTCTTATTGTAACAGCTTTAAGGGC |
| | CAATTCTGCTGTCAAATTACAGAATAATGAGCTTAGTCCTGTTGCACTACGACAGATGTCTTGTGCT |
| | GCCGGTACTACACAAACTGCTTGCACTGATGACAATGCGTTAGCTTACTACAACACAACAAAGGGAG |
| | GTAGGTTTGTACTTGCACTGTTATCCGATTTACAGGATTTGAAATGGGCTAGATTCCCTAAGAGTGA |
| | TGGAACTGGTACTATCTATACAGAACTGGAACCACCTTGTAGGTTTGTTACAGACACACCTAAAGGT |
| | CCTAAAGTGAAGTATTTATACTTTATTAAAGGATTAAACAACCTAAATAGAGGTATGGTACTTGGTA |
| | GTTTAGCTGCCACAGTACGTCTACAAGCTGGTAATGCAACAGAAGTGCCTGCCAATTCAACTGTATT |
| | ATCTTTCTGTGCTTTTGCTGTAGATGCTGCTAAAGCTTACAAAGATTATCTAGCTAGTGGGGGACAA |
| | CCAATCACTAATTGTGTTAAGATGTTGTGTACACACACTGGTACTGGTCAGGCAATAACAGTTACAC |
| | CGGAAGCCAATATGGATCAAGAATCCTTTGGTGGTGCATCGTGTTGTCTGTACTGCCGTTGCCACAT |
| | AGATCATCCAAATCCTAAAGGATTTTGTGACTTAAAAGGTAAGTATGTACAAATACCTACAACTTGT |
| | GCTAATGACCCTGTGGGTTTTACACTTAAAAACACAGTCTGTACCGTCTGCGGTATGTGGAAAGGTT |
| | ATGGCTGTAGTTGTGATCAACTCCGCGAACCCATGCTTCAGTCAGCTGATGCACAATCGTTTTTAAA |
| | CGGGTTTGCGGTGTAAGTGCAGCCCGTCTTACACCGTGCGGCACAGGCACTAGTACTGATGTCGTAT |
| | ACAGGGCTTTTGACATCTACAATGATAAAGTAGCTGGTTTTGCTAAATTCCTAAAAACTAATTGTTG |
| | TCGCTTCCAAGAAAAGGACGAAGATGACAATTTAATTGATTCTTACTTTGTAGTTAAGAGACACACT |
| | TTCTCTAACTACCAACATGAAGAAACAATTTATAATTTACTTAAGGATTGTCCAGCTGTTGCTAAAC |
| | ATGACTTCTTTAAGTTTAGAATAGACGGTGACATGGTACCACATATATCACGTCAACGTCTTACTAA |
| | ATACACAATGGCAGACCTCGTCTATGCTTTAAGGCATTTTGATGAAGGTAATTGTGACACATTAAAA |
| | GAAATACTTGTCACATACAATTGTTGTGATGATGATTATTTCAATAAAAAGGACTGGTATGATTTTG |
| | TAGAAAACCCAGATATATTACGCGTATACGCCAACTTAGGTGAACGTGTACGCCAAGCTTTGTTAAA |
| | AACAGTACAATTCTGTGATGCCATGCGAAATGCTGGTATTGTTGGTGTACTGACATTAGATAATCAA |
| | GATCTCAATGGTAACTGGTATGATTTCGGTGATTTCATACAAACCACGCCAGGTAGTGGAGTTCCTG |
| | TTGTAGATTCTTATTATTCATTGTTAATGCCTATATTAACCTTGACCAGGGCTTTAACTGCAGAGTC |
| | ACATGTTGACACTGACTTAACAAAGCCTTACATTAAGTGGGATTTGTTAAAATATGACTTCACGGAA |
| | GAGAGGTTAAAACTCTTTGACCGTTATTTTAAATATTGGGATCAGACATACCACCCAAATTGTGTTA |
| | ACTGTTTGGATGACAGATGCATTCTGCATTGTGCAAACTTTAATGTTTTATTCTCTACAGTGTTCCC |
| | ACCTACAAGTTTTGGACCACTAGTGAGAAAAATATTTGTTGATGGTGTTCCATTTGTAGTTTCAACT |
| | GGATACCACTTCAGAGAGCTAGGTGTTGTACATAATCAGGATGTAAACTTACATAGCTCTAGACTTA |
| | GTTTTAAGGAATTACTTGTGTATGCTGCTGACCCTGCTATGCACGCTGCTTCTGGTAATCTATTACT |
| | AGATAAACGCACTACGTGCTTTTCAGTAGCTGCACTTACTAACAATGTTGCTTTTCAAACTGTCAAA |
| | CCCGGTAATTTTAACAAAGACTTCTATGACTTTGCTGTGTCTAAGGGTTTCTTTAAGGAAGGAAGTT |
| | CTGTTGAATTAAAACACTTCTTCTTTGCTCAGGATGGTAATGCTGCTATCAGCGATTATGACTACTA |
| | TCGTTATAATCTACCAACAATGTGTGATATCAGACAACTACTATTTGTAGTTGAAGTTGTTGATAAG |
| | TACTTTGATTGTTACGATGGTGGCTGTATTAATGCTAACCAAGTCATCGTCAACAACCTAGACAAAT |
| | CAGCTGGTTTTCCATTTAATAAATGGGGTAAGGCTAGACTTTATTATGATTCAATGAGTTATGAGGA |
| | TCAAGATGCACTTTTCGCATATACAAAACGTAATGTCATCCCTACTATAACTCAAATGAATCTTAAG |
| | TATGCCATTAGTGCAAAGAATAGAGCTCGCACCGTAGCTGGTGTCTCTATCTGTAGTACTATGACCA |
| | ATAGACAGTTTCATCAAAAATTATTGAAATCAATAGCCGCCACTAGAGGAGCTACTGTAGTAATTGG |
| | AACAAGCAAATTCTATGGTGGTTGGCACAACATGTTAAAAACTGTTTATAGTGATGTAGAAAACCCT |
| | CACCTTATGGGTTGGGATTATCCTAAATGTGATAGAGCCATGCCTAACATGCTTAGAATTATGGCCT |
| | CACTTGTTCTTGCTCGCAAACATACAACGTGTTGTAGCTTGTCACACCGTTTCTATAGATTAGCTAA |
| | TGAGTGTGCTCAAGTATTGAGTGAAATGGTCATGTGTGGCGGTTCACTATATGTTAAACCAGGTGGA |
| | ACCTCATCAGGAGATGCCACAACTGCTTATGCTAATAGTGTTTTTAACATTTGTCAAGCTGTCACGG |
| | CCAATGTTAATGCACTTTTATCTACTGATGGTAACAAAATTGCCGATAAGTATGTCCGCAATTTACA |
| | ACACAGACTTTATGAGTGTCTCTATAGAAATAGAGATGTTGACACAGACTTTGTGAATGAGTTTTAG |
| | GCATATTTGCGTAAACATTTCTCAATGATGATACTCTCTGACGATGCTGTTGTGTGTTTCAATAGCA |
| | CTTATGCATCTCAAGGTCTAGTGGCTAGCATAAAGAACTTTAAGTCAGTTCTTTATTATCAAAACAA |
| | TGTTTTTATGTCTGAAGCAAAATGTTGGACTGAGACTGACCTTACTAAAGGACCTCATGAATTTTGC |
| | TCTCAACATACAATGCTAGTTAAACAGGGTGATGATTATGTGTACCTTCCTTACCCAGATCCATCAA |
| | GAATCCTAGGGGCCGGCTGTTTTGTAGATGATATCGTAAAAACAGATGGTACACTTATGATTGAACG |
| | GTTCGTGTCTTTAGCTATAGATGCTTACCCACTTACTAAACATCCTAATCAGGAGTATGCTGATGTC |
| | TTTCATTTGTACTTACAATACATAAGAAAGCTAGATGATGAGTTAACAGGACACATGTTAGACATGT |
| | ATTCTGTTATGCTTACTAATGATAACACTTCAAGGTATTGGGAACCTGAGTTTTATGAGGCTATGTA |
| | CACACCGCATACAGTCTTACAGGCTGTTGGGGCTTGTGTTCTTTGCAATTCACAGACTTCATTAAGA |
| | TGTGGTGCTTGCATACGTAGACCATTCTTATGTTGTAAATGCTGTTACGACCATGTCATATCAACAT |
| | CACATAAATTAGTCTTGTCTGTTAATCCGTATGTTTGCAATGCTCCAGGTTGTGATGTCACAGATGT |
| | GACTCAACTTTACTTAGGAGGTATGAGCTATTATTGTAAATCACATAAACCACCCATTAGTTTTCCA |
| | TTGTGTGCTAATGGACAAGTTTTTGGTTTATATAAAAATACATGTGTTGGTAGCGATAATGTTACTG |
| | ACTTTAATGCAATTGCAACATGTGACTGGACAAATGCTGGTGATTACATTTTAGCTAACACCTGTAC |
| | TGAAAGACTCAAGCTTTTTGCAGCAGAAACGCTCAAAGCTACTGAGGAGACATTTAAACTGTCTTAT |
| | GGTATTGCTACTGTACGTGAAGTGCTGTCTGACAGAGAATTACATCTTTCATGGGAAGTTGGTAAAC |
| | CTAGACCACCACTTAACCGAAATTATGTCTTTACTGGTTATCGTGTAACTAAAAACAGTAAAGTACA |
| | AATAGGAGAGTACACCTTTGAAAAAGGTGACTATGGTGATGCTGTTGTTTACCGAGGTACAACAACT |
| | TACAAATTAAATGTTGGTGATTATTTTGTGCTGACATCACATACAGTAATGCCATTAAGTGCACCTA |
| | CACTAGTGCCACAAGAGCACTATGTTAGAATTACTGGCTTATACCCAACACTCAATATCTCAGATGA |
| | GTTTTCTAGCAATGTTGCAAATTATCAAAAGGTTGGTATGCAAAAGTATTCTACACTCCAGGGACCA |
| | CCTGGTACTGGTAAGAGTCATTTTGCTATTGGCCTAGCTCTCTACTACCCTTCTGCTCGCATAGTGT |
| | ATACAGCTTGCTCTCATGCCGCTGTTGATGCACTATGTGAGAAGGCATTAAAATATTTGCCTATAGA |
| | TAAATGTAGTAGAATTATACCTGCACGTGCTCGTGTAGAGTGTTTTGATAAATTCAAAGTGAATTCA |
| | ACATTAGAACAGTATGTCTTTTGTACTGTAAATGCATTGCCTGAGACGACAGCAGATATAGTTGTCT |
| | TTGATGAAATTTCAATGGCCACAAATTATGATTTGAGTGTTGTCAATGCCAGATTACGTGCTAAGCA |
| | CTATGTGTACATTGGCGACCCTGCTCAATTACCTGCACCACGCACATTGCTAACTAAGGGCACACTA |
| | GAACCAGAATATTTCAATTCAGTGTGTAGACTTATGAAAACTATAGGTCCAGACATGTTCCTCGGAA |
| | CTTGTCGGCGTTGTCCTGCTGAAATTGTTGACACTGTGAGTGCTTTGGTTTATGATAATAAGCTTAA |
| | AGCACATAAAGACAAATCAGCTCAATGCTTTAAAATGTTTTATAAGGGTGTTATCACGCATGATGTT |
| | TCATCTGCAATTAACAGGCCACAAATAGGCGTGGTAAGAGAATTCCTTACACGTAACCCTGCTTGGA |
| | GAAAAGCTGTCTTTATTTCACCTTATAATTCACAGAATGCTGTAGCCTCAAAGATTTTGGGACTACC |
| | AACTCAAACTGTTGATTCATCACAGGGCTCAGAATATGACTATGTCATATTCACTCAAACCACTGAA |
| | ACAGCTCACTCTTGTAATGTAAACAGATTTAATGTTGCTATTACCAGAGCAAAAGTAGGCATACTTT |
| | GCATAATGTCTGATAGAGACCTTTATGACAAGTTGCAATTTACAAGTCTTGAAATTCCACGTAGGAA |
| | TGTGGCAACTTTACAAGCTGAAAATGTAACAGGACTTTTTAAAGATTGTAGTAAGGTAATCACTGGG |
| | TTACATCCTACACAGGCACCTACACACCTCAGTGTTGACACTAAATTCAAAACTGAAGGTTTATGTG |
| | TTGACATACCTGGCATACCTAAGGACATGACCTATAGAAGACTCATCTCTATGATGGGTTTTAAAAT |
| | GAATTATCAAGTTAATGGTTACCCTAACATGTTTATCACCCGCGAAGAAGCTATAAGACATGTACGT |
| | GCATGGATTGGCTTCGATGTCGAGGGGTGTCATGCTACTAGAGAAGCTGTTGGTACCAATTTACCTT |
| | TACAGCTAGGTTTTTCTACAGGTGTTAACCTAGTTGCTGTACCTACAGGTTATGTTGATACACCTAA |
| | TAATACAGATTTTTCCAGAGTTAGTGCTAAACCACCGCCTGGAGATCAATTTAAACACCTCATACCA |
| | CTTATGTACAAAGGACTTCCTTGGAATGTAGTGCGTATAAAGATTGTACAAATGTTAAGTGACACAC |
| | TTAAAAATCTCTCTGACAGAGTCGTATTTGTCTTATGGGCACATGGCTTTGAGTTGACATCTATGAA |
| | GTATTTTGTGAAAATAGGACCTGAGCGCACCTGTTGTCTATGTGATAGACGTGCCACATGCTTTTCC |
| | ACTGCTTCAGACACTTATGCCTGTTGGCATCATTCTATTGGATTTGATTACGTCTATAATCCGTTTA |
| | TGATTGATGTTCAACAATGGGGTTTTACAGGTAACCTACAAAGCAACCATGATCTGTATTGTCAAGT |
| | CCATGGTAATGCACATGTAGCTAGTTGTGATGCAATCATGACTAGGTGTCTAGCTGTCCACGAGTGC |
| | TTTGTTAAGCGTGTTGACTGGACTATTGAATATCCTATAATTGGTGATGAACTGAAGATTAATGCGG |
| | CTTGTAGAAAGGTTCAACACATGGTTGTTAAAGCTGCATTATTAGCAGACAAATTCCCAGTTCTTCA |
| | CGACATTGGTAACCCTAAAGCTATTAAGTGTGTACCTCAAGCTGATGTAGAATGGAAGTTCTATGAT |
| | GCACAGCCTTGTAGTGACAAAGCTTATAAAATAGAAGAATTATTCTATTCTTATGCCACACATTCTG |
| | ACAAATTCACAGATGGTGTATGCCTATTTTGGAATTGCAATGTCGATAGATATCCTGCTAATTCCAT |
| | TGTTTGTAGATTTGACACTAGAGTGCTATCTAACCTTAACTTGCCTGGTTGTGATGGTGGCAGTTTG |
| | TATGTAAATAAACATGCATTCCACACACCAGCTTTTGATAAAAGTGCTTTTGTTAATTTAAAACAAT |
| | TACCATTTTTCTATTACTCTGACAGTCCATGTGAGTCTCATGGAAAACAAGTAGTGTCAGATATAGA |
| | TTATGTACCACTAAAGTCTGCTACGTGTATAACACGTTGCAATTTAGGTGGTGCTGTCTGTAGACAT |
| | CATGCTAATGAGTACAGATTGTATCTCGATGCTTATAACATGATGATCTCAGCTGGCTTTAGCTTGT |
| | GGGTTTACAAACAATTTGATACTTATAACCTCTGGAACACTTTTACAAGACTTCAGAGTTTAGAAAA |
| | TGTGGCTTTTAATGTTGTAAATAAGGGACACTTTGATGGACAACAGGGTGAAGTACCAGTTTCTATC |
| | ATTAATAACACTGTTTACACAAAAGTTGATGGTGTTGATGTAGAATTGTTTGAAAATAAAACAACAT |
| | TACCTGTTAATGTAGCATTTGAGCTTTGGGCTAAGCGCAACATTAAACCAGTACCAGAGGTGAAAAT |
| | ACTCAATAATTTGGGTGTGGACATTGOTGCTAATACTGTGATCTGGGACTACAAAAGAGATGOTCCA |
| | GCACATATATCTACTATTGGTGTTTGTTCTATGACTGACATAGCCAAGAAACCAACTGAAACGATTT |
| | GTGCACCACTCACTGTCTTTTTTGATGGTAGAGTTGATGGTCAAGTAGACTTATTTAGAAATGCCCG |
| | TAATGGTGTTCTTATTACAGAAGGTAGTGTTAAAGGTTTACAACCATCTGTAGGTCCCAAACAAGCT |
| | AGTCTTAATGGAGTCACATTAATTGGAGAAGCCGTAAAAACACAGTTCAATTATTATAAGAAAGTTG |
| | ATGGTGTTGTCCAACAATTACCTGAAACTTACTTTACTCAGAGTAGAAATTTACAAGAATTTAAACC |
| | CAGGAGTCAAATGGAAATTGATTTCTTAGAATTAGCTATGGATGAATTCATTGAACGGTATAAATTA |
| | GAAGGCTATGCCTTCGAACATATCGTTTATGGAGATTTTAGTCATAGTCAGTTAGGTGGTTTACATC |
| | TACTGATTGGACTAGCTAAACGTTTTAAGGAATCACCTTTTGAATTAGAAGATTTTATTCCTATGGA |
| | CAGTACAGTTAAAAACTATTTCATAACAGATGCGCAAACAGGTTCATCTAAGTGTGTGTGTTCTGTT |
| | ATTGATTTATTACTTGATGATTTTGTTGAAATAATAAAATCCCAAGATTTATCTGTAGTTTCTAAGG |
| | TTGTCAAAGTGACTATTGACTATACAGAAATTTCATTTATGCTTTGGTGTAAAGATGGCCATGTAGA |
| | AACATTTTACCCAAAATTACAATCTAGTCAAGCGTGGCAACCGGGTGTTGCTATGCCTAATCTTTAC |
| | AAAATGCAAAGAATGCTATTAGAAAAGTGTGACCTTCAAAATTATGGTGATAGTGCAACATTACCTA |
| | AAGGCATAATGATGAATGTCGCAAAATATACTCAACTGTGTCAATATTTAAACACATTAACATTAGC |
| | TGTACCCTATAATATGAGAGTTATACATTTTGGTGCTGGTTCTGATAAAGGAGTTGCACCAGGTACA |
| | GCTGTTTTAAGACAGTGGTTGCCTACGGGTACGCTGCTTGTCGATTCAGATCTTAATGACTTTGTCT |
| | CTGATGCAGATTCAACTTTGATTGGTGATTGTGCAACTGTACATACAGCTAATAAATGGGATCTCAT |
| | TATTAGTGATATGTACGACCCTAAGACTAAAAATGTTACAAAAGAAAATGACTCTAAAGAGGGTTTT |
| | TTCACTTACATTTGTGGGTTTATACAACAAAAGCTAGCTCTTGGAGGTTCCGTGGCTATAAAGATAA |
| | CAGAACATTCTTGGAATGCTGATCTTTATAAGCTCATGGGACACTTCGCATGGTGGACAGCCTTTGT |
| | TACTAATGTGAATGCGTCATCATCTGAAGCATTTTTAATTGGATGTAATTATCTTGGCAAACCACGC |
| | GAACAAATAGATGGTTATGTCATGCATGCAAATTACATATTTTGGAGGAATACAAATCCAATTCAGT |
| | TGTCTTCCTATTCTTTATTTGACATGAGTAAATTTCCCCTTAAATTAAGGGGTACTGCTGTTATGTC |
| | TTTAAAAGAAGGTCAAATCAATGATATGATTTTATCTCTTCTTAGTAAAGGTAGACTTATAATTAGA |
| | GAAAACAACAGAGTTGTTATTTCTAGTGATGTTCTTGTTAACAACTAAACGAACAATGTTTGTTTTT |
| | CTTGTTTTATTGCCACTAGTCTCTAGTCAGTGTGTTAATCTTACAACCAGAACTCAATTACCCCCTG |
| | CATACACTAATTCTTTCACACGTGGTGTTTATTACCCTGACAAAGTTTTCAGATCCTCAGTTTTACA |
| | TTCAACTCAGGACTTGTTCTTACCTTTCTTTTCCAATGTTACTTGGTTCCATGCTATACATGTCTCT |
| | GGGACCAATGGTACTAAGAGGTTTGATAACCCTGTCCTACCATTTAATGATGGTGTTTATTTTGCTT |
| | CCACTGAGAAGTCTAACATAATAAGAGGCTGGATTTTTGGTACTACTTTAGATTCGAAGACCCAGTC |
| | CCTACTTATTGTTAATAACGCTACTAATGTTGTTATTAAAGTCTGTGAATTTCAATTTTGTAATGAT |
| | CCATTTTTGGGTGTTTATTACCACAAAAACAACAAAAGTTGGATGGAAAGTGAGTTCAGAGTTTATT |
| | CTAGTGCGAATAATTGCACTTTTGAATATGTCTCTCAGCCTTTTCTTATGGACCTTGAAGGAAAACA |
| | GGGTAATTTCAAAAATCTTAGGGAATTTGTGTTTAAGAATATTGATGGTTATTTTAAAATATATTCT |
| | AAGCACACGCCTATTAATTTAGTGCGTGATCTCCCTCAGGGTTTTTCGGCTTTAGAACCATTGGTAG |
| | ATTTGCCAATAGGTATTAACATCACTAGGTTTCAAACTTTACTTGCTTTACATAGAAGTTATTTGAC |
| | TCCTGGTGATTCTTCTTCAGGTTGGACAGCTGGTGCTGCAGCTTATTATGTGGGTTATCTTCAACCT |
| | AGGACTTTTCTATTAAAATATAATGAAAATGGAACCATTACAGATGCTGTAGACTGTGCACTTGACC |
| | CTCTCTCAGAAACAAAGTGTACGTTGAAATCCTTCACTGTAGAAAAAGGAATCTATCAAACTTCTAA |
| | CTTTAGAGTCCAACCAACAGAATCTATTGTTAGATTTCCTAATATTACAAACTTGTGCCCTTTTGGT |
| | GAAGTTTTTAACGCCACCAGATTTGCATCTGTTTATGCTTGGAACAGGAAGAGAATCAGCAACTGTG |
| | TTGCTGATTATTCTGTCCTATATAATTCCGCATCATTTTCCACTTTTAAGTGTTATGGAGTGTCTCC |
| | TACTAAATTAAATGATCTCTGCTTTACTAATGTCTATGCAGATTCATTTGTAATTAGAGGTGATGAA |
| | GTCAGACAAATCGCTCCAGGGCAAACTGGAAAGATTGCTGATTATAATTATAAATTACCAGATGATT |
| | TTACAGGCTGCGTTATAGCTTGGAATTCTAACAATCTTGATTCTAAGGTTGGTGGTAATTATAATTA |
| | CCTGTATAGATTGTTTAGGAAGTCTAATCTCAAACCTTTTGAGAGAGATATTTCAACTGAAATCTAT |
| | CAGGCCGGTAGCACACCTTGTAATGGTGTTGAAGGTTTTAATTGTTACTTTCCTTTACAATCATATG |
| | GTTTCCAACCCACTAATGGTGTTGGTTACCAACCATACAGAGTAGTAGTACTTTCTTTTGAACTTCT |
| | ACATGCACCAGCAACTGTTTGTGGACCTAAAAAGTCTACTAATTTGGTTAAAAACAAATGTGTCAAT |
| | TTCAACTTCAATGGTTTAACAGGCACAGGTGTTCTTACTGAGTCTAACAAAAAGTTTCTGCCTTTCC |
| | AACAATTTGGCAGAGACATTGCTGACACTACTGATGCTGTCCGTGATCCACAGACACTTGAGATTCT |
| | TGACATTACACCATGTTCTTTTGGTGGTGTCAGTGTTATAACACCAGGAACAAATACTTCTAACCAG |
| | GTTGCTGTTCTTTATCAGGATGTTAACTGCACAGAAGTCCCTGTTGCTATTCATGCAGATCAACTTA |
| | CTCCTACTTGGCGTGTTTATTCTACAGGTTCTAATGTTTTTCAAACACGTGCAGGCTGTTTAATAGG |
| | GGCTGAACATGTCAACAACTCATATGAGTGTGACATACCCATTGGTGCAGGTATATGCGCTAGTTAT |
| | CAGACTCAGACTAATTCTCCTCGGCGGGCACGTAGTGTAGCTAGTCAATCCATCATTGCCTACACTA |
| | TGTCACTTGGTGCAGAAAATTCAGTTGCTTACTCTAATAACTCTATTGCCATACCCACAAATTTTAC |
| | TATTAGTGTTACCACAGAAATTCTACCAGTGTCTATGACCAAGACATCAGTAGATTGTACAATGTAC |
| | ATTTGTGGTGATTCAACTGAATGCAGCAATCTTTTGTTGCAATATGGCAGTTTTTGTACACAATTAA |
| | ACCGTGCTTTAACTGGAATAGCTGTTGAACAAGACAAAAACACCCAAGAAGTTTTTGCACAAGTCAA |
| | ACAAATTTACAAAACACCACCAATTAAAGATTTTGGTGGTTTTAATTTTTCACAAATATTACCAGAT |
| | CCATCAAAACCAAGCAAGAGGTCATTTATTGAAGATCTACTTTTCAACAAAGTGACACTTGCAGATG |
| | CTGGCTTCATCAAACAATATGGTGATTGCCTTGGTGATATTGCTGCTAGAGACCTCATTTGTGCACA |
| | AAAGTTTAACGGCCTTACTGTTTTGCCACCTTTGCTCACAGATGAAATGATTGCTCAATACACTTCT |
| | GCACTGTTAGCGGGTACAATCACTTCTGGTTGGACCTTTGGTGCAGGTGCTGCATTACAAATACCAT |
| | TTGCTATGCAAATGGCTTATAGGTTTAATGGTATTGGAGTTACACAGAATGTTCTCTATGAGAACCA |
| | AAAATTGATTGCCAACCAATTTAATAGTGCTATTGGCAAAATTCAAGACTCACTTTCTTCCACAGCA |
| | AGTGCACTTGGAAAACTTCAAGATGTGGTCAACCAAAATGCACAAGCTTTAAACACGCTTGTTAAAC |
| | AACTTAGCTCCAATTTTGGTGCAATTTCAAGTGTTTTAAATGATATCCTTTCACGTCTTGACAAAGT |
| | TGAGGCTGAAGTGCAAATTGATAGGTTGATCACAGGCAGACTTCAAAGTTTGCAGACATATGTGACT |
| | CAACAATTAATTAGAGCTGCAGAAATCAGAGCTTCTGCTAATCTTGCTGCTACTAAAATGTCAGAGT |
| | GTGTACTTGGACAATCAAAAAGAGTTGATTTTTGTGGAAAGGGCTATCATCTTATGTCCTTCCCTCA |
| | GTCAGCACCTCATGGTGTAGTCTTCTTGCATGTGACTTATGTCCCTGCACAAGAAAAGAACTTCACA |
| | ACTGCTCCTGCCATTTGTCATGATGGAAAAGCACACTTTCCTCGTGAAGGTGTCTTTGTTTCAAATG |
| | GCACACACTGGTTTGTAACACAAAGGAATTTTTATGAACCACAAATCATTACTACAGACAACACATT |
| | TGTGTCTGGTAACTGTGATGTTGTAATAGGAATTGTCAACAACACAGTTTATGATCCTTTGCAACCT |
| | GAATTAGACTCATTCAAGGAGGAGTTAGATAAATATTTTAAGAATCATACATCACCAGATGTTGATT |
| | TAGGTGACATCTCTGGCATTAATGCTTCAGTTGTAAACATTCAAAAAGAAATTGACCGCCTCAATGA |
| | GGTTGCCAAGAATTTAAATGAATCTCTCATCGATCTCCAAGAACTTGGAAAGTATGAGCAGTATATA |
| | AAATGGCCATGGTACATTTGGCTAGGTTTTATAGCTGGCTTGATTGCCATAGTAATGGTGACAATTA |
| | TGCTTTGCTGTATGACCAGTTGCTGTAGTTGTCTCAAGGGCTGTTGTTCTTGTGGATCCTGCTGCAA |
| | ATTTGATGAAGACGACTCTGAGCCAGTGCTCAAAGGAGTCAAATTACATTACACATAAACGAACTTA |
| | TGGATTTGTTTATGAGAATCTTCACAATTGGAACTGTAACTTTGAAGCAAGGTGAAATCAAGGATGC |
| | TACTCCTTCAGATTTTGTTCGCGCTACTGCAACGATACCGATACAAGCCTCACTCCCTTTCGGATGG |
| | CTTATTGTTGGCGTTGCACTTCTTGCTGTTTTTCAGAGCGCTTCCAAAATCATAACCCTCAAAAAGA |
| | GATGGCAACTAGCACTCTCCAAGGGTGTTCACTTTGTTTGCAACTTGCTGTTGTTGTTTGTAACAGT |
| | TTACTCACACCTTTTGCTCGTTGCTGCTGGCCTTGAAGCCCCTTTTCTCTATCTTTATGCTTTAGTC |
| | TACTTCTTGCAGAGTATAAACTTTGTAAGAATAATAATGAGGCTTTGGCTTTGCTGGAAATGCCGTT |
| | CCAAAAACCCATTACTTTATGATGCCAACTATTTTCTTTGCTGGCATACTAATTGTTACGACTATTG |
| | TATACCTTACAATAGTGTAACTTCTTCAATTGTCATTACTTCAGGTGATGGCACAACAAGTCCTATT |
| | TCTGAACATGACTACCAGATTGGTGGTTATACTGAAAAATGGGAATCTGGAGTAAAAGACTGTGTTG |
| | TATTACACAGTTACTTCACTTCAGACTATTACCAGCTGTACTCAACTCAATTGAGTACAGACACTGG |
| | TGTTGAACATGTTACCTTCTTCATCTACAATAAAATTGTTGATGAGCCTGAAGAACATGTCCAAATT |
| | CACACAATCGACGGTTCATCCGGAGTTGTTAATCCAGTAATGGAACCAATTTATGATGAACCGACGA |
| | CGACTACTAGCGTGCCTTTGTAAGCACAAGCTGATGAGTACGAACTTATGTACTCATTCGTTTCGGA |
| | AGAGACAGGTACGTTAATAGTTAATAGCGTACTTCTTTTTCTTGCTTTCGTGGTATTCTTGCTAGTT |
| | ACACTAGCCATCCTTACTGCGCTTCGATTGTGTGCGTACTGCTGCAATATTGTTAACGTGAGTCTTG |
| | TAAAACCTTCTTTTTACGTTTACTCTCGTGTTAAAAATCTGAATTCTTCTAGAGTTCCTGATCTTCT |
| | GGTCTAAACGAACTAAATATTATATTAGTTTTTCTGTTTGGAACTTTAATTTTAGCCATGGCAGATT |
| | CCAACGGTACTATTACCGTTGAAGAGCTTAAAAAGCTCCTTGAACAATGGAACCTAGTAATAGGTTT |
| | CCTATTCCTTACATGGATTTGTCTTCTACAATTTGCCTATGCCAACAGGAATAGGTTTTTGTATATA |
| | ATTAAGTTAATTTTCCTCTGGCTGTTATGGCCAGTAACTTTAGCTTGTTTTGTGCTTGCTGCTGTTT |
| | ACAGAATAAATTGGATCACCGGTGGAATTGCTATCGCAATGGCTTGTCTTGTAGGCTTGATGTGGCT |
| | CAGCTACTTCATTGCTTCTTTCAGACTGTTTGCGCGTACGCGTTCCATGTGGTCATTCAATCCAGAA |
| | ACTAACATTCTTCTCAACGTGCCACTCCATGGCACTATTCTGACCAGACCGCTTCTAGAAAGTGAAC |
| | TCGTAATCGGAGCTGTGATCCTTCGTGGACATCTTCGTATTGCTGGACACCATCTAGGACGCTGTGA |
| | CATCAAGGACCTGCCTAAAGAAATCACTGTTGCTACATCACGAACGCTTTCTTATTACAAATTGGGA |
| | GCTTCGCAGCGTGTAGCAGGTGACTCAGGTTTTGCTGCATACAGTCGCTACAGGATTGGCAACTATA |
| | AATTAAACACAGACCATTCCAGTAGCAGTGACAATATTGCTTTGCTTGTACAGTAAGTGACAACAGA |
| | TGTTTCATCTCGTTGACTTTCAGGTTACTATAGCAGAGATATTACTAATTATTATGAGGACTTTTAA |
| | AGTTTCCATTTGGAATCTTGATTACATCATAAACCTCATAATTAAAAATTTATCTAAGTCACTAACT |
| | GAGAATAAATATTCTCAATTAGATGAAGAGCAACCAATGGAGATTGATTAAACGAACATGAAAATTA |
| | TTCTTTTCTTGGCACTGATAACACTCGCTACTTGTGAGCTTTATCACTACCAAGAGTGTGTTAGAGG |
| | TACAACAGTACTTTTAAAAGAACCTTGCTCTTCTGGAACATACGAGGGCAATTCACCATTTCATCCT |
| | CTAGCTGATAACAAATTTGCACTGACTTGCTTTAGCACTCAATTTGCTTTTGCTTGTCCTGACGGCG |
| | TAAAACACGTCTATCAGTTACGTGCCAGATCAGTTTCACCTAAACTGTTCATCAGACAAGAGGAAGT |
| | TCAAGAACTTTACTCTCCAATTTTTCTTATTGTTGCGGCAATAGTGTTTATAACACTTTGCTTCACA |
| | CTCAAAAGAAAGACAGAATGATTGAACTTTCATTAATTGACTTCTATTTGTGCTTTTTAGCCTTTCT |
| | GCTATTCCTTGTTTTAATTATGCTTATTATCTTTTGGTTCTCACTTGAACTGCAAGATCATAATGAA |
| | ACTTGTCACGCCTAAACGAACATGAAATTTCTTGTTTTCTTAGGAATCATCACAACTGTAGCTGCAT |
| | TTCACCAAGAATGTAGTTTACAGTCATGTACTCAACATCAACCATATGTAGTTGATGACCCGTGTCC |
| | TATTCACTTCTATTCTAAATGGTATATTAGAGTAGGAGCTAGAAAATCAGCACCTTTAATTGAATTG |
| | TGCGTGGATGAGGCTGGTTCTAAATCACCCATTCAGTACATCGATATCGGTAATTATACAGTTTCCT |
| | GTTCACCTTTTACAATTAATTGCCAGGAACCTAAATTGGGTAGTCTTGTAGTGCGTTGTTCGTTCTA |
| | TGAAGACTTTTTAGAGTATCATGACGTTCGTGTTGTTTTAGATTTCATCTAAACGAACAAACTAAAA |
| | TGTCTGATAATGGACCCCAAAATCAGCGAAATGCACCCCGCATTACGTTTGGTGGACCCTCAGATTC |
| | AACTGGCAGTAACCAGAATGGAGAACGCAGTGGGGCGCGATCAAAACAACGTCGGCCCCAAGGTTTA |
| | CCCAATAATACTGCGTCTTGGTTCACCGCTCTCACTCAACATGGCAAGGAAGACCTTAAATTCCCTC |
| | GAGGACAAGGCGTTCCAATTAACACCAATAGCAGTCCAGATGACCAAATTGGCTACTACCGAAGAGC |
| | TACCAGACGAATTCGTGGTGGTGACGGTAAAATGAAAGATCTCAGTCCAAGATGGTATTTCTACTAC |
| | CTAGGAACTGGGCCAGAAGCTGGACTTCCCTATGGTGCTAACAAAGACGGCATCATATGGGTTGCAA |
| | CTGAGGGAGCCTTGAATACACCAAAAGATCACATTGGCACCCGCAATCCTGCTAACAATGCTGCAAT |
| | CGTGCTACAACTTCCTCAAGGAACAACATTGCCAAAAGGCTTCTACGCAGAAGGGAGCAGAGGCGGC |
| | AGTCAAGCCTCTTCTCGTTCCTCATCACGTAGTCGCAACAGTTCAAGAAATTCAACTCCAGGCAGCA |
| | GTAGGGGAACTTCTCCTGCTAGAATGGCTGGCAATGGCGGTGATGCTGCTCTTGCTTTGCTGCTGCT |
| | TGACAGATTGAACCAGCTTGAGAGCAAAATGTCTGGTAAAGGCCAACAACAACAAGGCCAAACTGTC |
| | ACTAAGAAATCTGCTGCTGAGGCTTCTAAGAAGCCTCGGCAAAAACGTACTGCCACTAAAGCATACA |
| | ATGTAACACAAGCTTTCGGCAGACGTGGTCCAGAACAAACCCAAGGAAATTTTGGGGACCAGGAACT |
| | AATCAGACAAGGAACTGATTACAAACATTGGCCGCAAATTGCACAATTTGCCCCCAGCGCTTCAGCG |
| | TTCTTCGGAATGTCGCGCATTGGCATGGAAGTCACACCTTCGGGAACGTGGTTGACCTACACAGGTG |
| | CCATCAAATTGGATGACAAAGATCCAAATTTCAAAGATCAAGTCATTTTGCTGAATAAGCATATTGA |
| | CGCATACAAAACATTCCCACCAACAGAGCCTAAAAAGGACAAAAAGAAGAAGGCTGATGAAACTCAA |
| | GCCTTACCGCAGAGACAGAAGAAACAGCAAACTGTGACTCTTCTTCCTGCTGCAGATTTGGATGATT |
| | TCTCCAAACAATTGCAACAATCCATGAGCAGTGCTGACTCAACTCAGGCCTAAACTCATGCAGACCA |
| | CACAAGGCAGATGGGCTATATAAACGTTTTCGCTTTTCCGTTTACGATATATAGTCTACTCTTGTGC |
| | AGAATGAATTCTCGTAACTACATAGCACAAGTAGATGTAGTTAACTTTAATCTCACATAGCAATCTT |
| | TAATCAGTGTGTAACATTAGGGAGGACTTGAAAGAGCCACCACATTTTCACCGAGGCCACGCGGAGT |
| | ACGATCGAGTGTACAGTGAACAATGCTAGGGAGAGCTGCCTATATGGAAGAGCCCTAATGTGTAAAA |
| | TTAATTTTAGTAGTGCTATCCCCATGTGATTTTAATAGCTTCTTAGGAGAATGACAAAAAAAAAAAA |

This application hereby incorporates by reference PCT/US2020/019924 (which was published as WO 2020/176638) in its entirety, particularly with respect to luciferase enzymes and reactions, polymerase extension reactions and techniques, the coupled enzymatic reaction involving glyceraldehyde 3-phosphate dehydrogenase and phosphoglycerate kinase to produce ATP, the enzymatic reaction involving adenosine 5′-phosphosulfate and free phosphates in the presence of adenosine triphosphate sulfurylase to produce ATP, quantification and quantitation of bioluminescent readout signal, multiplexing, solid supports, enzyme immobilization techniques, control reactions, and kits.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

### EXAMPLES

The examples below are intended to exemplify the practice of embodiments of the disclosure but are by no means intended to limit the scope thereof.

### Example 1—Design of ID-Oligo Molecules that Target Strain/Virus Specific Domains

The ID-Oligo design process begins with the identification of genomic regions that are free of uracil or thymine (RNA or DNA respectively). Uracil/thymine-free regions were selected with their length in mind (e.g., sequence greater than 15, 20, 22, 25, or 30 nucleotides in length) (Table 10), because longer stretches theoretically produce a higher signal (FIG. 1B). This guiding process simplifies/streamlines/accelerates the selection of regions to serve as the complement for the ID-Oligos.

TABLE 10
Selected Uracil/Thymine-Free Region Positions with
SARS-CoV-Urbani Genome

| Region Length | Uracil/Thymine-Free Region Position within the | | | | | | | |
|---|---|---|---|---|---|---|---|---|
| (Base Pairs) | SARS-CoV-Urbani Viral Genome | | | | | | | |
| 15 | 29,604 | 29,582 | 29,414 | 29,282 | 29,241 | 29,225 | 28,971 | 28,845 |
| | 28,651 | 28,557 | 27,638 | 26,744 | 24,676 | 24,607 | 24,203 | 22,696 |
| | 22,524 | 21,449 | 21,239 | 20,025 | 19,827 | 19,613 | 18,205 | 17,563 |
| | 17,395 | 12,858 | 12,264 | 10,129 | 9,958 | 8,406 | 6,299 | 6,201 |
| | 5,159 | 4,353 | 3,816 | 3,437 | 3,252 | 3,198 | 3,066 | 2,658 |
| | 2,478 | 1,812 | 1,024 | 893 | 302 | 32 | | |
| 20 | 29,287 | 29,246 | 28,976 | 28,850 | 28,656 | 28,241 | 19,832 | 19,618 |
| | 12,863 | 3,821 | 3,203 | 37 | | | | |
| 22 | 29,289 | 29,248 | 28,852 | 28,658 | 28,243 | 19,834 | 3,823 | 39 |
| 25 | 29,292 | 28,855 | 28,661 | 28,246 | 3,826 | | | |
| 30 | 28,251 | | | | | | | |

Next, the ID-Oligo molecules were designed to bind adjacent to a uracil/thymine-free region in the target SARS-CoV-2 genome. Table 11 lists regions 3′ to uracil/thymine-free sequences (organized by position on the genome) (SEQ ID NOs:10-89), which are potential targets for ID-Oligos for detection of the SARS-CoV-2 virus. Also listed in Table 11 are the sequences for the ID-Oligos (SEQ ID NOs:90-169), as well as the uracil/thymine free template sequences (SEQ ID NOs:170-249).

TABLE 11
SARS-CoV-2 Genomic Regions 3′ to Uracil/Thymine-Free Sequences

| | | | SEQ ID | | SEQ ID | ID-Oligo | | SEQ ID | T-Free |
|---|---|---|---|---|---|---|---|---|---|
| position | ID-Oligo NAME | Region for ID-Oligo | NO: | ID-Oligo (rev + comp) | NO: | (Length) | T-Free seq | NO: | (Length) |
| 27096 | SARS-CoV-2A#80 | TTCCAGTAGCAGTGACAA | 10 | TTGTCACTGCTACTGGAA | 90 | 18 | AAACACAGACCA | 170 | 12 |
| 26217 | SARS-CoV-2A#79 | TACGTTAATAGTTAATAGCGT | 11 | CGCTATTAACTATTAACGTA | 91 | 20 | CGGAAGAGACAGG | 171 | 13 |
| 26144 | SARS-CoV-2A#78 | TACTAGCGTGCCTTTGTAA | 12 | TTACAAAGGCACGCTAGTA | 92 | 19 | GAACCGACGACGAC | 172 | 14 |
| 24867 | SARS-CoV-2A#77 | TTTGTGTCTGGTAACTGTGA | 13 | TCACAGTTACCAGACACAAA | 93 | 20 | ACAGACAACACA | 173 | 12 |
| 24830 | SARS-CoV-2A#76 | TTTTTATGAACCACAAATC | 14 | GATTTGTGGTTCATAAAAA | 94 | 19 | AACACAAAGGAA | 174 | 12 |
| 24771 | SARS-CoV-2A#75 | TTTCCTCGTGAAGGTGTC | 15 | GACACCTTCACGAGGAAA | 95 | 18 | GGAAAAGCACAC | 175 | 12 |
| 24726 | SARS-CoV-2A#74 | TTCACAACTGCTCCTGCCAT | 16 | ATGGCAGGAGCAGTTGTGAA | 96 | 20 | GCACAAGAAAAGAAC | 176 | 15 |
| 24270 | SARS-CoV-2A#73 | TTGATTGCCAACCAATTTA | 17 | TAAATTGGTTGGCAATCAA | 97 | 19 | GAGAACCAAAAA | 177 | 12 |
| 23947 | SARS-CoV-2A#72 | TCATTTATTGAAGATCTA | 18 | TAGATCTTCAATAAATGA | 98 | 18 | CAAAACCAAGCAAGAGG | 178 | 17 |
| 23884 | SARS-CoV-2A#71 | TTAAAGATTTTGGTGGTTTTAA | 19 | AAAACCACCAAAATCTTTAA | 99 | 20 | ACAAAACACCACCAA | 179 | 15 |
| 23835 | SARS-CoV-2A#70 | TTTTTGCACAAGTCAAACAAA | 20 | TTGTTTGACTTGTGCAAAAA | 100 | 20 | GAACAAGACAAAAACAC | 180 | 25 |
| | | | | | | | CCAAGAAG | | |
| 23312 | SARS-CoV-2A#69 | TACTTCTAACCAGGTTGCT | 21 | AGCAACCTGGTTAGAAGTA | 101 | 19 | AACACCAGGAACAAA | 181 | 15 |
| 23156 | SARS-CoV-2A#68 | TGTTCTTACTGAGTCTAACA | 22 | TGTTAGACTCAGTAAGAACA | 102 | 20 | AACAGGCACAGG | 182 | 12 |
| 22576 | SARS-CoV-2A#67 | TCAGCAACTG TGTTGCTGAT | 23 | TCAGCAACACAGTTGCTGA | 103 | 20 | AACAGGAAGAGAA | 183 | 14 |
| 22545 | SARS-CoV-2A#66 | TTTGCATCTGTTTATGC | 24 | GCATAAACAGATGCAAA | 104 | 17 | AACGCCACCAGA | 184 | 12 |
| 22478 | SARS-CoV-2A#65 | TCTATTGTTAGATTTCCTAA | 25 | TTAGGAAATCTAACAATAGA | 105 | 20 | CCAACCAACAGAA | 185 | 14 |
| 22056 | SARS-CoV-2A#64 | TAATTTCAAAAATCTTAGGG | 26 | CCCTAAGATTTTTGAAATTA | 106 | 20 | GAAGGAAAACAGGG | 186 | 14 |
| 21952 | SARS-CoV-2A#63 | TTGGATGGAAAGTGAGTTCA | 27 | TGAACTCACTTTCCATCCAA | 107 | 20 | ACCACAAAAACAACAAA | 187 | 19 |
| | | | | | | | AG | | |
| 21461 | SARS-CoV-2A#62 | TTGTTATTTCTAGTGATG | 28 | CATCACTAGAAATAACAA | 108 | 18 | AGAGAAAACAACAGAG | 188 | 16 |
| 21251 | SARS-CoV-2A#61 | TAGATGGTTATGTCATGCAT | 29 | ATGCATGACATAACCATCTA | 109 | 20 | GGCAAACCACGCGAAC | 189 | 19 |
| | | | | | | | AAA | | |
| 19897 | SARS-CoV-2A#60 | TGAAACGATTTGTGCACCA | 30 | TGGTGCACAAATCGTTTCA | 110 | 19 | AGCCAAGAAACCAAC | 190 | 15 |
| 19273 | SARS-CoV-2A#59 | TTTTGATAAAAGTGCTTT | 31 | AAAGCACTTTTATCAAAA | 111 | 18 | CCACACACCAGC | 191 | 12 |
| 18754 | SARS-CoV-2A#58 | TGATCTGTATTGTCAAGTC | 32 | GACTTGACAATACAGATCA | 112 | 19 | ACAAAGC AACCA | 192 | 13 |
| 18217 | SARS-CoV-2A#57 | TATAAGACATGTACGTGCA | 33 | TGCACGTACATGTCTTATA | 113 | 19 | CACCCGCGAAGAAGC | 193 | 15 |
| 17888 | SARS-CoV-2A#56 | TAGGCATACTTTGCATAATG | 34 | CATTATGCAAAGTATGCCTA | 114 | 20 | ACCAGAGCAAAAG | 194 | 13 |
| 17657 | SARS-CoV-2A#55 | TAGGCGTGGTAAGAGAATTC | 35 | GAATTCTCTTACCACGCCTA | 115 | 20 | AACAGGCCACAAA | 195 | 13 |
| 17411 | SARS-CoV-2A#54 | TTGCTAACTAAGGGCACA | 36 | TGTGCCCTTAGTTAGCAA | 116 | 18 | GCACCACGCACA | 196 | 12 |
| 17285 | SARS-CoV-2A#53 | TATAGTTGTCTTTGATGAA | 37 | TTCATCAAAGACAACTATA | 117 | 19 | GAGACGACAGCAGA | 197 | 14 |
| 17032 | SARS-CoV-2A#52 | TGGTACTGGTAAGAGTCA | 38 | TGACTCTTACCAGTACCA | 118 | 18 | CCAGGGACCACC | 198 | 12 |
| 16915 | SARS-CoV-2A#51 | TATGTTAGAATTACTGGC | 39 | GCCAGTAATTCTAACATA | 119 | 18 | GCCACAAGAGCAC | 199 | 13 |
| 16592 | SARS-CoV-2A#50 | TCAAAGCTACTGAGGAGACA | 40 | TGTCTCCTCAGTAGCTTTGA | 120 | 20 | GCAGCAGAAACGC | 200 | 13 |
| 15164 | SARS-CoV-2A#49 | TTCTATGGTGGTTGGCAC | 41 | GTGCCAACCACCATAGAA | 121 | 18 | GGAACAAGCAAA | 201 | 12 |
| 14221 | SARS-CoV-2A#48 | TCTTTGACCGTTATTTTAAA | 42 | TTTAAAATAACGGTCAAAGA | 122 | 20 | CACGGAAGAGAGGTTAA | 202 | 20 |
| | | | | | | | AAC | | |
| 14065 | SARS-CoV-2A#47 | TAGTGGAGTTCCTGTTGTAG | 43 | CTACAACAGGAACTCCACTA | 123 | 20 | ACAAACCACGCCAGG | 203 | 15 |
| 13894 | SARS-CoV-2A#46 | TATATTACGCGTATACGCC | 44 | GGCGTATACGCGTAATATA | 124 | 19 | AGAAAACCCAGA | 204 | 12 |
| 13461 | SARS-CoV-2A#45 | TGACAATTTAATTGATTCT | 45 | AGAATCAATTAAATTGTCA | 125 | 19 | CCAAGAAAAGGACGAA | 205 | 18 |
| | | | | | | | GA | | |
| 13152 | SARS-CoV-2A#44 | TAGTACTGATGTCGTATACA | 46 | TGTATACGACATCAGTACTA | 126 | 20 | GCGGCACAGGCAC | 206 | 13 |
| 13080 | SARS-CoV-2A#43 | TATGGATCAAGAATCCTTT | 47 | AAAGGATTCTTGATCCATA | 127 | 19 | ACACCGGAAGCCAA | 207 | 14 |
| 12736 | SARS-CoV-2A#42 | TCACTAATTGTGTTAAGA | 48 | TCTTAACACAATTAGTGA | 128 | 18 | GGGGGACAACCAA | 208 | 13 |
| 12414 | SARS-CoV-2A#41 | TAGGTTTGTACTTGCACT | 49 | AGTGCAAGTACAAACCTA | 129 | 18 | ACAACACAACAAAGGGA | 209 | 19 |
| | | | | | | | GG | | |
| 12276 | SARS-CoV-2A#40 | TAATGGTTGTCATACCAGAC | 50 | GTCTGGTATGACAACCATTA | 130 | 20 | ACAACAGCAGCCAAAC | 210 | 16 |
| 12026 | SARS-CoV-2A#39 | TTACTAGTGCTATGCAGAC | 51 | GTCTGCATAGCACTAGTAA | 131 | 19 | GAGGACAAGAGGGCA | 211 | 20 |
| | | | | | | | AAAG | | |
| 11708 | SARS-CoV-2A#38 | TTACAAGCTATAGCCTCAG | 52 | CTGAGGCTATAGCTTGTAA | 132 | 19 | GGACAACAGGGCAACC | 212 | 16 |
| 11306 | SARS-CoV-2A#37 | TAGCATAGATGCCTTCAAACT | 53 | GTTTGAAGGCATCTATGCTA | 133 | 20 | GACTACTCCCACCCAAG | 213 | 19 |
| | | | | | | | AA | | |
| 10736 | SARS-CoV-2A#36 | TGTGTATGATGATGGTGCTA | 54 | TAGCACCATCATCATACACA | 134 | 20 | GACAGCAAGAAC | 214 | 12 |
| 10569 | SARS-CoV-2A#35 | TGTTGACATACTAGGACCTC | 55 | GAGGTCCTAGTATGTCAACA | 135 | 20 | AACACAAGACCA | 215 | 12 |
| 10329 | SARS-CoV-2A#34 | TGGTACGGACACAACTATT | 56 | AATAGTTGTGTCCGTACCA | 136 | 19 | GACAGGCAAACAGCAC | 216 | 23 |
| | | | | | | | AAGCAGC | | |
| 9970 | SARS-CoV-2A#33 | TTTTTCAGTGTTAGCTTGT | 57 | ACAAGCTAACACTGAAAAA | 137 | 19 | TTCGCATTCAACCAGGA | 217 | 22 |
| | | | | | | | CAGAC | | |
| 7470 | SARS-CoV-2A#32 | TCTATCACCTCAGCTGTTTT | 58 | AAAACAGCTGAGGTGATAGA | 138 | 20 | ACCAACCACCACAAACC | 218 | 17 |
| 6310 | SARS-CoV-2A#31 | TCGAATGTACAACTATTG | 59 | CAATAGTTGTACATTCGA | 139 | 18 | AGAGCAACAAGAG | 219 | 13 |
| 6262 | SARS-CoV-2A#30 | TGGATAATCTTGCCTGCGA | 60 | TCGCAGGCAAGATTATCCA | 140 | 19 | CAGAGGACGCGCAGGG | 220 | 18 |
| | | | | | | | AA | | |
| 5982 | SARS-CoV-2A#29 | TTGAAACATCAAATTCGTT | 61 | AACGAATTTGATGTTTCAA | 141 | 19 | GGAGCACAAAACCAG | 221 | 15 |
| 5966 | SARS-CoV-2A#28 | TATCCAAACGCAAGCTTCGA | 62 | TCGAAGCTTGCGTTTGGATA | 142 | 20 | ACCAAACCAACCA | 222 | 13 |
| 5942 | SARS-CoV-2A#27 | TAAAACCAGTTACTTATAAA | 63 | TTTATAAGTAACTGGTTTTA | 143 | 20 | ACACAACAACCA | 223 | 12 |
| 5839 | SARS-CoV-2A#26 | TTACACAACAACCATAAAAC | 64 | GTTTTATGGTTGTTGTGTAA | 144 | 20 | ACAAAGAAAACAG | 224 | 13 |
| 5824 | SARS-CoV-2A#25 | TTAAGGGTGTAGAAGCTGTTA | 65 | AACAGCTTCTACACCCTTAA | 145 | 20 | GGACAACAGCAGACAA | 225 | 19 |
| | | | | | | | CCC | | |
| 5487 | SARS-CoV-2A#24 | TGGTGAAGCTGCTAACTT | 66 | AAGTTAGCAGCTTCACCA | 146 | 18 | ACAGAGCAAGGGC | 226 | 13 |
| 5320 | SARS-CoV-2A#23 | TTAATGCCTGTCTGTGTGG | 67 | CCACACAGACAGGCATTAA | 147 | 19 | GCAGAAGAAACACGCA | 227 | 19 |
| | | | | | | | AA | | |
| 4365 | SARS-CoV-2A#22 | TTCTTGGAACTGTTTCTTGG | 68 | CCAAGAAACAGTTCCAAGAA | 148 | 20 | GAGAAGCAAGAAA | 228 | 13 |
| 4308 | SARS-CoV-2A#21 | TGCTTAAAAAGTGTAAAAG | 69 | CTTTTACACTTTTTAAGCA | 149 | 19 | AGAGGAGGCAAAGACA | 229 | 17 |
| | | | | | | | G | | |
| 4238 | SARS-CoV-2A#20 | TGATAAGAAAATCAAAGCTTGT | 70 | AAGCTTTGATTTTCTTATCA | 150 | 20 | GAACAGAGAAAACAAGA | 230 | 17 |
| 4181 | SARS-CoV-2A#19 | TCTTGCTAAACACTGTCTTCA | 71 | GAAGACAGTGTTTAGCAAGA | 151 | 20 | AAGCGGACACAA | 231 | 12 |
| 3888 | SARS-CoV-2A#18 | TGAGGACAATCAGACAAC | 72 | GTTGTCTGATTGTCCTCA | 152 | 18 | CAACAAGACGGCAG | 232 | 14 |
| 3536 | SARS-CoV-2A#17 | TTGGTTAGATGATGATAGTC | 73 | GACTATCATCATCTAACCAA | 153 | 20 | GAAGAAGAGCAAGAAG | 233 | 20 |
| | | | | | | | AAGA | | |
| 3189 | SARS-CoV-2A#16 | TTGAGCCATCAACTCAATA | 74 | TATTGAGTTGATGGCTCAA | 154 | 19 | GAAGAAGAAGAGT | 234 | 13 |
| 3135 | SARS-CoV-2A#15 | TTACTTTTGGTGATGACA | 75 | TGTCATCACCAAAAGTAA | 155 | 18 | GCACCAACAAAGG | 235 | 13 |
| 3030 | SARS-CoV-2A#14 | TACTGTGCCCTTGCACCTAA | 76 | TTAGGTGCAAGGGCACAGTA | 156 | 20 | CAAAGACACAGAAAAG | 236 | 16 |
| 2676 | SARS-CoV-2A#13 | TTCCCACAGAAGTGTTAACA | 77 | TGTTAACACTTCTGTGGGAA | 157 | 20 | AGAGGGAGAAACAC | 237 | 14 |
| 2600 | SARS-CoV-2A#12 | TACTGTGCCCTTGCACCTAATA | 78 | TATTAGGTGCAAGGGCACAG | 158 | 22 | CAAAAGCCCCAAAAGAA | 238 | 18 |
| | | | | TA | | | A | | |
| 2447 | SARS-CoV-2A#11 | TTCCCACAGAAGTGTTAACA | 79 | TGTTAACACTTCTGTGGGAA | 159 | 20 | AGAGGGAGAAACAC | 239 | 14 |
| 2420 | SARS-CoV-2A#10 | TTATCTTCTTAGAGGGAGAA | 80 | TTCTCCCTCTAAGAAGATAA | 160 | 20 | AAAAGCCCCAAAAGAAA | 240 | 17 |
| 2389 | SARS-CoV-2A#9 | TGGCCTACTCATGCCTCTA | 81 | TAGAGGCATGAGTAGGCCA | 161 | 19 | CCAGAGAAGAAAC | 241 | 13 |
| 1907 | SARS-CoV-2A#8 | TATAACAATACTAGATGGAATT | 82 | TTCCATCTAGTATTGTTATA | 162 | 20 | ACAGAAGGCCGC | 242 | 12 |
| 1755 | SARS-CoV-2A#7 | TAAAAAAGGTGCCTGGAA | 83 | TTCCAGGCACCTTTTTTA | 163 | 18 | ACAAAAGGAAAAGC | 243 | 14 |
| 1583 | SARS-CoV-2A#6 | TCAACATCAATATTGTTGG | 84 | CCAACAATATTGATGTTGA | 164 | 19 | CCAAAAAGAGAAAG | 244 | 14 |
| 1213 | SARS-CoV-2A#5 | TTTTGTTAAAGCCACTTGCGA | 85 | CGCAAGTGGCTTTAACAAAA | 165 | 20 | GGCAGACGGGCGA | 245 | 13 |
| 1086 | SARS-CoV-2A#4 | TTGATGGCTTTATGGGTA | 86 | TACCCATAAAGCCATCAA | 166 | 18 | GAAAAGAAAAAGC | 246 | 13 |
| 753 | SARS-CoV-2A#3 | TACACTCGCTATGTCGATAA | 87 | TTATCGACATAGCGAGTGTA | 167 | 20 | TTAACGGAGG GGCA | 247 | 15 |
| 245 | SARS-CoV-2A#2 | TCCAACTCAGTTTGCCTGT | 88 | ACAGGCAAACTGAGTTGGA | 168 | 19 | CAACGAGAAAACACAC | 248 | 18 |
| | | | | | | | G | | |
| 111 | SARS-CoV-2A#1 | TAACTCGTCTATCTTCTGCA | 89 | TGCAGAAGATAGACGAGTTA | 169 | 20 | GACAGGACAC GAG | 249 | 14 |

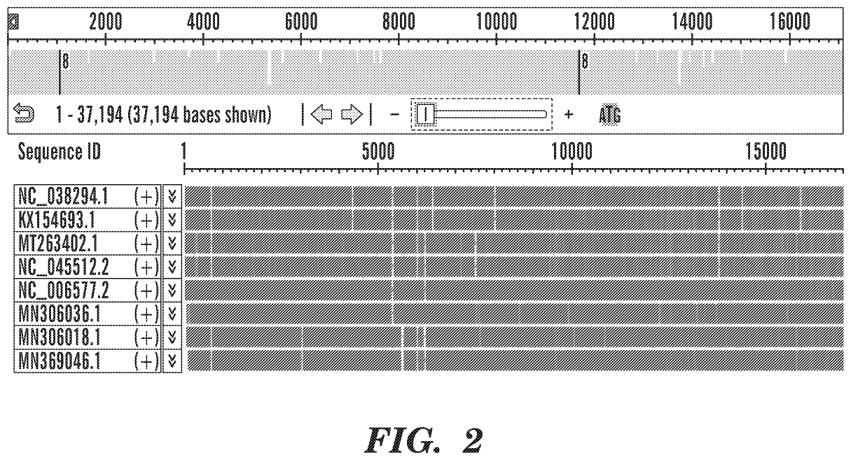

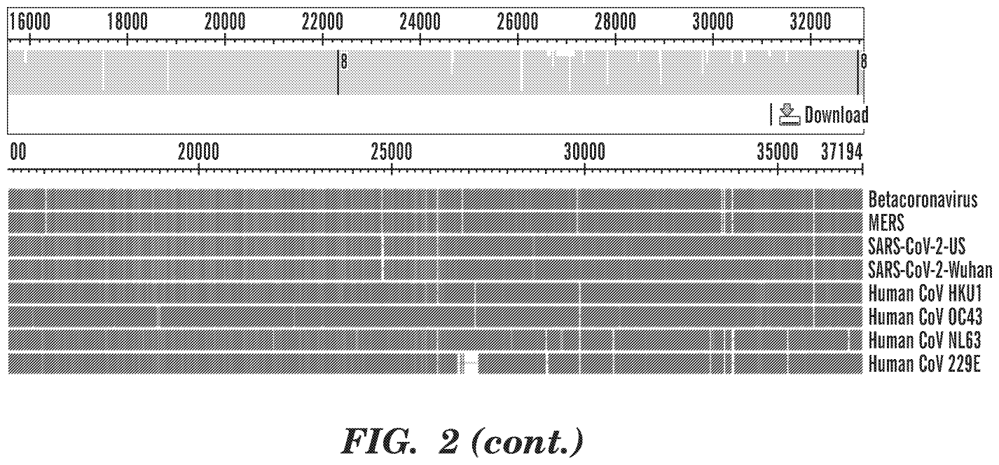

Alignment analysis of sequences from Table 11 was used to further evaluate the selected ID-Oligo molecules (SEQ ID NOs: 90-169), based on their location in regions that are unique and/or conserved with the target viral genome. FIG. 2 shows an alignment of the target SARS-CoV-2 genome against genomes from 7 other corona viruses (e.g., MERS, SARS-CoV-2, CoV-HKU1, CoV-OC43, CoV-NL63, and CoV-229E).

### Example 2—Human Coronavirus 229E ID-Oligo Optimization

Various Oligo-IDs designed for the detection of Human Coronavirus 229E (Human CoV-229E) are identified in Table 12 below.

TABLE 12
Human CoV-229E ID-Oligos

| Name | Sequence | SEQ ID NO: |
|---|---|---|
| HCV229E1 | GTGGAAAAAGTTCAATCCA | 251 |
| HCV229E2 | CAAAAGAAACAGGGTTAACA | 252 |
| HCV229E3 | AACTCAAAATTGGTGTTAAA | 253 |
| HCV229E4 | GTTGAACATTATTATCTAAA | 254 |
| HCV229E5 | AAGACTACGTTTTAAAACAACA | 255 |
| HCV229E6 | GCATGCCAAACAATAGGA | 256 |
| HCV229E7 | GCAACAGCATTTTCATA | 257 |
| HCV229E8 | CAAAATCAGAGGCCAAACAAGTA | 258 |
| HCV229E9 | GCCTCCATCACCTTCA | 259 |
| HCV229E10 | CTTTACACAATTGCCAACA | 260 |
| HCV229E11 | CTTTACACAATTGCCAACA | 261 |
| HCV229E12 | AAACAGAATTACGGTTTAA | 262 |
| HCV229E13 | CTTGTAAATGGTAGAATA | 263 |
| HCV229E14 | GTTTTCTCAACACACTCCAA | 264 |
| HCV229E15 | GTTTAGAAAAAATGAAGCAA | 265 |
| HCV229E16 | CCAATAGCCTATAAGCTTA | 266 |
| HCV229E17 | GTAACTTGGGTGACAAATCCA | 267 |
| HCV229E18 | GTACCCGTTTTCGCTGA | 268 |
| HCV229E19 | ACCGTGGCTTTTGCATTTCA | 269 |

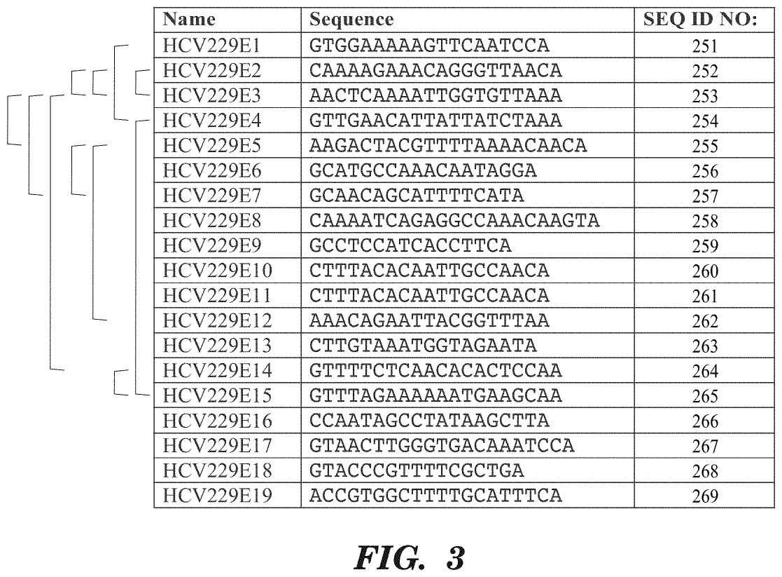

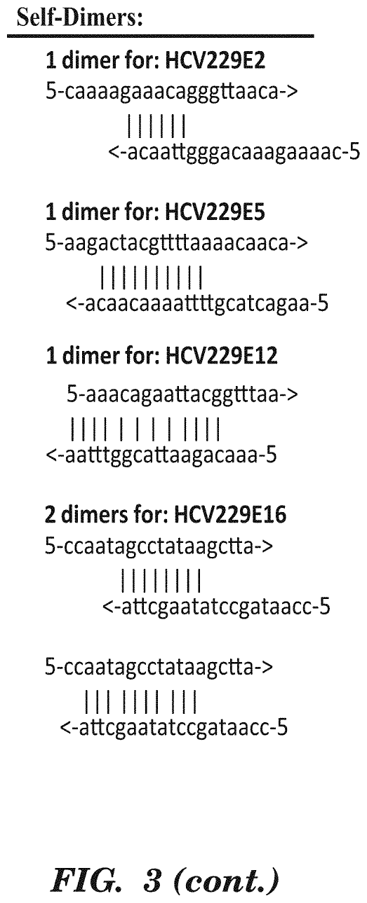

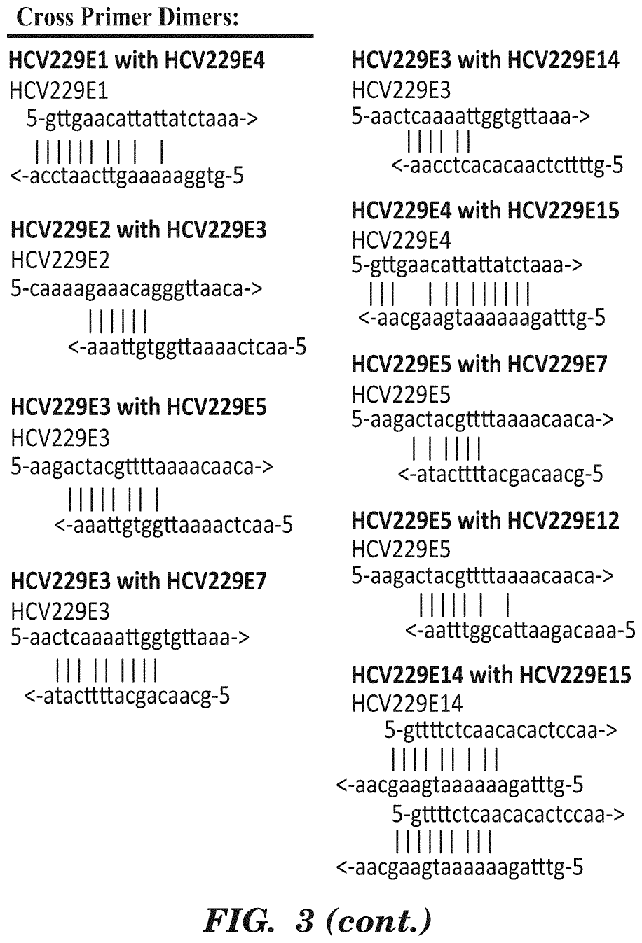

The optimal mixture of ID-Oligos for use in a single reaction was determined by using analytical tools that provide information regarding self-dimerization and cross-dimerization. FIG. 3 identifies (i) HCV229E2 (SEQ ID NO:252), HCV229E5 (SEQ ID NO:255), HCV229E12 (SEQ ID NO:262), and HCV229E16 (SEQ ID NO:266) as forming self-dimers and (ii) HCV229E1 (SEQ ID NO:251) as forming a dimer with HCV229E4 (SEQ ID NO:254); HCV229E2 (SEQ ID NO:252) as forming a dimer with HCV229E3 (SEQ ID NO:253); HCV229E3 (SEQ ID NO:253) as forming dimers with HCV229E5 (SEQ ID NO:255), HCV229E7 (SEQ ID NO:257), and HCV229E14 (SEQ ID NO:264); HCV229E4 (SEQ ID NO:254) as forming a dimer with HCV229E15 (SEQ ID NO:265); HCV229E5 (SEQ ID NO:255) as forming dimers with HCV229E7 (SEQ ID NO:257) and HCV229E12 (SEQ ID NO:262); and HCV229E14 (SEQ ID NO:264) as forming a dimer with HCV229E15 (SEQ ID NO:265).

### Example 3—Detection of Synthetic Fragments of Human SARS-CoV-Urbani

To demonstrate the use of ID-Oligos (i.e., primers or oligonucleotide molecules complementary to a selected portion of a target at least partially single-stranded viral genome) to detect a target at least partially single-stranded viral genome, three synthetic fragments of the SARS-CoV-Urbani were generated (FIG. 6A). The synthetic genomes were used as the target at least partially single-stranded viral genome identified in FIG. 4. Each synthetic sequence included a uracil/thymine-free region and a target sequence for the ID-Oligo to bind. Target sequences are shown in Table 13 below.

TABLE 13
Target Sequence for ID-Oligo Binding

| Binding | | |
|---|---|---|
| Sequence | | Reverse Complement |
| Name | Sequence | of Sequence |
| SARS-C- | TTCCAAAACTGAGGAGAA | CAGATTTCTCCTCAGTTT |
| U#1 | ATCTG | TGGAA |
| | (SEQ ID NO: 270) | (SEQ ID NO: 273) |
| SARS-C- | TGTCACTAAGAAATCTGC | GCAGCAGATTTCTTAGTG |
| U#2 | TGC | AGA |
| | (SEQ ID NO: 271) | (SEQ ID NO: 274) |
| SARS-C- | TGATGAAGCTCAGCCTTT | GCAAAGGCTGAGCTTCAT |
| U#3 | GC | CA |
| | (SEQ ID NO: 272) | (SEQ ID NO: 275) |

The synthetic SARS-CoV-Urbani genome fragments were characterized, as shown in FIG. 6B. A master mix comprising His-Si-Luc (0.8 μl), ATPS (NEB, 0.8 μl), BST (DNA Polymerase, 0.8 μl, NEB), buffer (HEPES, MgCl₂, NaCl, pH7.4), luciferin (1 mM), APS (0.06 mM), and dNTP mix (0.05 mM of each dTTP, dCTP, dGTP) was combined with the synthetic genome fragments. FIG. 6C shows the luminescent signal generated by the reaction according to the methods described herein in the presence of each of the 3 synthetic genome fragments, and negative controls (reactions lacking the synthetic genome fragments). These results demonstrate the ability of ID-Oligos to detect a target at least partially single-stranded viral genome.

### Example 4—Comparison of Three Different Reverse Transcriptase Enzymes to Detect Zika Virus Genome

To evaluate the ability of different reverse transcriptase (RT) enzymes to detect a target at least partially single-stranded viral genome, 3 different RT enzymes were evaluated against RNA isolated from Zika virus. The 3 RT enzymes used were ProtoScript® II Reverse Transcriptase, M-MuLV Reverse Transcriptase, and Avian Myeloblastosis Virus (AMV) Reverse Transcriptase. ProtoScript® II Reverse Transcriptase is a recombinant M-MuLV reverse transcriptase with reduced RNase H activity and increased thermostability. It can be used to synthesize first strand cDNA at higher temperatures than the wild type M-MuLV. The enzyme is active up to 48° C., providing higher specificity, higher yield of cDNA and more full-length cDNA product up to 12 kb. The gene encoding M-MuLV Reverse Transcriptase is expressed in E. coli in a vector that results in 16 additional amino acids at the N-terminus and 13 amino acids at the C-terminus. This construct results in a fully functional Reverse Transcriptase protein with a functional RNase H domain. AMV RT is an RNA-directed DNA polymerase. This enzyme can synthesize a complementary DNA strand initiating from a primer using RNA (cDNA synthesis) or single-stranded DNA as a template.

FIG. 7A shows that the AMV RT with active RNAse-H activity demonstrated a delayed onset of the luminescence signals compared to both the ProtoScript® II and M-MuLV RT, which lack RNAse-H activity. Analysis of assay kinetics for each RT enzyme reaction revealed slightly faster rates for the ProtoScript® II RT (mutated form of the AMV RT enzyme) than the AMV RT or M-MuLV RT (FIG. 7B).

### Example 5—ID-Oligos can Distinguish Between Zika Virus and HCV Genomes

Next, whether ID-Oligos designed to complement a specific target single-stranded viral genome were selective for the target single-stranded genome was evaluated. A set of ID-Oligos specific for regions within the Zika virus genome were mixed with the assay reagents, which included His-Si-Luc (20 μl), ATPS (NEB, 1 μl), buffer (HEPES, MgCl₂, NaCl, pH7.4), luciferin (10 mM), APS (0.06 mM), dNTP mix (0.05 mM of each dTTP, dCTP, dGTP). The master mix was then added to individual wells in a 96-well microplate containing RNA from Zika virus (strain KX26887.1), Hepatitis C virus (strain JFH-1), or water, and primers designed to identify the Zika RNA. Luminescent signal was integrated every 0.4 seconds, and measured for up to 50 minutes at room temperature. The data in FIG. 8 shows that using Zika-specific ID-Oligos did not result in cross-reactivity with RNA from Hepatitis C virus. Both Zika and Hepatitis C are RNA viruses in the same viral family (Flaviviridae).

### Example 6—ID-Oligos Detect Target Single-Stranded Viral Genomes in Serum and Saliva

To determine whether ID-Oligos can be used to detect a target single-stranded viral genome in serum and saliva samples, assay reagent mixtures comprising His-Si-Luc (20 μl), ATPS (NEB, 1 μl), Klenow (1 μl, NEB), buffer (HEPES, MgCl₂, NaCl, pH7.4), luciferin (10 mM), APS (0.06 mM), dNTP mix (0.05 mM of each dTTP, dCTP, dGTP), and oligos (1 micromolar of each of 3 oligos) were prepared as a Master mix. The master mix was added to individual wells in a 96-well microplate containing ssDNA diluted in saliva or serum, and luminescence signal was measured for up to 50 minutes (at room temperature). FIG. 9 shows that the signal detected in the saliva sample was greater than that detected in the serum sample.

### Example 7—Reaction Kinetics for Detection of RNA Isolated from the Common Human Coronavirus 229E in the Presence of Various ID-Oligos

19 different ID-Oligos were added to the reaction mixture in the presence of 0.1 pg RNA isolated from human coronavirus 229E (ATCC). The reaction mixture included His-Si-Luc (20 μl), ATPS (NEB, 1 μl), ProtoScript® II (1 NEB), buffer (HEPES, MgCl₂, NaCl, pH7.4), luciferin (10 mM), APS (0.06 mM), dNTP mix (0.05 mM of each dTTP, dCTP, dGTP), and oligos (as indicated, 1 micromolar of each). Master mix was added to individual wells in a 96-well microplate containing RNA or water (as indicated) and luminescence signal was measured for up to 50 minutes (at room temperature).

The reaction kinetics in the presence of individual ID-Oligos 1-19 (corresponding to SEQ ID NOs:251-269) are shown in FIGS. 10A-10D. Reaction kinetics in the presence of mixtures of ID-Oligos, or in the presence of a set of ID-Oligos designed to detect the Zika virus are shown in FIG. 10E. A summary of the reaction kinetics (slope) in the presence of each ID-Oligo and combinations of ID-Oligos is shown in FIG. 10F. The results in FIG. 10E illustrate the specificity of the ID-Oligos designed to detect coronavirus 229E.

### Example 8—Use of dATPαS Enables Inclusion of Oligos Targeting Non-Uracil/Thymine Free Regions in a Target Viral Genome

Whether using dATPαS in the reaction mixture would enable the inclusion of ID-Oligos designed to target non-uracil/thymine-free regions in a target viral genome was next evaluated. Structural differences between dATP and dATPαS is shown in FIG. 11A.

A master mix for 5 reactions was prepared by combining His-Si-Luc (5 μl), buffer (HEPES, MgCl₂, NaCl, pH7.4), luciferin (10 mM), APS, dNTP mix (0.06 mM of each dTTP, dCTP, dGTP), and either 0.1 mM/1 mM of dATP, 0.1 mM/1 mM of dATPαS or water. FIG. 11B shows the reduction in the LU background when using dATPαS versus dATP, which provides a significantly improved signal-to-noise ratio (˜2orders of magnitude) and thus higher overall sensitivity of the assay.

Next, a master mix for reactions was prepared by combining His-Si-Luc (5 μl), ATPS (NEB, 0.2 μl), ProtoScript® II RT (NEB, 0.2 μl), buffer (HEPES, MgCl₂, NaCl, pH7.4), luciferin (10 mM), APS (0.06 mM), dNTP mix (0.05 mM of each dTTP, dCTP, dGTP), +/−0.1 mM dATPαS, and ID-Oligos mix (ID-Oligos corresponding to SEQ ID NOs:30-48 of Table 11 (i.e., ID-Oligos having the sequence of SEQ ID NOs:110-128 of Table 11)+ID-Oligos corresponding to SEQ ID NOs:69-89 of Table 11 (i.e., ID-Oligos having the sequence of SEQ ID NOs:149-169 of Table 11). FIG. 11C shows a comparison of the reaction signal output with only dC/T/G or with dC/T/C+dATPαS. Blue bars—reaction slopes when only dT/G/C's are included. Red bars—reaction slopes when supplemented with dATPαS. Higher slope values for the reaction carried out using dATPαS indicates increased dynamic assay kinetics due to the longer polymerization reaction step by the RT.

### Example 9—Inhibitory Compounds

Compounds and reagents that inhibit or interfere with the methods described herein include Na-azide, Na-citrate, heparin, EDTA, EGTA, proteases, phosphates, 2,2′-azino-di-(3-ethylbenzothiazoline)-6-sulfonic acid (ABTS), and butylated hydroxytoluene (BHT). FIG. 12 demonstrate the inhibitory effect of Na-azide on the luminescence output of the coupled ATPS-luciferase reaction.

### Example 10—Oligo-ID Preparation/Purification

To evaluate whether ID-Oligos add any background LU due to carry over of PPi produced during their synthesis process, ID-Oligo purification via HPLC and standard desalting (salt exchange) were evaluated in comparison to non-purified ID-Oligos (control). Master mix was prepared comprising His-Si-Luc (10 μl), buffer (HEPES, MgCl₂, NaCl, pH7.4), luciferin (10 mM), ATPS (1 NEB), and APS (0.33 mM). 97 μl of master mix was added to wells (96-well microplate) containing 3 μl of 100 μM of ID-Oligos, or water (no oligo). LU was measured for up to 50 minutes. FIG. 13 demonstrates that different methods of ID-Oligo purification do not add to background luminescence.

### Example 11—Detecting Synthetic SARS-CoV-2 Using a 2-Step Oligo-ID Assay

Next, a two-step Oligo-ID assay was carried out using free-enzymes or nanoparticle-immobilized enzymes. In brief, the RT (Mu-MLV) was mixed with ID-Oligos, reagents, and RNA for an initial hybridization/polymerization step. Next, the luciferase (Luc) and ATPS were added to transduce PPi formation (by the RT) into ATP and luminescence. Master mix for 2 reactions included: His-Si-MuMLV (7 μl), buffer (HEPES, MgCl₂, NaCl, pH7.4), luciferin (10 mM), oligo (CV3-oligo, 1 μM), APS (0.33 mM), and RNA (synthetic SARS-CoV-2 (CV3, 0.3 μM) or water (“-RNA” control reaction). 88 μl of master mix was added to wells (96-well microplate), and LU was measured for 250-300 seconds (Step I). At 250/300 second, a mix of His-Si-Luc (5 μl) and His-Si-ATPS (6 μl) was added to both wells to initiate step II of the reaction. FIG. 14A shows the results of the Oligo-ID assays using free enzymes in solution. FIG. 14B shows the results of the Oligo-ID assay using enzymes attached to nanoparticles (NPs) (500 nm, SiO₂).

### Example 12—Different ATPS/RT Ratios Affect Reaction Kinetics

To determine whether different ATPS/RT rations affect reaction kinetics, a master mix for comprising increasing volumes of ATPS was prepared. The master mix comprised: His-Si-Luc (10 μl), buffer (HEPES, MgCl₂, NaCl, pH7.4), luciferin (10 mM), APS (0.33 mM), dNTP mix (0.05 mM of each dTTP, dCTP, dGTP, and dATPαS), 40× oligo mix, SARS-CoV-2 RNA (#N-52285, 1:500 dilution), Mu-MLV RT (0.2 μl NEB[200 U/μl]) and increasing volumes of ATPS (NEB [3 U/μl]). The master mix was then added to wells (96-well microplate) containing ATPS or water (no oligo) to result in the following unit ratios of ATPS/RT: 0, 0.0015, 0.003, 0.0075, 0.015, 0.03, 0.075. LU was measured for up to 50 minutes. FIG. 15 shows that optimal LU was detected at an ATPS/RT ratio of 0.03.

### Example 13—Detection of SARS-CoV-2 RNA in Saliva and Nasopharyngeal Samples

To examine whether the ID-Oligos assay could be used to detect SARS-CoV-2 RNA in saliva or nasopharyngeal samples from patients diagnosed with COVID-19, paired saliva (S) and nasopharyngeal (NP) samples were collected from patients known to have confirmed COVID-19 infection (Guthrie Medical Center, Sayre, Pa.). Viral RNA was extracted from individual samples using the MagMAX Viral/Pathogen II Nucleic Acid Isolation Kit (Applied Biosystems, CA), and eluted in water. Because the extracted RNA samples had magnetic beads carried over, a second round of cleaning was performed using the RNA Clean&Concentrate kit (Zymo Research, CA) and eluted in water. A master mix for 9 reactions was prepared, which included His-Si-Luc (5 μl), His-Si-ATPS (6 μl), His-Si-MuMLV (7 μl), buffer (HEPES, MgCl₂, NaCl, pH7.4), luciferin (10 mM), APS (0.06 mM), dNTP mix (0.05 mM of each dTTP, dCTP, dGTP, and dATPαS), and 40× oligo mix (ID-Oligos corresponding to SEQ ID NOs:30-48 of Table 11 (i.e., ID-Oligos having the sequence of SEQ ID NOs:110-128 of Table 11)+ID-Oligos corresponding to SEQ ID NOs:69-89 of Table 11 (i.e., ID-Oligos having the sequence of SEQ ID NOs:149-169 of Table 11). The master mix was added to individual 96-well microplate wells containing 0, 1, or 4 μl of saliva RNA extract, or 0, 1, 5, 10, 30 μl of nasopharyngeal (NP) RNA extract. The plate was inserted into a TECAN Infinite Pro plate reader (at 35° C.) and luminescence was integrated at intervals of 400 ms from each well continually (up to 50 minutes). FIGS. 16A-16C demonstrate that SARS-CoV-2 RNA can be detected in saliva and nasopharyngeal samples from COVID-19 patients.

### Example 14—Detection of SARS-CoV-2 RNA in Pooled Saliva

To examine whether the assay according to methods described herein could be used to detect SARS-CoV-2 RNA in a pooled saliva sample, saliva samples from patients diagnosed with COVID-19 were pooled. In brief, RNA extracted from saliva (S) of 4 donors were pooled into a single sample (collected from patients known to have confirmed COVID-19 infection (Guthrie Medical Center, Sayer Pa.). Viral RNA was extracted from individual samples using the MagMAX Viral/Pathogen II Nucleic Acid Isolation Kit (Applied Biosystems, CA), and eluted in water. Because the extracted RNA samples had magnetic beads carried over, a second round of cleaning was performed using the RNA Clean&Concentrate kit (Zymo Research, CA) and eluted in water. A master was prepared for 2 reactions. The master mix included His-Si-Luc (5 μl), His-Si-ATPS (6 μl), His-Si-MuMLV (7 μl), buffer (HEPES, MgCl₂, NaCl, pH7.4), luciferin (10 mM), APS (0.06 mM), dNTP mix (0.05 mM of each dTTP, dCTP, dGTP, and dATPαS), and 40× oligo mix (ID-Oligos corresponding to SEQ ID NOs:30-48 of Table 11 (i.e., ID-Oligos having the sequence of SEQ ID NOs:110-128 of Table 11)+ID-Oligos corresponding to SEQ ID NOs:69-89 of Table 11 (i.e., ID-Oligos having the sequence of SEQ ID NOs:149-169 of Table 11). The master mix was then added to individual 96-well microplate wells containing the pooled saliva sample, or negative control (water). The plate was inserted into a TECAN Infinite Pro plate reader (at 35° C.) and luminescence was integrated at intervals of 400 ms from each well continually (up to 50 minutes). FIG. 17 shows the change in luminescence units (LU, normalized to time point 0) over time for the pooled saliva sample as compared to a negative control.

### Example 15—Detection of SARS-CoV-2 RNA in a Nasopharyngeal (NP) Sample

To examine whether the assay according to methods described herein could detect SARS-CoV-2 RNA in nasopharyngeal (NP) samples, RNA extracted from nasopharyngeal (NP) samples collected from 1 confirmed Covid-19 positive donor (#17) and 1 confirmed negative donor (#14) Guthrie Medical Center, Sayre, Pa.) were evaluated. Viral RNA was extracted from individual samples using the MagMAX Viral/Pathogen II Nucleic Acid Isolation Kit (Applied Biosystems, CA), and eluted in water. A master mix for 2 reactions included: His-Si-Luc (5 μl), His-Si-ATPS (6 μl), His-Si-MuMLV (7 μl), buffer (HEPES, MgCl₂, NaCl, pH7.4), luciferin (10 mM), APS (0.06 mM), dNTP mix (0.05 mM of each dTTP, dCTP, dGTP, and dATPαS), and 40× oligo mix (ID-Oligos corresponding to SEQ ID NOs:30-48 of Table 11 (i.e., ID-Oligos having the sequence of SEQ ID NOs:110-128 of Table 11)+ID-Oligos corresponding to SEQ ID NOs:69-89 of Table 11 (i.e., ID-Oligos having the sequence of SEQ ID NOs:149-169 of Table 11). The master mix was then added to individual 96-well microplate wells containing the pooled saliva sample, or negative control (water). The plate was inserted into a TECAN Infinite Pro plate reader (at 35° C.) and luminescence was integrated at intervals of 400 ms from each well continually (up to 50 minutes). FIG. 18 shows the change in luminescence units (LU, normalized to time point 0) over time.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of detecting a target at least partially single-stranded viral genome in a sample, said method comprising:
providing a sample containing a target at least partially single-stranded viral genome;
providing an oligonucleotide molecule complementary to a selected portion of the target at least partially single-stranded viral genome adjacent to a single-stranded template region of the target at least partially single-stranded viral genome;
contacting the sample with the oligonucleotide molecule so that the oligonucleotide molecule hybridizes to the selected portion of the target at least partially single-stranded viral genome and forms a hybridization product comprising a double-stranded nucleic acid start portion in the viral genome at a location corresponding to the selected portion and the single-stranded template region is adjacent to the double-stranded nucleic acid start portion;
contacting the hybridization product with a polymerase and a dNTP mixture to form a polymerase extension mixture;
subjecting the polymerase extension mixture to conditions under which the hybridization product from the double-stranded nucleic acid start portion is extended by addition of nucleotides complementary to the single-stranded template region, thereby releasing free phosphates;
producing adenosine triphosphates from the released free phosphates; and
metabolizing the adenosine triphosphates produced from the free phosphates to produce a readout signal, indicating the presence of the target at least partially single-stranded viral genome in the sample.

2. The method of claim 1, wherein the single-stranded template region is a uracil/thymine-free region.

3. The method of claim 1, wherein the target at least partially single-stranded viral genome comprises RNA.

4. The method of claim 1, wherein the polymerase is a reverse transcriptase.

5. The method of claim 4, wherein the reverse transcriptase is selected from the group consisting of Protoscript® II Reverse Transcriptase (Protoscript® II RT), Maloney Murine Leukemia Virus Reverse Transcriptase (M-MuLV RT), and Avian Myeloblastosis Virus Reverse Transcriptase (AMV RT).

6. The method of claim 1, wherein the target at least partially single-stranded viral genome comprises DNA.

7. The method of claim 1, wherein the polymerase is a DNA polymerase.

8. The method of claim 1, wherein the polymerase is coupled to a solid support.

9. The method of claim 8, wherein the polymerase is coupled to the solid support with a linker selected from the group consisting of His-Si, His, Si, biotin, streptavidin, Pt, Au, Ag, His-Pt, His-Au, His-Ag, GST, an antibody, and an epitope tag.

10. The method of claim 1, wherein luciferase metabolizes the adenosine triphosphates to produce a readout signal that is a bioluminescent readout signal.

11. The method of claim 10, wherein the luciferase is coupled to a solid support.

12. The method of claim 11, wherein the luciferase is coupled to the solid support with a linker selected from the group consisting of His-Si, His, Si, biotin, streptavidin, Pt, Au, Ag, His-Pt, His-Au, His-Ag, GST, an antibody, and an epitope tag.

13. The method of claim 1, wherein said producing adenosine triphosphates comprises:
subjecting the released free phosphates to a coupled glyceraldehyde 3-phosphate dehydrogenase-phosphoglycerate kinase enzymatic reaction to produce adenosine triphosphate.

14. The method of claim 13, wherein said subjecting the released free phosphates to a coupled glyceraldehyde 3-phosphate dehydrogenase-phosphoglycerate kinase enzymatic reaction comprises:
contacting adenosine diphosphate, nicotinamide adenine dinucleotide and glyceraldehyde 3-phosphate to achieve the coupled glyceraldehyde 3-phosphate dehydrogenase-phosphoglycerate kinase enzymatic reaction.

15. The method of claim 14, wherein the glyceraldehyde 3-phosphate dehydrogenase and the phosphoglycerate kinase are coupled to a solid support.

16. The method of claim 1, wherein said producing adenosine triphosphates comprises:
contacting the released free phosphates with adenosine 5′-phosphosulfate in the presence of adenosine triphosphate sulfurylase to produce adenosine triphosphate.

17. The method of claim 16, wherein the adenosine triphosphate sulfurylase is coupled to a solid support.

18. The method of claim 1, wherein the target at least partially single-stranded viral genome is from a virus selected from the group consisting of Severe Acute Respiratory Syndrome Coronavirus 2 (SARS-CoV-2), Severe Acute Respiratory Syndrome Coronavirus Urbani (SARS-CoV-Urbani), Middle East Respiratory Syndrome Coronavirus (MERS-CoV), Yellow Fever virus, Dengue virus 1, Dengue virus 2, Dengue virus 3, Dengue virus 4, Japanese Encephalitis virus, West Nile virus, Zika virus, Hepatitis C virus, Chikungunya virus, Ross River virus, Sindbis virus, Rubella virus, Norwalk virus, Coxsackievirus, Enterovirus, Rhinovirus, Poliovirus, Ebola virus, Lassa virus, Marburg virus, Hendravirus, Nipavirus, Newcastle disease virus, Parainfluenza virus, Mumps virus, Measles virus, Rabies virus, Human respiratory syncytial virus, Human parvovirus B19, Foot-and-mouth disease virus, Vesicular stomatitis virus, Human Immunodeficiency virus, Influenzavirus, and Hepatitis A virus.

19. The method of claim 1 further comprising:
quantifying the readout signal to determine the presence and/or concentration of the target at least partially single-stranded viral genome in the sample.

20. The method of claim 19, wherein the presence of the target at least partially single-stranded viral genome in the sample is determined.

21. The method of claim 20, wherein the presence of the target at least partially single-stranded viral genome in the sample is determined by a procedure comprising:
calculating an initial rate of readout signal production;
calculating what time period is needed to achieve peak readout signal; and
calculating peak readout signal amplitude or integrated readout signal from time zero to peak readout signal.

22. The method of claim 21 further comprising:
comparing the readout signal to that of a control reaction that lacks the target at least partially single-stranded viral genome.

23. The method of claim 19, wherein the concentration of the target at least partially single-stranded viral genome in the sample is determined.

24. The method of claim 1 or claim 2, wherein the polymerase extension mixture is substantially free of unmodified deoxyadenosine triphosphate (dATP).

25. The method of claim 24, wherein the polymerase extension mixture comprises 2′-Deoxyadenosine-5-(α-thio)-triphosphate, Sodium salt (dATPαS).

26. The method of claim 1, wherein the polymerase extension mixture is substantially free of modified or unmodified forms of dATP.

27. The method of claim 1, wherein the sample is selected from the group consisting of blood, a blood product, plasma, serum, urine, cerebrospinal fluid, saliva, sputum, mucus/cells from nasopharyngeal, oropharyngeal, or nasal-mid-turbinate swabs, tissue, and a synthetic material.

28. The method of claim 1, wherein the method is carried out in solution.

29. The method of claim 1, wherein multiple oligonucleotide molecules are provided for hybridizing to different complementary portions of the target at least partially single-stranded viral genome adjacent to different single-stranded template regions of the target at least partially single-stranded viral genome.

30. The method of claim 29, wherein the different single-stranded template regions are uracil/thymine-free regions.

31. The method of claim 1, wherein the target at least partially single-stranded viral genome is single-stranded.

32. The method of claim 1, wherein the target at least partially single-stranded viral genome is partially single-stranded.

33. The method of claim 1, wherein said providing a sample containing a target at least partially single-stranded viral genome comprises:
collecting the sample from a human, non-human primate, cat, dog, ferret, pig, goat, sheep, cattle, chicken, turkey, duck, camelid, reptile, tiger, bat, pangolin, or amphibian.

34. The method of claim 1, wherein said providing a sample containing a target at least partially single-stranded viral genome comprises:
collecting the sample ex vivo from a surface, material, or inanimate object.

35. A kit for detecting a target at least partially single-stranded viral genome in a sample, said kit comprising:
a polymerase;
a dNTP mixture;
one or more enzymes for producing adenosine triphosphates from released free phosphates; and
a luciferase for producing a bioluminescent readout signal.

36. The kit of claim 35 further comprising one or more oligonucleotide molecules complementary to a selected portion of a target at least partially single-stranded viral genome adjacent to a single-stranded template region of the target at least partially single-stranded viral genome.

37. The kit of claim 36, wherein the single-stranded template region is a uracil/thymine-free region.

38. The kit of claim 36 or claim 37, wherein the kit comprises multiple oligonucleotide molecules for hybridizing to different complementary portions of the target at least partially single-stranded viral genome adjacent to different single-stranded template regions of the target at least partially single-stranded viral genome.

39. The kit of any one of claims 35-38, wherein the different single-stranded template regions are uracil/thymine-free regions.

40. The kit of any one of claims 35-39 further comprising:
a solid support coupled to said polymerase.

41. The kit of any one of claims 35-40 further comprising:
a solid support coupled to said luciferase.

42. The kit of any one of claims 35-41 further comprising:
a solid support coupled to said one or more enzyme(s) for producing adenosine triphosphates from released free phosphates.

43. The kit of claim 35 or claim 26, wherein the polymerase is a reverse transcriptase.

44. The kit of claim 35 or claim 36, wherein the polymerase is a DNA polymerase.

45. The kit of claim 35 or claim 36, wherein the target at least partially single-stranded viral genome is from a virus selected from the group consisting of Severe Acute Respiratory Syndrome Coronavirus 2 (SARS-CoV-2), Severe Acute Respiratory Syndrome Coronavirus Urbani (SARS-CoV-Urbani), Middle East Respiratory Syndrome Coronavirus (MERS-CoV), Yellow Fever virus, Dengue virus 1, Dengue virus 2, Dengue virus 3, Dengue virus 4, Japanese Encephalitis virus, West Nile virus, Zika virus, Hepatitis C virus, Chikungunya virus, Ross River virus, Sindbis virus, Rubella virus, Norwalk virus, Coxsackievirus, Enterovirus, Rhinovirus, Poliovirus, Ebola virus, Lassa virus, Marburg virus, Hendravirus, Nipavirus, Newcastle disease virus, Parainfluenza virus, Mumps virus, Measles virus, Rabies virus, Human respiratory syncytial virus, Human parvovirus B19, Foot-and-mouth disease virus, Vesicular stomatitis virus, Human Immunodeficiency virus, Influenzavirus, and Hepatitis A virus.

46. The kit of any one of claims 35-45, further comprising a microfluidic device.

47. A reaction mixture comprising:
a target at least partially single-stranded viral genome;
one or more oligonucleotide molecules, each oligonucleotide molecule complementary to a selected portion of the target at least partially single-stranded viral genome adjacent to a single-stranded template region of the target at least partially single-stranded viral genome;
a hybridization product comprising a double-stranded nucleic acid start portion in the viral genome at a location corresponding to the selected portion and the single-stranded template region is adjacent to the double-stranded nucleic acid start portion;
a polymerase; and
a dNTP mixture that is substantially free of an unmodified form of dATP.

48. The reaction mixture of claim 47, wherein the single-stranded template region is a uracil/thymine-free region.

49. The reaction mixture of claim 47, wherein the dNTP mixture comprises dATPαS.

50. The reaction mixture of claim 47, wherein the dNTP mixture is substantially free of modified or unmodified forms of dATP.

51. The reaction mixture of claim 47 further comprising:
free phosphates.

52. The reaction mixture of claim 47 further comprising:
glyceraldehyde 3-phosphate dehydrogenase and phosphoglycerate kinase.

53. The reaction mixture of claim 47 further comprising:
a luciferase.

54. A method of treating a human, said method comprising:
obtaining a sample from a human;
determining a presence or an absence of at least a portion of a single-stranded viral genome in the sample by one or more of the methods of claims 1-34; and
treating the human based on the presence or absence of at least a portion of the single-stranded genome in the sample.

55. A method of treating a non-human subject, said method comprising:
obtaining a sample from a non-human subject;
determining a presence or an absence of at least a portion of a single-stranded viral genome in the sample by one or more of the methods of claims 1-34; and
treating the non-human subject based on the presence or absence of at least a portion of the single-stranded genome in the sample.
